(19) United States
(12) Patent Application Publication
(10) Pub. No.: US 2026/0103440 A1
(43) Pub. Date: Apr. 16, 2026

(54) ONIUM SALT, CHEMICALLY AMPLIFIED RESIST COMPOSITION AND PATTERN FORMING PROCESS

(71) Applicant: Shin-Etsu Chemical Co., Ltd., Tokyo (JP)

(72) Inventors: Masahiro Fukushima, Joetsu-shi (JP); Jun Hatakeyama, Joetsu-shi (JP); Yutaro Otomo, Joetsu-shi (JP)

(73) Assignee: Shin-Etsu Chemical Co., Ltd., Tokyo (JP)

(21) Appl. No.: 19/247,129

(22) Filed: Jun. 24, 2025

(30) Foreign Application Priority Data
Jul. 1, 2024 (JP) 2024-106248

(51) Int. Cl. C07C 381/12; G03F 7/00; G03F 7/004

(52) U.S. Cl. CPC C07C 381/12; G03F 7/0045; G03F 7/70033

(57) ABSTRACT

The onium salt has the formula (1). In the formula (1), Xq⁻ is an anion, and the acid (XqH) whose conjugated base is Xq⁻ has a boiling point of lower than 165° C. and a molecular weight of 150 or less. A chemically amplified resist composition comprising the onium salt exhibits a high sensitivity, high resolution, improved lithography properties including EL, LWR, CDU and DOF, and collapse resistance, when processed by lithography using high-energy radiation such as deep UV, EB, or EUV, independent of whether it is of positive or negative tone.

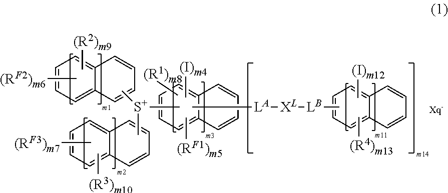

### CROSS-REFERENCE TO RELATED APPLICATION

This non-provisional application claims priority under 35 U.S.C. § 119(a) on Patent Application No. 2024-106248 filed in Japan on Jul. 1, 2024, the entire contents of which are hereby incorporated by reference.

### TECHNICAL FIELD

This invention relates to an onium salt, a chemically amplified resist composition and a pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the most advanced miniaturization technology, mass production of microelectronic devices at the 5-nm node by the lithography using extreme ultraviolet (EUV) having a wavelength of 13.5 nm has been implemented. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is requisite, but the control of acid diffusion is also important (see Non-Patent Document 1). Since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

A triangular tradeoff relationship among sensitivity, resolution, and line width roughness (LWR) of line pattern has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the ArF lithography are typically based on (meth)acrylate polymers having acid labile groups. These acid labile groups undergo deprotection reaction when an acid generator capable of generating a sulfonic acid which is substituted at α-position with fluorine is used, but not when an acid generator capable of generating a sulfonic acid which is not substituted at α-position with fluorine or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Sulfonium salt type quenchers capable of generating carboxylic acid are known. Proposed thus far are sulfonium salts of salicylic acid and β-hydroxycarboxylic acid (Patent Document 4), salicylic acid derivatives (Patent Documents 5 and 6), fluorosalicylic acids (Patent Document 7), hydroxynaphthoeic acid (Patent Document 8), salicylic acids having an iodized aromatic substituent group introduced therein (Patent Document 9), and salicylic acids having a cyclic acetal structure introduced therein (Patent Document 10). Salicylic acid is quite effective for suppressing acid diffusion due to the intramolecular hydrogen bond of carboxy group and hydroxy group.

It is pointed out that the agglomeration of a quencher causes to degrade the dimensional uniformity or CDU of resist patterns. It is thus expected that the CDU of resist patterns after development is improved by preventing the quencher from agglomerating in the resist film, for achieving a uniform distribution of the quencher.

In connection with the demand for further miniaturization, there is a problem that upon development of a positive resist film in an alkaline developer, the resist film is swollen with the developer so that pattern collapse may occur upon small-size pattern formation. To solve the problem associated with miniaturization, the development of an effective material for a new resist composition is important. It is desired to have an onium salt type quencher having a high sensitivity, fully controlled acid diffusion, acceptable solvent solubility, and effective prevention of pattern collapse.

### CITATION LIST

Patent Document 1: JP-A 2006-45311
Patent Document 2: JP-A 2006-178317
Patent Document 3: JP-A 2007-114431
Patent Document 4: WO 2018/159560
Patent Document 5: JP-A 2020-203984
Patent Document 6: JP-A 2020-91404
Patent Document 7: JP-A 2020-91312
Patent Document 8: JP-A 2019-120760
Patent Document 9: JP-A 2022-77505
Patent Document 10: WO 2023/189502
Non-Patent Document 1: SPIE Vol. 6520 65203L-1 (2007)

### SUMMARY OF THE INVENTION

In conjunction with the demand for higher resolution of resist patterns, a prior art resist composition comprising an acid generator of sulfonium salt type and a quencher fails to fully suppress acid diffusion. This raises the problem that lithography properties including contrast, exposure latitude (EL), LWR, CDU, and DOF are degraded. Another problem arising upon formation of small-size patterns is pattern collapse by swell.

An object of the invention is to provide an onium salt and a chemically amplified resist composition comprising the onium salt, the resist composition exhibiting a high sensitivity, high resolution, improved lithography properties including EL, LWR, CDU and DOF, and collapse resistance, when processed by lithography using high-energy radiation such as deep UV, EB, or EUV, independent of whether it is of positive or negative tone. Another object is to provide a pattern forming process using the resist composition.

The inventor has found that an onium salt consisting of a triaryl sulfonium cation containing iodine and fluorine and an anion in which an acid (XqH) as a conjugated base has a boiling point of lower than 165° C. and a molecular weight of 150 or less, and that a chemically amplified resist composition comprising the onium salt as a quencher exhibits a high sensitivity, high contrast, improved lithography properties including EL, LWR, CDU and DOF, and collapse resistance upon formation of small-size patterns.

The invention provides the following onium salts, chemically amplified resist compositions and pattern forming processes.

In one aspect, the invention provides an onium salt having the formula (1).

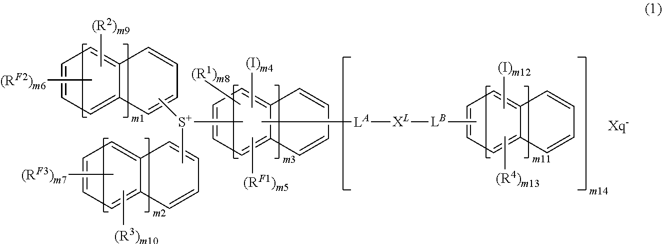

Herein m1 is 0 or 1, m2 is 0 or 1, m3 is 0 or 1, m4 is 0, 1, 2, 3 or 4, m5 is 0, 1, 2, 3 or 4, m6 is 0, 1, 2, 3, 4, 5 or 6, m7 is 0, 1, 2, 3, 4, 5 or 6, m7 is 0, 1, 2, 3, 4, 5 or 6, m8 is 0, 1 or 2, m9 is 0, 1 or 2, m10 is 0, 1 or 2, m11 is 0 or 1, m12 is 0, 1, 2, 3 or 4, m13 is 0, 1 or 2, m14 is 0, 1 or 2, meeting 0≤m6+m9≤4 in case of m1=0 and 0≤m6+m9≤6 in case of m1=1, 0≤m7+m10≤4 in case of m2=0 and 0≤m7+m10≤6 in case of m2=1, 1≤m4+m5+m8+m14≤4 in case of m3=0 and 1≤m4+m5+m8+m14≤6 in case of m3=1, 0≤m12+m13≤4 in case of m11=0 and 0≤m12+m13≤6 in case of m11=1, and m4+m12≥1,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of RF¹ may be identical or different when m5 is 2 or more, a plurality of RF² may be identical or different when m6 is 2 or more, a plurality of RF³ may be identical or different when m7 is 2 or more,
R¹ to R⁴ each are halogen exclusive of iodine and fluorine, nitro group, cyano group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that when m8 is 2, two R¹ may be identical or different and two R¹ may bond together to form a ring with the carbon atoms to which they are attached, when m9 is 2, two R² may be identical or different and two R² may bond together to form a ring with the carbon atoms to which they are attached, when m10 is 2, two R³ may be identical or different and two R³ may bond together to form a ring with the carbon atoms to which they are attached, when m13 is 2, two R⁴ may be identical or different and two R⁴ may bond together to form a ring with the carbon atoms to which they are attached,
the aromatic rings directly bonded to S⁺ in the sulfonium cation may bond together to form a ring with S⁺,
L^{A} and L^{B} are each independently a single bond, ether bond, ester bond, amide bond, sulfonate ester bond, sulfonamide bond, carbonate bond or carbamate bond,
X^{L} is a single bond or a C₁-C₄₀ hydrocarbylene group which may contain a heteroatom, and
Xq⁻ is an anion, with the proviso that an acid (XqH) whose conjugated base is Xq⁻ has a boiling point of lower than 165° C. and a molecular weight of 150 or less.

In a preferred embodiment, the conjugate acid XqH of the anion Xq⁻ is formic acid, acetic acid, propionic acid, butyric acid, trifluoroacetic acid, 3,3,3-trifluoropropionic acid, pivalic acid or nitric acid.

In a preferred embodiment, the onium salt has the formula (1A).

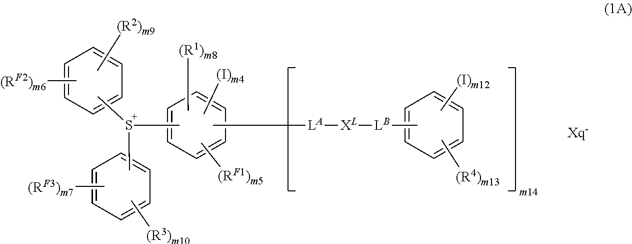

Herein m4 to m10, m12 to m14, R^{F1} to R^{F3}, R¹ to R⁴, L^{A}, L^{B}, X^{L} and Xq⁻ are as defined above.

In a preferred embodiment, the onium salt has the formula (1B).

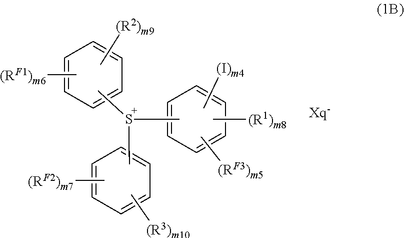

Herein, m4 to m10, R^{F1} to R^{F3}, R¹ to R³ and Xq⁻ are as defined above.

In another aspect, the invention provides a quencher in the form of the onium salt defined herein.

In a further aspect, the invention provides a chemically amplified resist composition comprising the quencher defined herein.

Typically, the chemically amplified resist composition further comprises a base polymer comprising repeat units of at least one type selected from repeat units having the formulae (a1) and (a2).

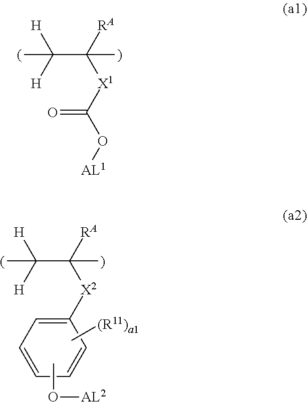

Herein R^{A} is each independently hydrogen, fluorine, methyl or trifluoromethyl,
X¹ is a single bond, phenylene group, naphthylene group, *—C(═O)—O—X¹¹— or *—C(═O)—NH—X¹¹—, the phenylene or naphthylene group may be substituted with a hydroxy moiety, nitro group, cyano group, optionally fluorinated C₁-C₁₀ saturated hydrocarbyl group, optionally fluorinated C₁-C₁₀ saturated hydrocarbyloxy group or halogen, X¹¹ is a C₁-C₁₀ saturated hydrocarbylene group, phenylene group or naphthylene group, the saturated hydrocarbylene group may contain a hydroxy moiety, ether bond, ester bond or lactone ring,
X² is a single bond, *—C(═O)—O— or *—C(═O)—NH—,
* designates a point of attachment to the carbon in the backbone,
R¹¹ is halogen, cyano, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group, a C₂-C₂₀ hydrocarbyloxycarbonyl group which may contain a heteroatom, a plurality of R¹¹ may be identical or different when a1 is 2, 3 or 4,
AL¹ and AL² are each independently an acid labile group, and
a1 is 0, 1, 2, 3 or 4.

In a preferred embodiment, the base polymer further comprises repeat units having the formula (a3).

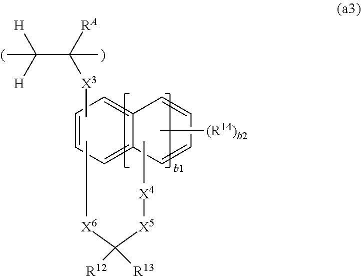

Herein b1 is 0 or 1, and b2 is 0, 1, 2 or 3 in case of b1=0 and 0, 1, 2, 3, 4 or 5 in case of b1=1,
R^{A} is each independently hydrogen, fluorine, methyl or trifluoromethyl,
X³ is a single bond, *—C(═O)—O—, or *—C(═O)—NH—, and * designates a point of attachment to the carbon atom in the backbone,
X⁴ is a single bond, C₁-C₄ aliphatic hydrocarbylene group, carbonyl group, sulfonyl group, or a group obtained by combining the foregoing,
X⁵ and X⁶ are each independently oxygen or sulfur, X⁴ and X⁶ are bonded to vicinal carbon atoms on the aromatic ring,
R¹² and R¹³ are each independently hydrogen, or a C₁-C₂₀ hydrocarbyl group which may contain a heteroatom, R¹² and R¹³ may bond together to form a ring with the carbon atoms to which they are attached,
R¹⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may be identical or different and a plurality of R¹⁴ may bond together to form a ring with the carbon atoms on the aromatic ring to which they are attached when b2 is 2 or more.

In a preferred embodiment, the base polymer further comprises repeat units of at least one type selected from repeat units having the formulae (b1) and (b2).

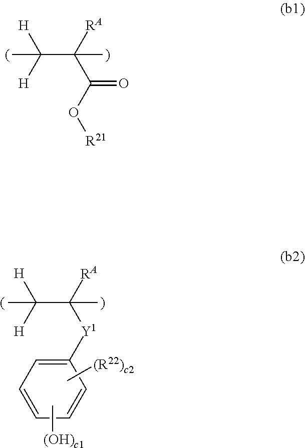

Herein R^{A} is hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R²² is halogen, hydroxy, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3 or 4,
c1 is 1, 2, 3 or 4, c2 is 0, 1, 2, 3 or 4, and c1+c2 is from 1 to 5.

In a preferred embodiment, the base polymer further comprises repeat units of at least one type selected from repeat units having the formulae (c1), (c2), (c3), (c4) and (c5).

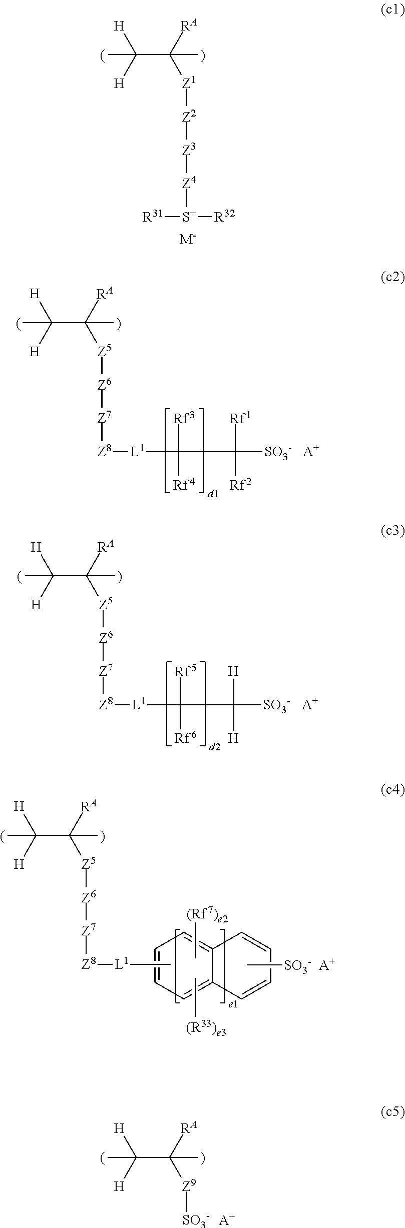

Herein d1 and d2 are each independently 0, 1, 2 or 3, e1 is 0 or 1, e2 is 0, 1, 2, 3 or 4, e3 is 0, 1, 2, 3 or 4, meeting 0≤e2+e3≤4 in case of e1=0 and 0≤e2+e3≤6 in case of e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—NH—Z²¹— or **—O—Z²¹—, Z²¹— is a C₁-C₆ aliphatic hydrocarbylene group, phenylene group or a divalent group obtained by combining the foregoing, which may contain halogen, a carbonyl moiety, ester bond, ether bond or hydroxy moiety,
Z³ is a single bond, ether bond, ester bond, amide bond, sulfonate ester bond, sulfonamide bond, carbonate bond or carbamate bond,
Z⁴ is a C₁-C₆ aliphatic hydrocarbylene group, phenylene group or a divalent group obtained by combining the foregoing, which may contain halogen, a carbonyl moiety, ester bond, ether bond or hydroxy moiety,
Z⁵ is each independently a single bond, optionally substituted phenylene group, naphthylene group, or *—C(═O)—O—Z⁵¹, Z⁵¹ is a C₁-C₁₀ aliphatic hydrocarbylene group which may contain halogen, hydroxy moiety, ether bond, ester bond or lactone ring, or phenylene or naphthylene group,
Z⁶ is a single bond, ether bond, ester bond, amide bond, sulfonate ester bond, sulfonamide bond, carbonate bond or carbamate bond,
Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(═O)—O—, ****—C(═O)—NH—Z⁸¹—, or ****—O—Z⁸¹, Z⁸¹ is a C₁-C₂₀ hydrocarbylene group which may contain a heteroatom,
Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹—, or *—O—Z⁹¹—, Z⁹¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl moiety, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, a C₁-C₆ fluorinated alkoxy group, or a C₁-C₆ fluorinated alkylthio group,
R³¹ and R³² are a C₁-C₂₀ hydrocarbyl group which may contain a heteroatom, R³¹ and R³² may bond together to form a ring with the sulfur atom to which they are attached,
R³³ is halogen exclusive of fluorine, or a C₁-C₂₀ hydrocarbyl group which may contain a heteroatom, a plurality of R³³ may be identical or different and a plurality of R³³ may bond together to form a ring with the carbon atoms to which they are attached when e3 is 2, 3 or 4
M⁻ is a non-nucleophilic counter ion, and
A⁺ is an onium cation.

In a preferred embodiment, the chemically amplified resist composition further comprises an organic solvent.

In a preferred embodiment, the chemically amplified resist composition further comprises a photoacid generator.

In a preferred embodiment, the chemically amplified resist composition further comprises a quencher other than the quencher defined herein.

In a preferred embodiment, the chemically amplified resist composition further comprises a surfactant.

In a further aspect, the invention provides a pattern forming process comprising the steps of applying the chemically amplified resist composition defined herein onto a substrate to form a resist film thereon, exposing the resist film to high-energy radiation, and developing the exposed resist film in a developer.

Typically, the high-energy radiation is KrF excimer laser radiation, ArF excimer laser radiation, EB or EUV of wavelength 3 to 15 nm.

### Advantageous Effects of Invention

When processed by lithography, a chemically amplified resist composition comprising the inventive onium salt as a quencher exhibits a high sensitivity, high contrast, improved lithography properties including EL, LWR, CDU and DOF.

### DESCRIPTION OF THE PREFERRED EMBODIMENT

### Onium Salt

One embodiment of the invention is an onium salt having the formula (1).

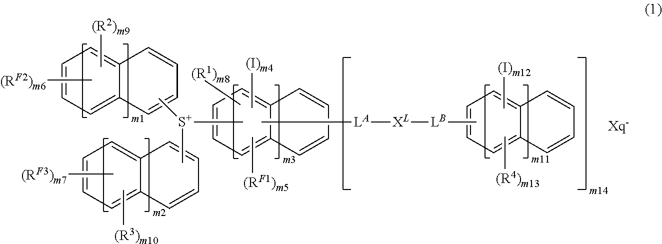

In formula (1), m1 is 0 or 1. The relevant structure is a benzene ring in case of m1=0, and a naphthalene ring in case of m1=1. From the aspect of solvent solubility, the benzene ring corresponding to m1=0 is preferred. The subscript m2 is 0 or 1. The relevant structure is a benzene ring in case of m2=0, and a naphthalene ring in case of m2=1. From the aspect of solvent solubility, the benzene ring corresponding to m2=0 is preferred. The subscript m3 is 0 or 1. The relevant structure is a benzene ring in case of m3=0, and a naphthalene ring in case of m3=1. From the aspect of solvent solubility, the benzene ring corresponding to m3=0 is preferred.

In the formula (1), m4 is 0, 1, 2, 3 or 4. Since a cation structure containing more iodine atoms is more absorptive to EUV, but loses solvent solubility so that it may precipitate in a resist composition, it is preferred that m4 be 0, 1, 2 or 3, more preferably 0, 1 or 2.

In formula (1), m5 is 0, 1, 2, 3 or 4. It is preferred from the aspect of reactant availability that m5 be 0, 1, 2 or 3, more preferably 0, 1 or 2. The subscript m6 is 0, 1, 2, 3, 4, 5 or 6. It is preferred from the aspect of reactant availability that m6 be 0, 1, 2 or 3, more preferably 0, 1 or 2. The subscript m7 is 0, 1, 2, 3, 4, 5 or 6. It is preferred from the aspect of reactant availability that m7 be 0, 1, 2 or 3, more preferably 0, 1 or 2.

In formula (1), m8 is 0, 1 or 2. It is preferred from the aspect of reactant availability that m8 be 0 or 1. The subscript m9 is 0, 1 or 2. It is preferred from the aspect of reactant availability that m9 be 0 or 1. The subscript m10 is 0, 1 or 2. It is preferred from the aspect of reactant availability that m10 be 0 or 1.

In formula (1), m11 is 0 or 1. The relevant structure is a benzene ring in case of m11=0 and a naphthalene ring in case of m11=1. From the aspect of solvent solubility, the benzene ring corresponding to m11=0 is preferred.

In formula (1), m12 is 0, 1, 2, 3 or 4. Since a cation structure containing more iodine atoms is more absorptive to EUV, but loses solvent solubility so that it may precipitate in a resist composition, it is preferred that m12 be 0, 1, 2 or 3, more preferably 0, 1 or 2.

In formula (1), m13 is 0, 1 or 2. It is preferred from the aspect of reactant availability that m13 be 0 or 1. The subscript m14 is 0, 1 or 2. It is preferred from the aspect of synthesis that m14 be 0 or 1.

It is noted that m6+m9 is from 0 to 4 when m1=0, m6+m9 is from 0 to 6 when m1=1; m7+m10 is from 0 to 4 when m2=0, m7+m10 is from 0 to 6 when m2=1; m4+m5+m8+m14 is from 1 to 4 when m3=0, m4+m5+m8+m14 is from 1 to 6 when m3=1; m12+m13 is from 0 to 4 when m11=0, m12+m13 is from 0 to 6 when m11=1; and m4+m12 is 1 or greater.

In formula (1), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl, trifluoromethoxy, and trifluorothiomethoxy are preferred. A plurality of R^{F1} may be identical or different when m6 is 2 or more, a plurality of R^{F2} may be identical or different when m7 is 2 or more, and a plurality of R^{F3} may be identical or different when m5 is 2 or more.

In formula (1), R¹ to R⁴ are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. Examples of the halogen include fluorine, chlorine, bromine and iodine. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. In the hydrocarbyl group, and hydrocarbyl moieties of the hydrocarbyloxy and hydrocarbylthio groups, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

When m8=2, two R¹ may be identical or different and two R¹ may bond together to form a ring with the carbon atoms to which they are attached. When m9=2, two R² may be identical or different and two R² may bond together to form a ring with the carbon atoms to which they are attached. When m10=2, two R³ may be identical or different and two R³ may bond together to form a ring with the carbon atoms to which they are attached. When m13=2, two R⁴ may be identical or different and two R⁴ may bond together to form a ring with the carbon atoms to which they are attached. Specific examples of the ring formed herein include cyclopropane, cyclobutane, cyclopentane, cyclohexane, norbornane and adamantane rings. In the ring, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having formula (1) may bond together to form a ring with S⁺. Exemplary structures of the ring are shown below.

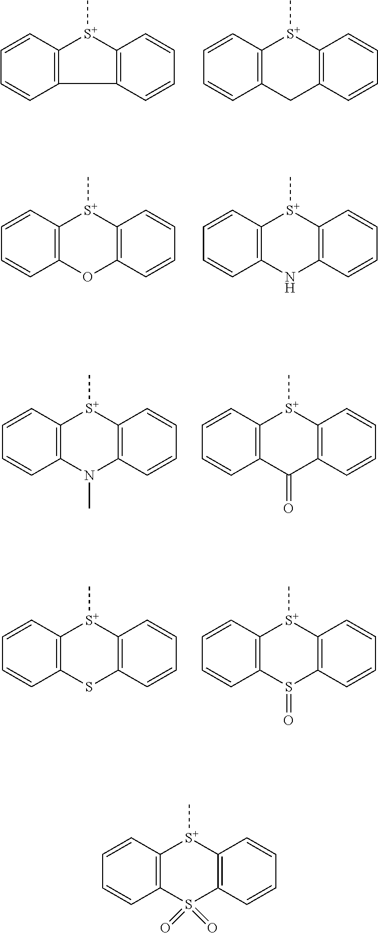

Herein the broken line denotes a point of attachment.

In formula (1), L^{A} and L^{B} are each independently a single bond, ether bond, ester bond, amide bond, sulfonate ester bond, sulfonamide bond, carbonate bond or carbamate bond. L^{A} is preferably a single bond, ether bond, ester bond or sulfonate ester bond, more preferably an ester bond or sulfonate ester bond. L^{B} is preferably a single bond, ether bond or ester bond, more preferably a single bond.

In formula (1), X^{L} is a single bond or a C₁-C₄₀ hydrocarbylene group which may contain a heteroatom. The C₁-C₄₀ hydrocarbylene group may be straight, branched or cyclic. Suitable hydrocarbylene groups include alkanediyl, cyclic saturated hydrocarbylene and arylene groups. Suitable heteroatoms include oxygen, nitrogen and sulfur atoms.

Examples of the optionally heteroatom-containing C₁-C₄₀ hydrocarbylene group X^{L} are shown below, but not limited thereto. Herein * each designates a point of attachment to L^{A} or L^{B}.

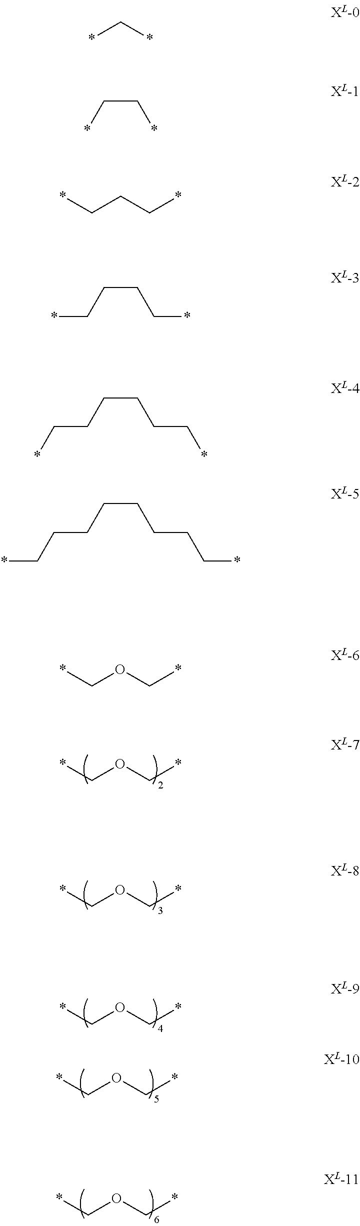

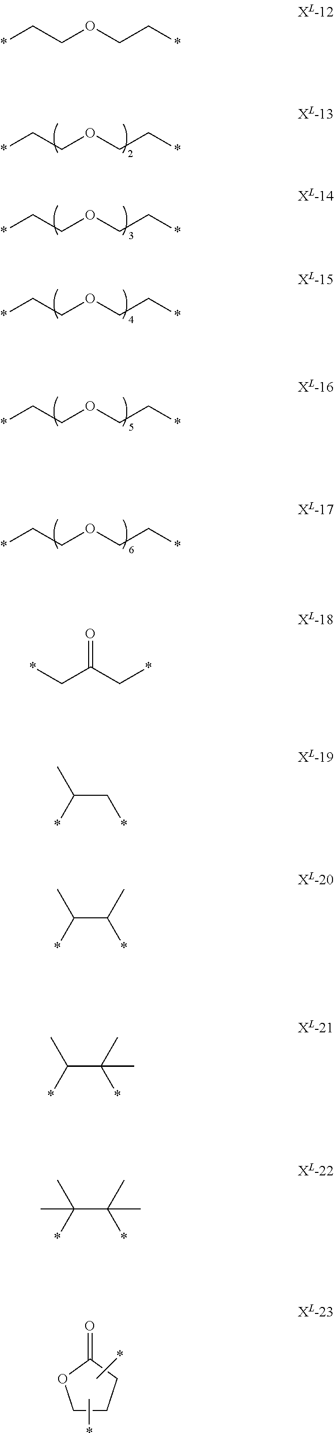

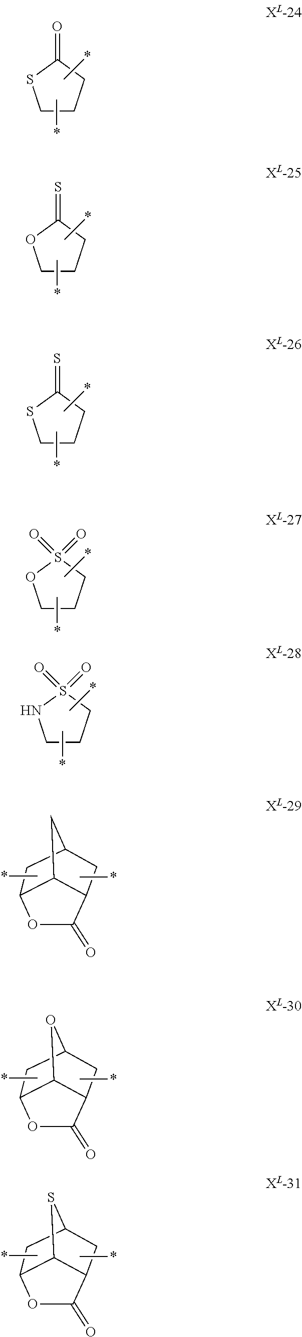

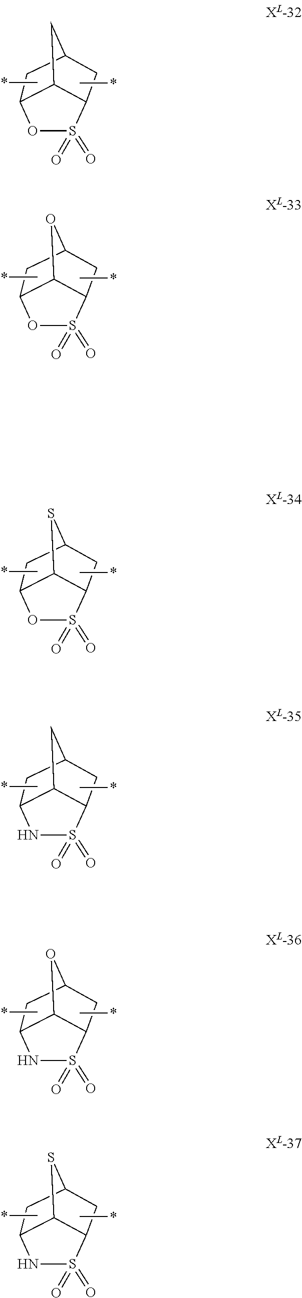

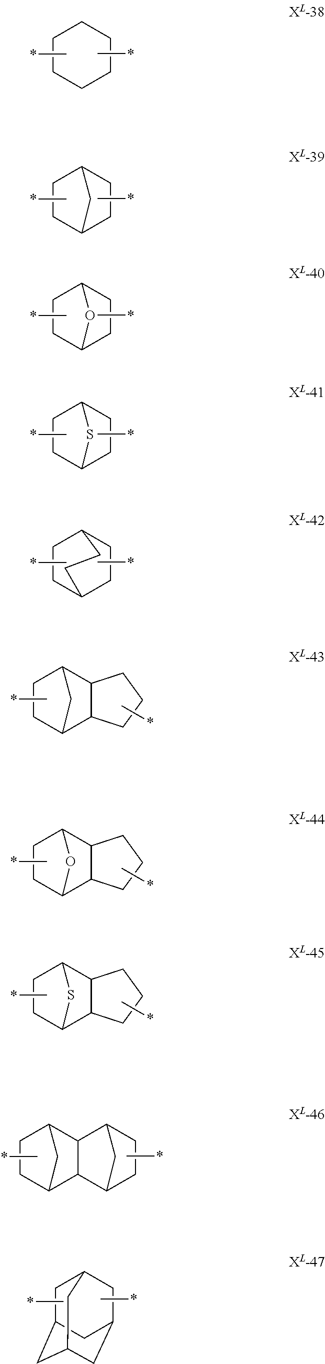

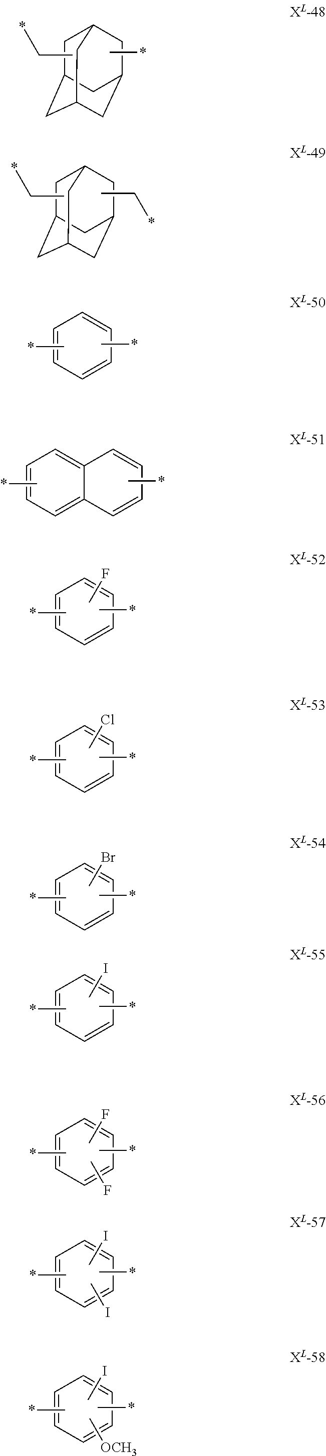

Of these, X^{L}-0 to X^{L}-22, X^{L}-29 to X^{L}-34, and X^{L}-47 to X^{L}-58 are preferred.

Preferably the onium salt having formula (1) has the formula (1A):

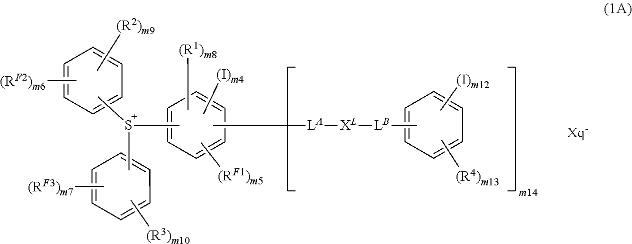

wherein m4 to m10, m12 to m14, R^{F1} to R^{F3}, R¹ to R⁴, L^{A}, L^{B}, X^{L} and Xq⁻ are as defined above.

Preferably the onium salt having formula (1A) has the formula (1B):

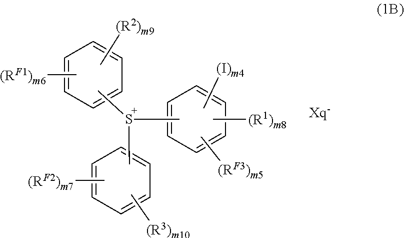

wherein, m4 to m10, R^{F1} to R^{F3}, R¹ to R³ and Xq⁻ are as defined above.

Examples of the cation in the sulfonium salt having formula (1) are shown below, but not limited thereto. In the following formulae, Me is a methyl group.

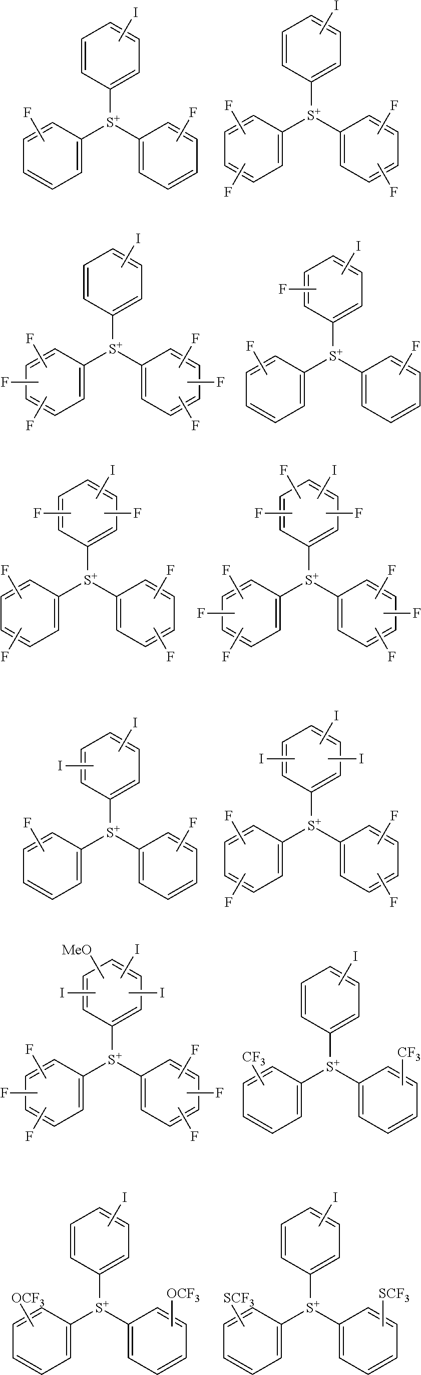

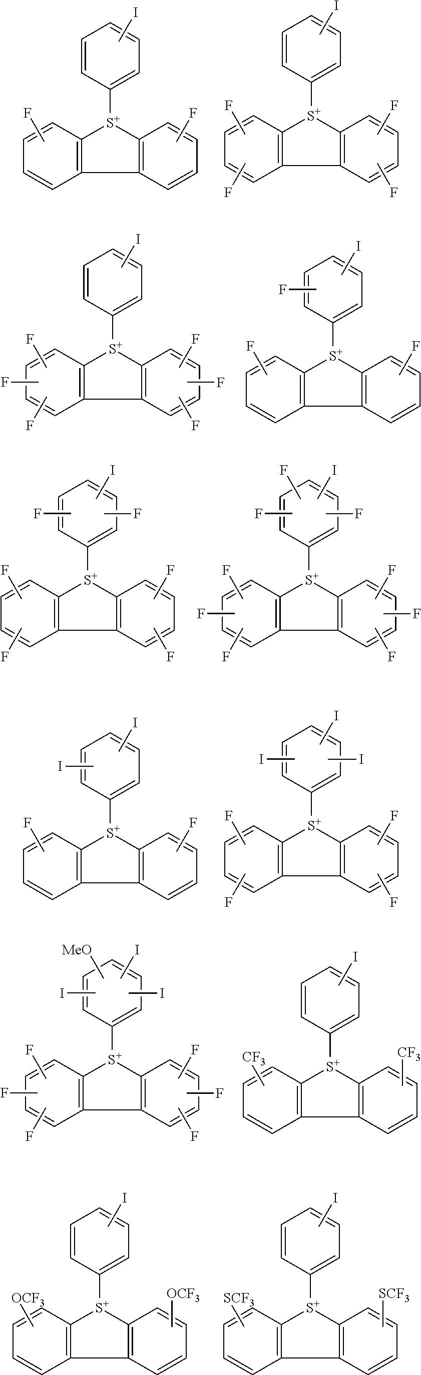

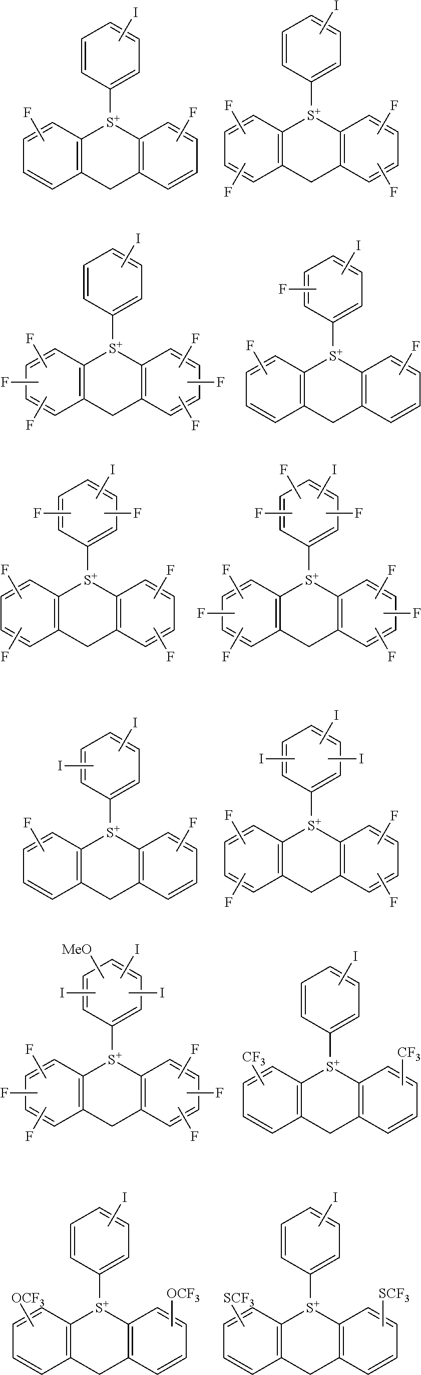

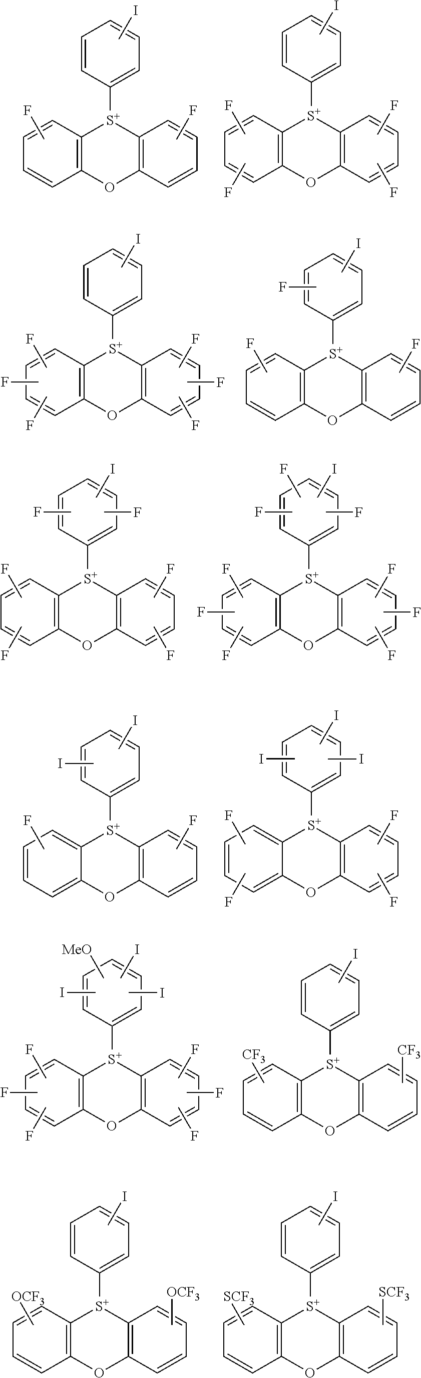

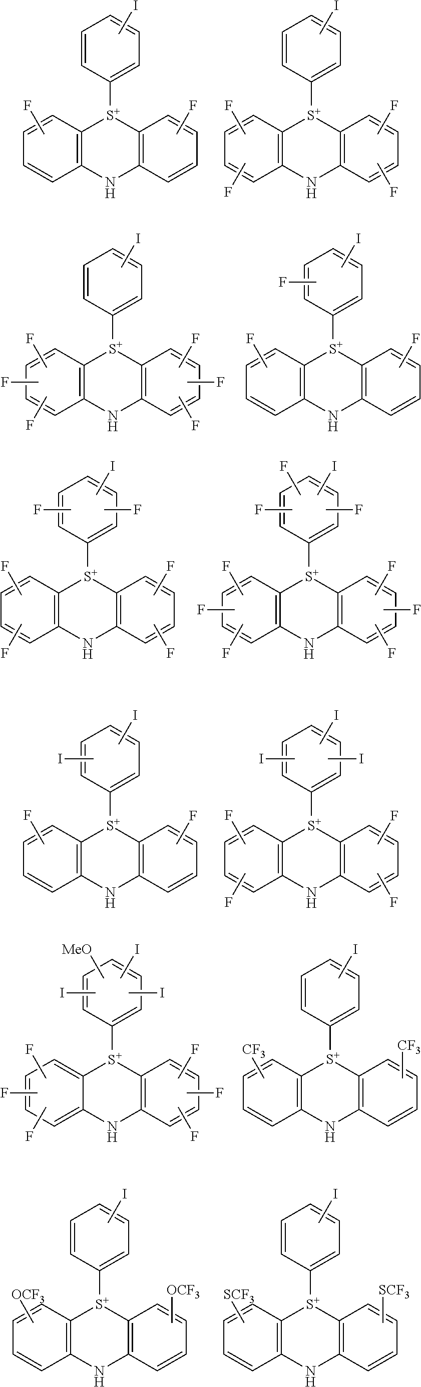

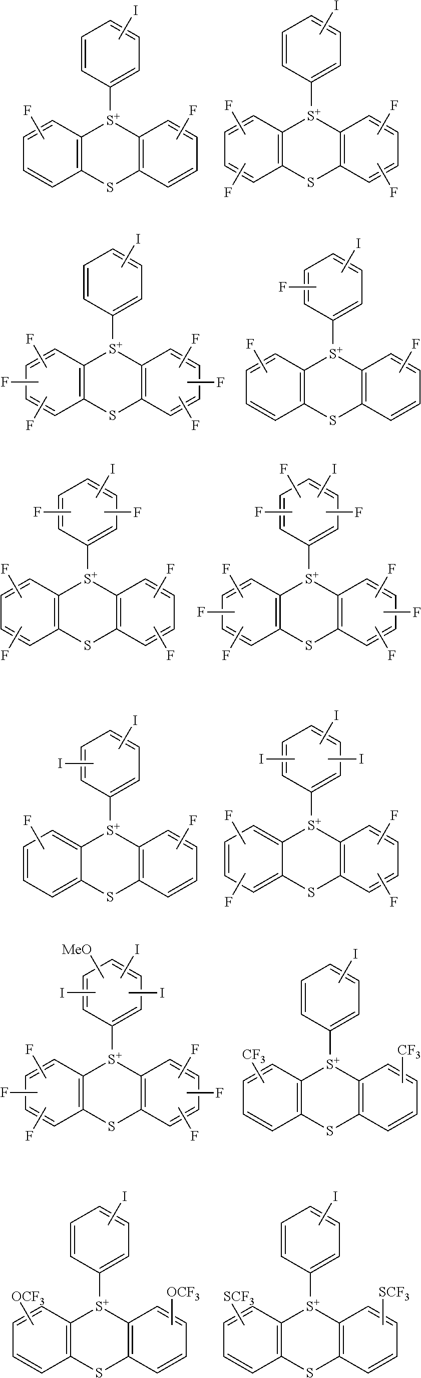

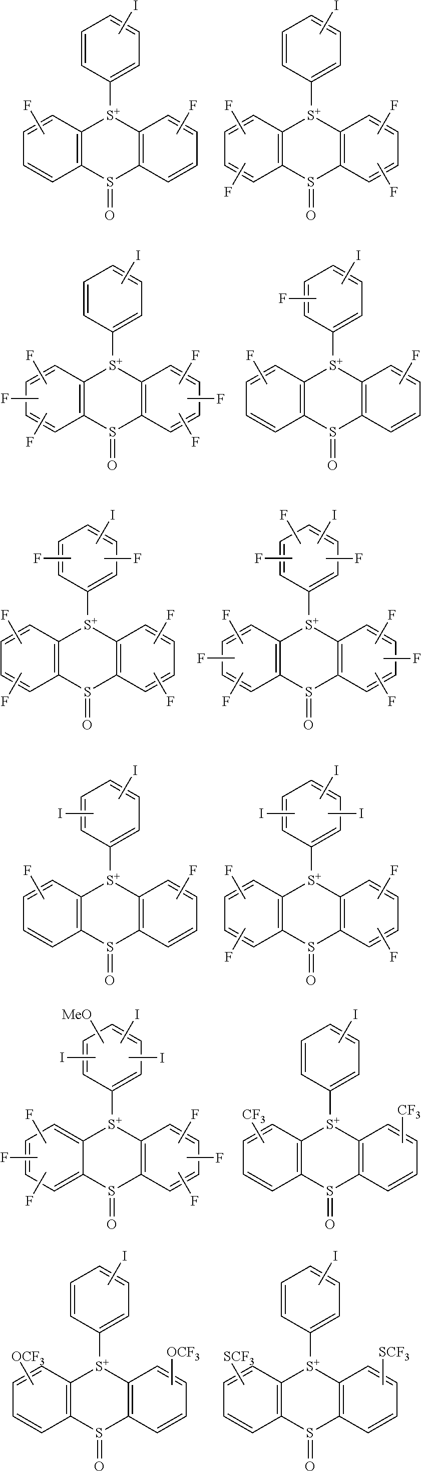

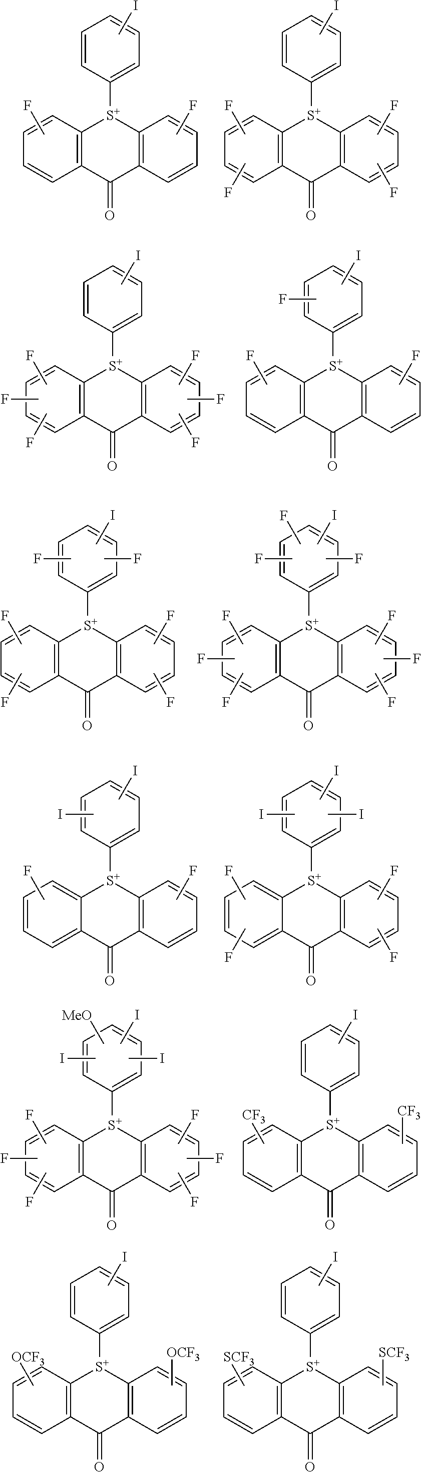

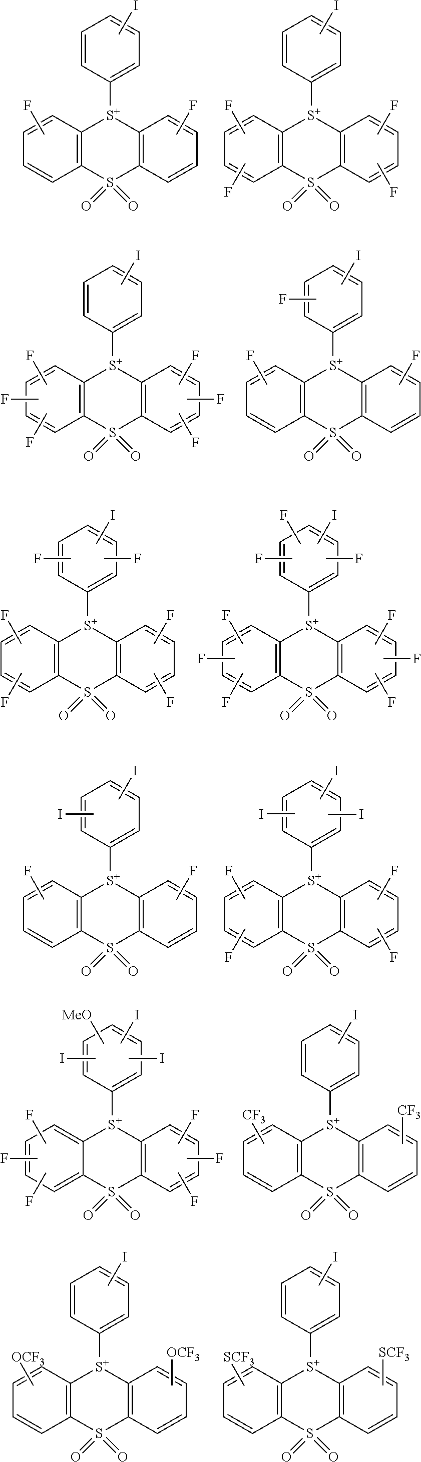

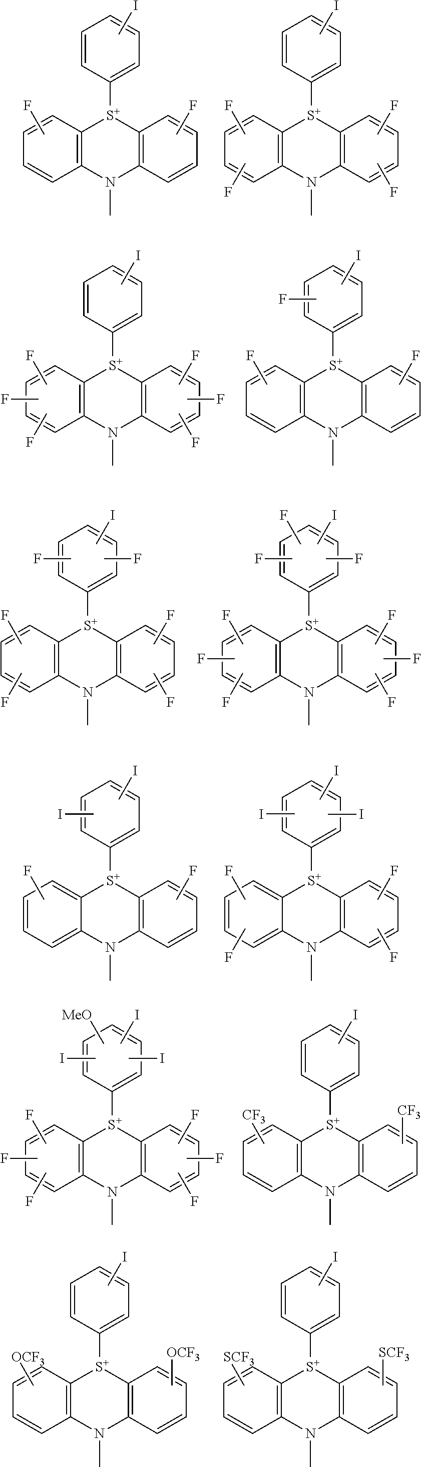

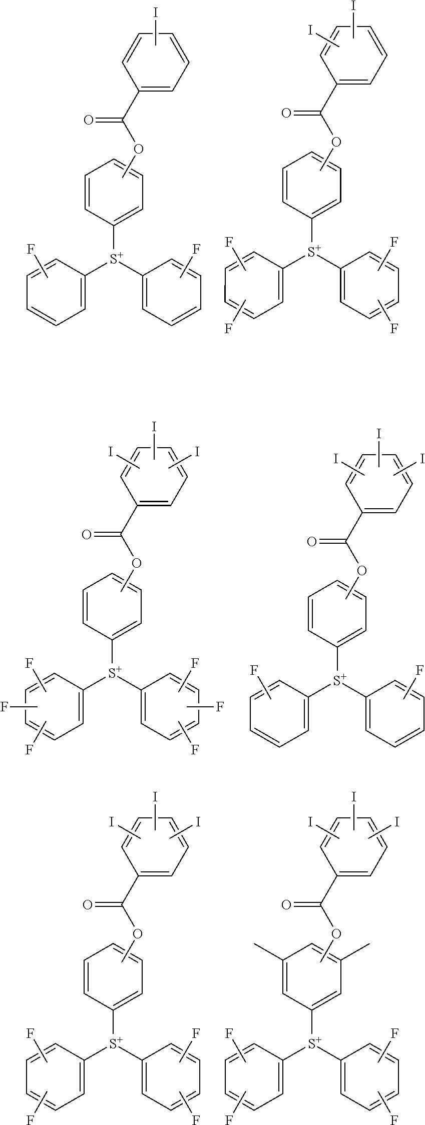

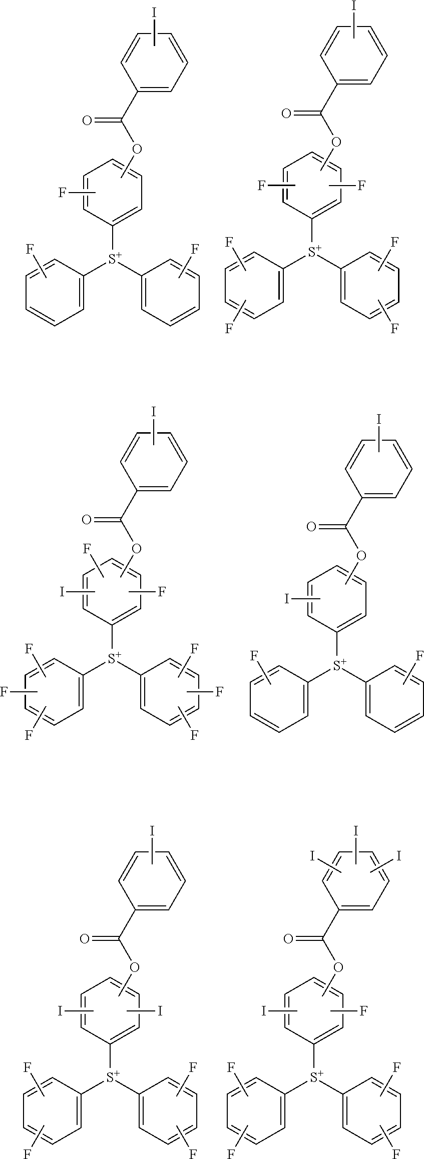

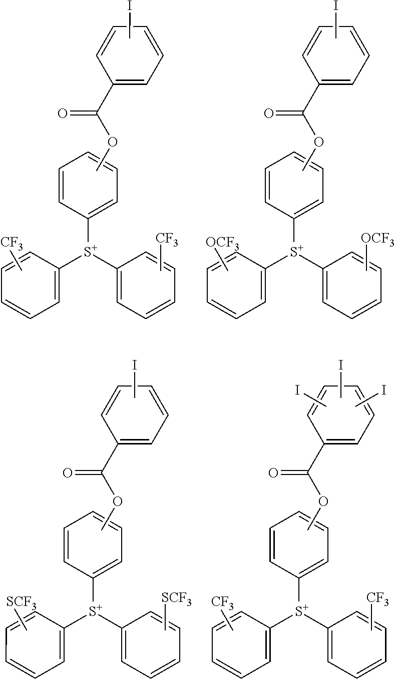

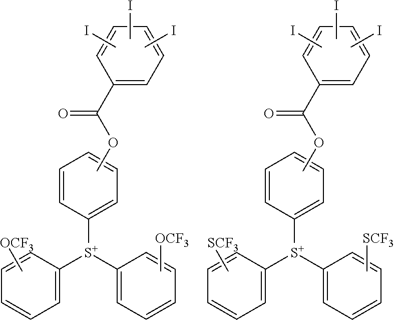

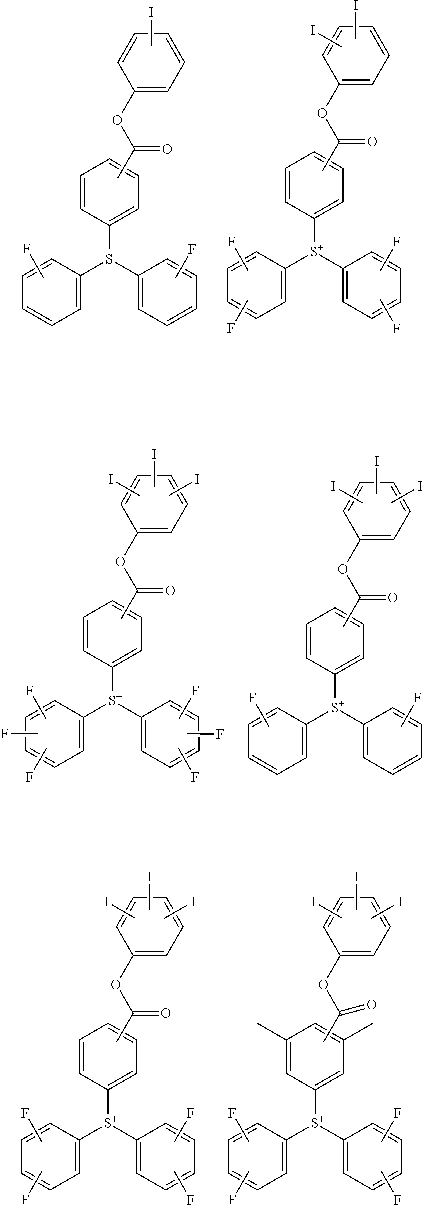

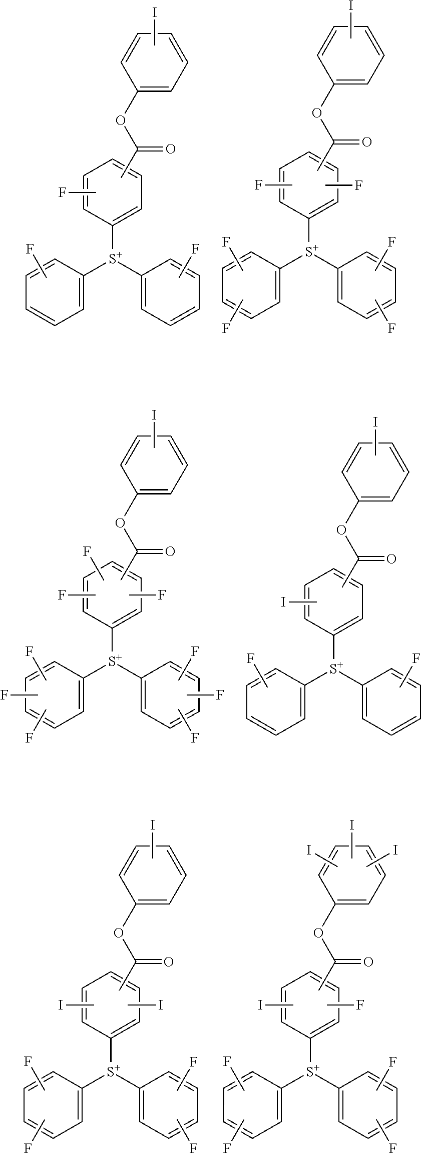

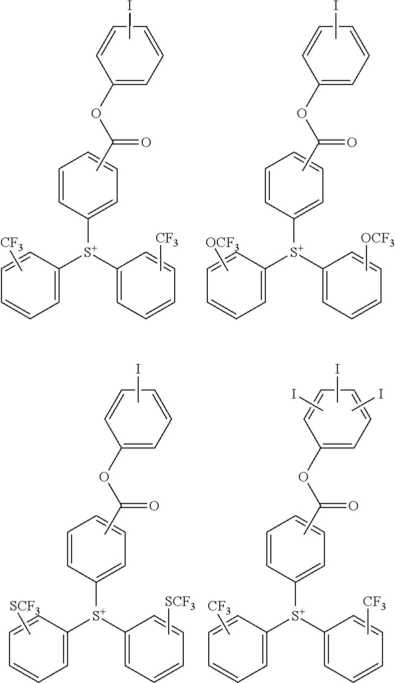

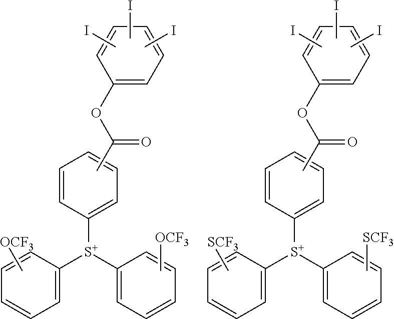

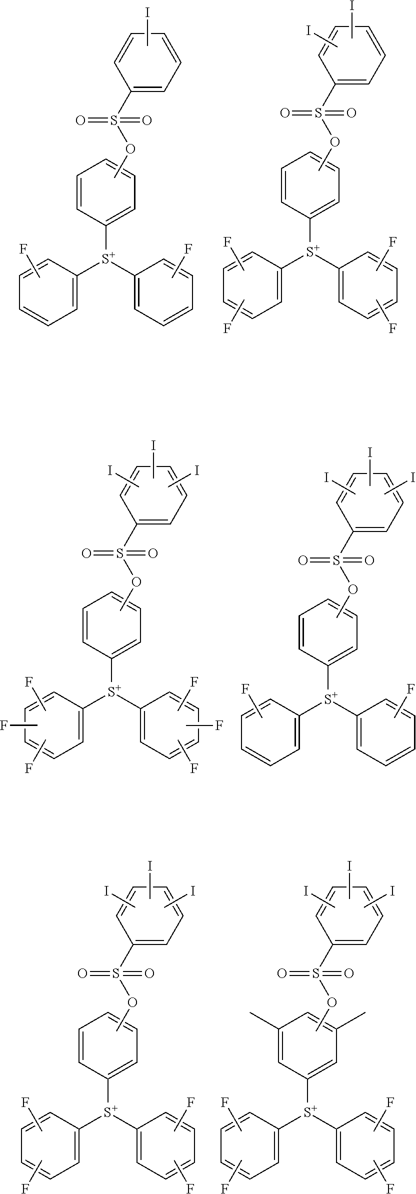

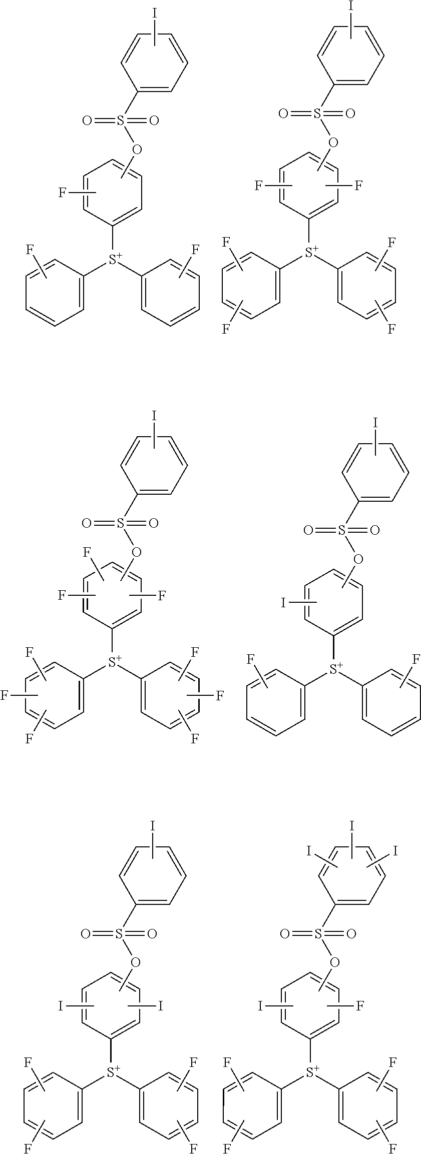

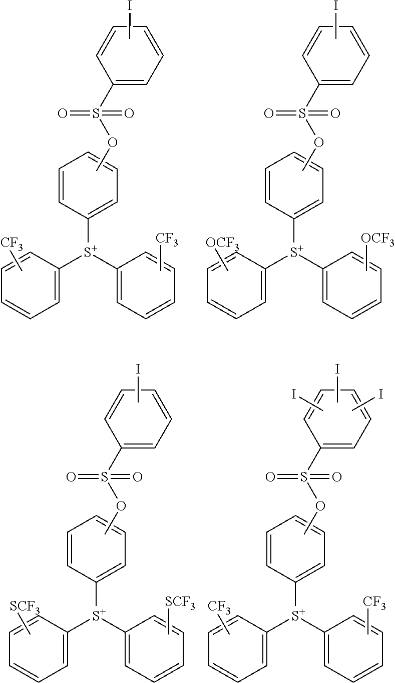

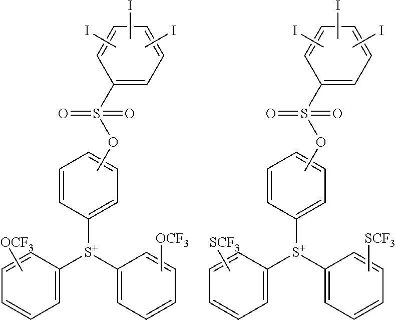

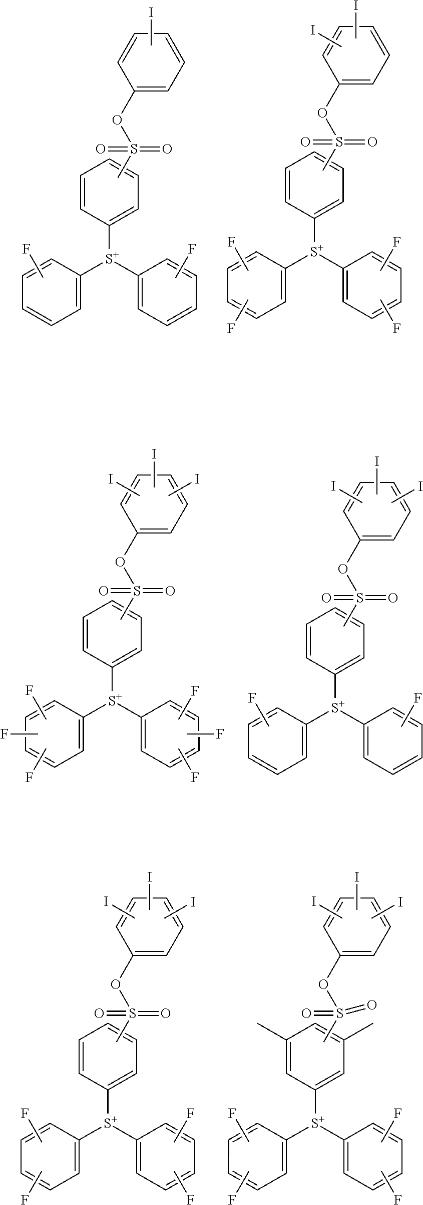

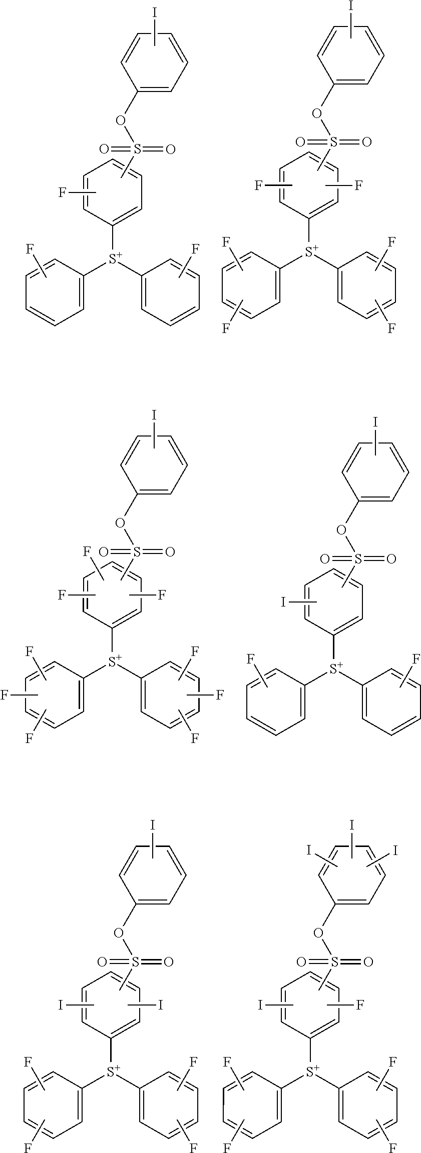

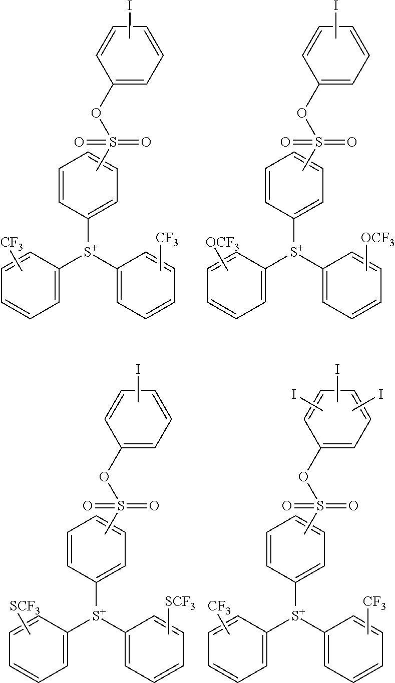

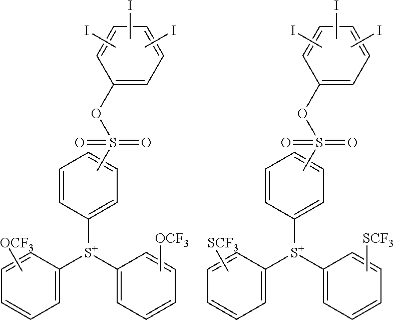

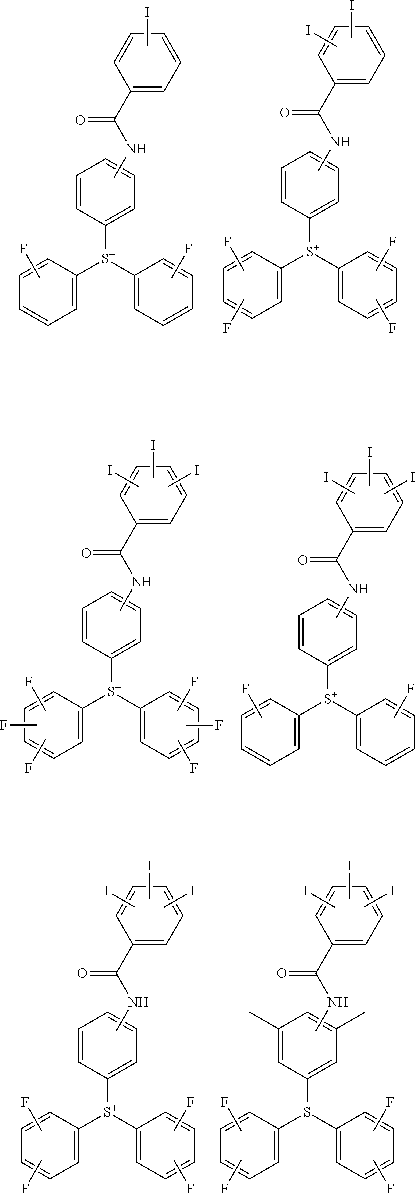

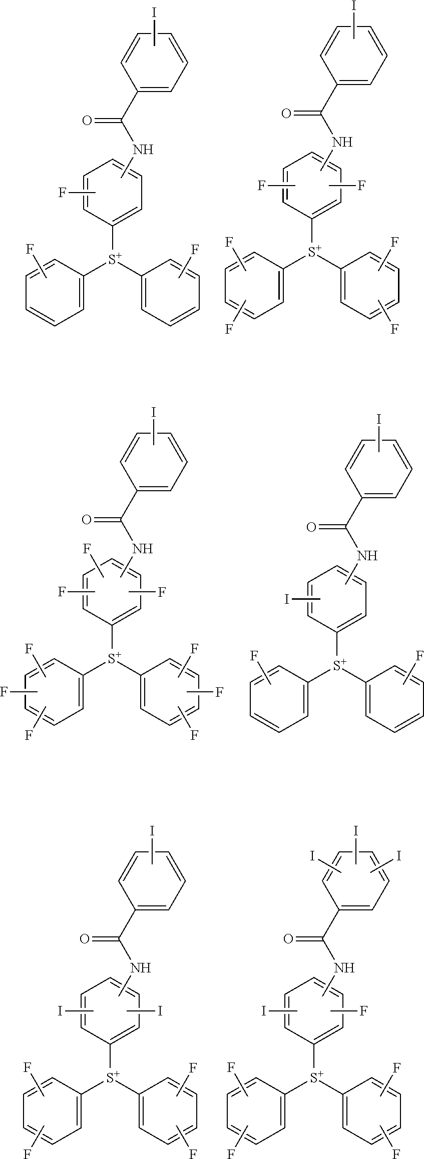

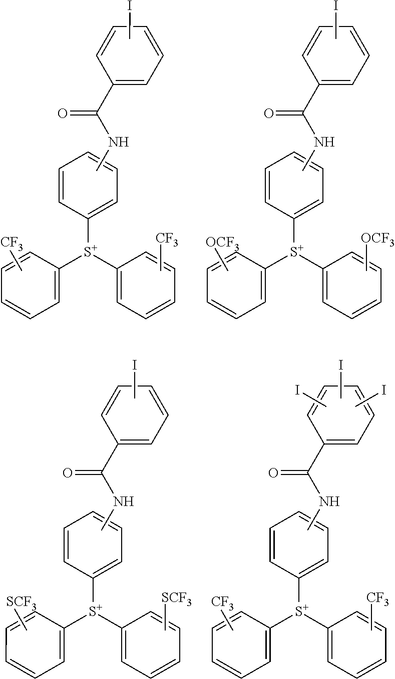

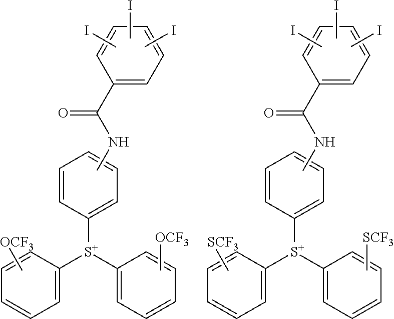

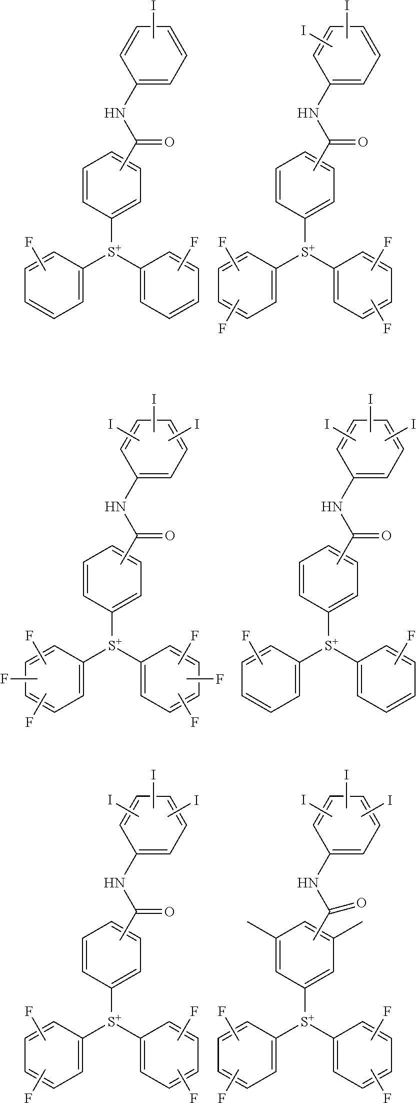

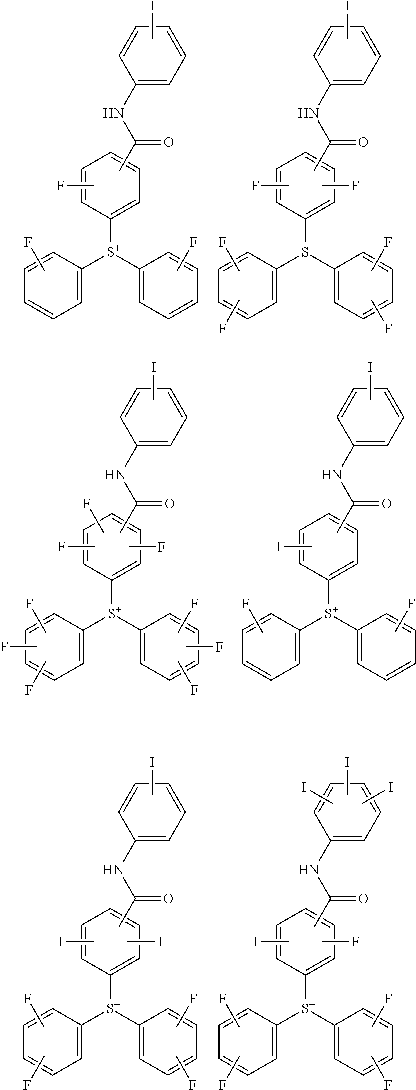

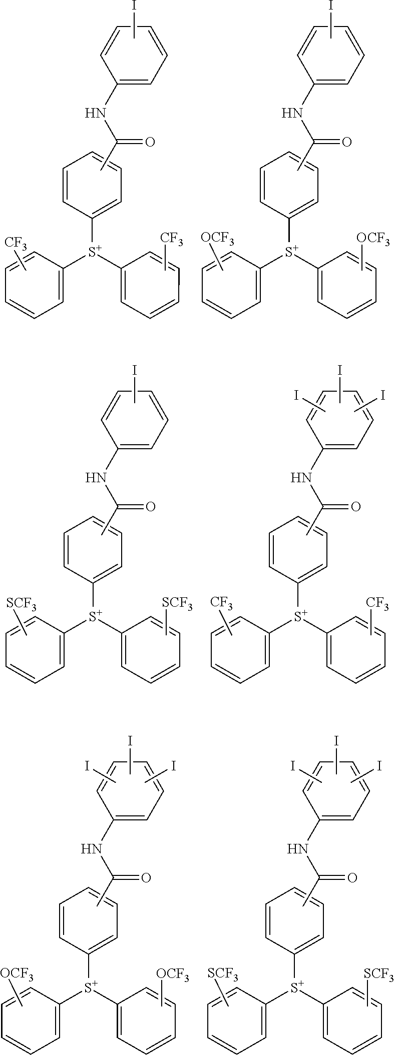

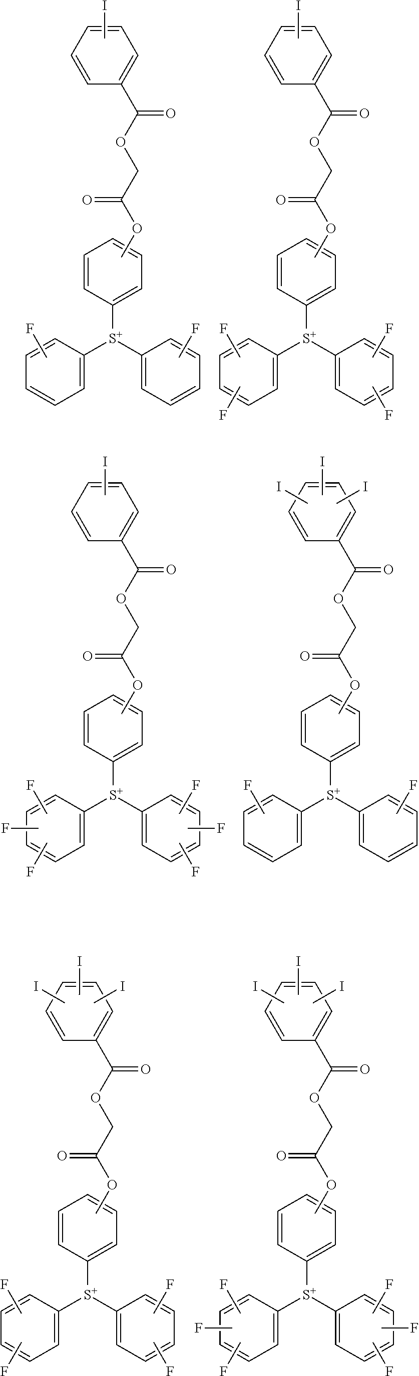

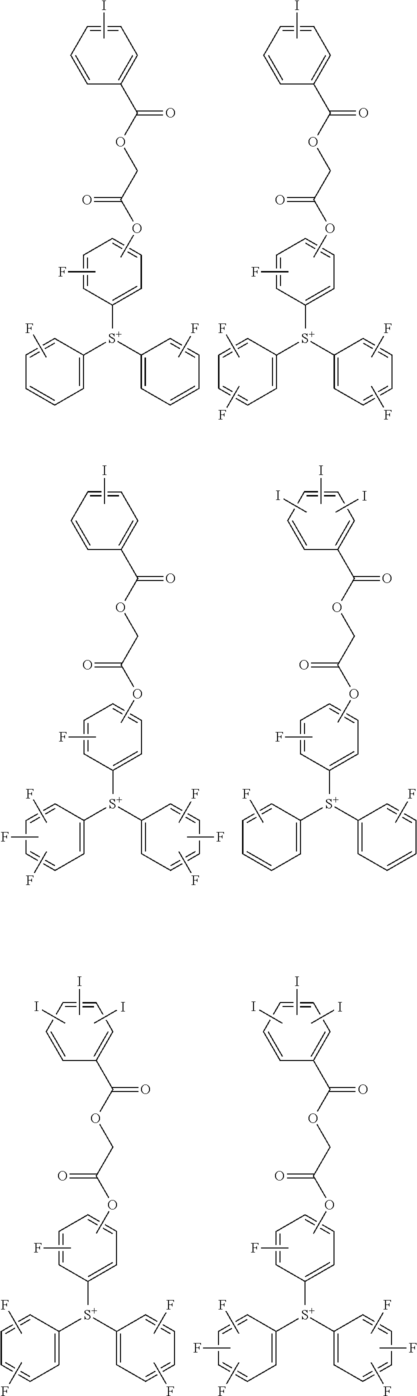

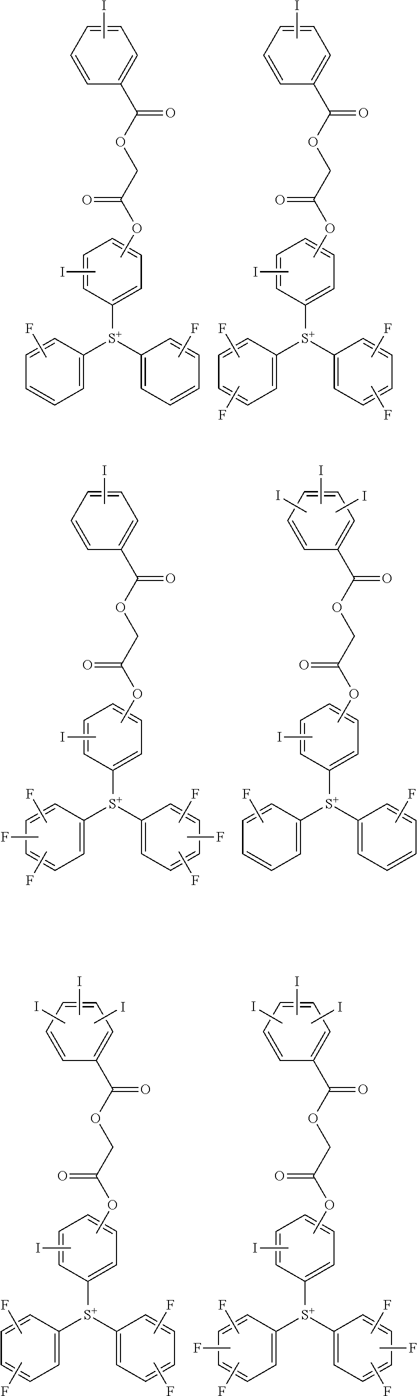

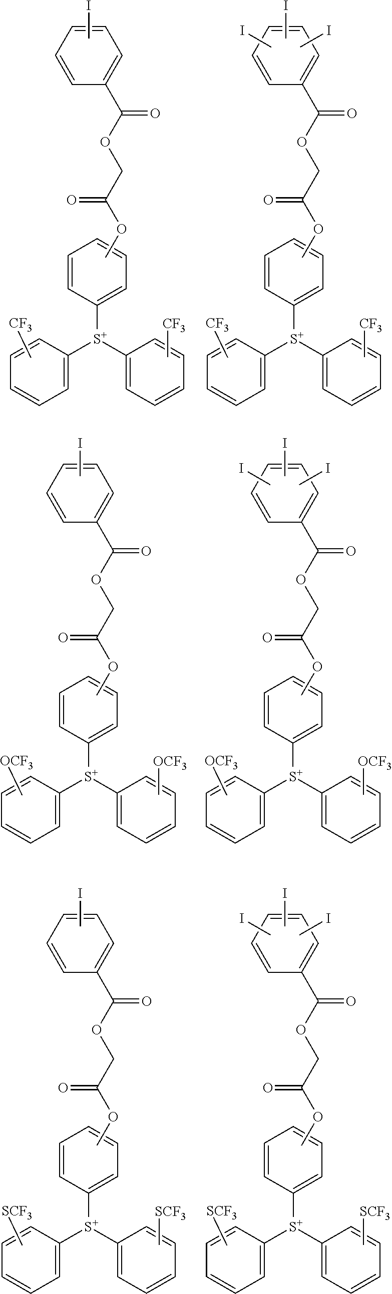

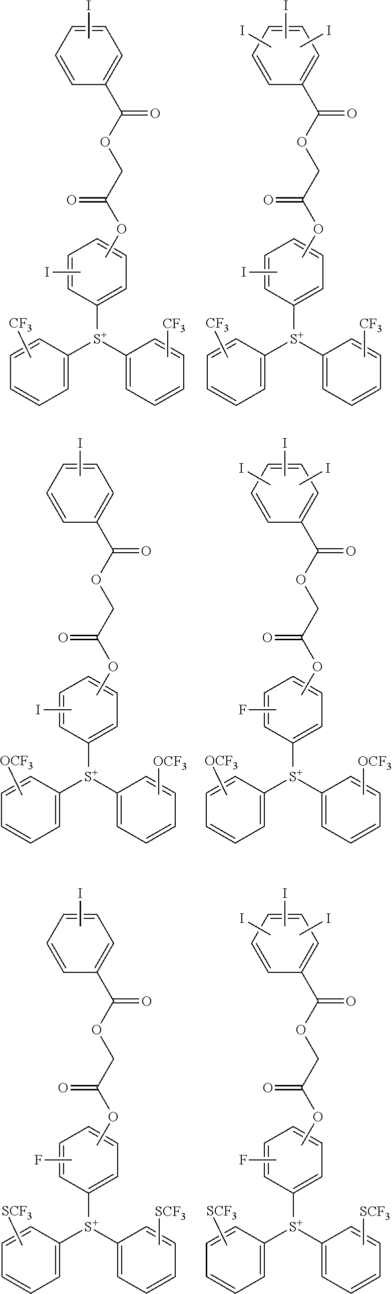

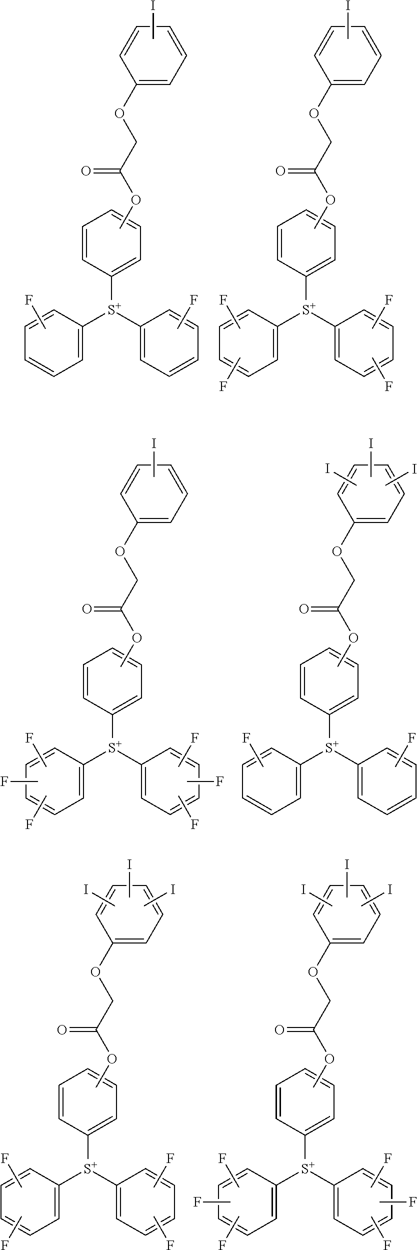

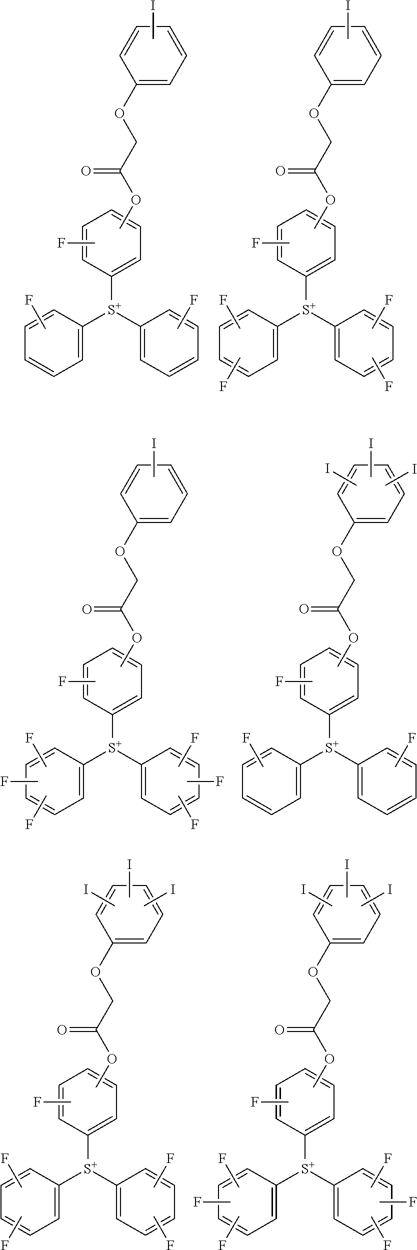

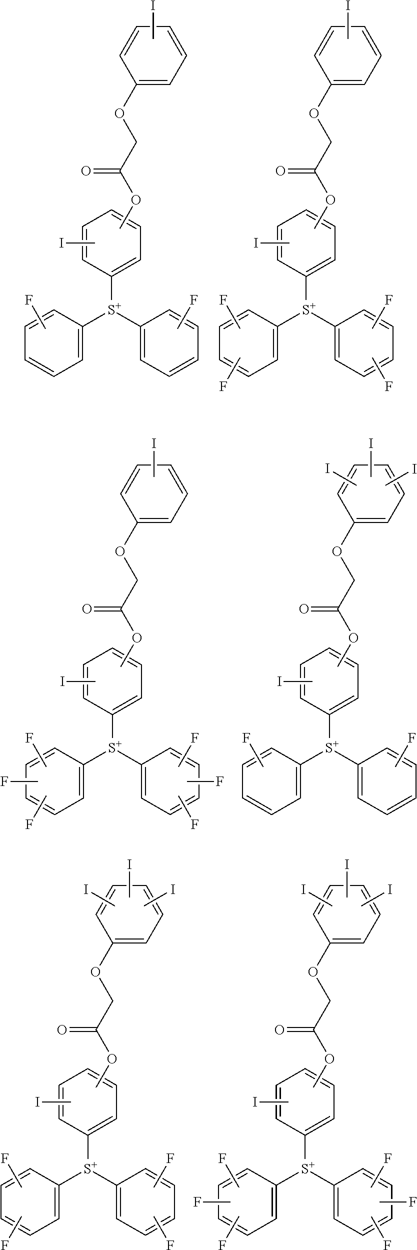

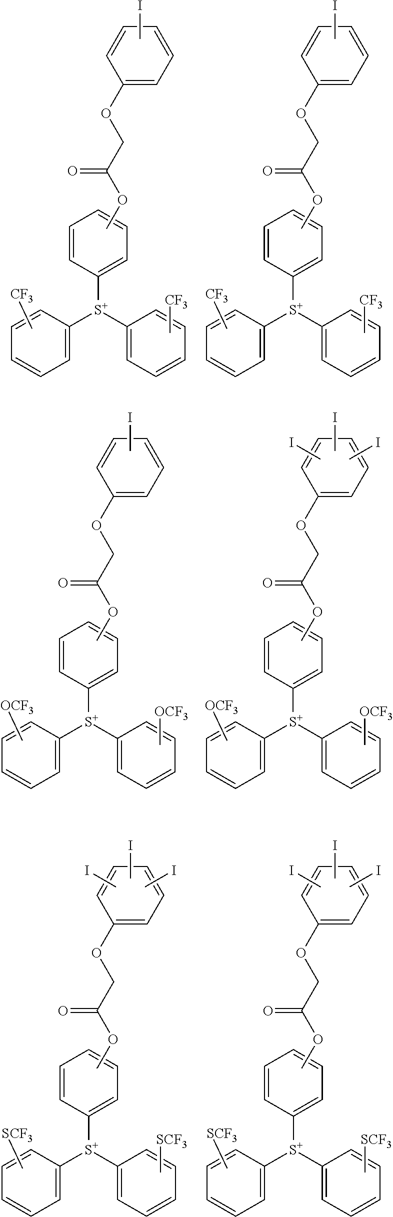

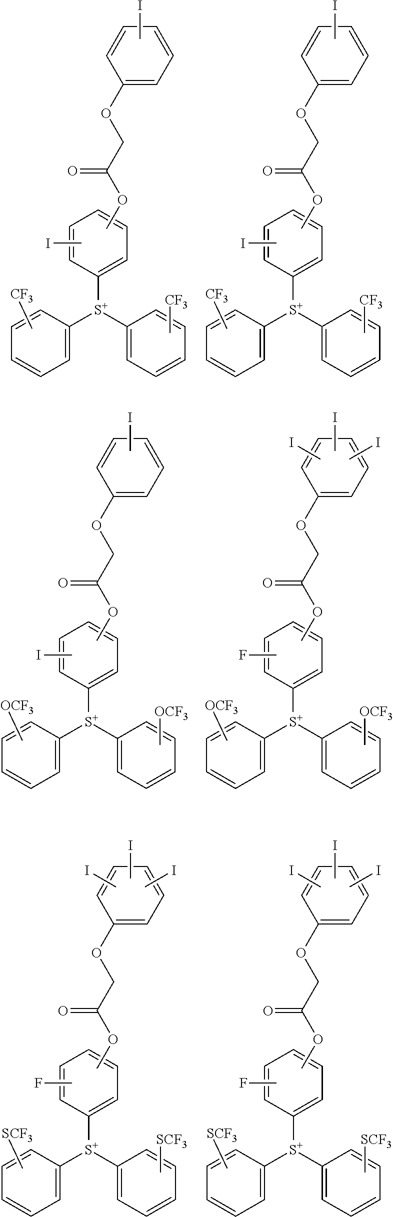

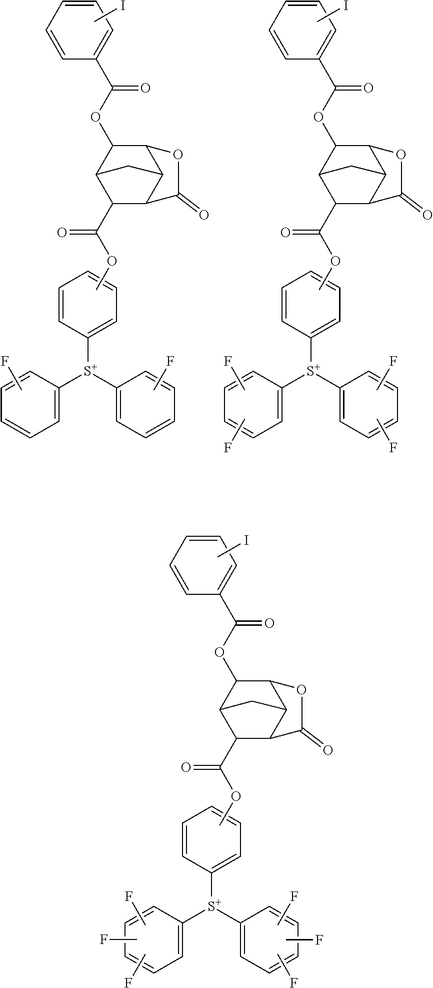

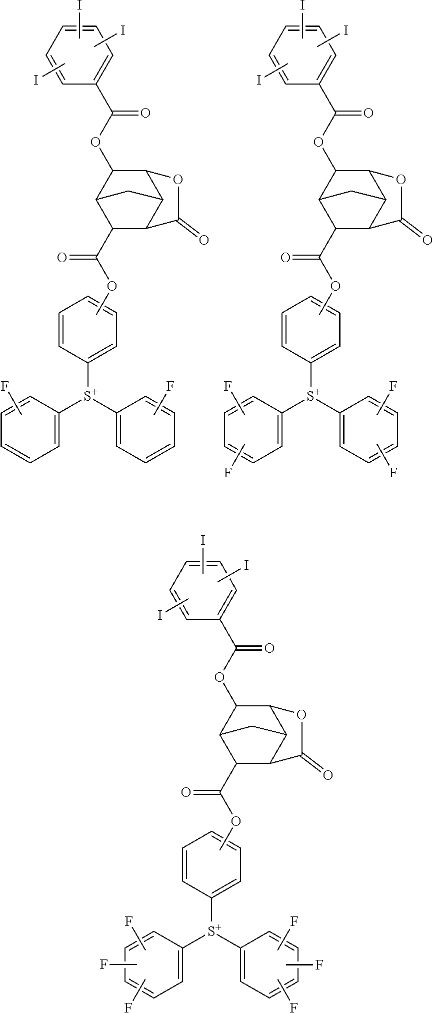

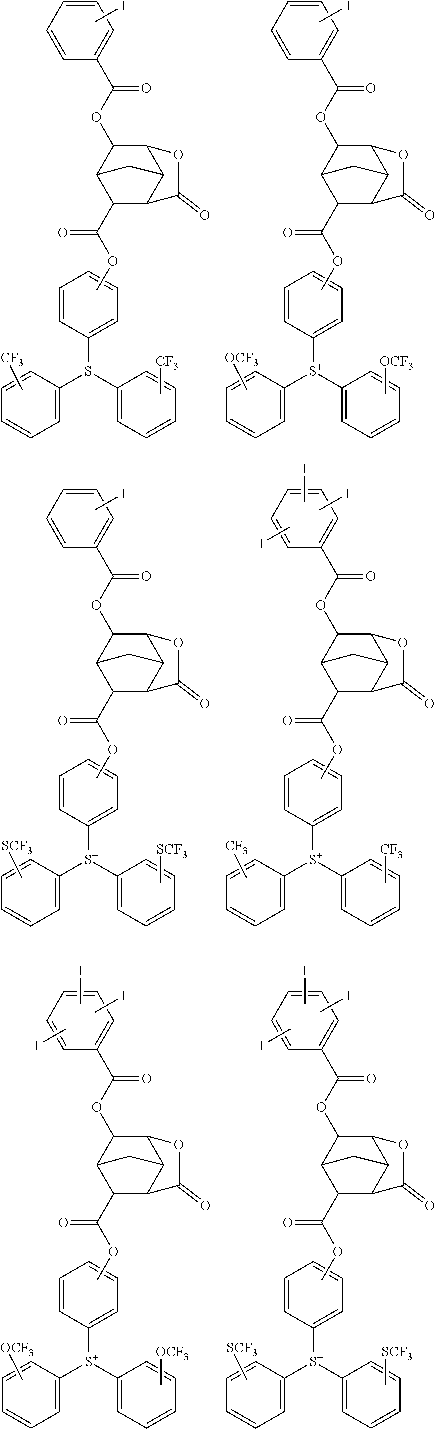

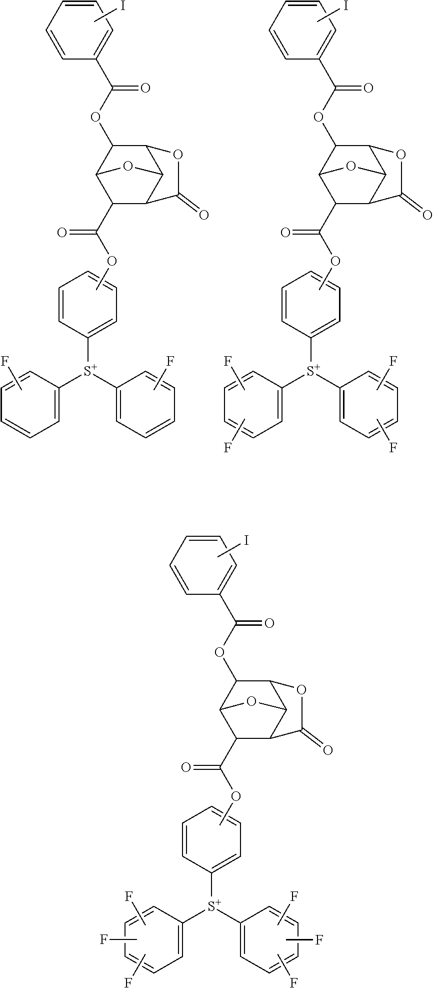

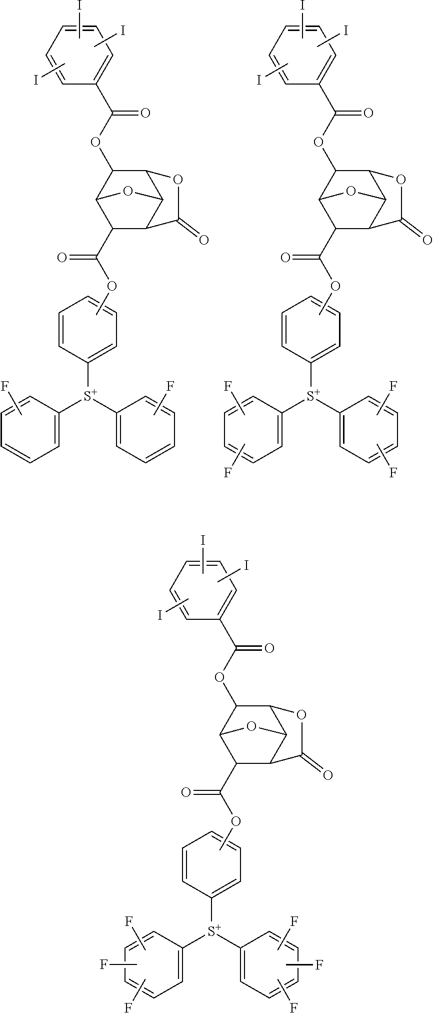

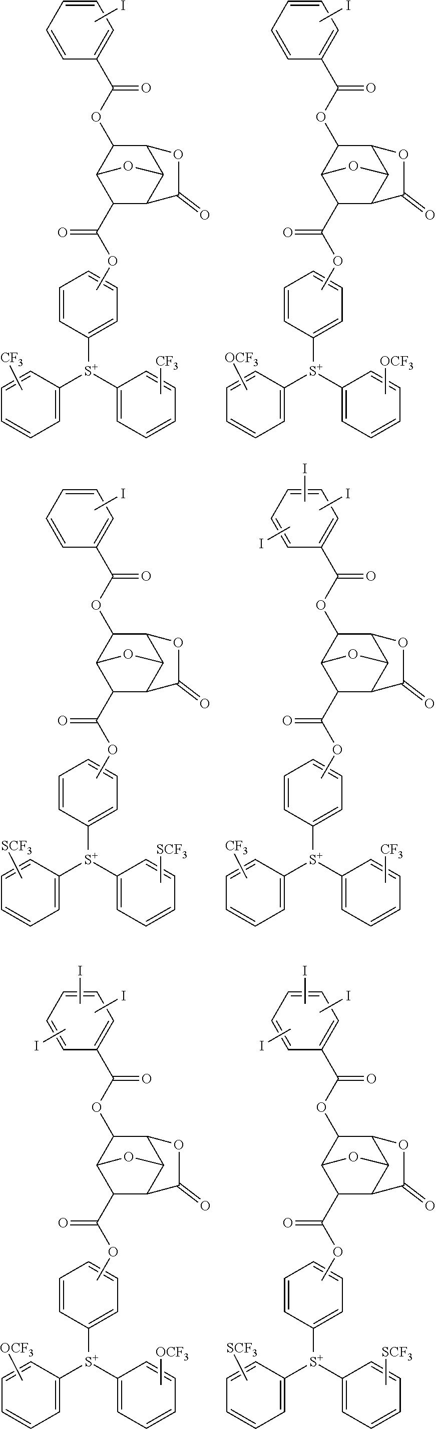

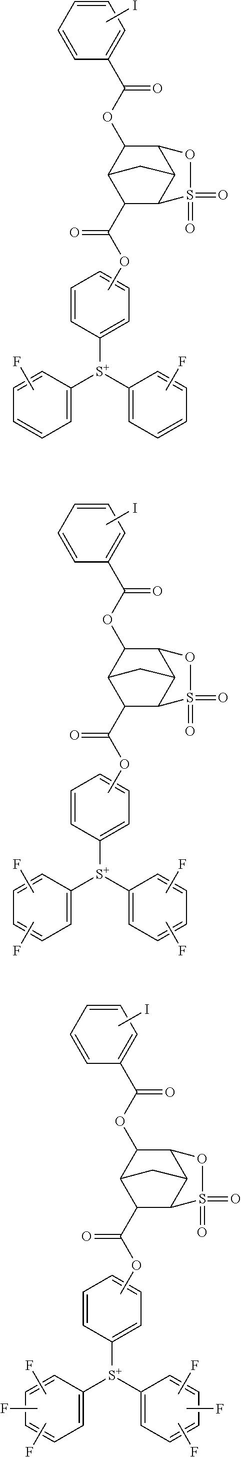

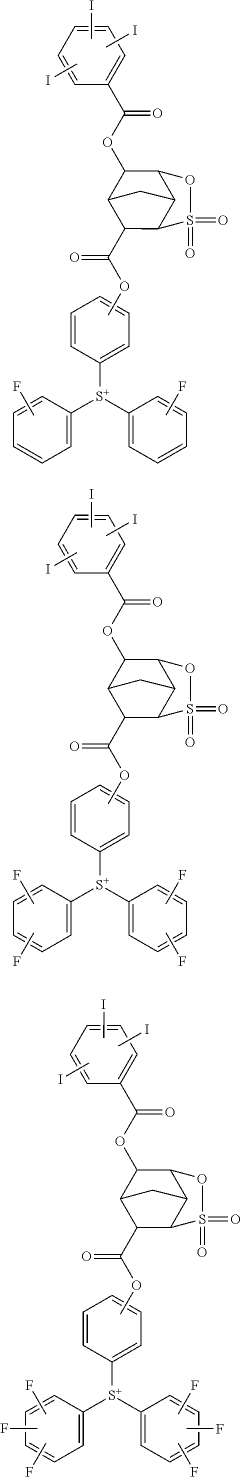

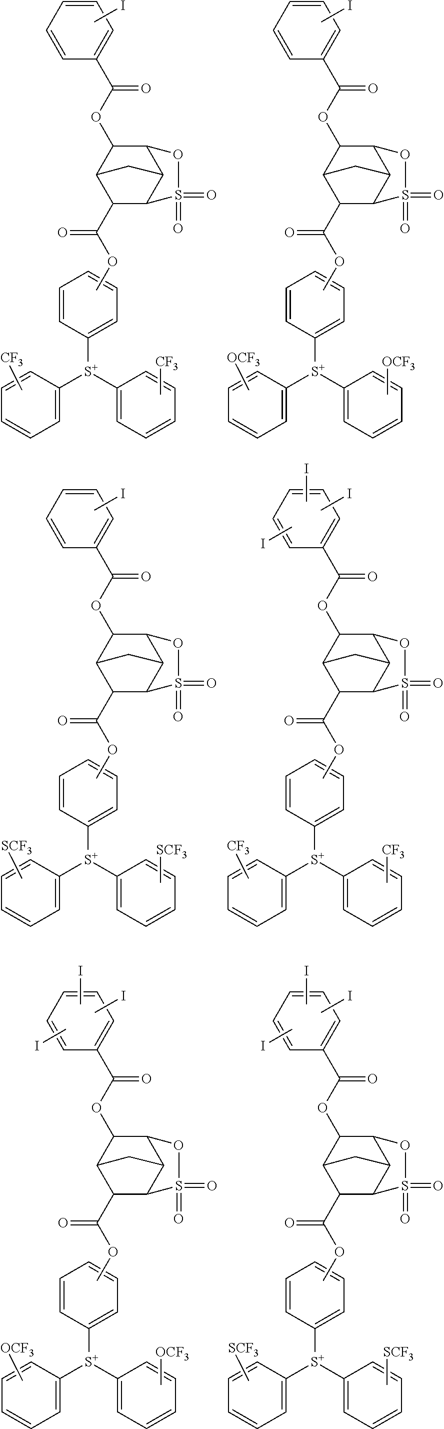

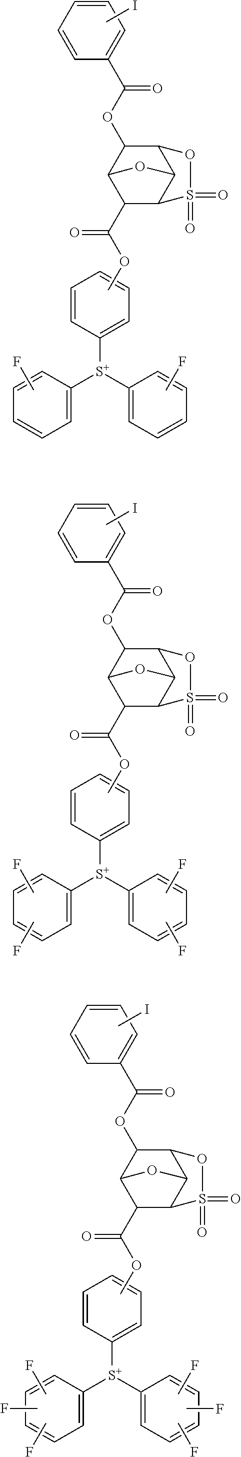

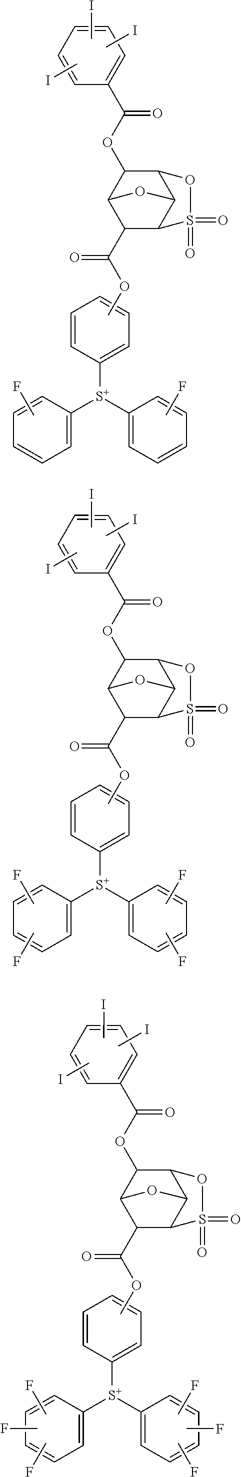

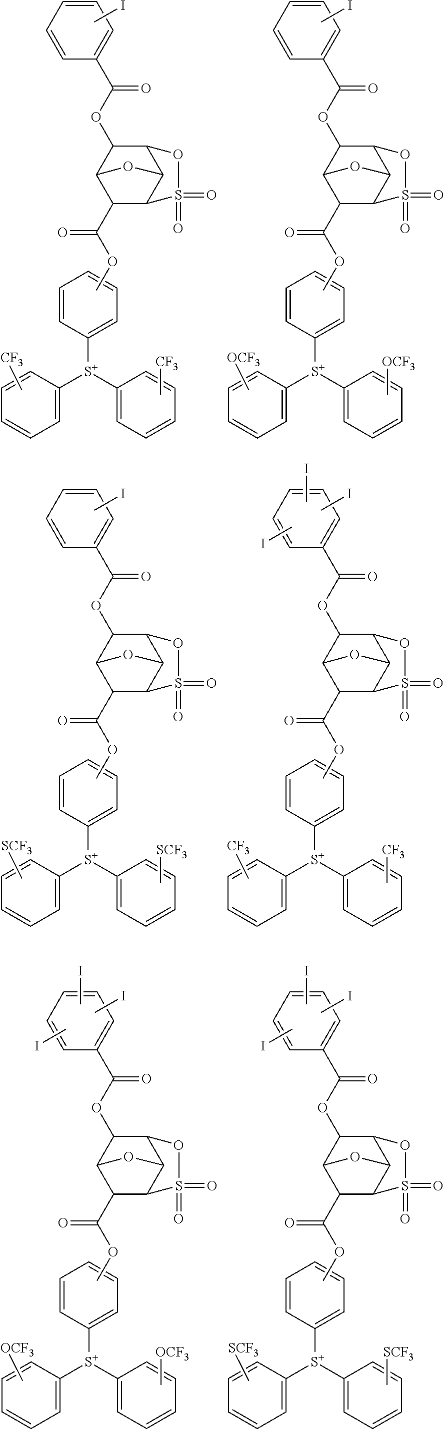

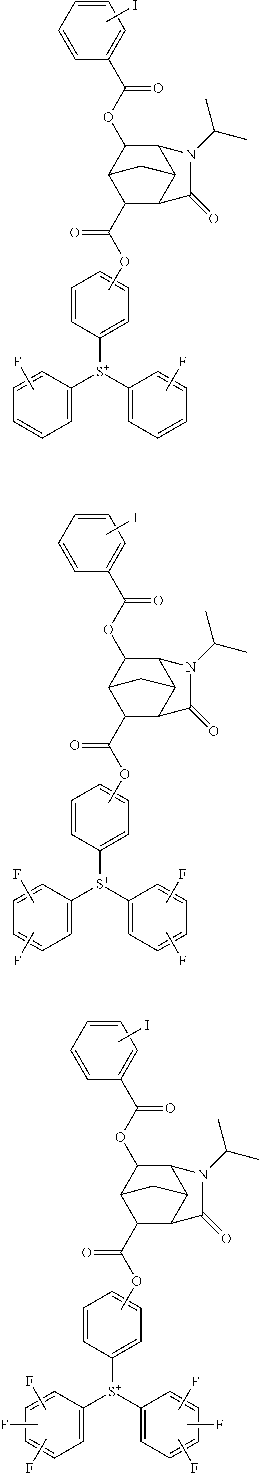

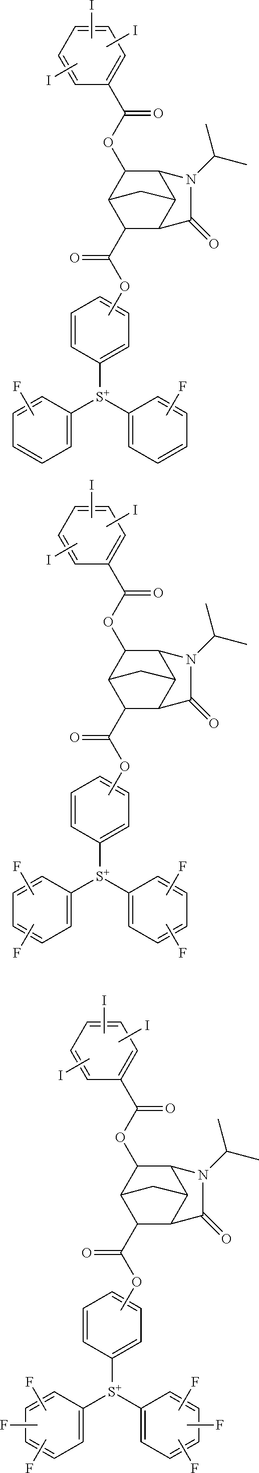

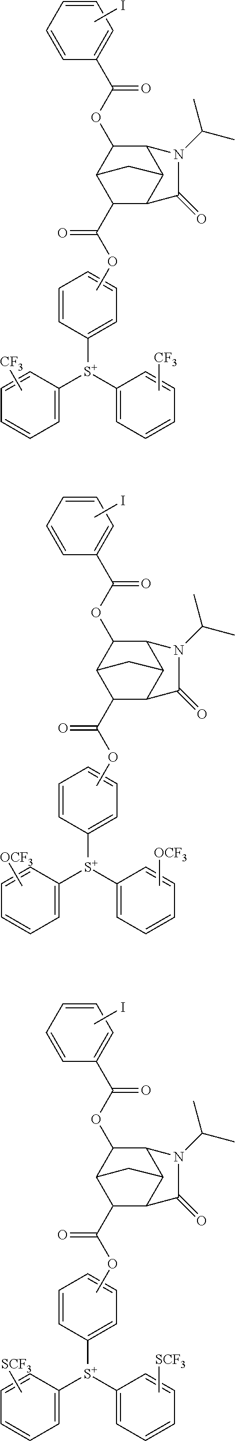

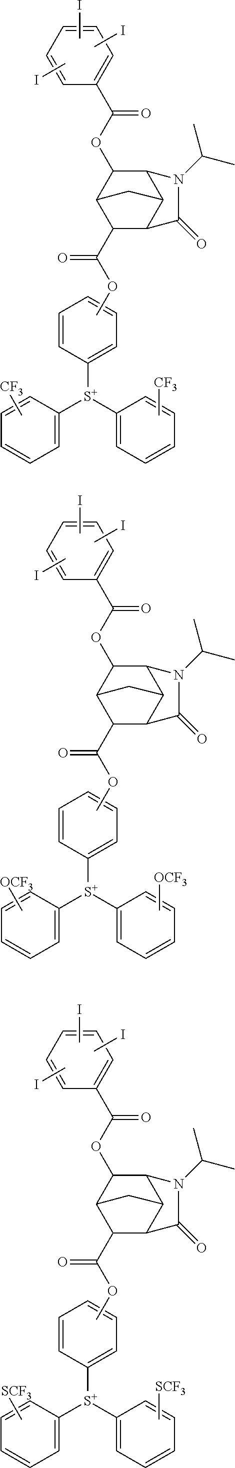

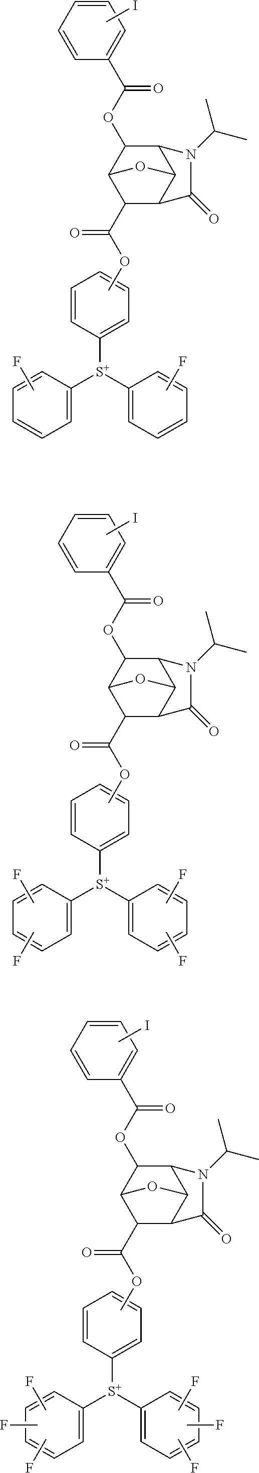

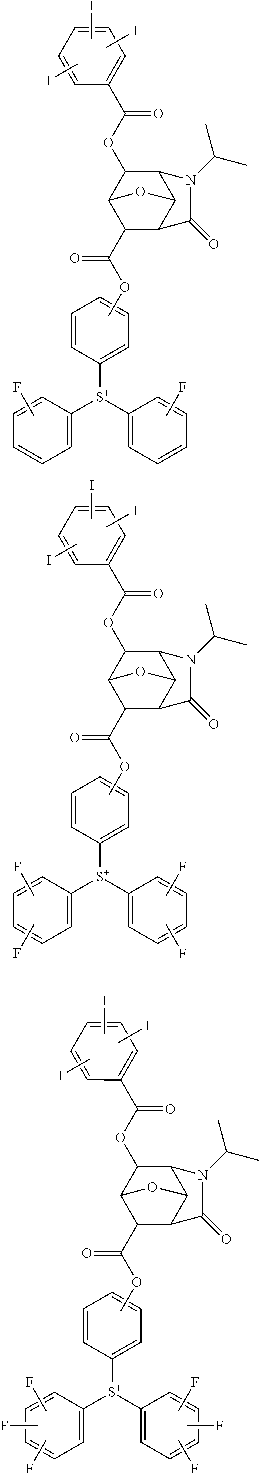

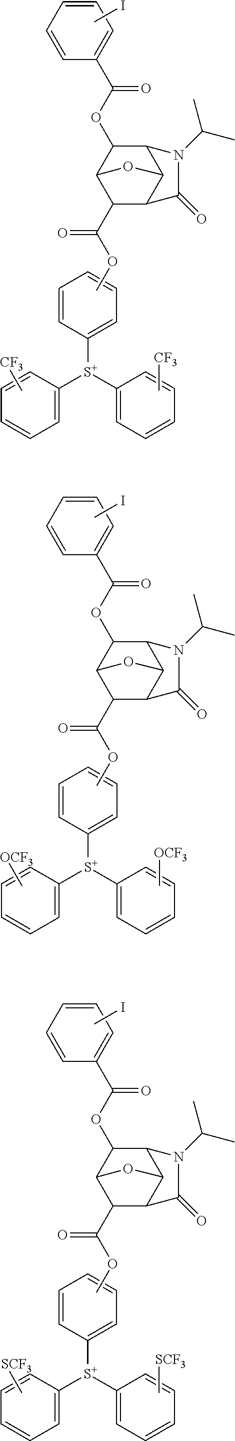

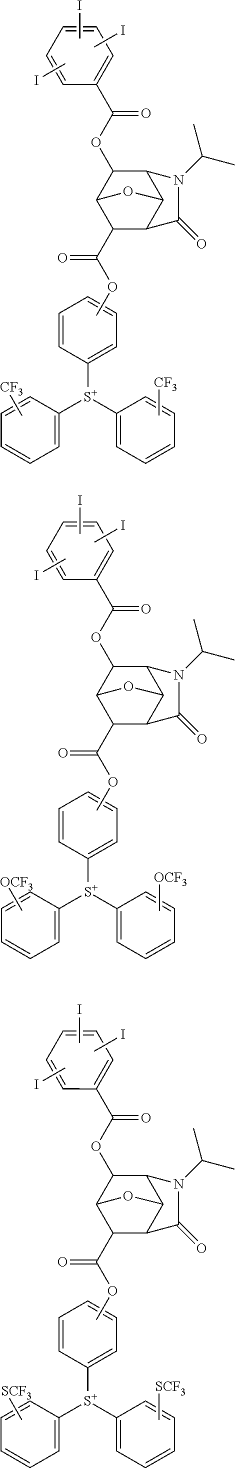

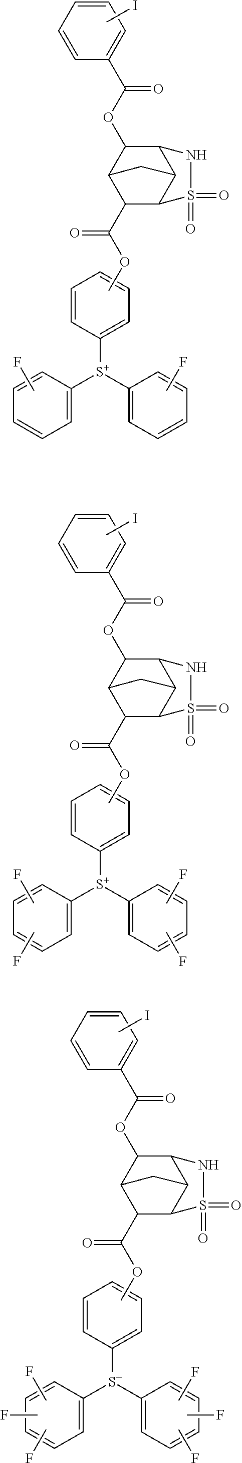

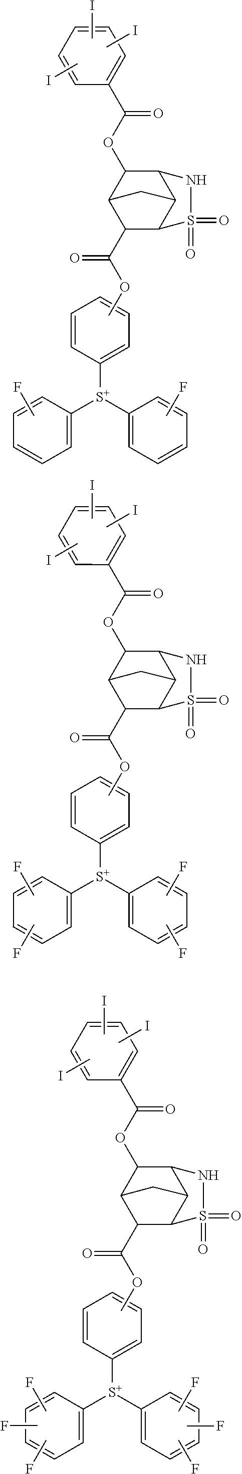

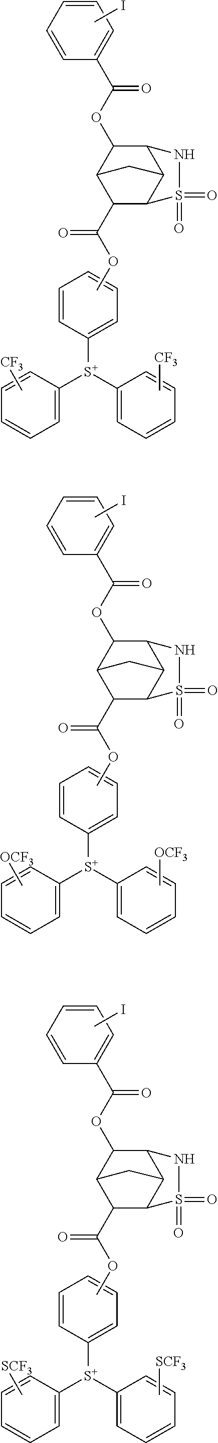

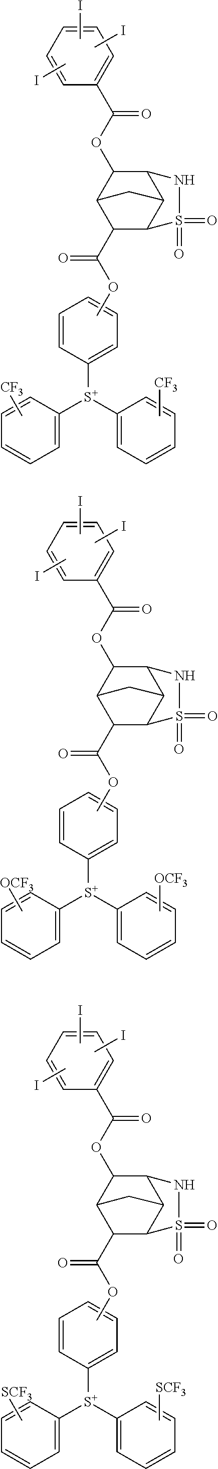

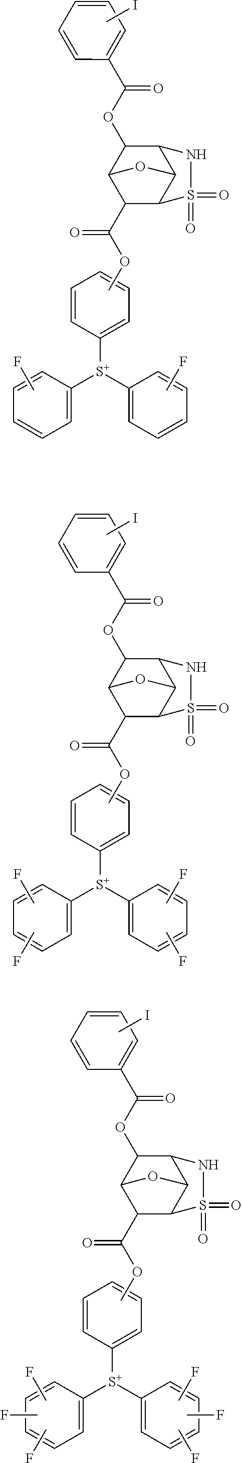

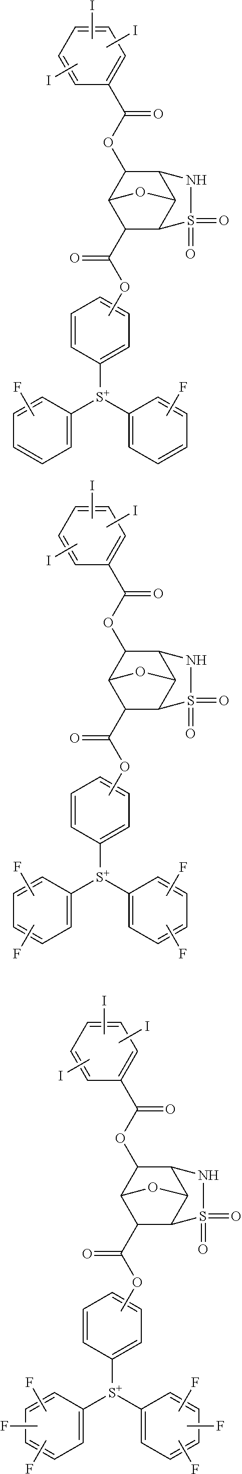

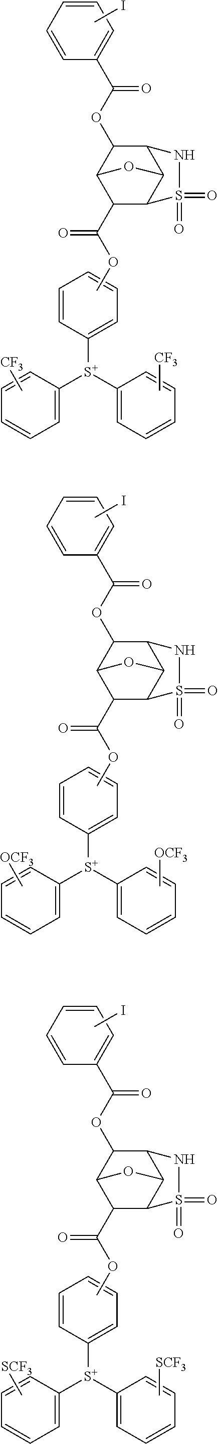

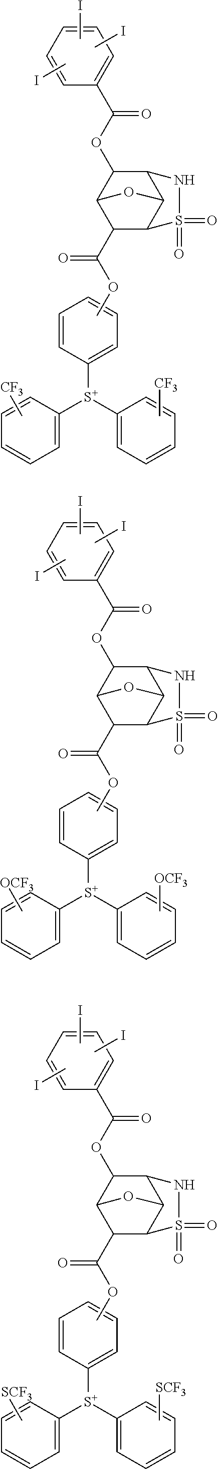

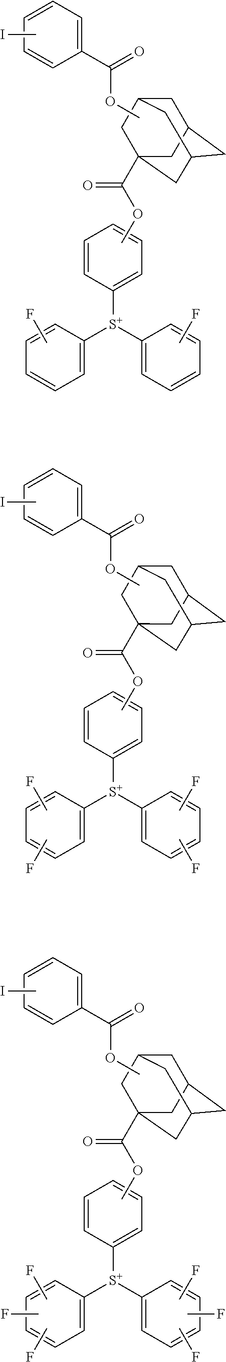

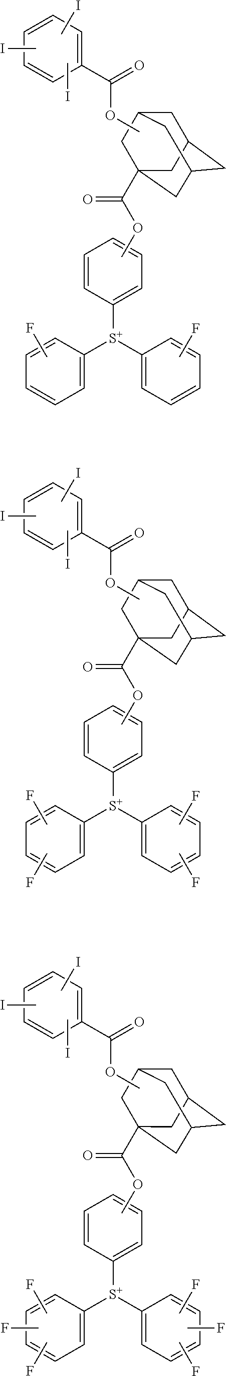

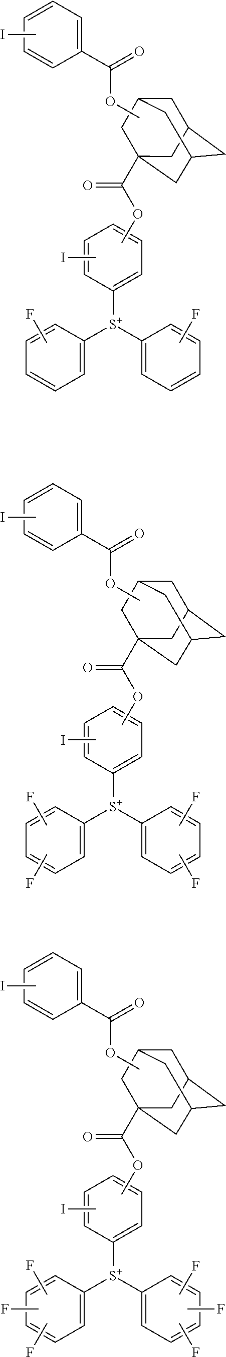

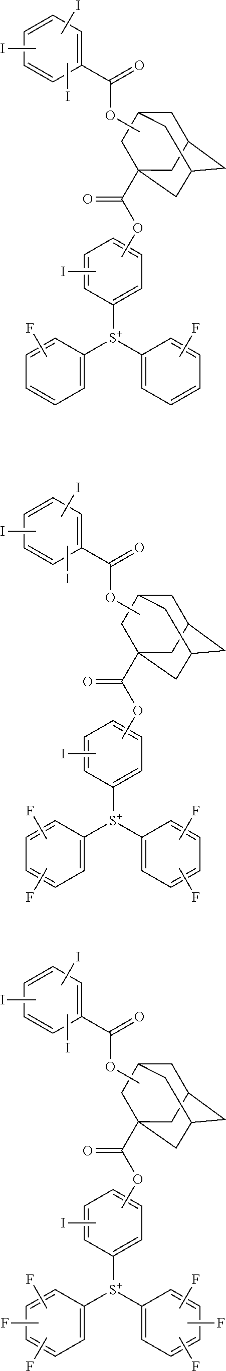

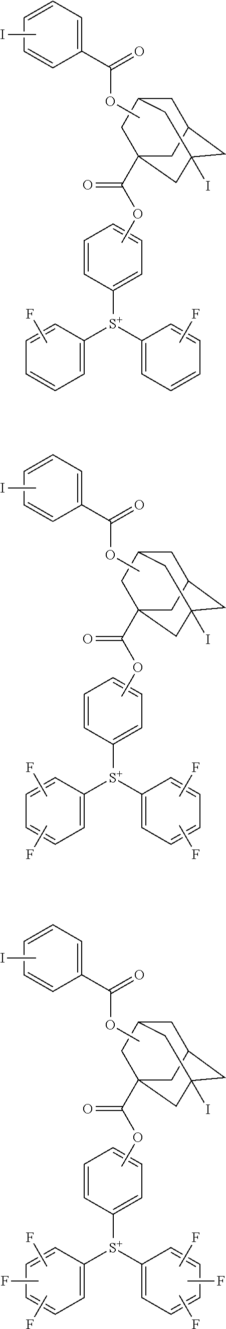

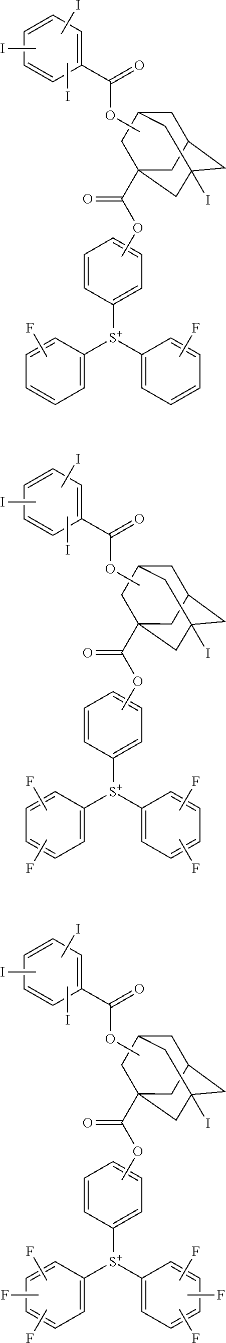

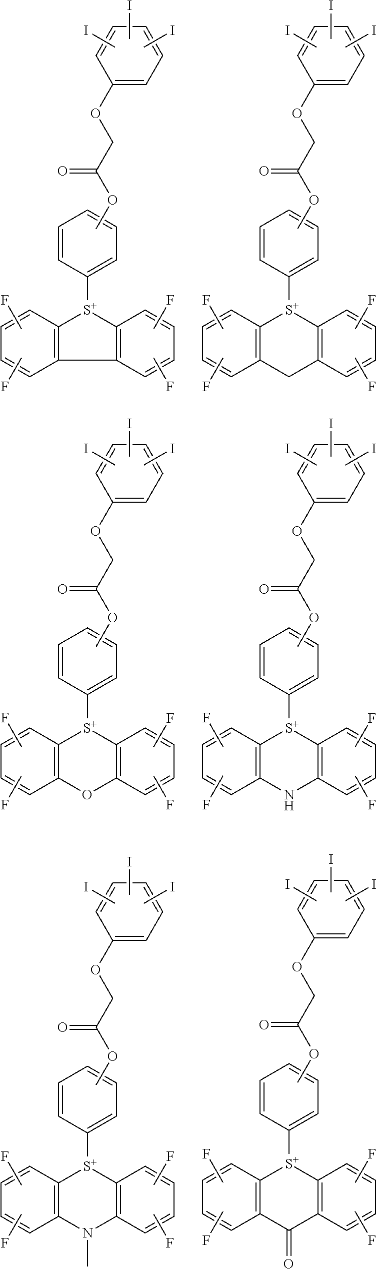

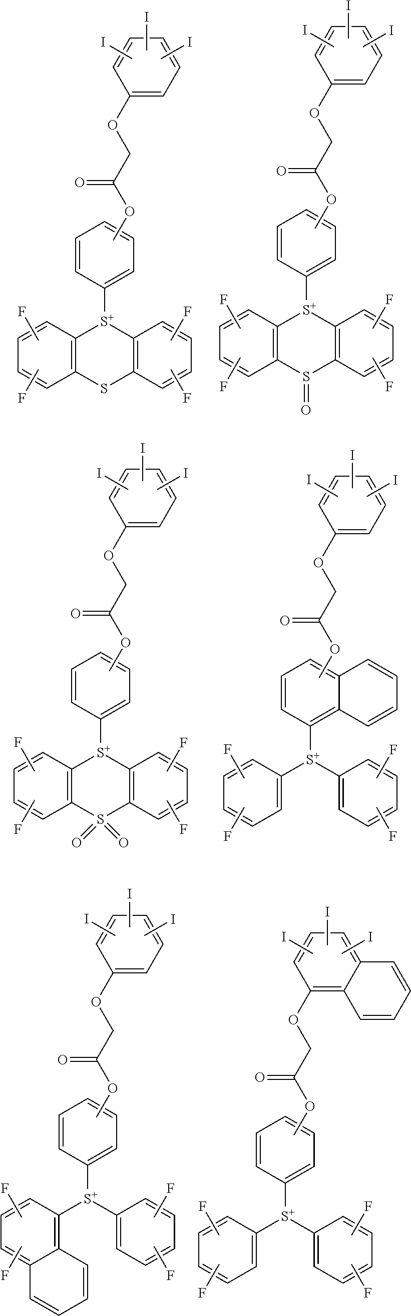

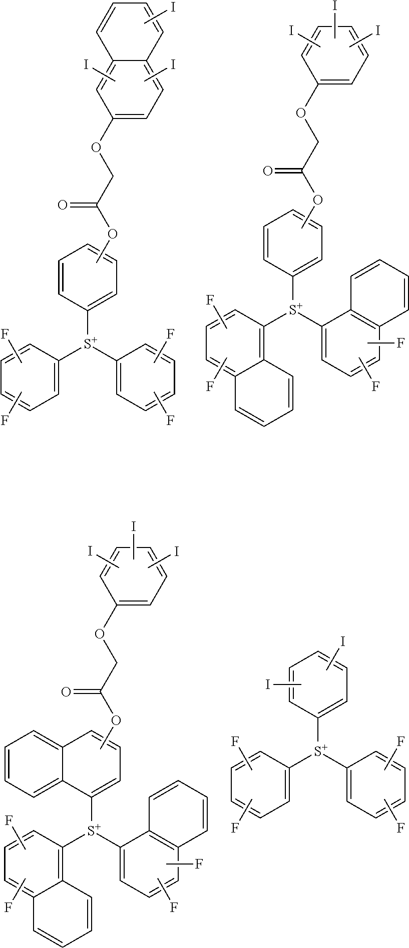

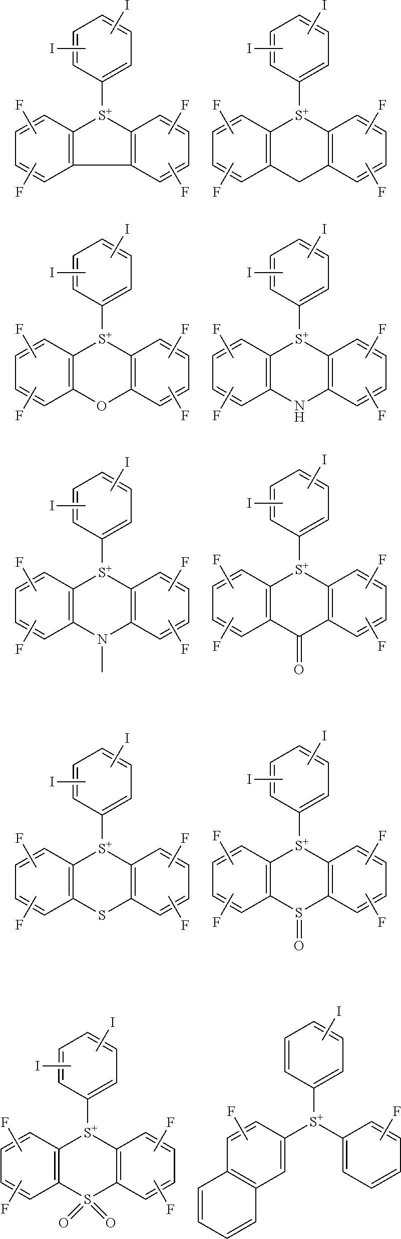

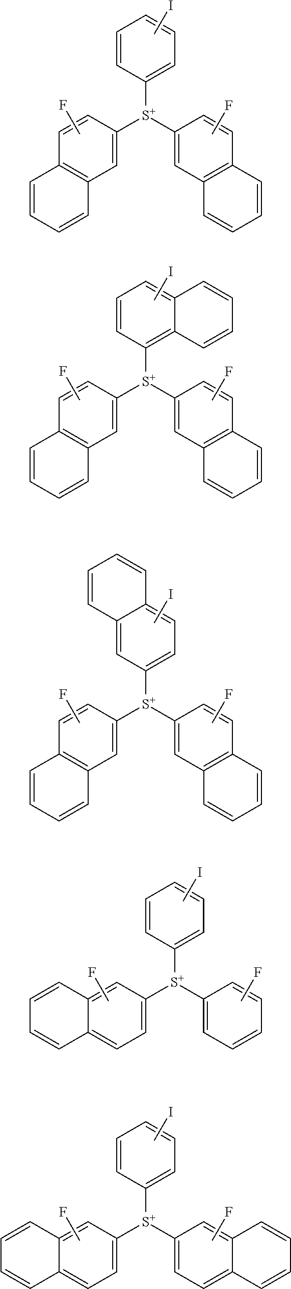

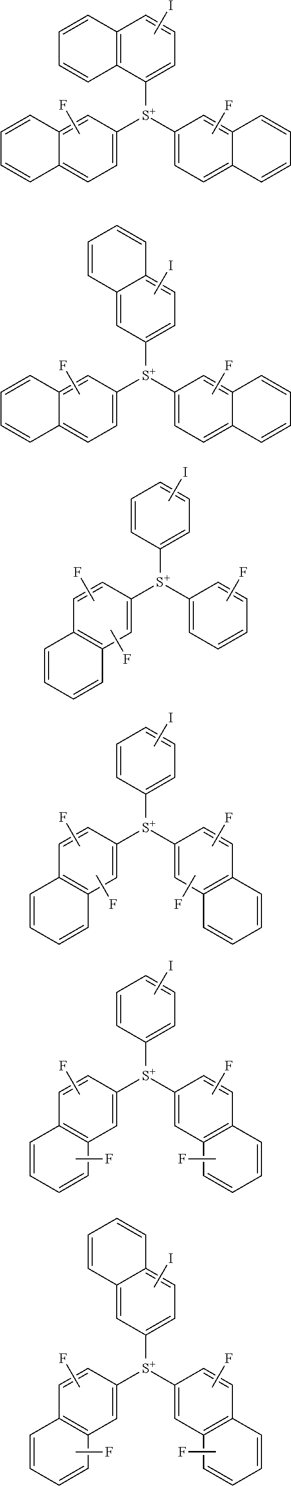

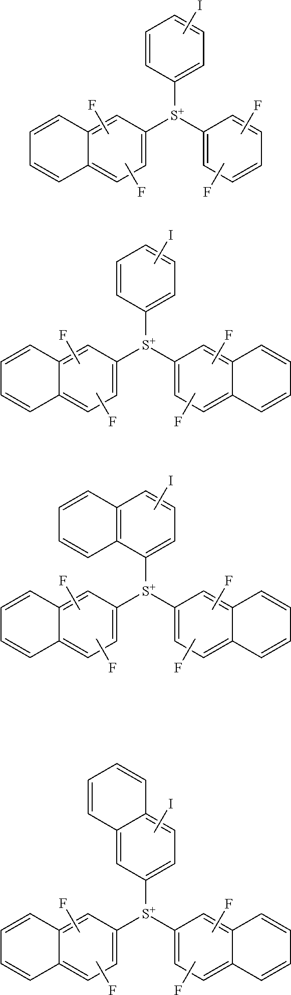

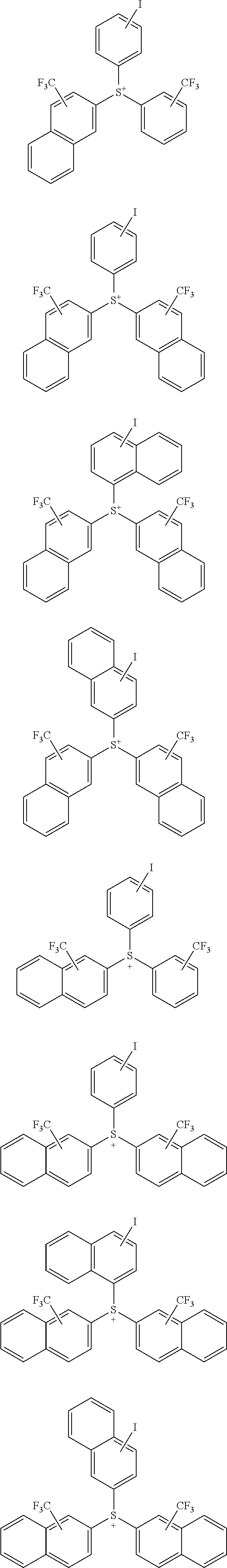

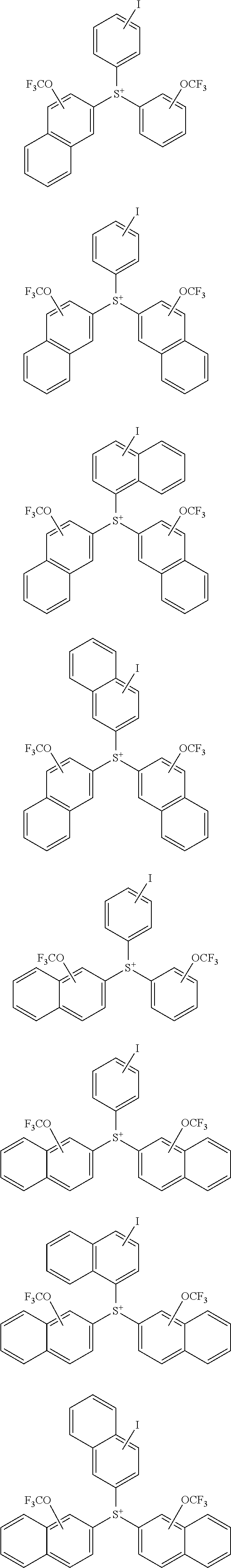

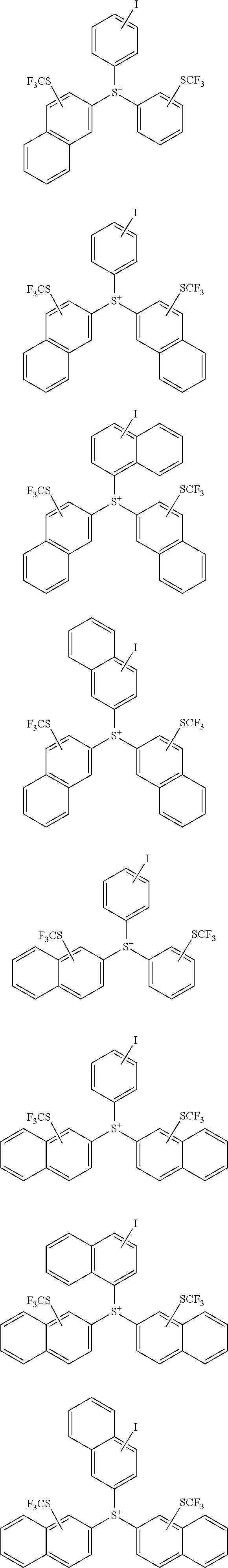

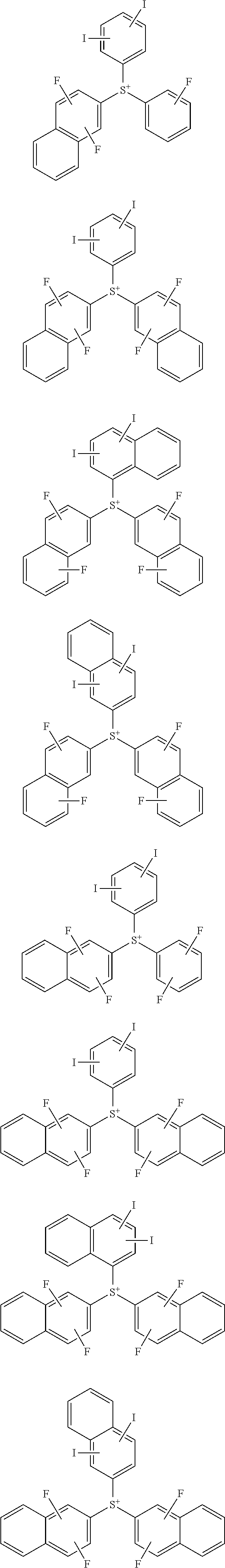

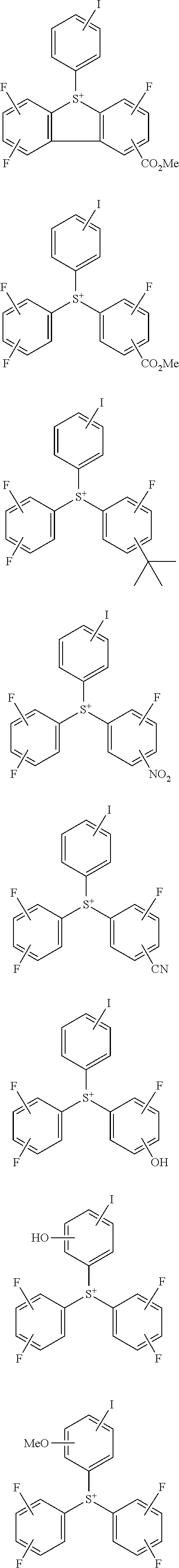

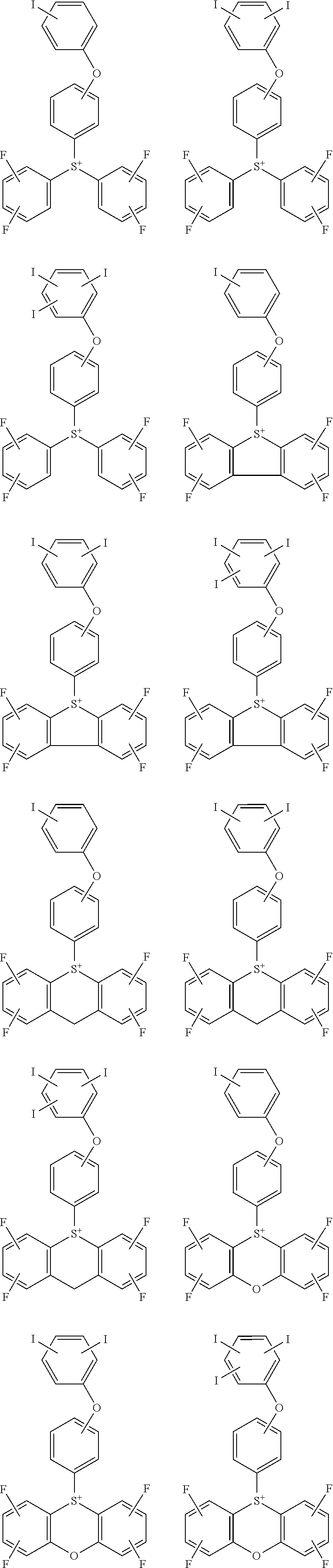

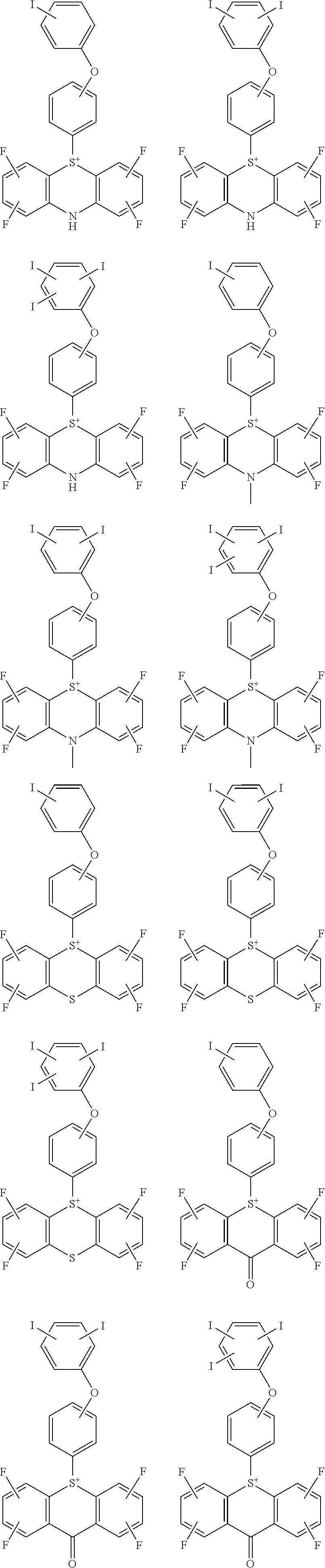

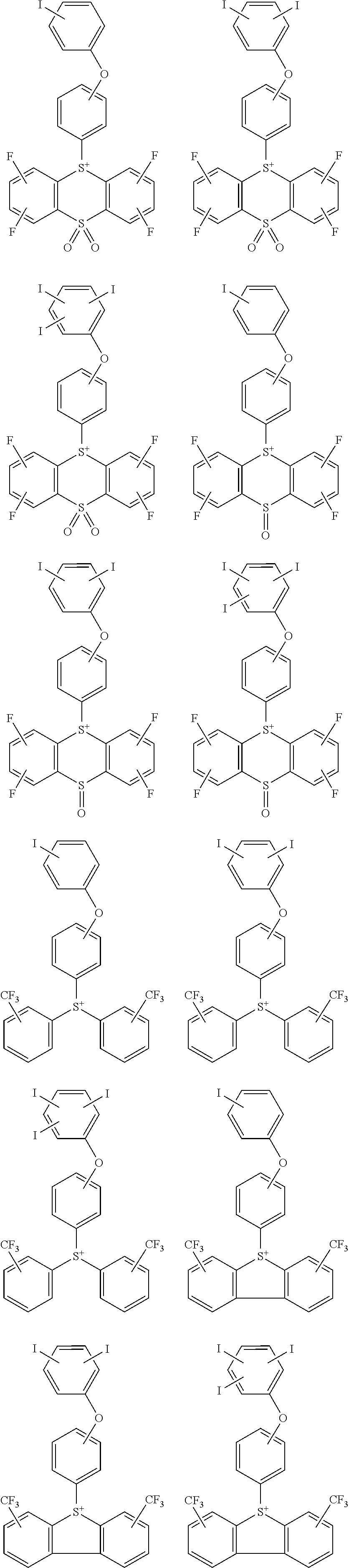

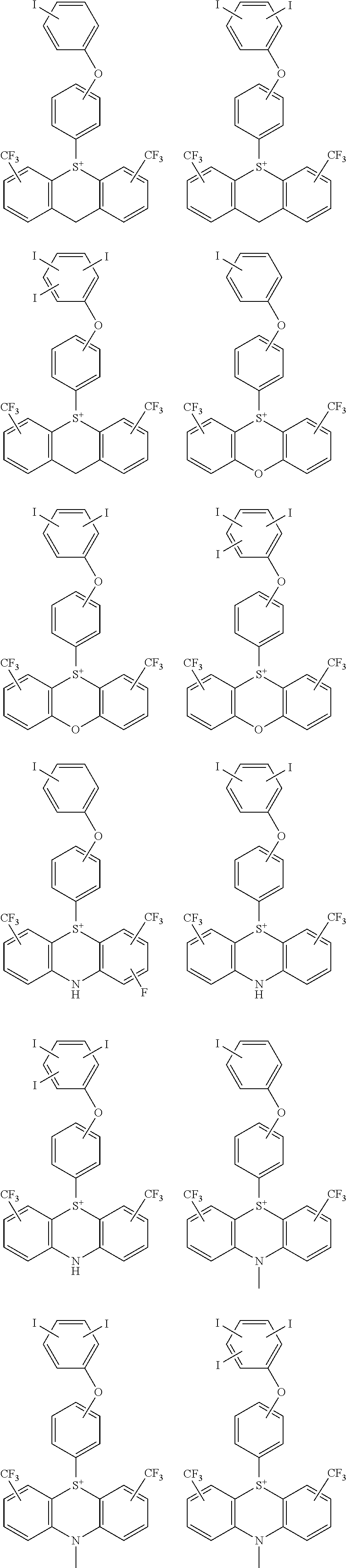

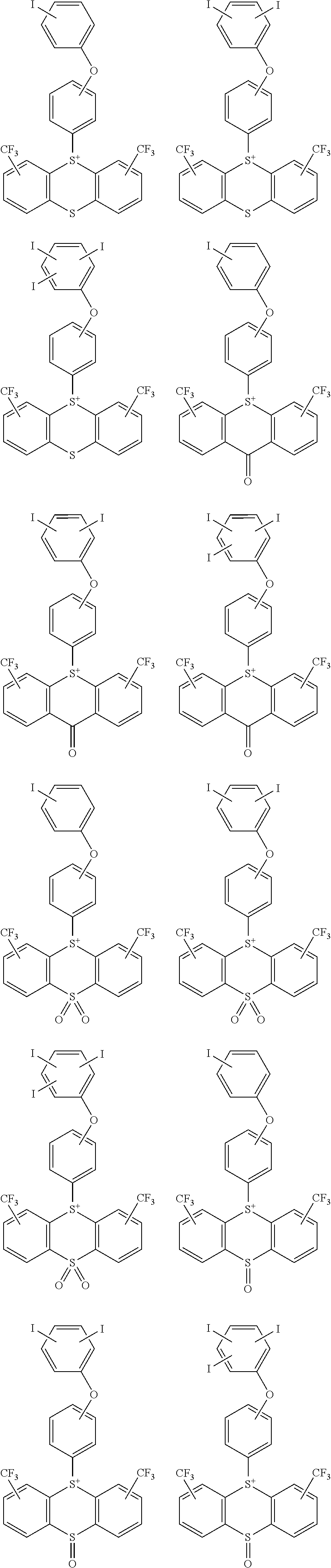

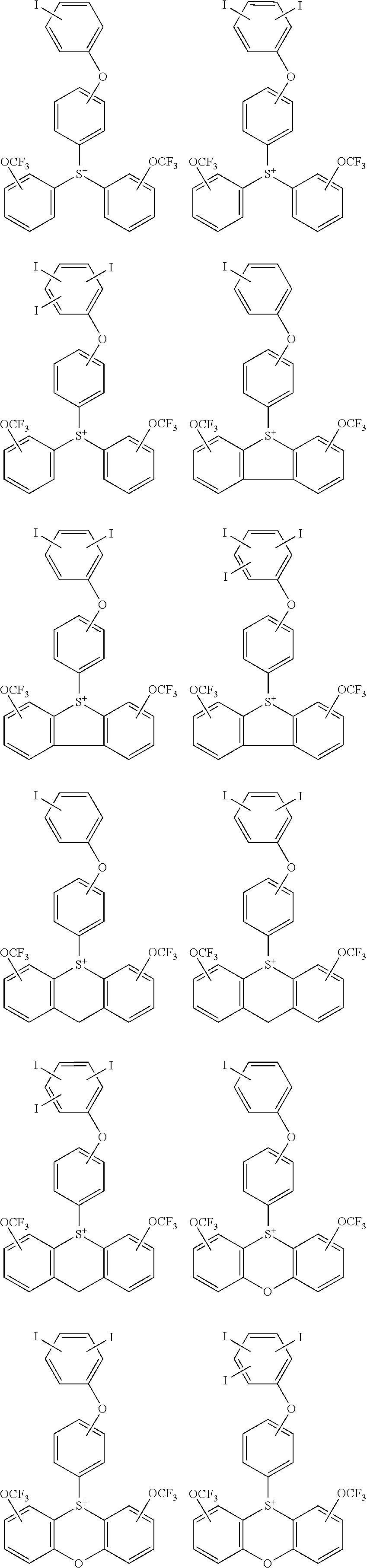

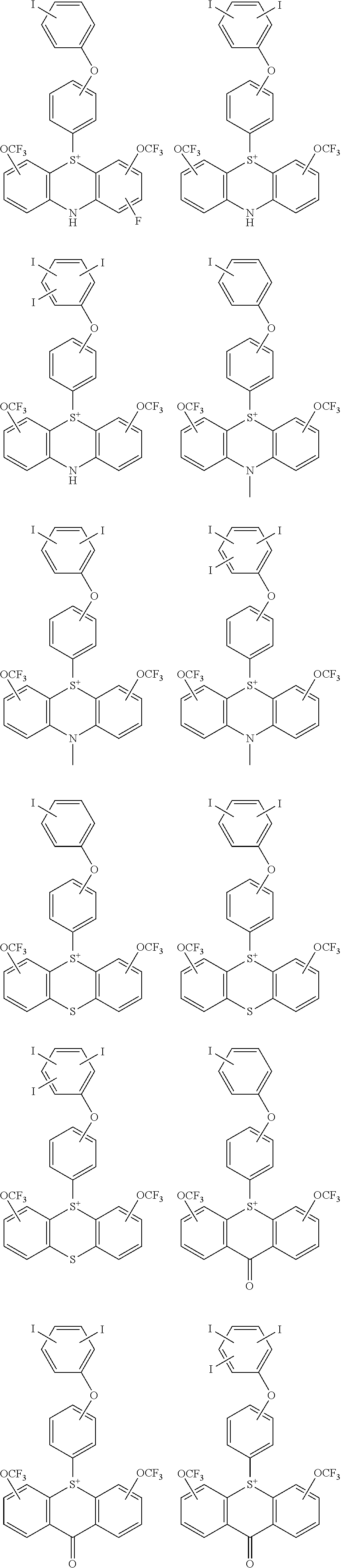

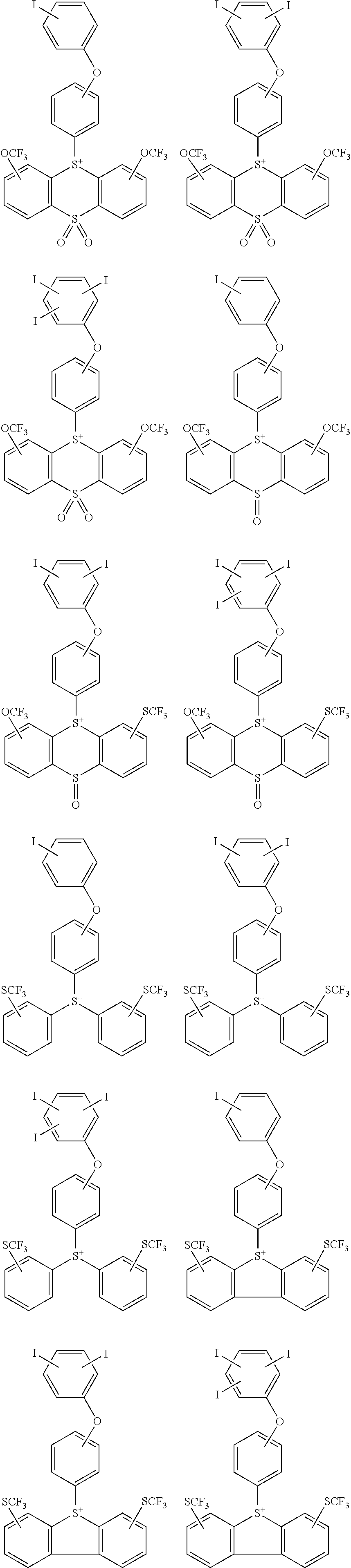

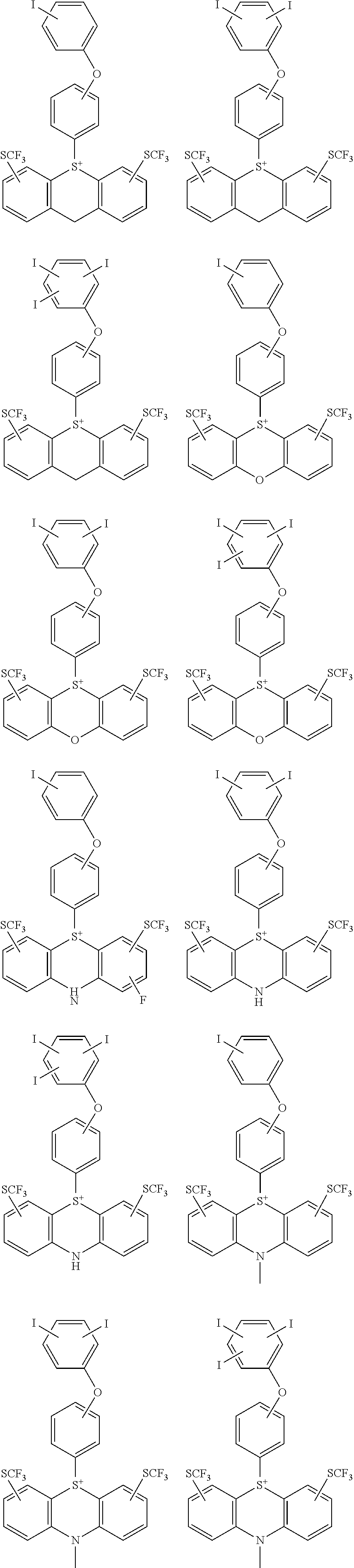

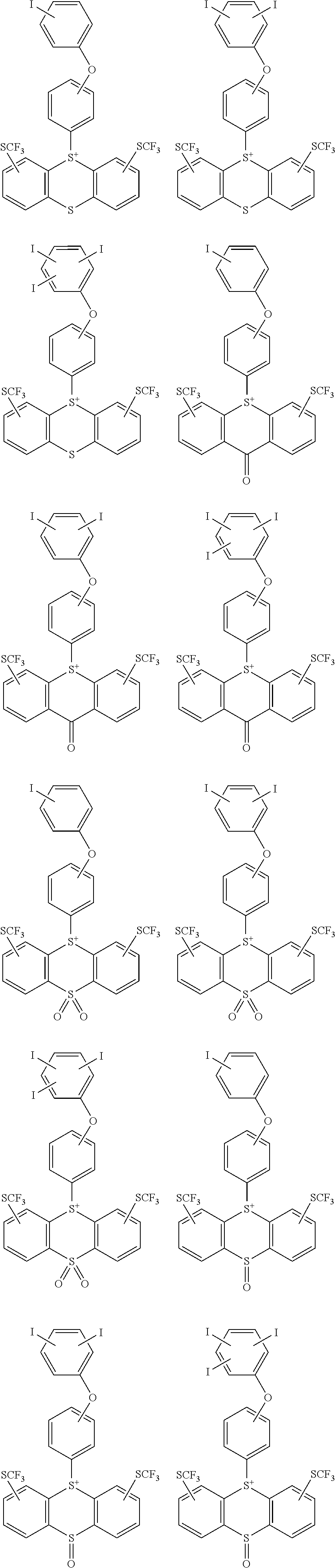

In formula (1), Xq⁻ is an anion. It is noted that the acid whose conjugated base is Xq⁻, i.e., XqH has a boiling point of lower than 165° C. and a molecular weight of 150 or less. Examples of XqH include formic acid, acetic acid, propionic acid, butyric acid, trifluoroacetic acid, 333-trifluoropropionic acid, pivalic acid, and nitric acid. XqH preferably has a boiling point of lower than 150° C. and a molecular weight of 120 or less.

Examples of the onium salt having formula (1) include arbitrary combinations of anions with cations, both as exemplified above.

The onium salt can be synthesized by a suitable method as described in JP-A 2020-91312, although the synthesis method is not limited thereto.

The inventive onium salt is advantageously used as a quencher in the resist composition. As used herein, the term “quencher” refers to a compound capable of trapping the strong acid generated by a PAG in the resist composition to prevent the acid from diffusing to the unexposed region and to assist in forming the desired pattern. As used herein, the “PAG” refers to a compound capable of generating a strong acid upon exposure to high-energy radiation, and the term “strong acid” refers to a compound having a sufficient acidity to induce deprotection reaction of an acid labile group. Since an acid such as acetic acid, nitric acid or trifluoroacetic acid generated from the inventive onium salt does not have an enough acidity to induce deprotection reaction of an acid labile group which is tertiary ester or tertiary ether, it is effective to separately add a PAG capable of generating a strong acid, i.e., α-fluorinated sulfonic acid, imide acid or methide acid, for the purpose of inducing deprotection reaction of an acid labile group, as will be described later. It is noted that the PAG capable of generating an α-fluorinated sulfonic acid, imide acid or methide acid may be of addition type or of polymer-bound type wherein the PAG is bound to a base polymer.

In a system where the inventive onium salt capable of generating the acid such as acetic acid, nitric acid or trifluoroacetic acid, and a PAG capable of generating an ultra-strong acid or perfluoroalkylsulfonic acid are co-present, the acid such as acetic acid, nitric acid or trifluoroacetic acid and the perfluoroalkylsulfonic acid generate upon light exposure. Since the PAG is not decomposed in its entirety, some PAG remains undecomposed nearby. If the onium salt capable of generating an acid such as acetic acid, nitric acid or trifluoroacetic acid and perfluoroalkylsulfonic acid are co-present at this point of time, first an ion exchange occurs between the perfluoroalkylsulfonic acid and the onium salt capable of generating an acid such as acetic acid, nitric acid or trifluoroacetic acid whereby an onium salt of perfluoroalkylsulfonic acid is generated and the acid such as acetic acid, nitric acid or trifluoroacetic acid is released. This is because the perfluoroalkylsulfonic acid salt having a high acid strength is more stable. On the other hand, where the perfluoroalkylsulfonic acid onium salt and the acid such as acetic acid, nitric acid or trifluoroacetic acid are co-present, no ion exchange occurs. Similar ion exchange takes place not only with the perfluoroalkylsulfonic acid, but also with an arene sulfonic acid, alkyl sulfonic acid, imide acid or methide acid having a higher acid strength than the acid such as acetic acid, nitric acid or trifluoroacetic acid generated by the inventive onium salt.

The onium salt of the invention is structurally characterized as consisting of a triaryl sulfonium cation containing iodine and fluorine and an anion in which an acid (XqH) as a conjugated base has a boiling point of lower than 165° C. and a molecular weight of 150 or less. In the triaryl sulfonium cation containing iodine and fluorine, iodine which is outstandingly absorptive to EUV of wavelength 13.5 nm generates secondary electrons during EUV exposure. Also, the electron withdrawing effect of fluorine serves to lower the energy level of the lowest unoccupied molecular orbital (LUMO) of the frontier orbital theory so that the cation is more likely to accept the generated secondary electrons, whereby the decomposition of the cation is promoted and the acid is effectively generated. On the other hand, since the weak acid generated from the onium salt of the invention has a low boiling point, it is presumed that a part of the generated acid volatilizes. This tendency is thought to be particularly noticeable in EUV lithography involving exposure under vacuum. The generated weak acid enters a developer, induces swelling of the resist film, and resultantly causes pattern collapse. On the other hand, in the onium salt, the week acid volatilizes, so that swelling can be reduced. This enables improvement of maximum resolution. The anion, which has a small molecular weight, thus is likely to be washed away with a developer. Therefore, a small amount of undissolved portions hardly exist at interfaces between exposed portions and unexposed portions after development, so that LWR is improved. The inventive resist composition having these characteristics is particularly effective in EUV lithography for forming line-and-space patterns with a pitch of 36 nm or less at which pattern collapse starts to become more noticeable. In these regions, high-level acid diffusion control and swelling reduction, and an enhanced dissolution contrast are important. The onium salt type quencher generates a weak non-sulfonic acid, and thus is low in acid diffusion, and low in swelling as described above. A polymer-bound acid generator obtained by incorporating a photoacid unit into a base polymer used is more preferred because acid diffusion can be controlled to a greater degree.

### Chemically Amplified Resist Composition

### (A) Quencher

Another embodiment of the invention is a chemically amplified resist composition essentially comprising (A) a quencher in the form of the onium salt having formula (1).

In the chemically amplified resist composition, the amount of quencher (A) is preferably 0.1 to 40 parts by weight, more preferably 0.5 to 30 parts by weight per 80 parts by weight of a base polymer to be described just below. As long as the amount of quencher (A) is in the range, good sensitivity and resolution are achievable and the risk of foreign matter being formed after development or during stripping of resist film is avoided. The quencher (A) may be used alone or in admixture.

### (B) Base Polymer

The chemically amplified resist composition may comprise a base polymer as component (B). The base polymer (B) contains at least one selected from repeat units having the formula (a1), which are also referred to as repeat units (a1), and repeat units having the formula (a2), which are also referred to as repeat units (a2).

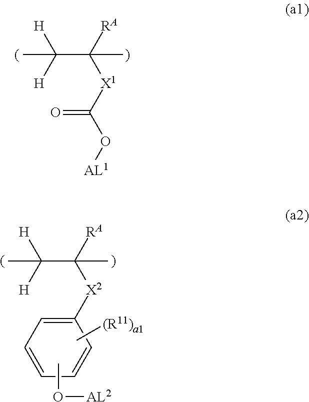

In formulae (a1) to (a2), R^{A} is each independently hydrogen, fluorine, methyl or trifluoromethyl.

In formula (a1), X¹ is a single bond, phenylene group, naphthylene group, *—C(═O)—O—X¹¹— or *—C(═O)—NH—X¹¹—, the phenylene group or naphthylene group may be substituted with hydroxy group, nitro group, cyano group, a C₁-C₁₀ saturated hydrocarbyl group which may contain fluorine, a C₁-C₁₀ saturated hydrocarbyloxy group which may contain fluorine, or halogen. X¹¹ is a C₁-C₁₀ saturated hydrocarbylene group, phenylene group, or naphthylene group, and the saturated hydrocarbylene group may contain a hydroxy group, ether bond, ester bond or lactone ring. The asterisk (*) designates a point of attachment to the carbon atom in the backbone.

In formula (a2), X² is a single bond, *—C(═O)—O— or *—C(═O)—NH—. The asterisk (*) designates a point of attachment to the carbon atom in the backbone. R¹¹ is halogen, cyano group, hydroxy group, nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. A plurality of R¹¹ may be identical or different when a1 is 2, 3 or 4. The subscript a1 is 0, 1, 2, 3 or 4, preferably 0 or 1.

In formulae (a1) and (a2), AL¹ and AL² are each independently an acid labile group. Examples of the acid labile groups are as described in JP-A 2013-80033 and JP-A 2013-83821.

Typical of the acid labile group are groups of the following formulae (AL-1) to (AL-3).

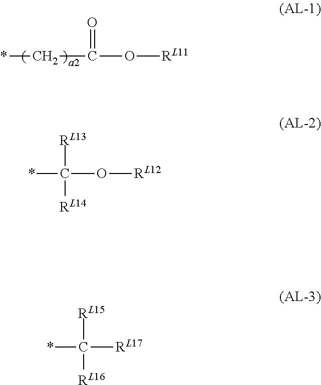

Herein the broken line denotes a point of attachment.

In formulae (AL-1) and (AL-2), R^{L11} and R^{L12} are each independently a C₁-C₄₀ hydrocarbyl group which may contain a heteroatom such oxygen, sulfur, nitrogen or fluorine. The hydrocarbyl group may be saturated or unsaturated, and may be straight, branched, or cyclic. The hydrocarbyl group is preferably a C₁-C₂₀ hydrocarbyl group.

In formula (AL-1), a2 is an integer of 0 to 10, preferably 1, 2, 3, 4 or 5.

In formula (AL-2), R^{L13} and R^{L14} are each independently hydrogen or a C₁-C₂₀ hydrocarbyl group which may contain a heteroatom such as oxygen, sulfur, nitrogen, or fluorine. The hydrocarbyl group may be saturated or unsaturated, and may be straight, branched, or cyclic. The hydrocarbyl group is preferably a C₁-C₂₀ hydrocarbyl group. Any two of R^{L12}, R^{L13} and R^{L14} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L15}, R^{L16} and R^{L17} are each independently a C₁-C₂₀ hydrocarbyl group which may contain a heteroatom such as oxygen, sulfur, nitrogen or fluorine. The hydrocarbyl group may be saturated or unsaturated, and may be straight, branched, or cyclic. The hydrocarbyl group is preferably a C₁-C₂₀ hydrocarbyl group. Any two of R^{L15}, R^{L16} and R^{L17} may bond together to form a ring, typically an alicyclic ring, with a carbon atom to which they are bonded, the ring containing 3 to 20 carbon atoms. The ring preferably contains 4 to 16 carbon atoms and is typically alicyclic.

Examples of repeat unit (a1) are shown below, but not limited thereto. Herein R^{A} and AL¹ are as defined above.

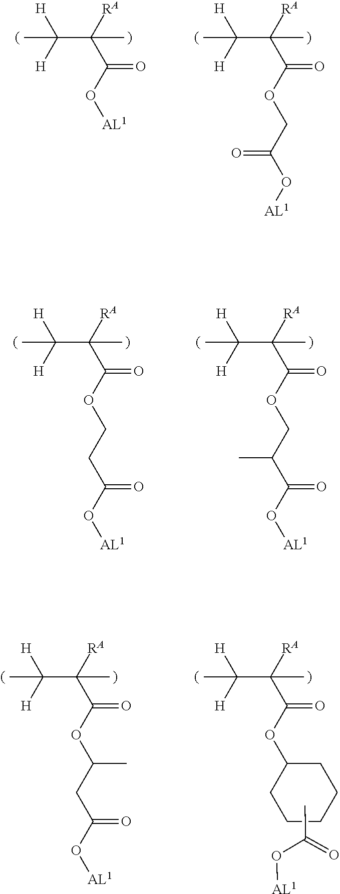

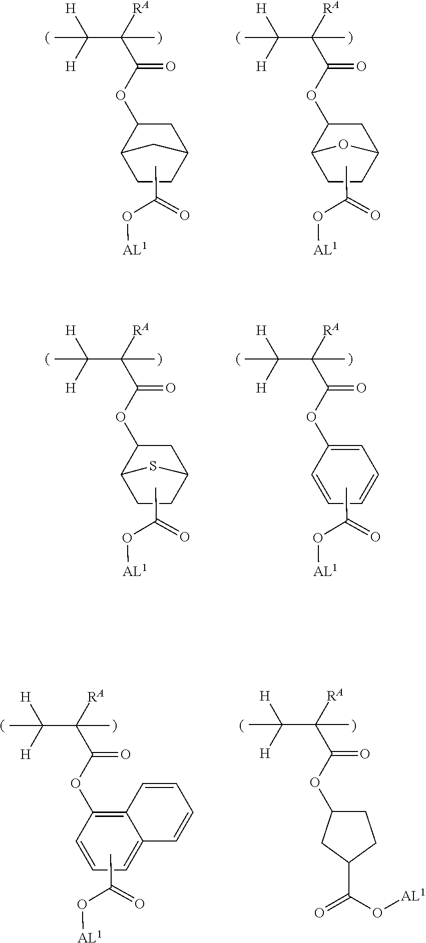

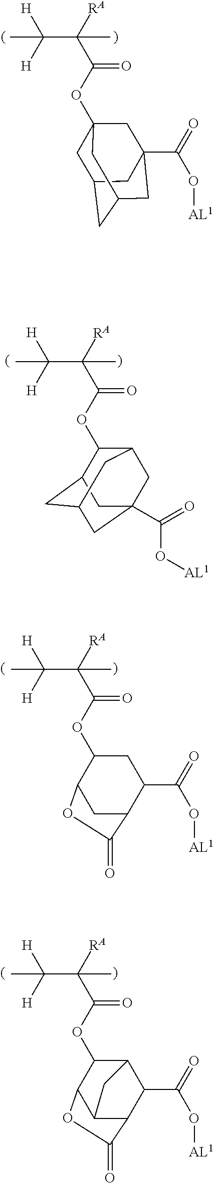

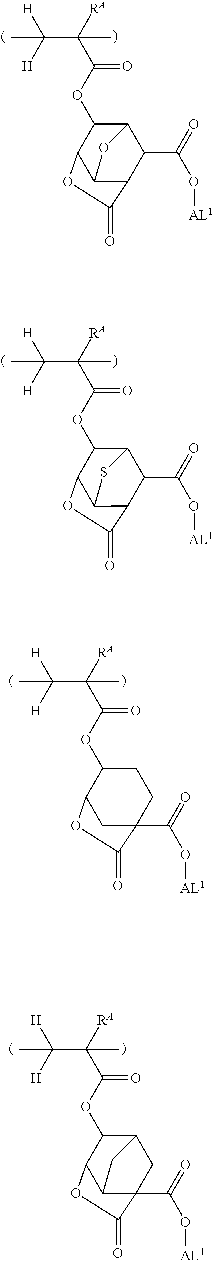

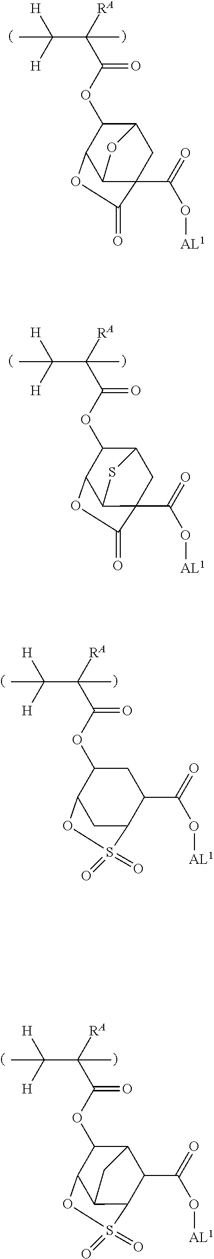

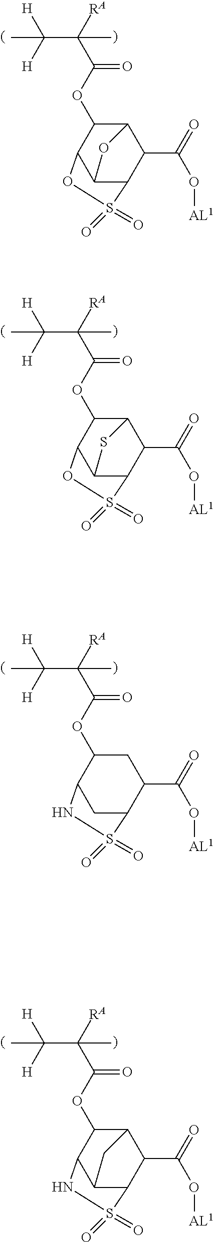

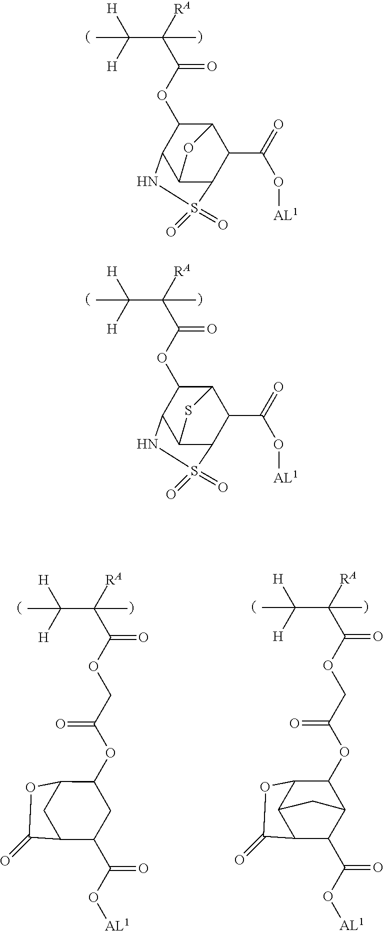

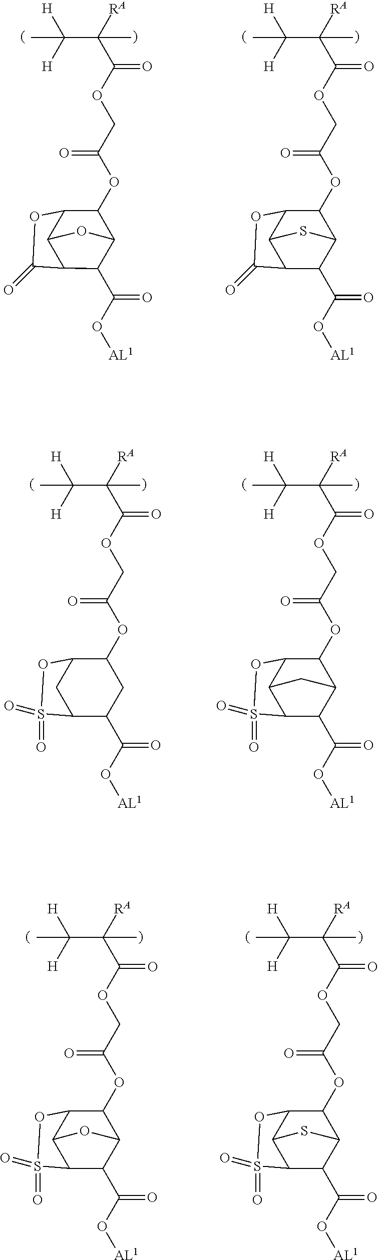

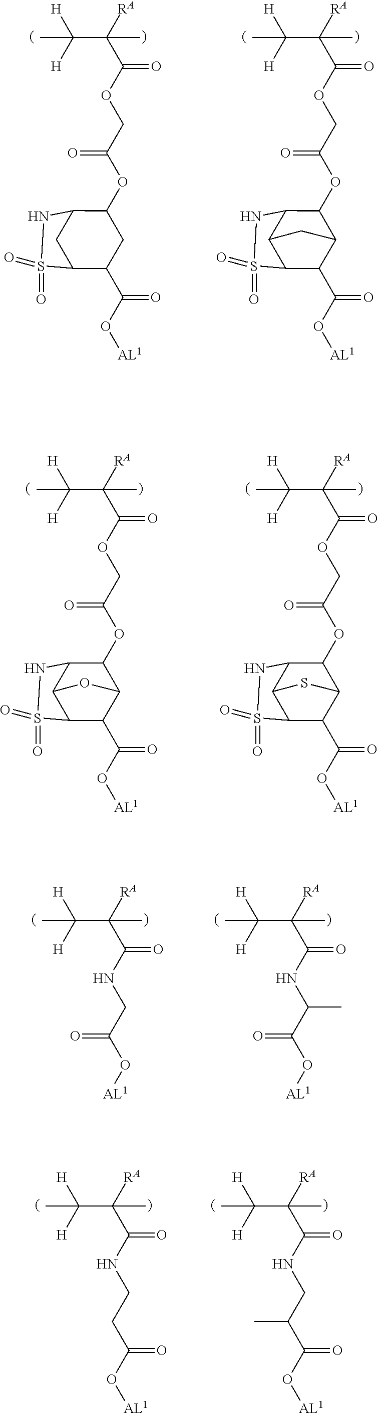

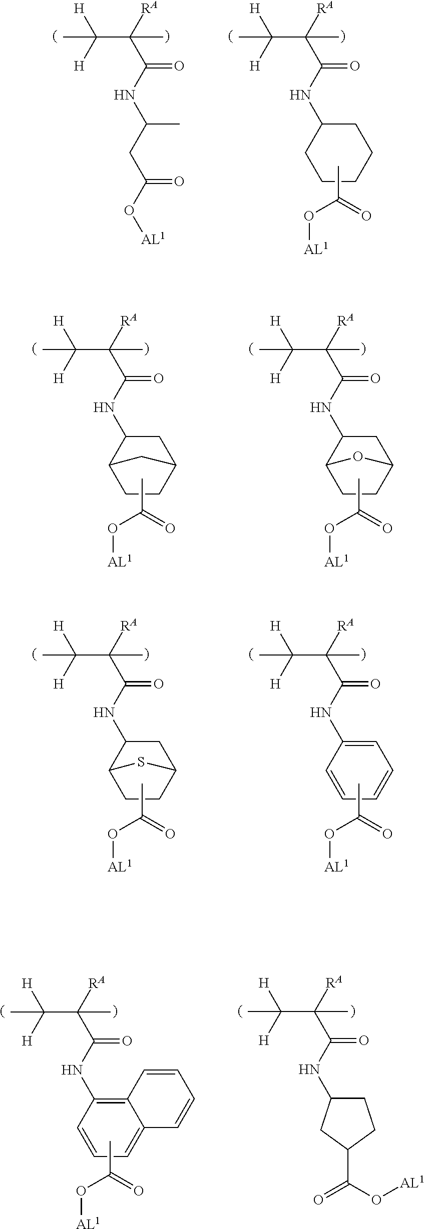

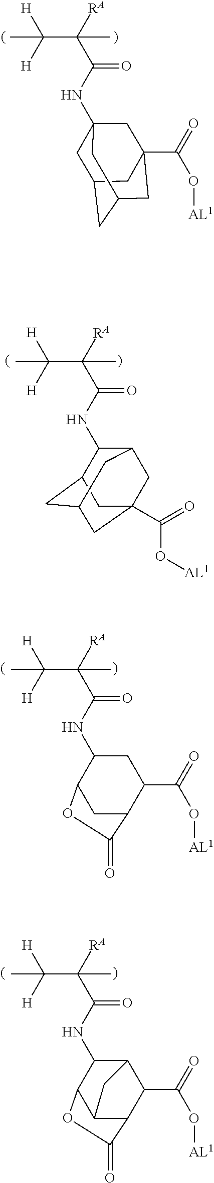

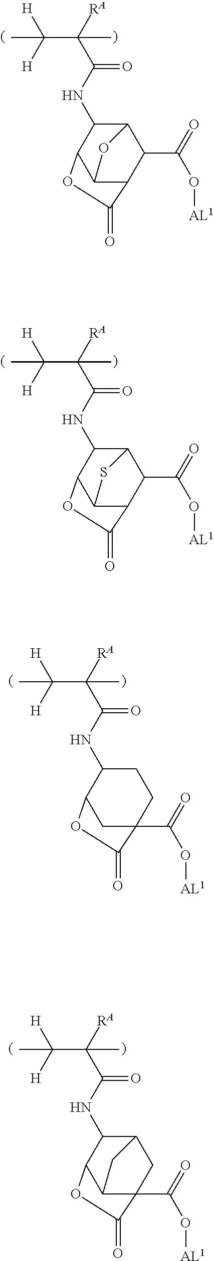

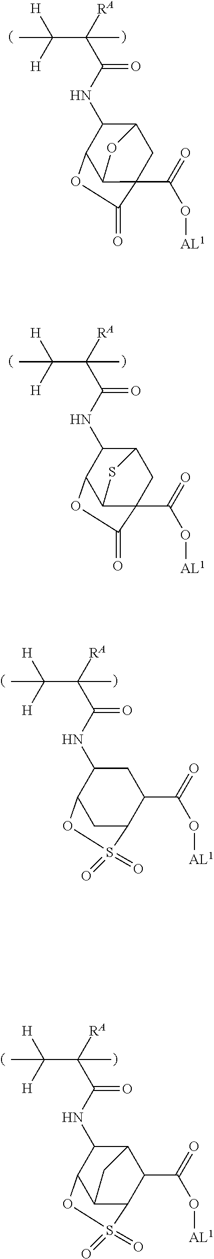

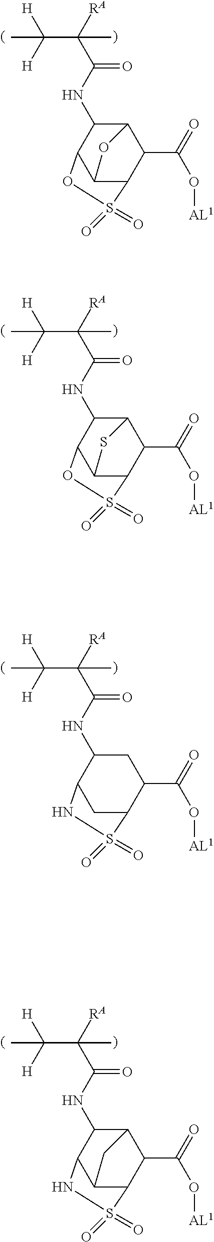

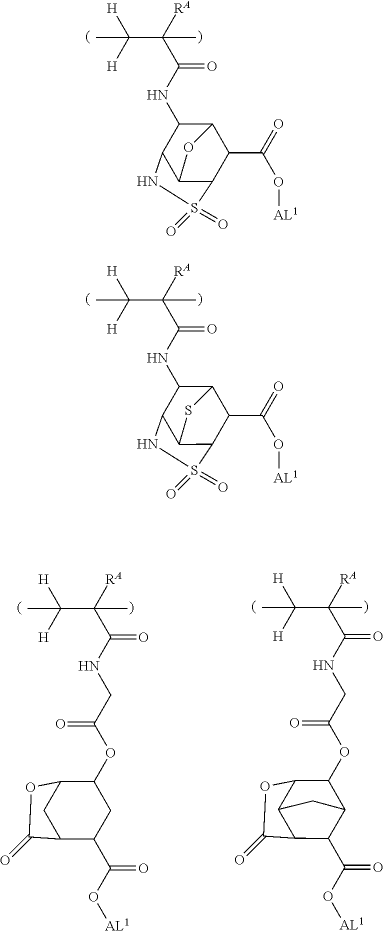

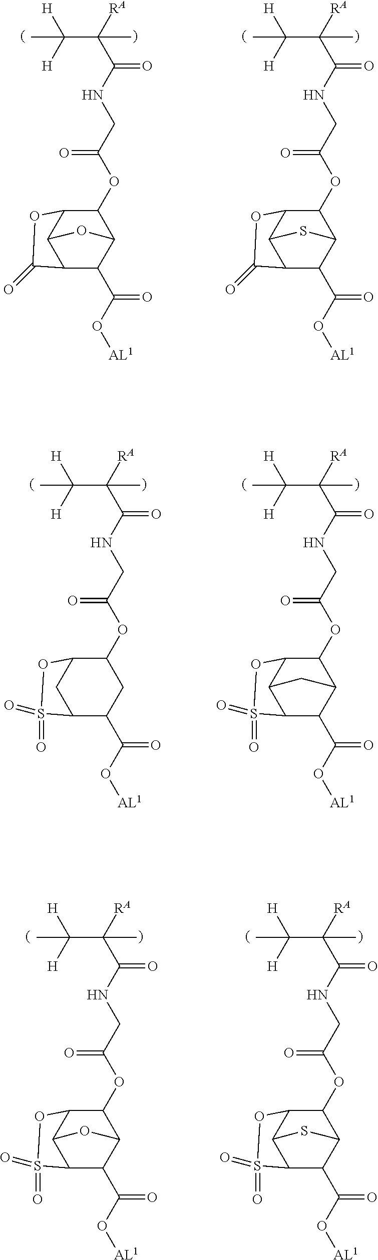

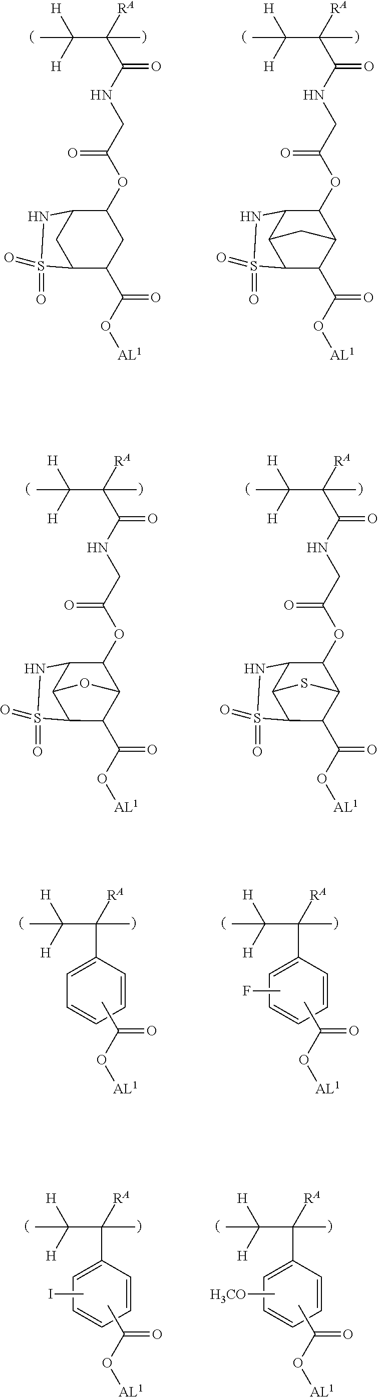

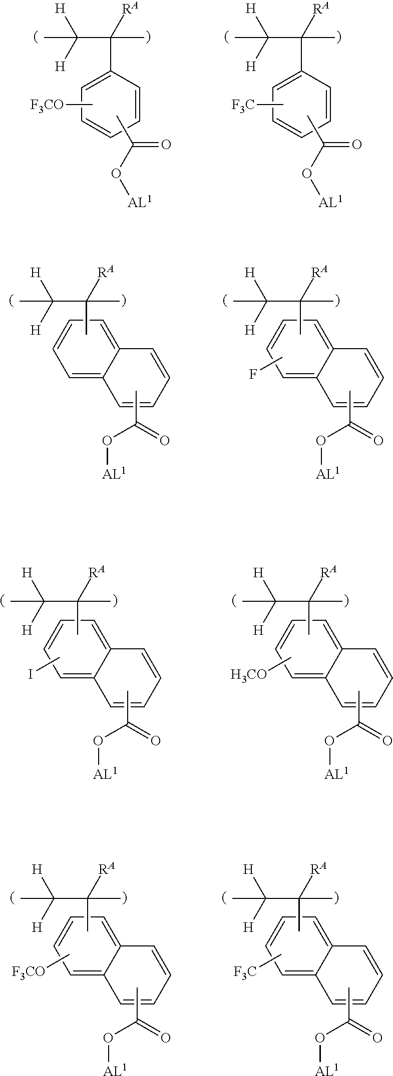

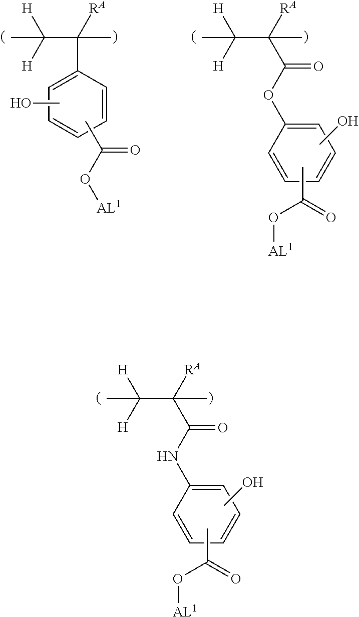

Examples of repeat unit (a2) are shown below, but not limited thereto. Herein R^{A} and AL² are as defined above.

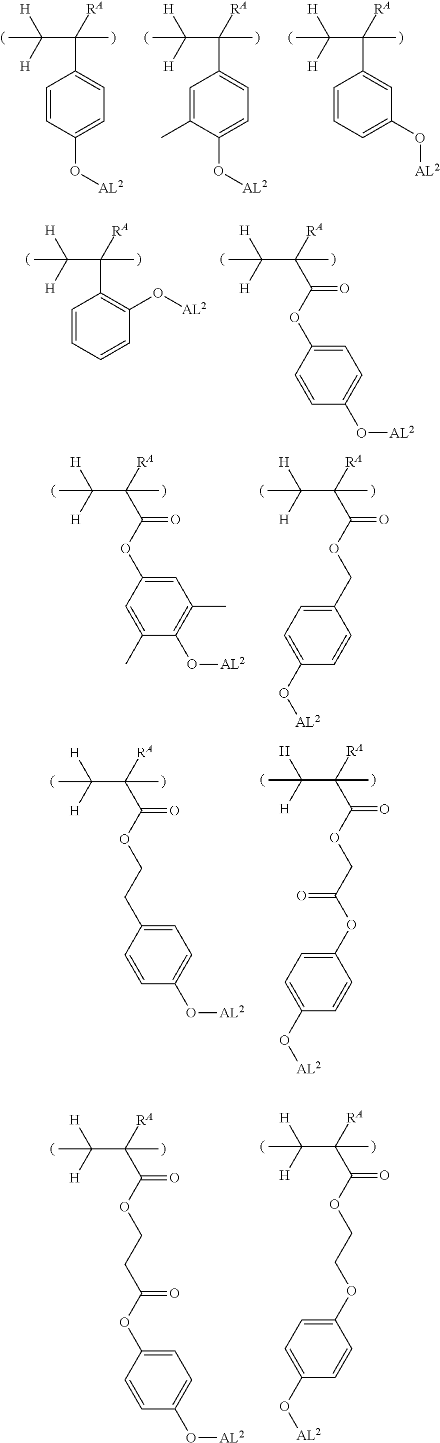

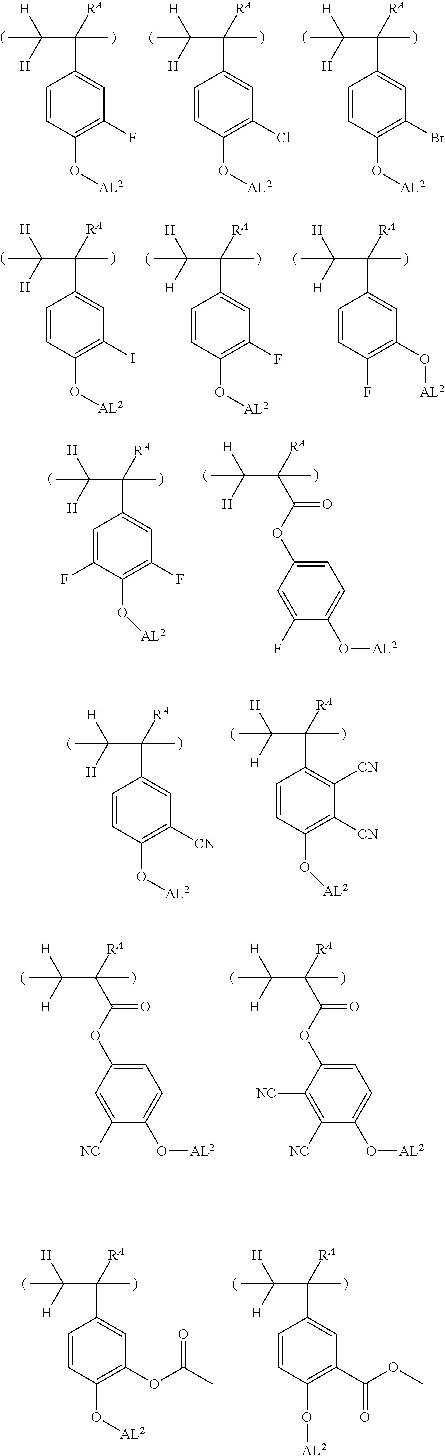

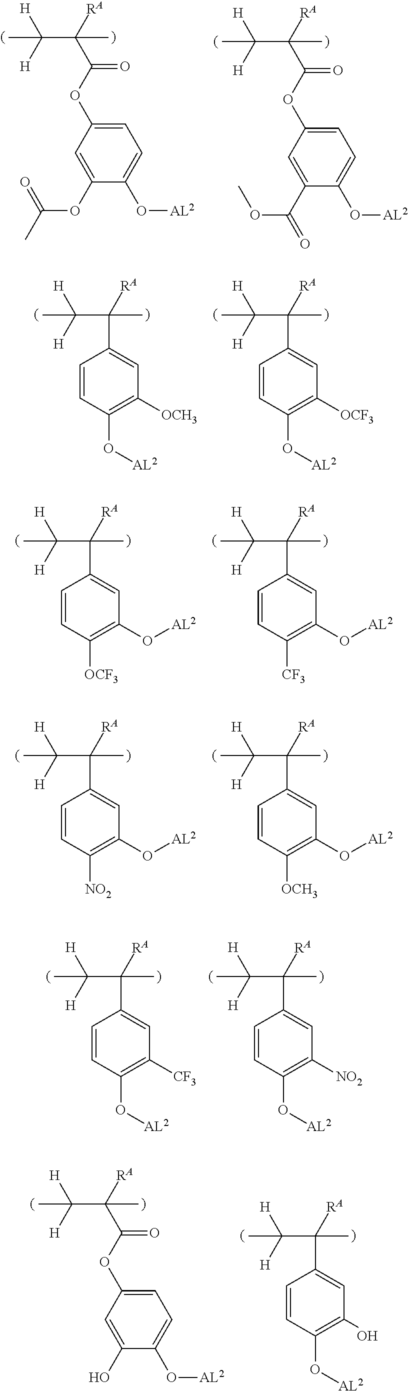

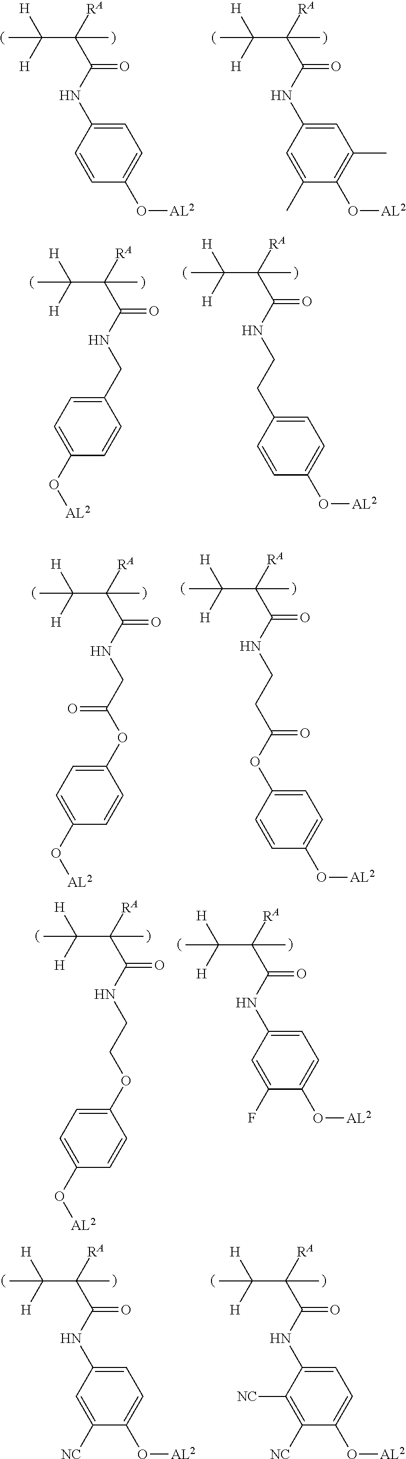

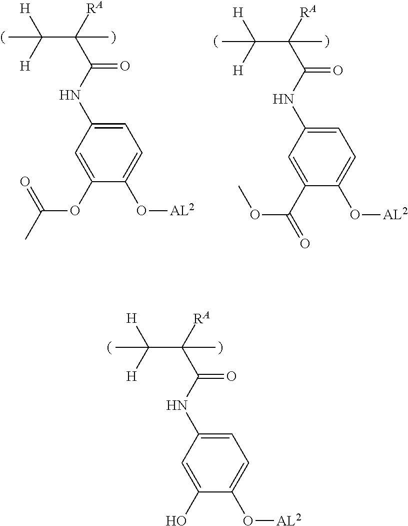

In a preferred embodiment, the polymer comprises repeat units having the formula (a3), which are simply referred to as repeat units (a3).

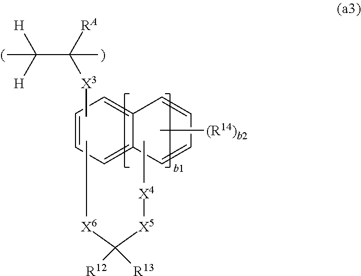

In formula (a3), b1 is 0 or 1. The relevant structure is a benzene ring in case of b1=0, and a naphthalene ring in case of b1=1. The benzene ring corresponding to b1=0 is preferred from the standpoint of solvent solubility. The subscript b2 is 0, 1, 2 or 3 in case of b1=0 and 0, 1, 2, 3, 4 or 5 in case of b1=1. It is preferred from the standpoint of availability of reactants that b2 be 0, 1, 2 or 3, more preferably 0, 1 or 2.

In formula (a3), R^{A} is hydrogen, fluorine, methyl or trifluoromethyl, preferably hydrogen or methyl, more preferably hydrogen.

In formula (a3), X³ is a single bond, *—C(═O)—O— or *—C(═O)—NH—, wherein * designates a point of attachment to the carbon atom in the backbone. X³ is preferably a single bond or *—C(═O)—O—, more preferably a single bond.

In formula (a3), X⁴ is a single bond, C₁-C₄ aliphatic hydrocarbylene group, carbonyl group, sulfonyl group or a group obtained by combining the foregoing. Inter alia, a single bond, carbonyl group or sulfonyl group is preferred from the aspect of reactant availability, and a single bond or carbonyl group is more preferred from polar groups formed after the reaction.

In formula (a3), X⁵ and X⁶ are each independently oxygen or sulfur. Notably, X⁴ and X⁶ are bonded to vicinal carbon atoms on the aromatic ring. X⁵ and X⁶ may be identical or different. It is preferred from the aspect of reactivity that X⁵ and X⁶ be both oxygen.

In formula (a3), R¹² and R¹³ are each independently hydrogen or a C₁-C₂₀ hydrocarbyl group which may contain a heteroatom. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icocyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl and naphthyl groups; C₇-C₂₀ aralkyl groups such as benzyl, 1-phenylethyl and 2-phenylethyl groups; and combinations thereof. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

Also, R¹² and R¹³ may bond together to form a ring with the carbon atom to which they are attached. Specific examples of the ring formed herein include cyclopropane, cyclobutane, cyclopentane, cyclohexane, norbornane and adamantane rings. In the ring, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

In formula (a3), R¹⁴ is halogen, hydroxy group, cyano group, nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} and R^{14B} are each independently hydrogen or a C₁-C₆ hydrocarbyl group. The halogen is preferably fluorine, chlorine, bromine or iodine, more preferably fluorine or iodine. The hydrocarbyl group and hydrocarbyl moiety of the hydrocarbyloxy, hydrocarbyloxycarbonyl and hydrocarbylthio groups may be saturated or unsaturated and straight, branched or cyclic. Examples thereof are as exemplified above for the hydrocarbyl groups R¹² and R¹³. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. When b2 is 2 or more, a plurality of R¹⁴ may be identical or different.

When b2 is 2 or more, a plurality of R¹⁴ may bond together to form a ring with the carbon atoms on the aromatic ring to which they are attached. Specific examples of the ring formed herein include cyclopropane, cyclobutane, cyclopentane, cyclohexane, norbornane and adamantane rings. In the ring, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

Examples of the repeat unit (a3) are shown below, but not limited thereto. Herein R^{A} is as defined above, and Me is methyl. The bond positions of substituents on the aromatic ring are interchangeable.

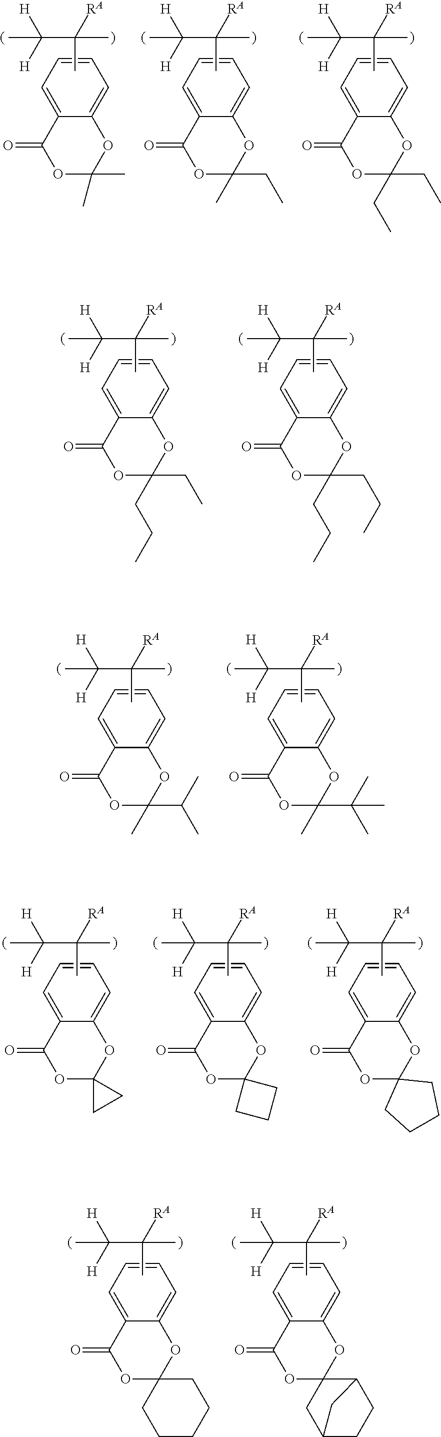

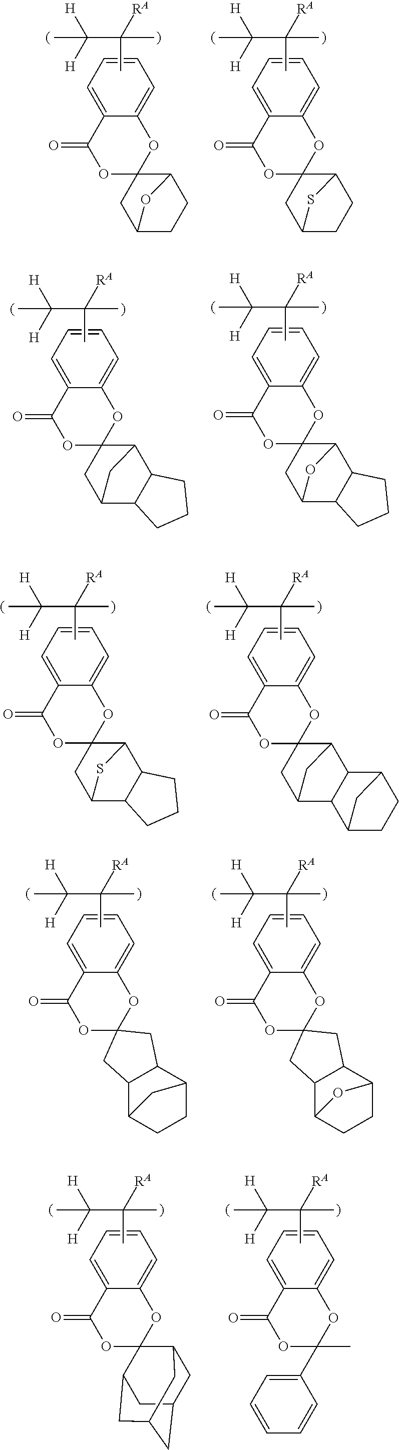

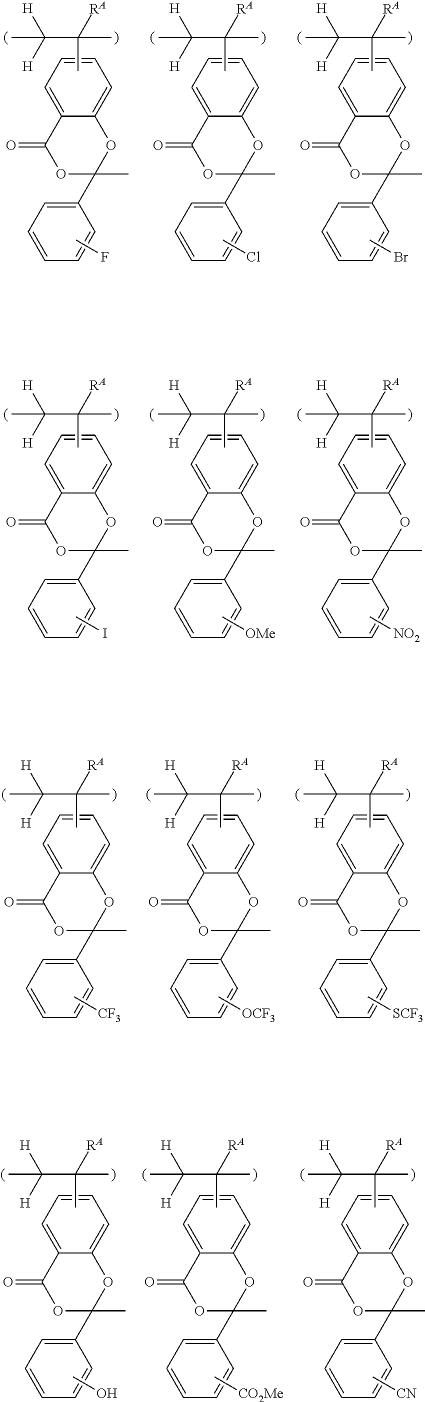

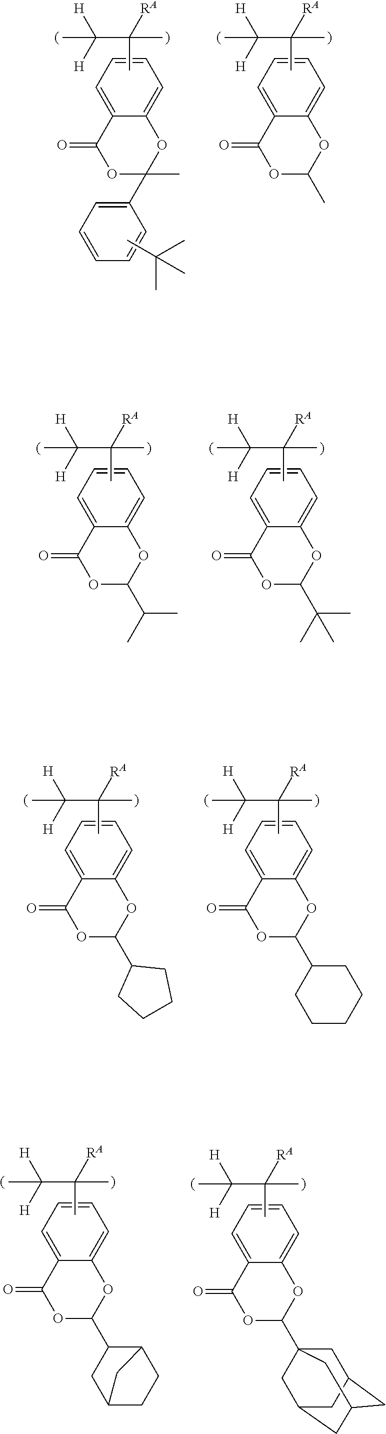

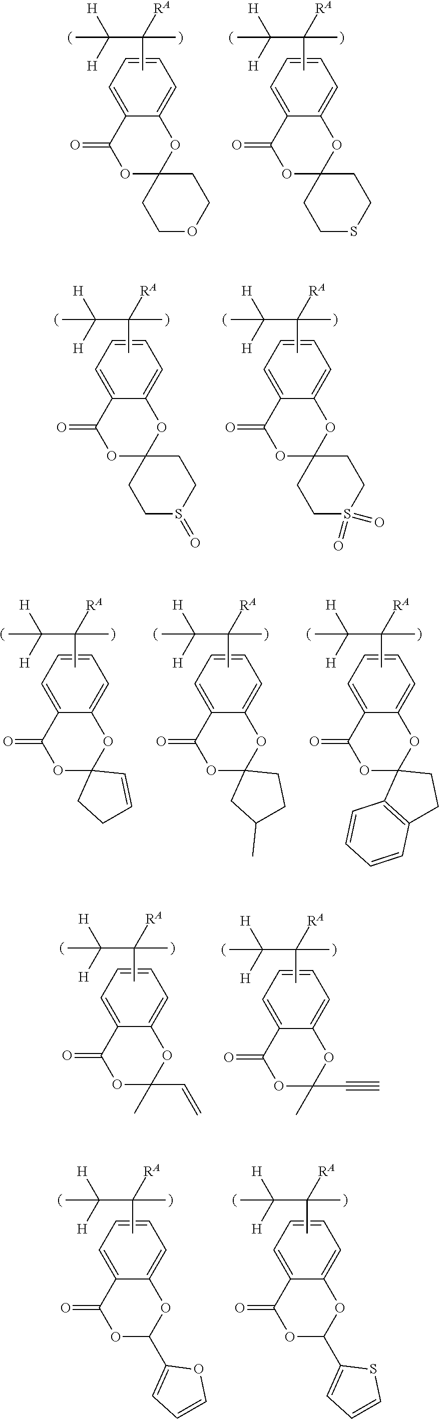

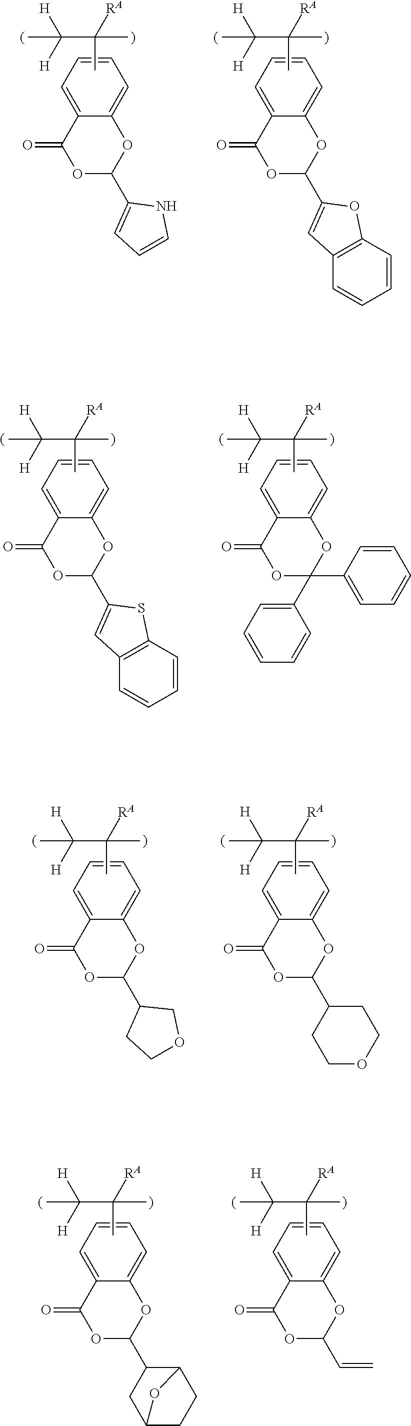

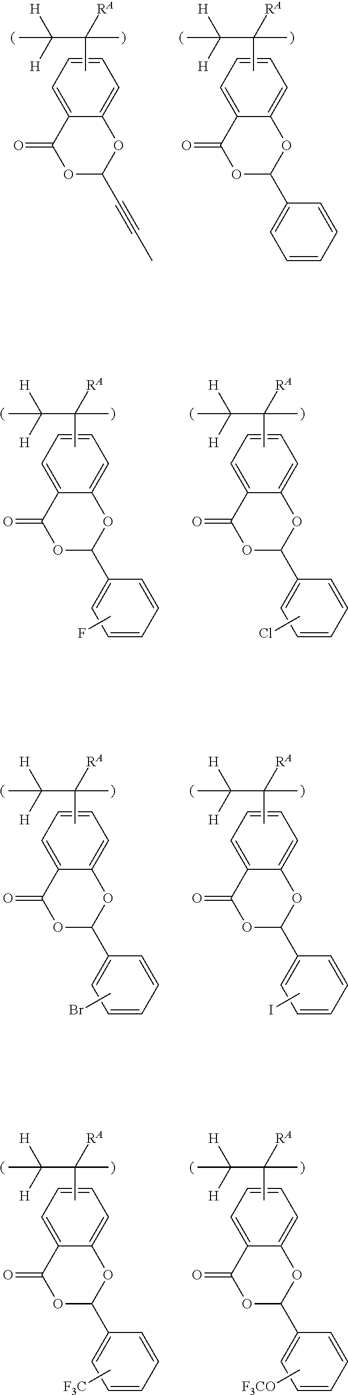

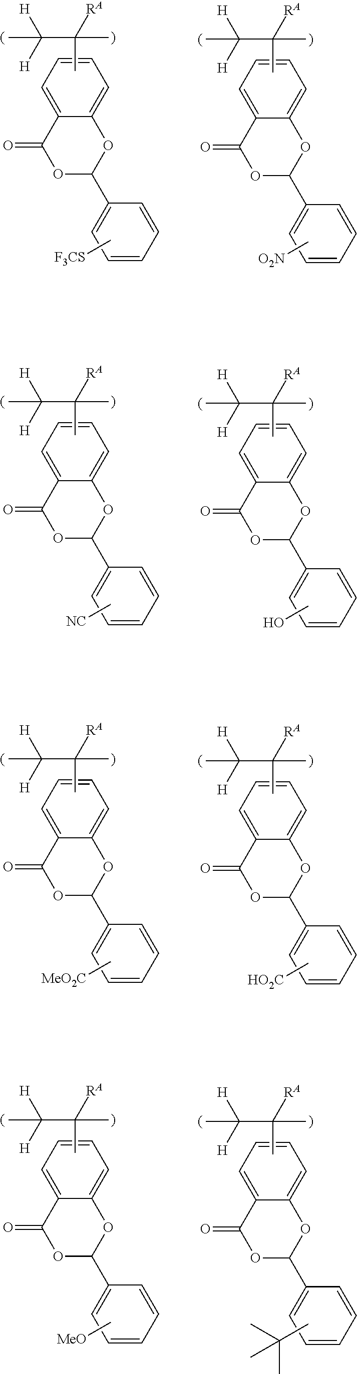

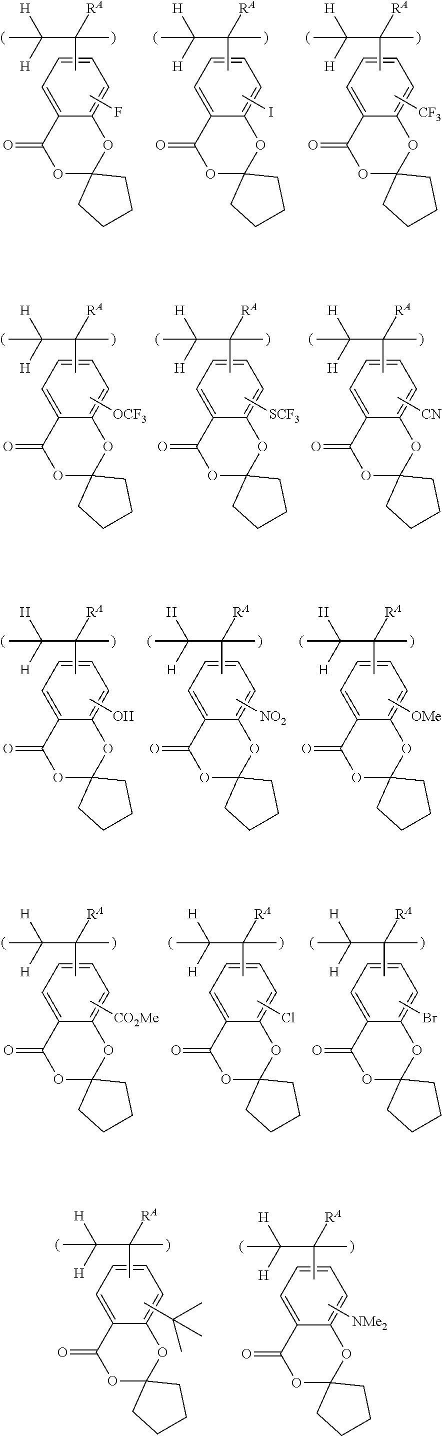

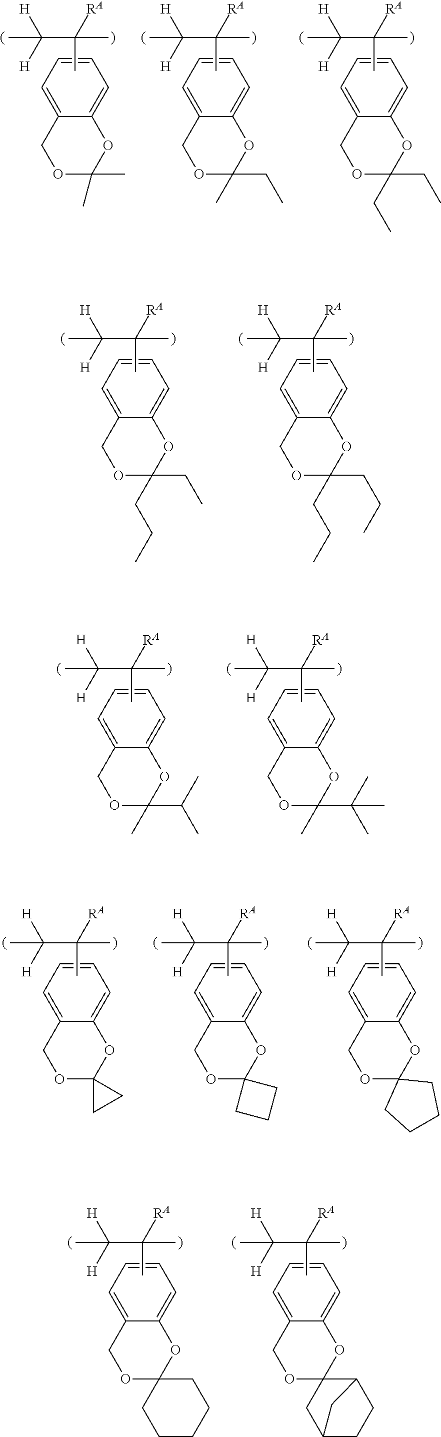

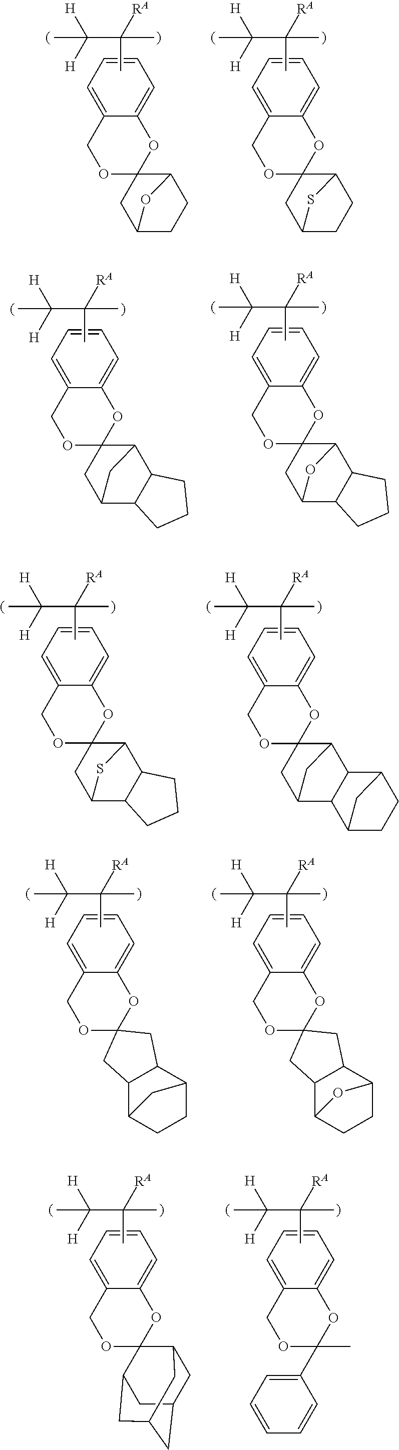

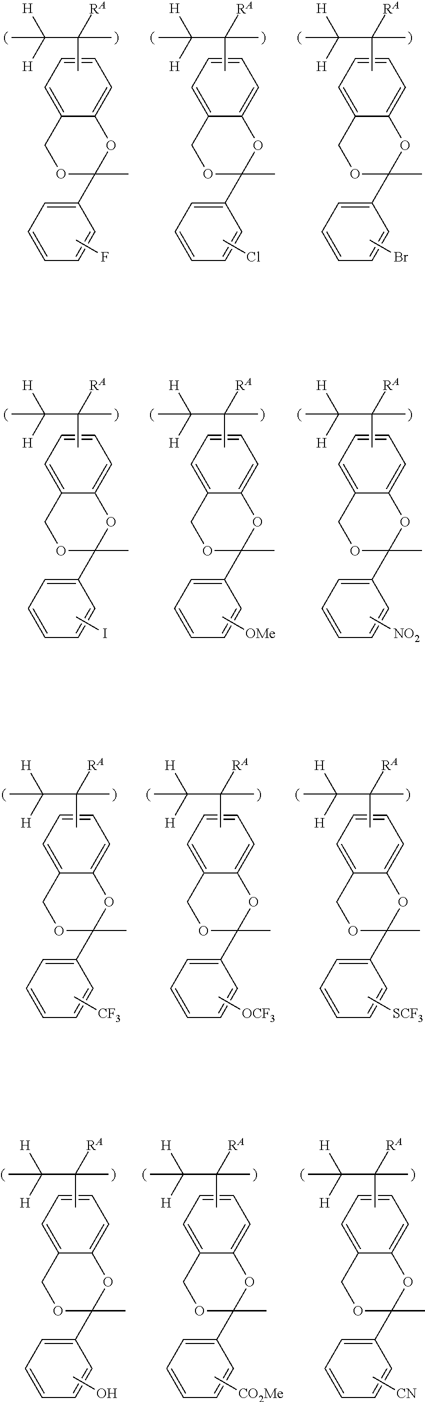

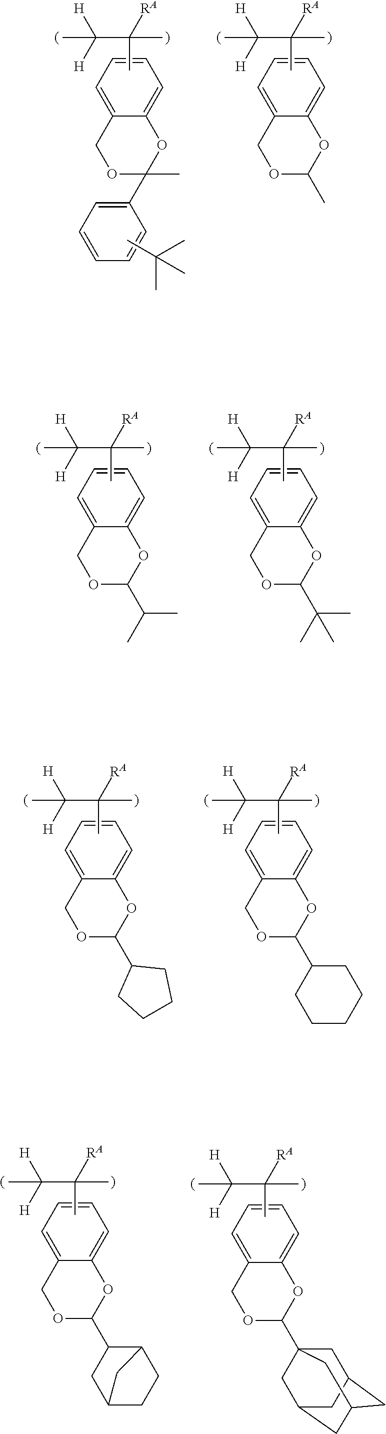

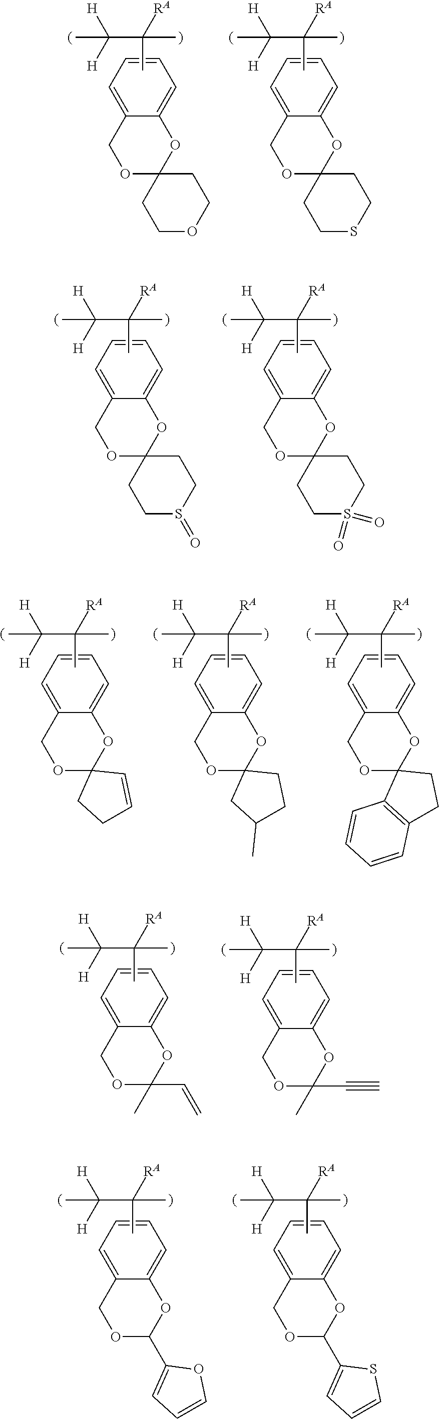

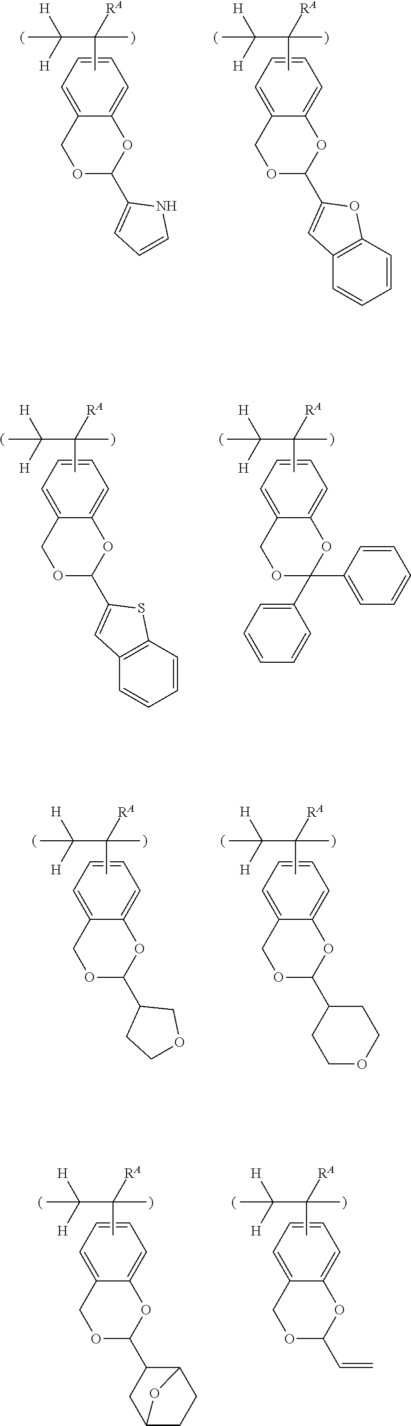

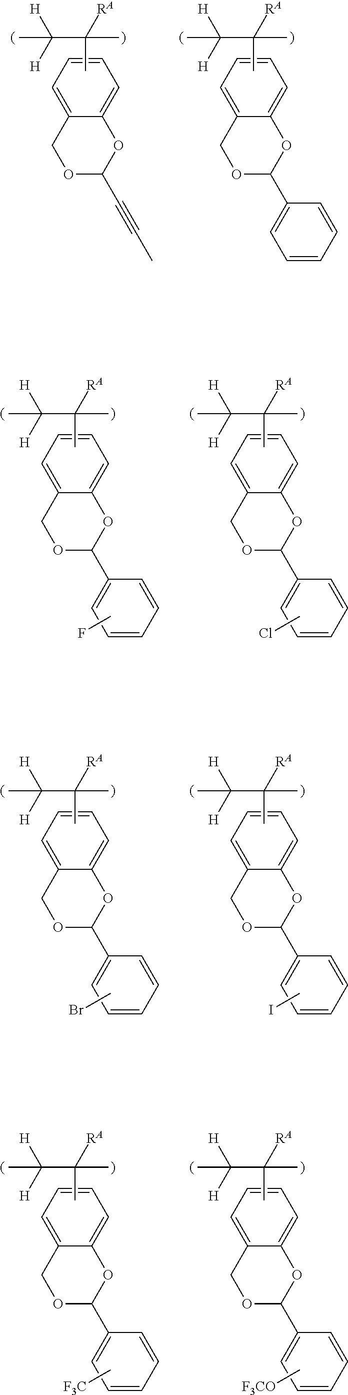

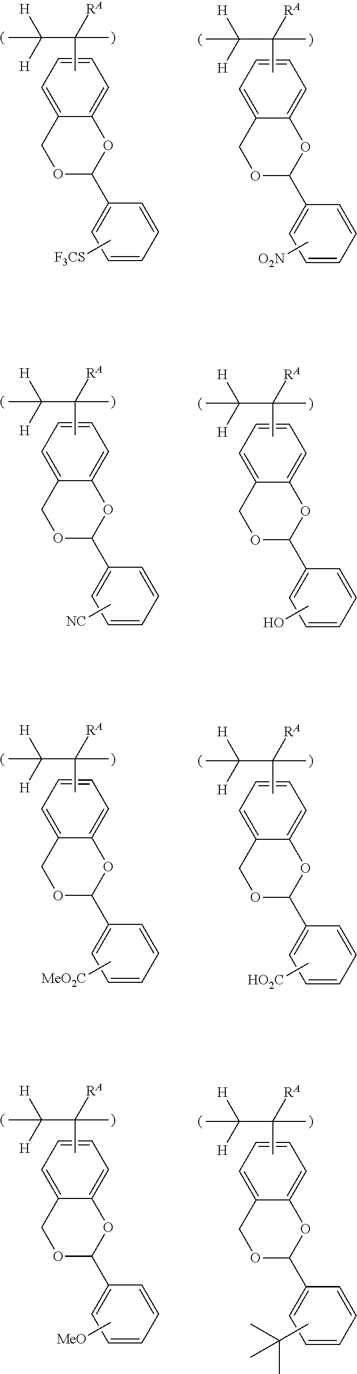

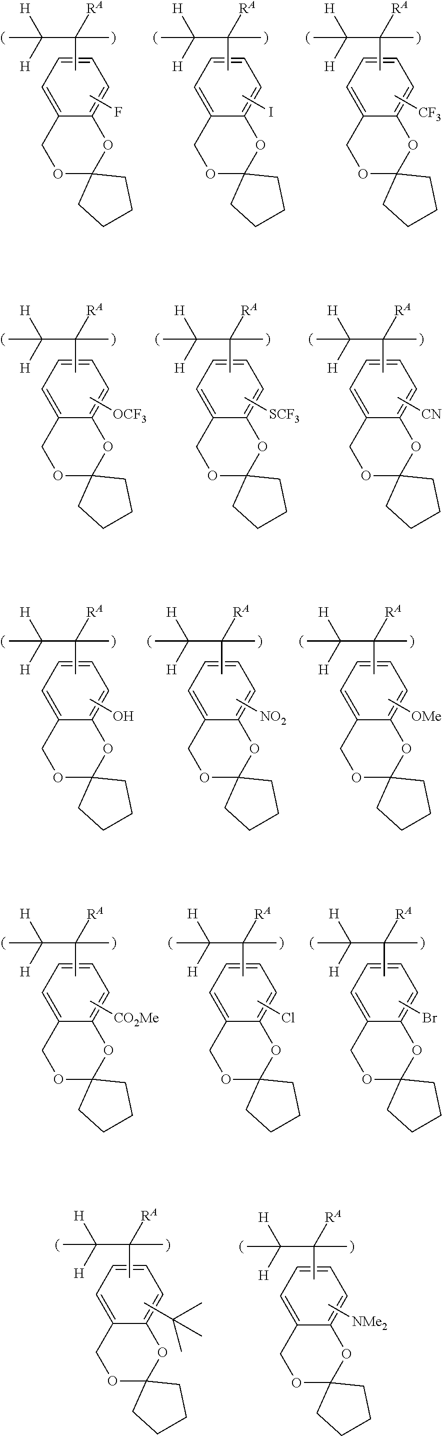

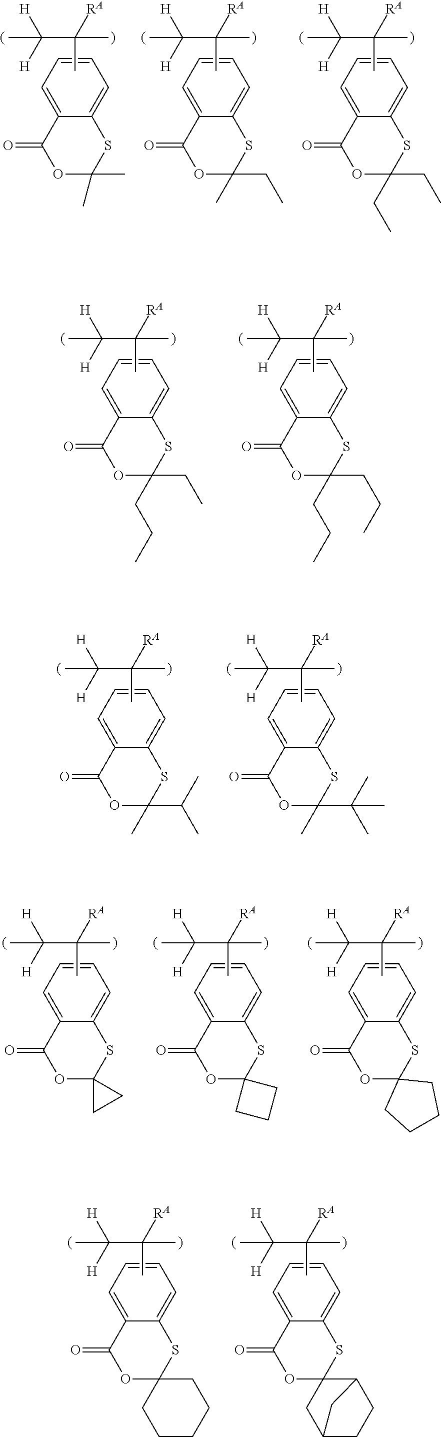

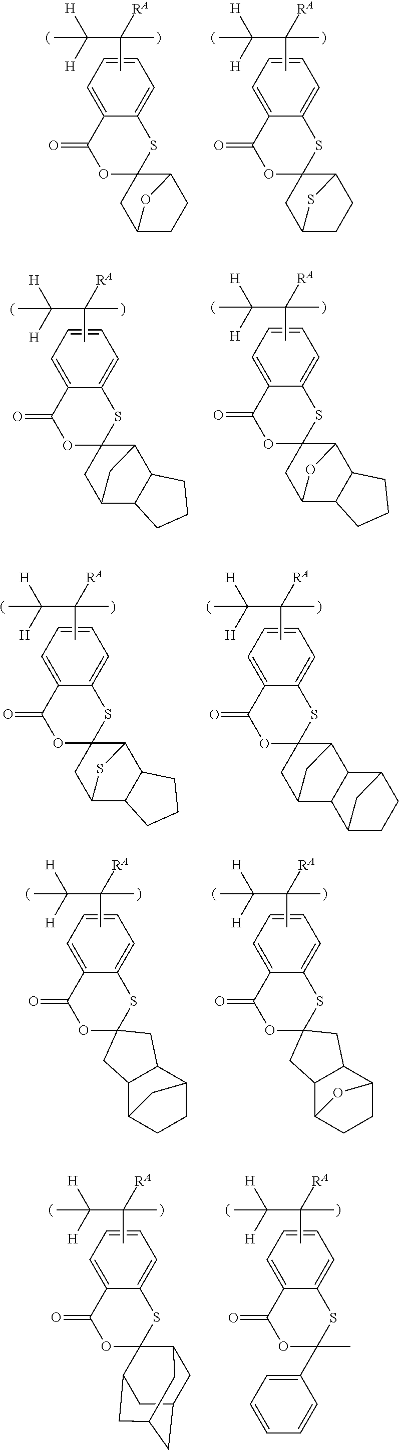

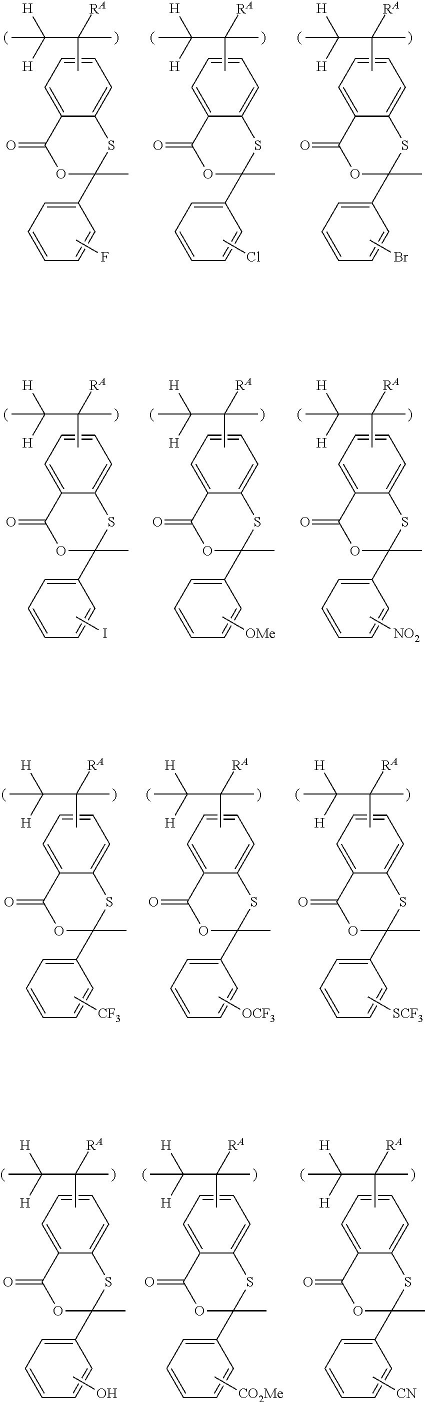

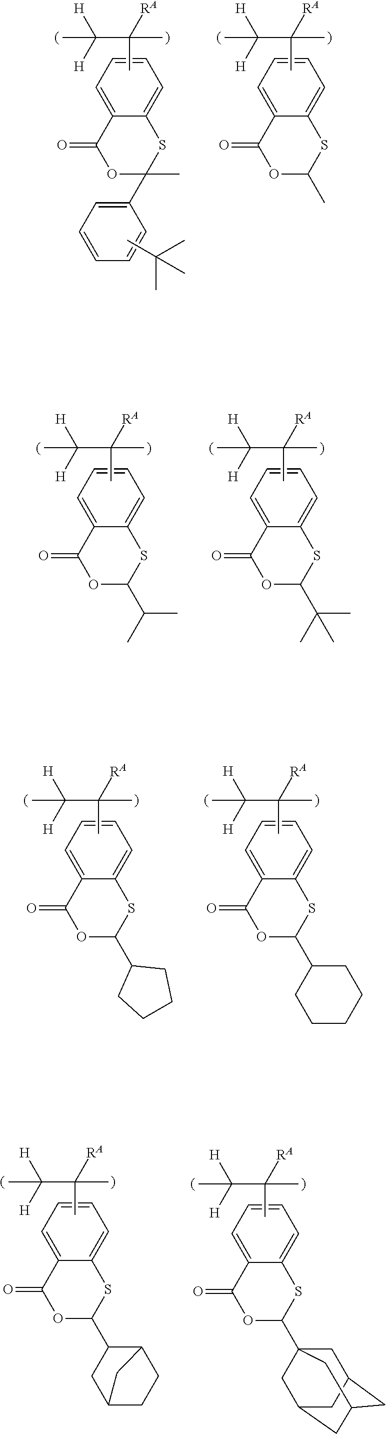

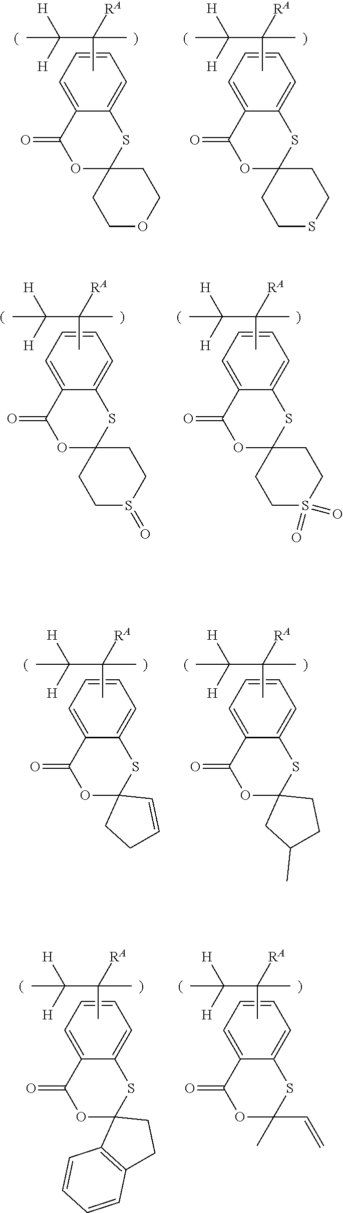

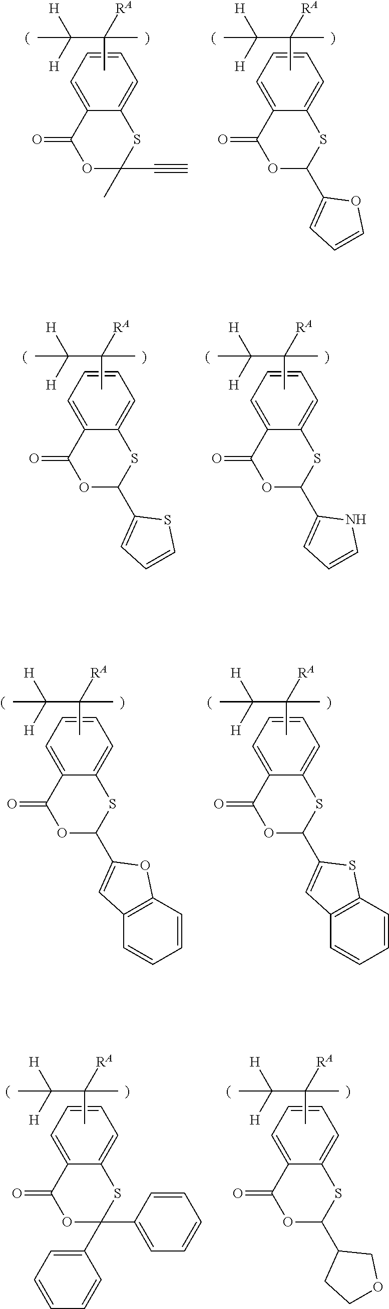

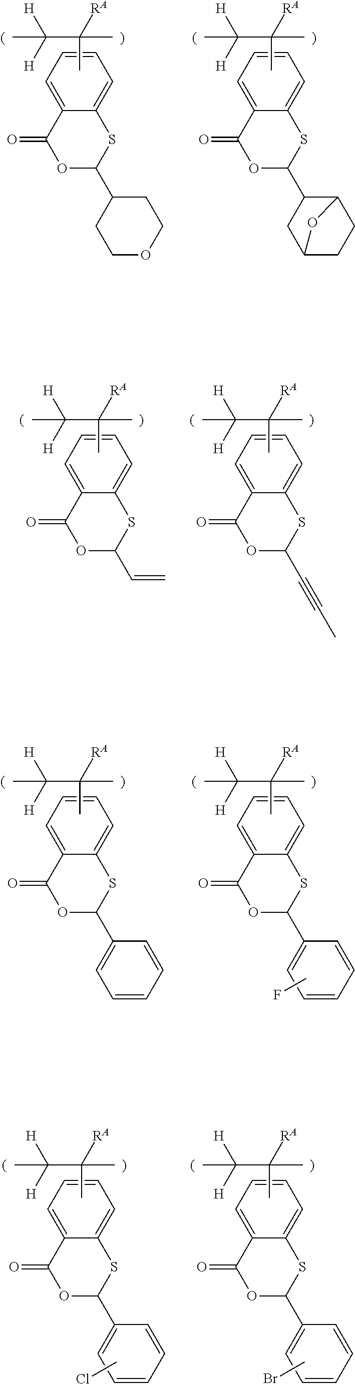

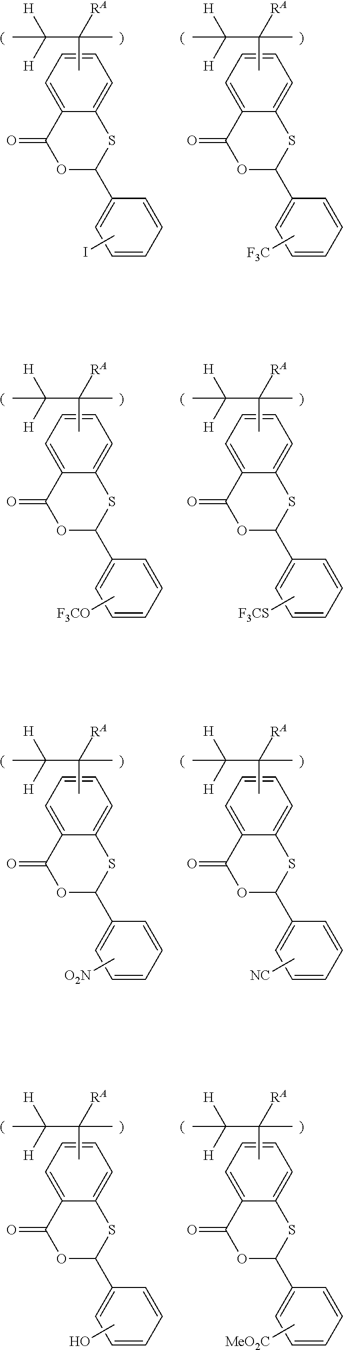

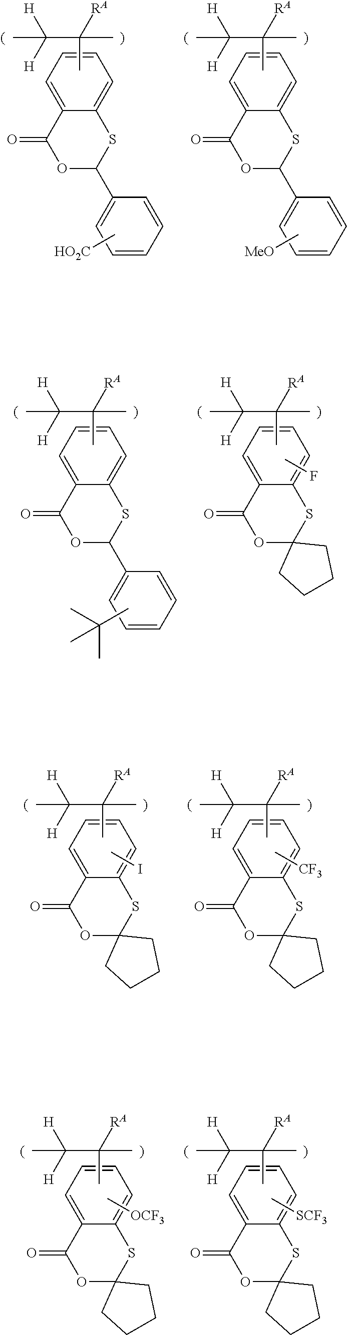

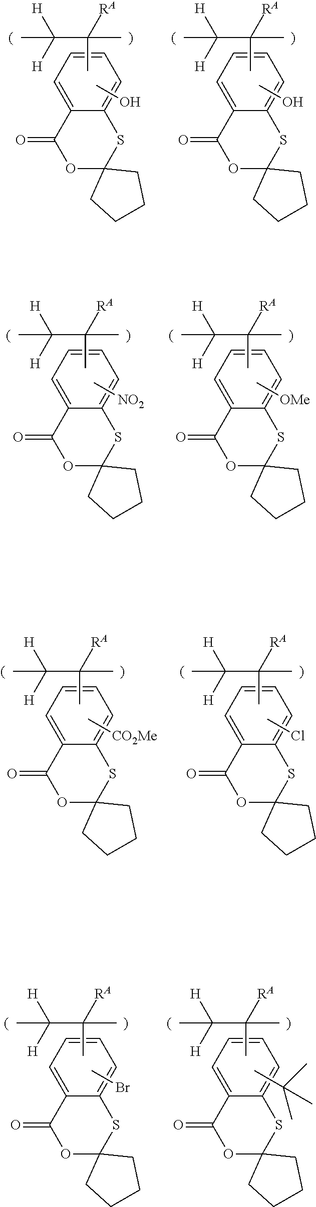

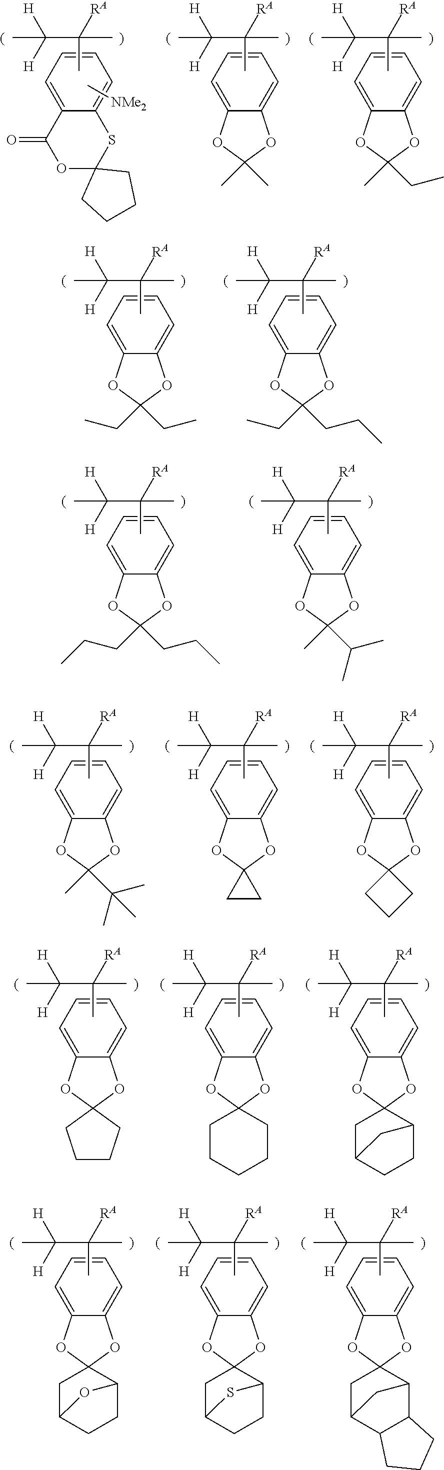

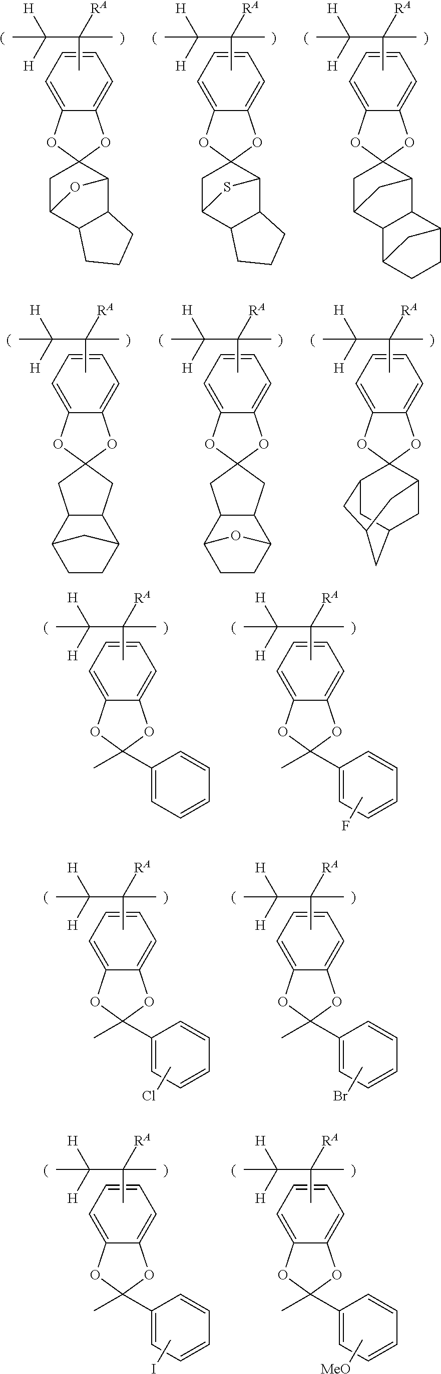

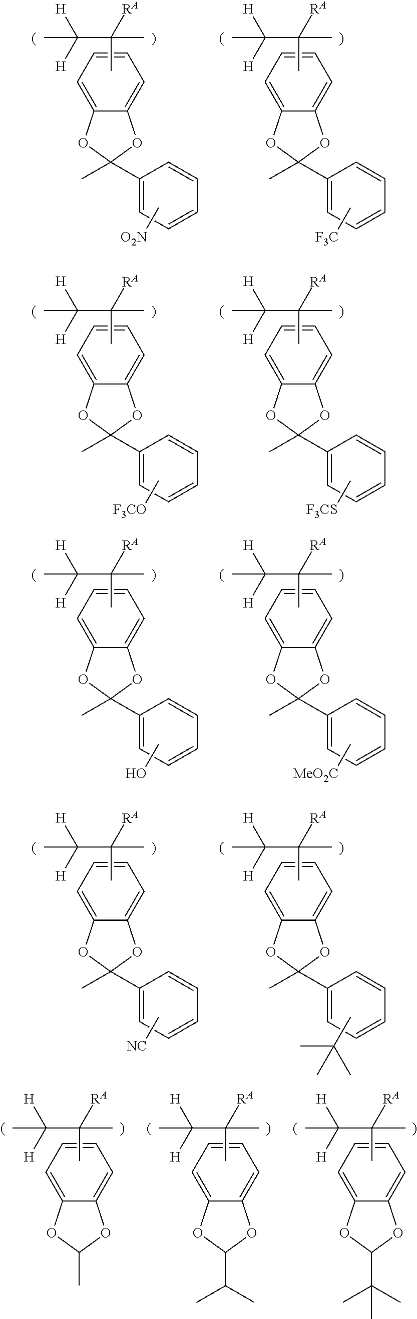

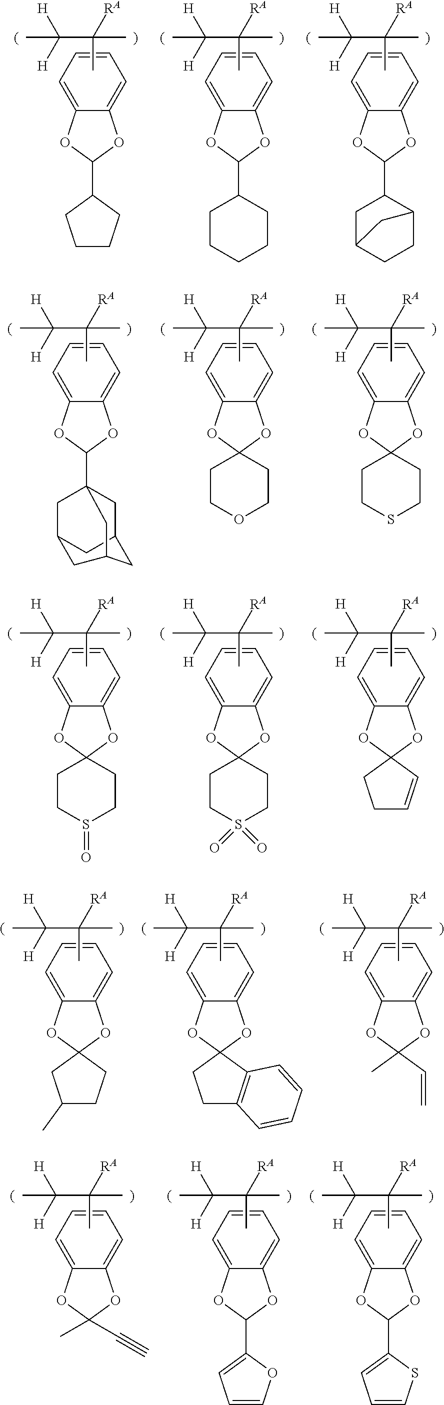

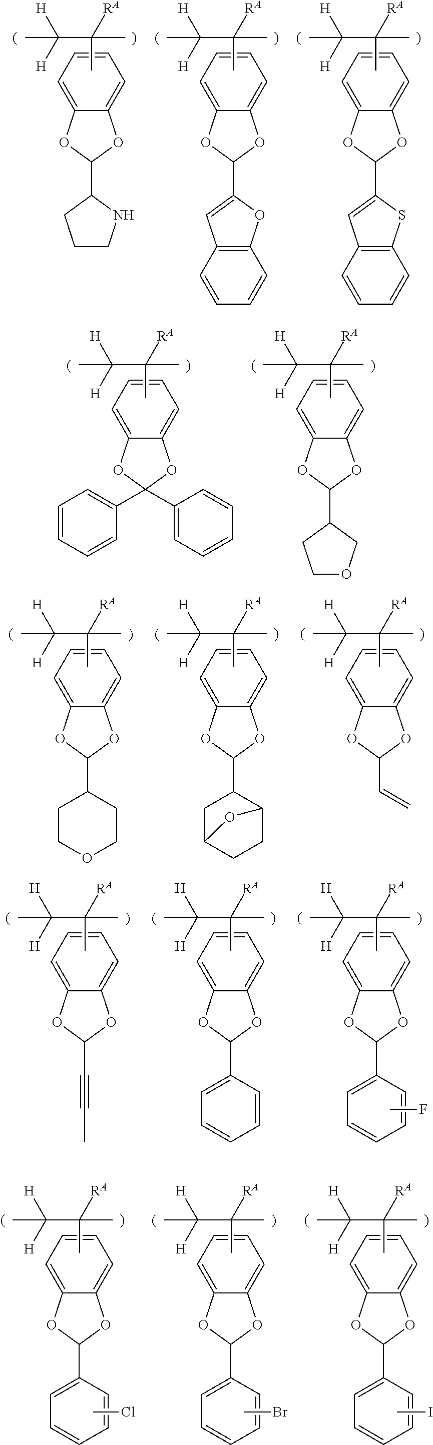

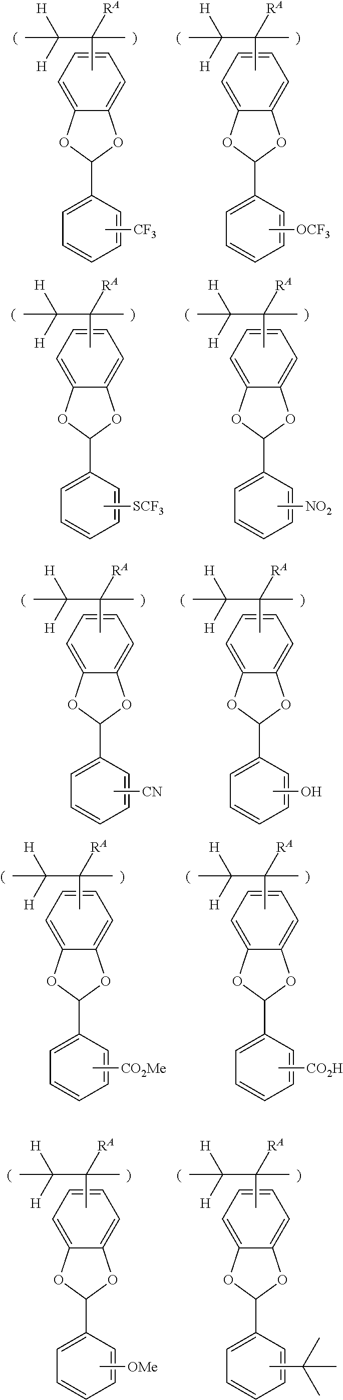

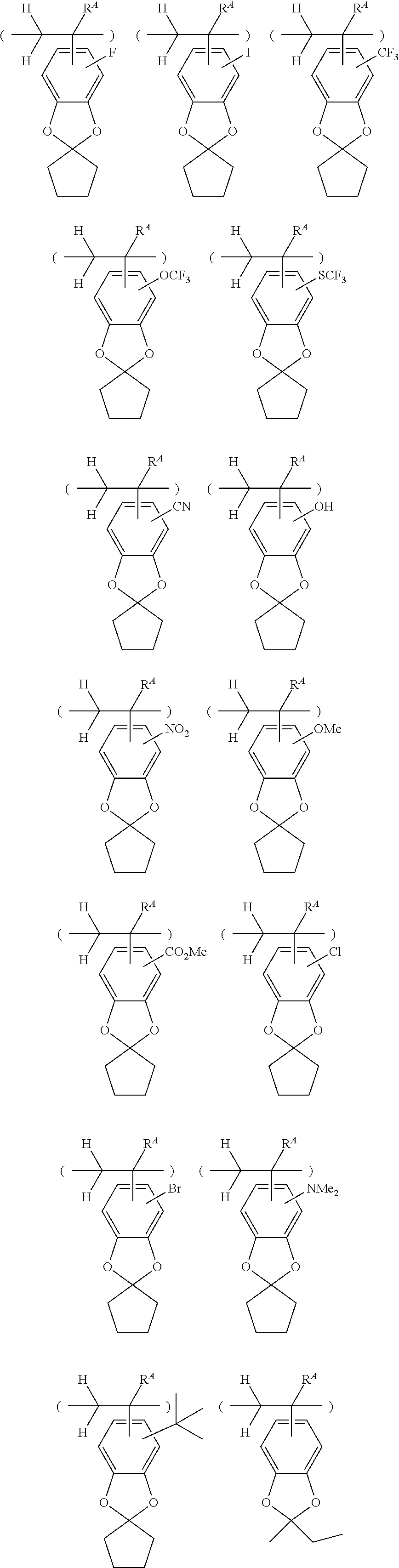

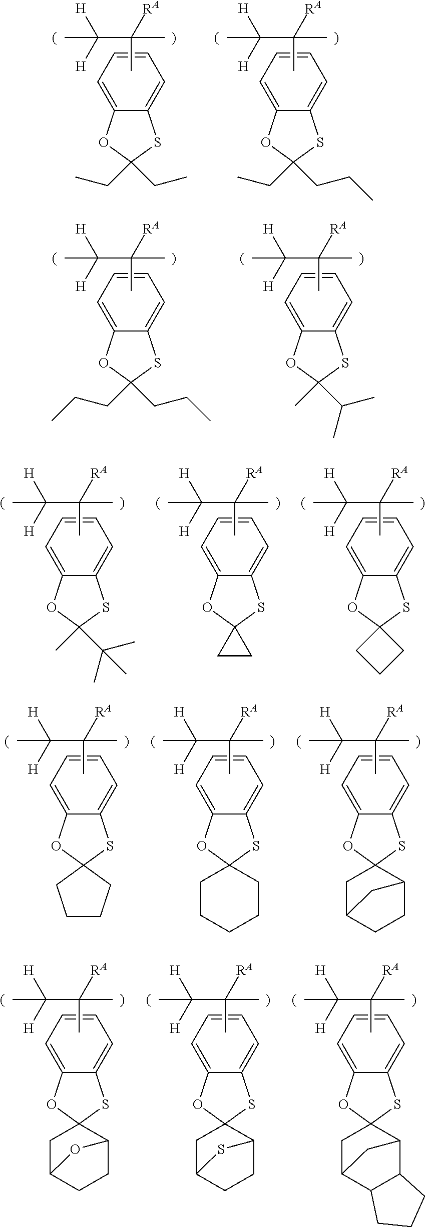

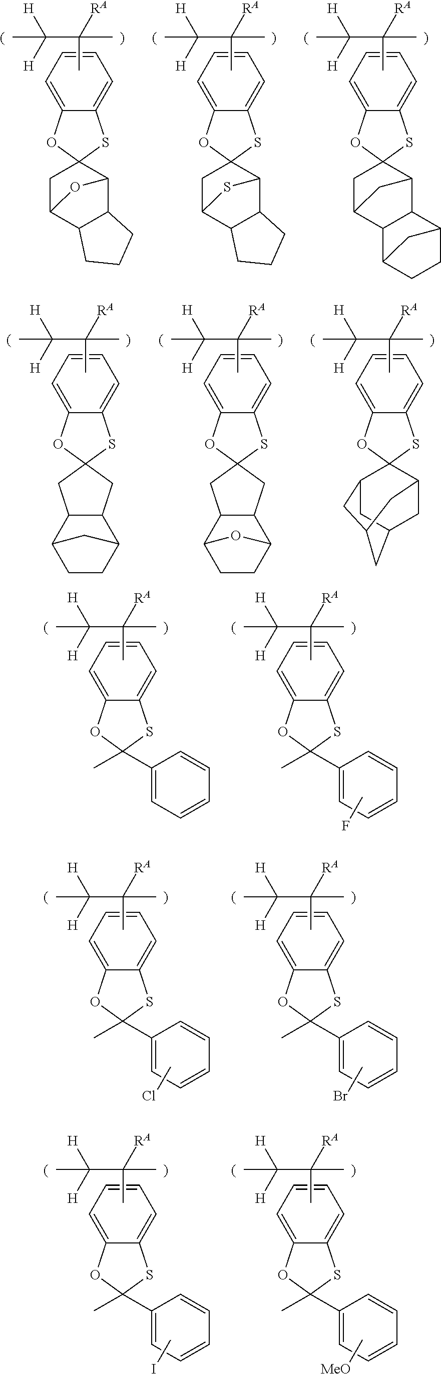

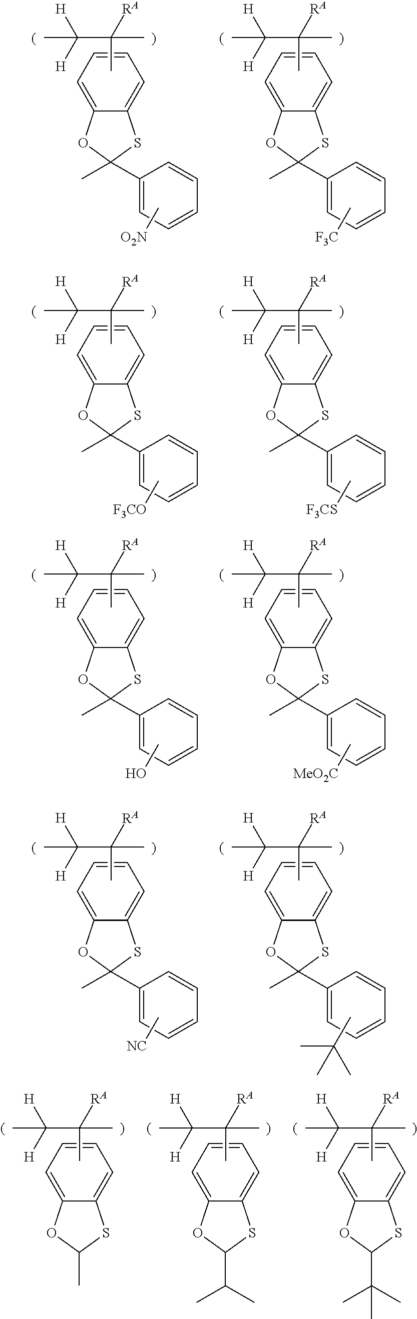

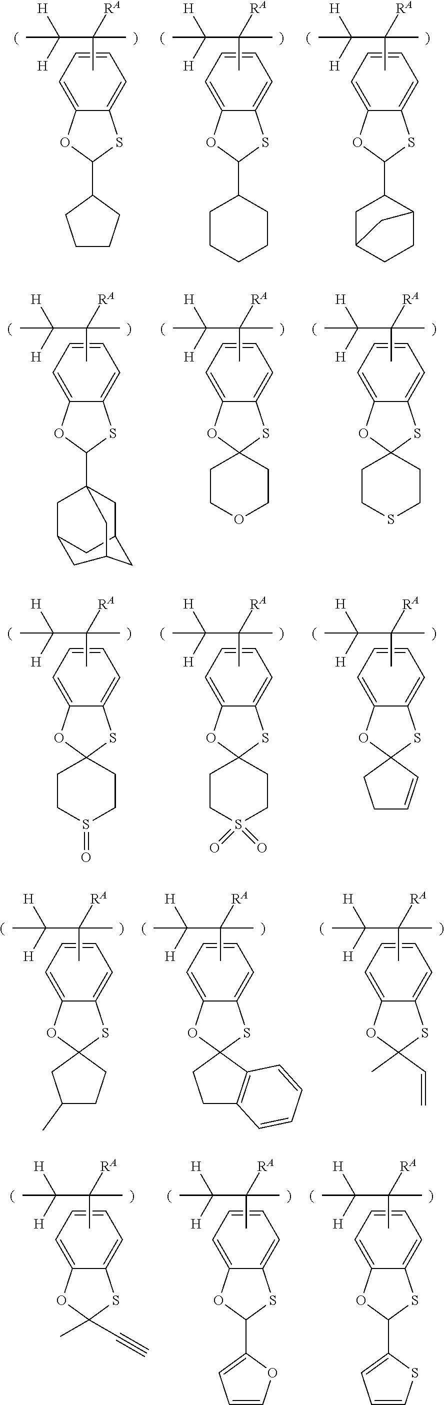

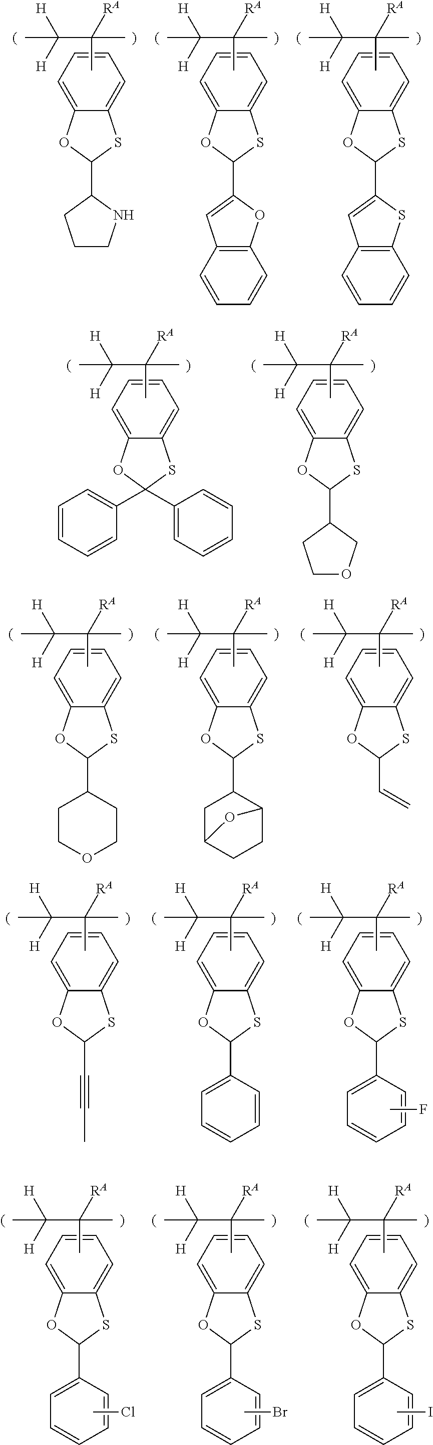

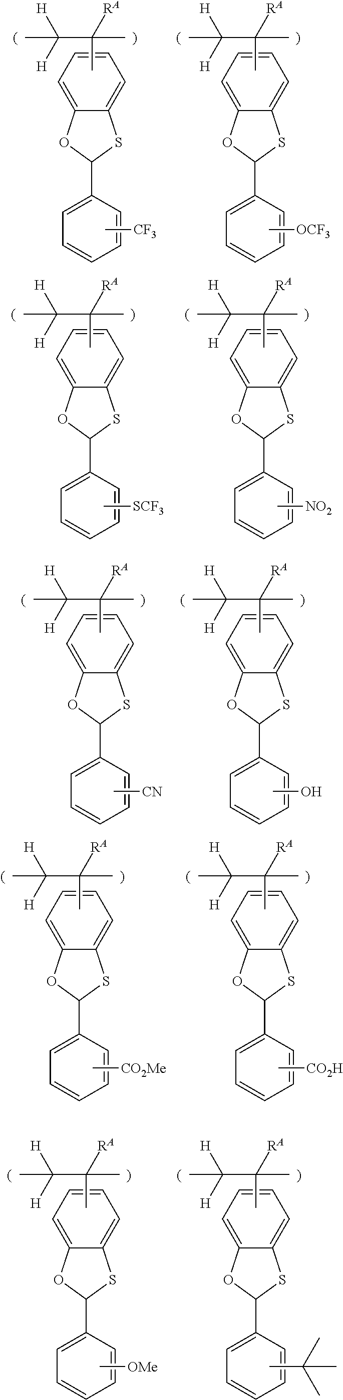

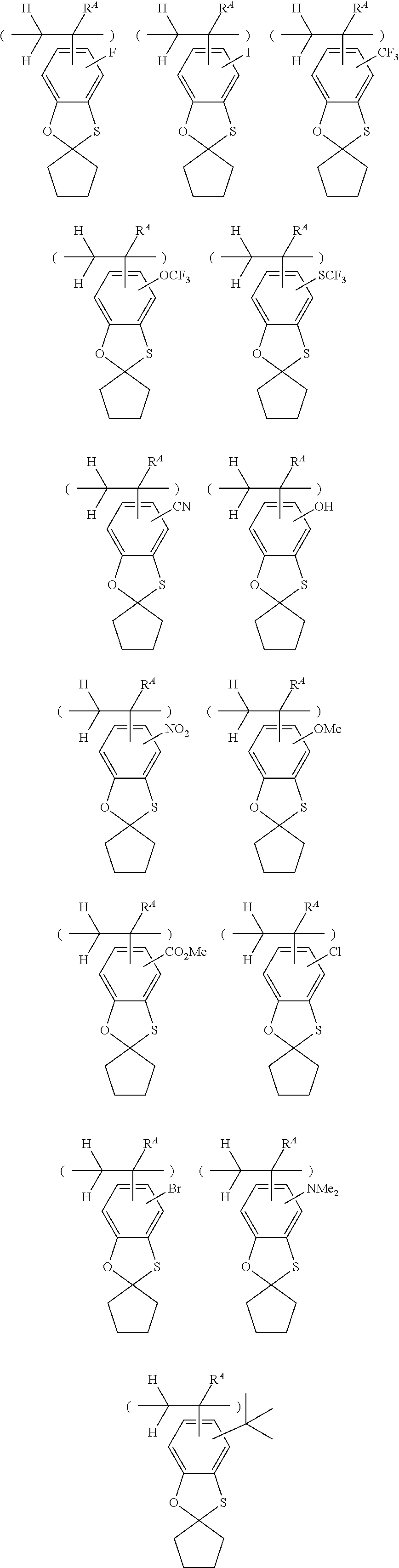

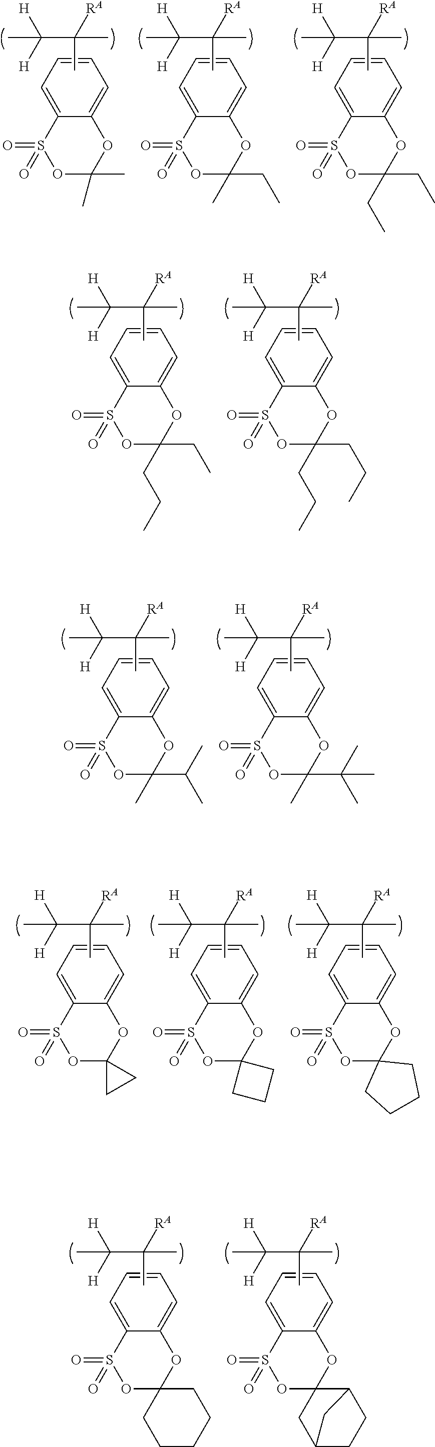

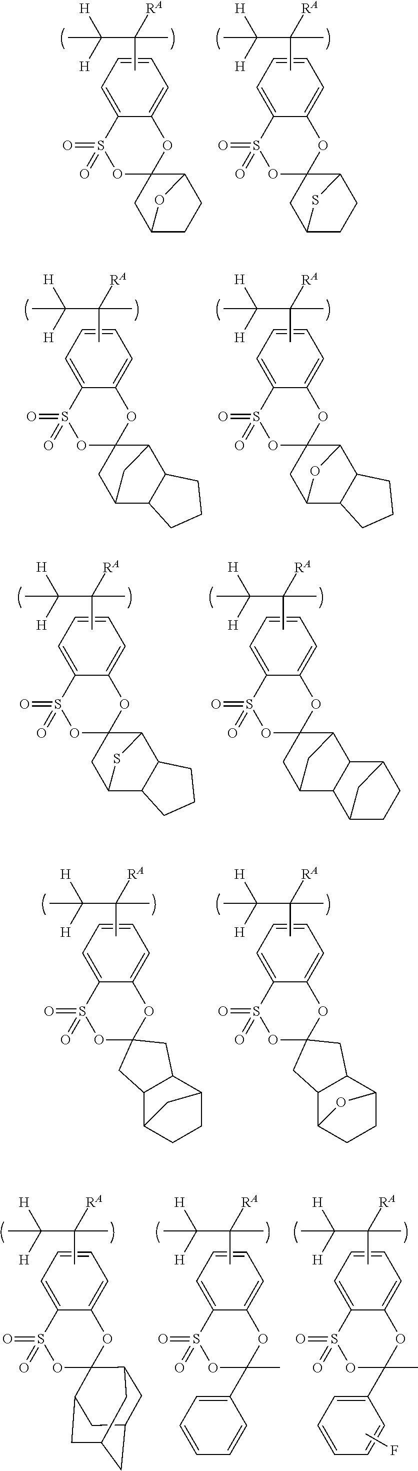

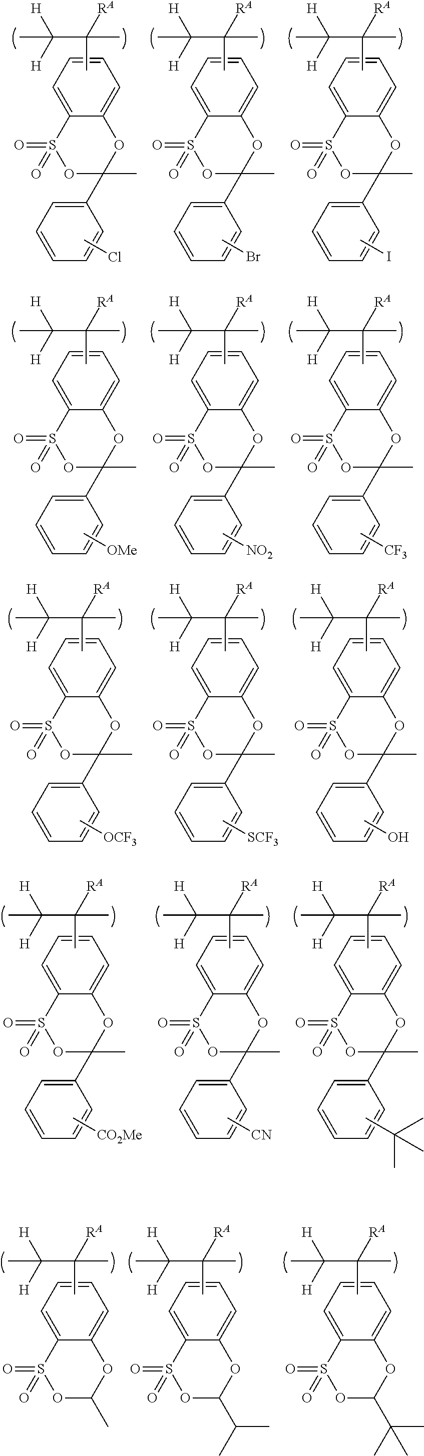

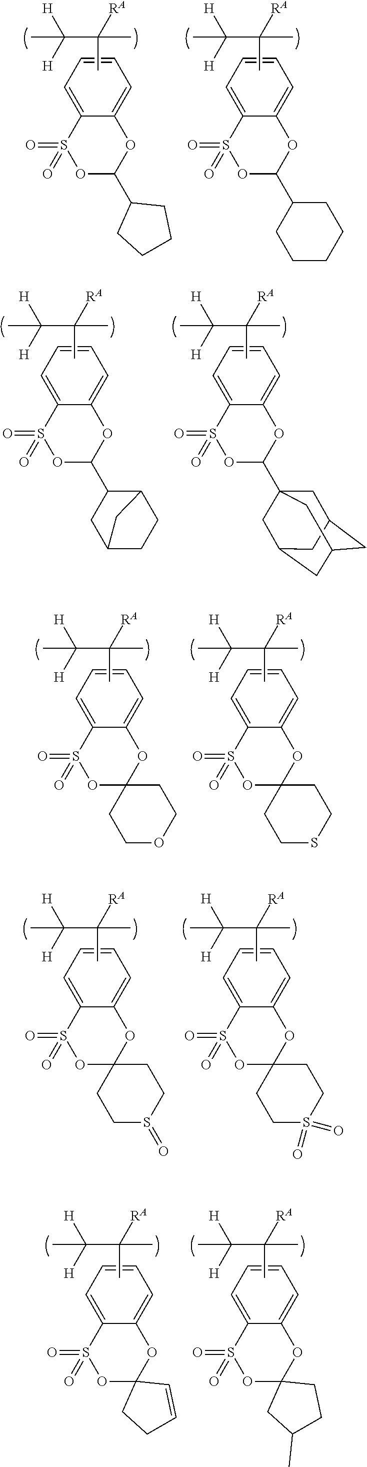

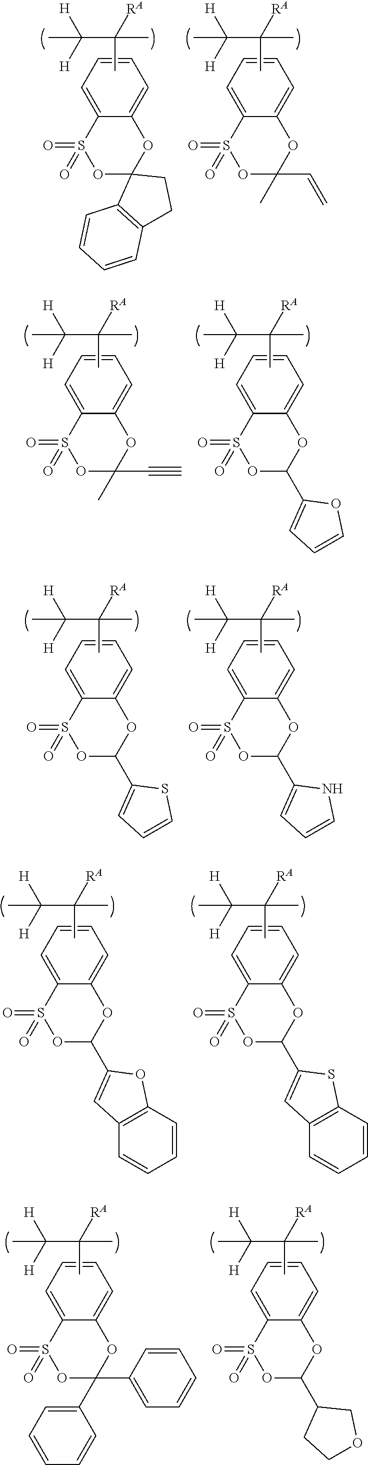

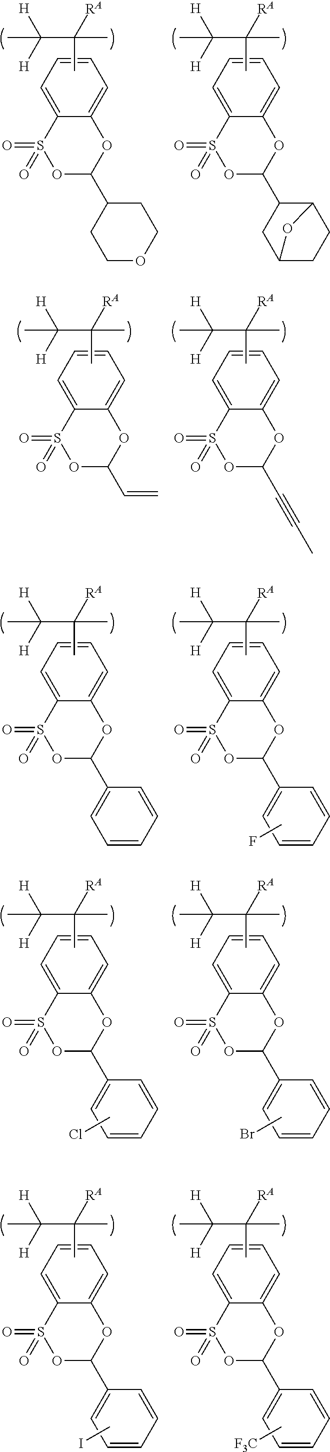

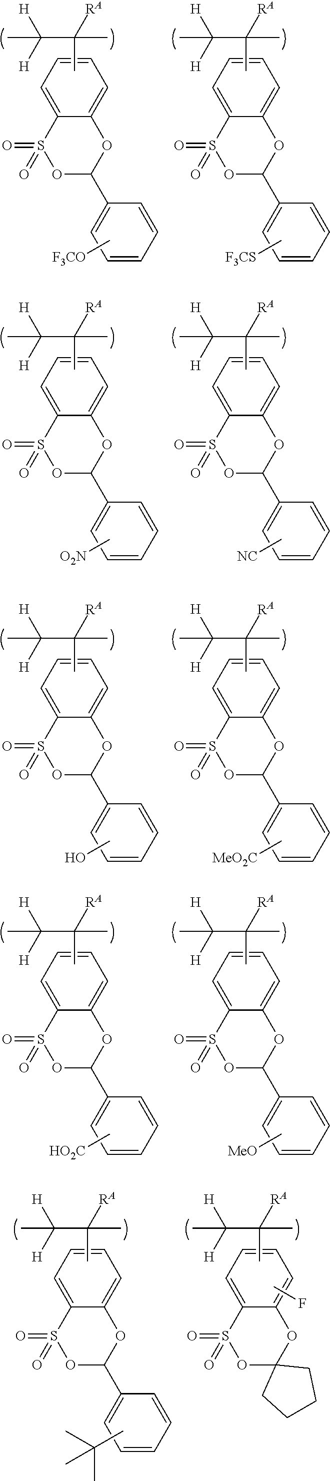

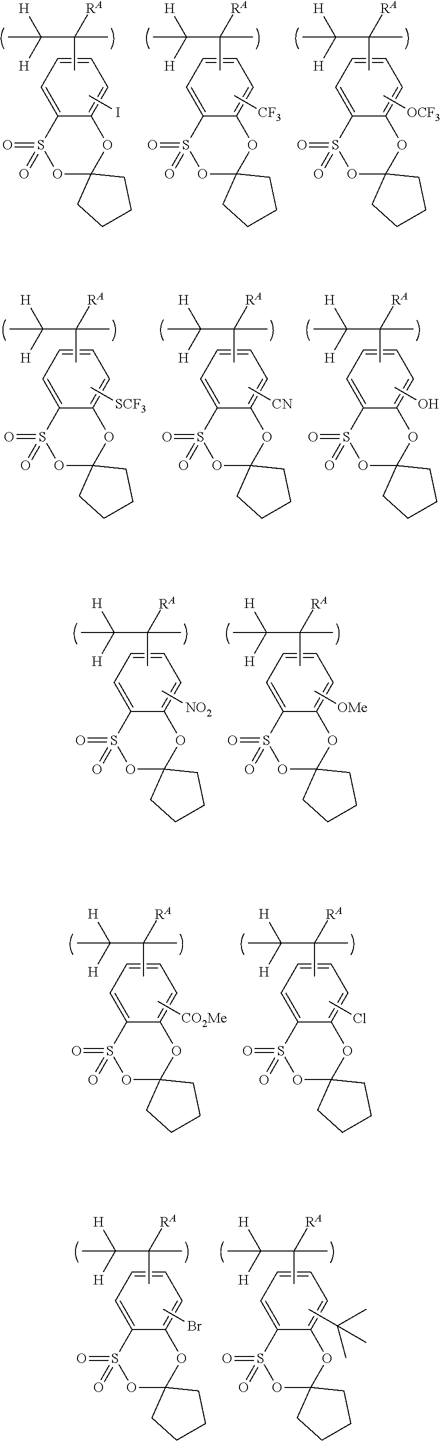

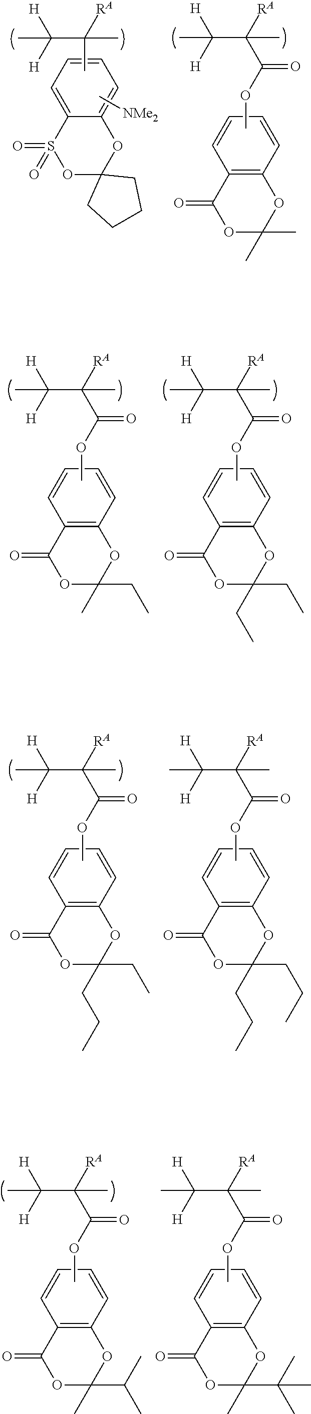

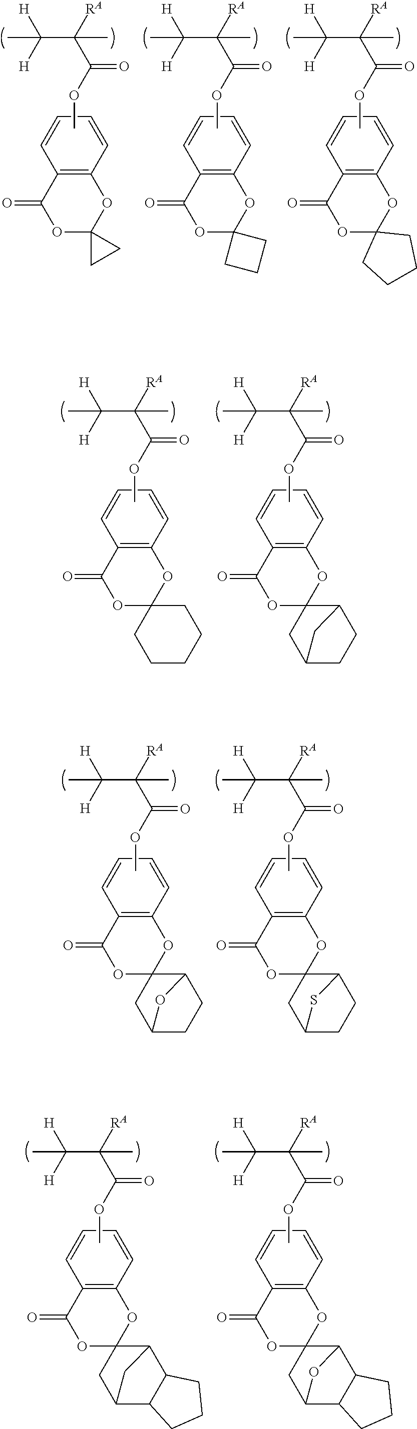

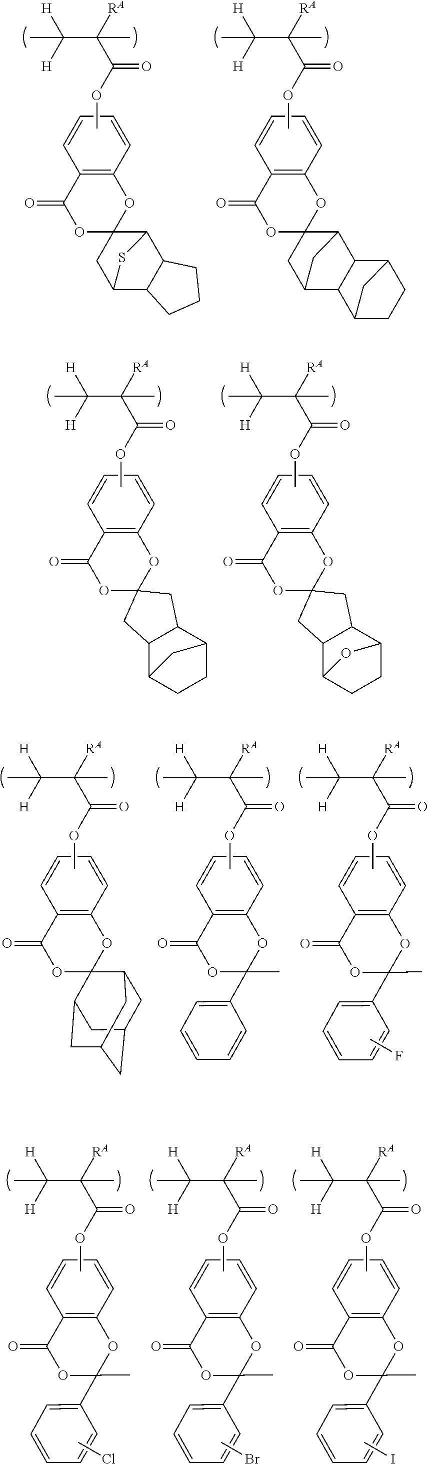

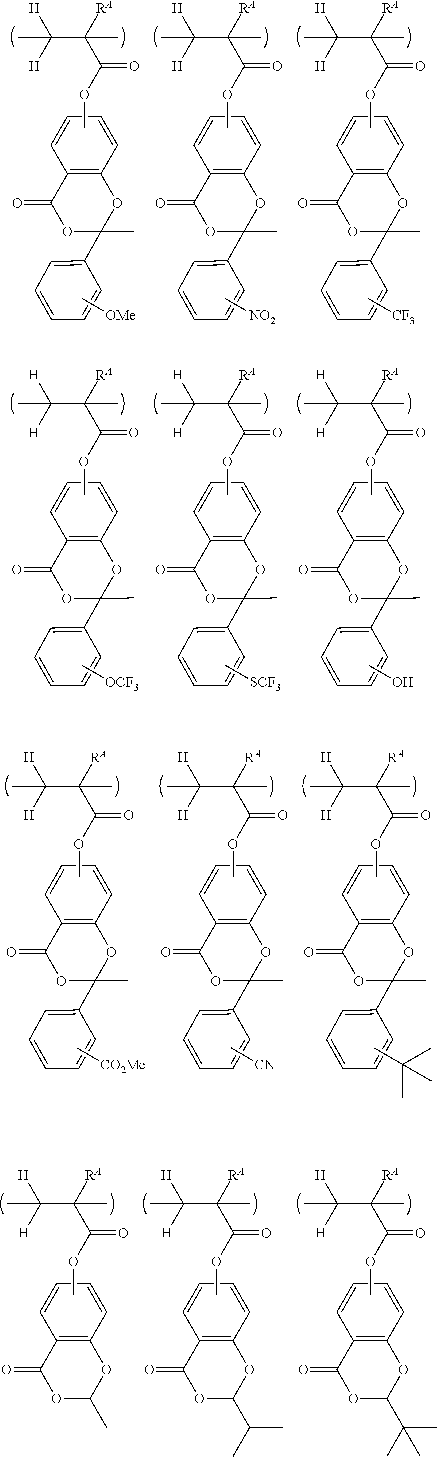

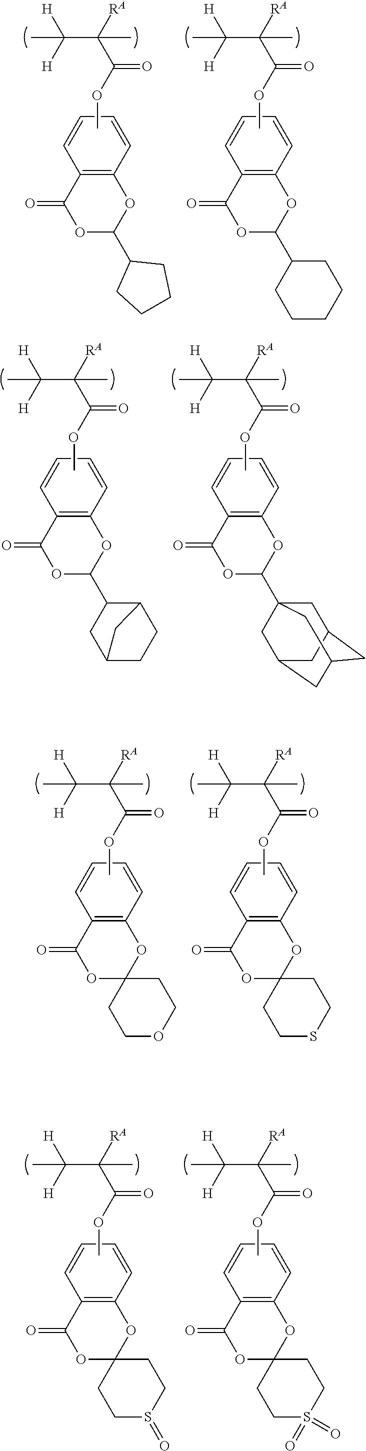

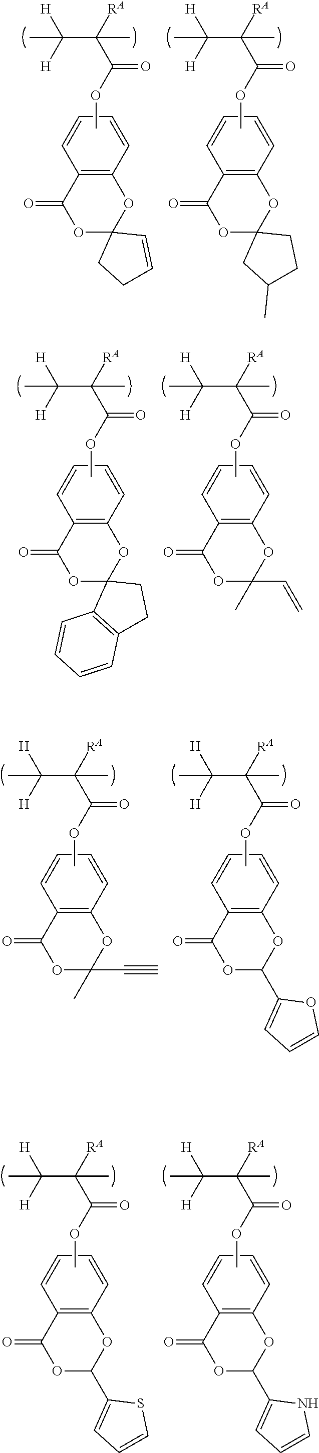

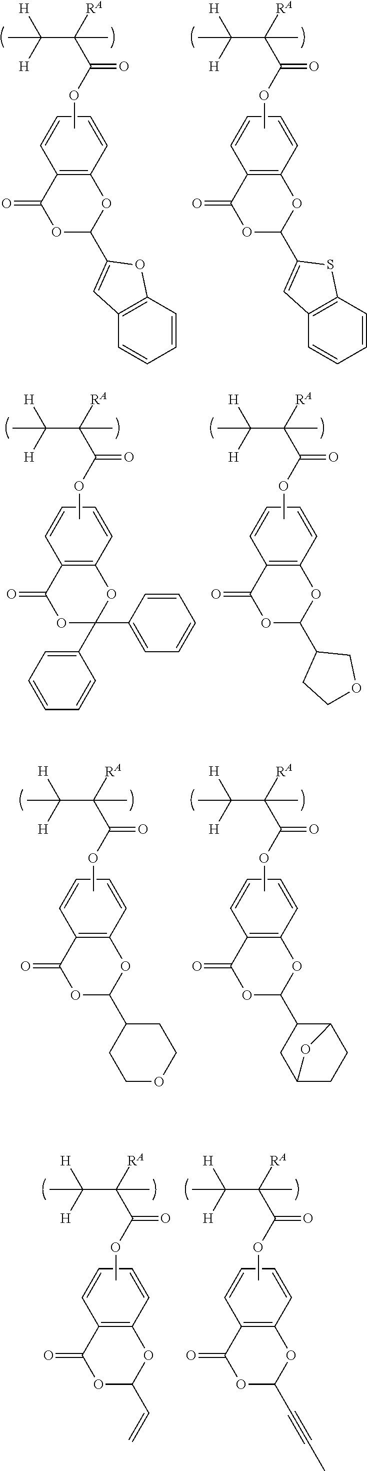

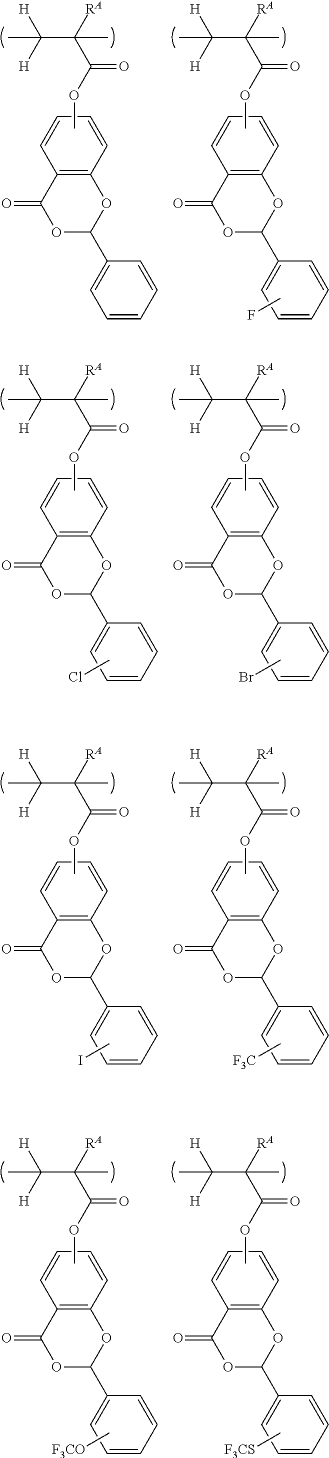

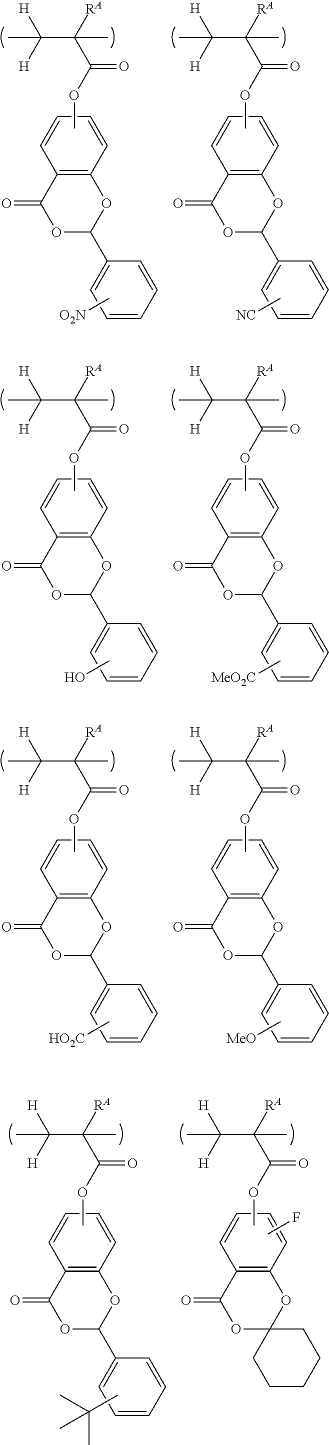

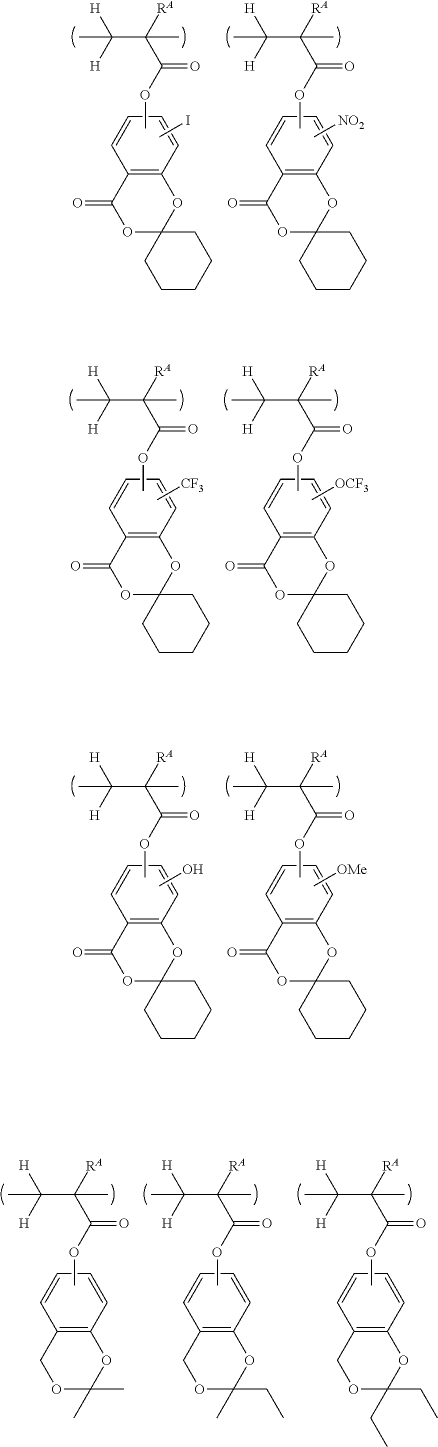

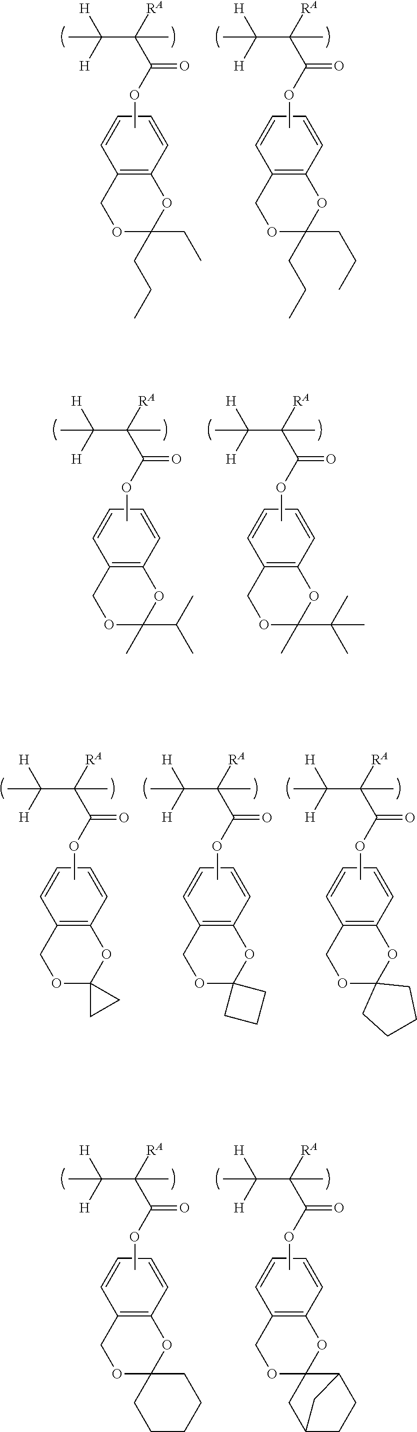

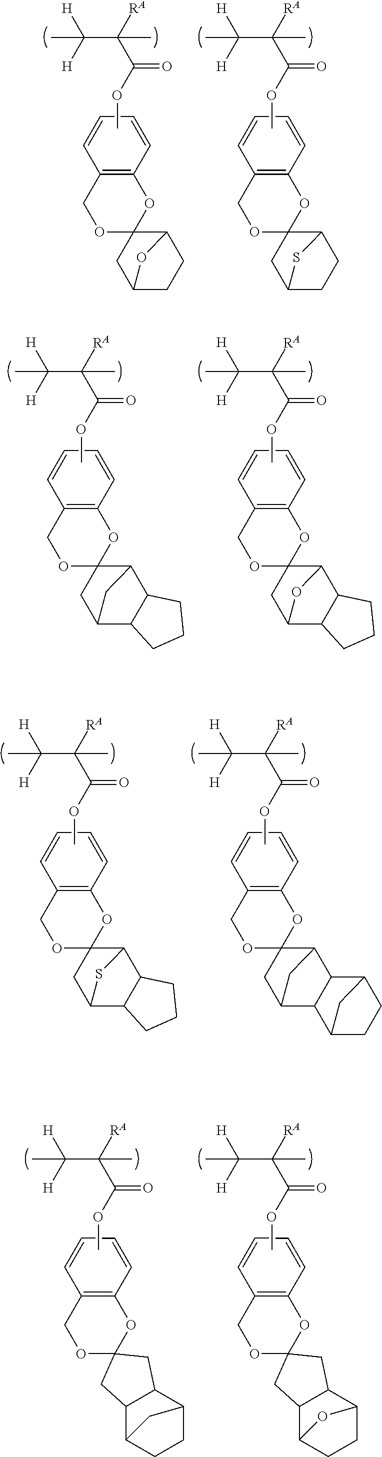

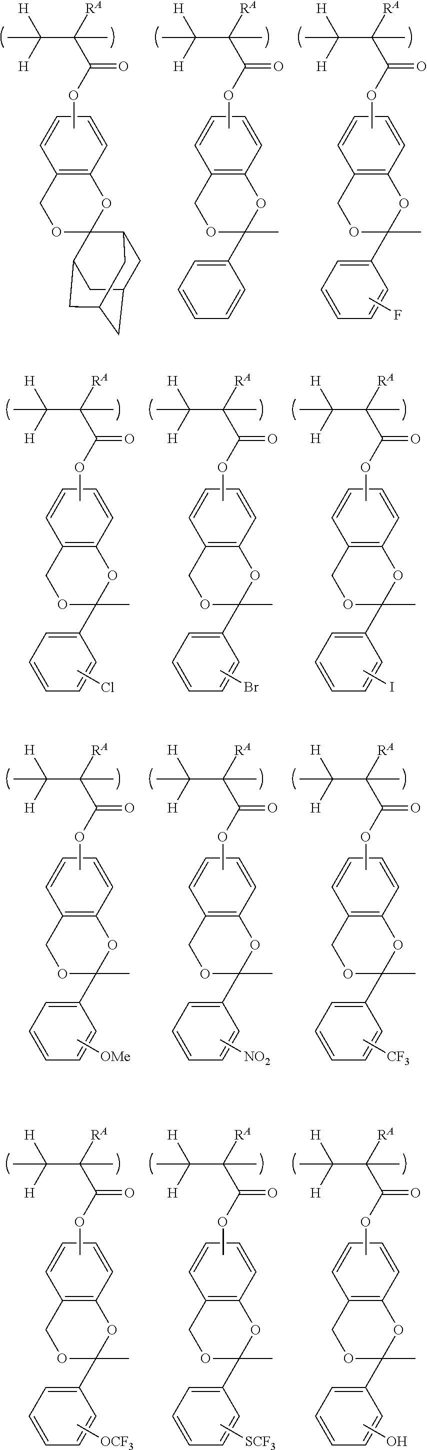

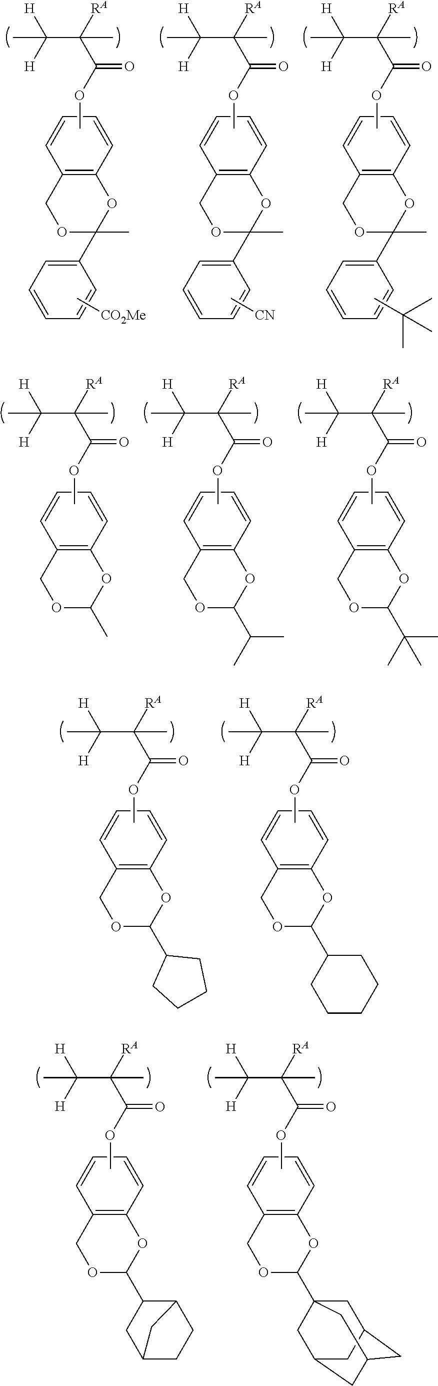

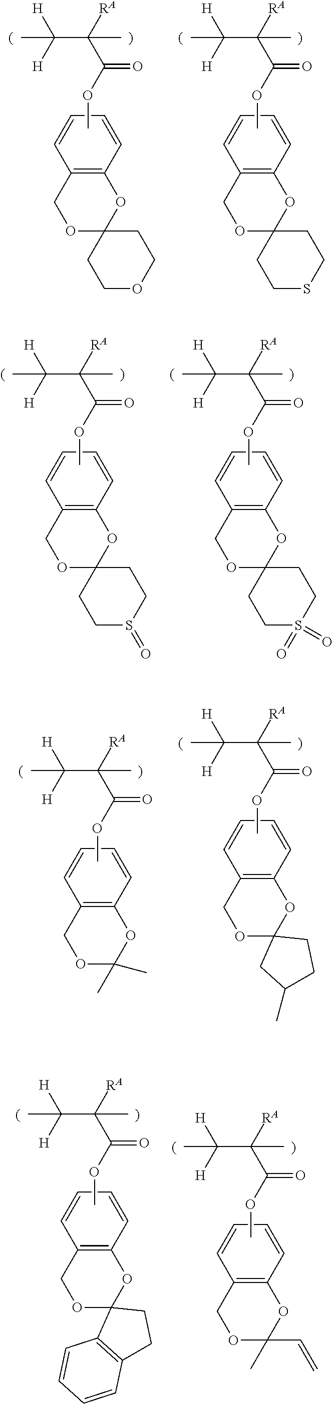

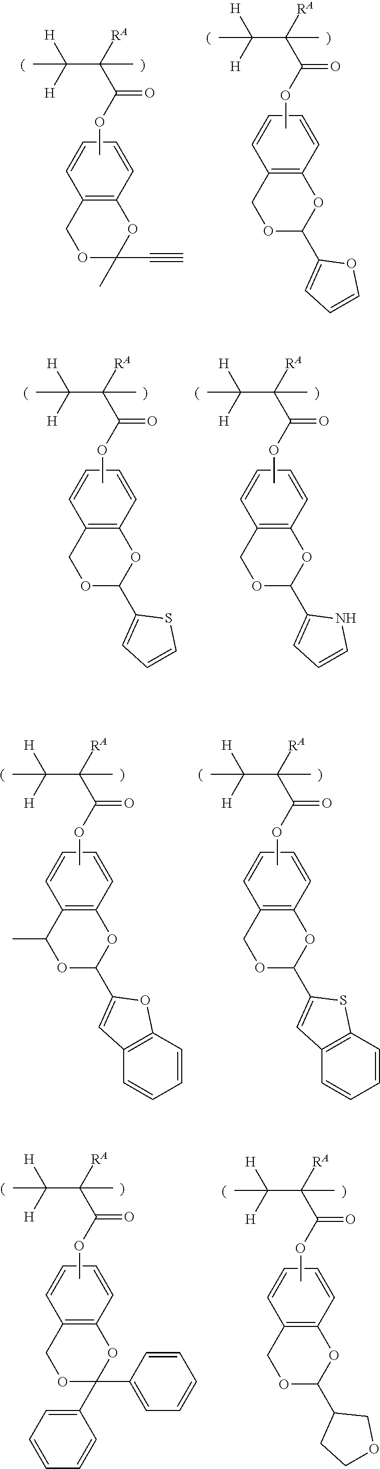

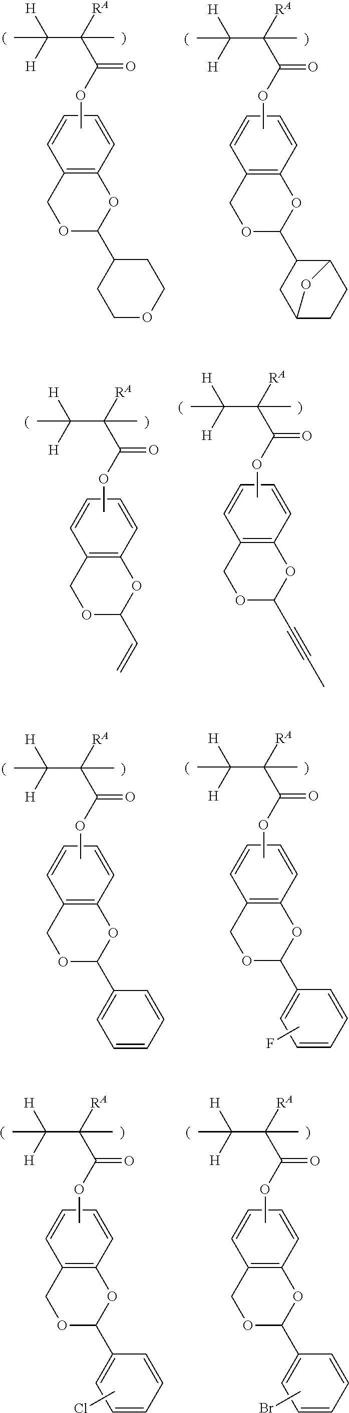

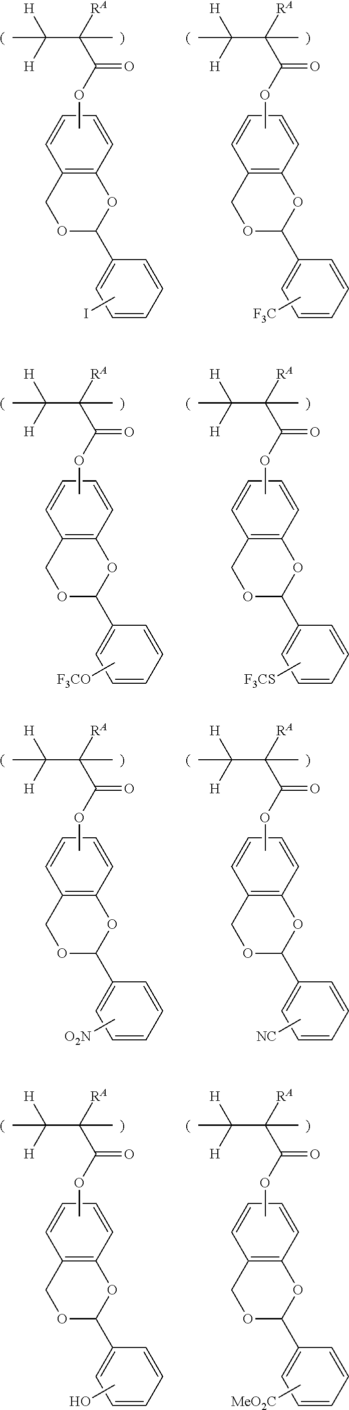

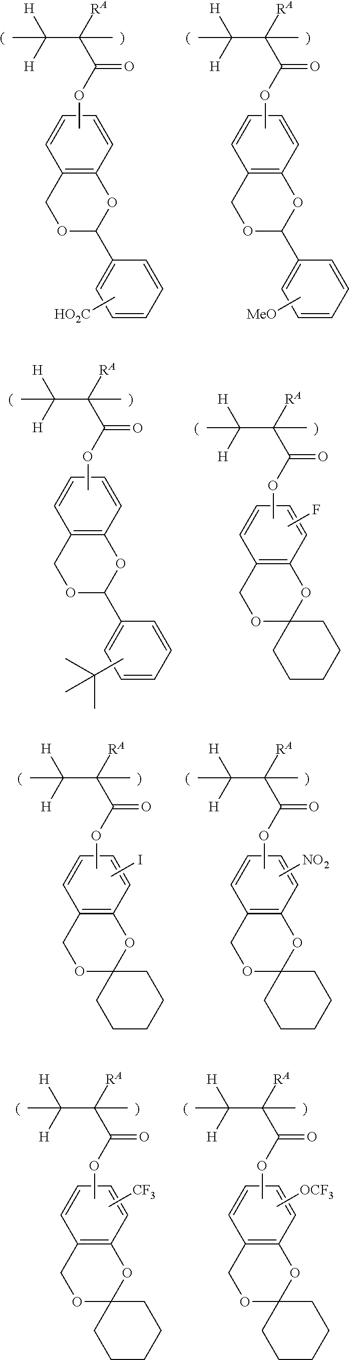

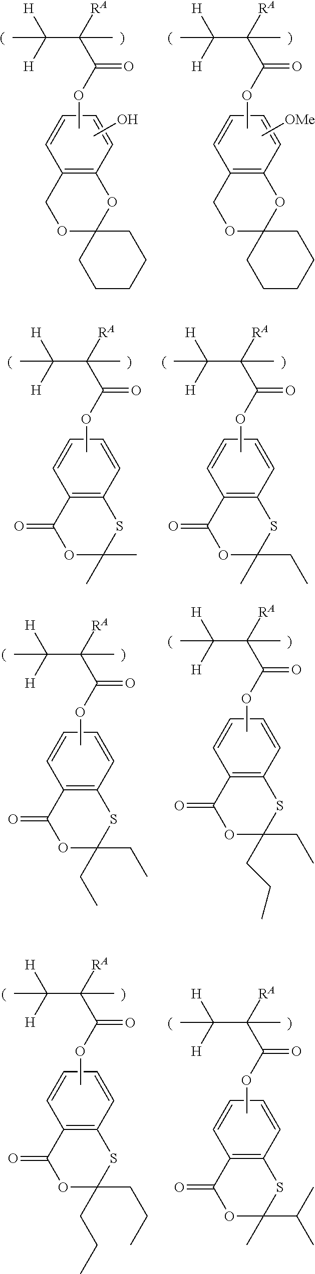

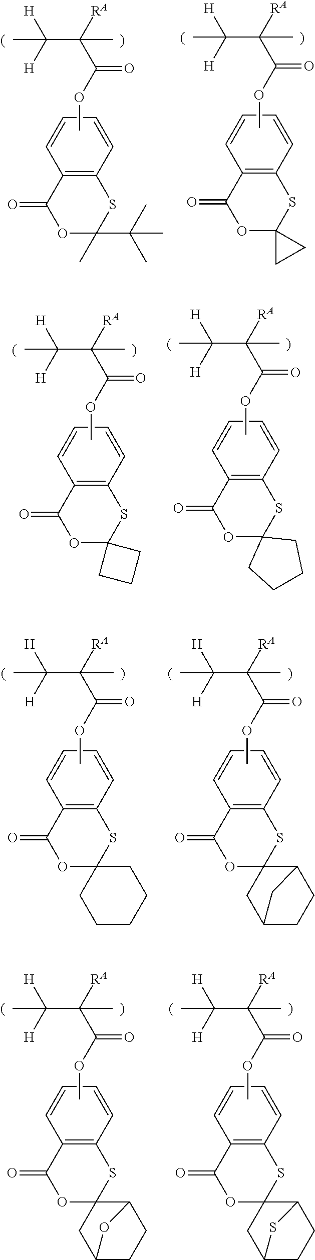

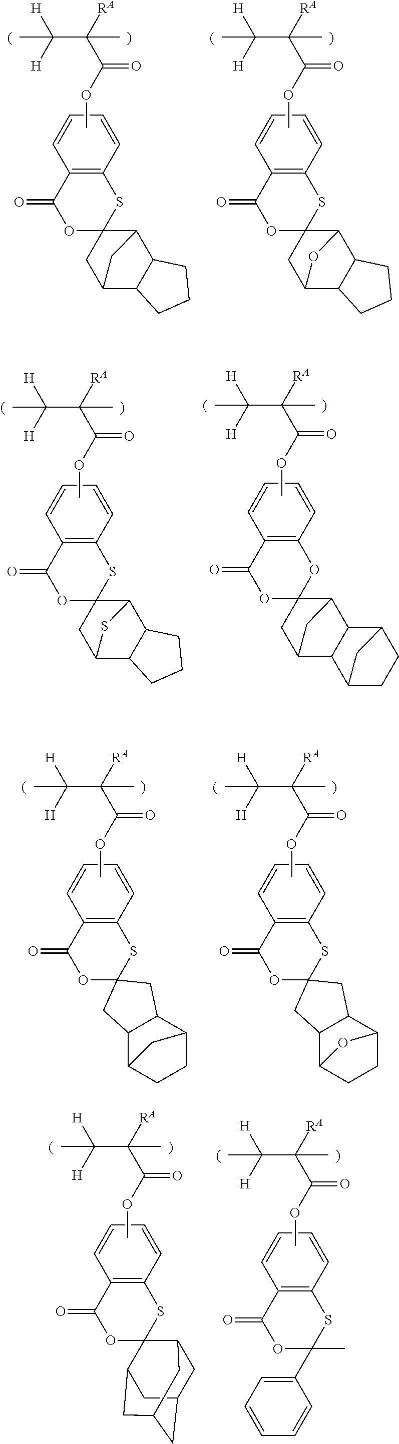

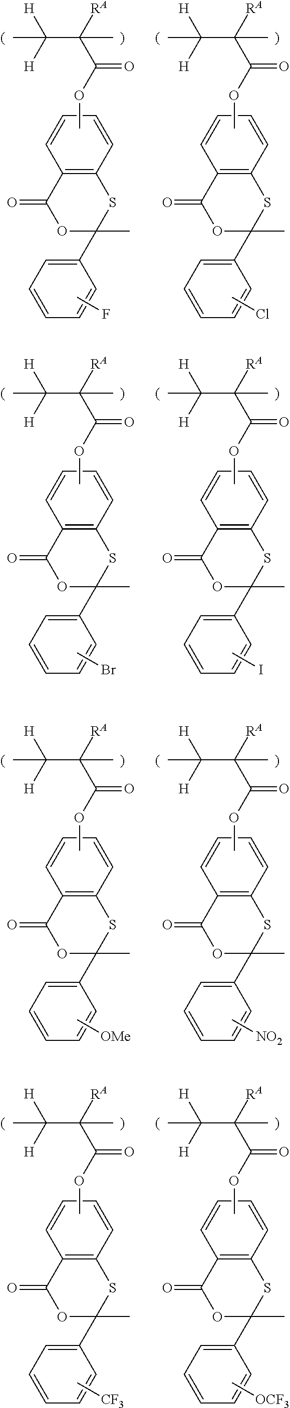

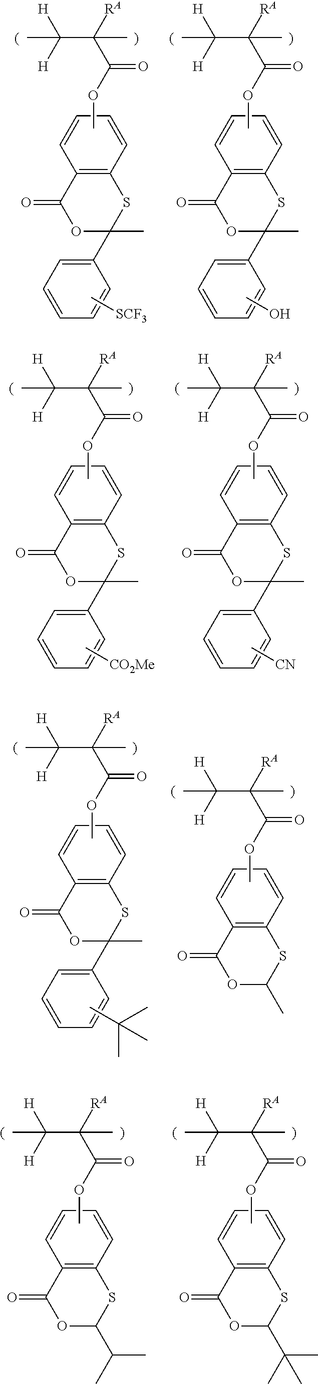

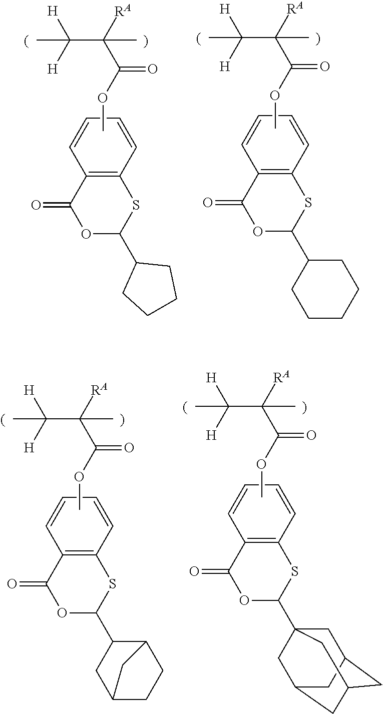

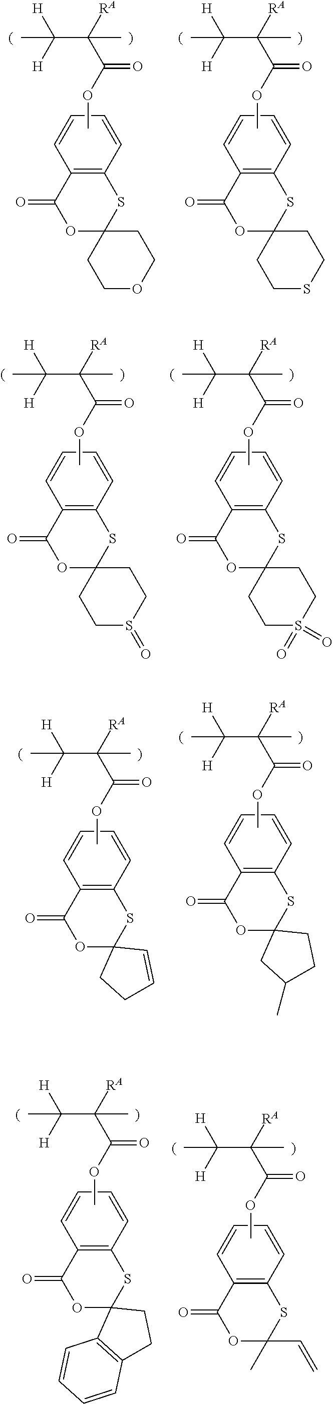

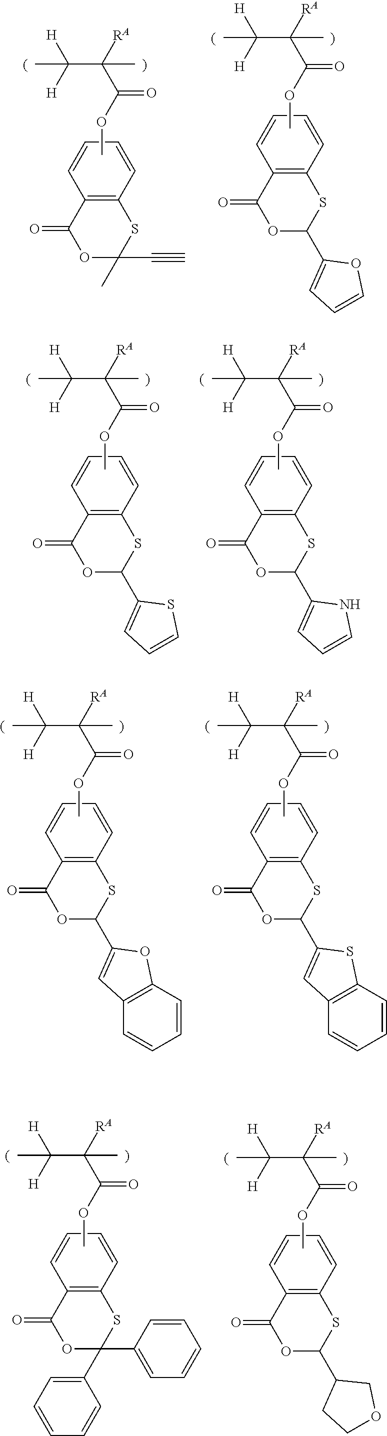

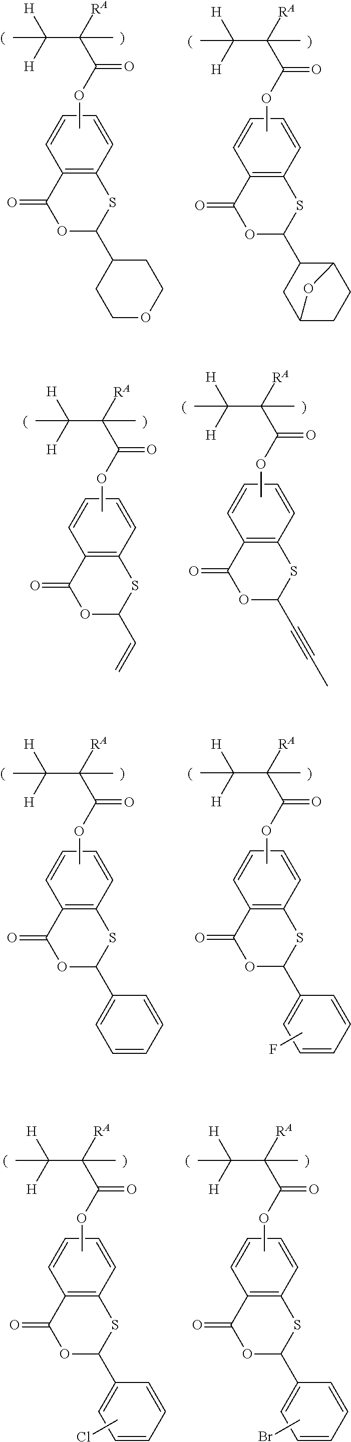

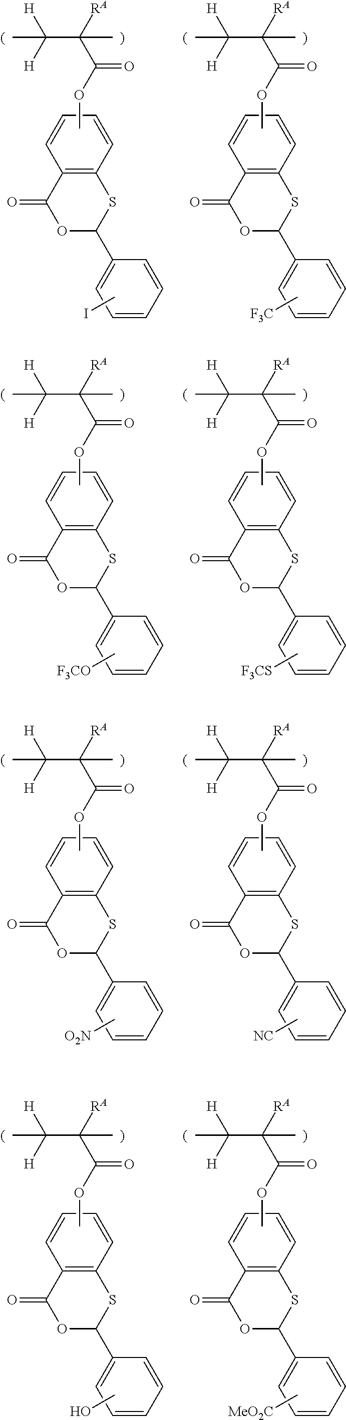

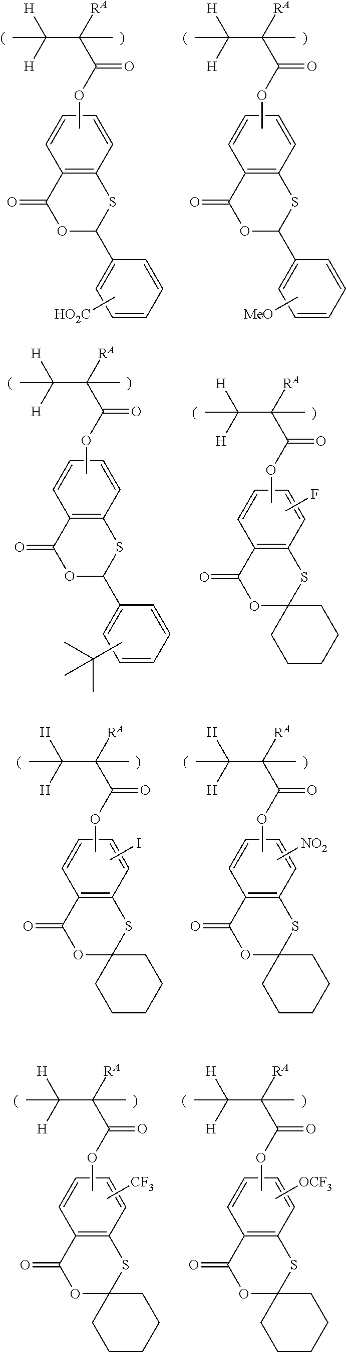

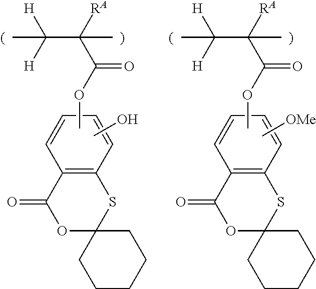

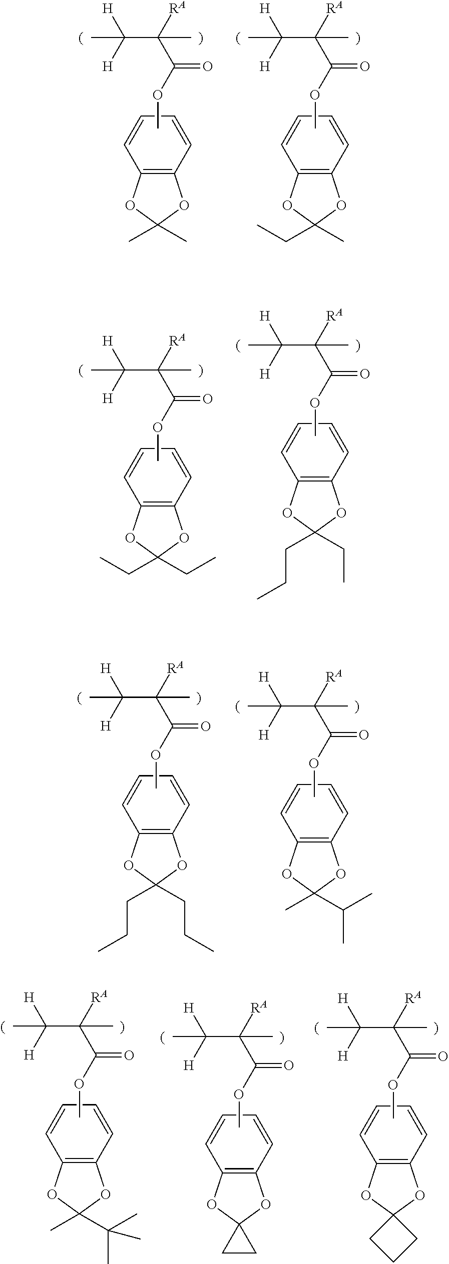

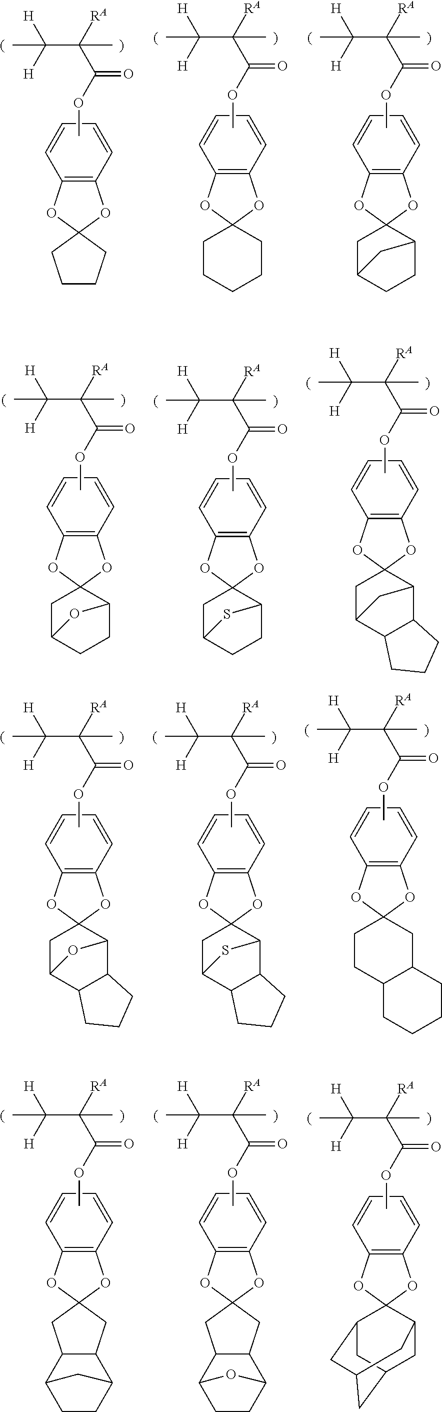

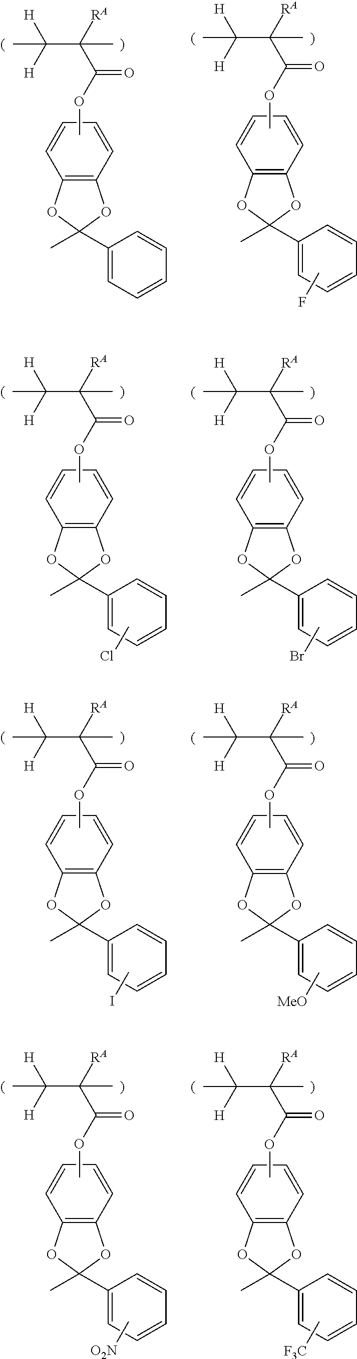

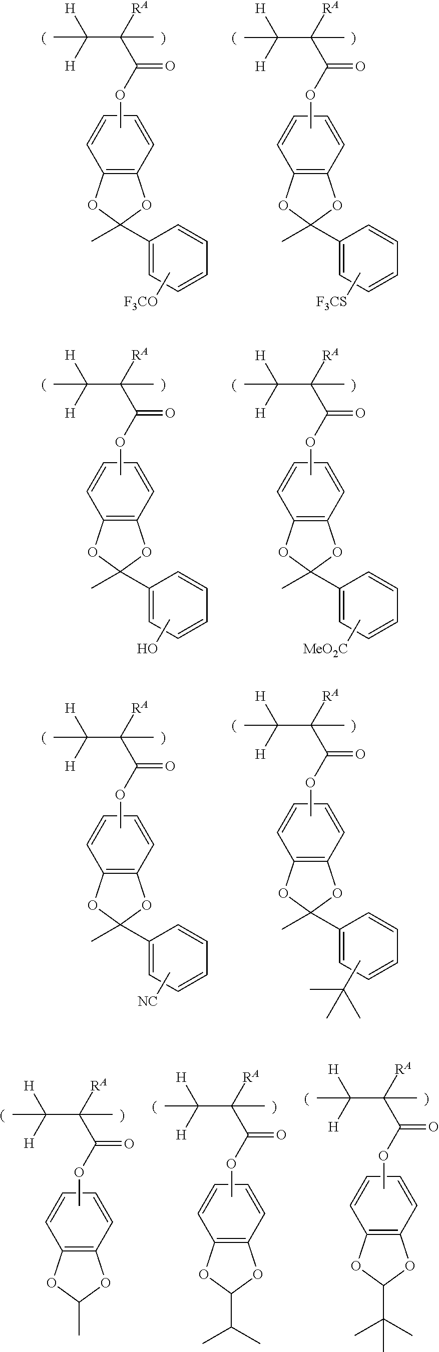

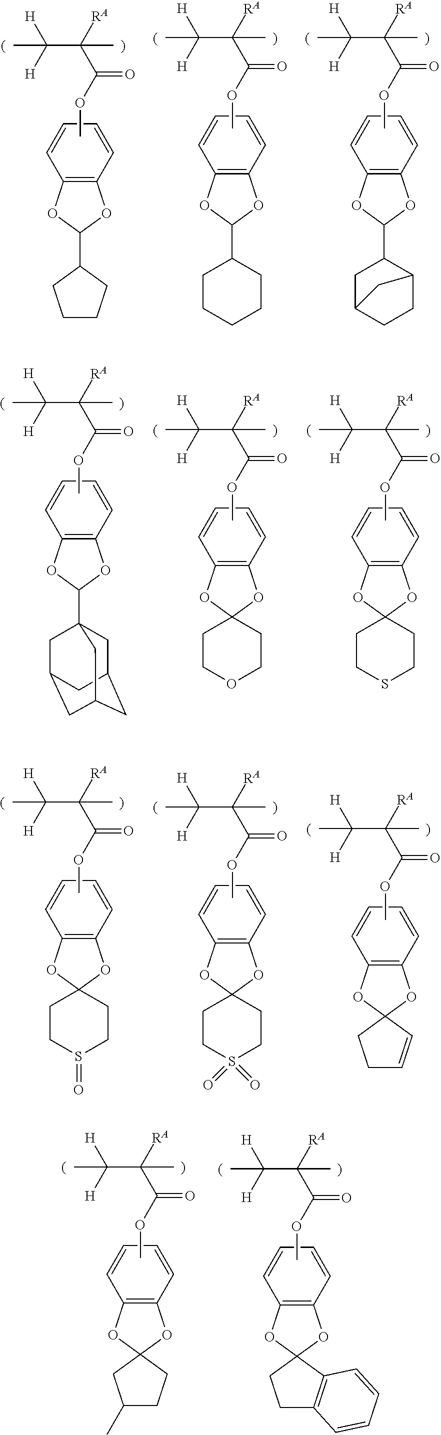

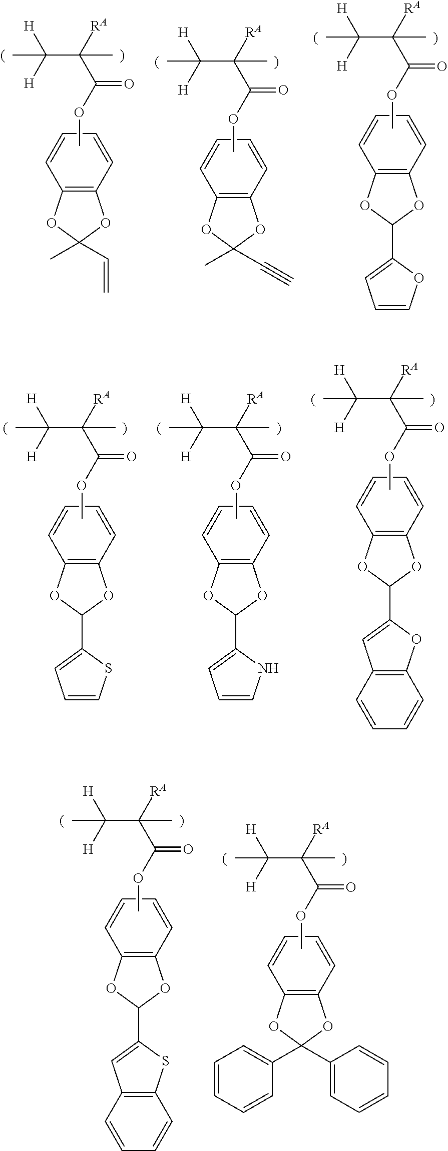

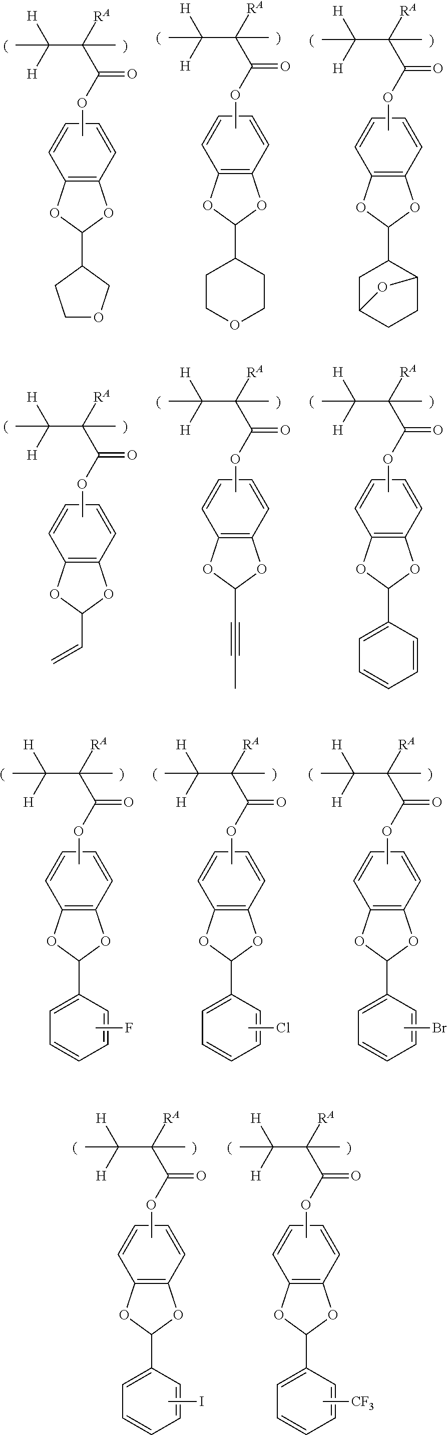

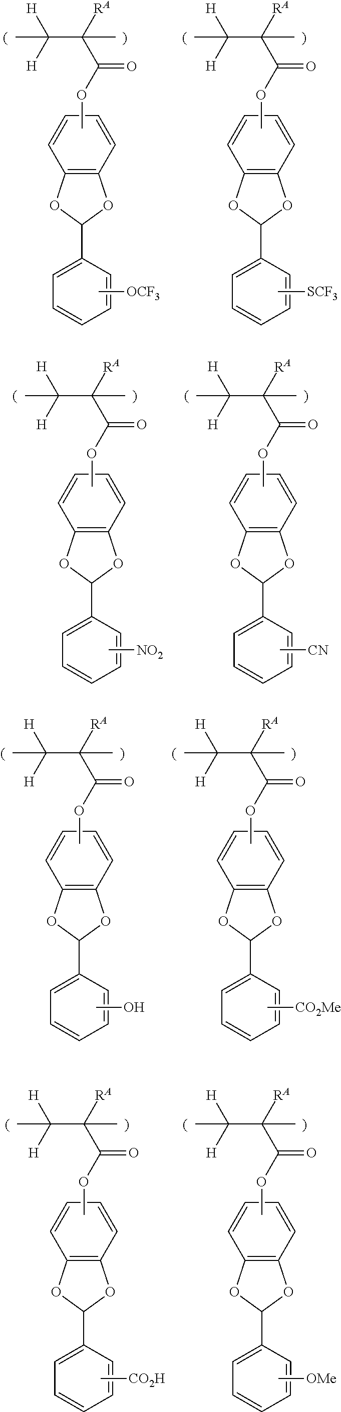

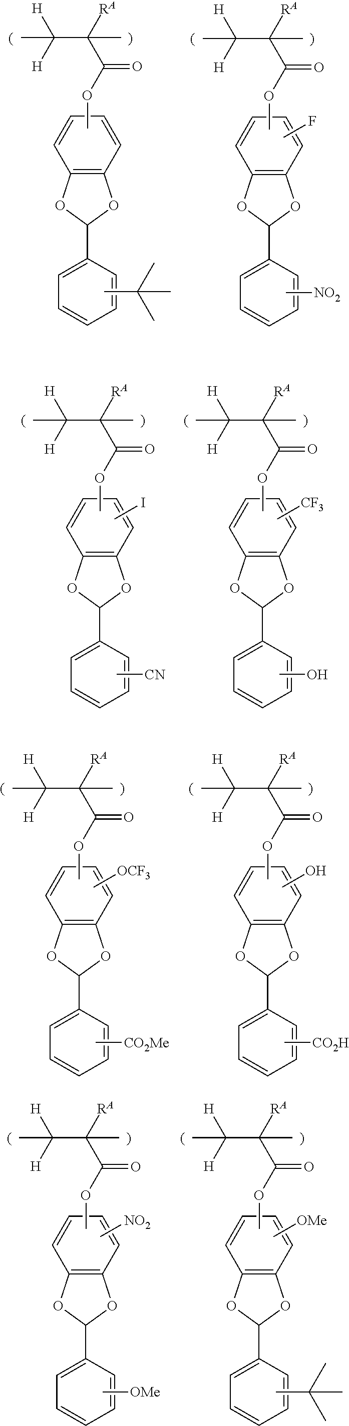

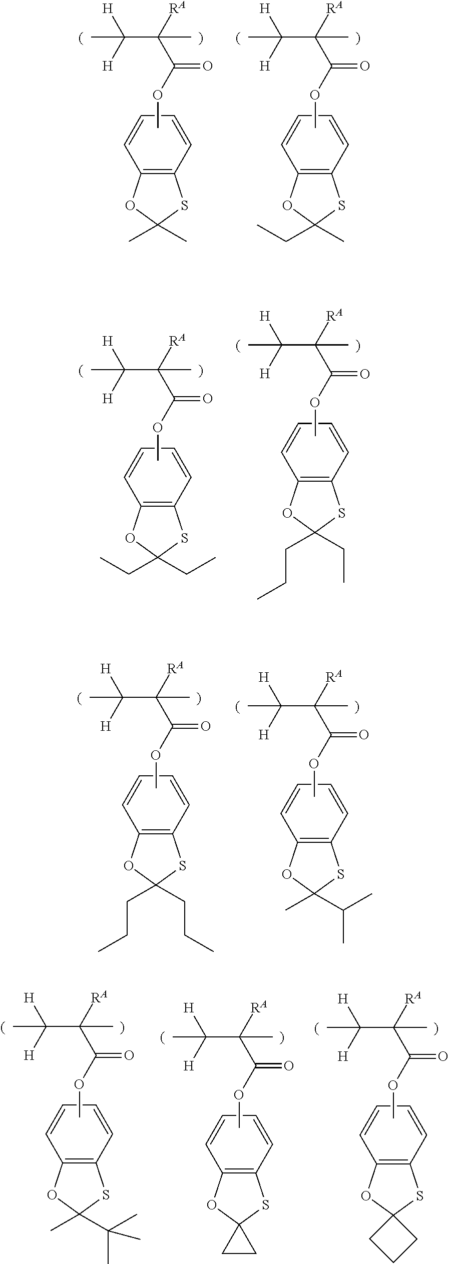

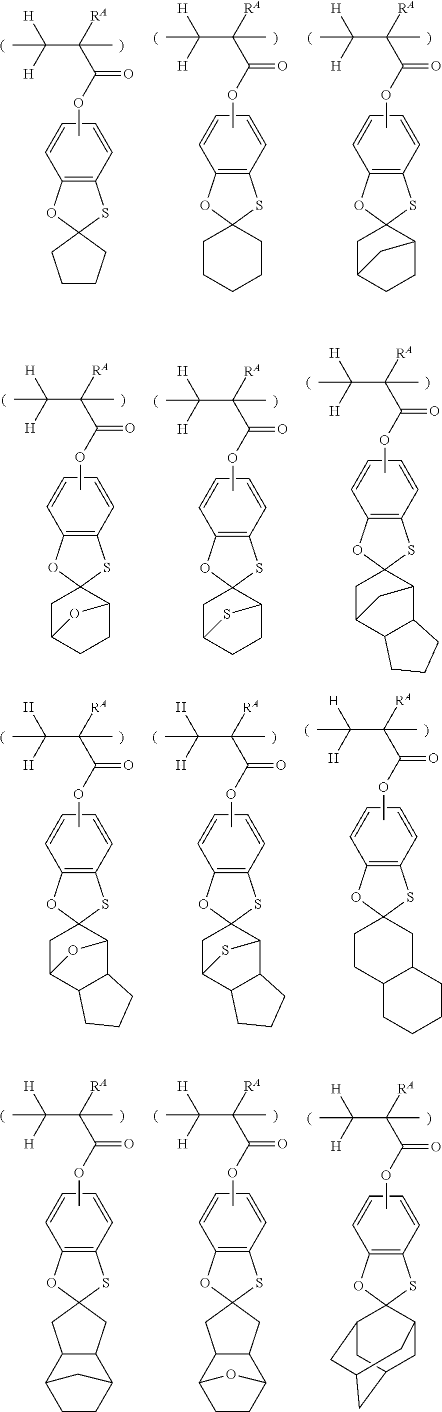

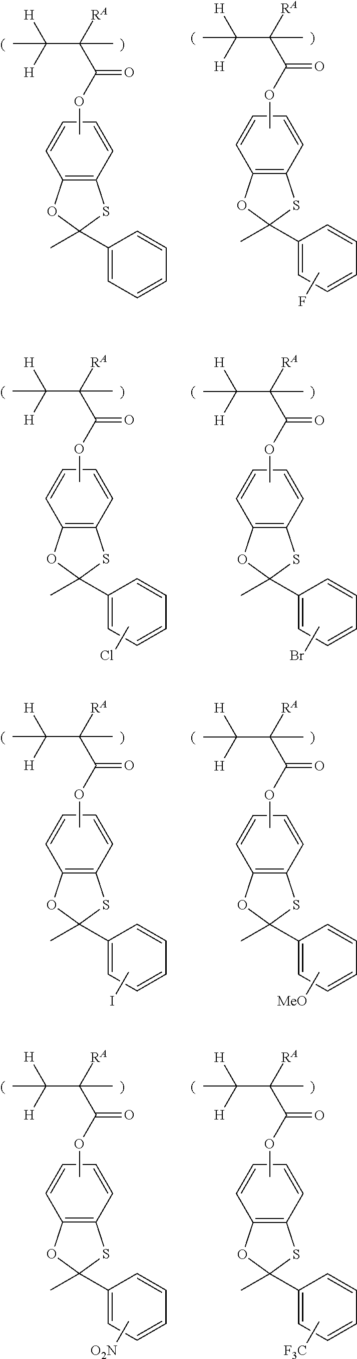

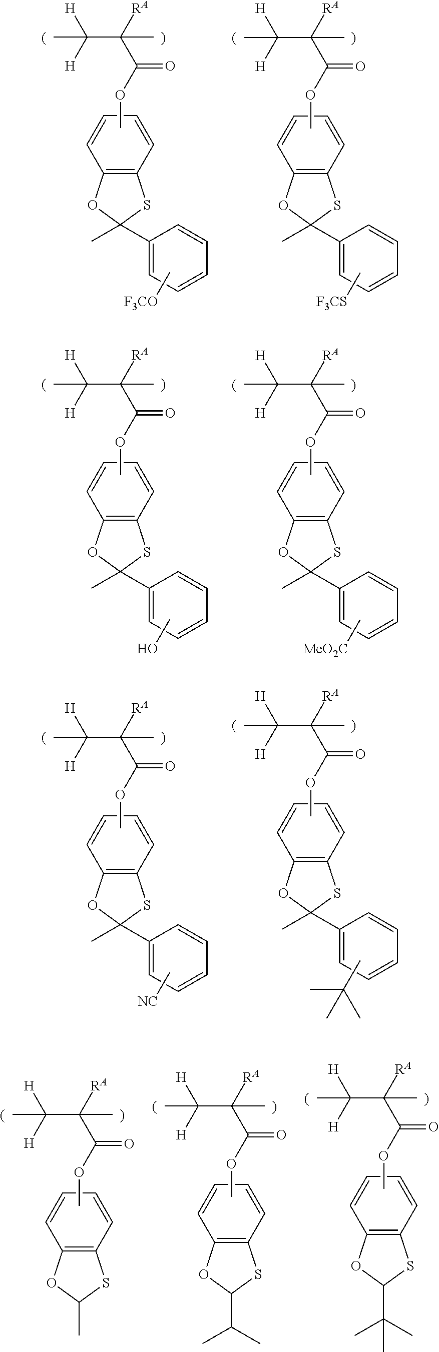

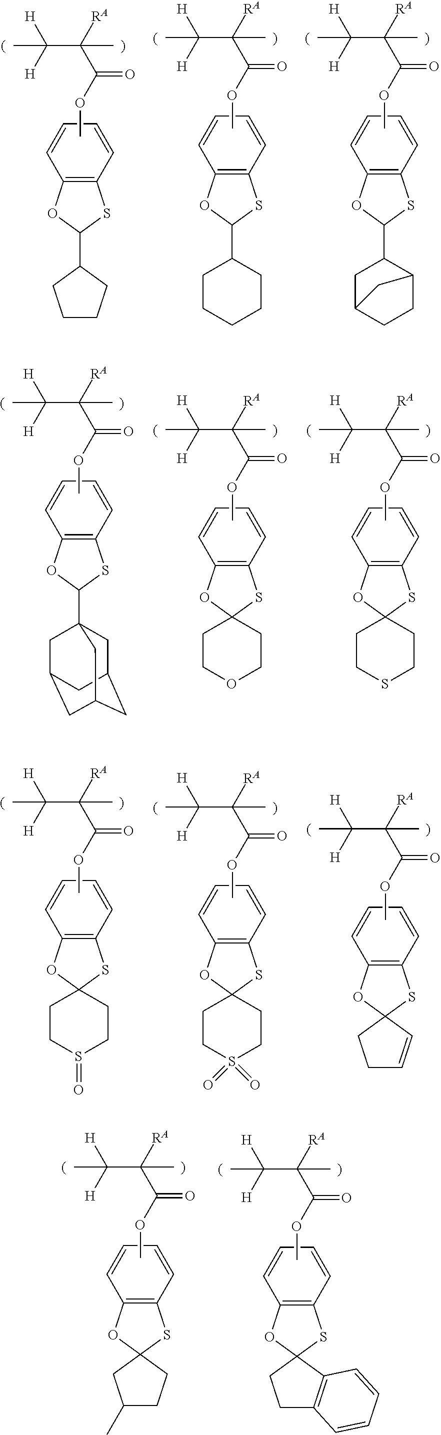

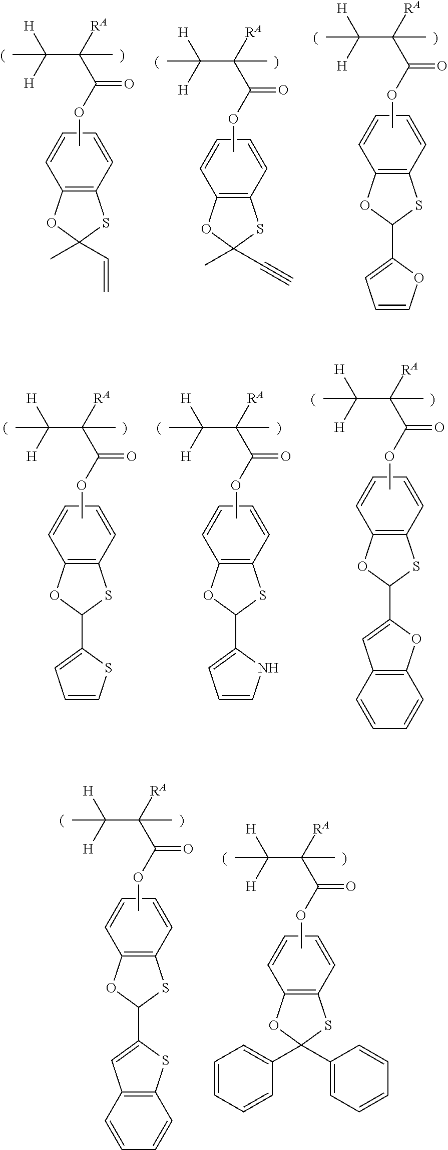

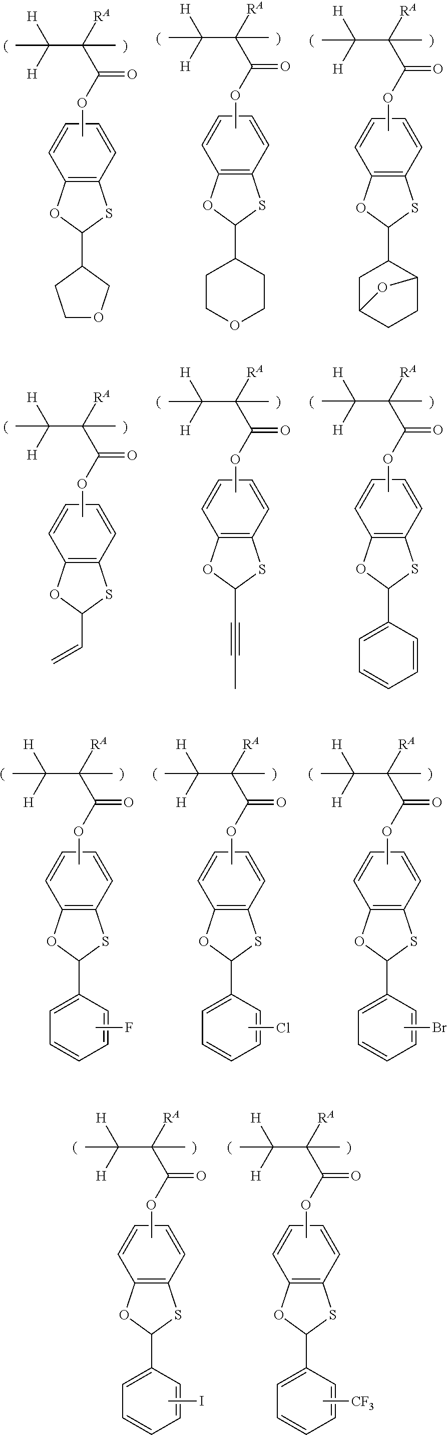

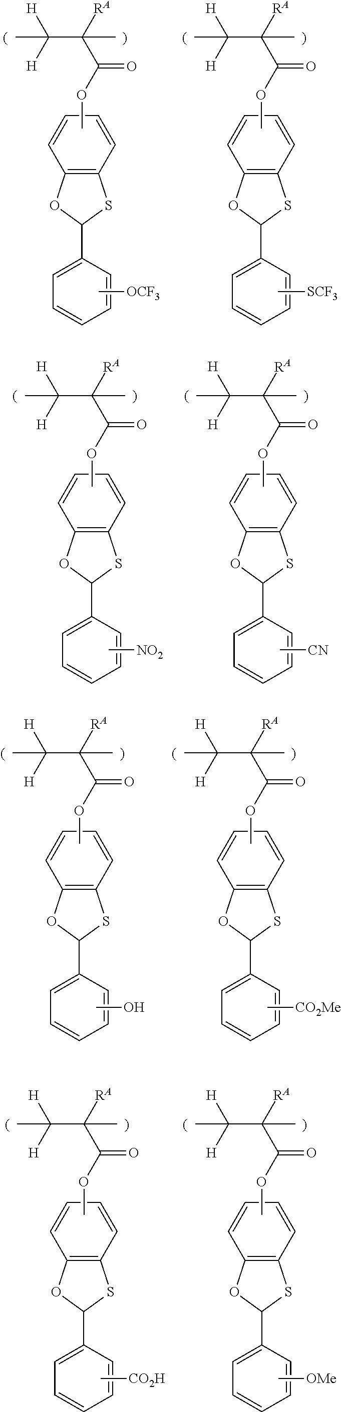

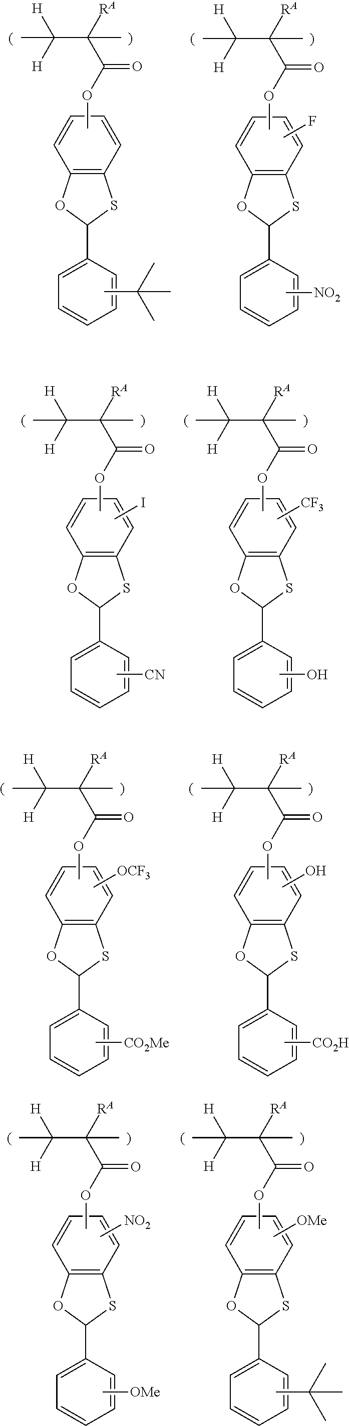

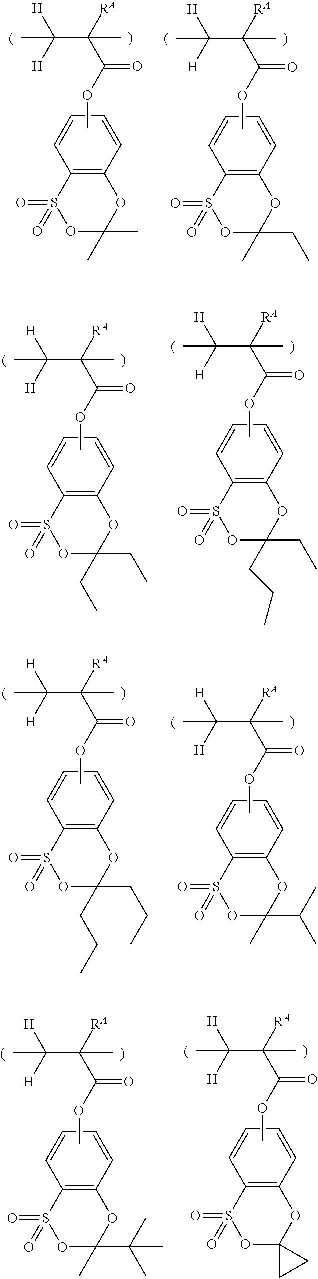

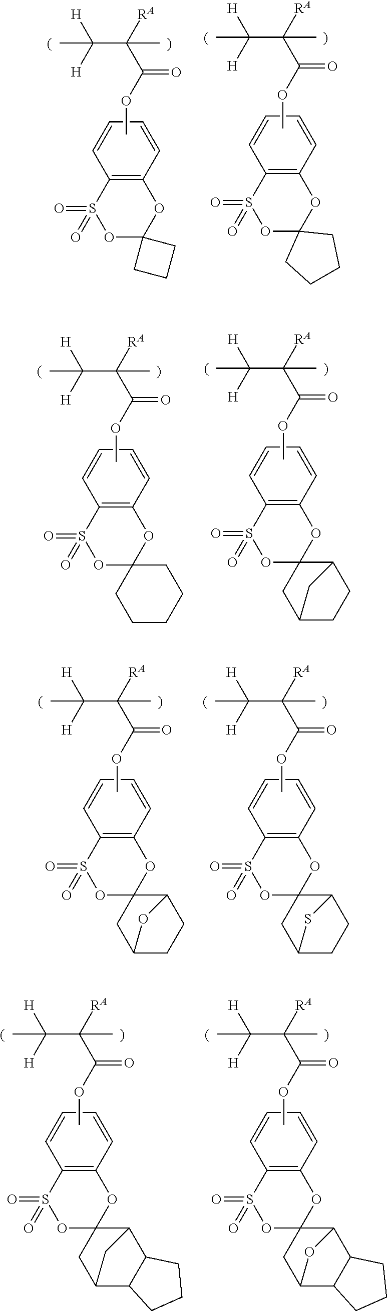

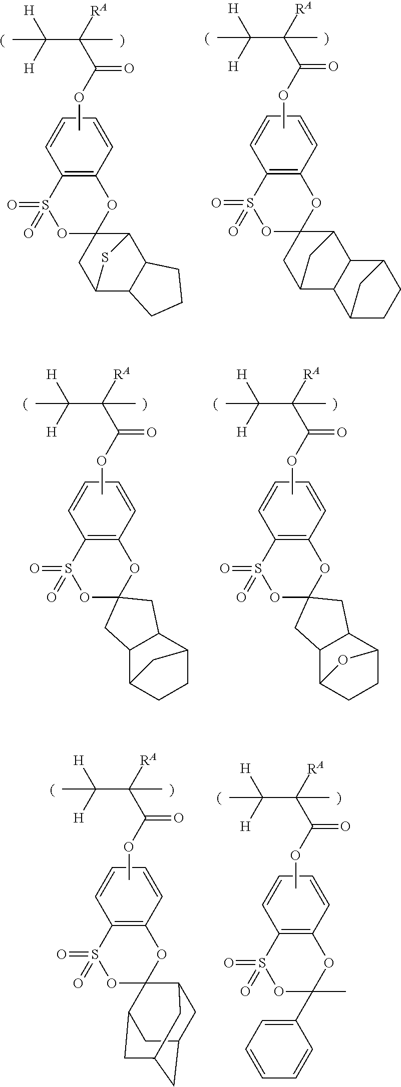

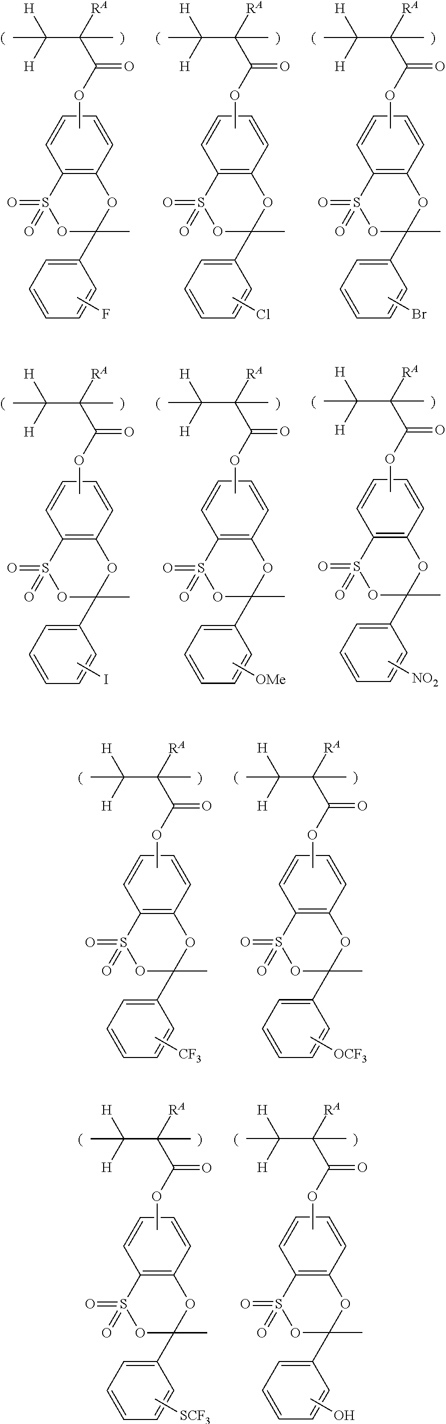

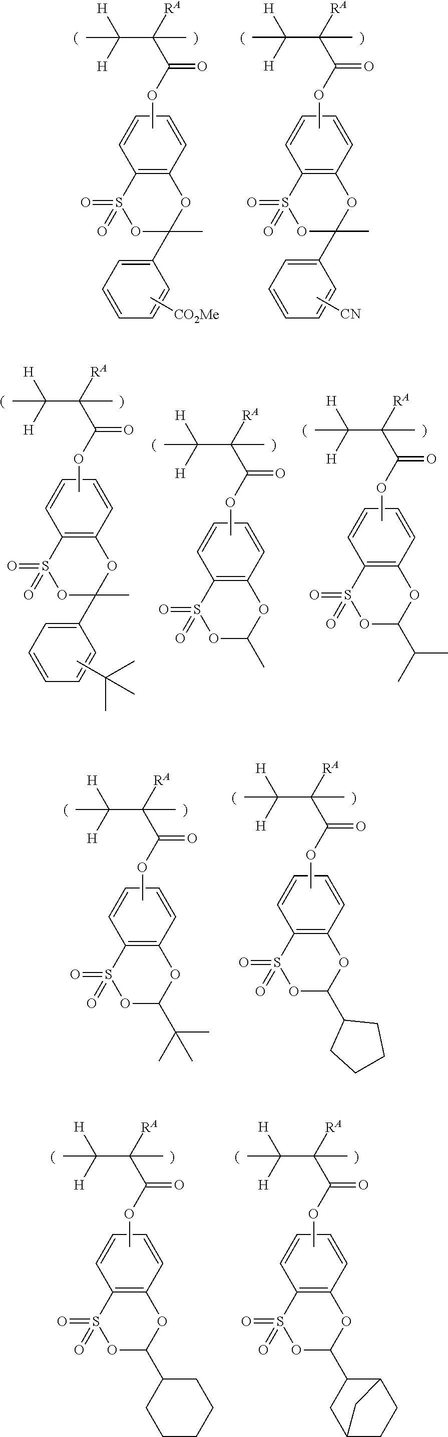

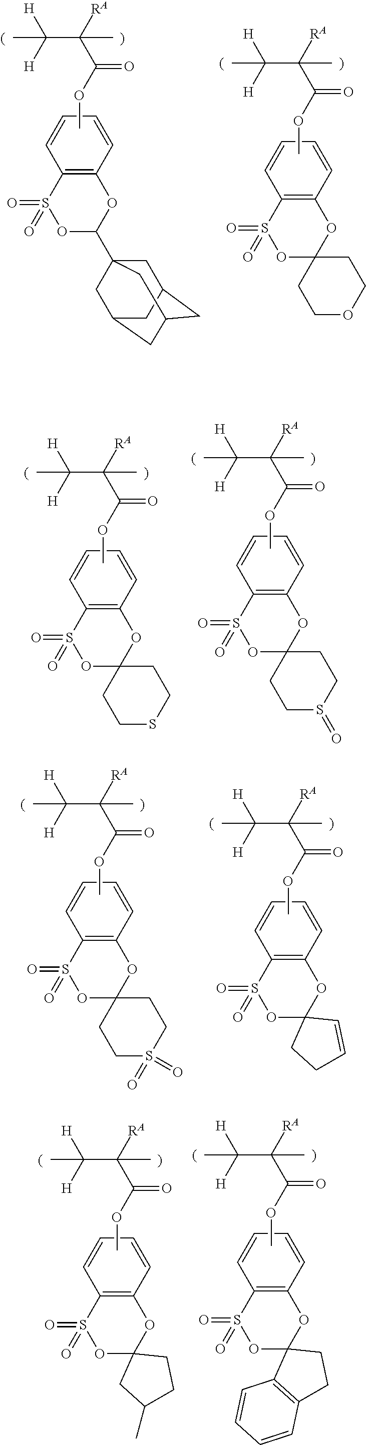

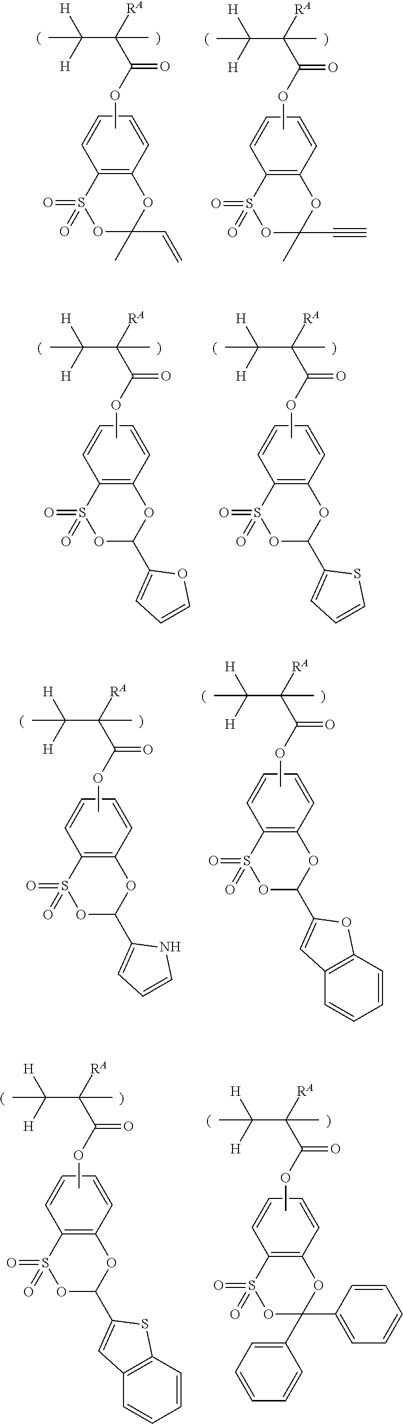

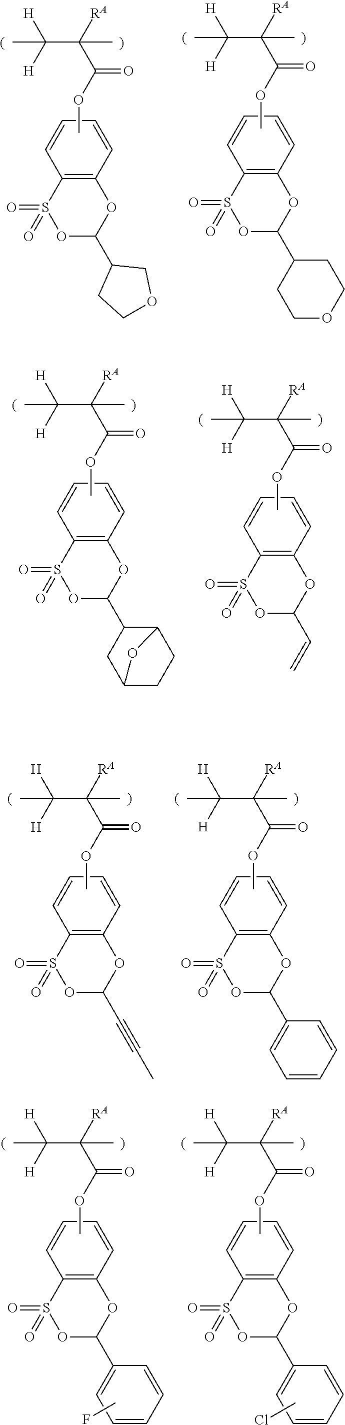

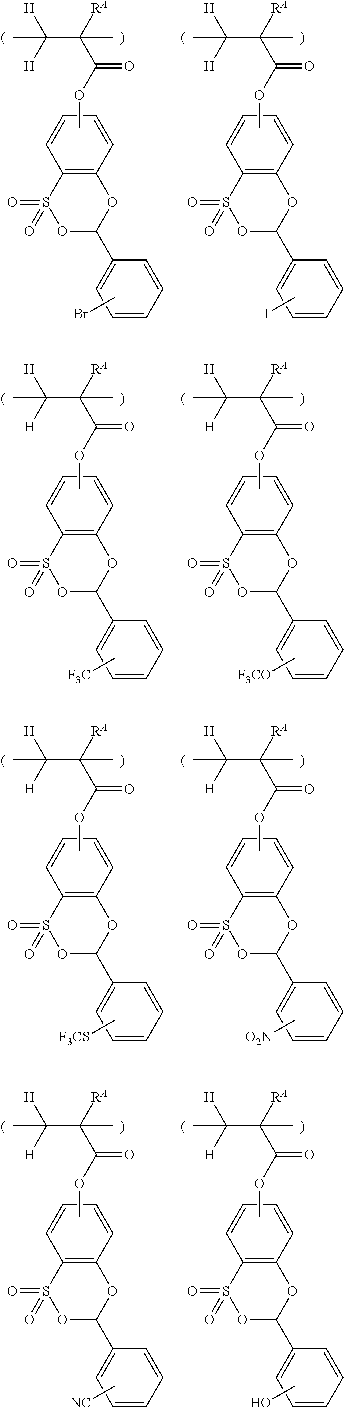

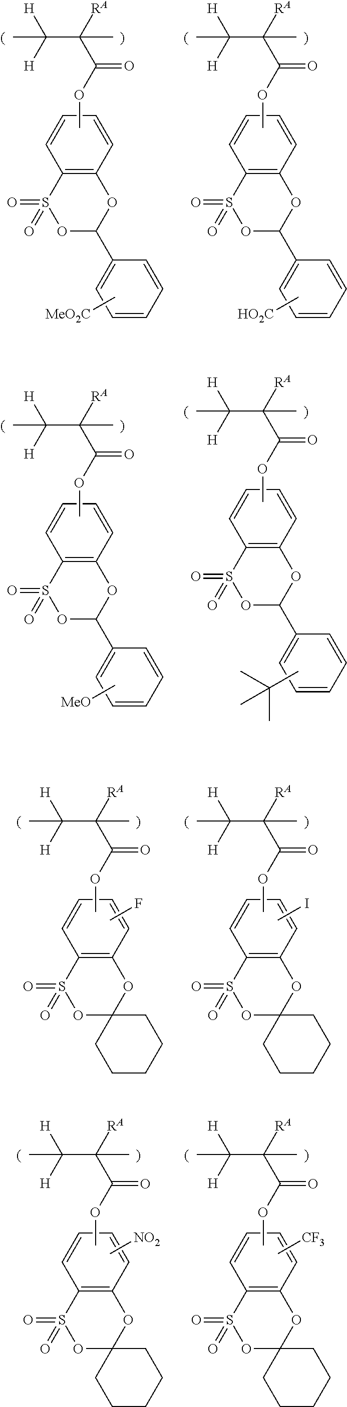

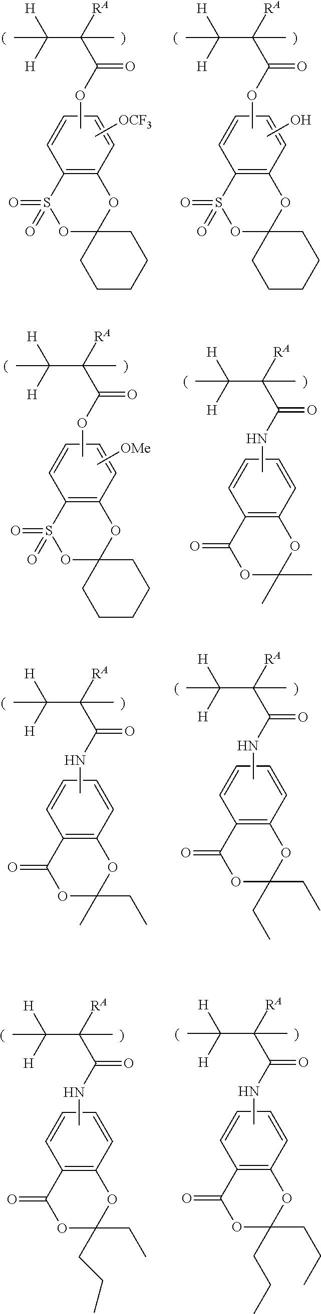

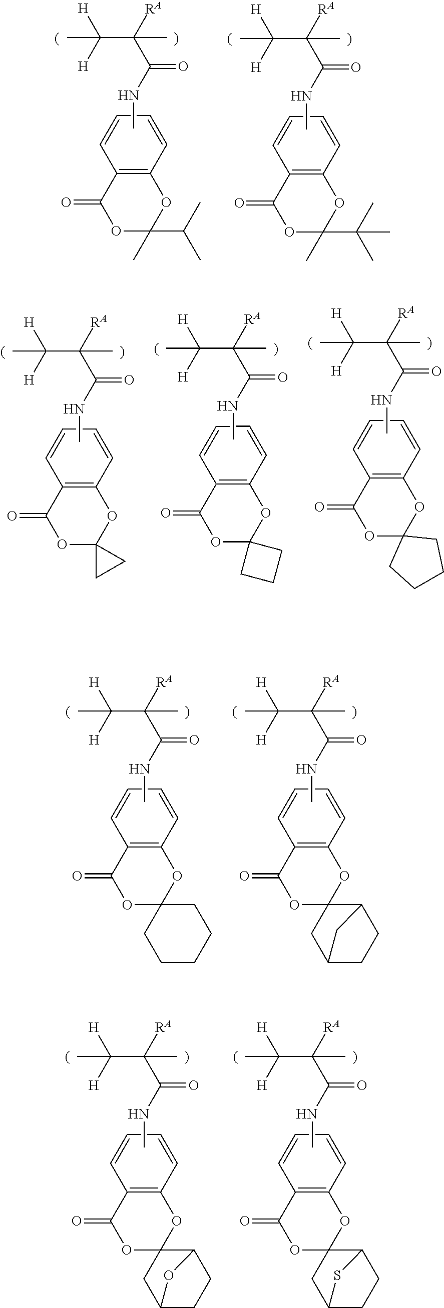

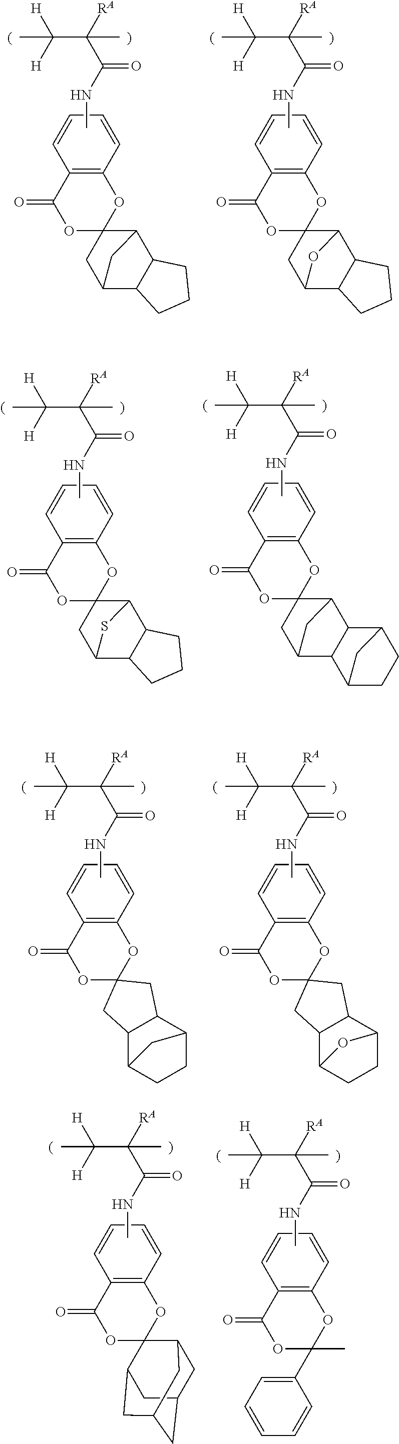

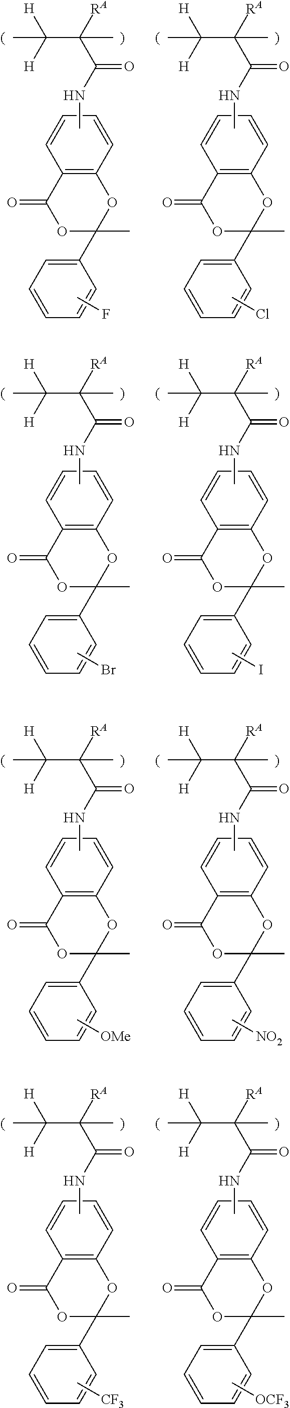

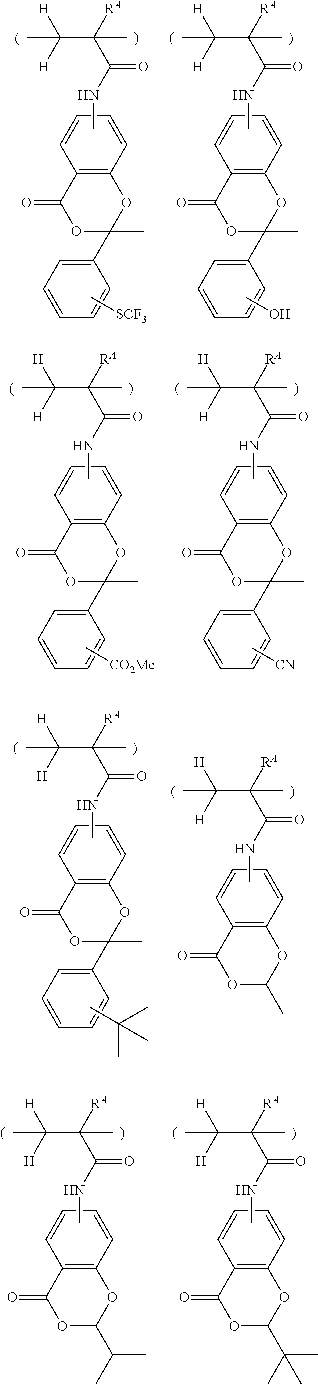

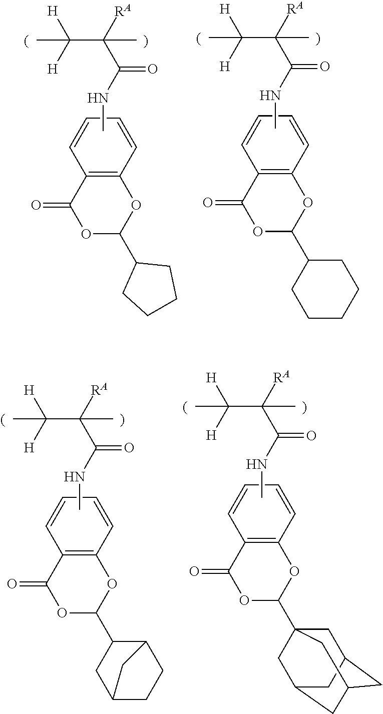

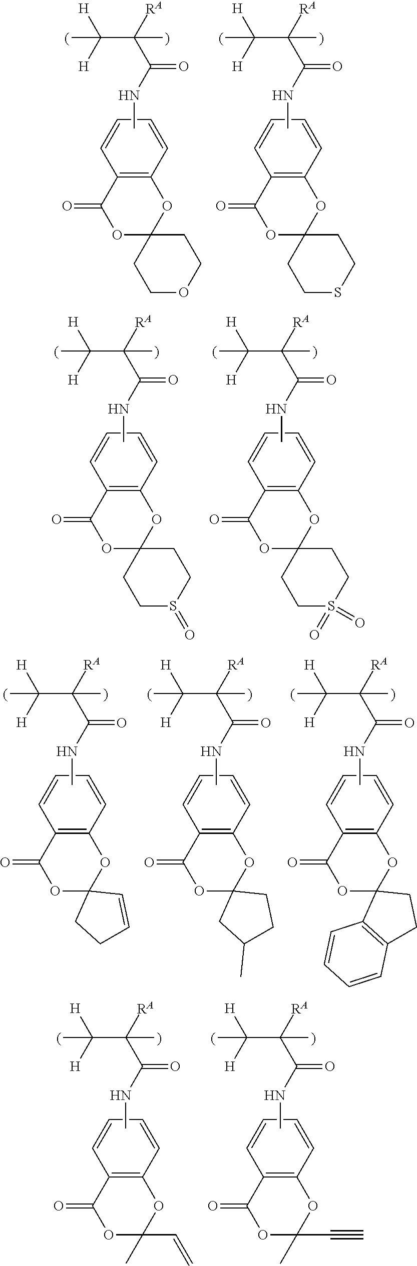

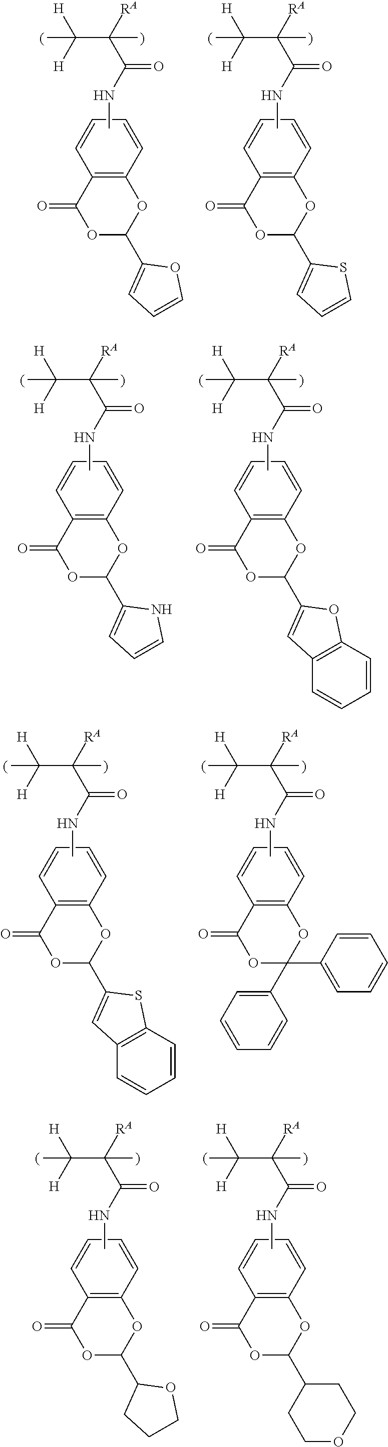

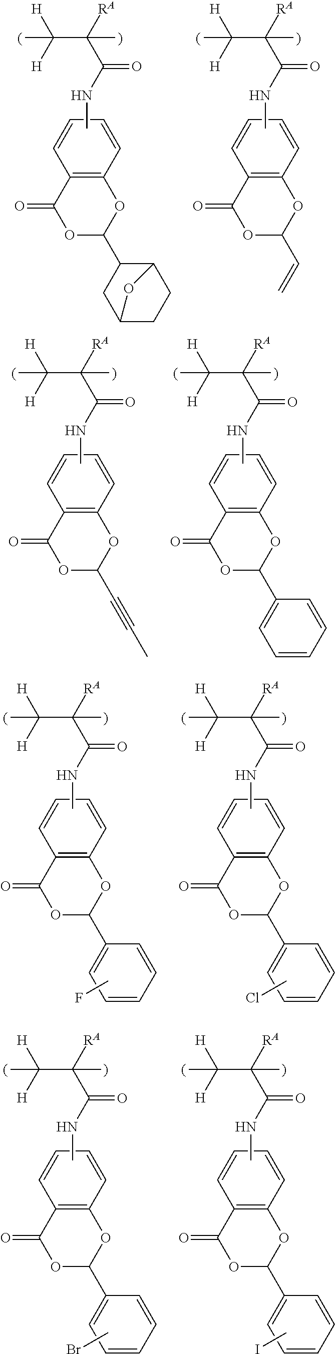

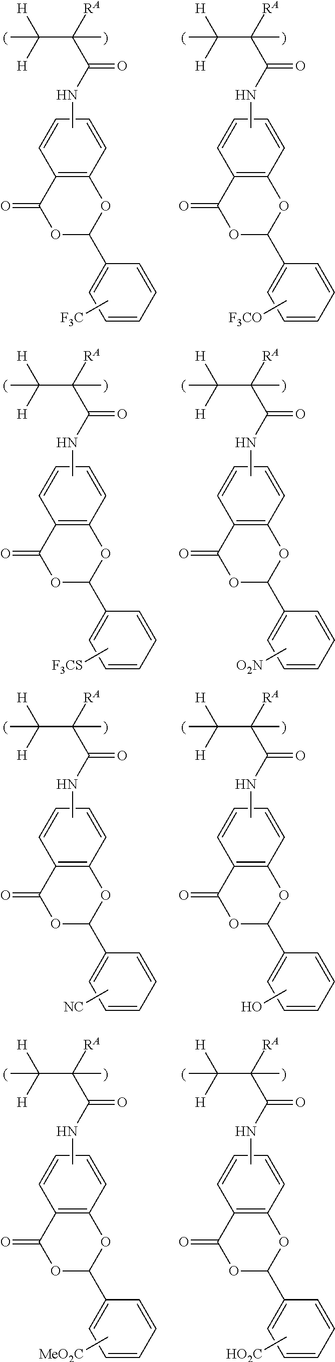

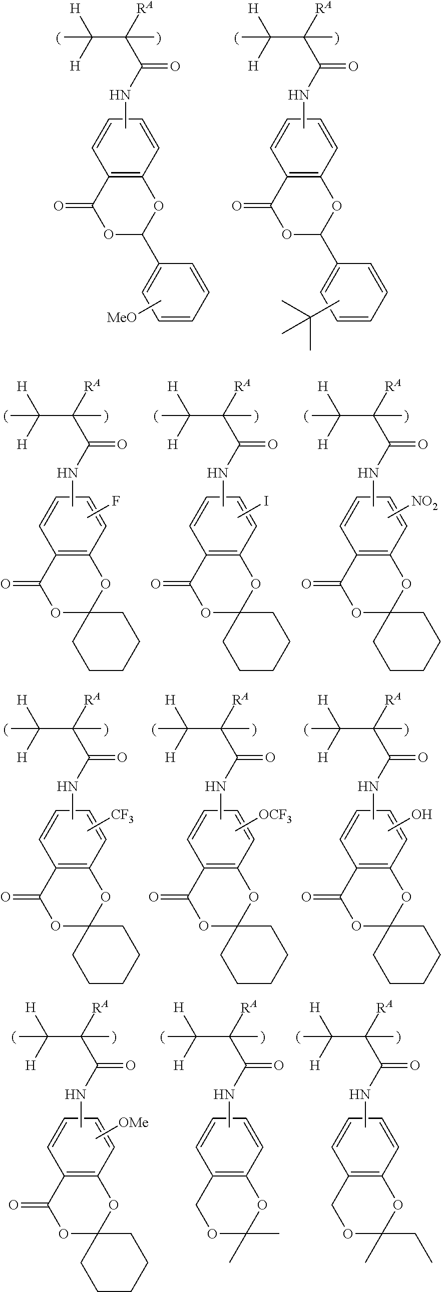

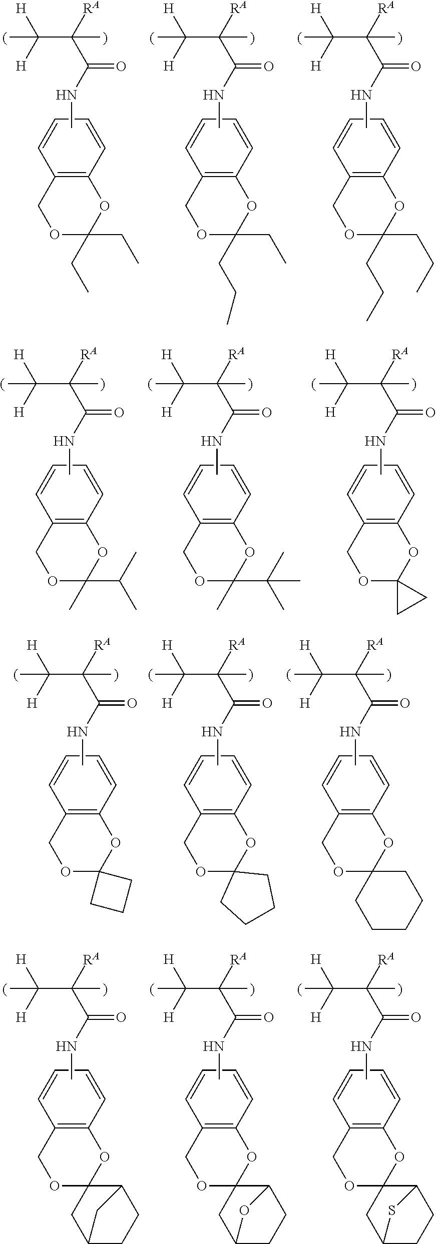

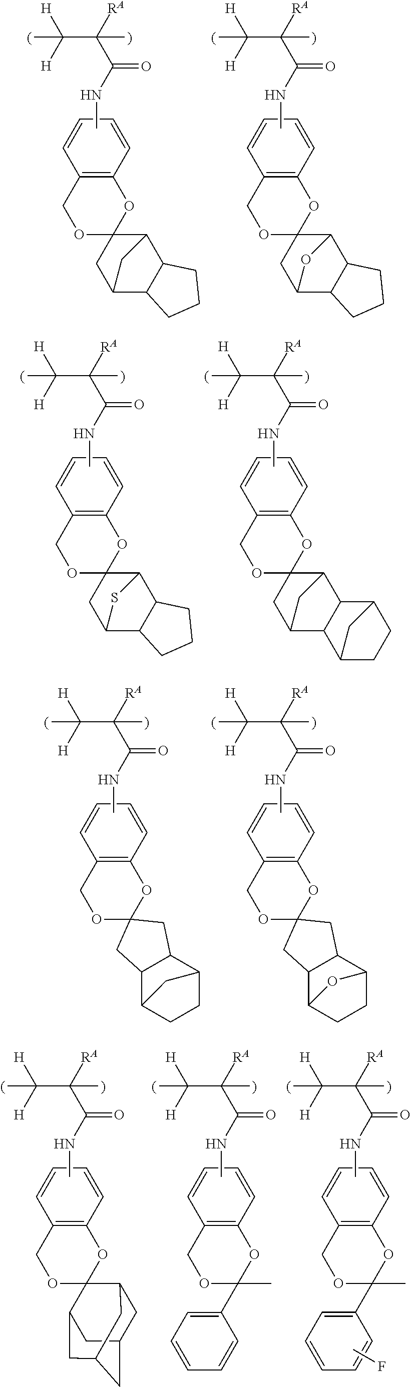

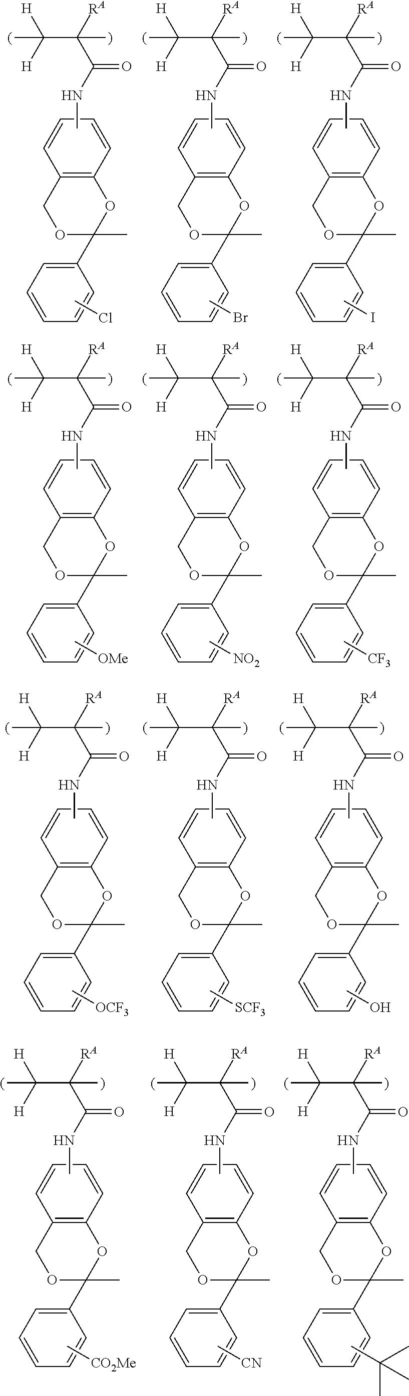

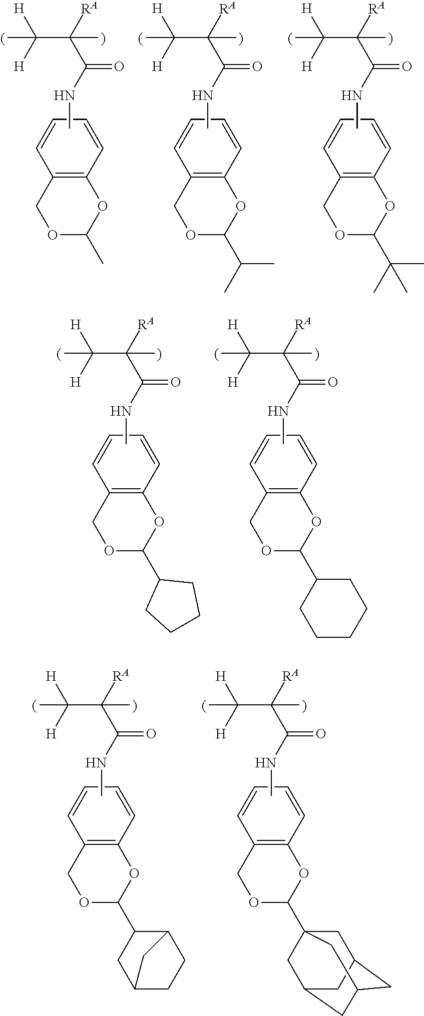

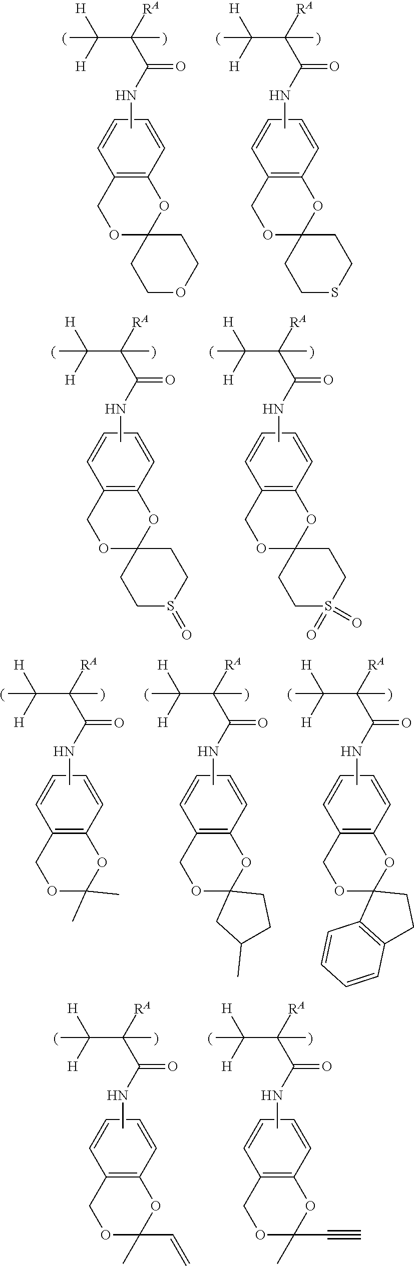

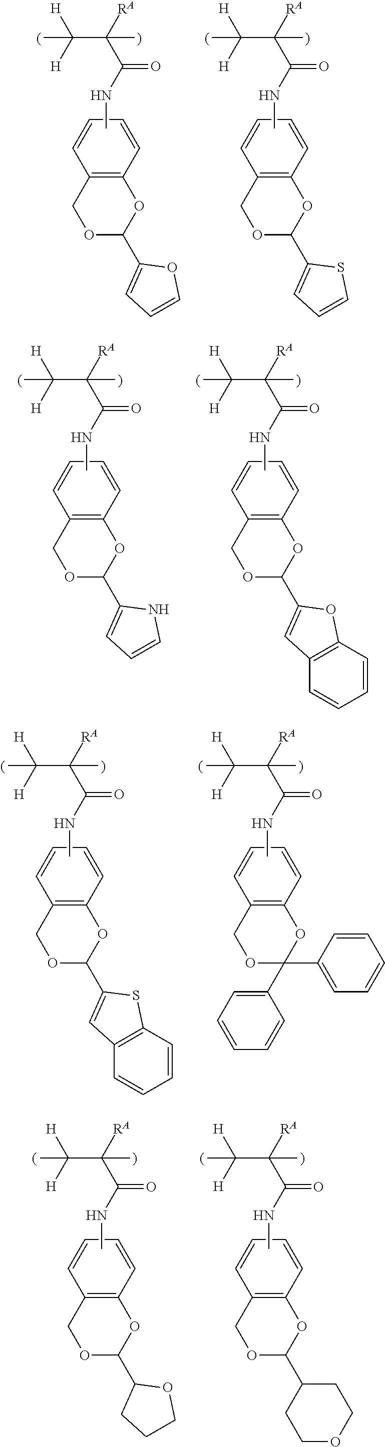

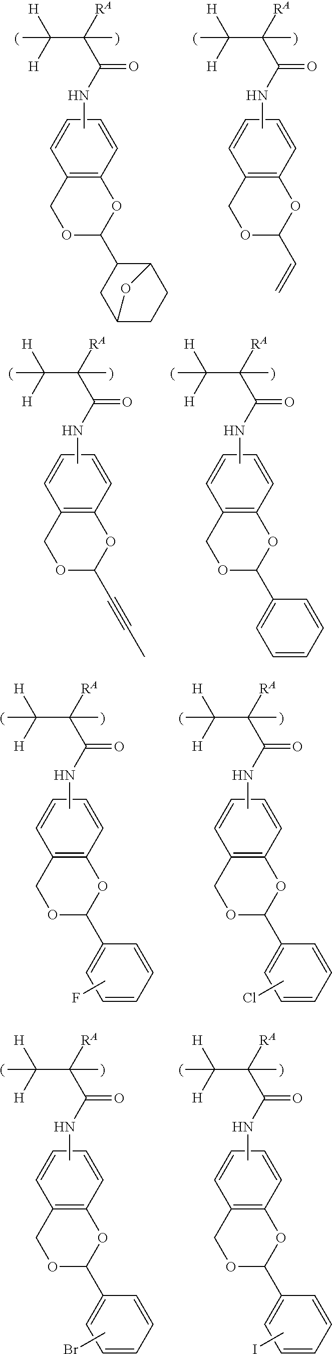

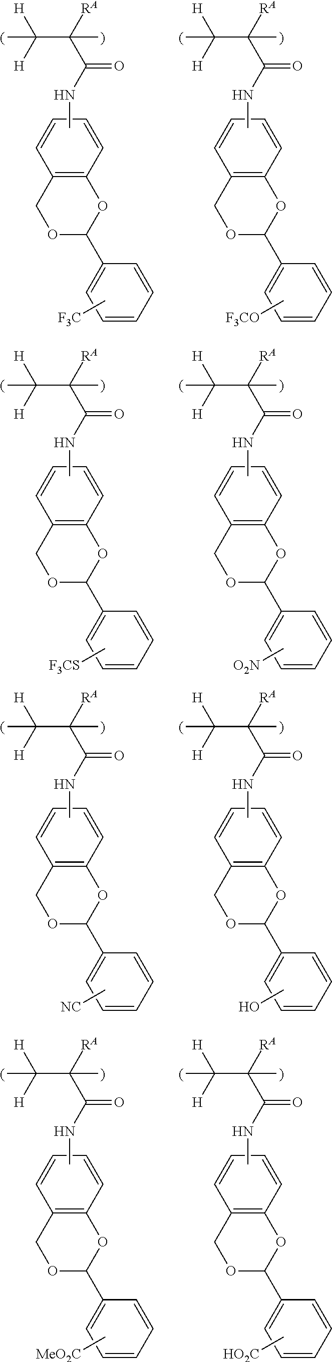

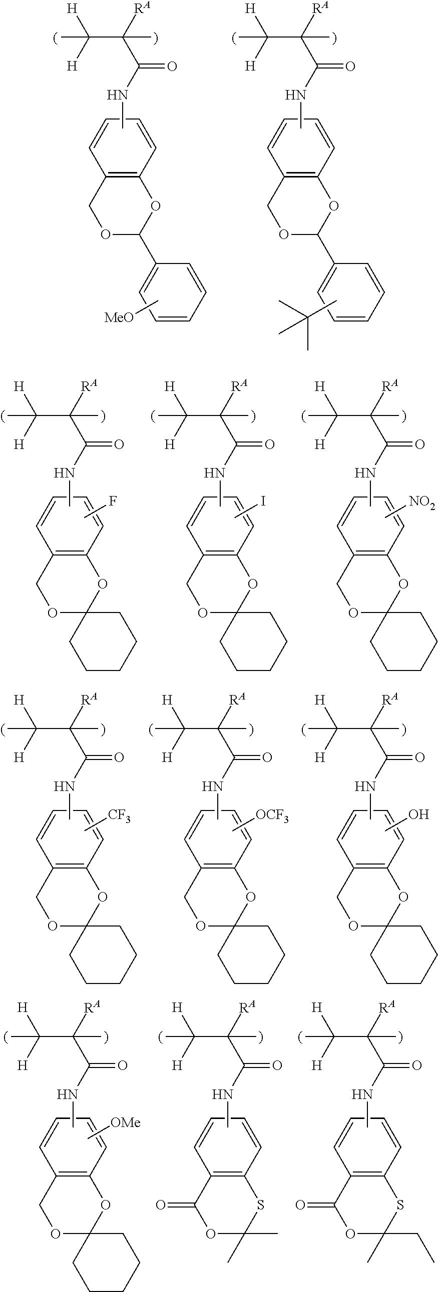

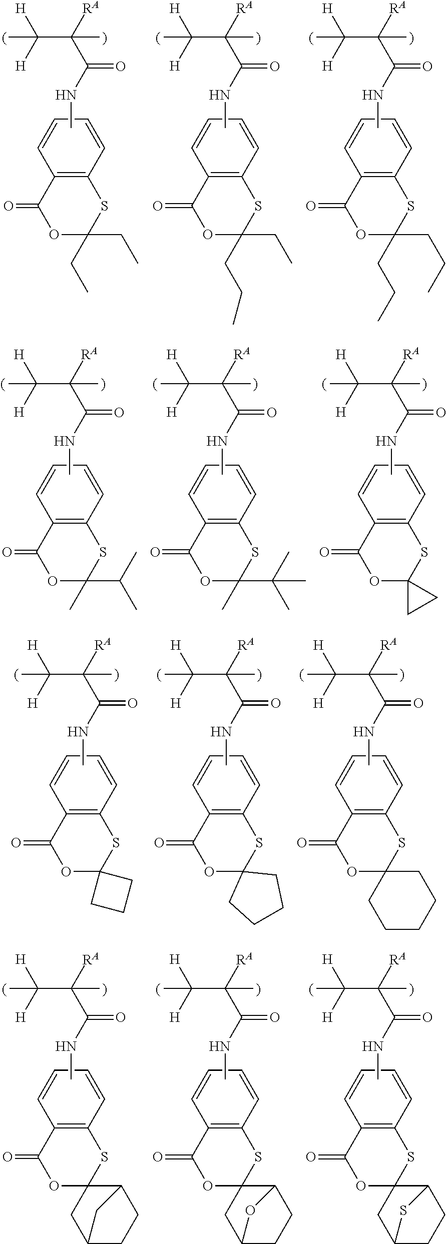

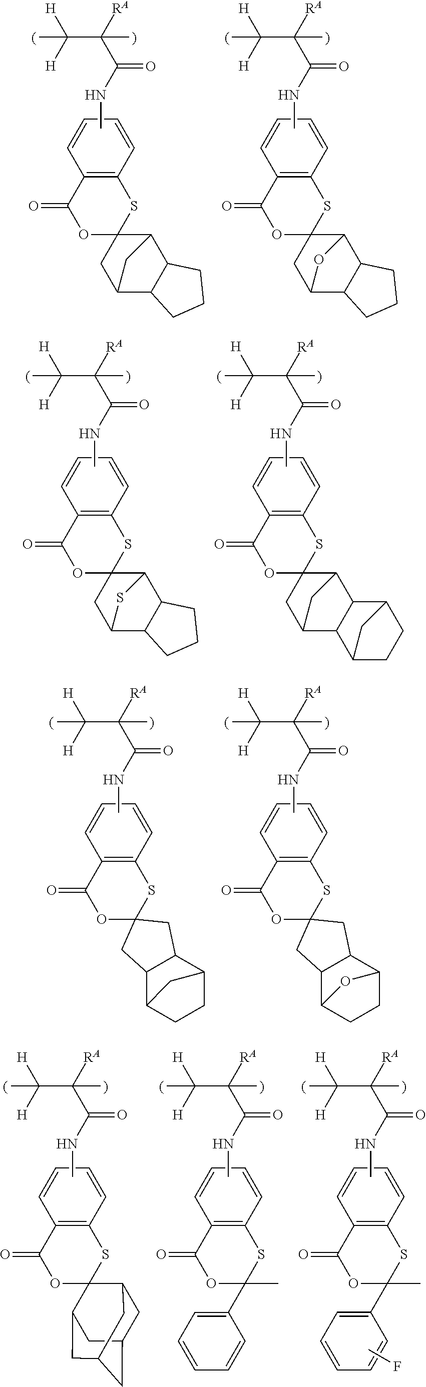

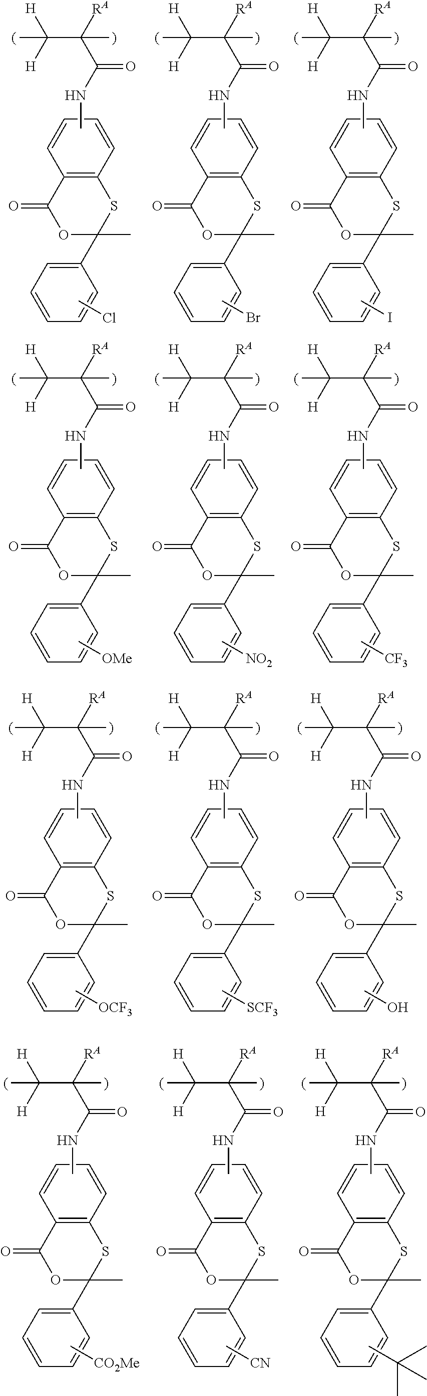

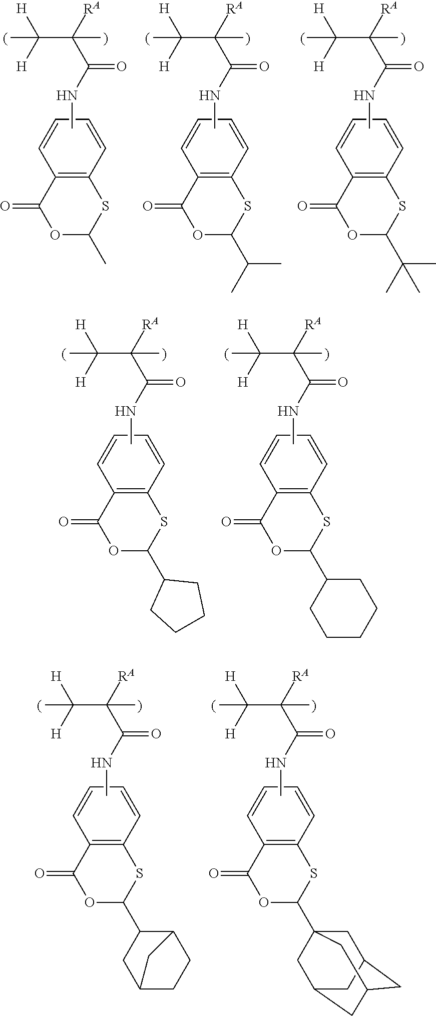

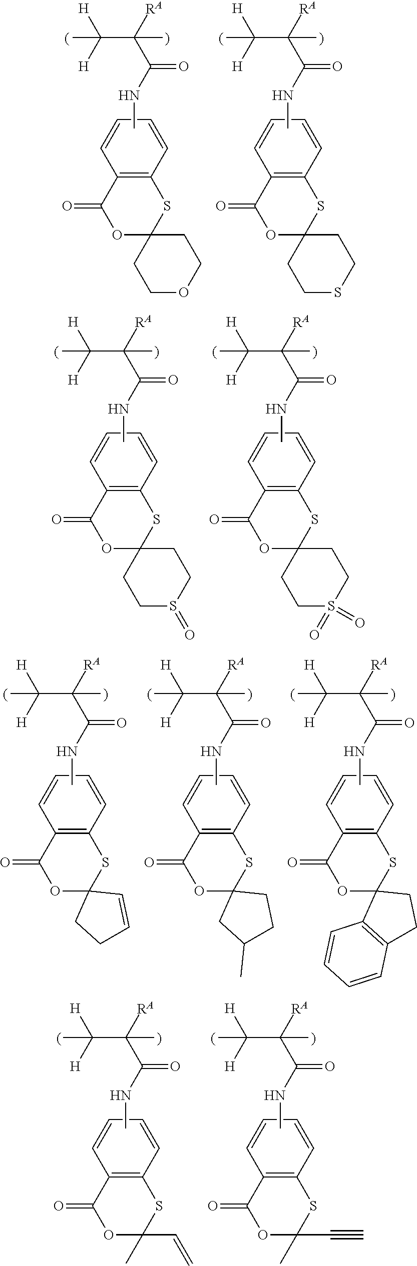

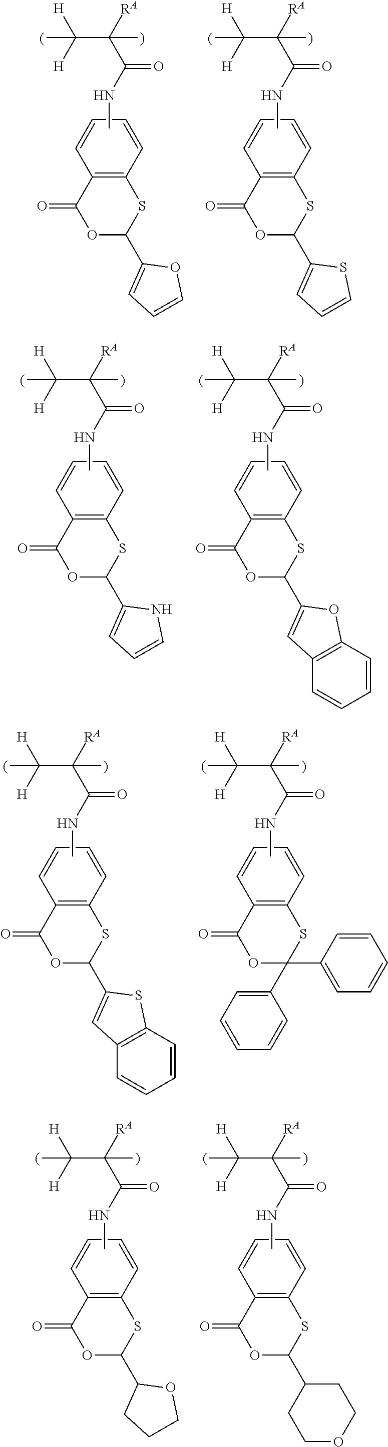

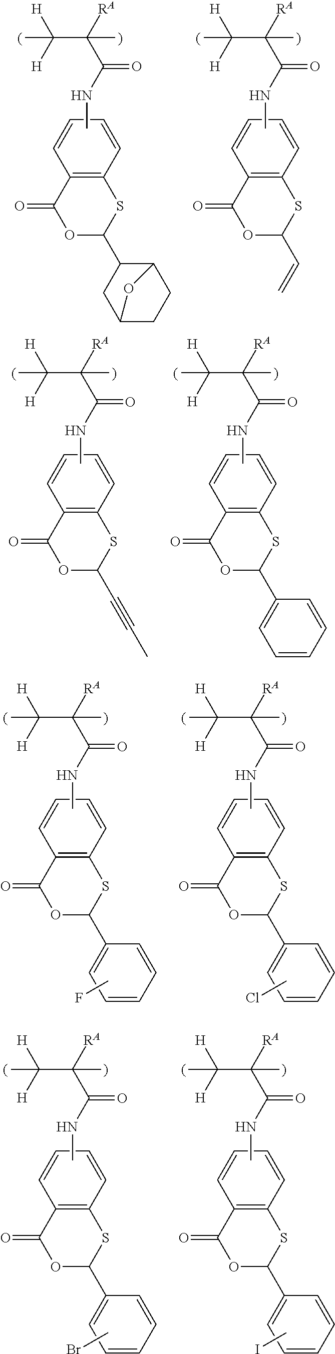

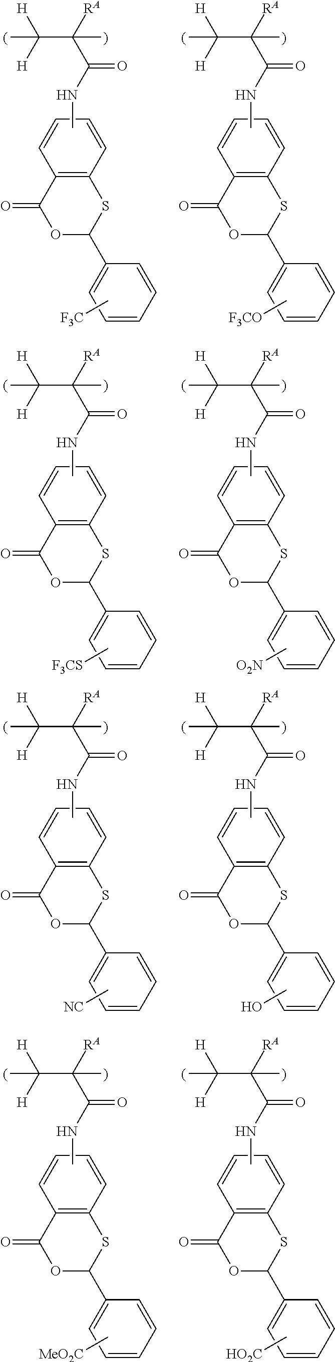

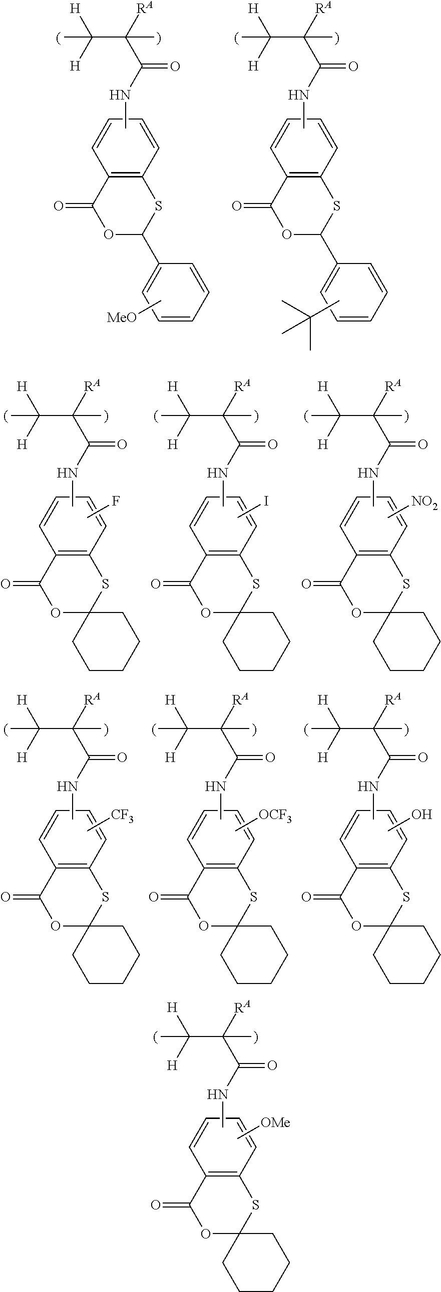

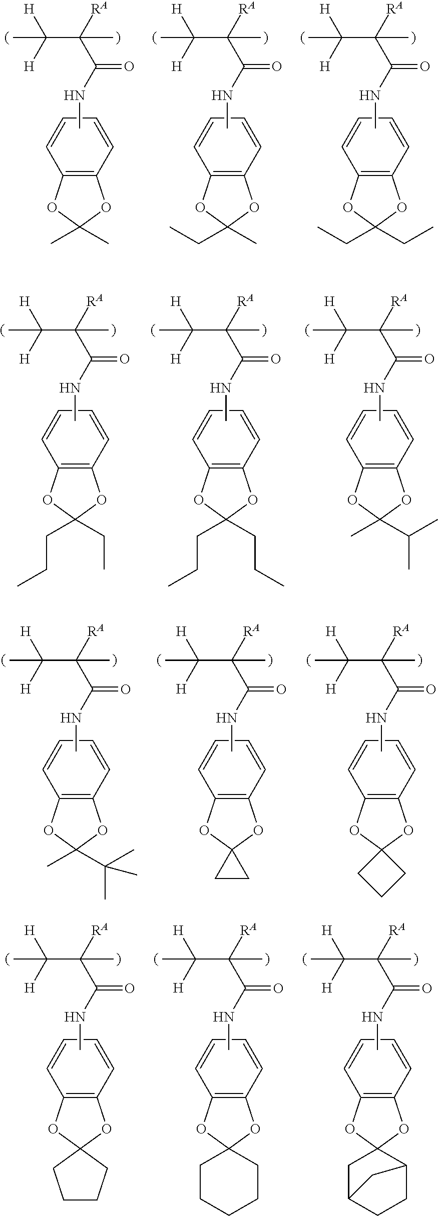

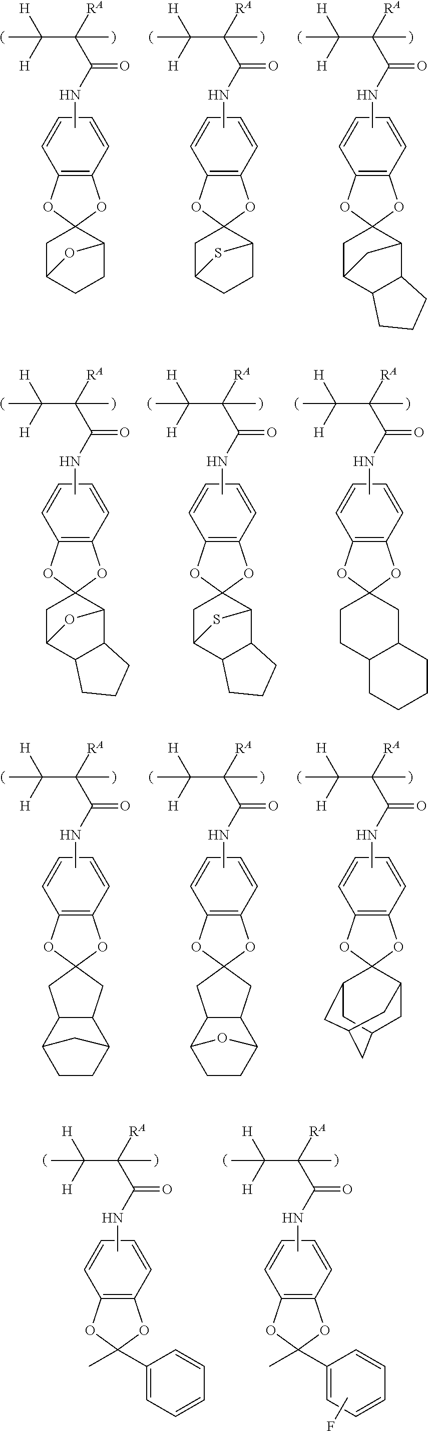

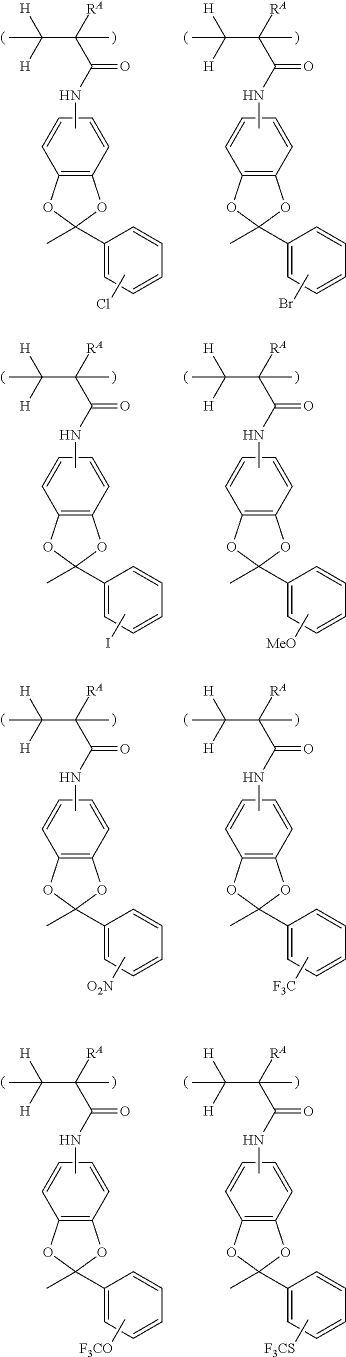

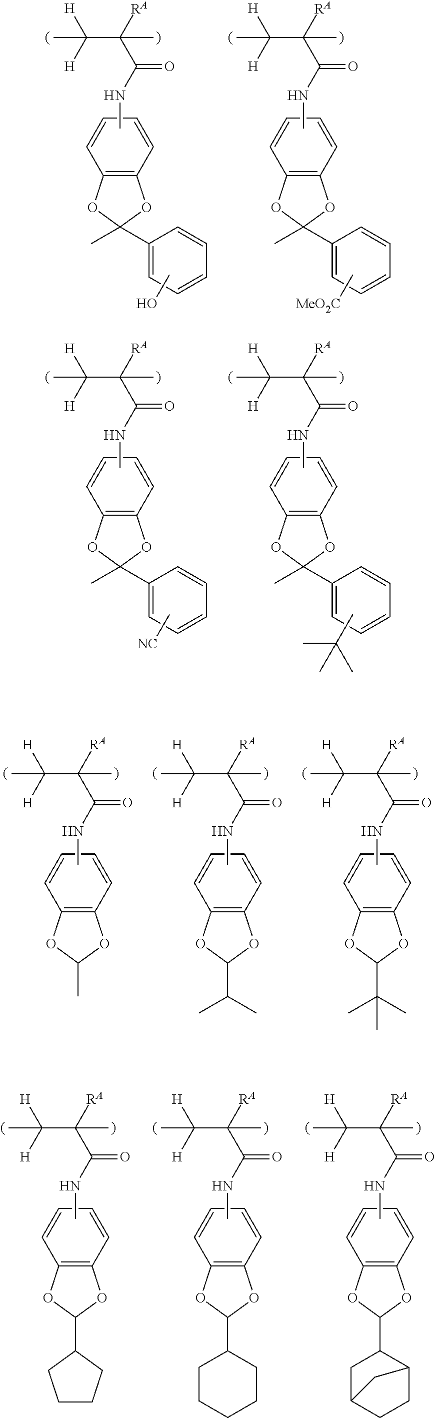

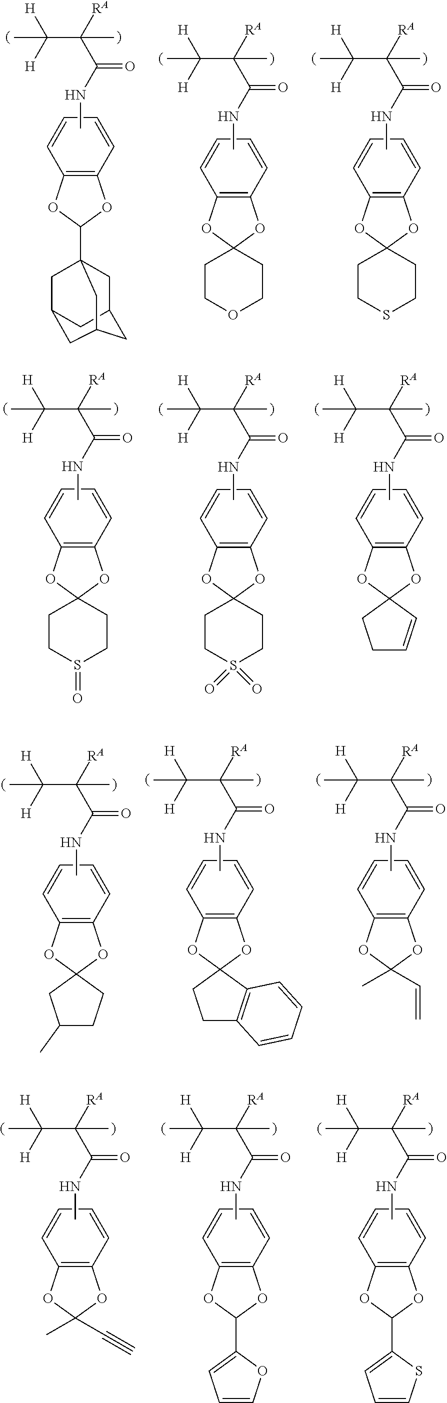

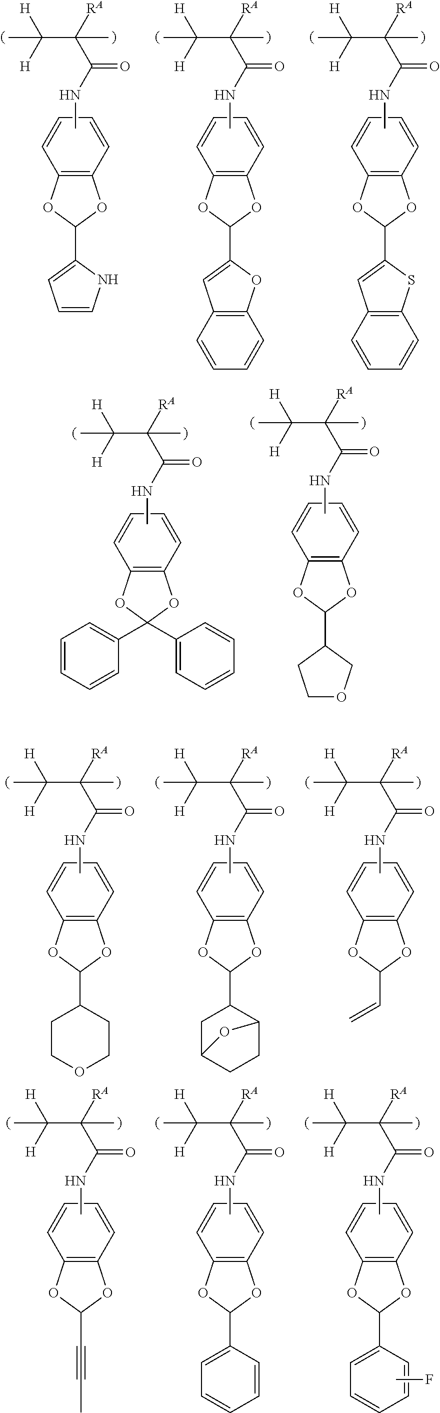

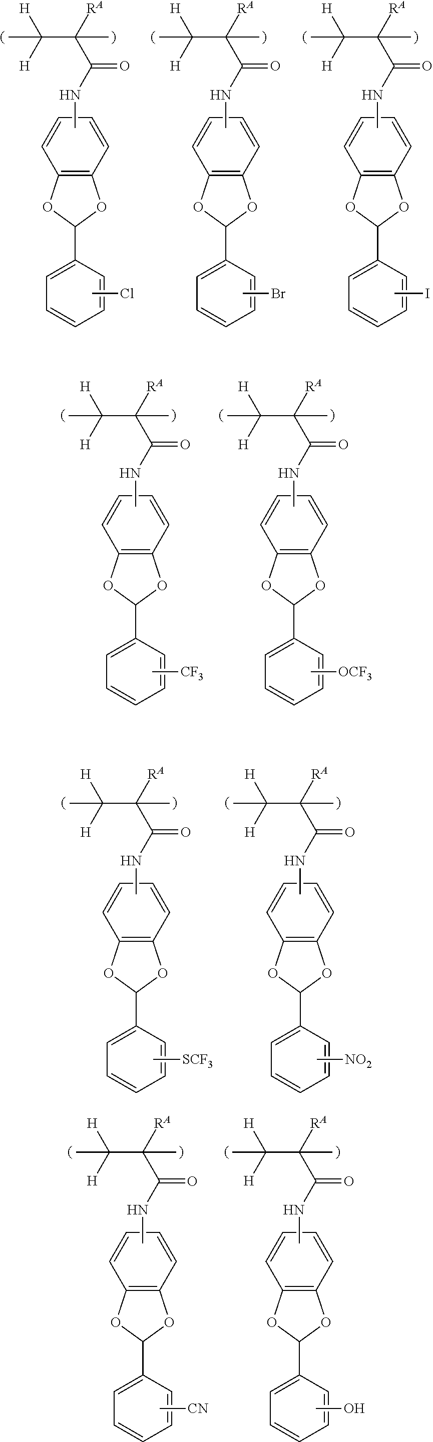

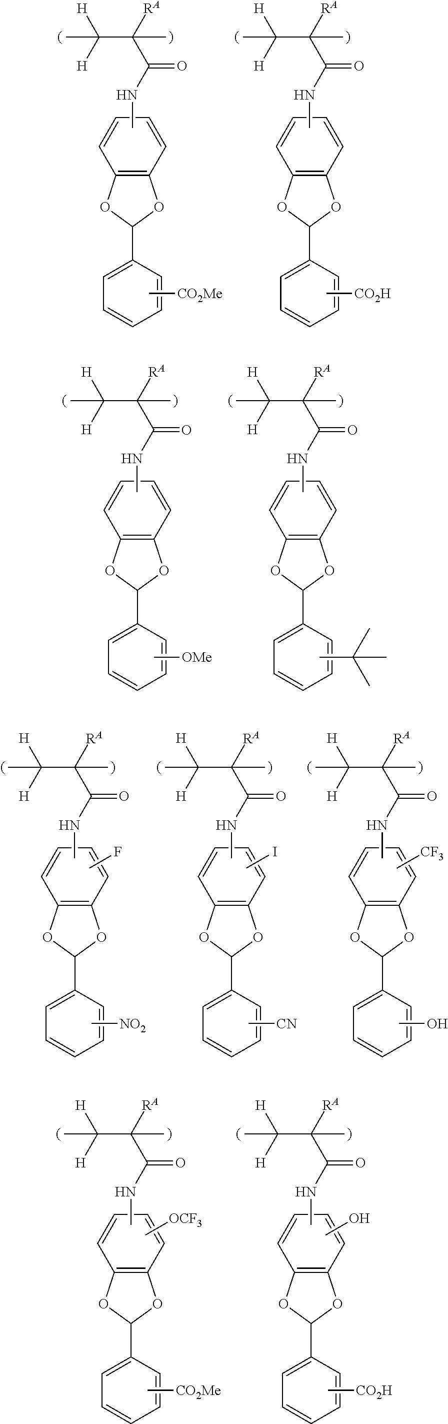

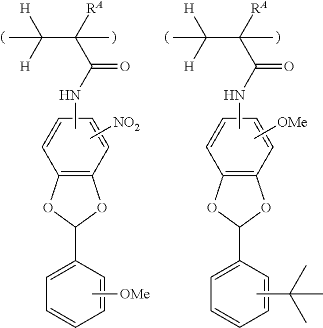

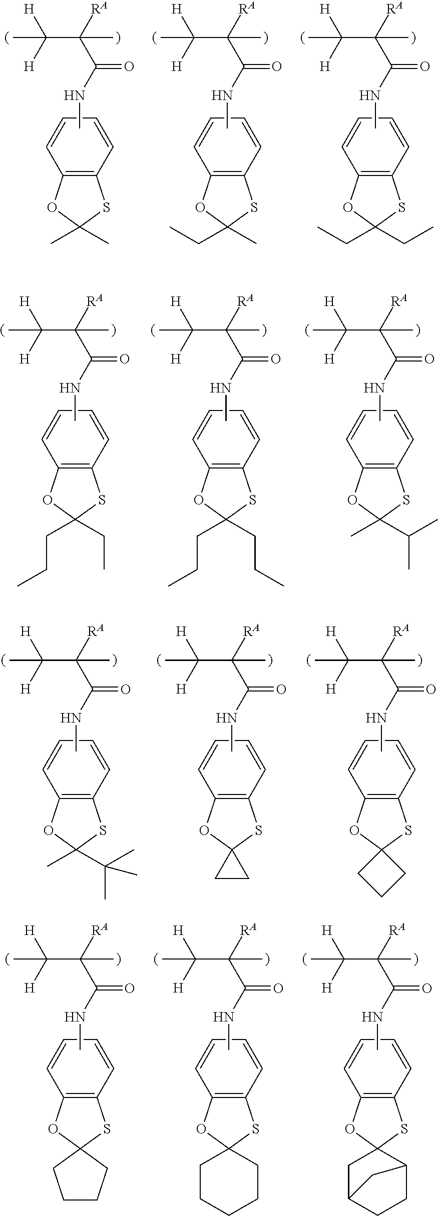

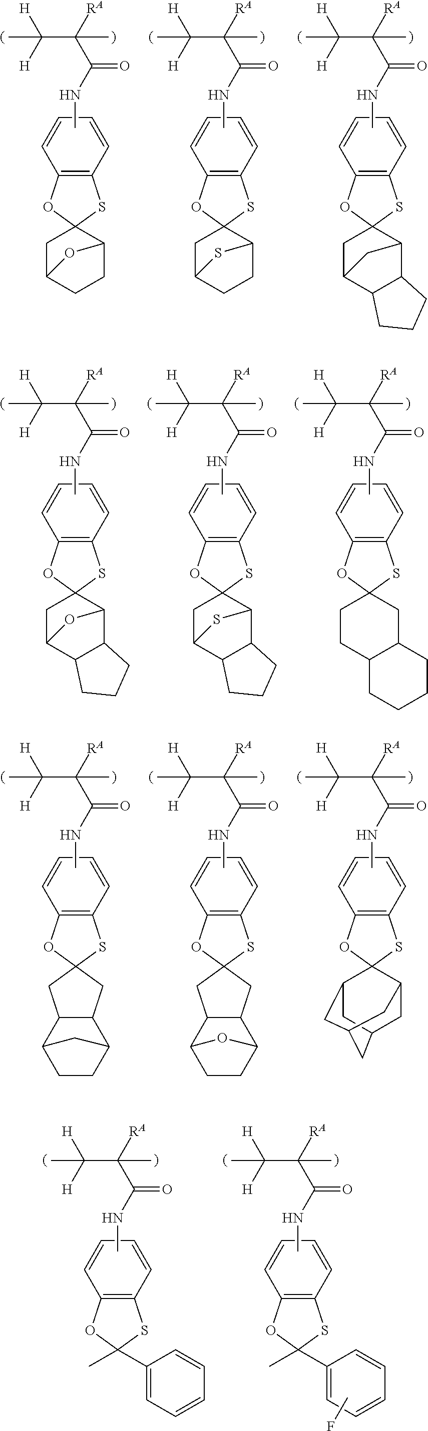

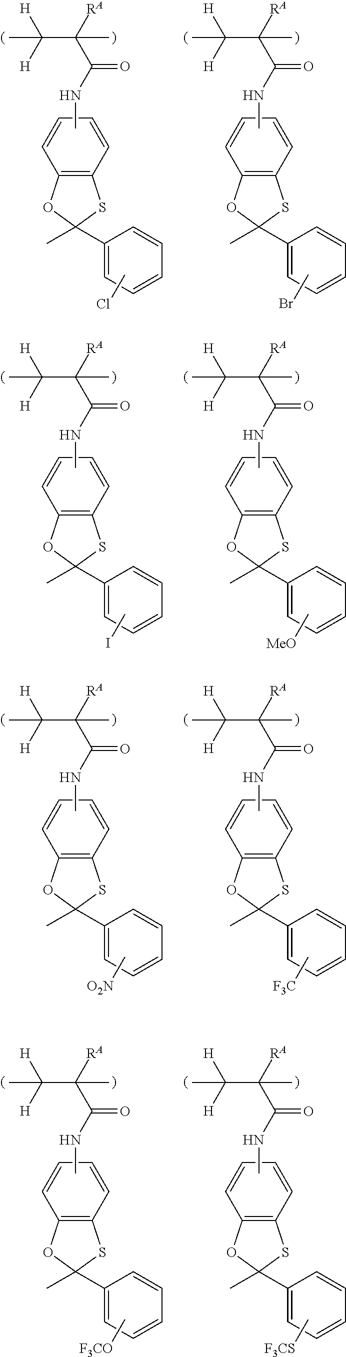

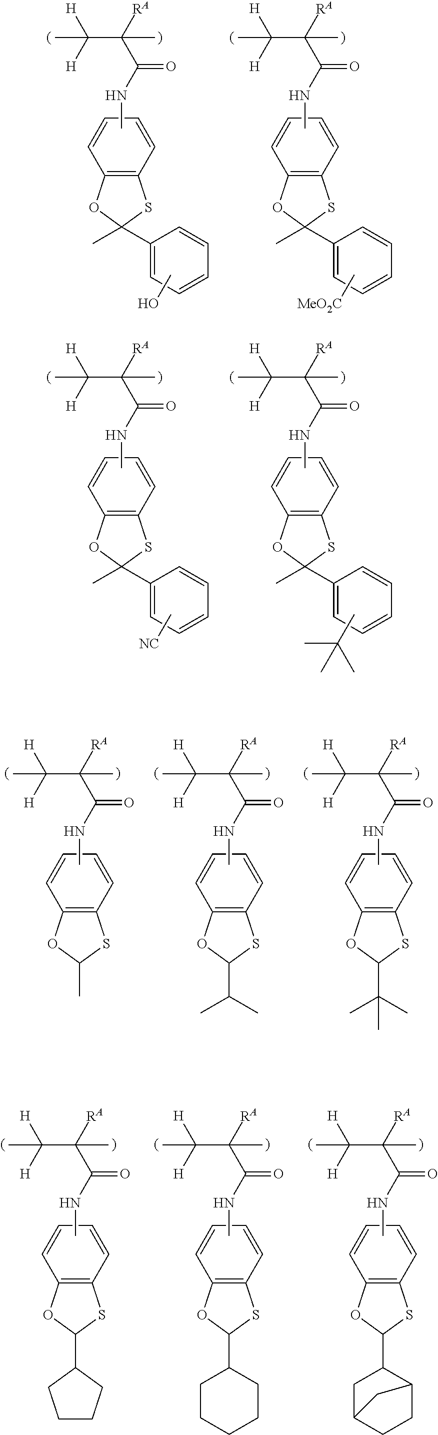

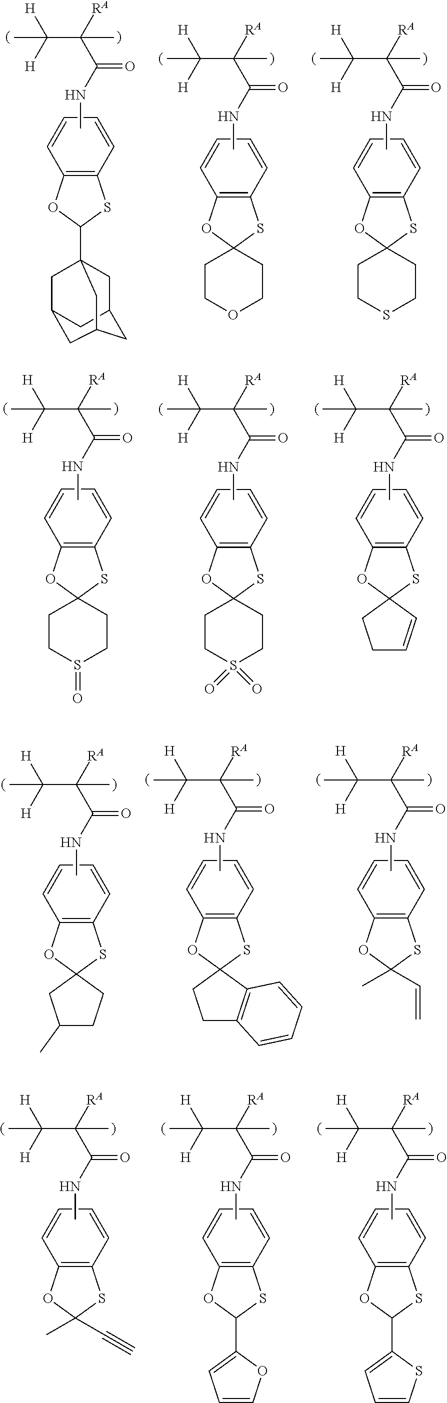

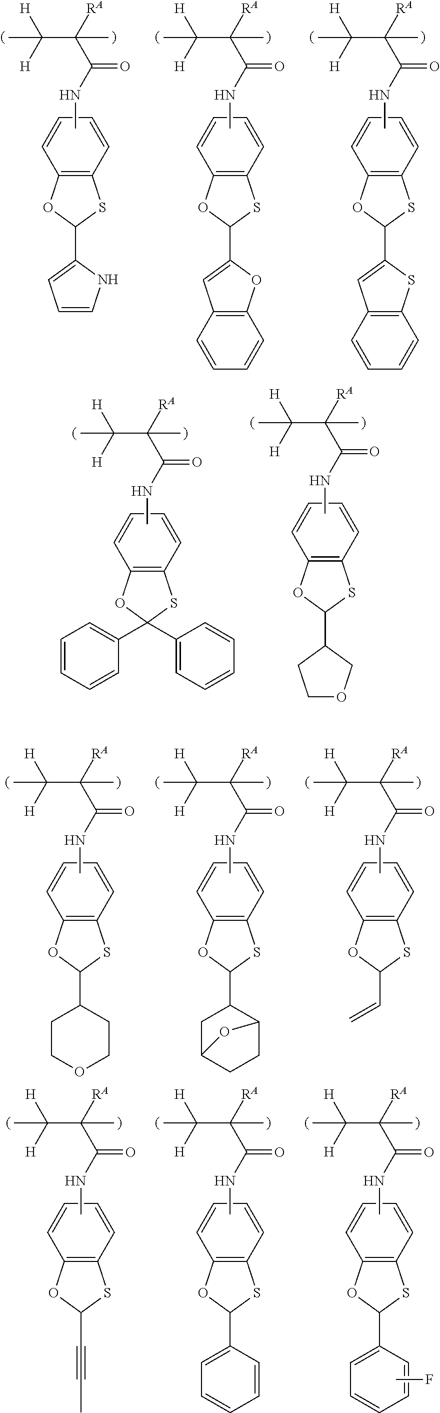

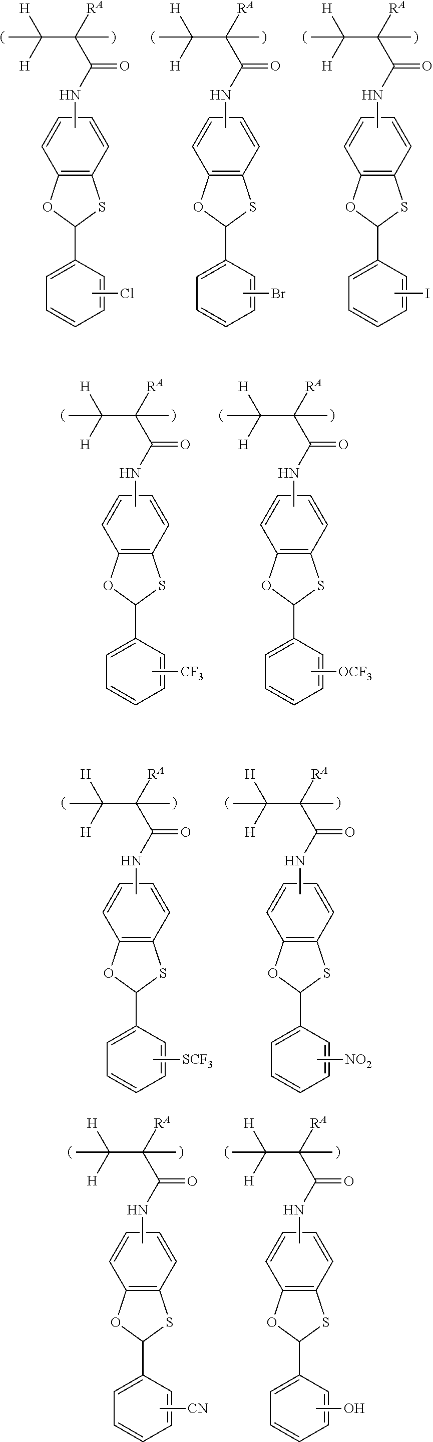

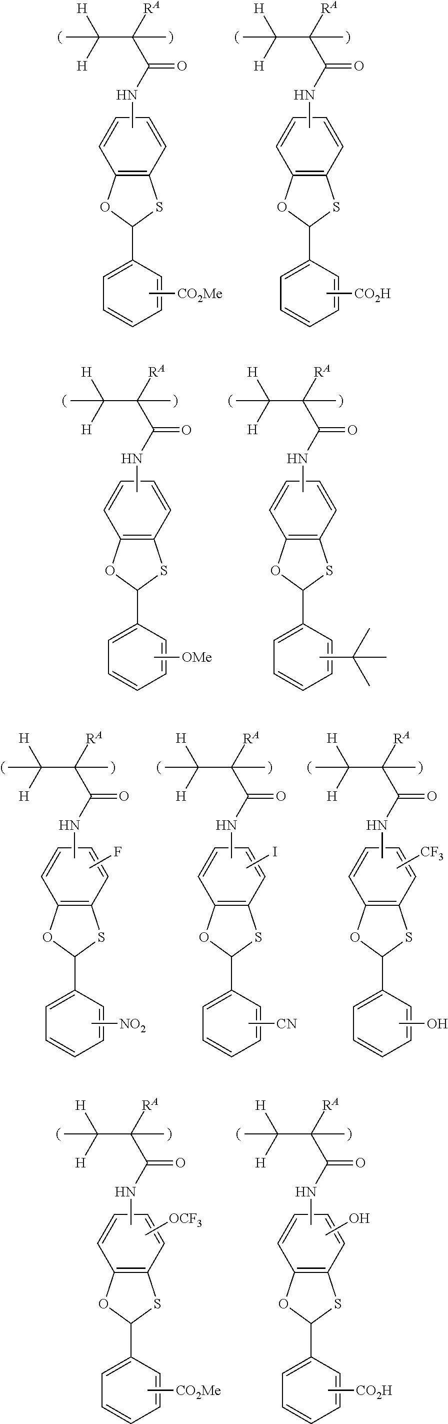

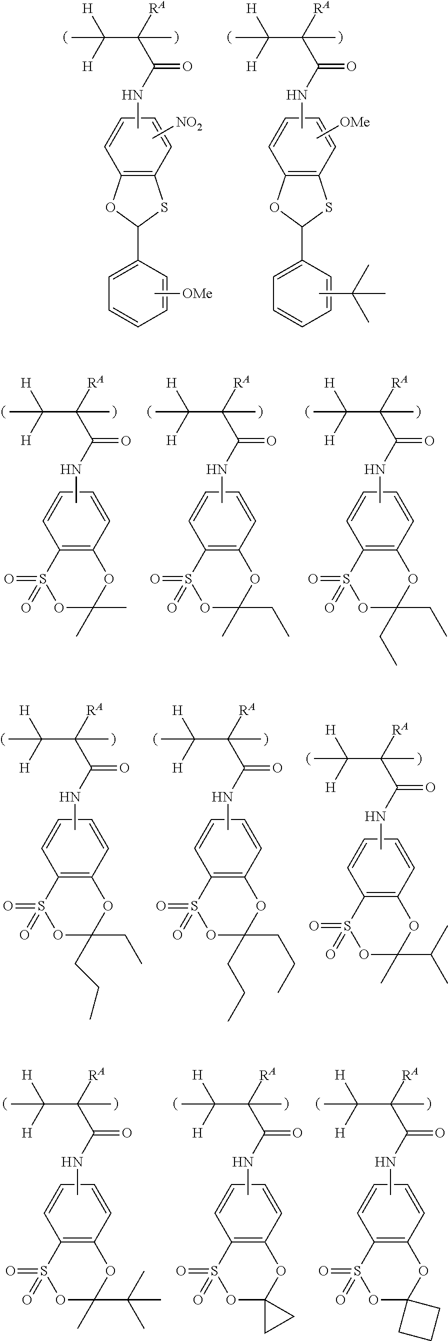

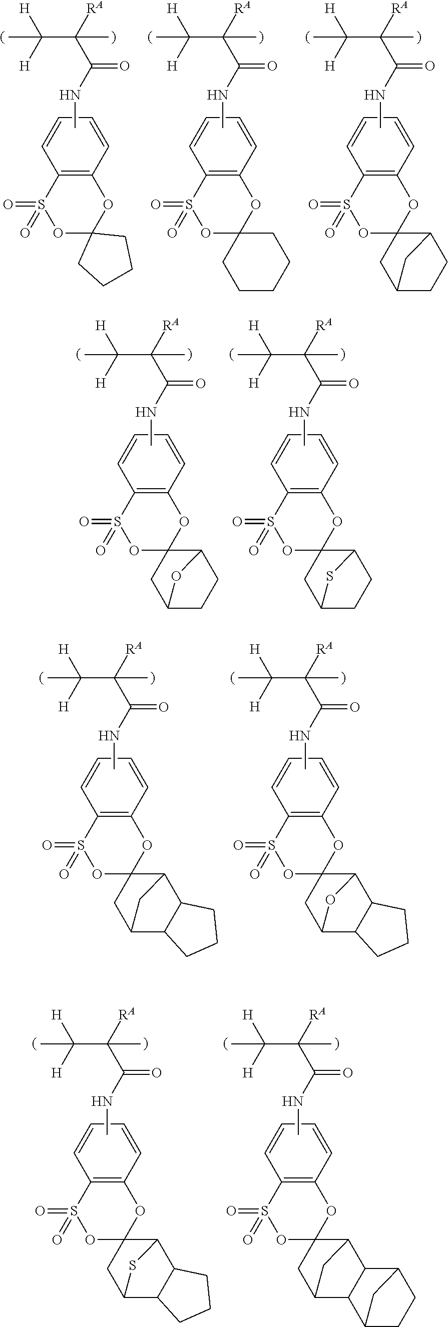

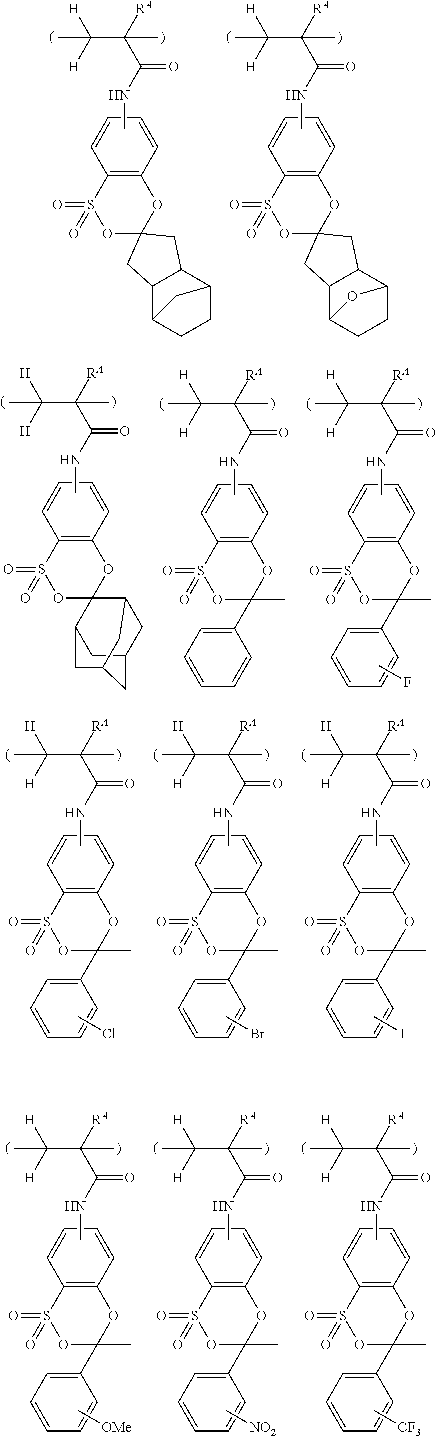

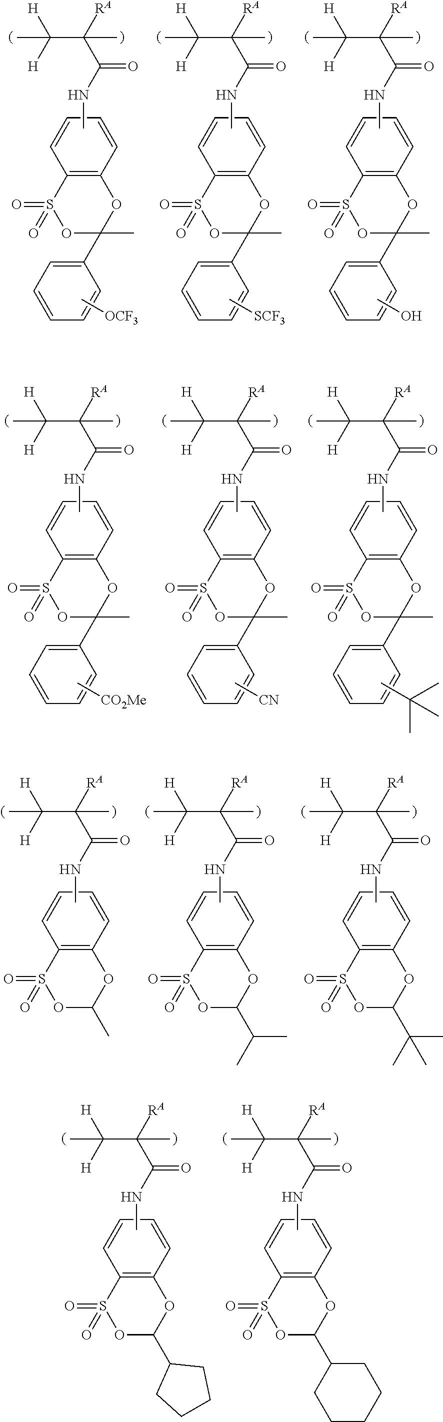

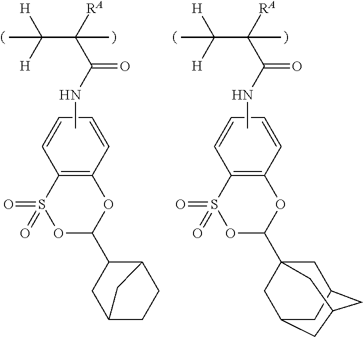

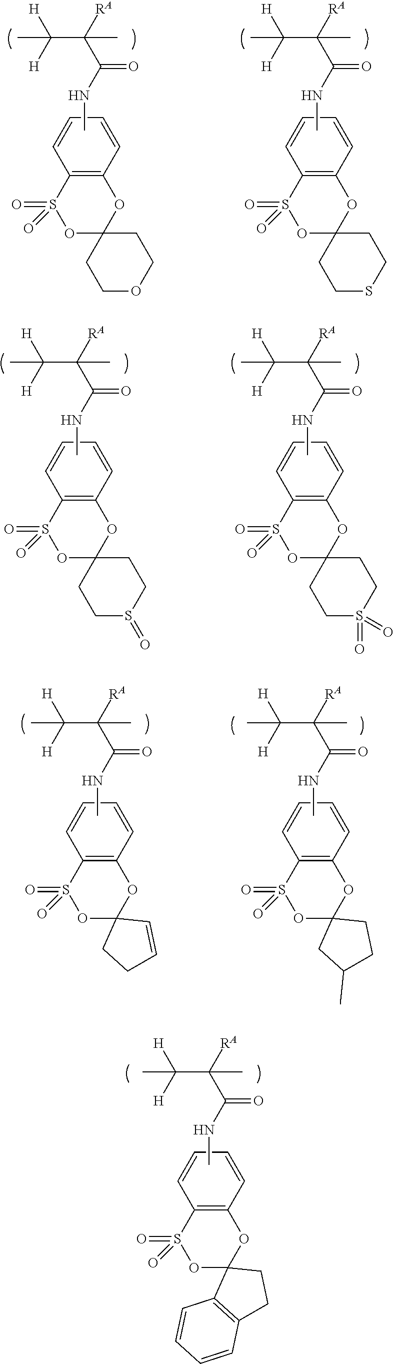

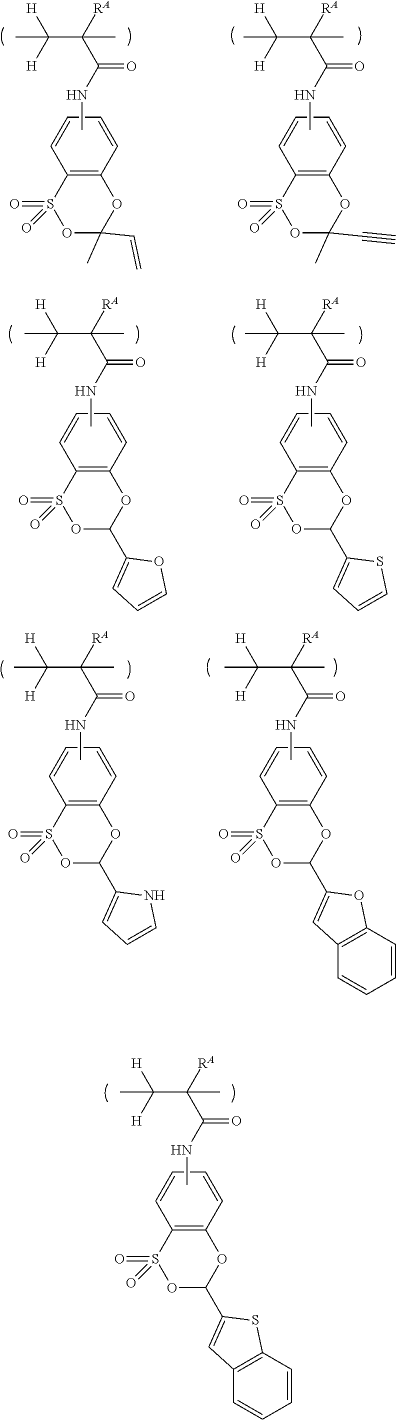

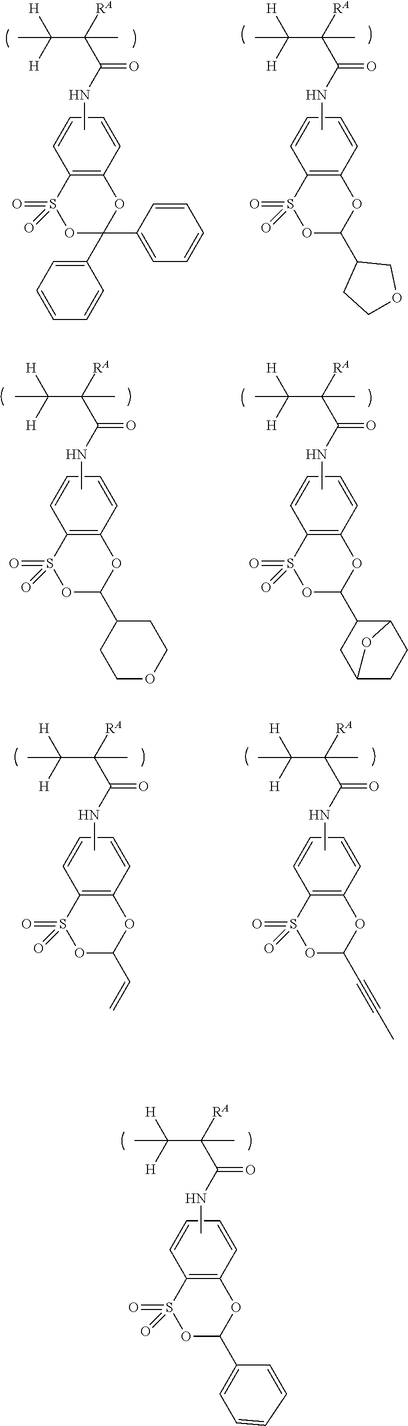

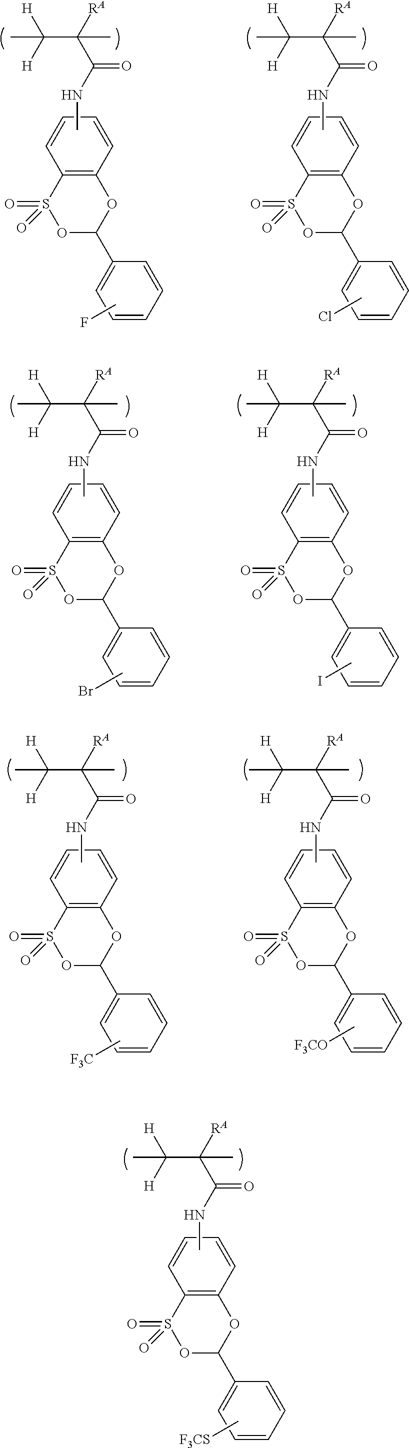

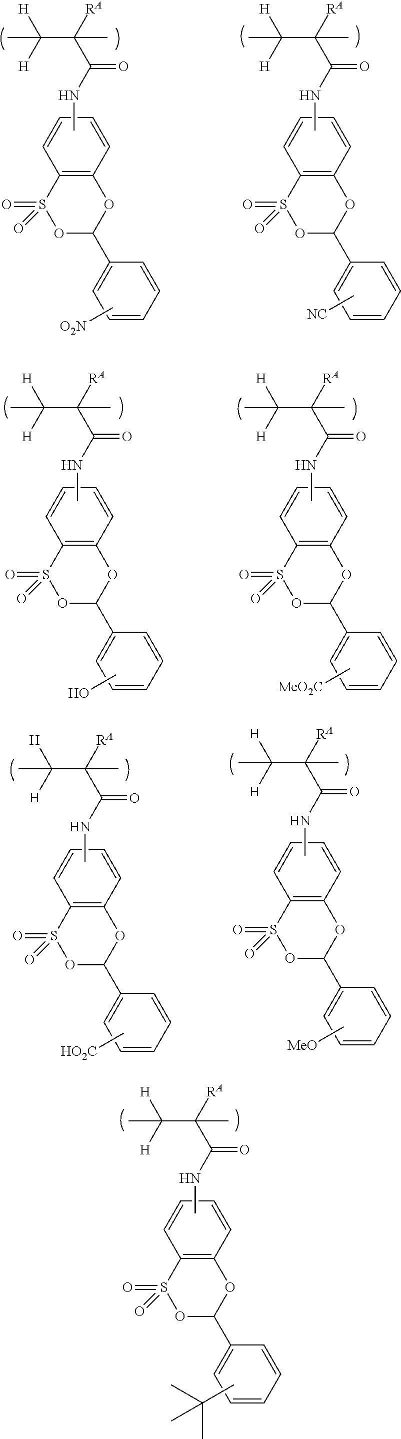

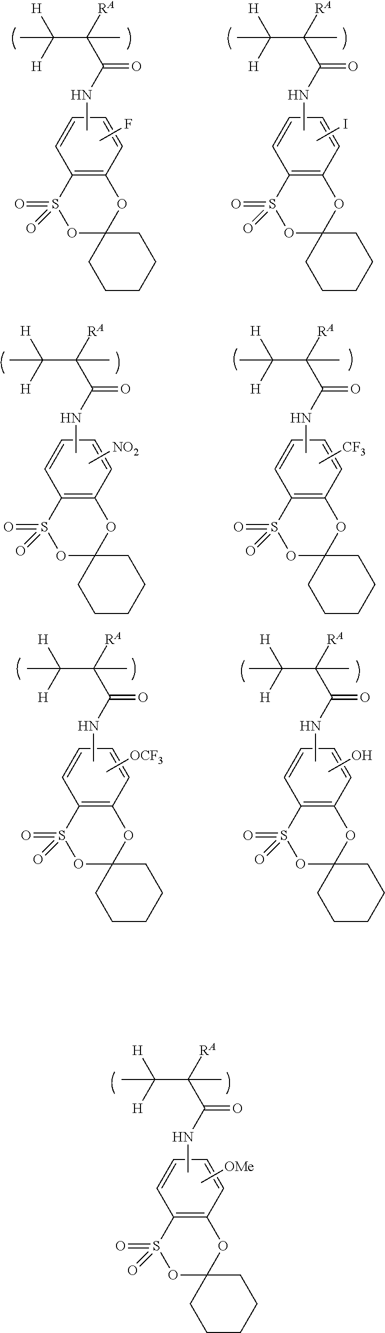

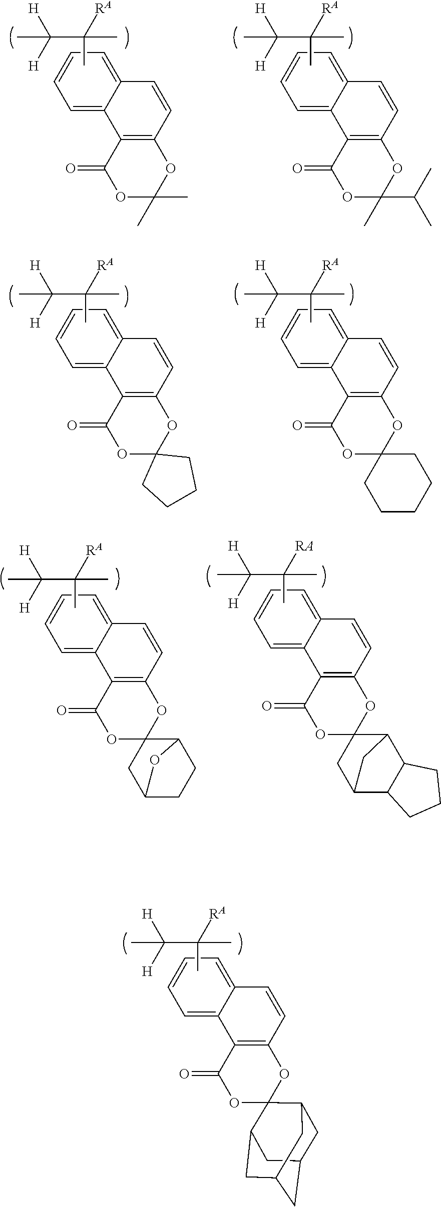

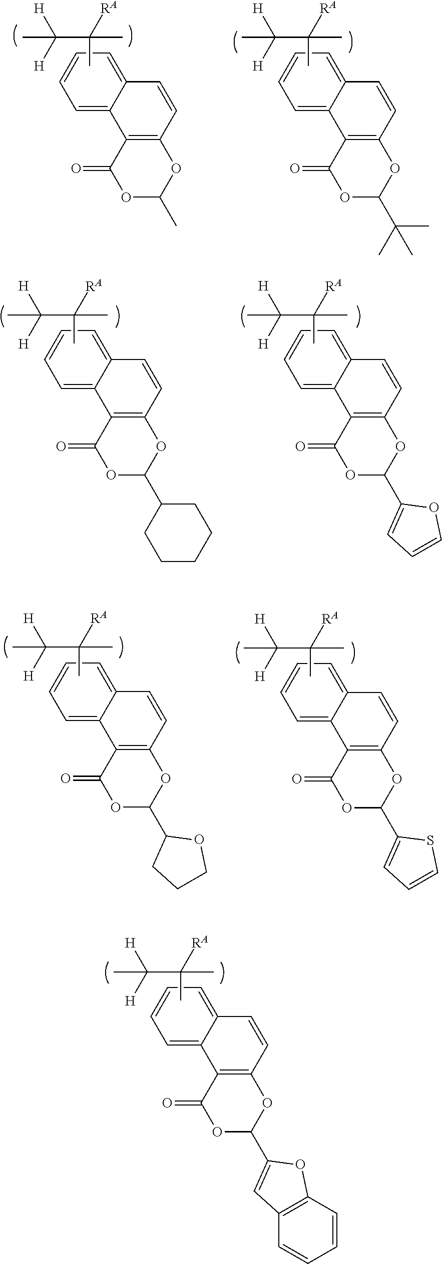

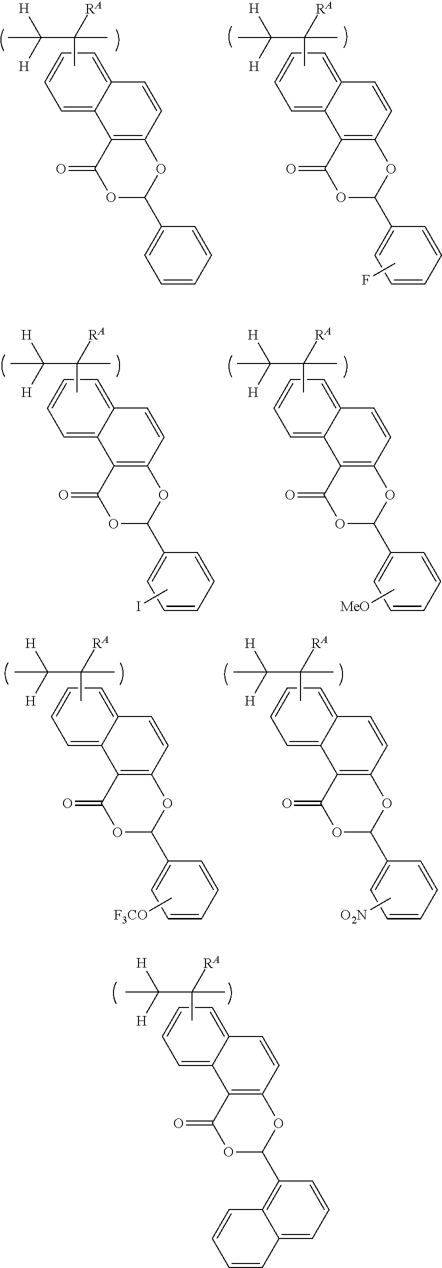

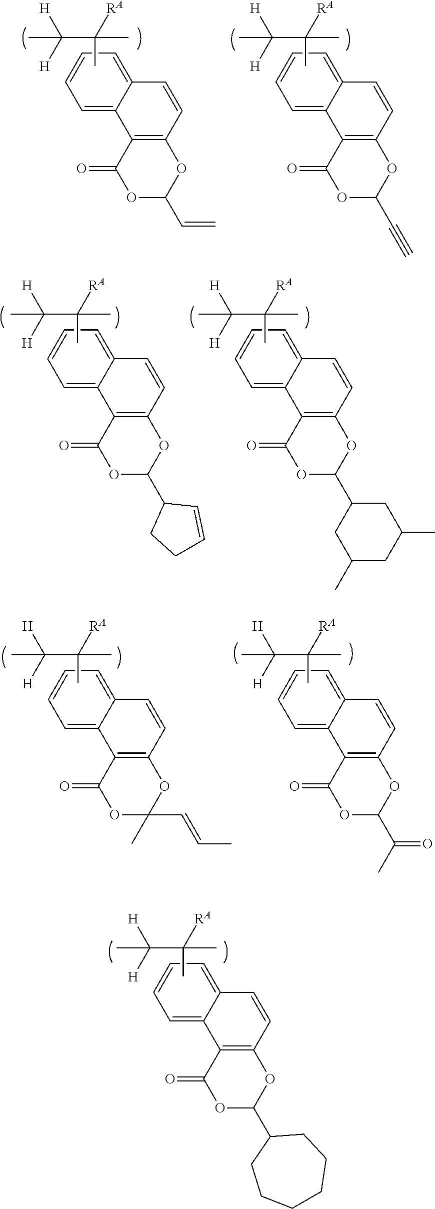

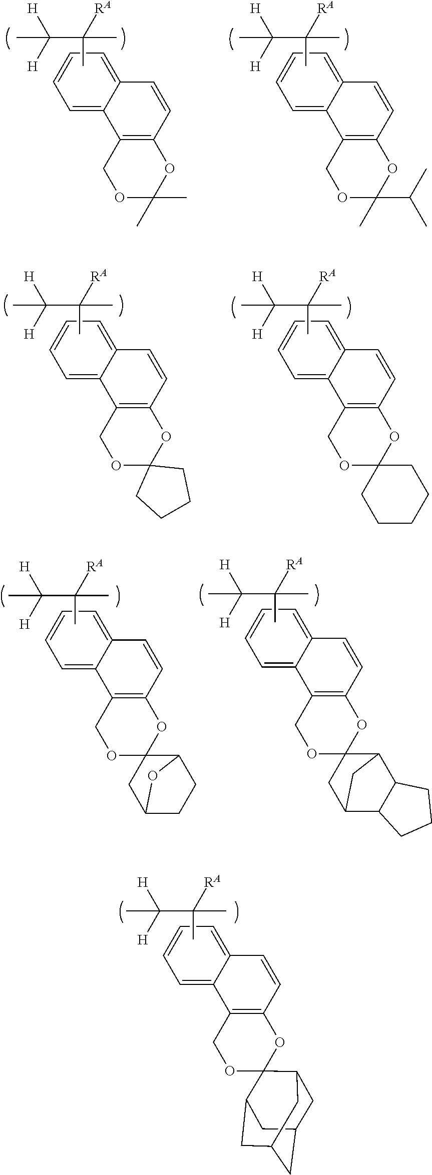

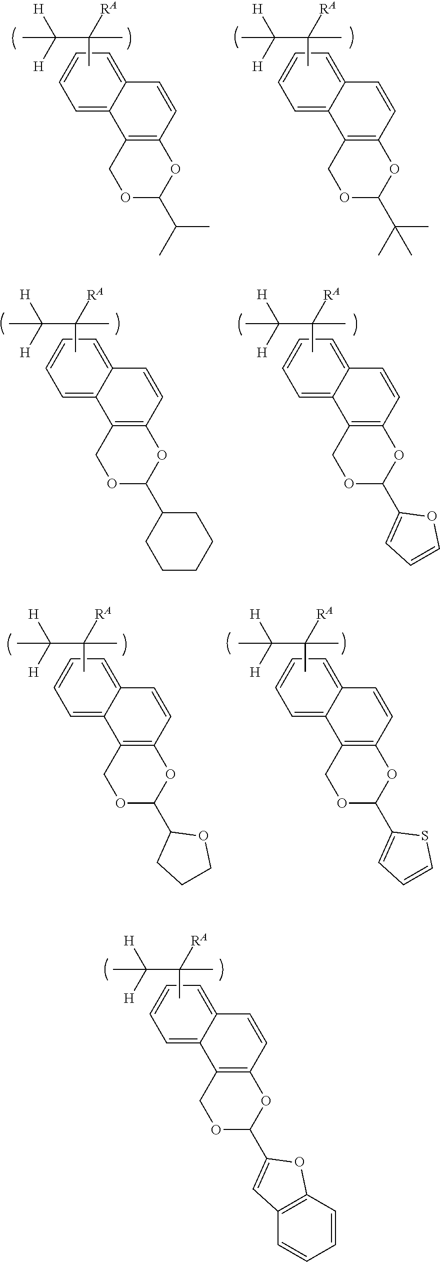

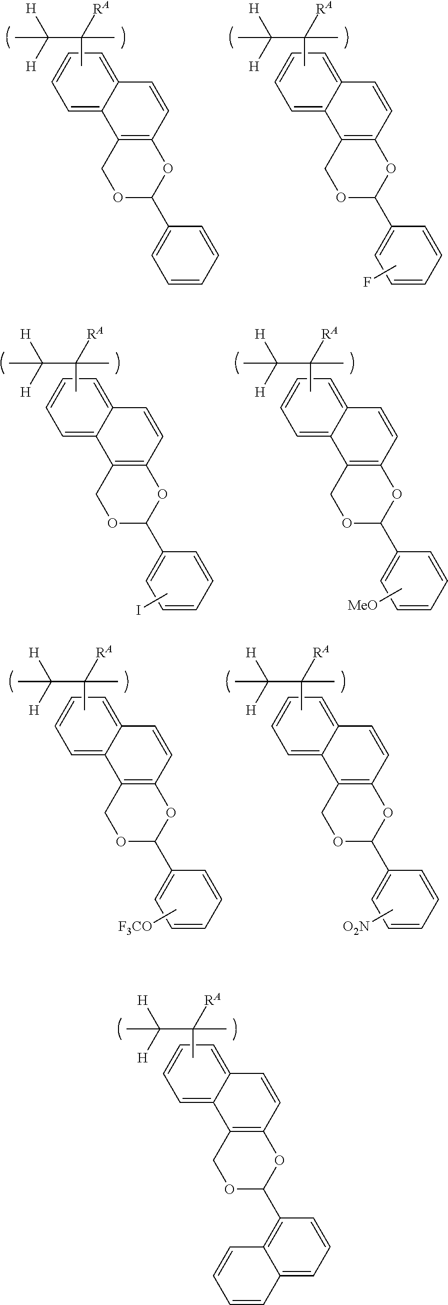

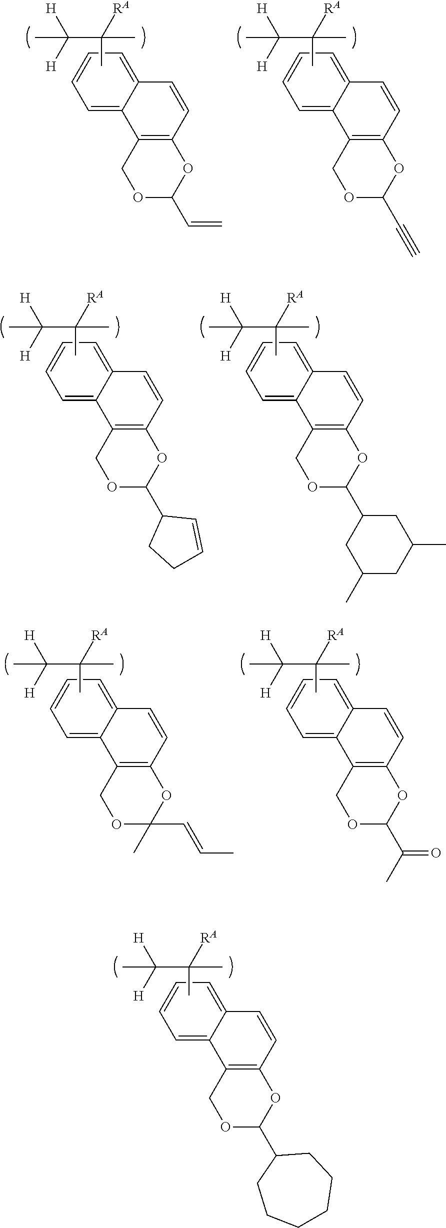

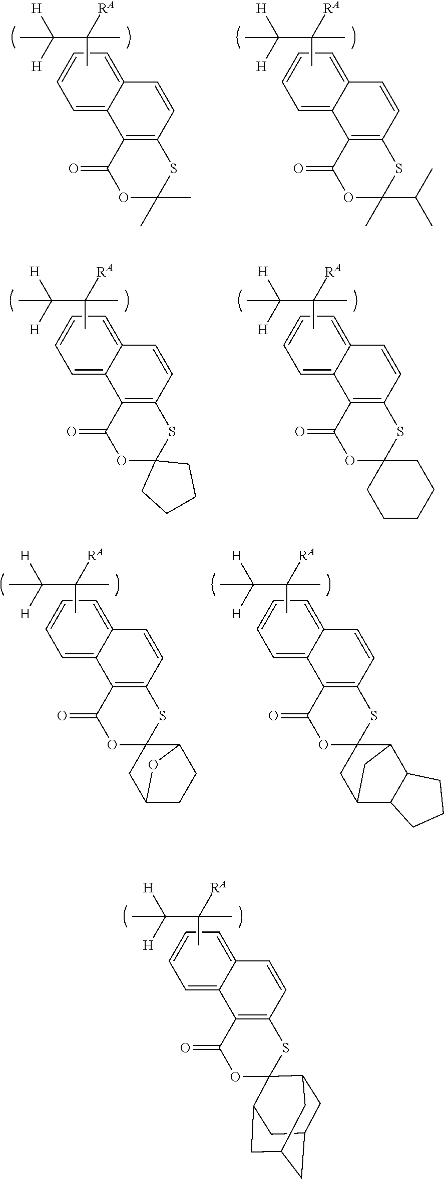

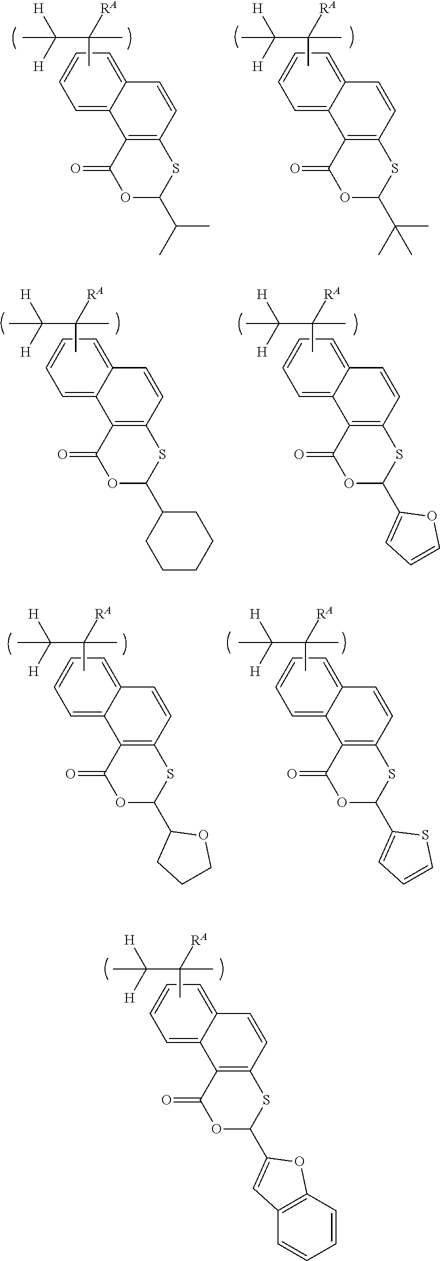

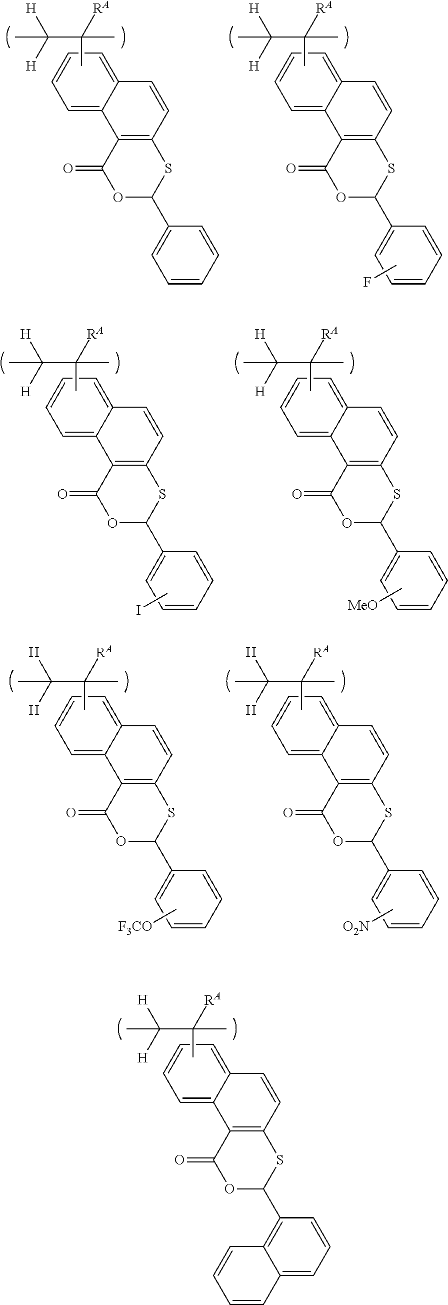

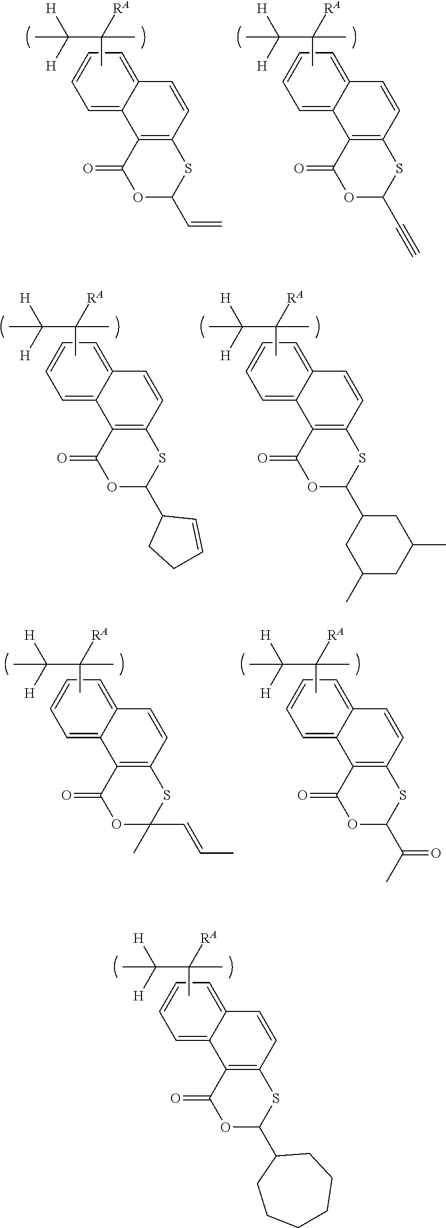

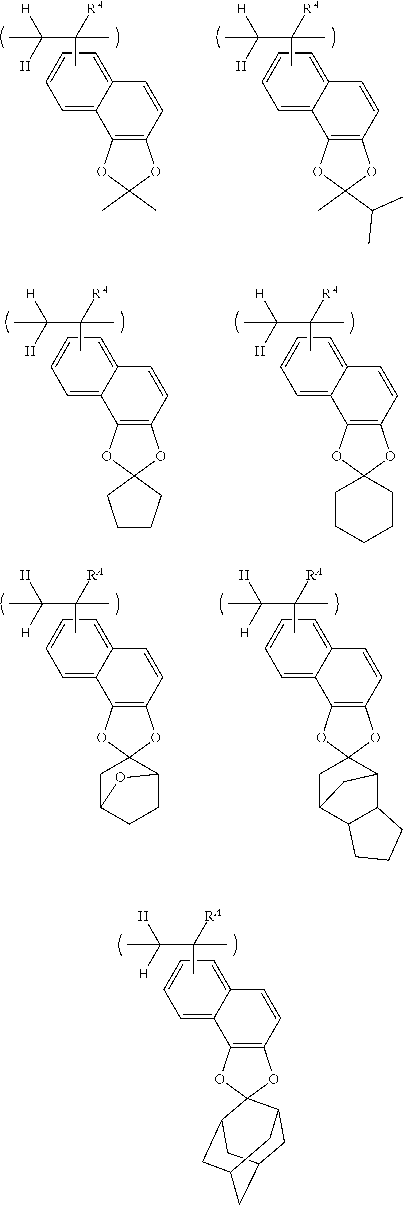

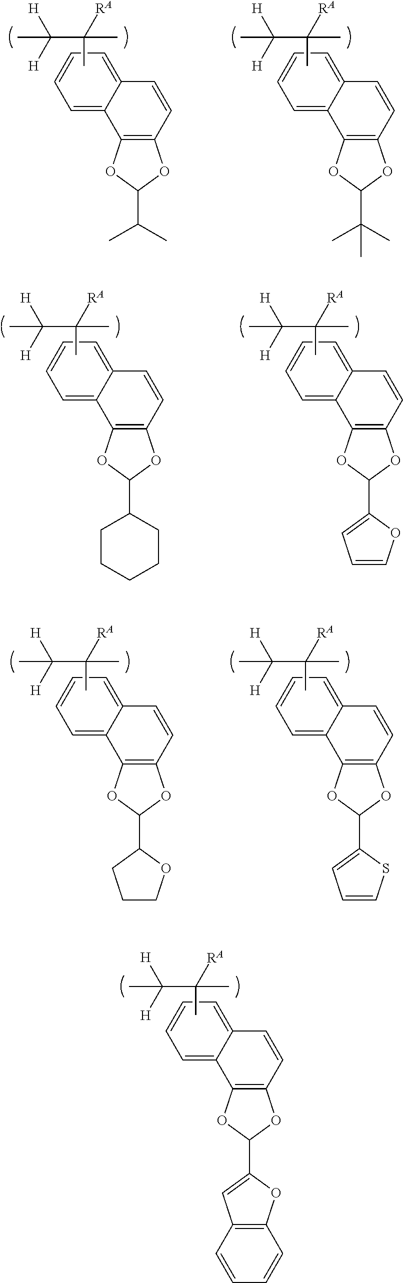

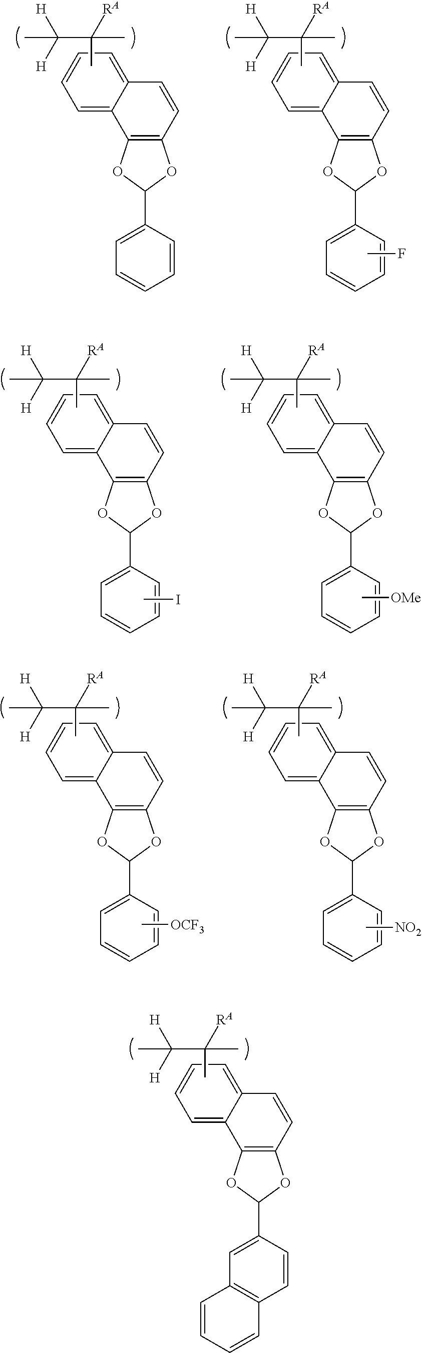

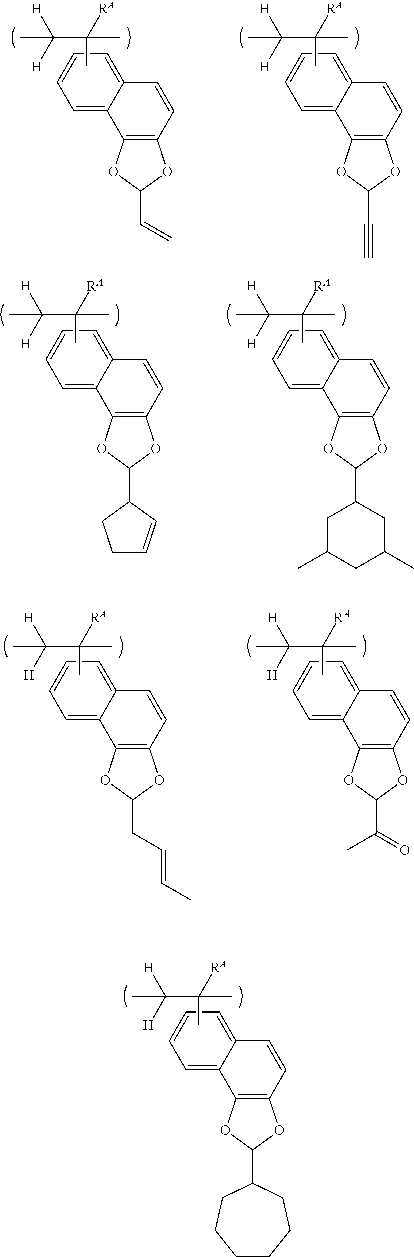

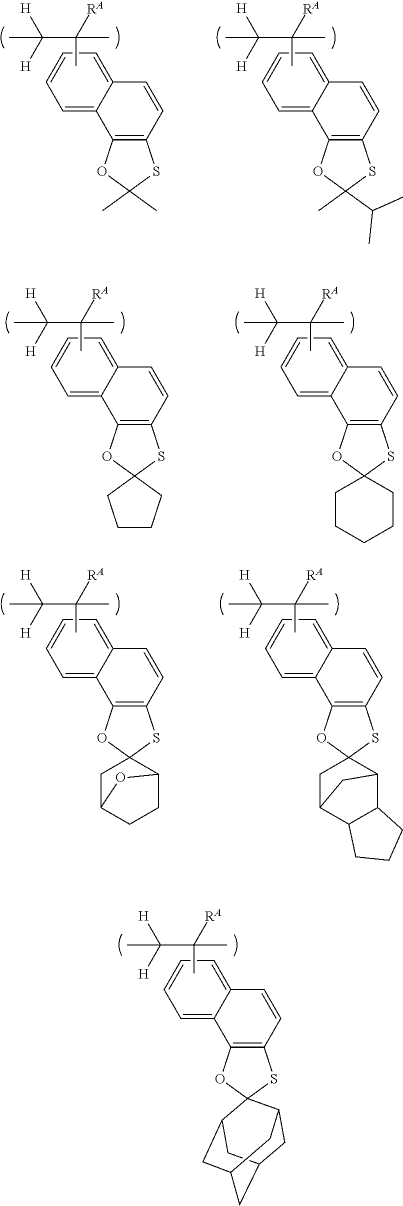

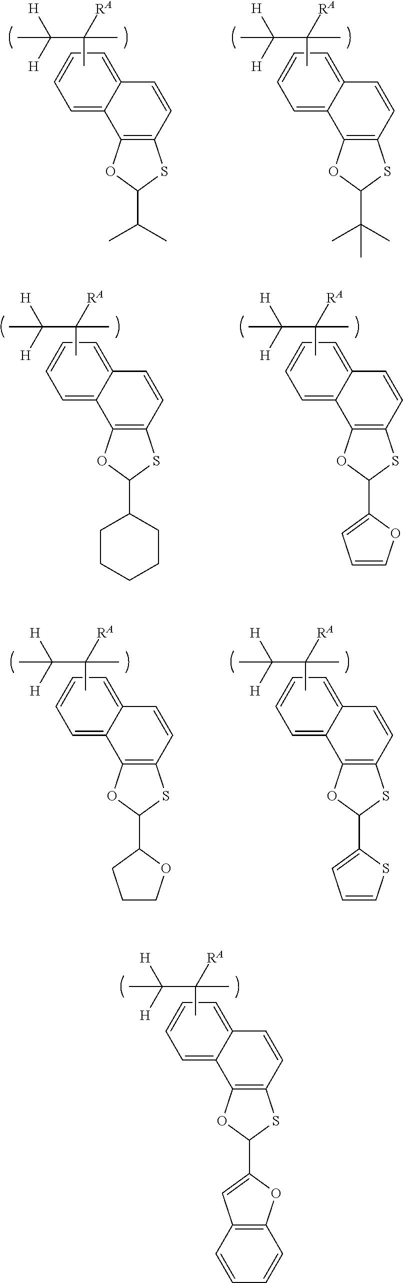

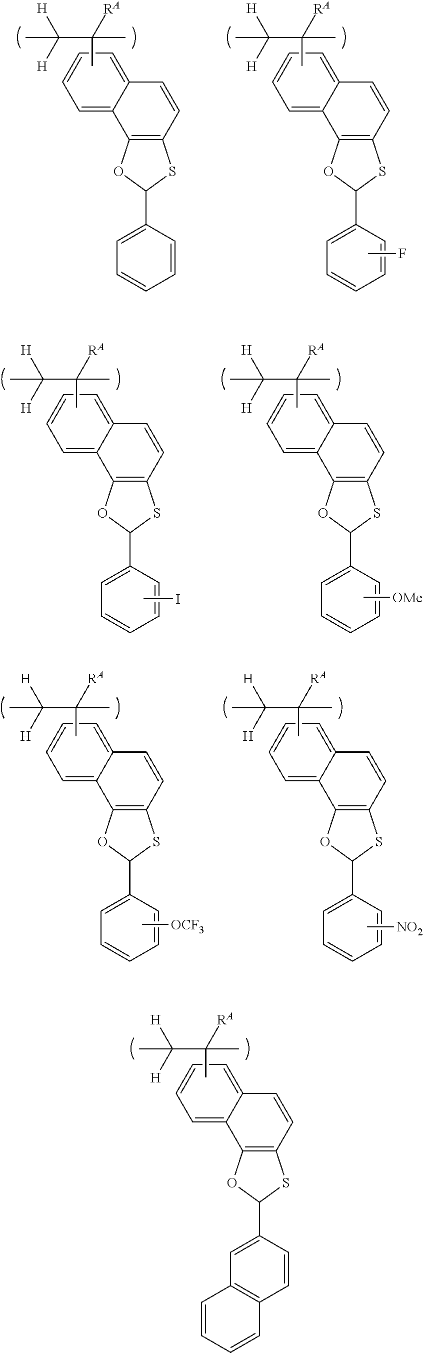

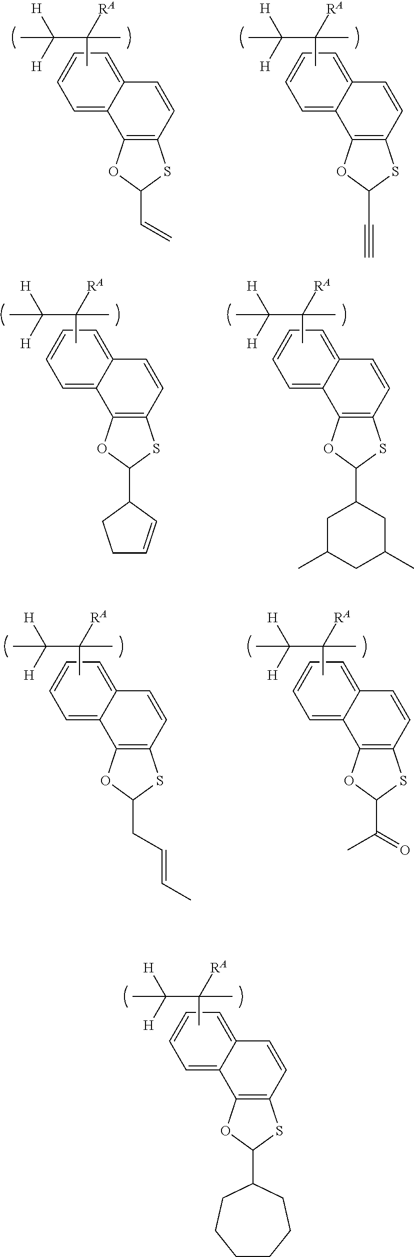

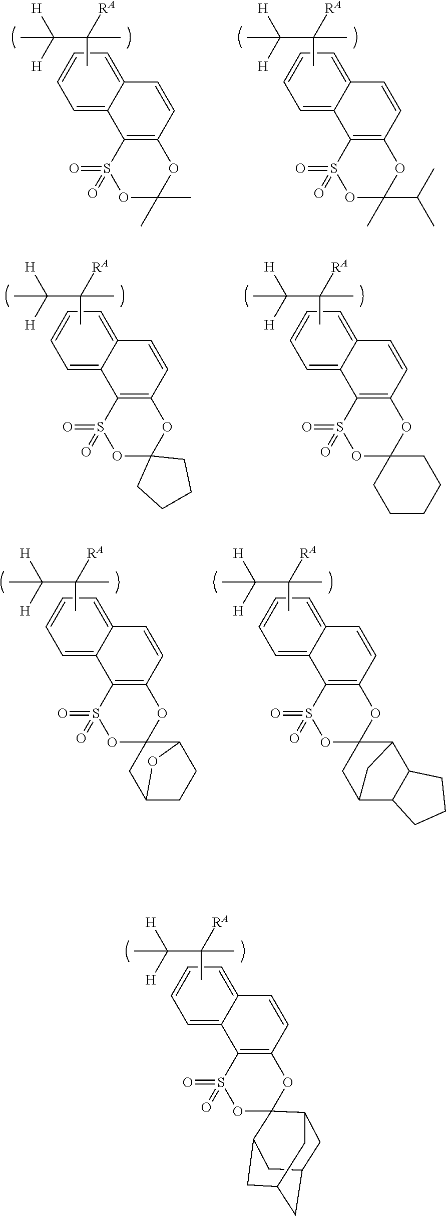

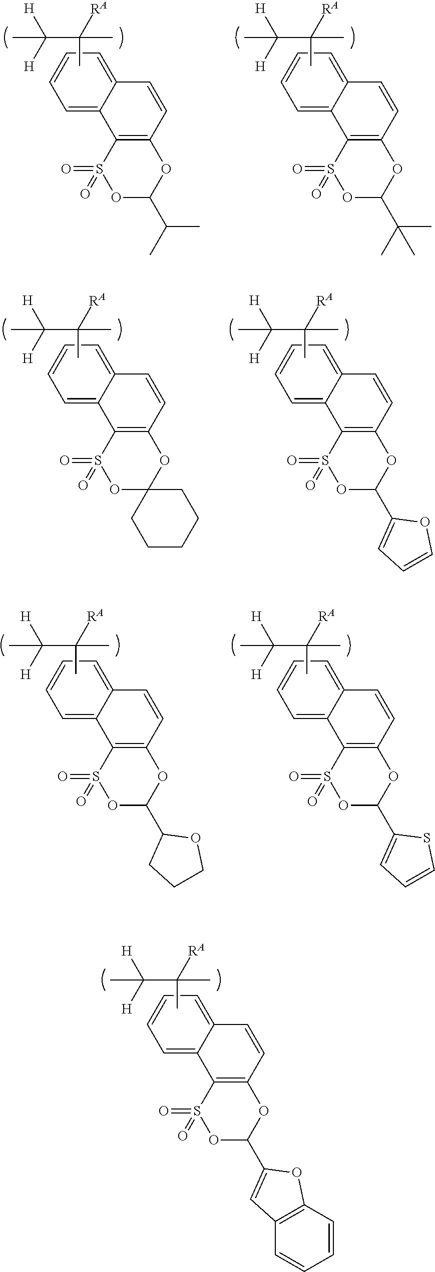

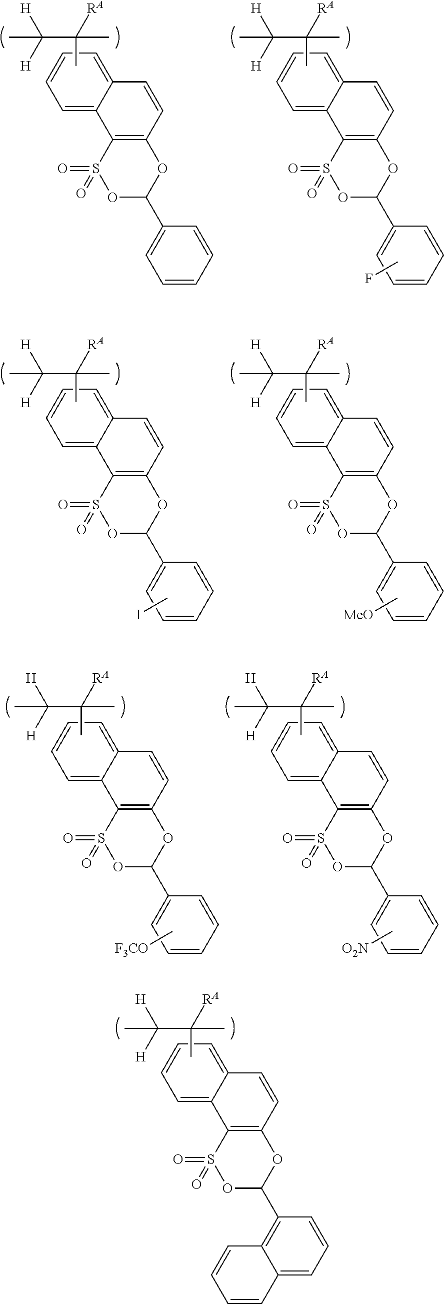

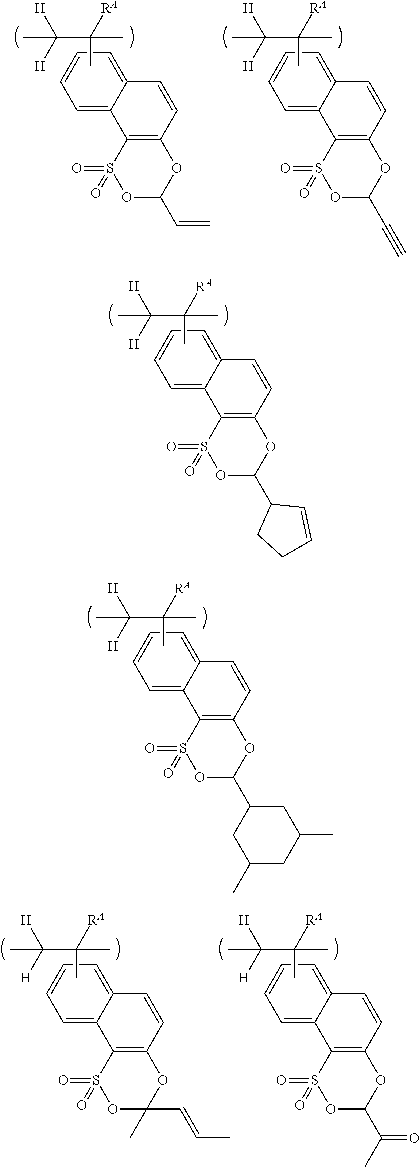

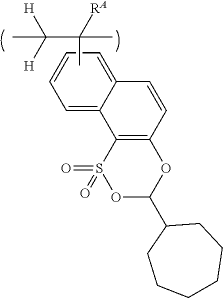

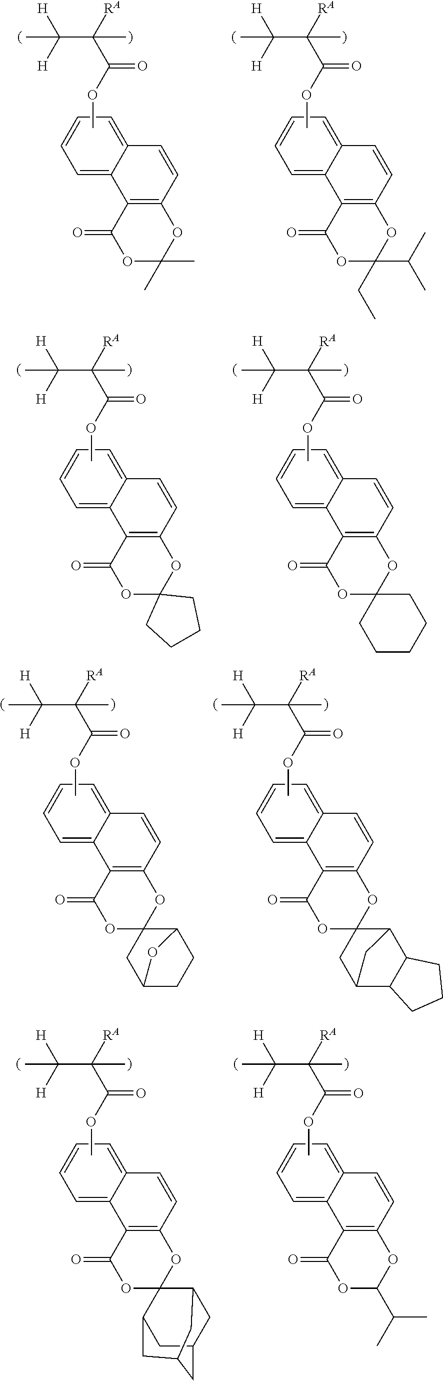

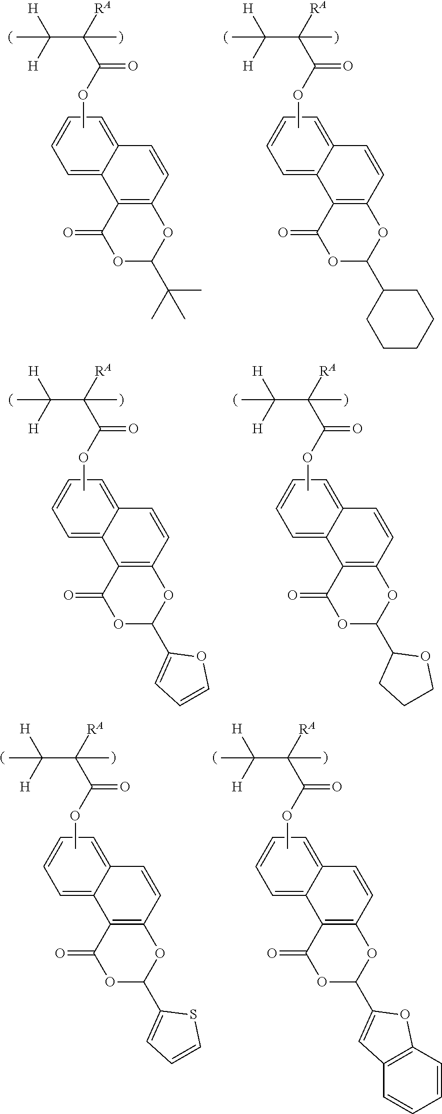

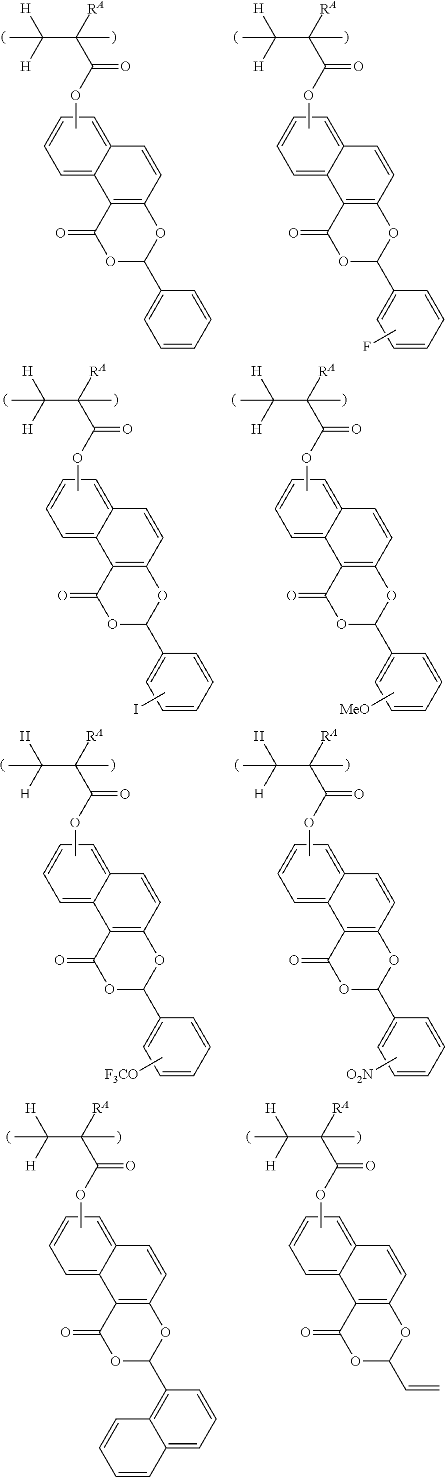

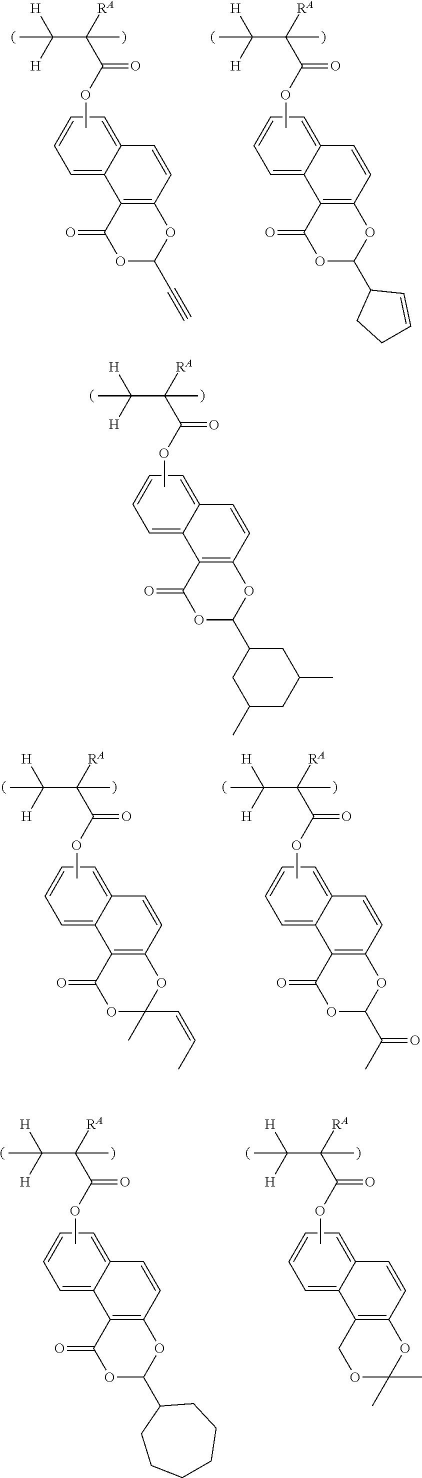

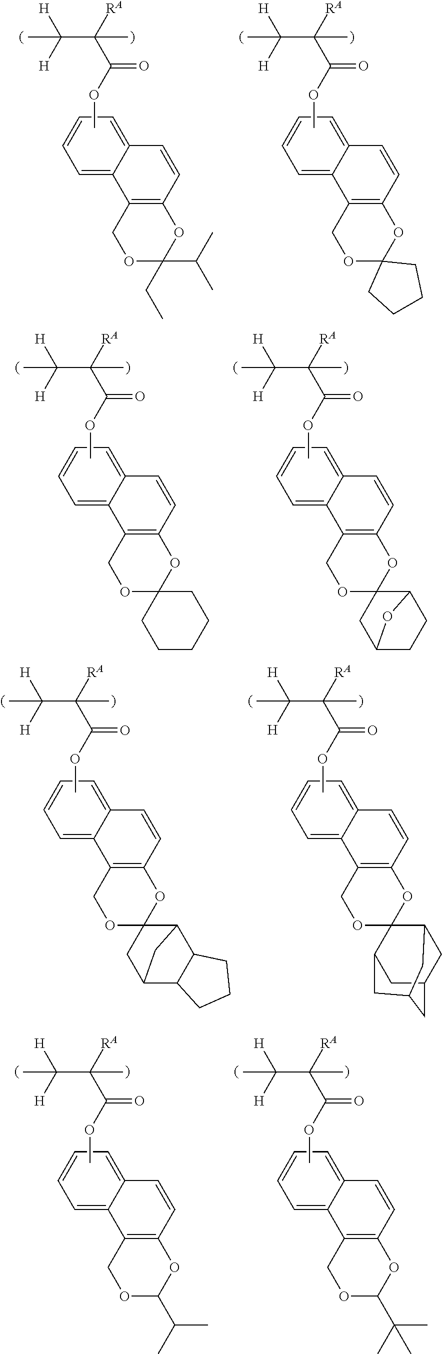

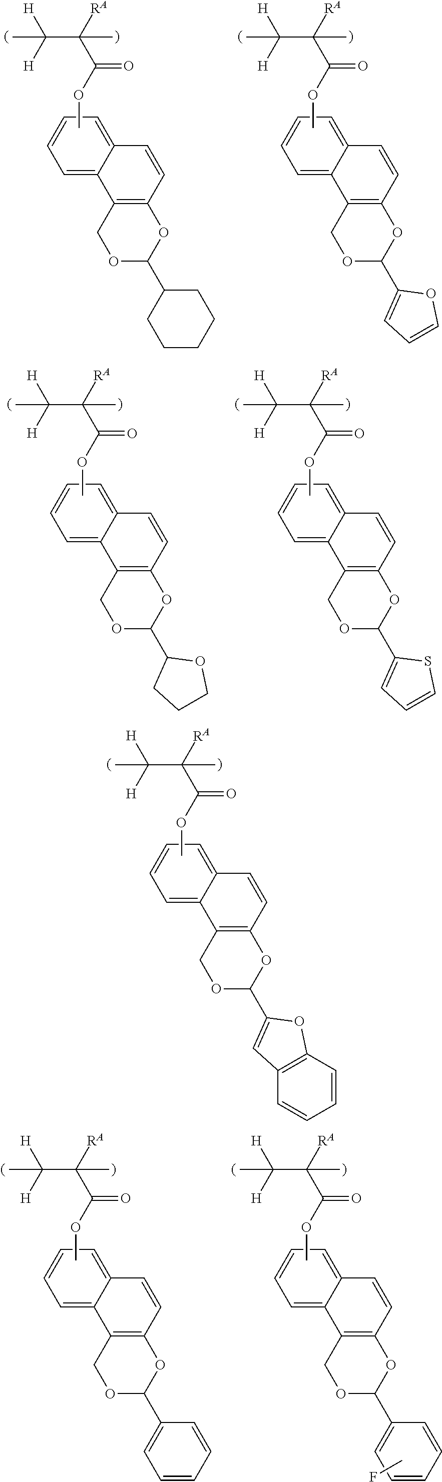

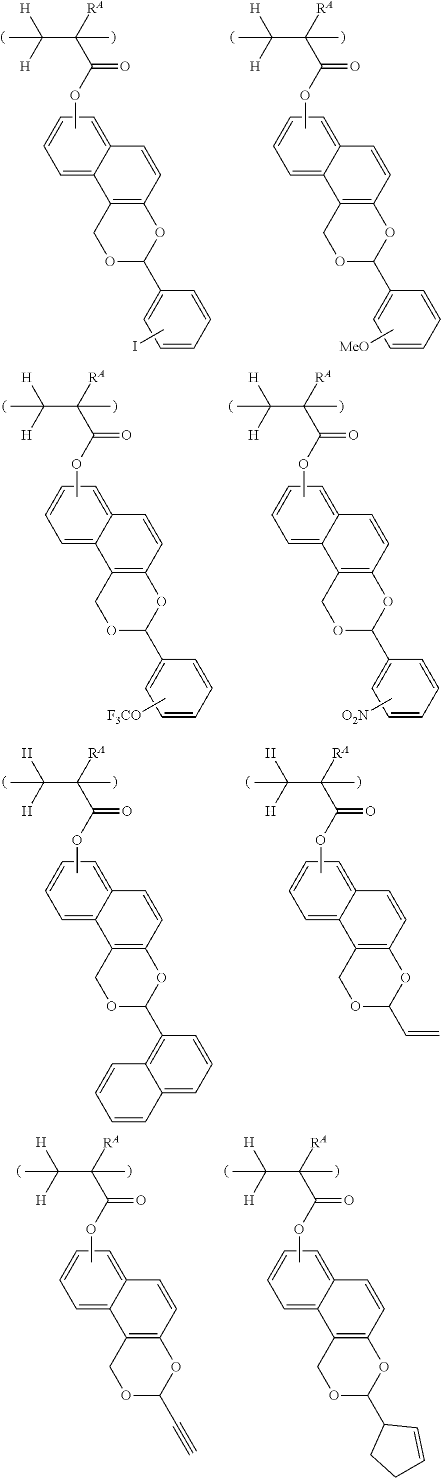

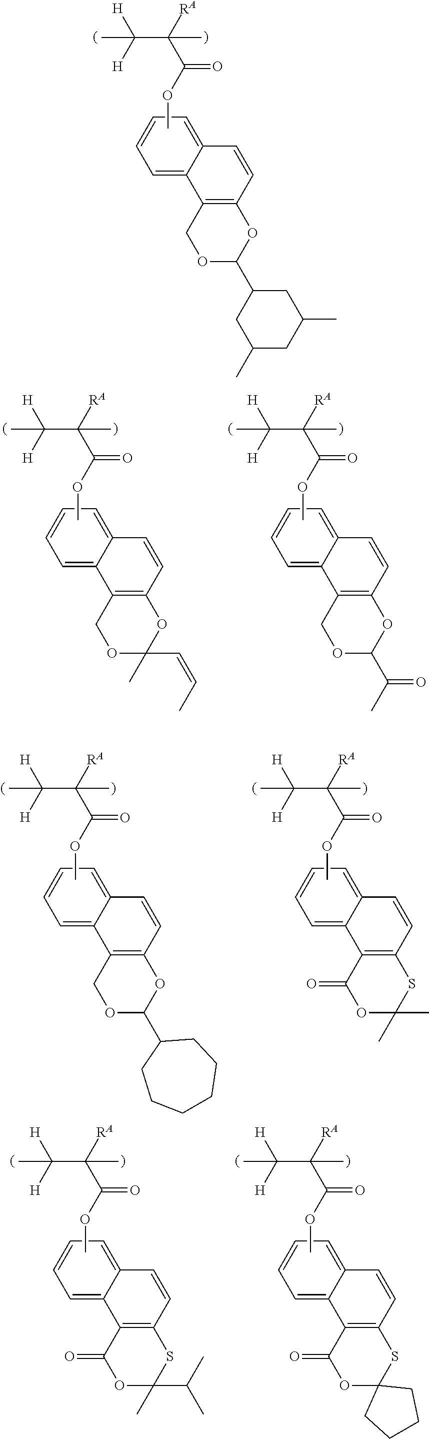

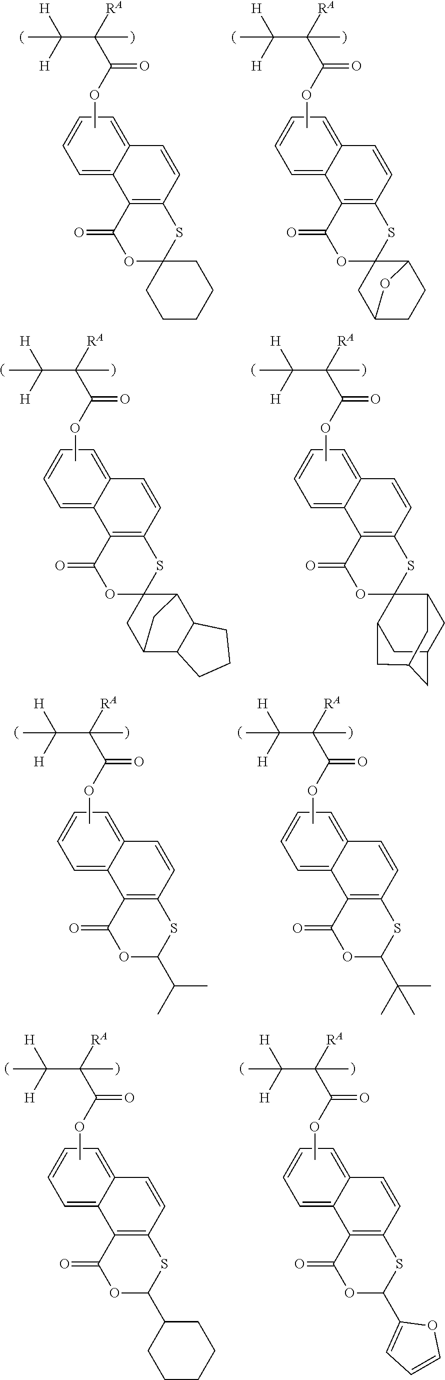

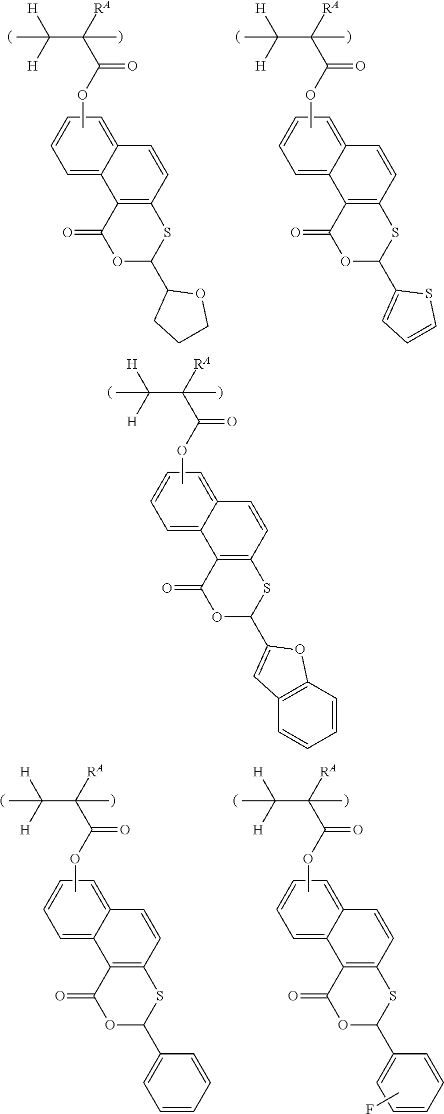

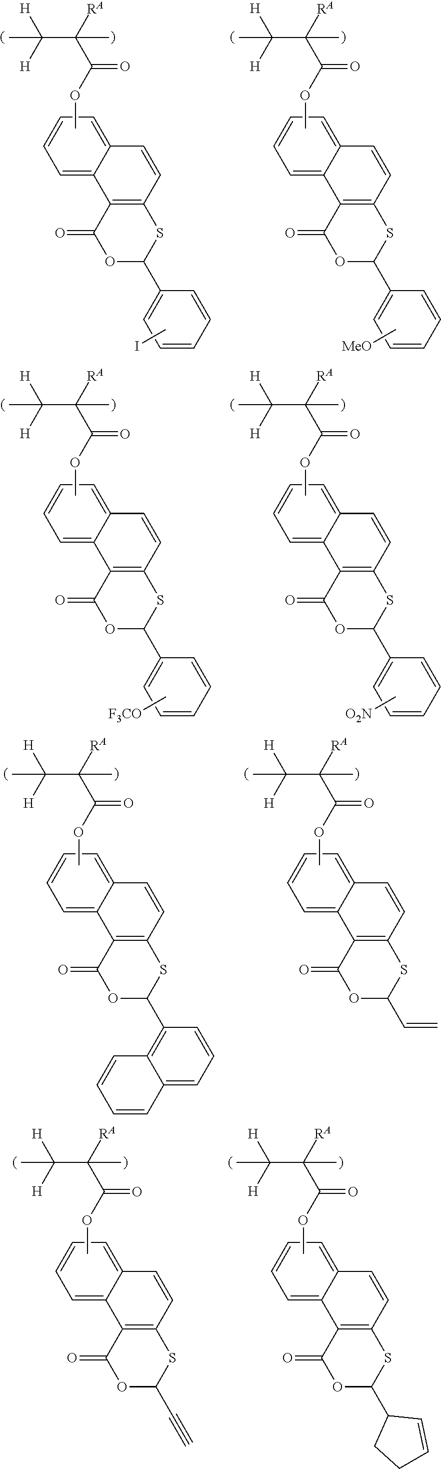

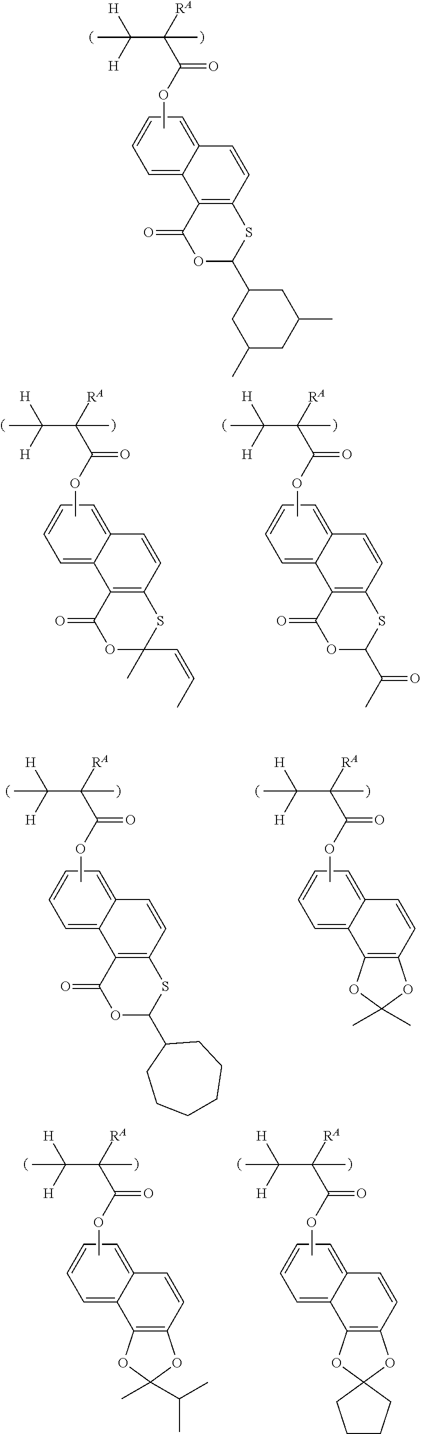

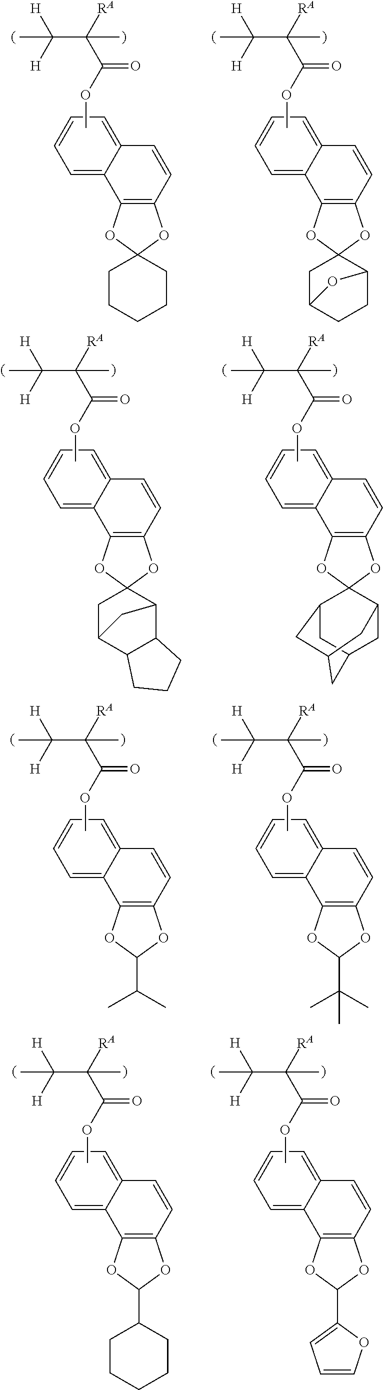

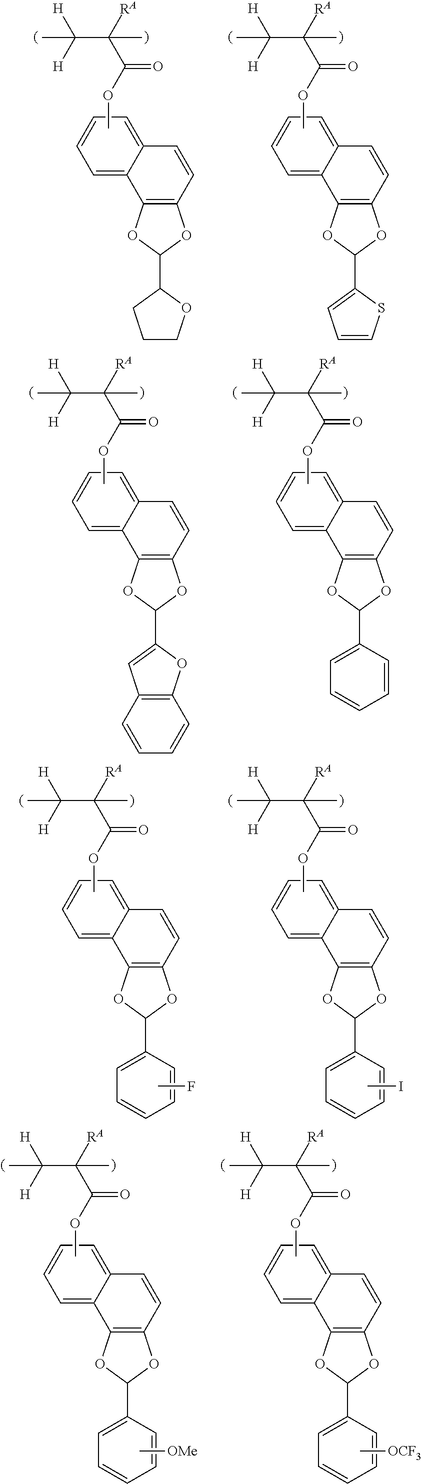

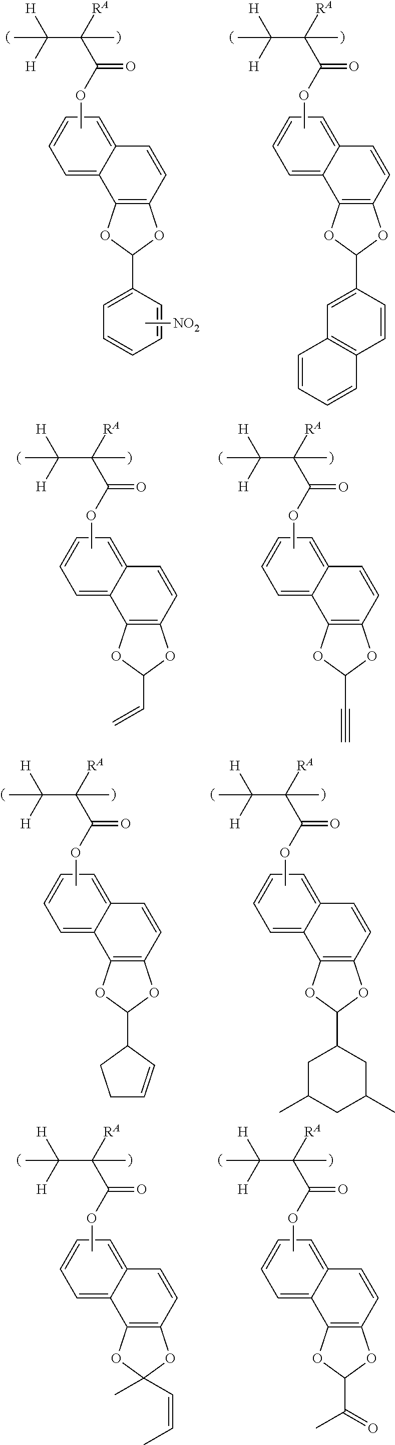

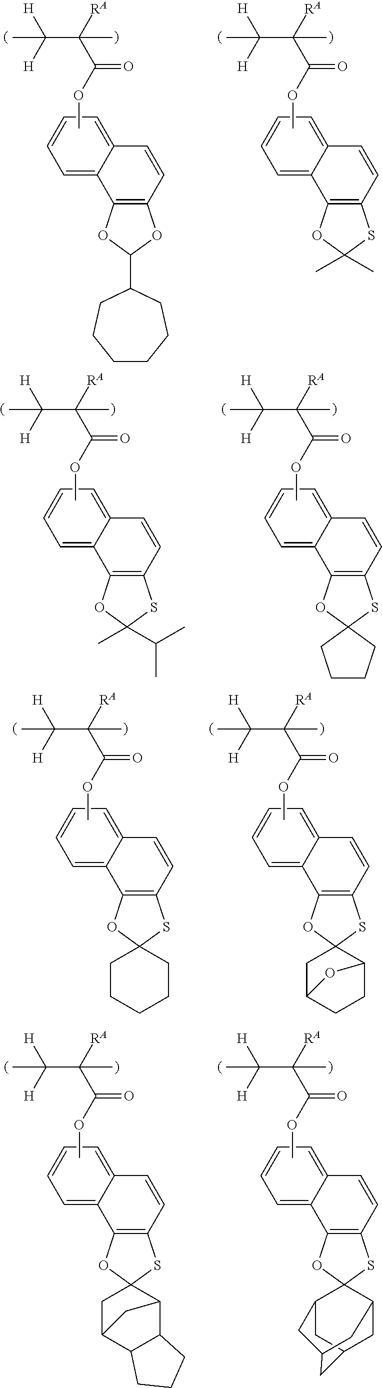

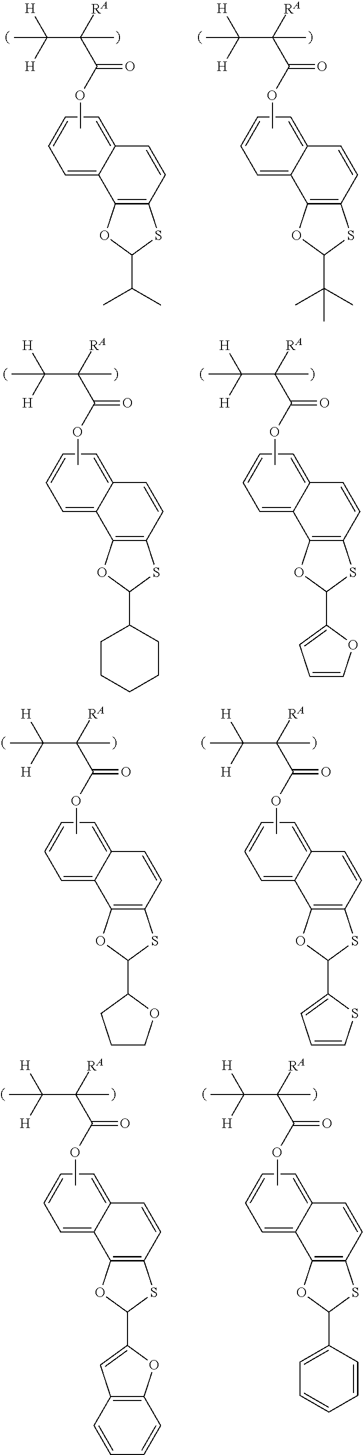

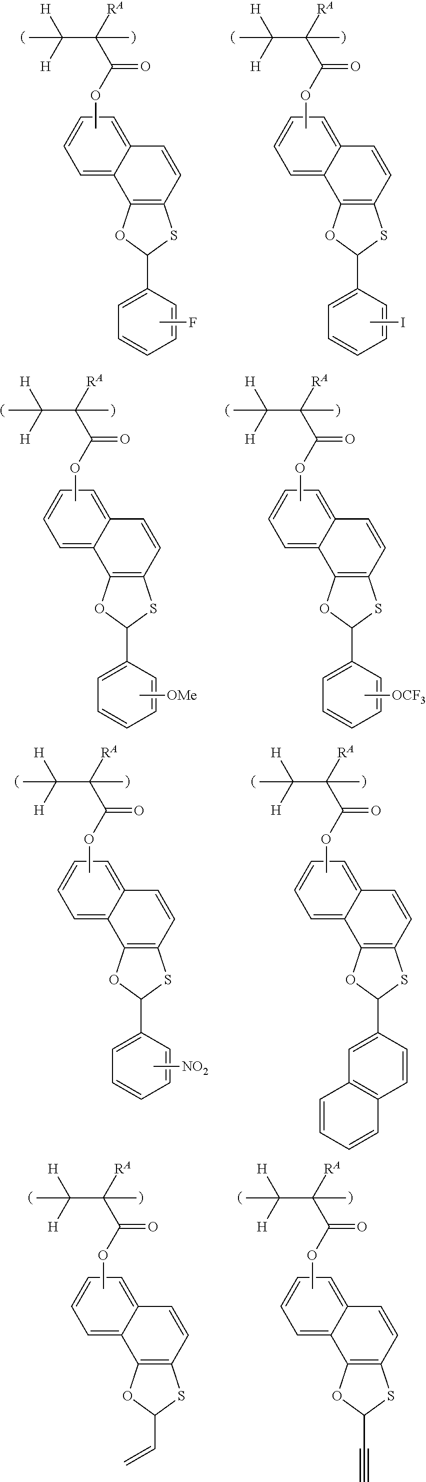

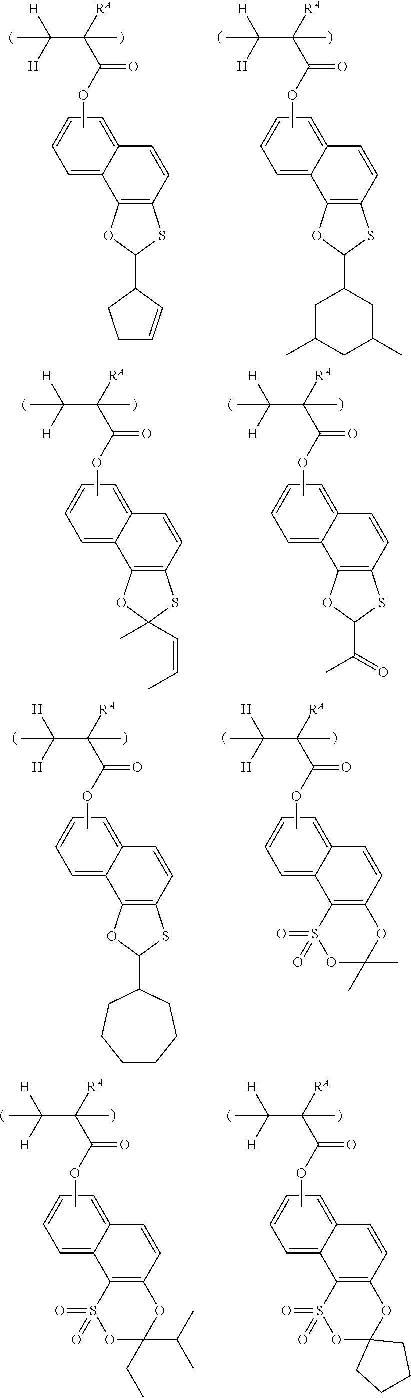

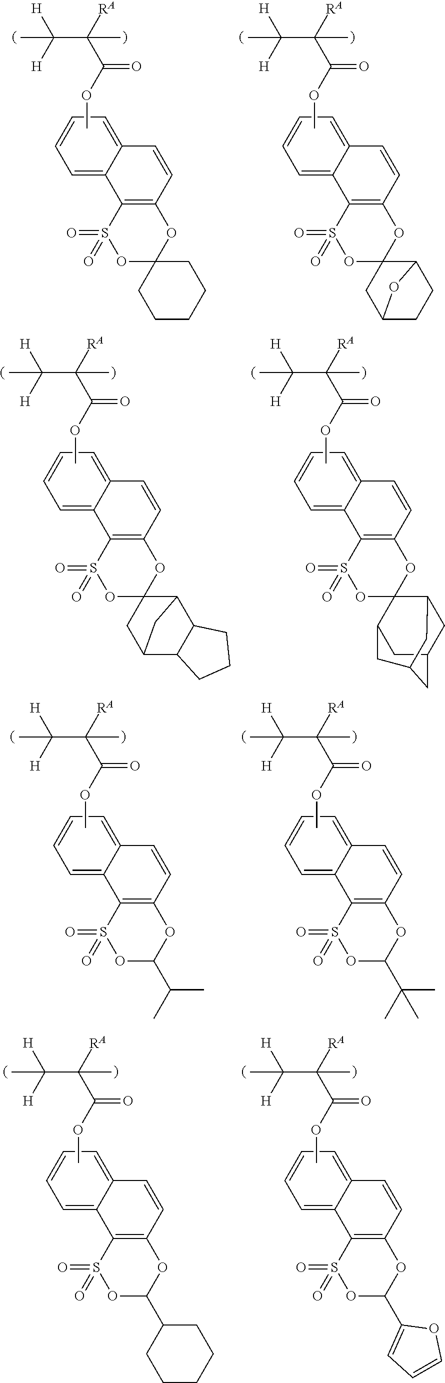

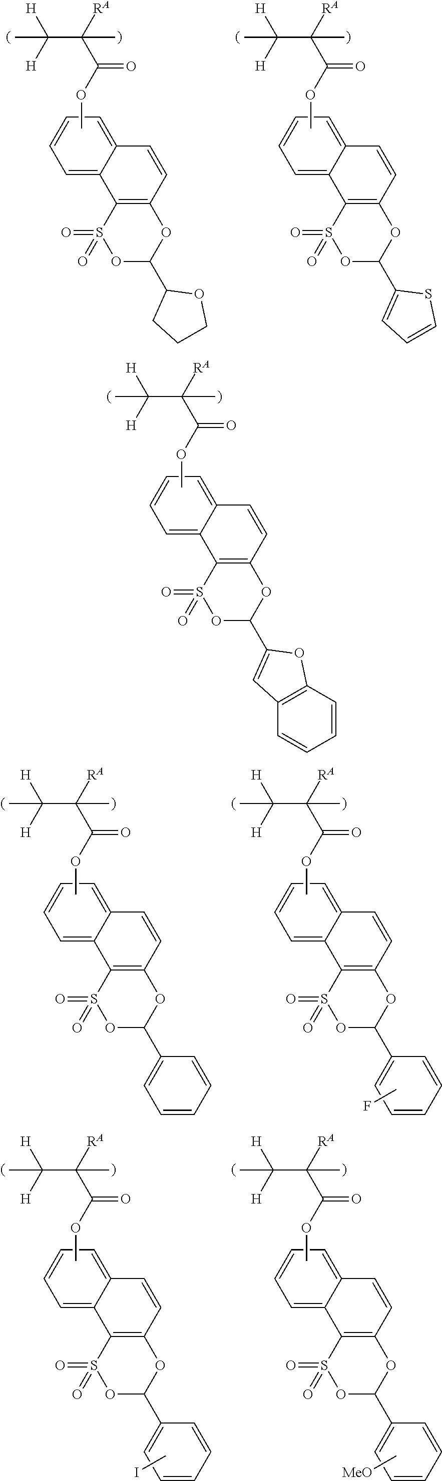

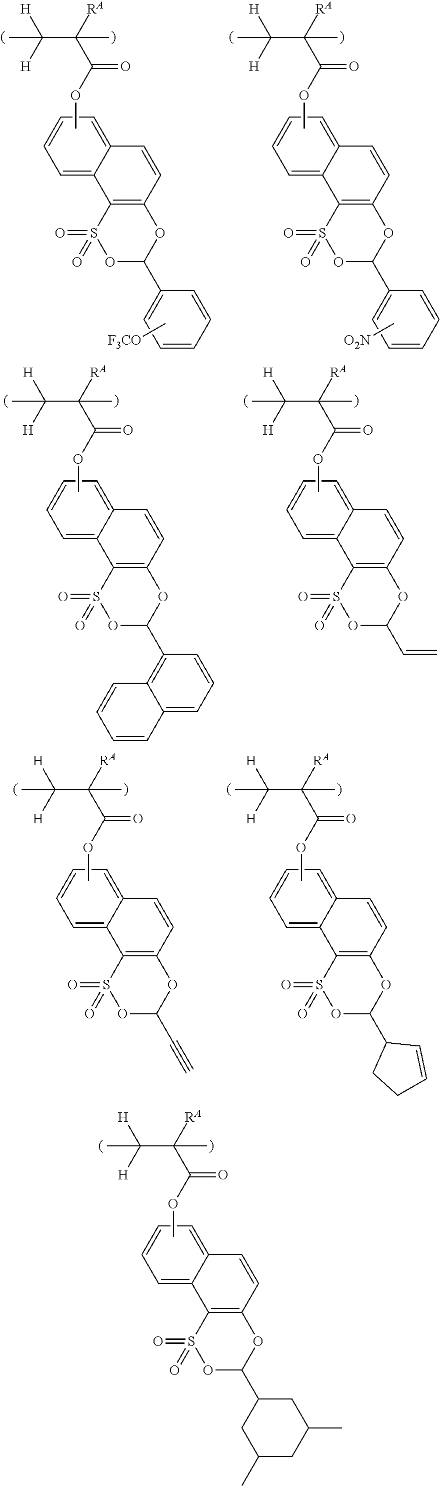

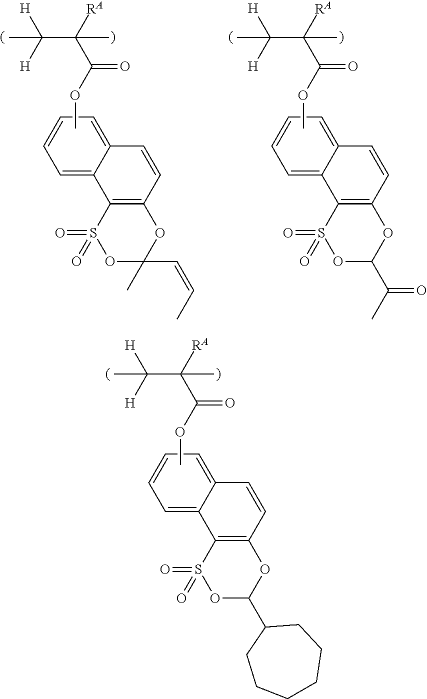

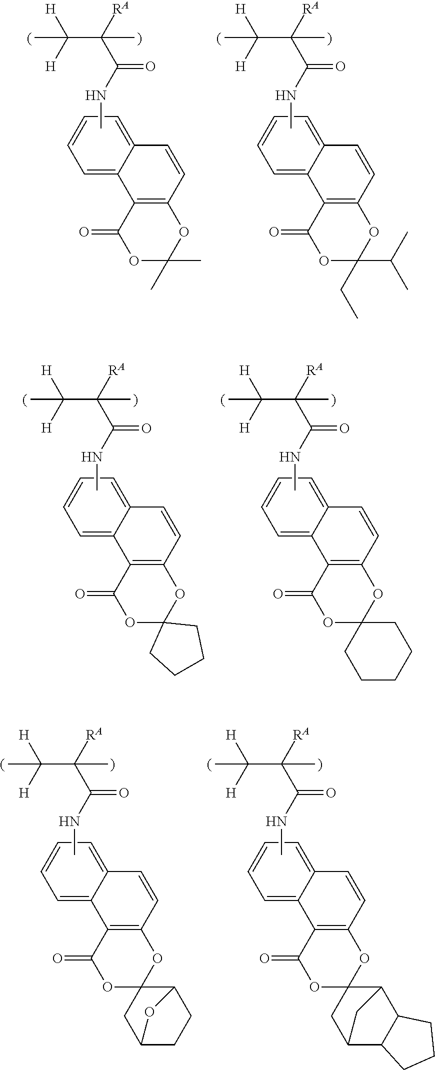

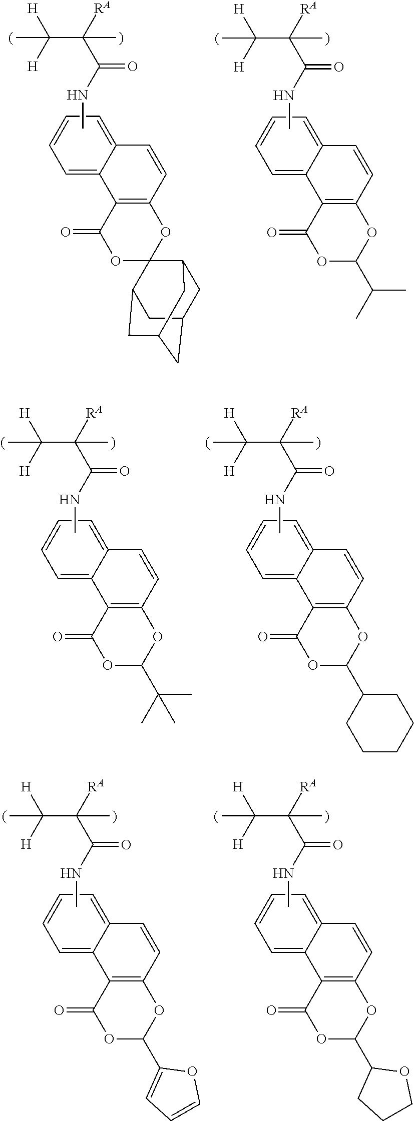

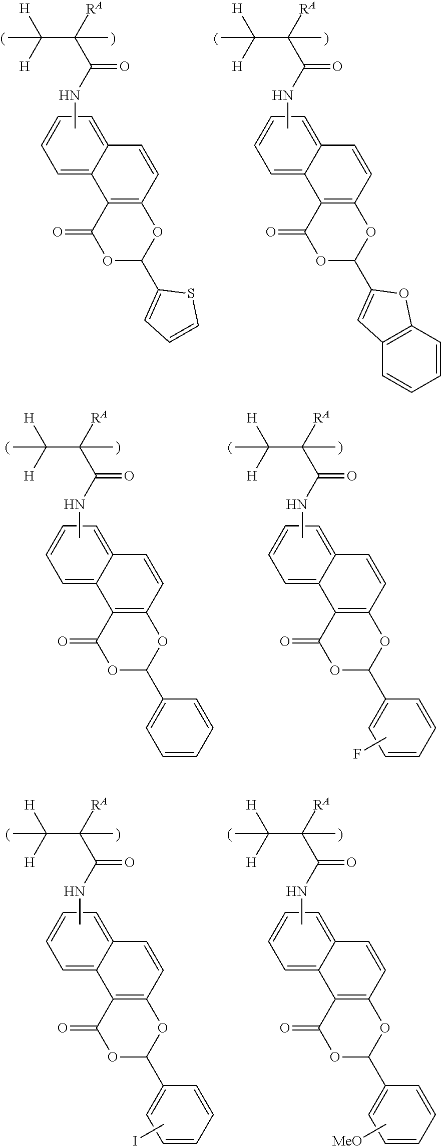

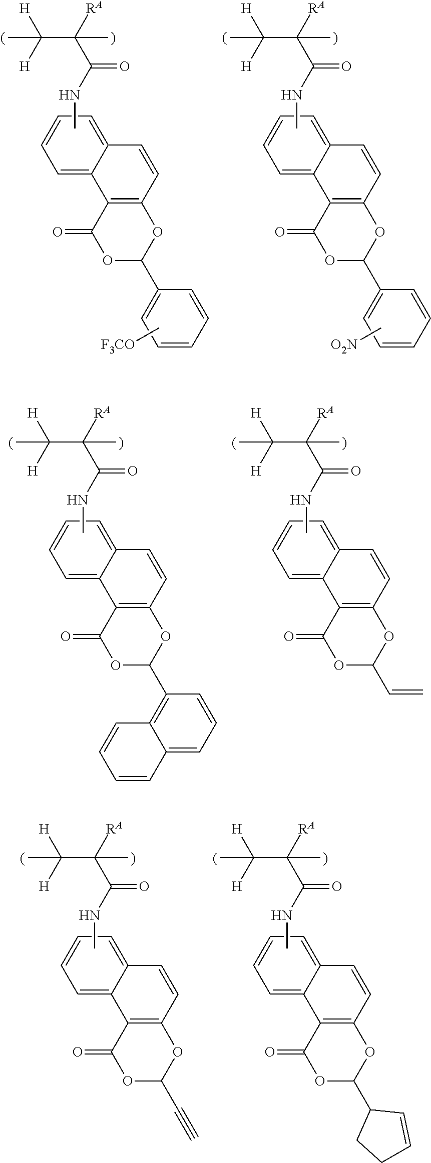

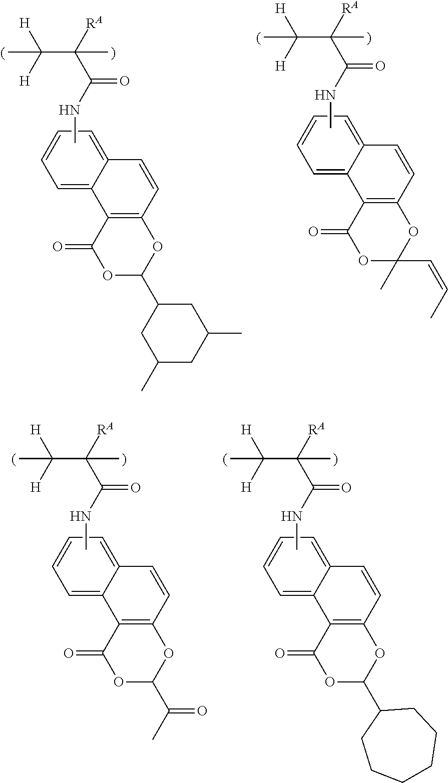

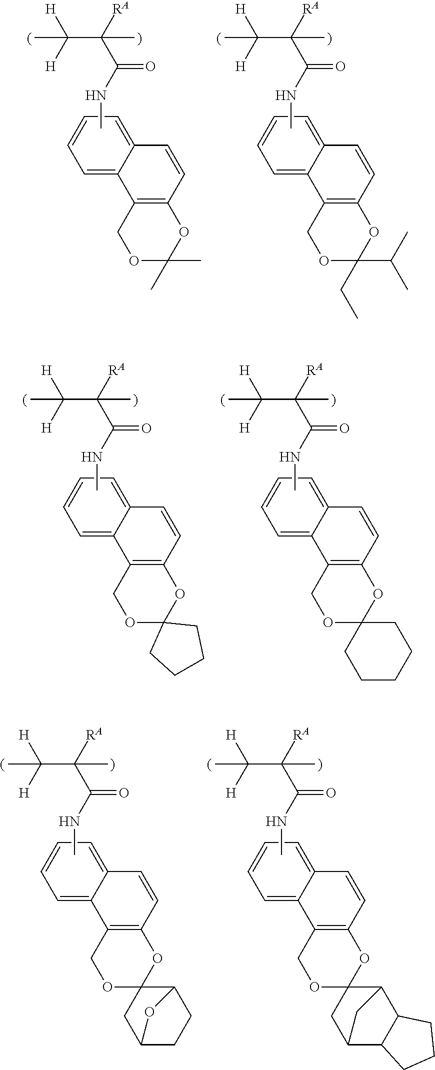

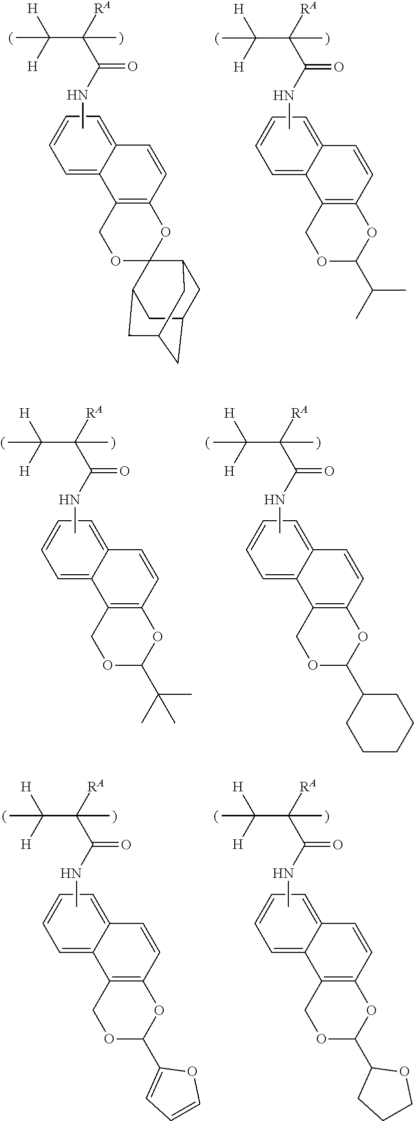

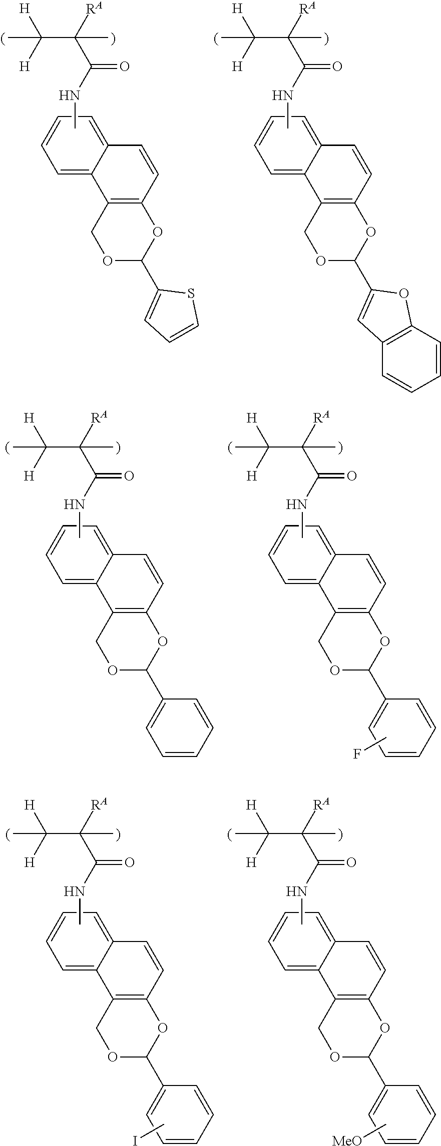

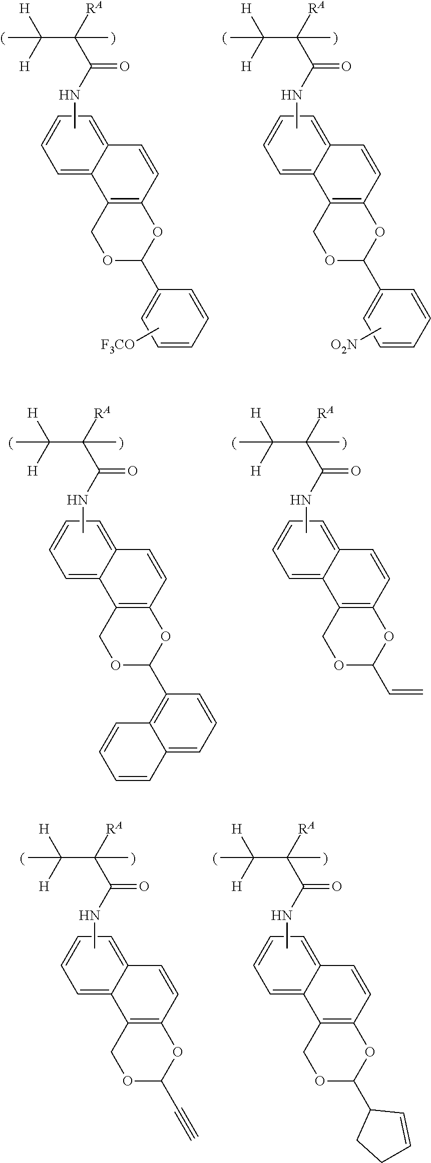

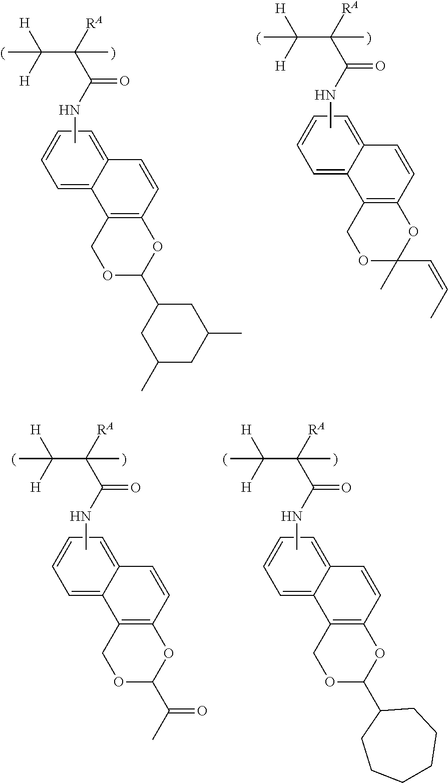

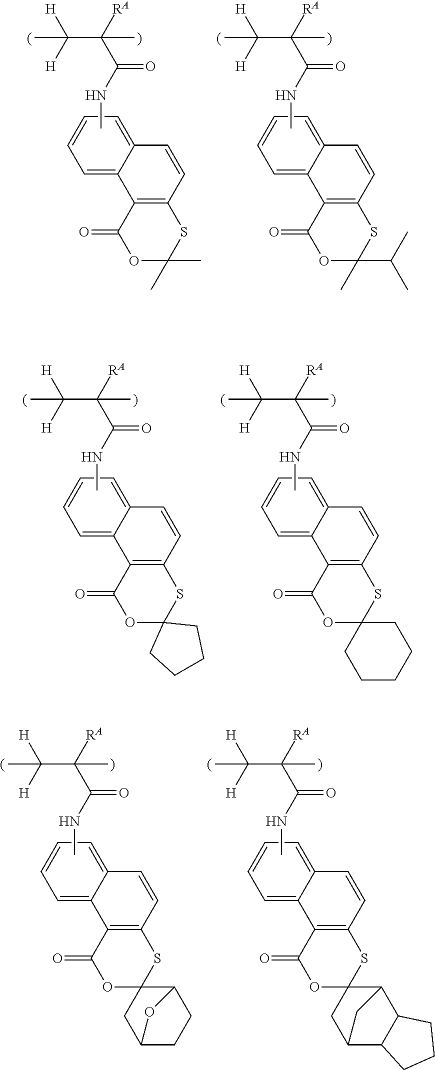

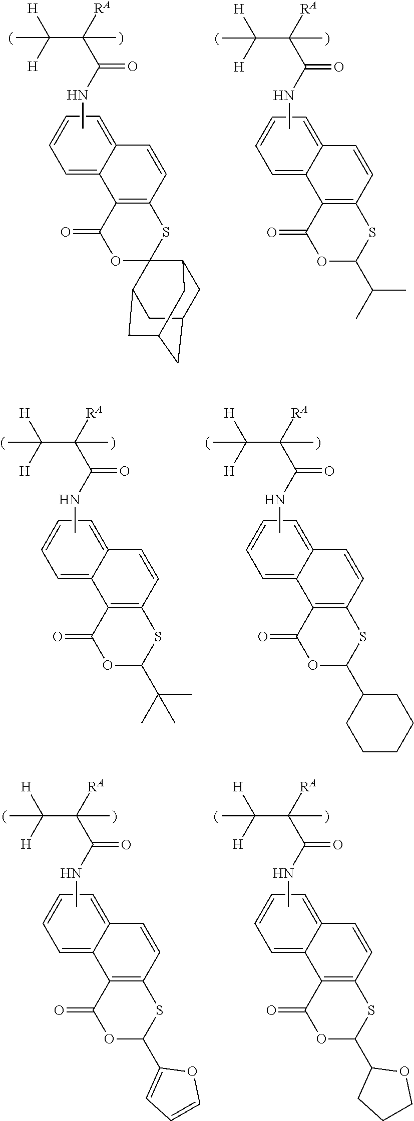

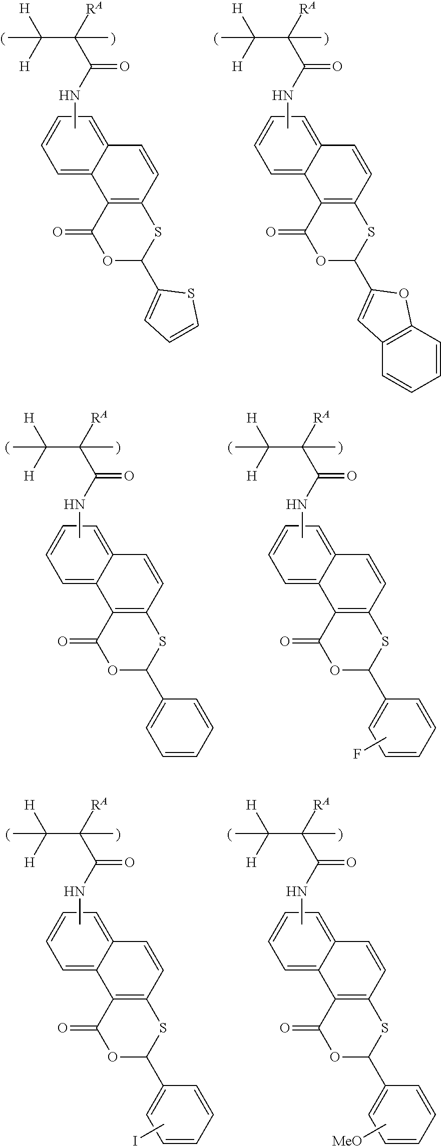

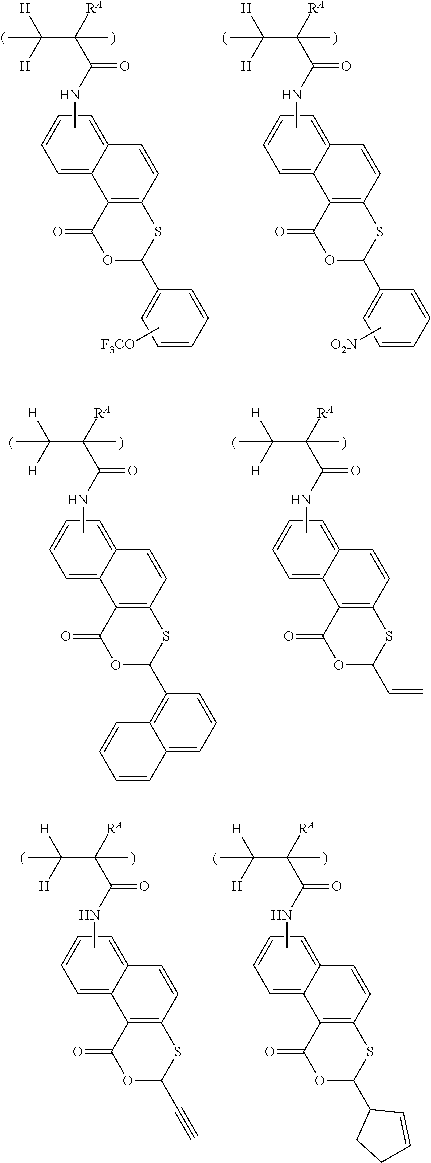

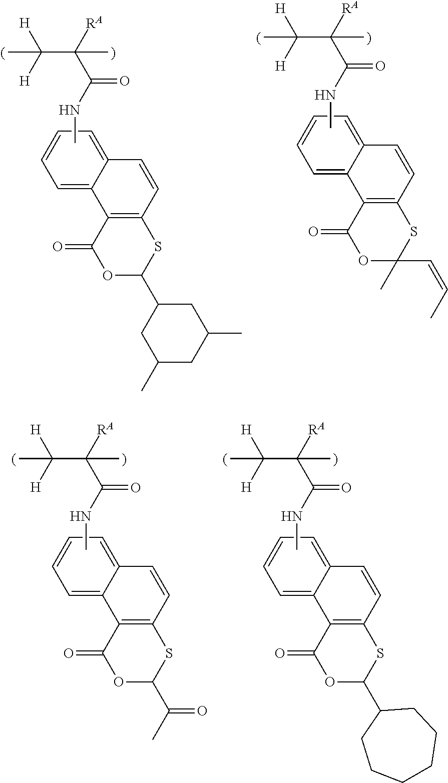

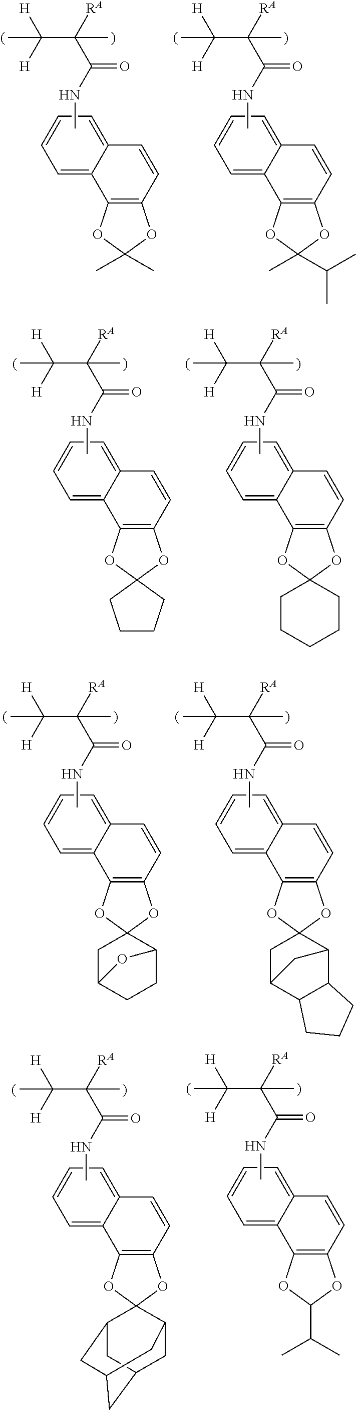

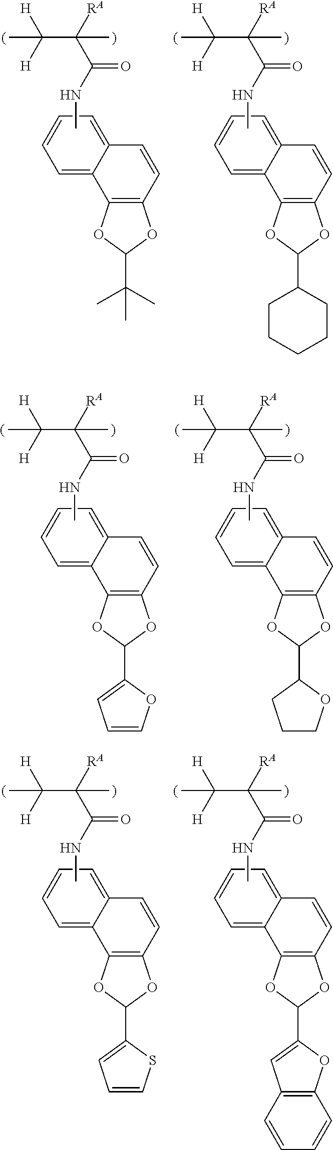

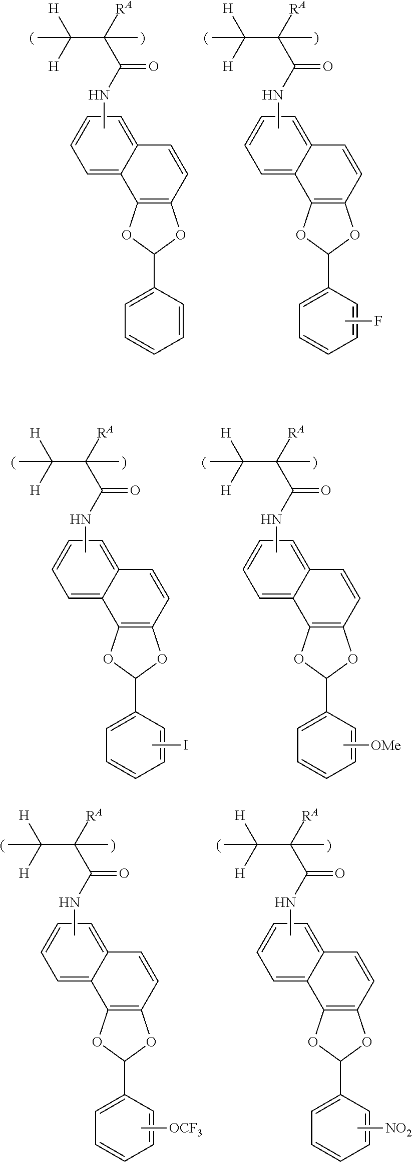

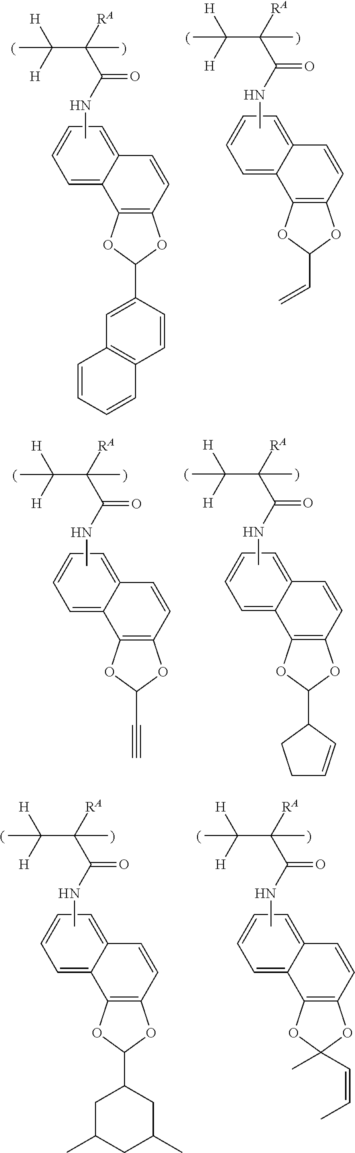

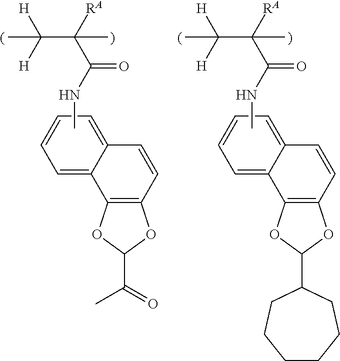

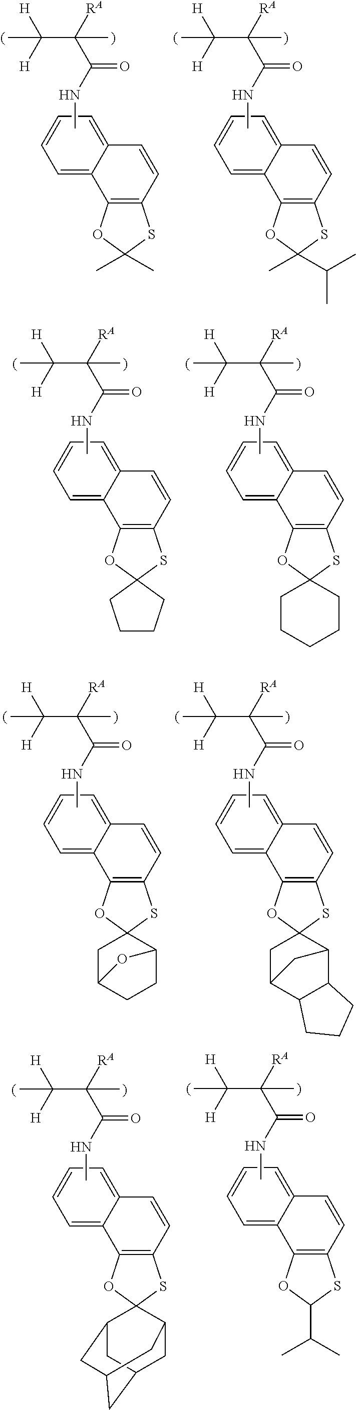

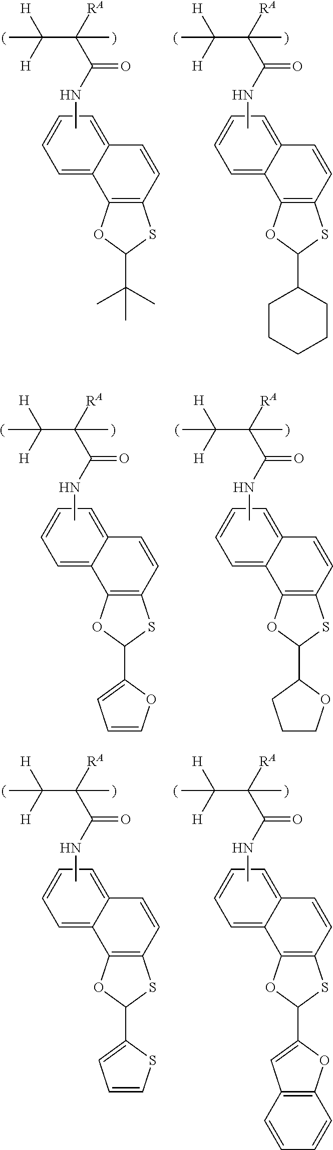

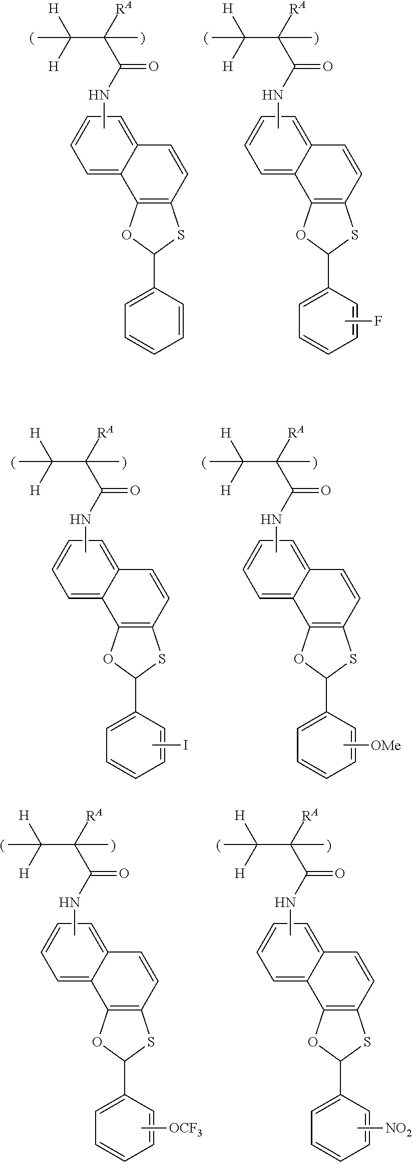

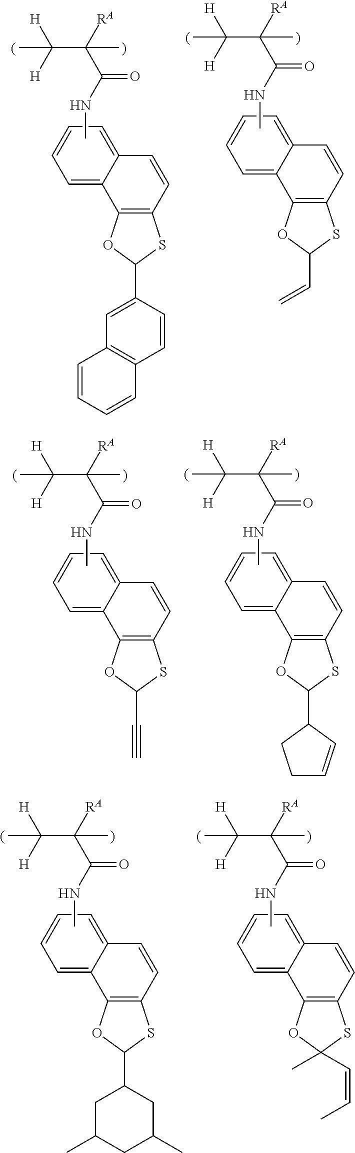

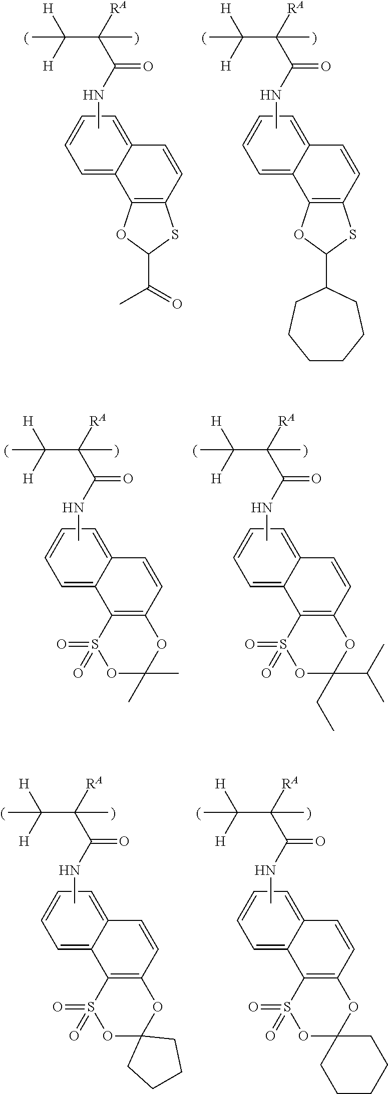

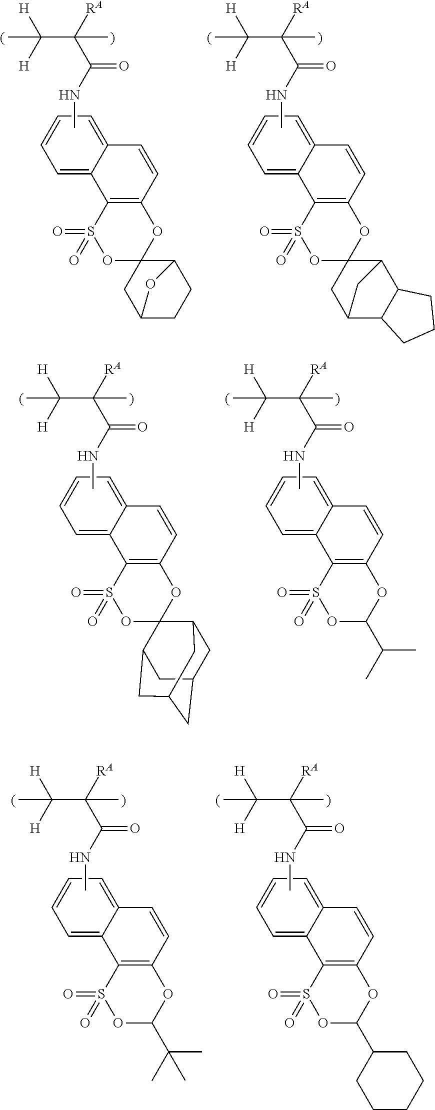

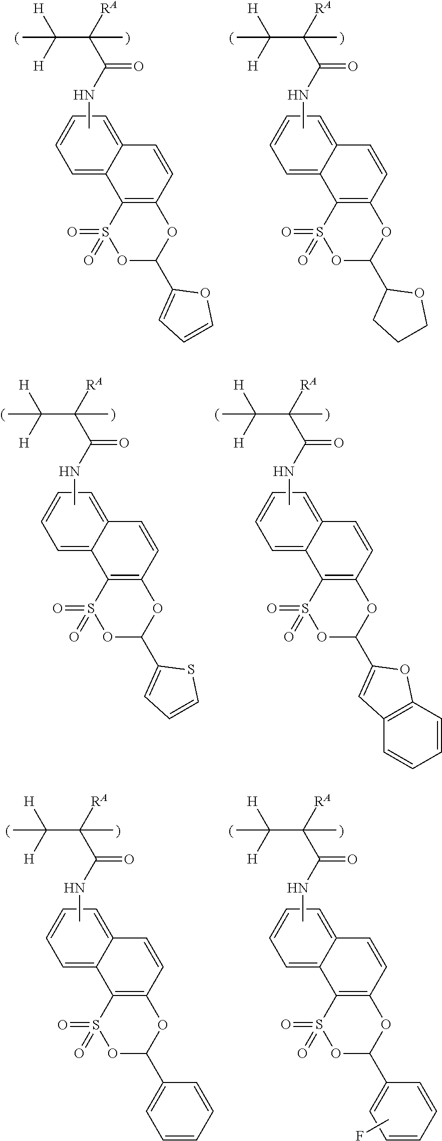

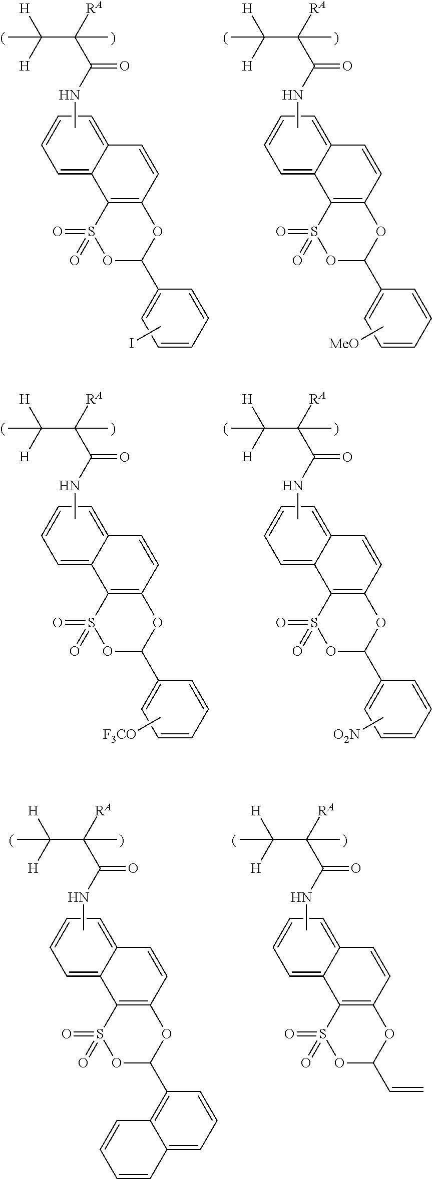

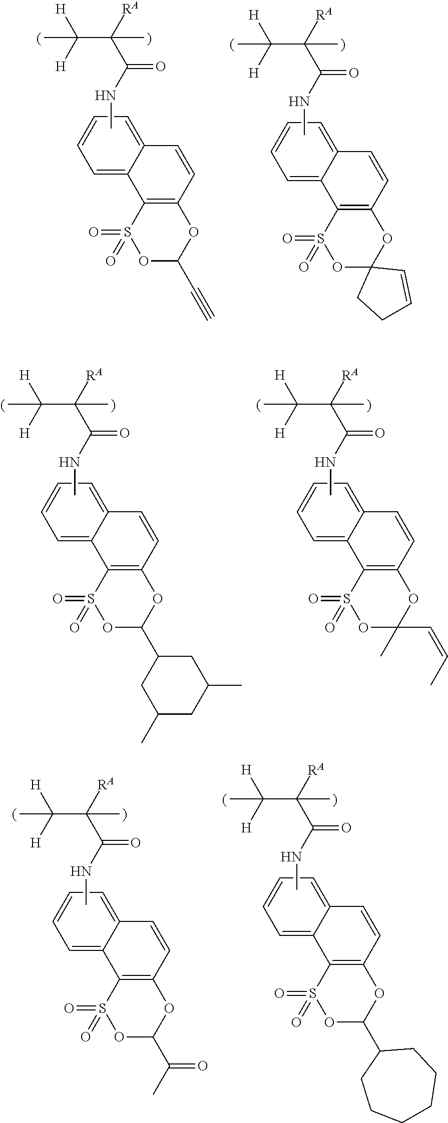

In a preferred embodiment, the base polymer further comprises repeat units having the formula (b1) or repeat units having the formula (b2), which are simply referred to as repeat units (b1) or (b2).

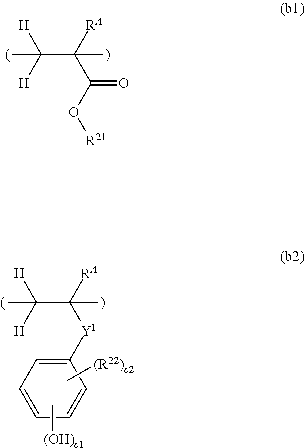

In formulae (b1) and (b2), R^{A} is each independently hydrogen, fluorine, methyl or trifluoromethyl. Y¹ is a single bond or *—C(═O)—O—. The asterisk (*) designates a point of attachment to the carbon atom in the backbone. R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—). R²² is halogen, hydroxy, carboxy, nitro, cyano, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom or C₂-C₂₀ hydrocarbyloxycarbonyl group which may contain a heteroatom. A plurality of R²² may be identical or different when c2 is 2, 3 or 4. The subscript c1 is 1, 2, 3 or 4. The subscript c2 is 0, 1, 2, 3 or 4. It is noted that c1+c2 is from 1 to 5.

Examples of repeat unit (b1) are shown below, but not limited thereto. Herein R^{A} is as defined above.

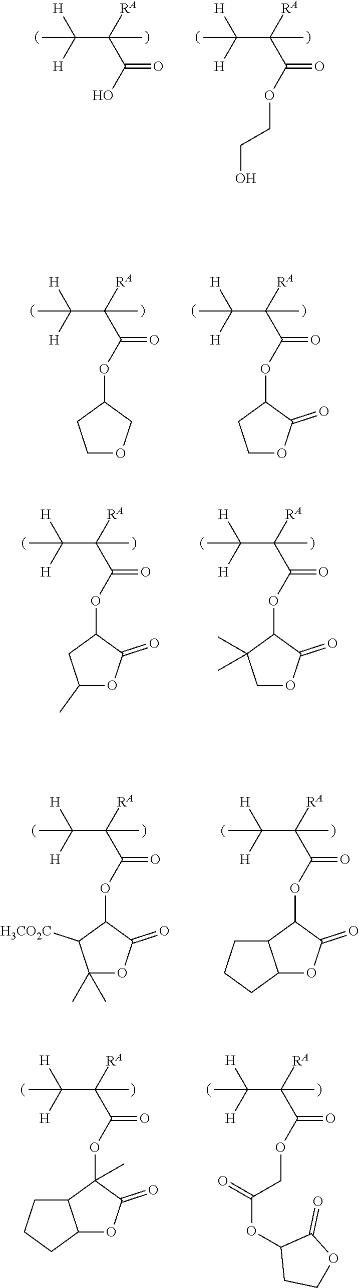

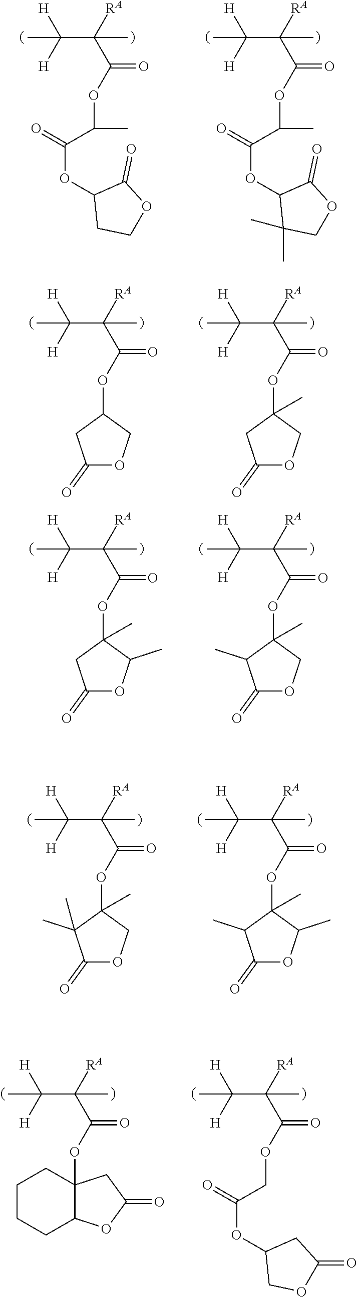

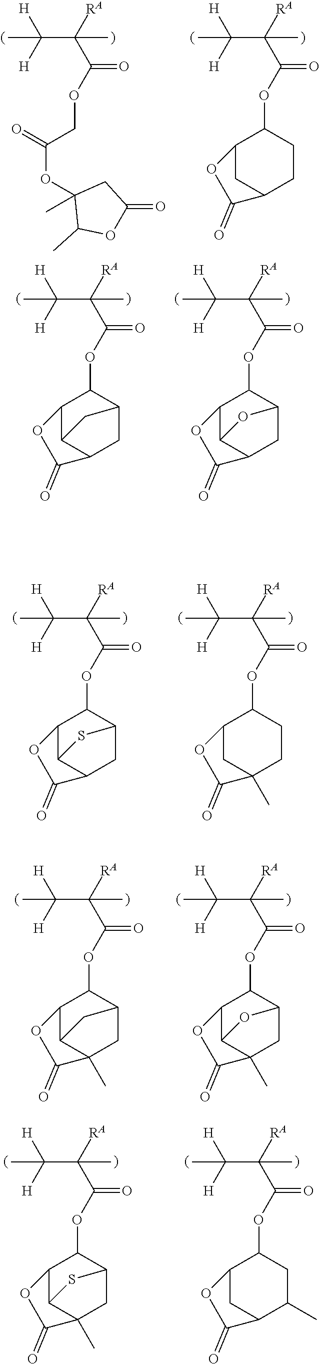

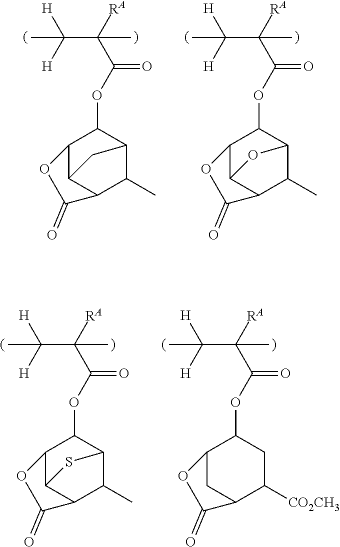

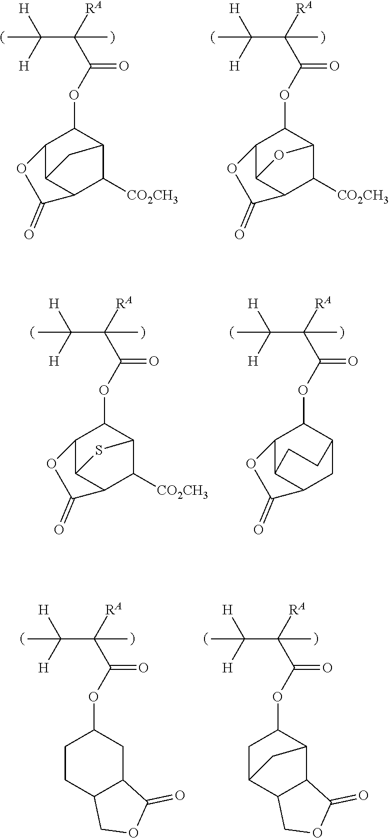

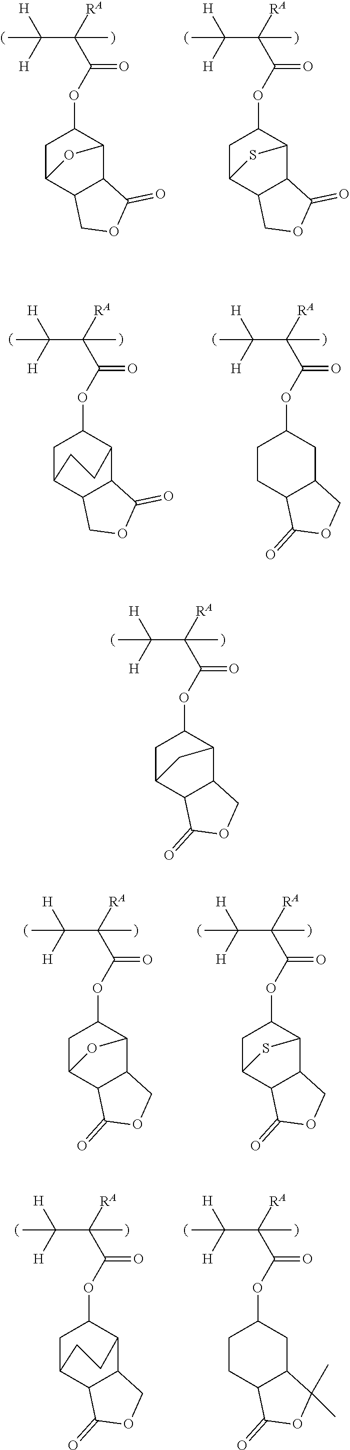

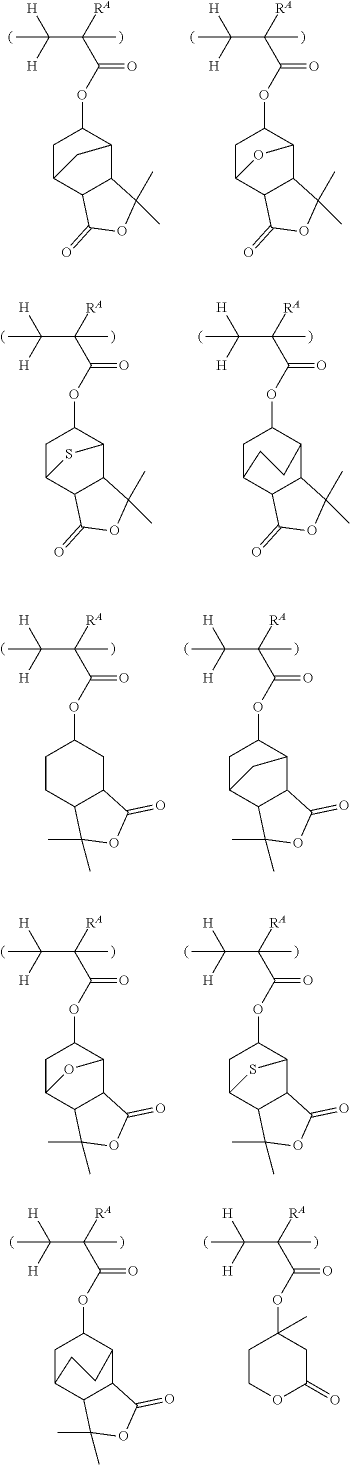

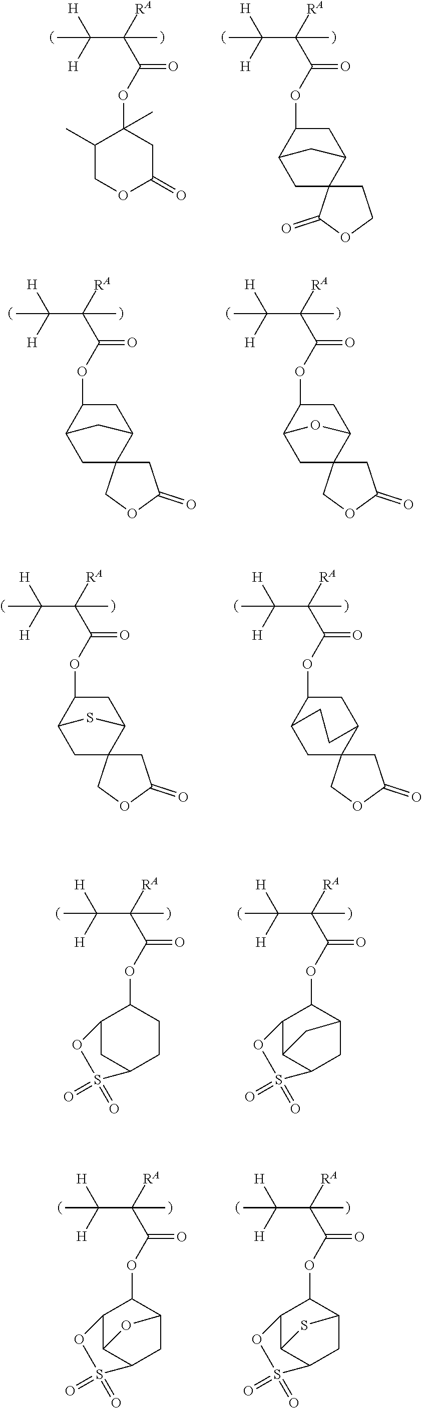

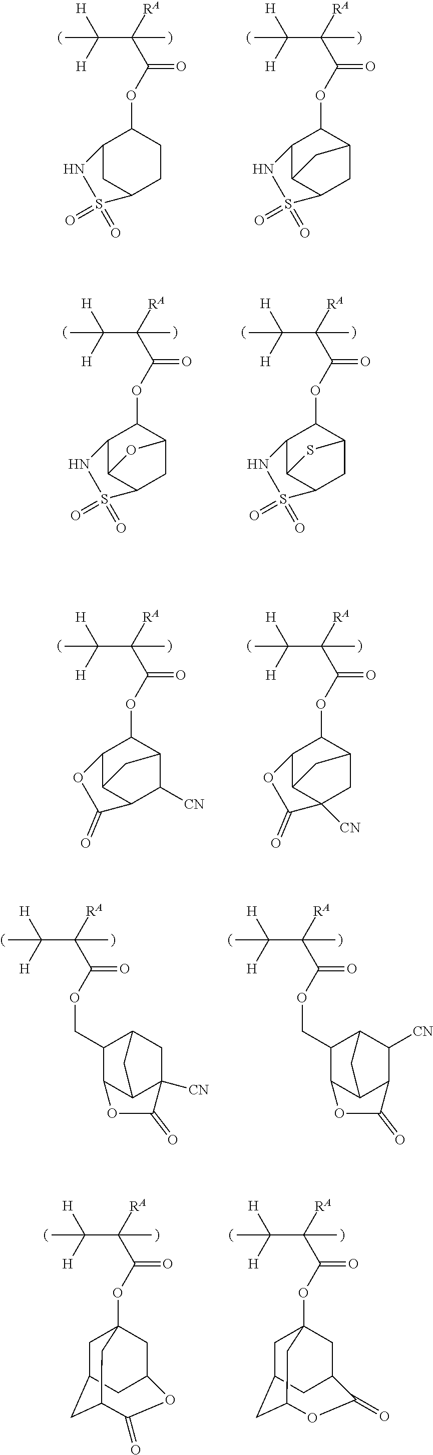

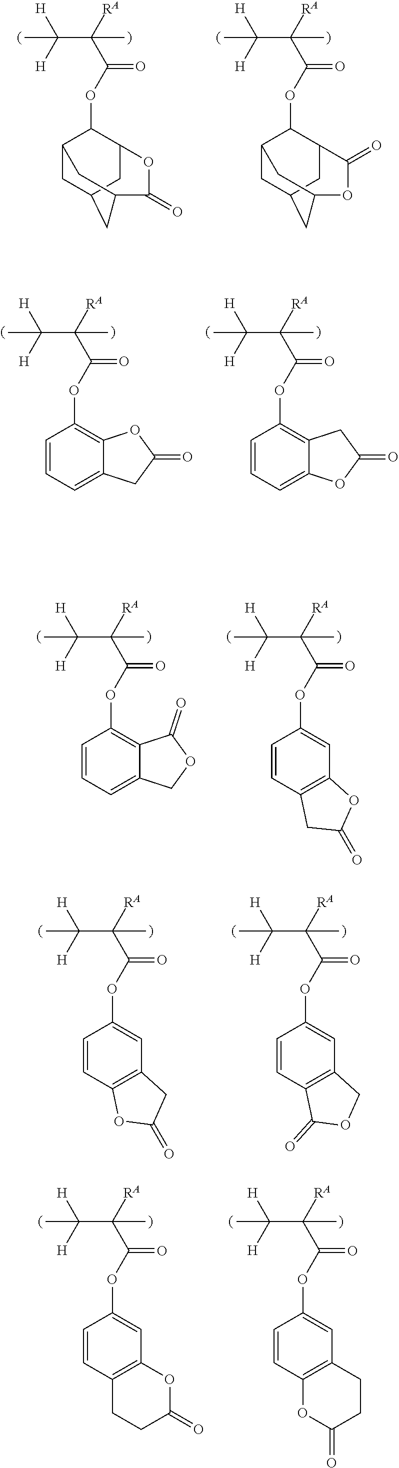

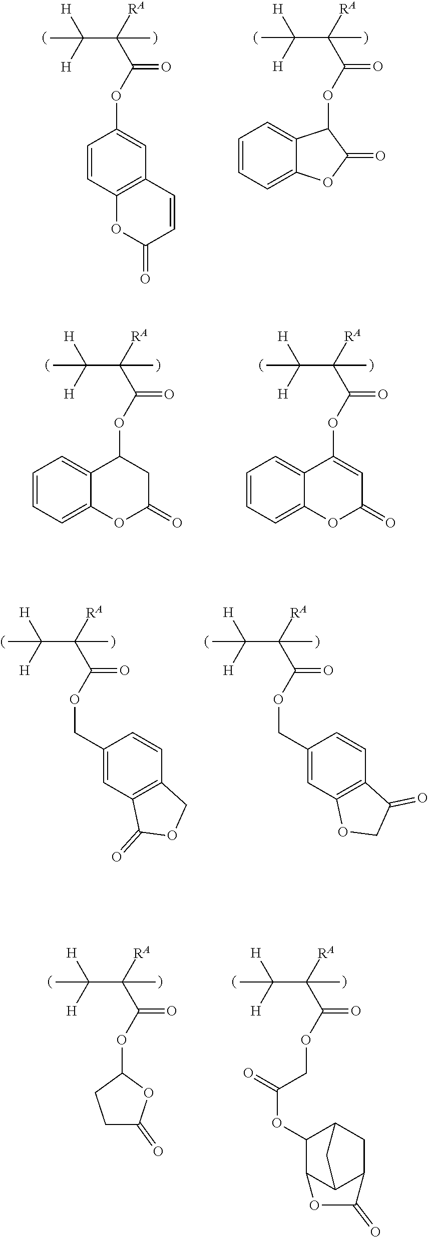

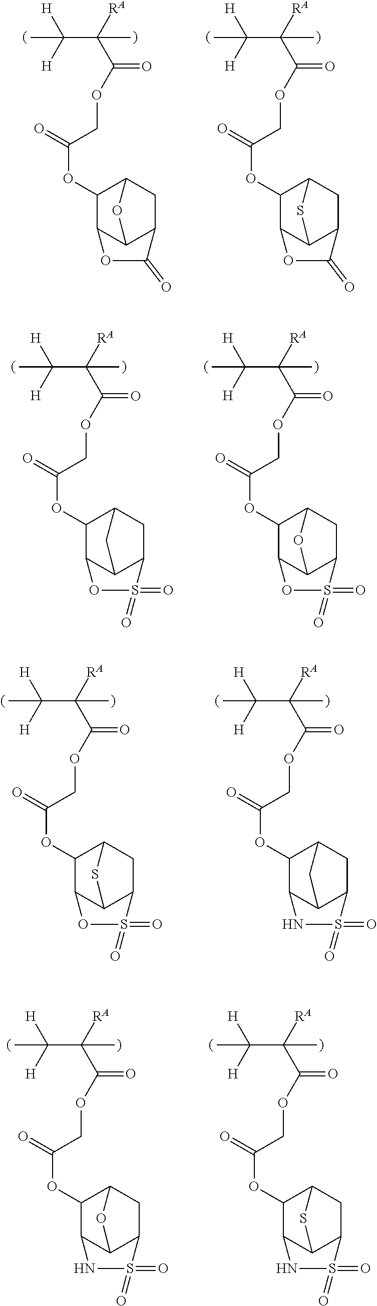

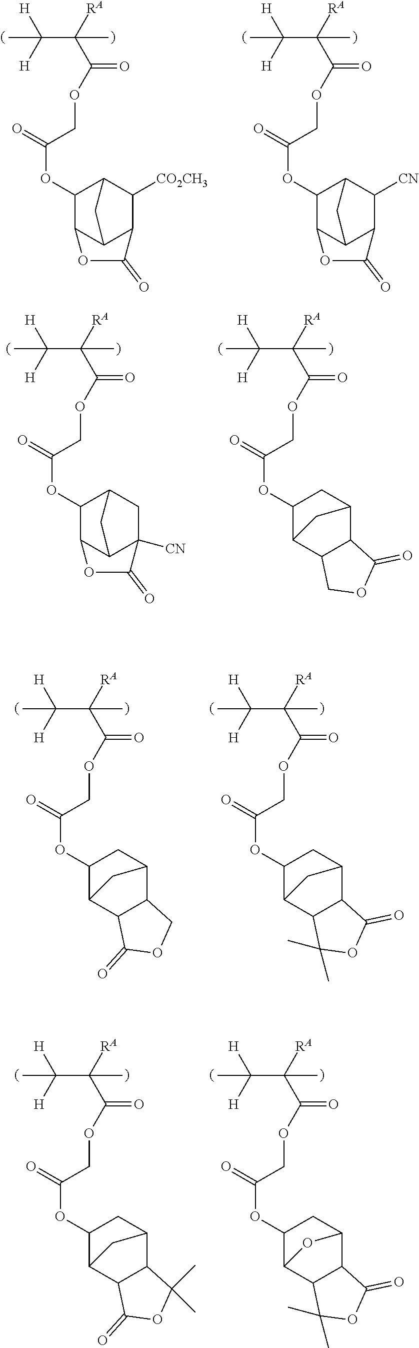

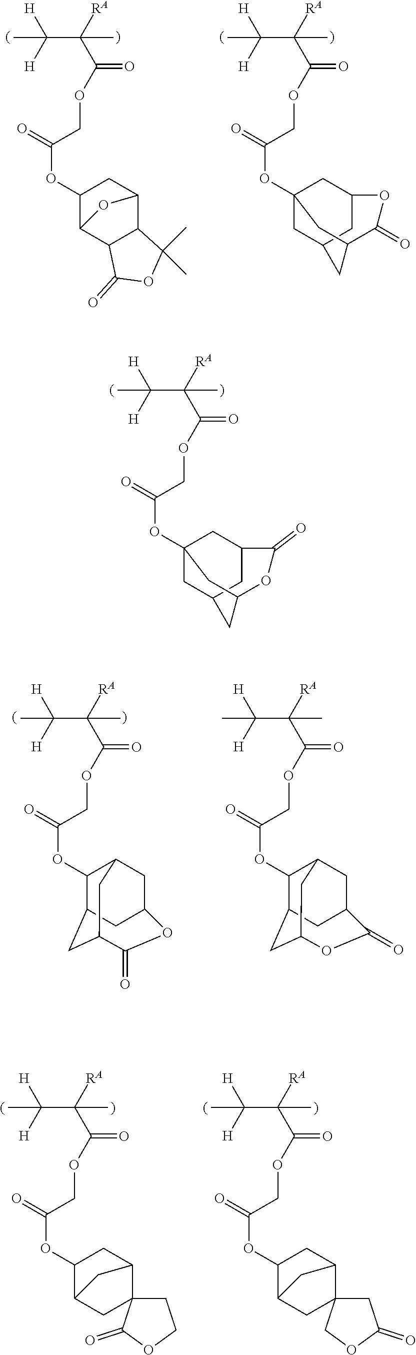

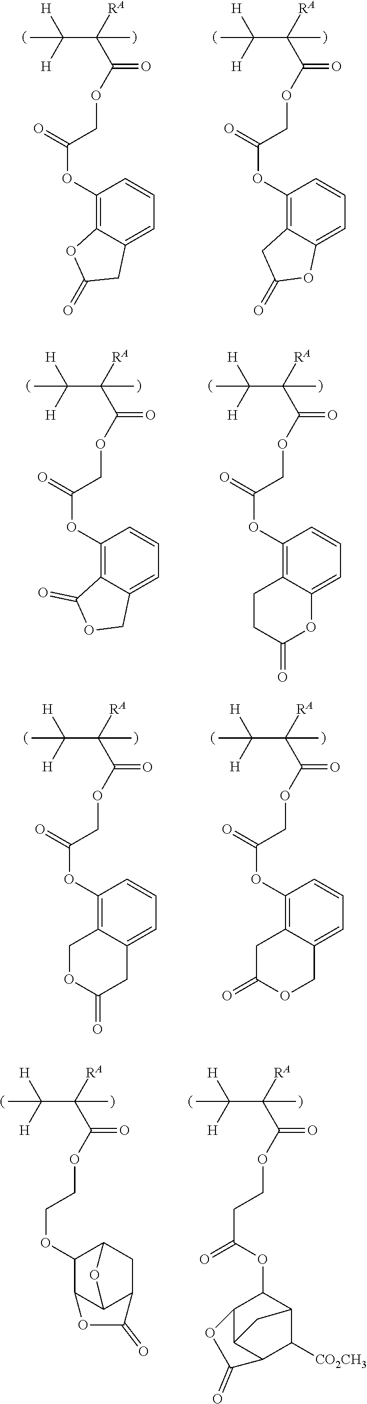

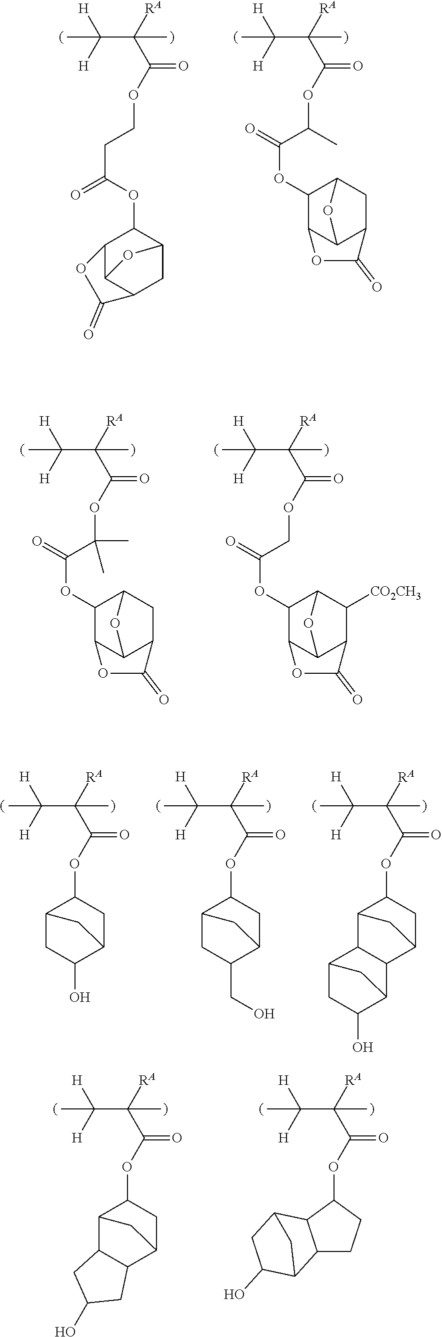

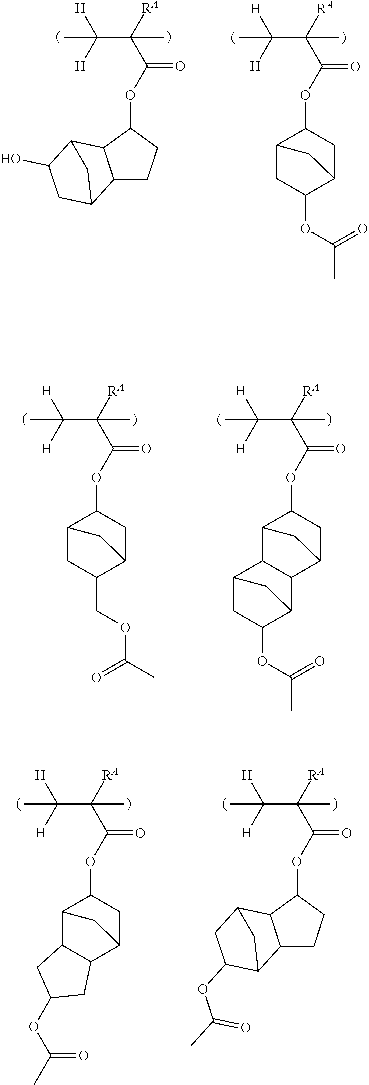

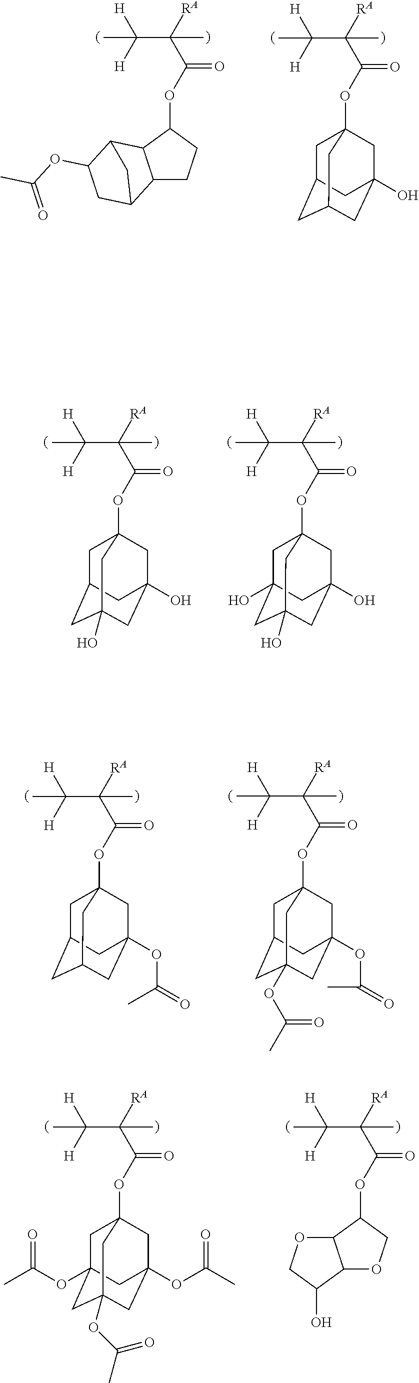

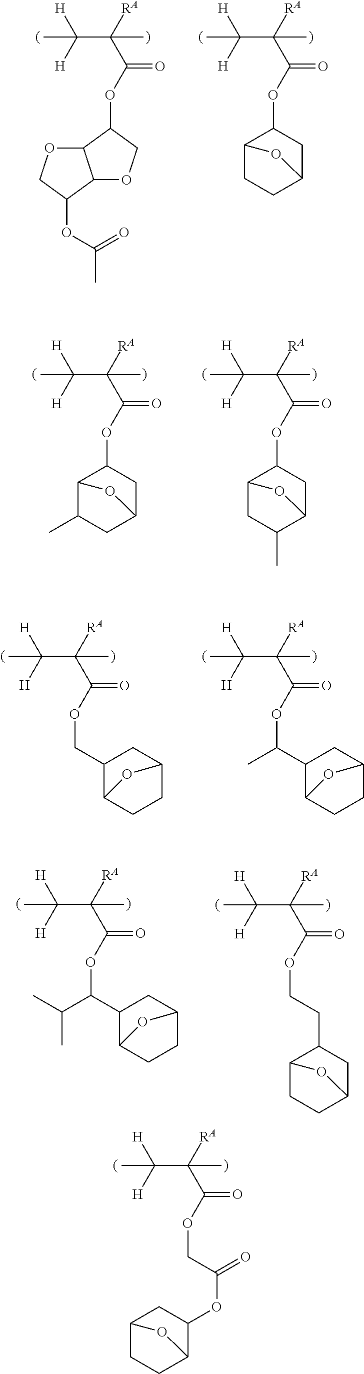

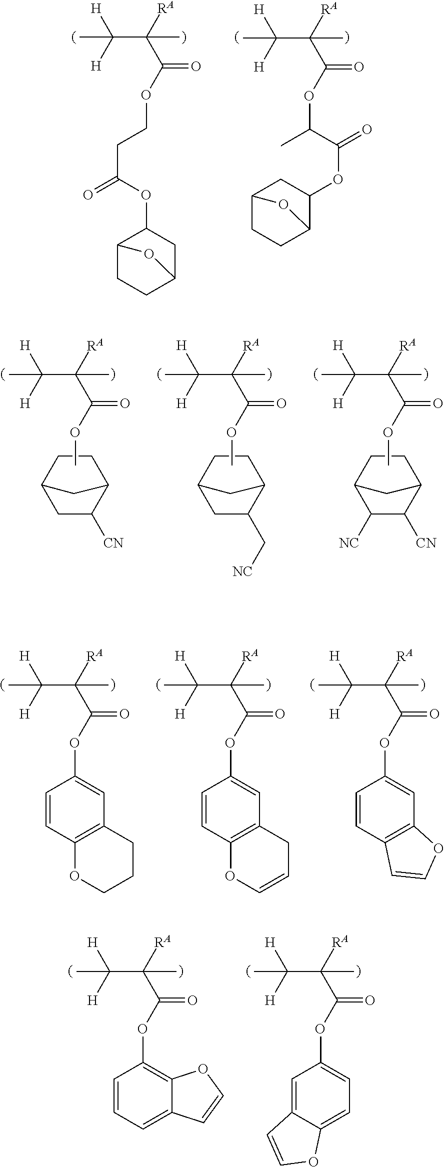

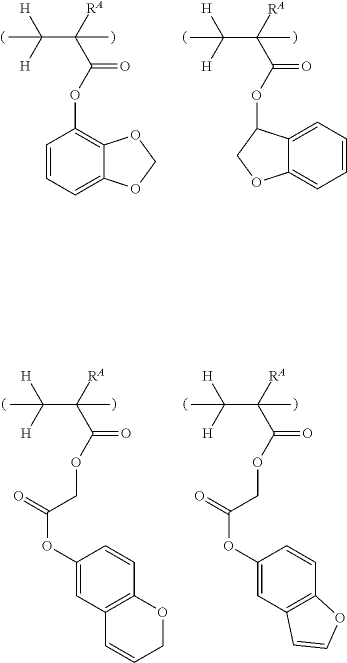

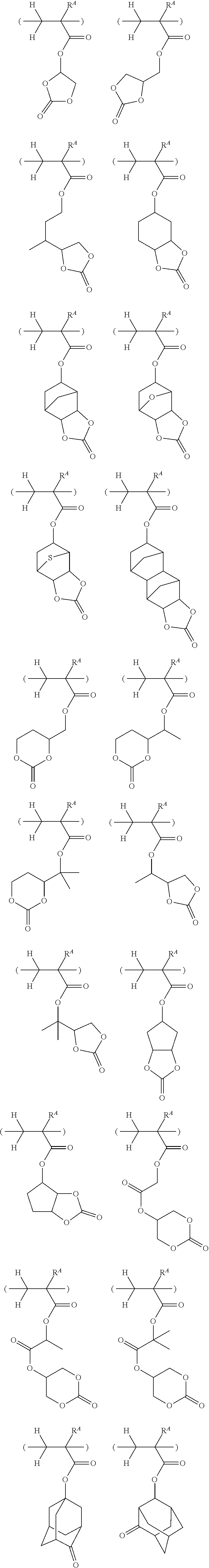

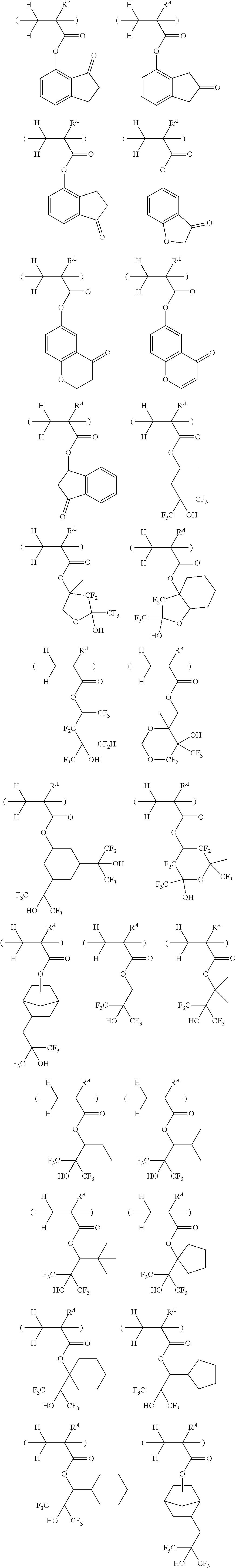

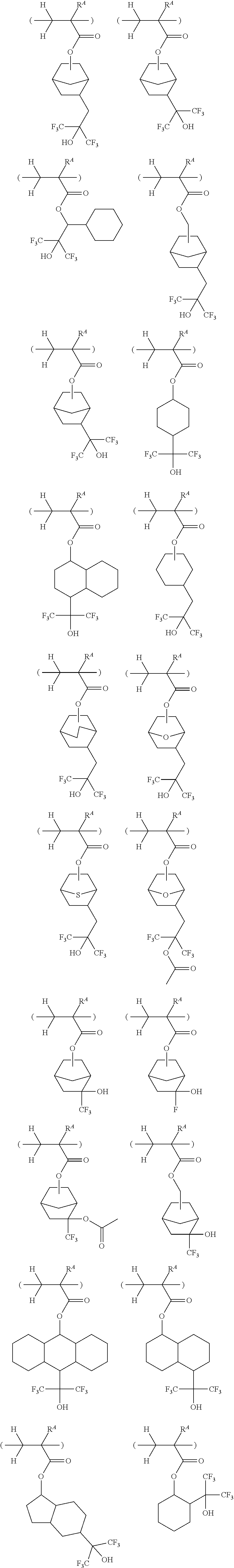

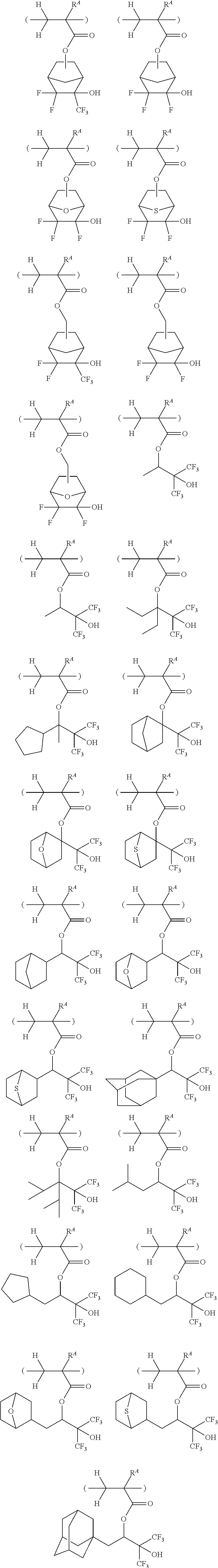

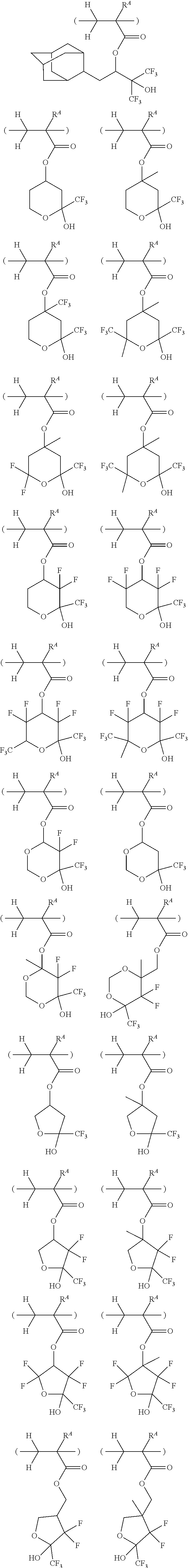

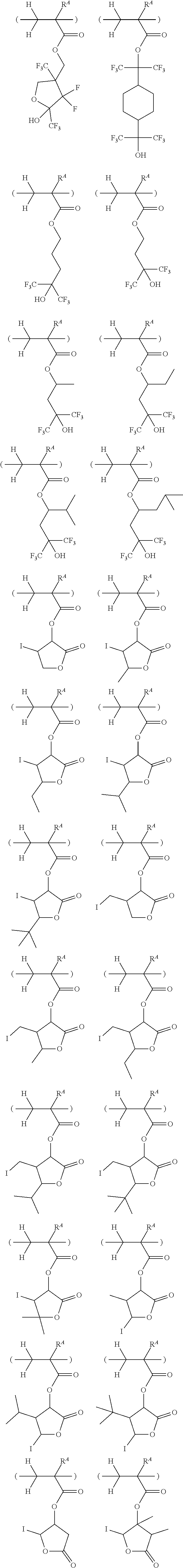

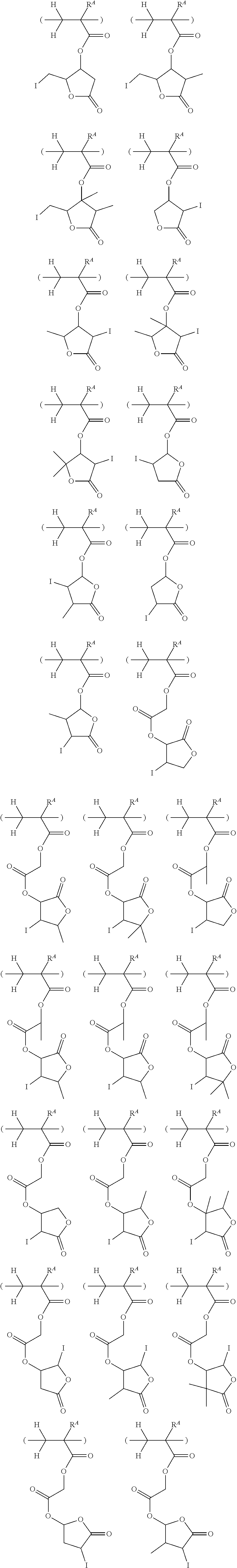

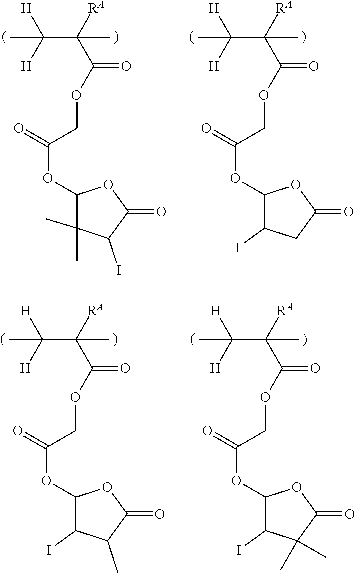

Examples of repeat unit (b2) are shown below, but not limited thereto. Herein R¹ is as defined above.

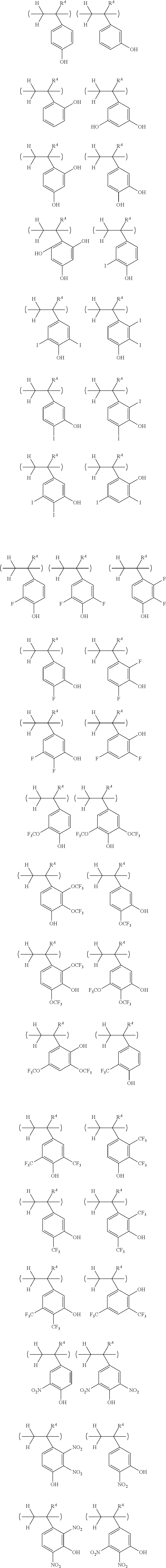

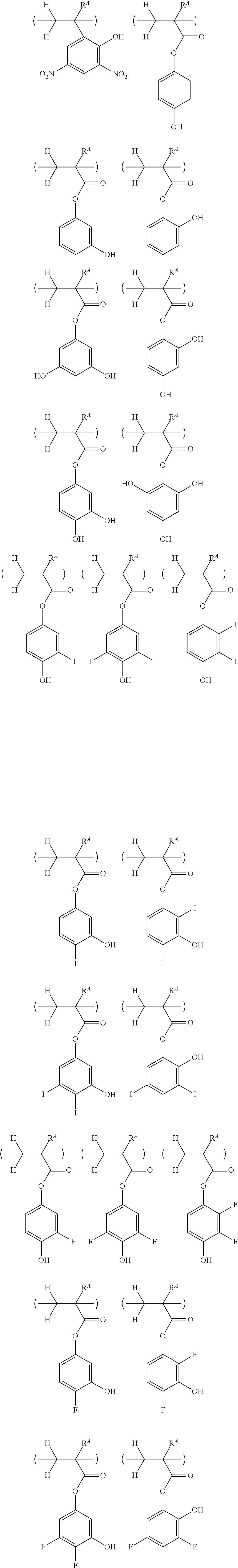

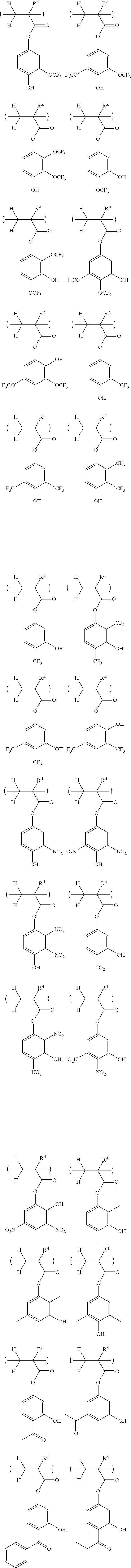

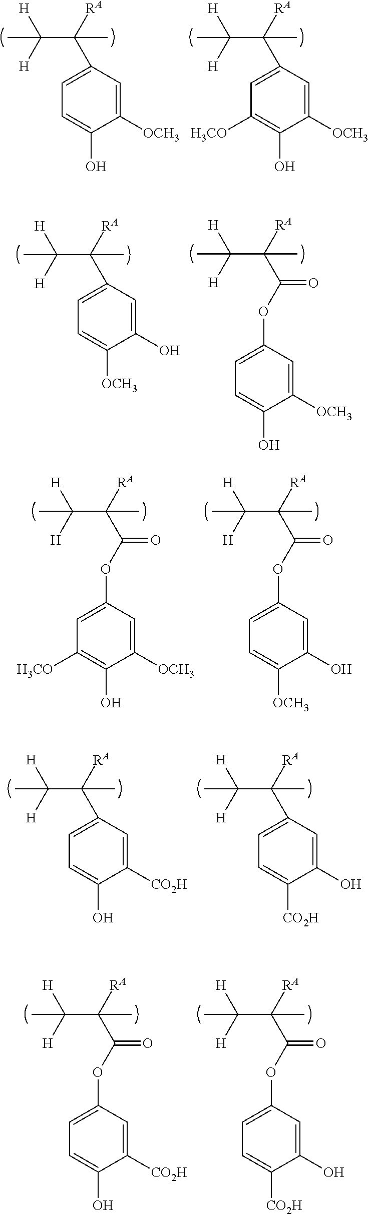

Of the repeat units (b1) and (b2), those units having a lactone ring as the polar group are preferred in the ArF lithography and those units having a phenolic site are preferred in the KrF, EB and EUV lithography.

The base polymer may further comprise repeat units of at least one type selected from repeat units having the formula (c1) (hereinafter, also referred to as repeat units (c1)), repeat units having the formula (c2) (hereinafter, also referred to as repeat units (c2)), repeat units having the formula (c3) (hereinafter, also referred to as repeat units (c3)), repeat units having the formula (c4) (hereinafter, also referred to as repeat units (c4)), and repeat units having the formula (c5) (hereinafter, also referred to as repeat units (c5)).

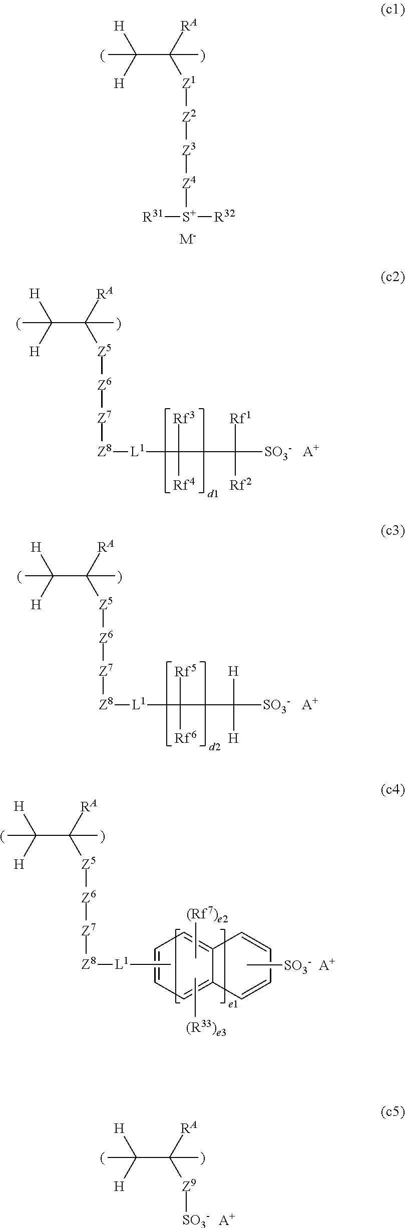

In formulae (c1) to (c4), R^{A} is each independently hydrogen, fluorine, methyl or trifluoromethyl. Z¹ is a single bond or optionally substituted phenylene group. Z² is a single bond, **—C(═O)—O—Z²¹—, **—C(═O)—N(H)—Z²¹— or **—O—Z²¹—. Z²¹ is a C₁-C₆ aliphatic hydrocarbylene group, a phenylene group or a divalent group obtained by combining the foregoing, which may contain halogen, carbonyl moiety, ester bond, ether bond or hydroxy moiety. Z³ is a single bond, ether bond, ester bond, amide bond, sulfonate ester bond, sulfonamide bond, carbonate bond or carbamate bond. Z⁴ is a single bond, or a C₁-C₆ aliphatic hydrocarbylene group, phenylene group or a divalent group obtained by combining the foregoing, which may contain halogen, carbonyl moiety, ester bond, ether bond or hydroxy moiety. Z⁵ is each independently a single bond, optionally substituted phenylene group, naphthylene group, or *—C(═O)—O—Z⁵. Z⁵¹ is a C₁-C₁₀ aliphatic hydrocarbylene group which may contain halogen, hydroxy moiety, ether bond, ester bond or lactone ring, or phenylene or naphthylene group. Z⁶ is a single bond, ether bond, ester bond, amide bond, sulfonate ester bond, sulfonamide bond, carbonate bond or carbamate bond. Z⁷ is each independently a single bond, ***—Z⁷¹—C(═O)—O—, ***—C(═O)—NH—Z⁷—, or ***—O—Z⁷¹—. Z⁷¹ is a C₁-C₂₀ hydrocarbylene group which may contain a heteroatom. Z⁸ is each independently a single bond, ****—Z⁸¹—C(═O)—O—, ****—C(═O)—NH—Z⁸¹—, or ****—O—Z⁸¹—. Z⁸¹ is a C₁-C₂₀ hydrocarbylene group which may contain a heteroatom. Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹—, or *—O—Z⁹—. Z⁹¹ is a C₁-C₆ aliphatic hydrocarbylene group, phenylene group, fluorinated phenylene group or trifluoromethyl substituted phenylene group, which may contain a carbonyl moiety, ester bond, ether bond or hydroxy moiety. The asterisk (*) designates a point of attachment to the carbon atom in the backbone. ** designates a point of attachment to Z¹, *** designates a point of attachment to Z⁶, and **** designates a point of attachment to Z⁷.

The aliphatic hydrocarbylene groups Z²¹, Z⁵¹ and Z⁹¹ may be straight, branched or cyclic. Examples thereof include alkanediyl groups such as methanediyl, ethane-1,1-diyl, ethane-1,2-diyl, propane-1,1-diyl, propane-1,2-diyl, propane-1,3-diyl, propane-2,2-diyl, butane-1,1-diyl, butane-1,2-diyl, butane-1,3-diyl, butane-2,3-diyl, butane-1,4-diyl, 1,1-dimethylethane-1,2-diyl, pentane-1,5-diyl, 2-methylbutane-1,2-diyl, and hexane-1,6-diyl; cycloalkanediyl groups such as cyclopropanediyl, cyclobutanediyl, cyclopentanediyl and cyclohexanediyl, and combinations thereof.

The hydrocarbylene groups Z⁷¹ and Z⁸¹ which may contain a heteroatom may be saturated or unsaturated and straight, branched or cyclic. Examples thereof are shown below, but not limited thereto.

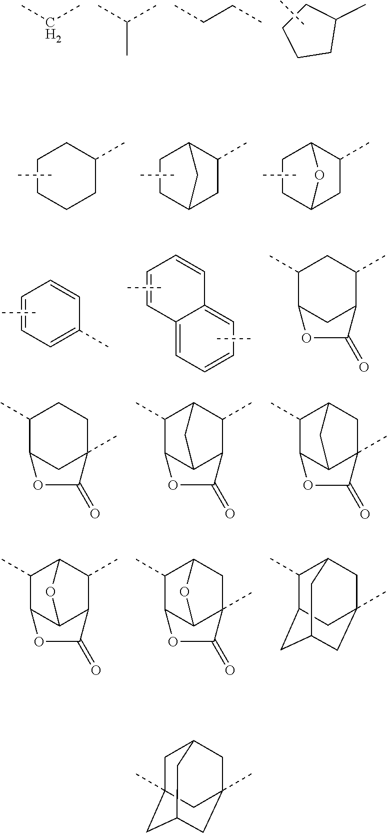

Herein the broken line denotes a point of attachment.

In formula (c1), R³¹ and R³² are each independently a C₁-C₂₀ hydrocarbyl group which may contain a heteroatom. The hydrocarbyl group may be saturated or unsaturated, and may be straight, branched, or cyclic. Examples thereof include C₁-C₂₀ alkyl groups such as methyl, ethyl, n-propyl, isopropyl, n-butyl, sec-butyl and tert-butyl groups; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl and adamantyl groups; C₂-C₂₀ alkenyl groups such as vinyl, 1-propenyl, 2-propenyl, butenyl and hexenyl groups; C₃-C₂₀ cyclic unsaturated hydrocarbyl groups such as a cyclohexenyl group; C₆-C₂₀ aryl groups such as phenyl, naphthyl and thienyl groups; C₇-C₂₀ aralkyl groups such as benzyl, 1-phenylethyl and 2-phenylethyl groups; and combinations thereof. The aryl groups are preferred. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

R³¹ and R¹¹ may bond together to form a ring with the sulfur atom to which they are attached. Examples of the ring are shown below.

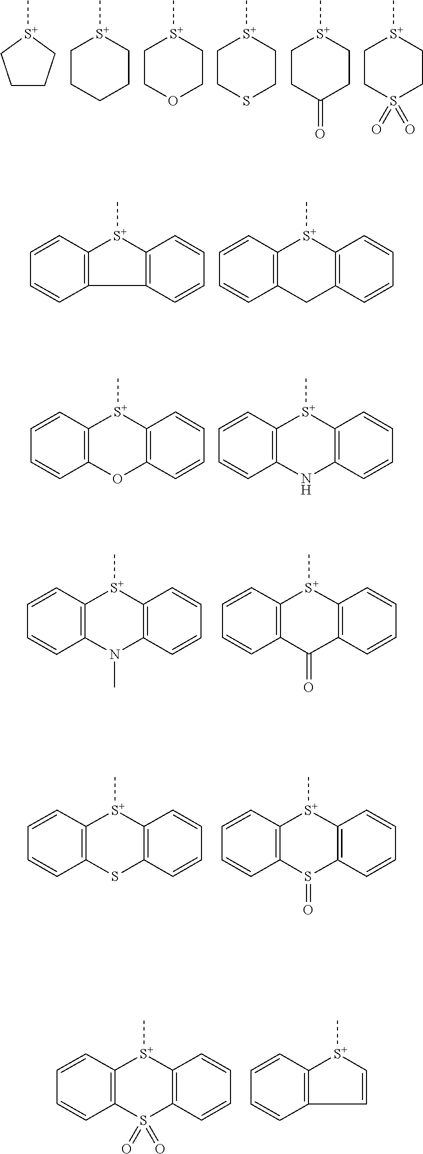

Herein the broken line denotes a point of attachment to Z⁴.

Examples of the cation in repeat unit (c1) are given below, but not limited thereto. Herein R^{A} is as defined above.

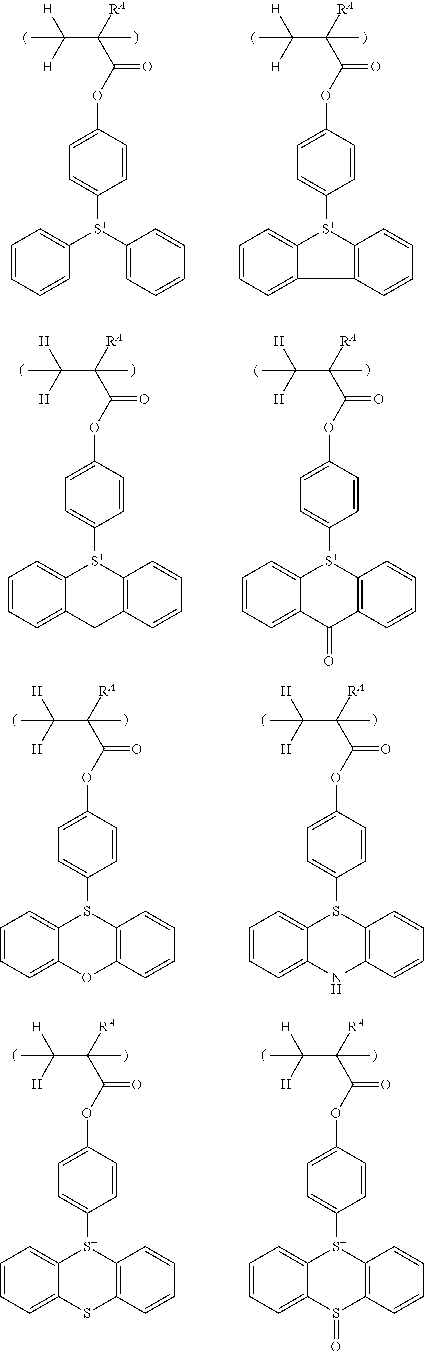

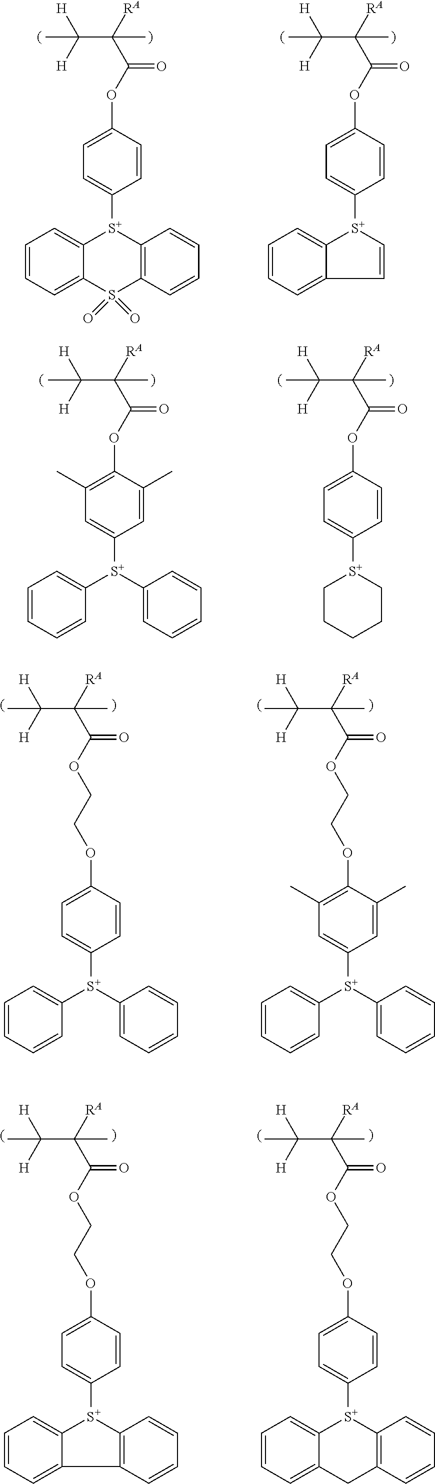

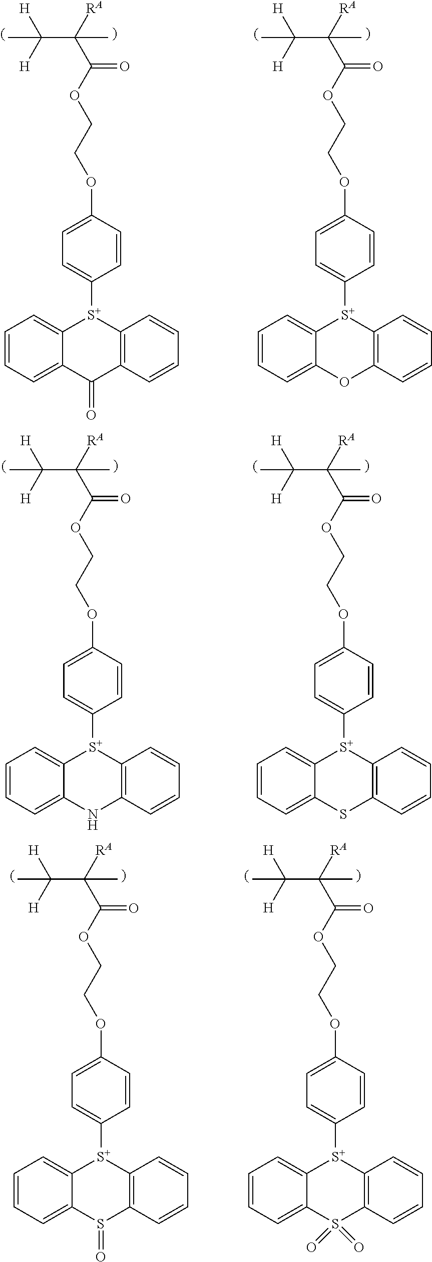

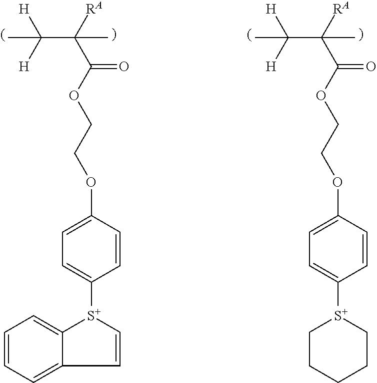

In formula (c1), M⁻ is a non-nucleophilic counter ion. Halide, sulfonate, imide and methide anions are preferred. Examples of the non-nucleophilic counter ion include halide ions such as chloride and bromide ions; sulfonate anions, specifically fluoroalkylsulfonate ions such as triflate, 1,1,1-trifluoroethanesulfonate, and nonafluorobutanesulfonate, arylsulfonate ions such as tosylate, benzenesulfonate, 4-fluorobenzenesulfonate, and 1,2,3,4,5-pentafluorobenzenesulfonate, alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; and methide ions such as tris(trifluoromethylsulfonyl)methide and tris(perfluoroethylsulfonyl)methide.

Anions having the following formulae (c1-1) to (c1-4) are also useful as the non nucleophilic counter ion.

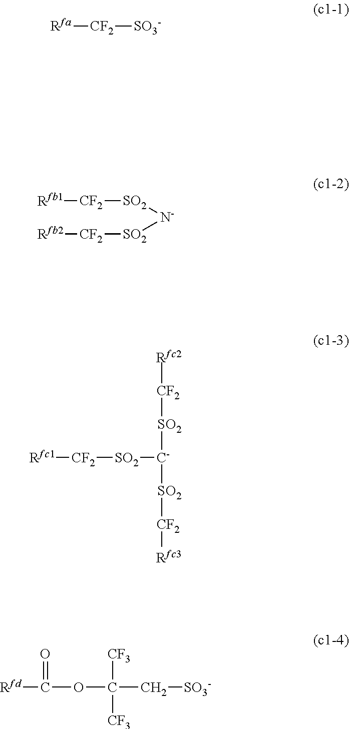

In formula (c1-1), R^{fa} is fluorine or a C₁-C₄₀ hydrocarbyl group which may contain a heteroatom. The hydrocarbyl group may be saturated or unsaturated, and may be straight, branched, or cyclic. Examples thereof are as will be exemplified below for the hydrocarbyl group R^{fa1} in formula (c1-1-1).

Of the anions of formula (c1-1), an anion having the formula (c1-1-1) is preferred.

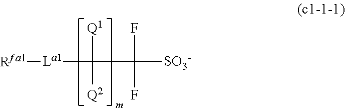

In formula (c1-1-1), Q¹ and Q² are each independently hydrogen, fluorine or a C₁-C₆ fluorinated saturated hydrocarbyl group. It is preferred for solvent solubility that at least one of Q¹ and Q² be trifluoromethyl. The subscript m is 0, 1, 2, 3 or 4, preferably 1. R^{fa1} is a C₁-C₃₅ hydrocarbyl group which may contain a heteroatom. As the heteroatom, oxygen, nitrogen, sulfur and halogen atoms are preferred, with oxygen being most preferred. Of the hydrocarbyl groups, those groups of 6 to 30 carbon atoms are preferred from the aspect of achieving a high resolution in forming patterns of small feature size.

In formula (c1-1-1), the C₁-C₃₅ hydrocarbyl group R^{fa1} may be saturated or unsaturated and straight, branched or cyclic. Examples thereof include C₁-C₃₅ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl and icocyl groups; C₃-C₃₅ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclodecyl, tetracyclodecylmethyl and dicyclohexylmethyl groups; C₂-C₃₅ unsaturated aliphatic hydrocarbyl groups such as 2-propenyl and 3-cyclohexenyl groups; C₆-C₃₅ aryl groups such as phenyl, 1-naphthyl, 2-naphthyl and 9-fluorenyl groups; C₇-C₃₅ aralkyl groups such as benzyl and diphenylmethyl groups; and combinations thereof.

In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl groups.

In formula (c1-1-1), L¹ is a single bond, ether bond, ester bond or sulfonic ester bond. From the aspect of synthesis, an ether bond or ester bond is preferred, with the ester bond being more preferred.

Examples of the anion having formula (c1-1) are shown below, but not limited thereto. In the following formulae, Q¹ is as defined above, and Ac is an acetyl group.

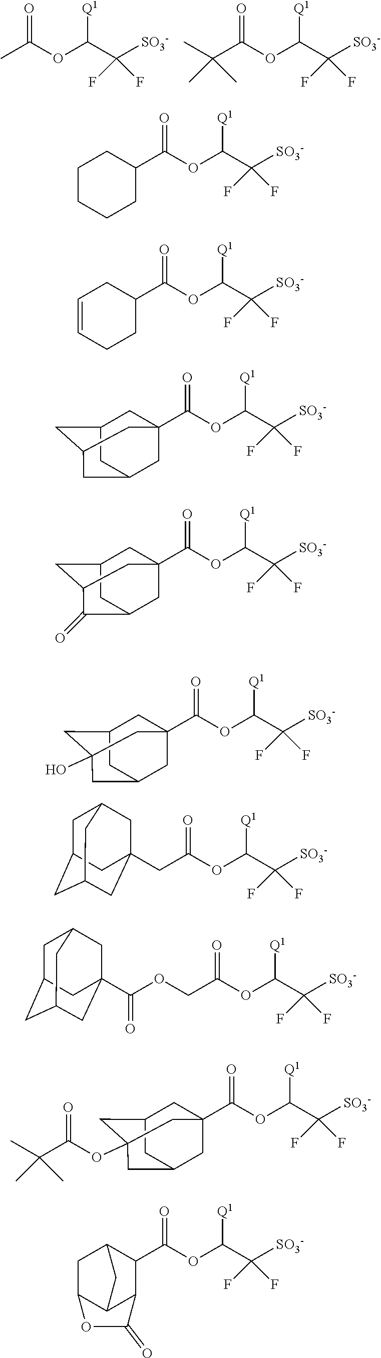

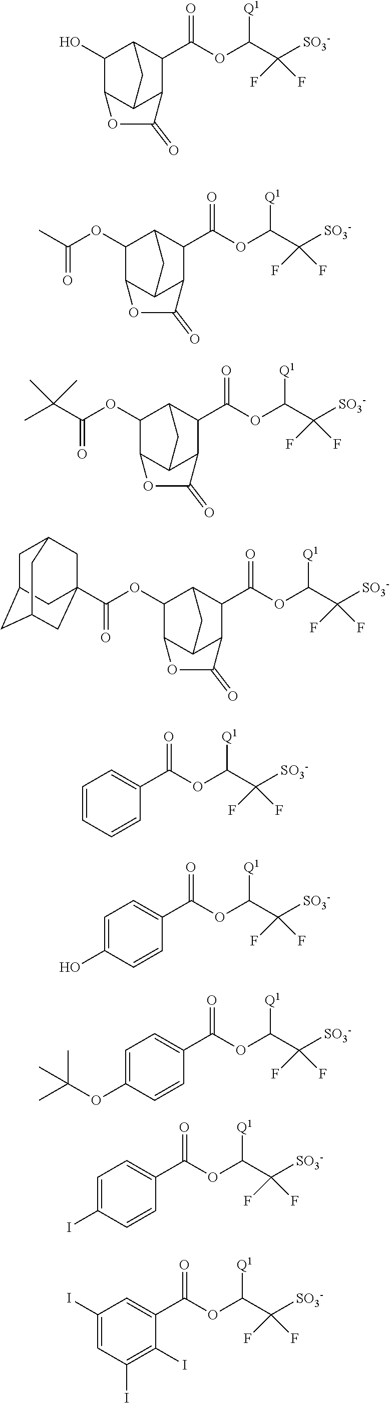

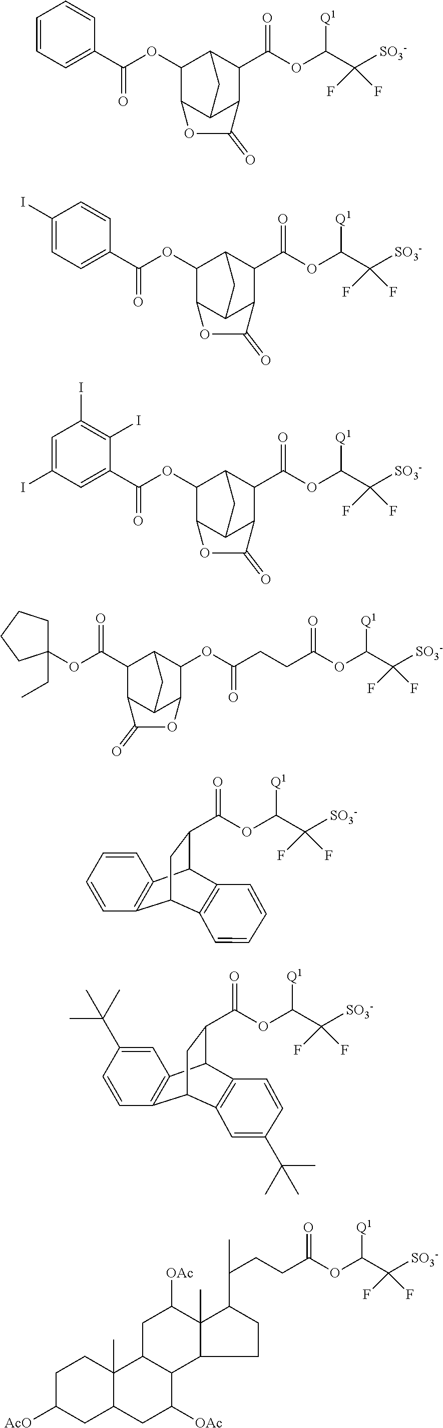

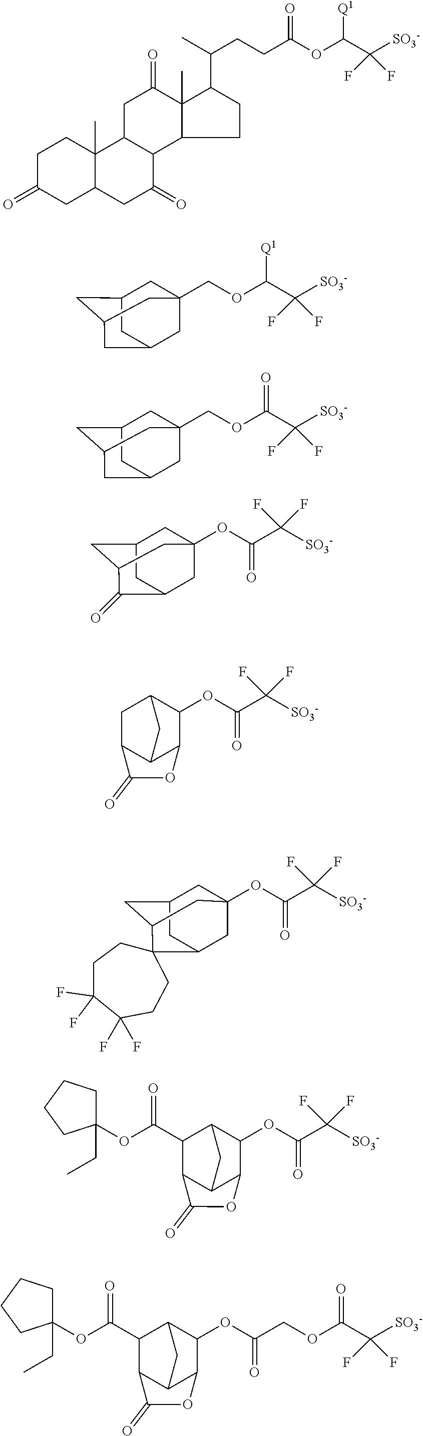

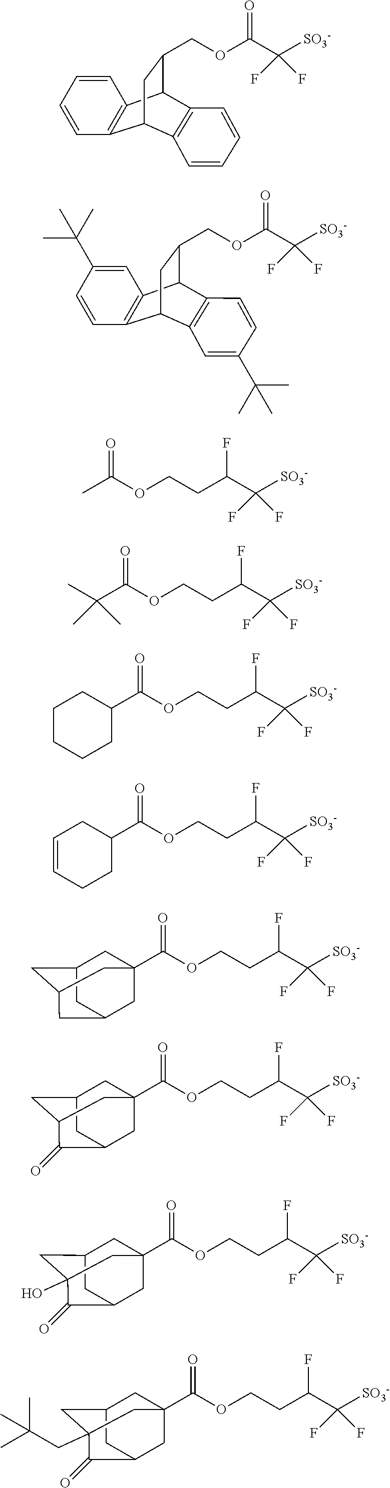

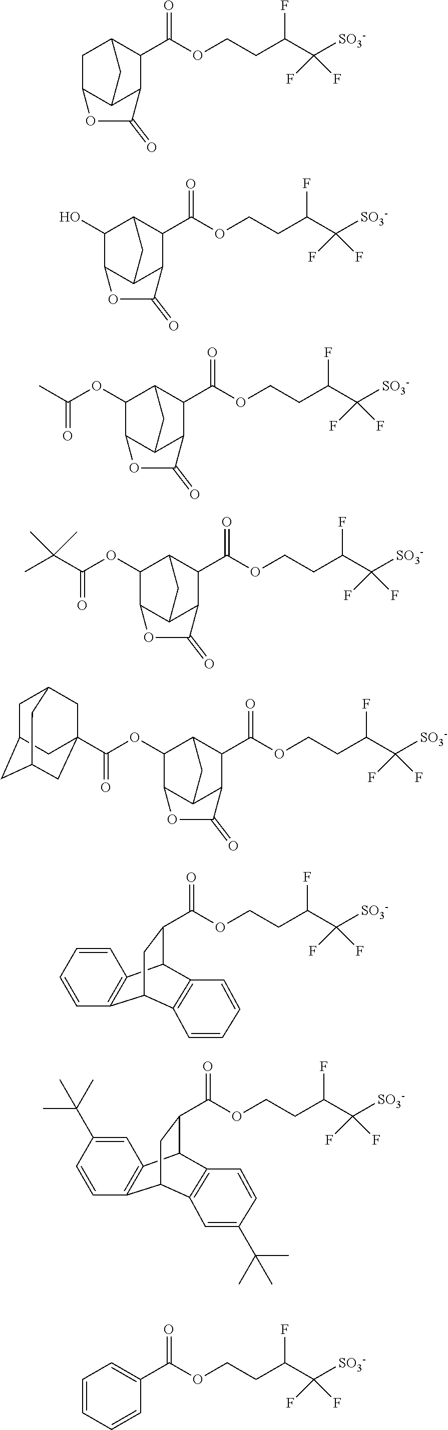

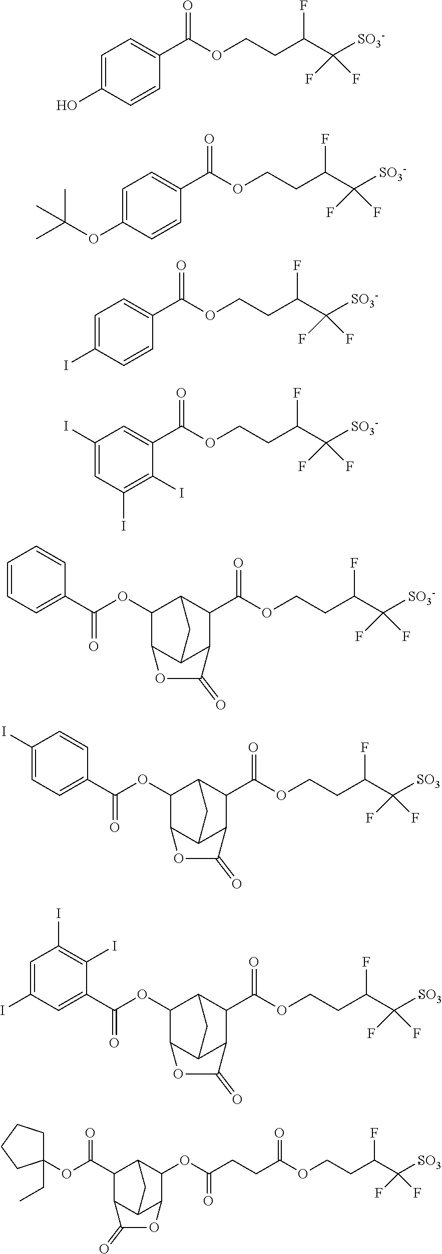

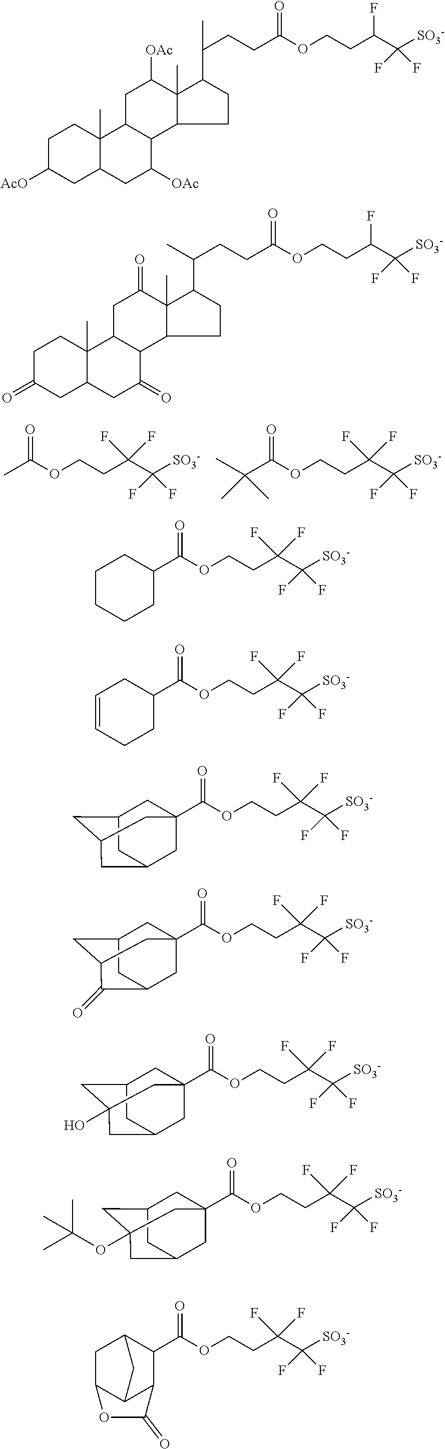

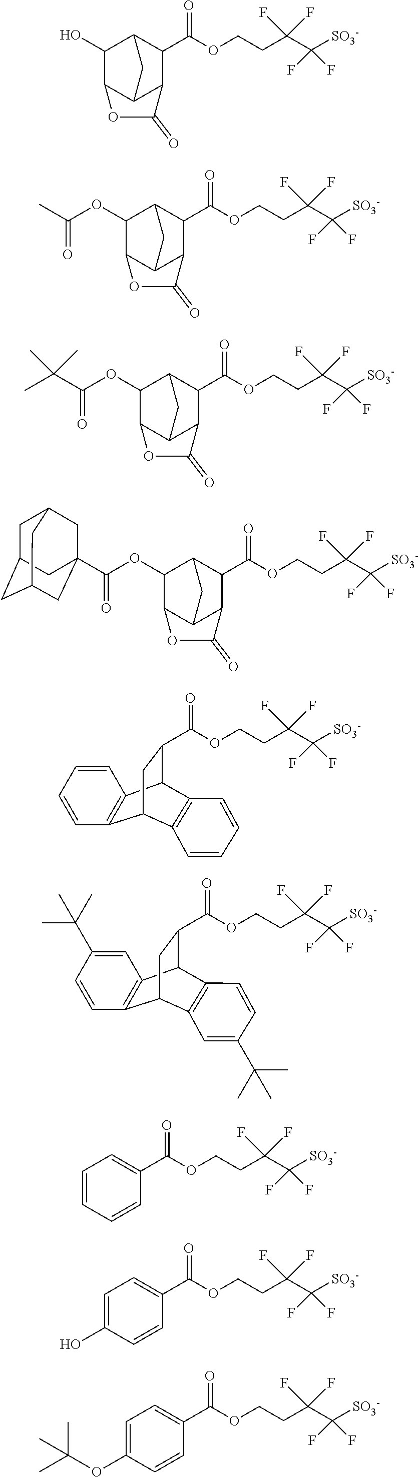

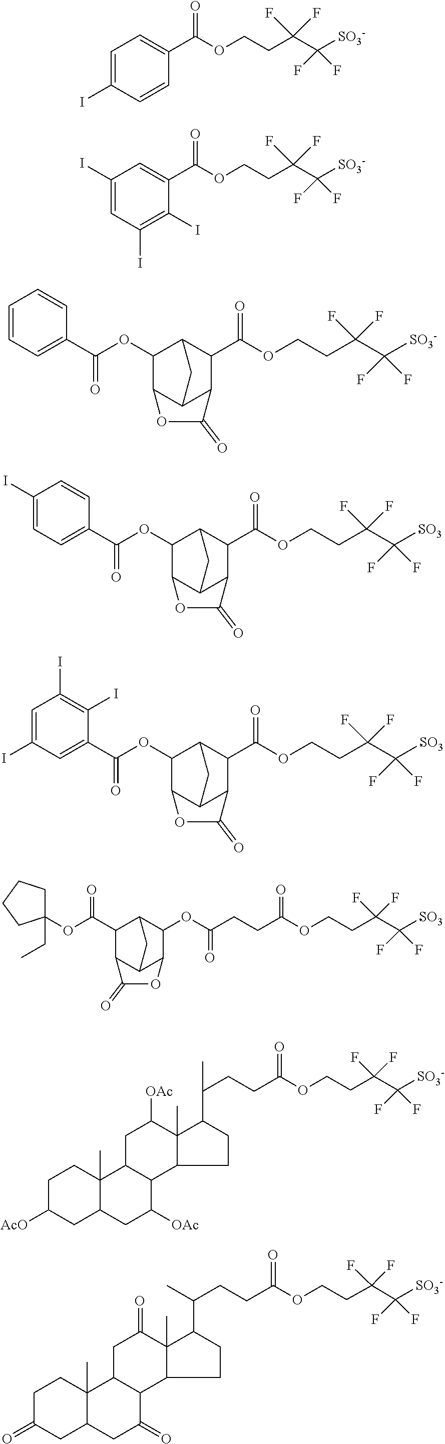

In formula (c1-2), R^{fb1} and R^{fb2} are each independently fluorine, or a C₁-C₄₀ hydrocarbyl group which may contain a heteroatom. The hydrocarbyl group may be saturated or unsaturated, and may be straight, branched, or cyclic. Examples thereof are as exemplified above for the hydrocarbyl group R^{fa1} in formula (c1-1-1). Preferably R^{fb1} and R^{fb2} are fluorine or C₁-C₄ straight fluorinated alkyl groups. Also, R^{fb1} and R^{fb2} may bond together to form a ring with the linkage: —CF₂—SO₂—N⁻—SO₂—CF₂— to which they are attached. It is preferred that a combination of R^{fb1} and R^{fb2} be a fluorinated ethylene or fluorinated propylene group.

In formula (c1-3), R^{fc1}, R^{fc2} and R^{fc3} are each independently fluorine or a C₁-C₄₀ hydrocarbyl group which may contain a heteroatom. The hydrocarbyl group may be saturated or unsaturated, and may be straight, branched, or cyclic. Examples thereof are as exemplified above for the hydrocarbyl group R^{fa1} in formula (c1-1-1). Preferably R^{fc1}, R^{fc2} and R^{fc3} are fluorine or C₁-C₄ straight fluorinated alkyl groups. Also, R^{fc1} and R^{fc2} may bond together to form a ring with the linkage: —CF₂—SO₂—C⁻—SO₂—CF₂— to which they are attached. It is preferred that a combination of R^{fc1} and R^{fc2} be a fluorinated ethylene or fluorinated propylene group.

In formula (c1-4), R^{fd} is a C₁-C₄₀ hydrocarbyl group which may contain a heteroatom. The hydrocarbyl group may be saturated or unsaturated, and may be straight, branched, or cyclic. Examples thereof are as exemplified above for the hydrocarbyl group R^{fa1} in formula (c1-1-1).

Examples of the anion having formula (c1-4) are shown below, but not limited thereto.

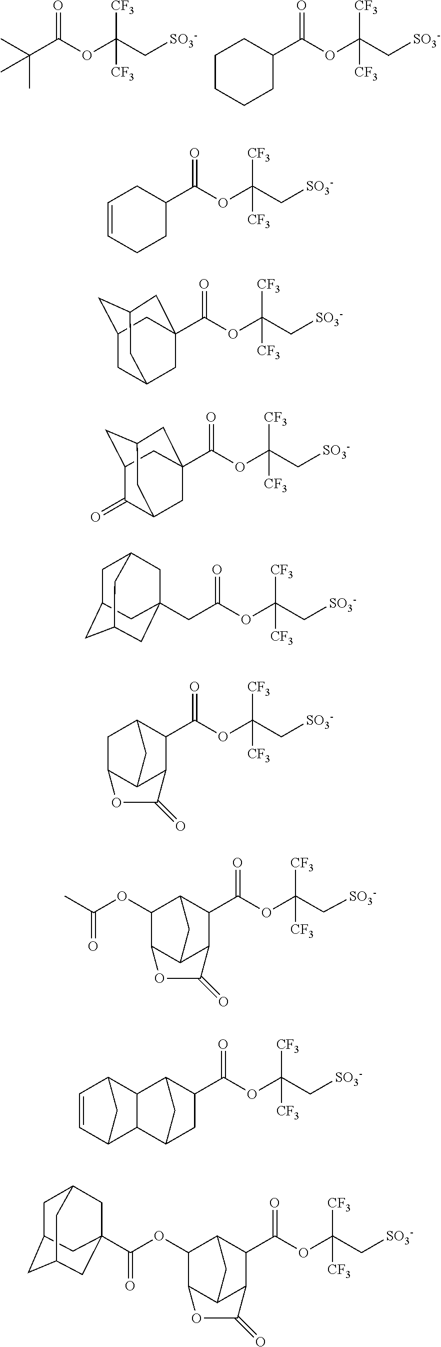

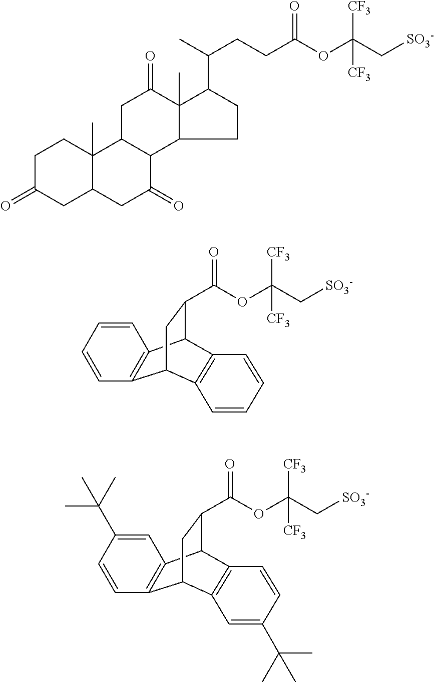

Anions having an iodized or brominated aromatic ring are also useful as the non nucleophilic counter ion. These anions have the formula (c1-5).

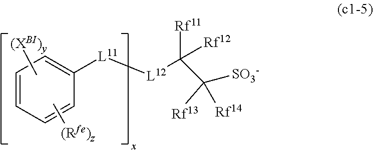

In formula (c1-5), x is 1, 2 or 3. The subscript y is 1, 2, 3, 4 or 5. The subscript z is 0, 1, 2 or 3. It is noted that y+z is from 1 to 5. The subscript y is preferably 1, 2 or 3, more preferably 2 or 3. The subscript z is preferably 0, 1 or 2.

In formula (c1-5), X^{B1} is iodine or bromine, and may be different or identical when x and/or y are 2 or more.

In formula (c1-5), L¹¹ is a single bond, ether bond, ester bond, or a C₁-C₆ saturated hydrocarbylene group which may contain an ether bond or ester bond. The saturated hydrocarbylene group may be straight, branched or cyclic.

In formula (c1-5), L¹² is a single bond or a C₁-C₂₀ divalent linking group when x=1, or a C₁-C₂₀ (x+1) valent linking group when x=2 or 3. The linking group may contain an oxygen, sulfur or nitrogen atom.

In formula (c1-5), R^{fe} is hydroxy, carboxy, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy, or C₁-C₂₀ hydrocarbylsulfonyloxy group, which may contain fluorine, chlorine, bromine, hydroxy, amino or ether bond, or —N(R^{feA})(R^{feB}), —N(R^{feC})—C(═O)—R^{feD} or —N(R^{feC})—C(═O)—O—R^{feD}. R^{feA} and R^{feB} are each independently hydrogen or a C₁-C₆ saturated hydrocarbyl group. R^{feC} is hydrogen, or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{fe}D is a C₁-C₁₆ aliphatic hydrocarbyl group, C₆-C₁₂ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R^{fe} may be identical or different when x and/or z is 2 or more.

Of these, R^{fe} is preferably hydroxy, —N(R^{feC})—C(═O)—R^{feD}, —N(R^{feC})—C(═O)—O—R^{feD}, fluorine, chlorine, bromine, methyl or methoxy.

In formula (c1-5), Rf¹¹ to Rf¹⁴ are each independently hydrogen, fluorine or trifluoromethyl, at least one of R^{f11} to R^{f14} is fluorine or trifluoromethyl. Rf¹¹ and Rf¹², taken together, may form a carbonyl group. More preferably, Rf¹³ and Rf¹⁴ are fluorine.

Examples of the anion having formula (c1-5) are shown below, but not limited thereto. X^{B1} is as defined above.

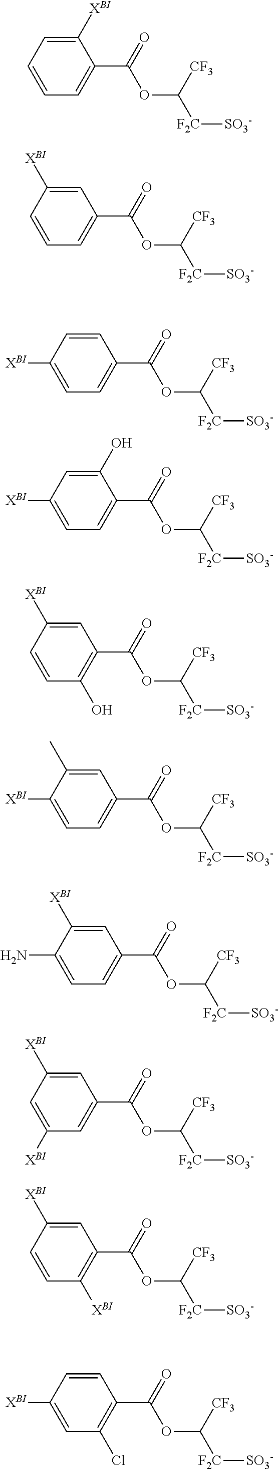

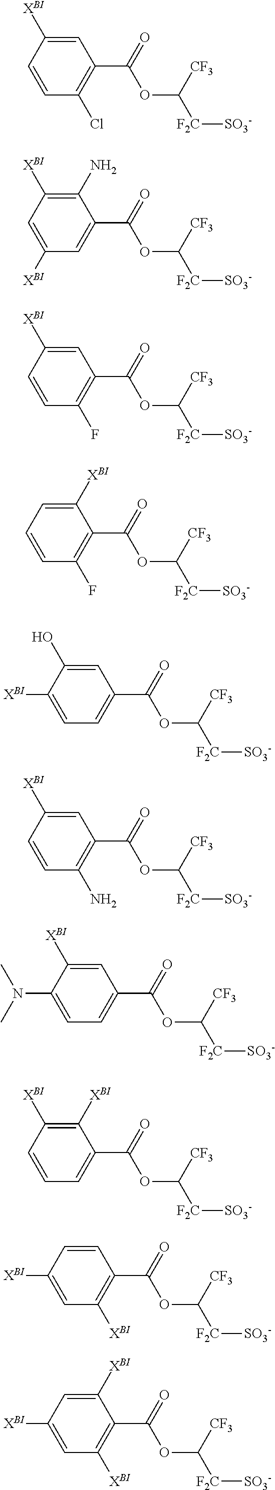

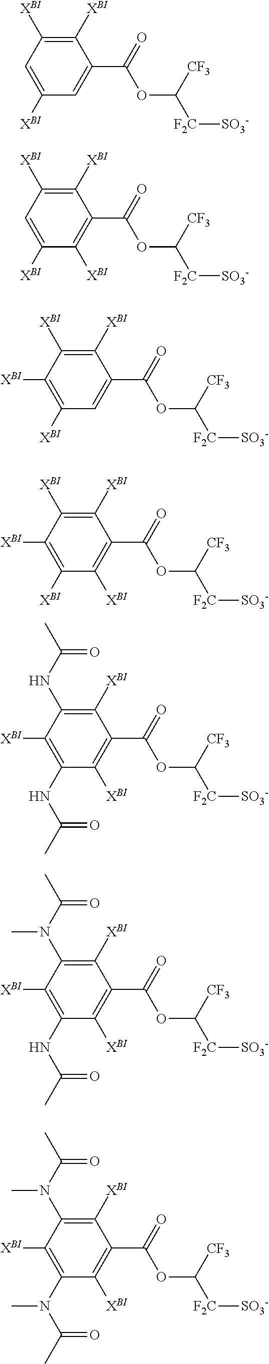

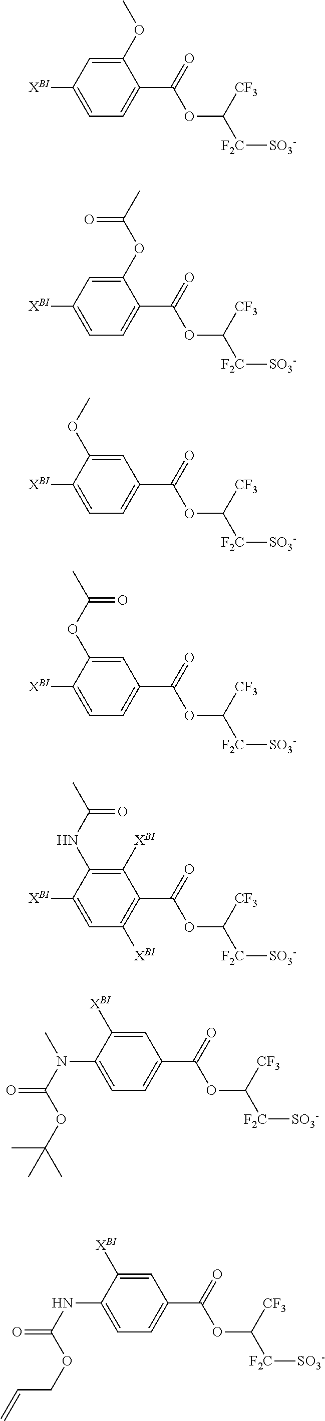

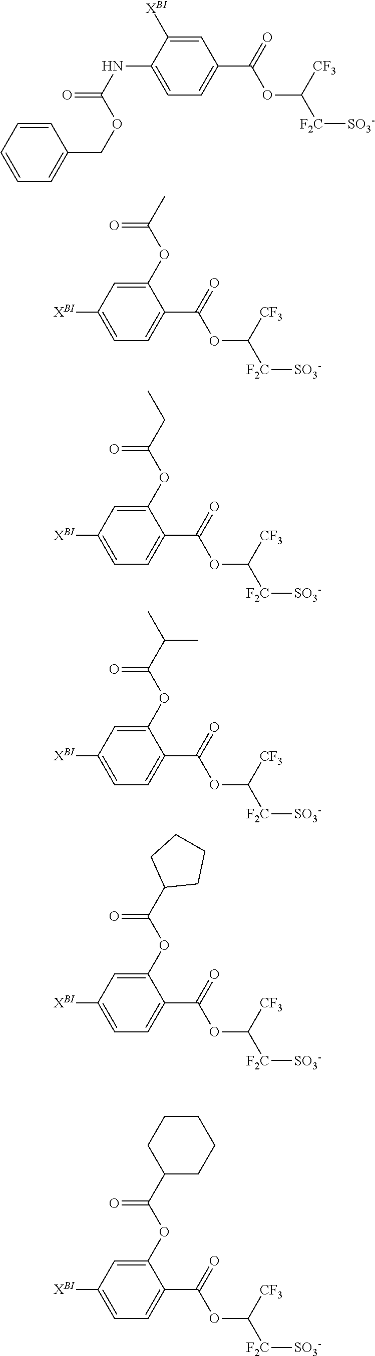

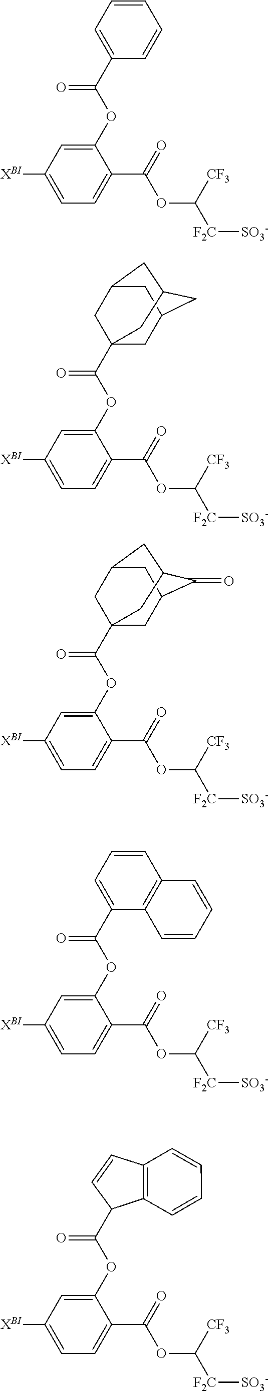

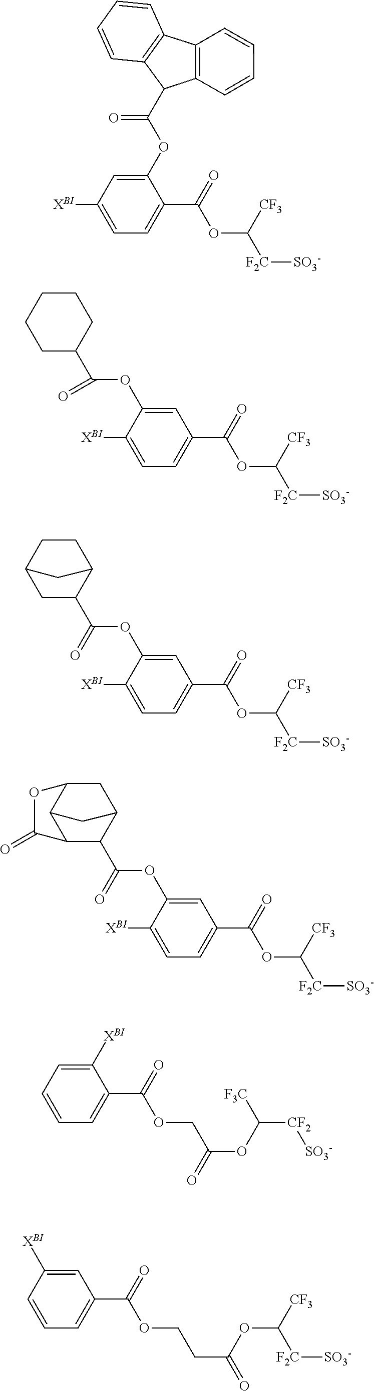

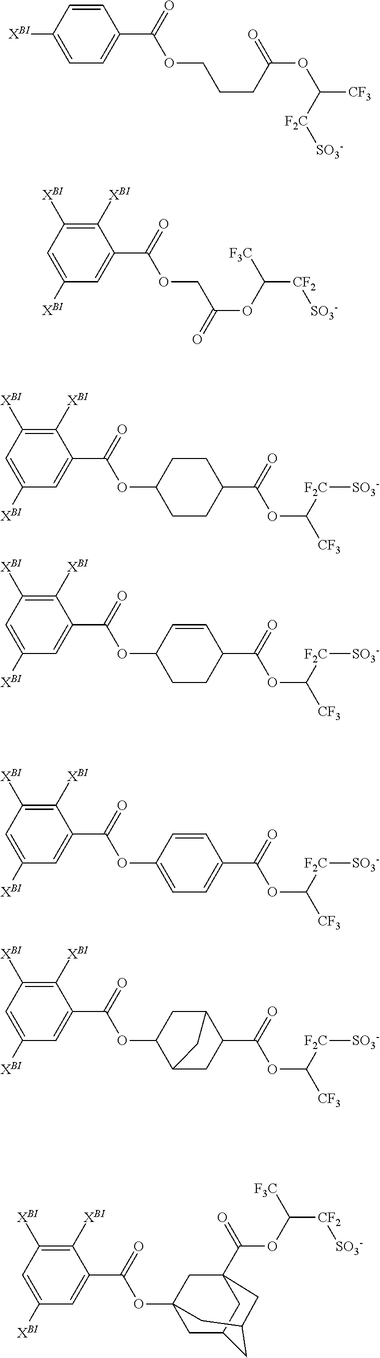

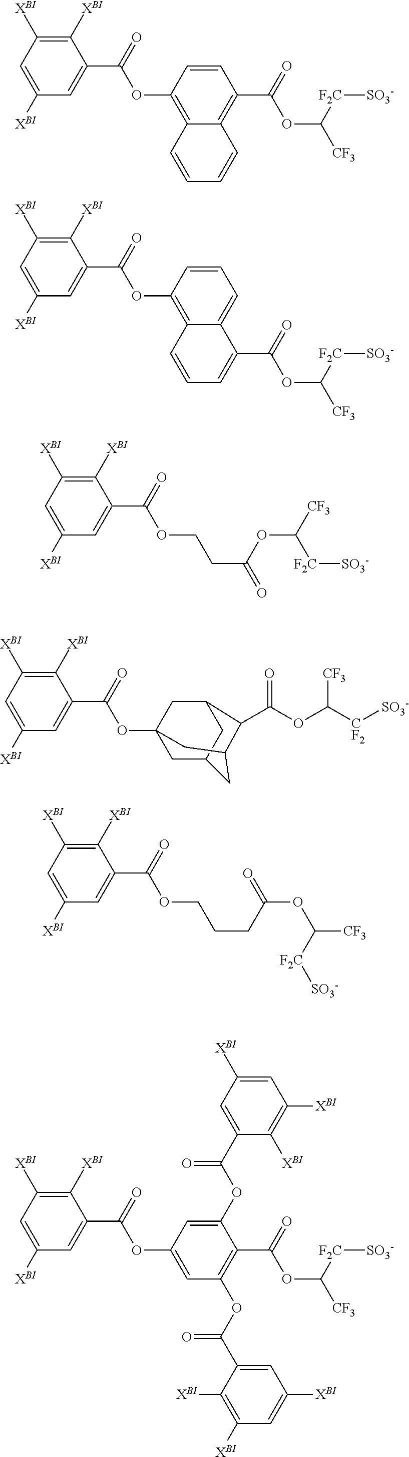

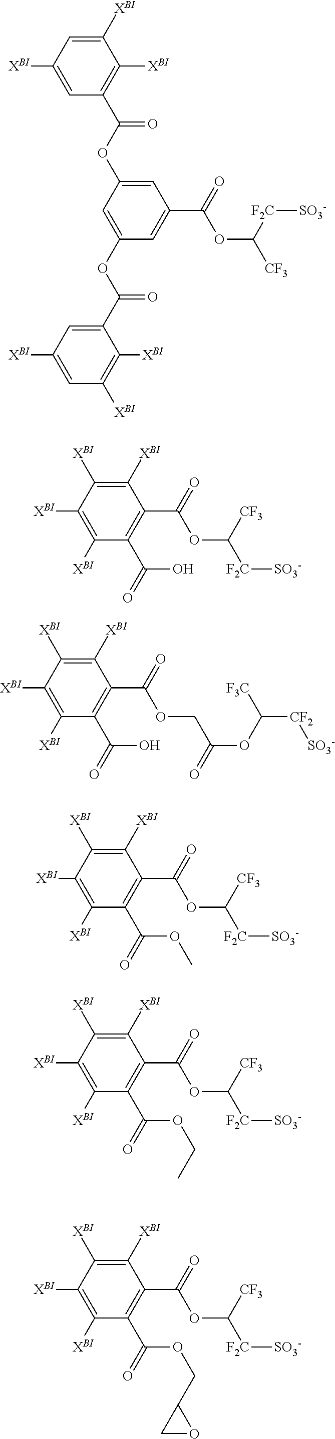

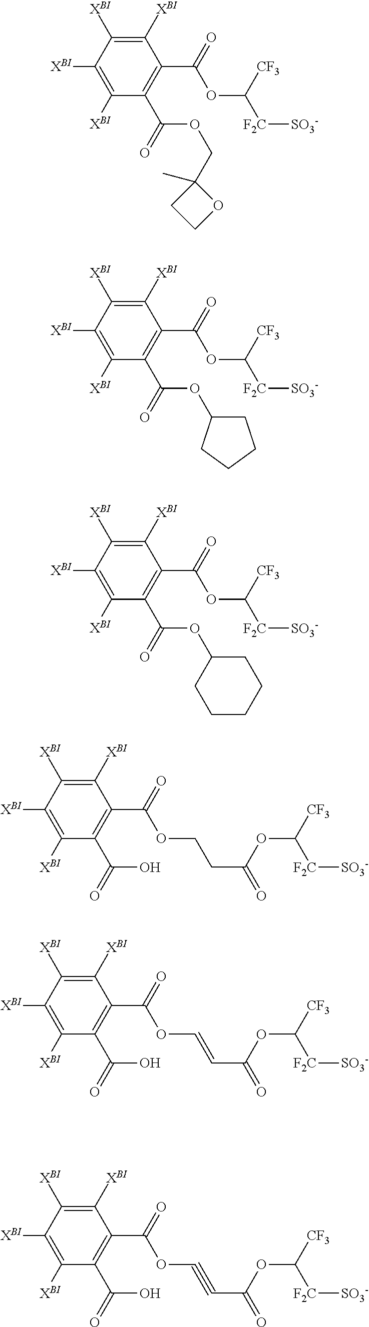

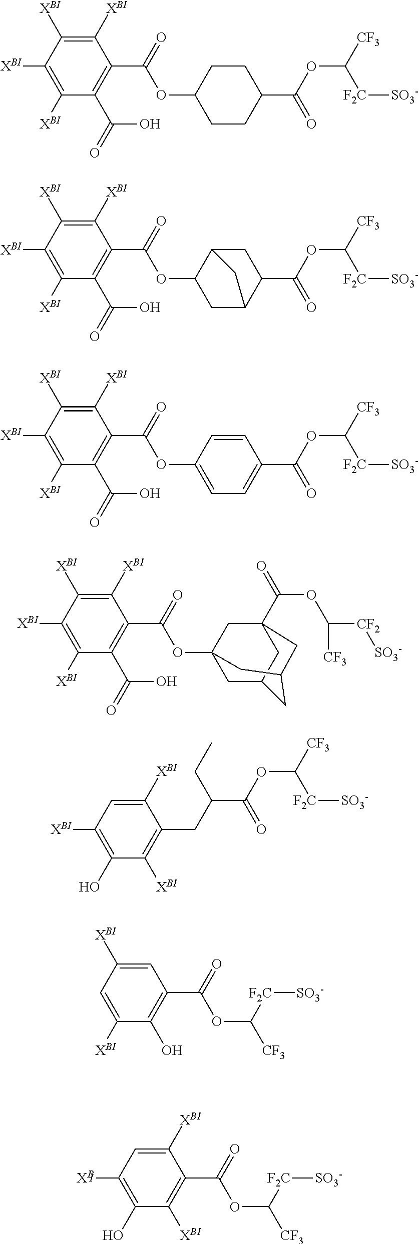

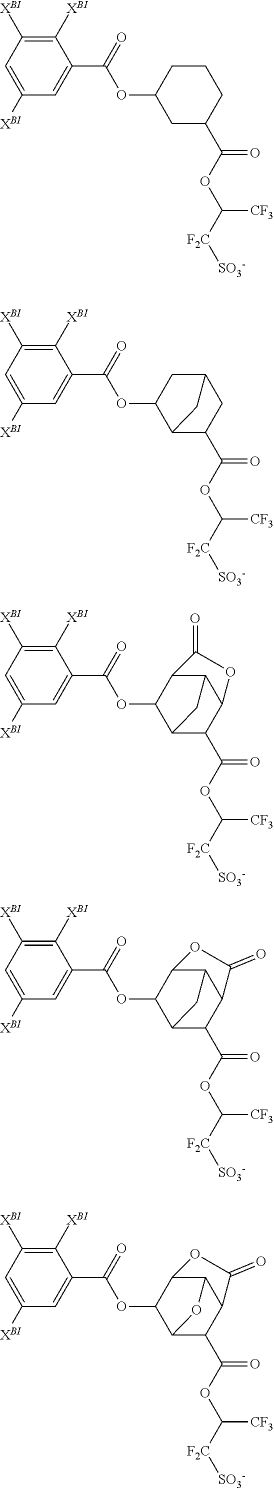

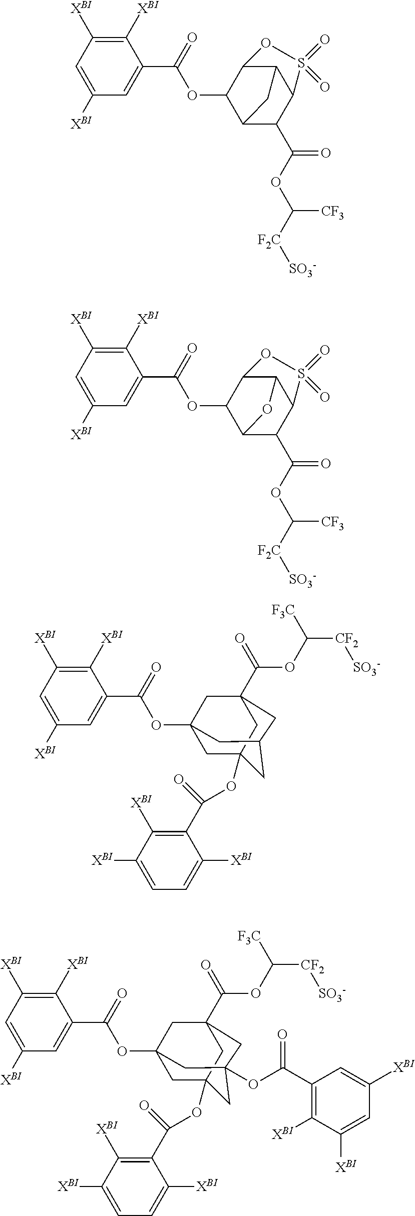

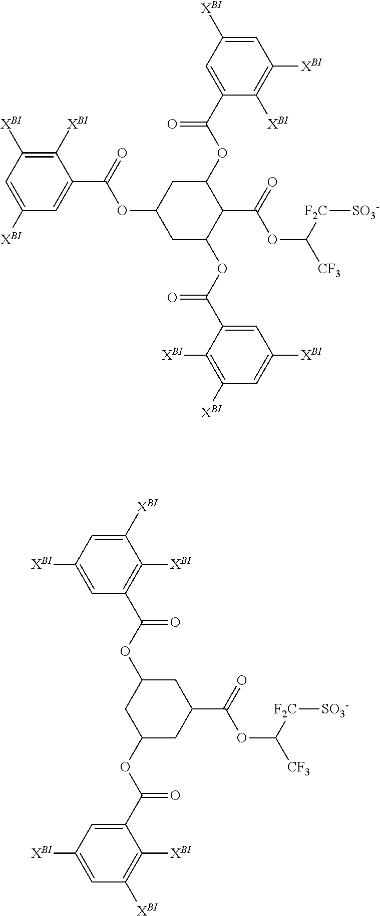

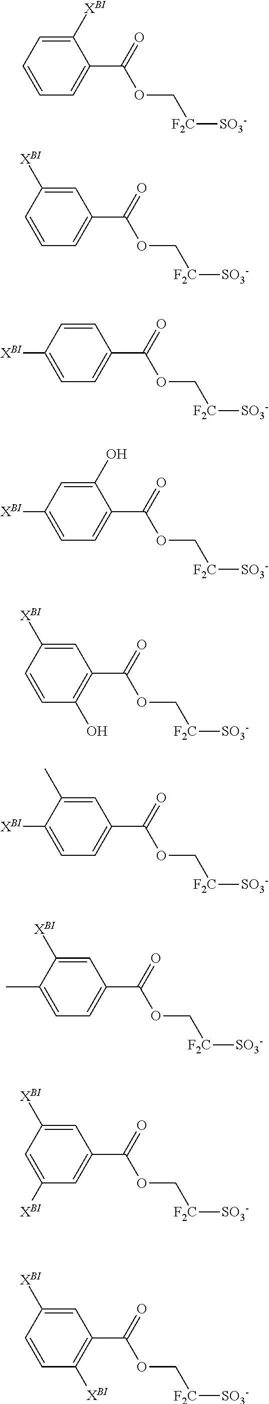

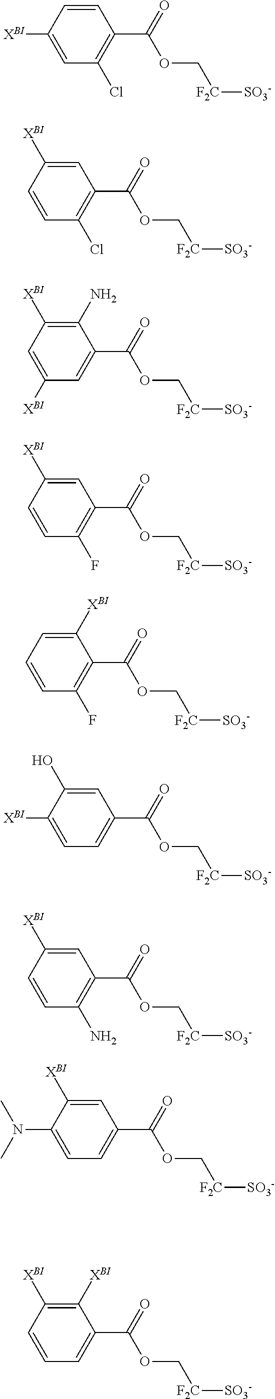

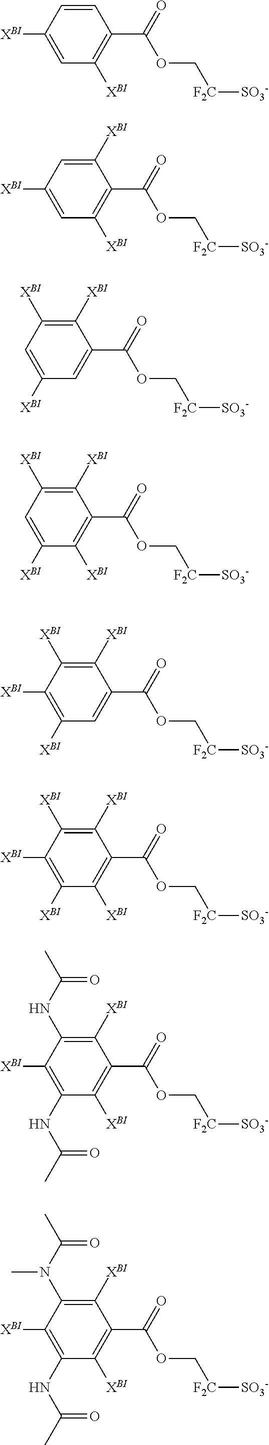

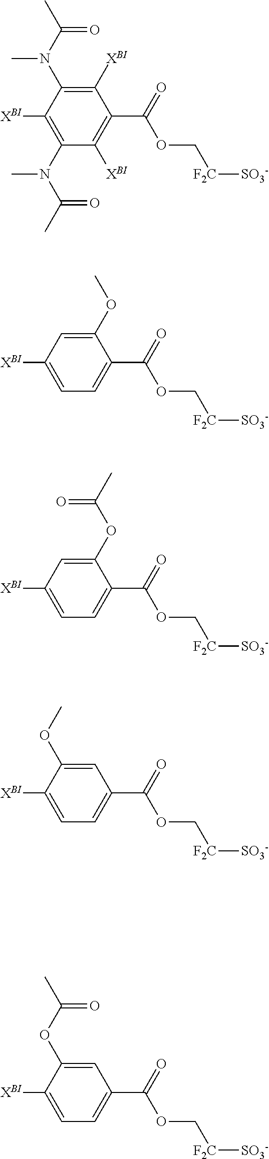

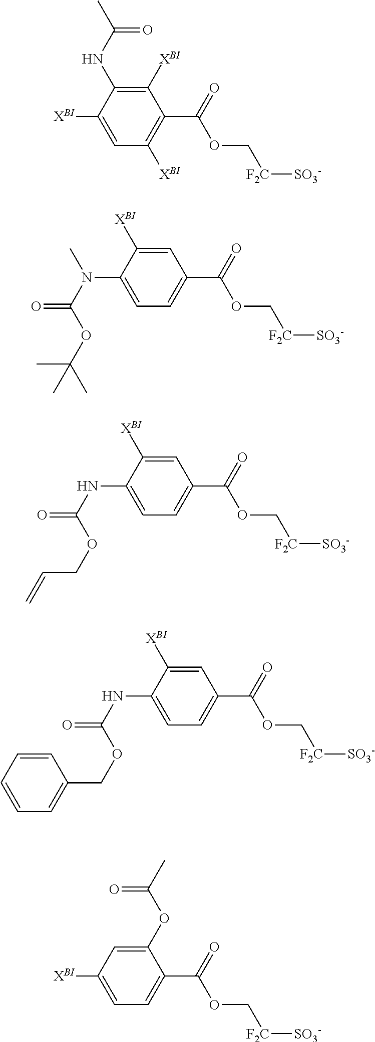

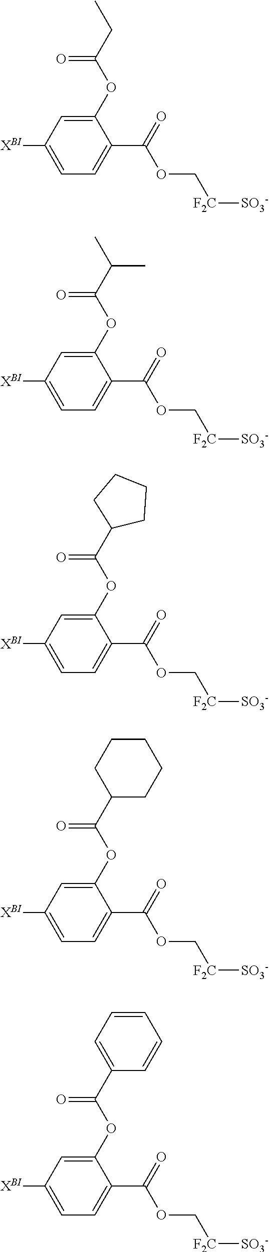

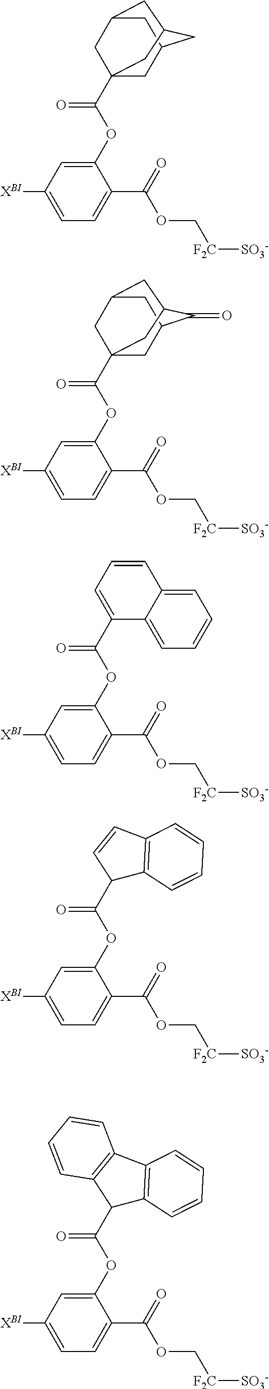

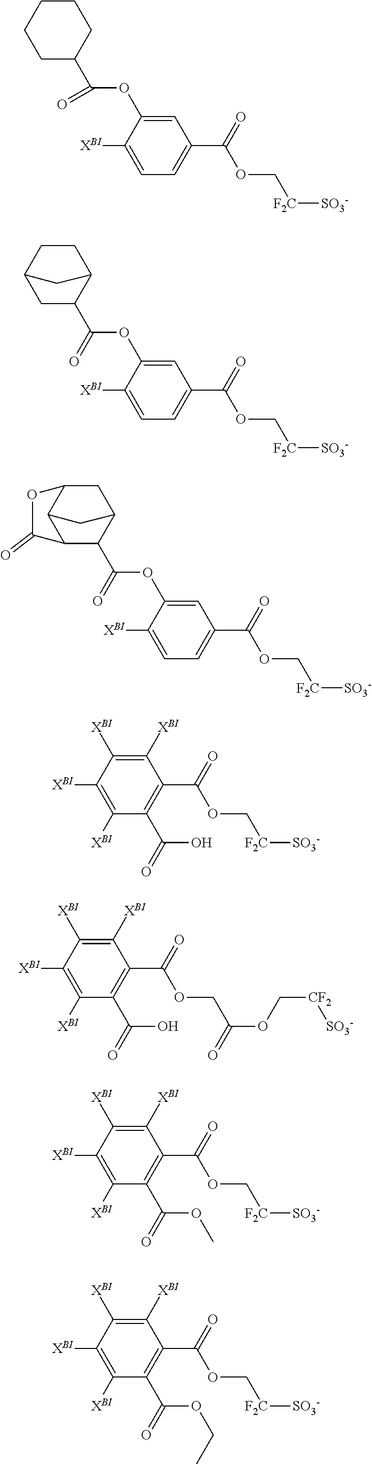

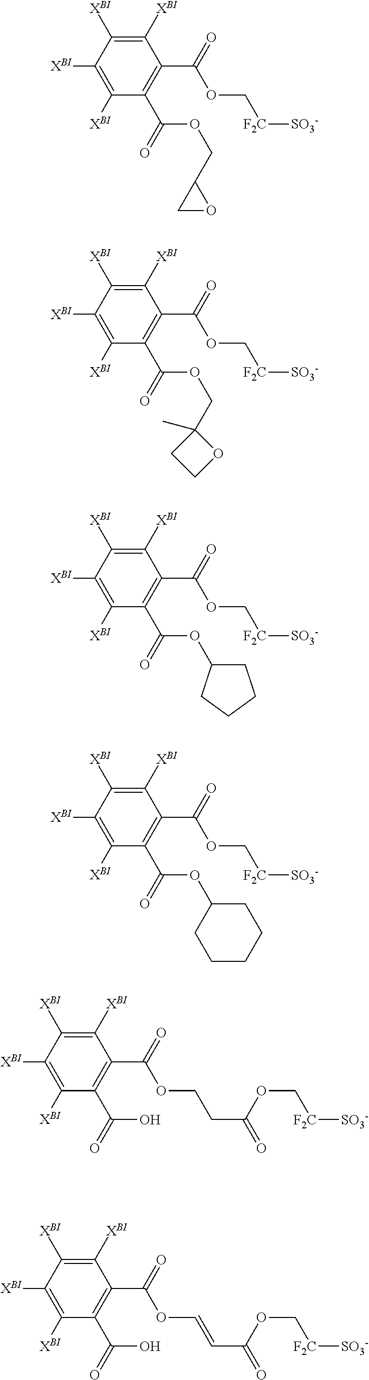

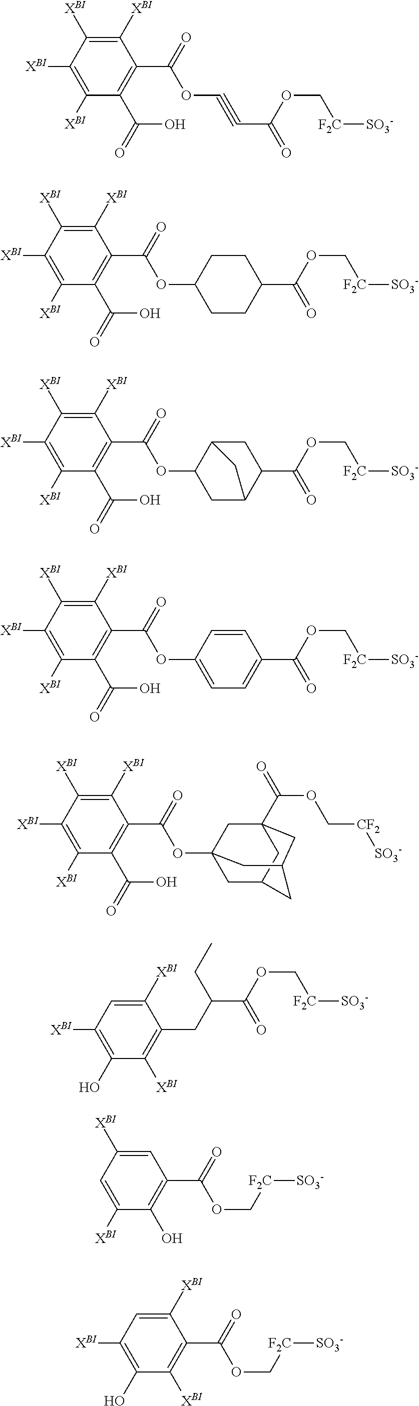

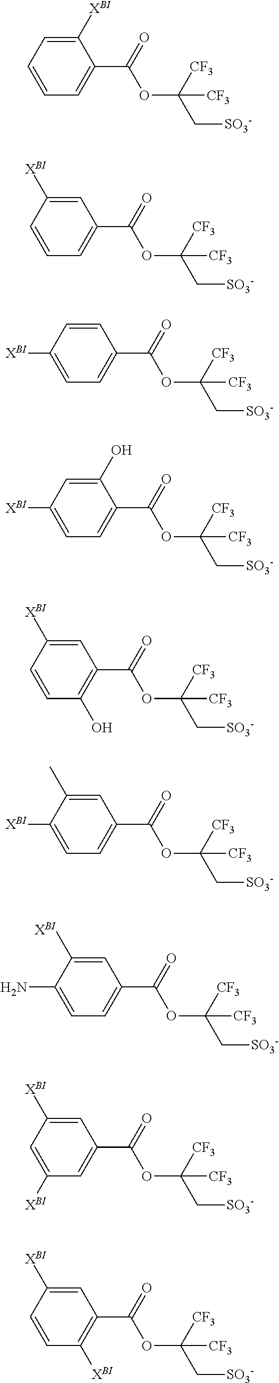

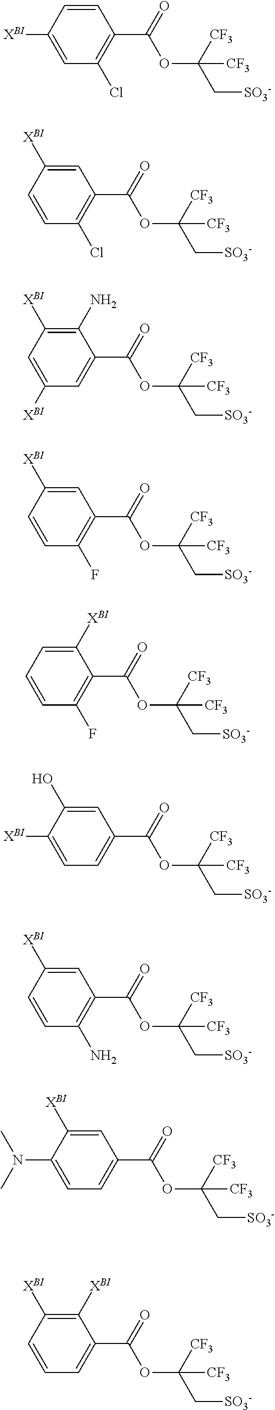

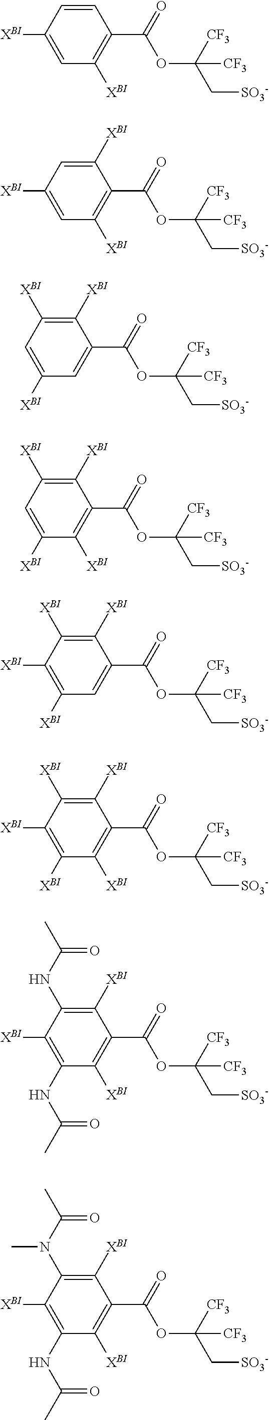

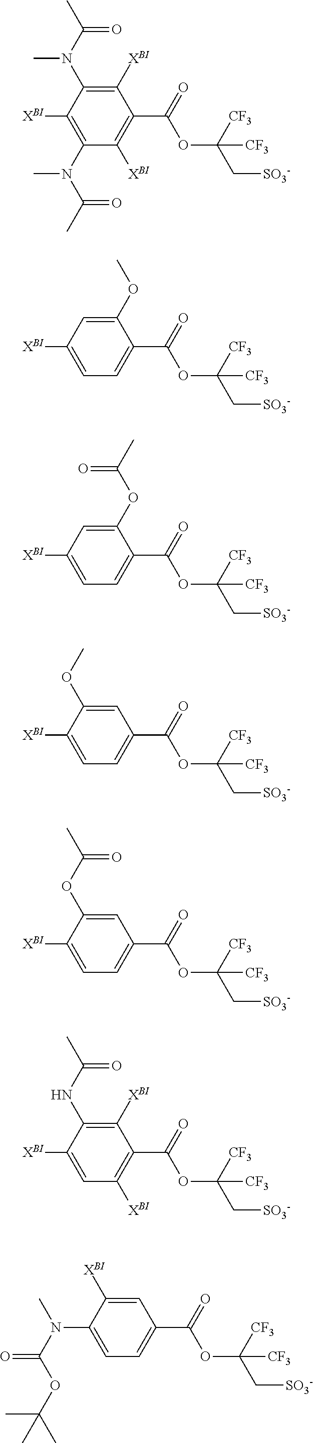

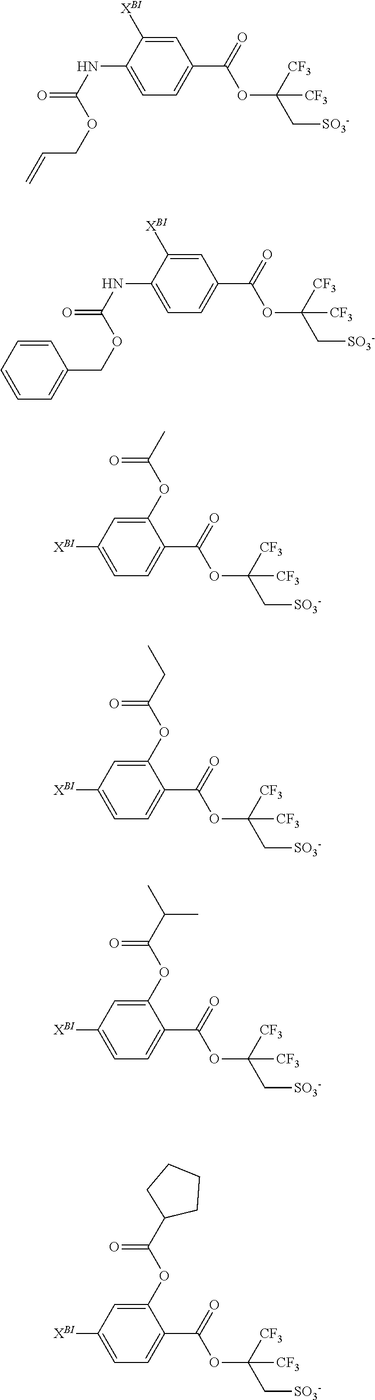

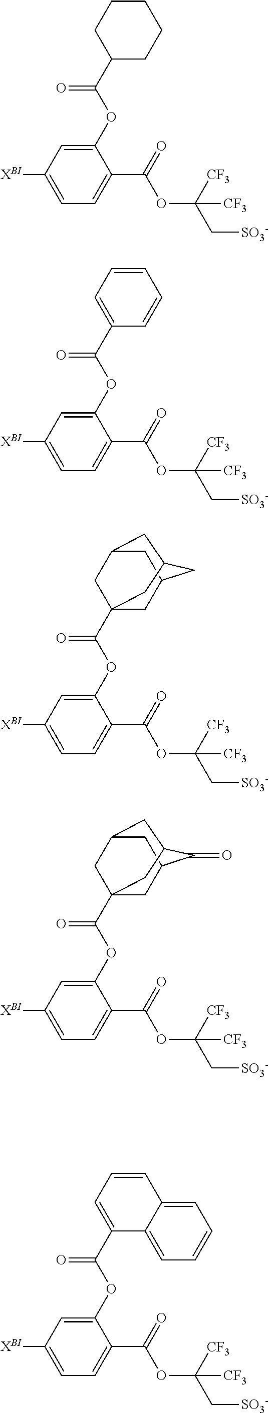

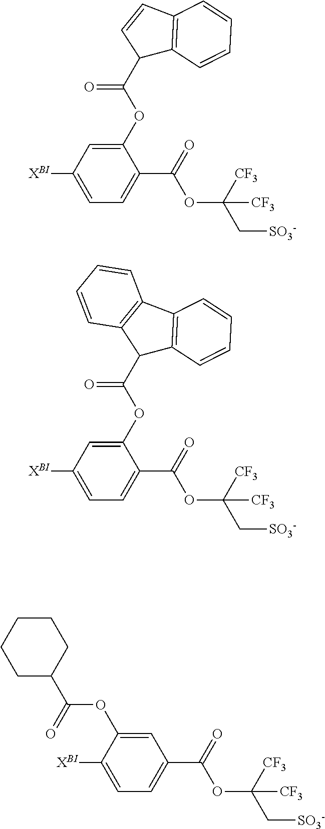

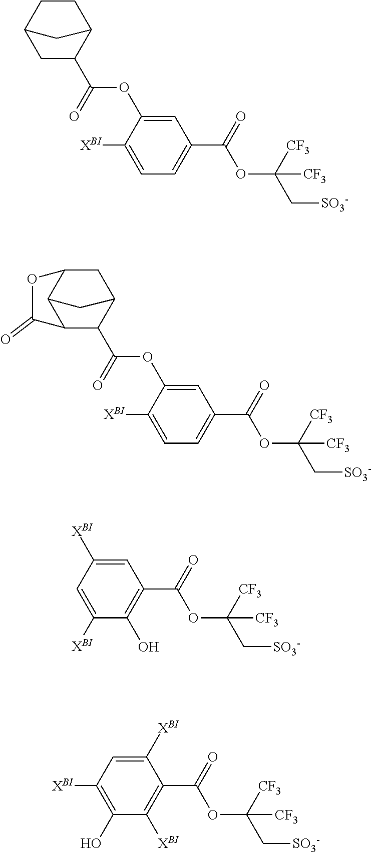

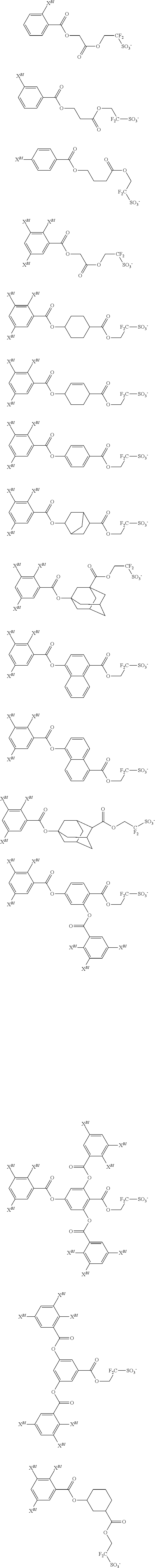

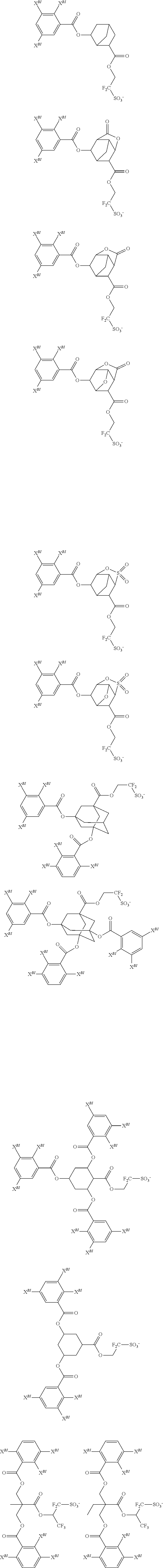

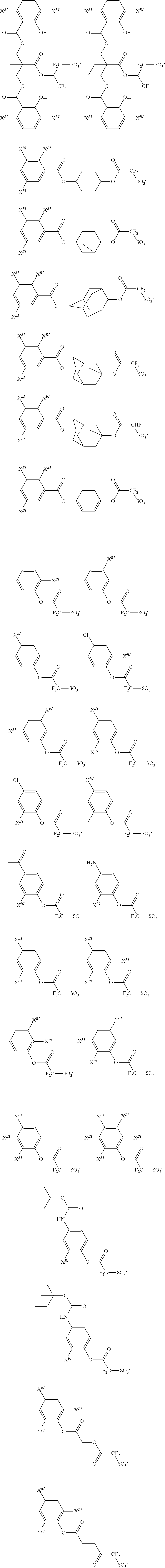

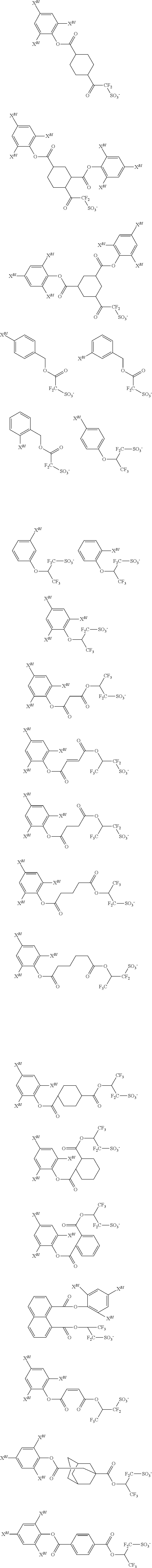

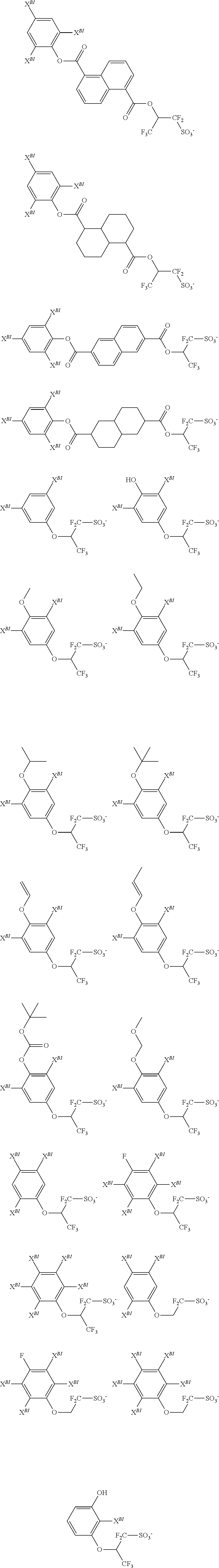

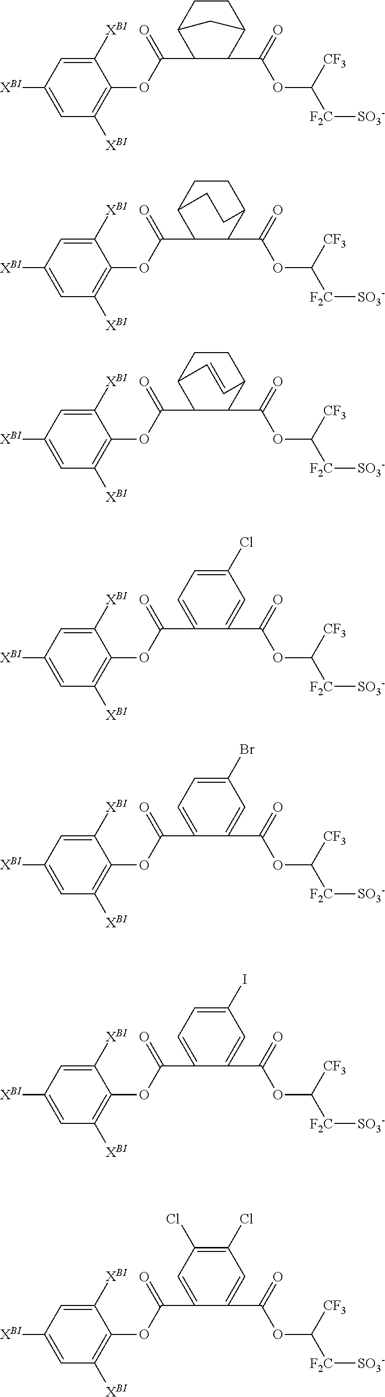

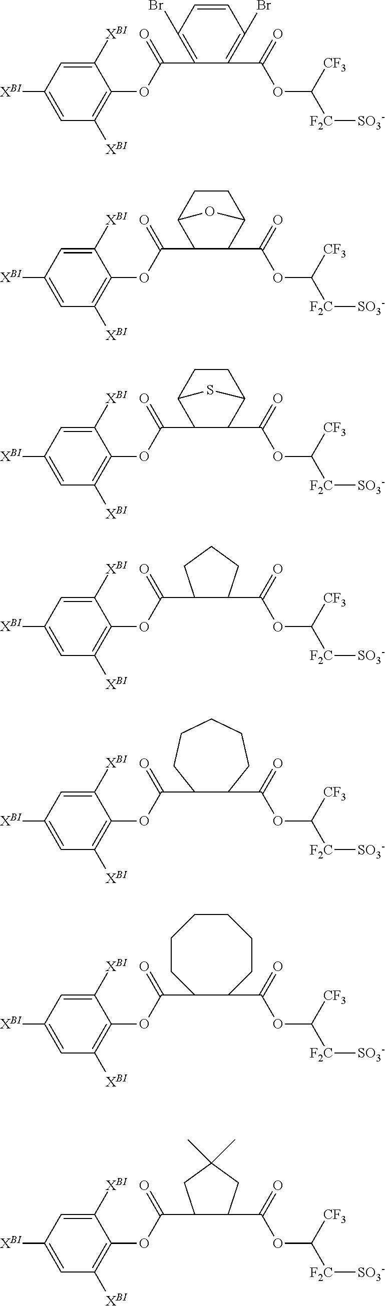

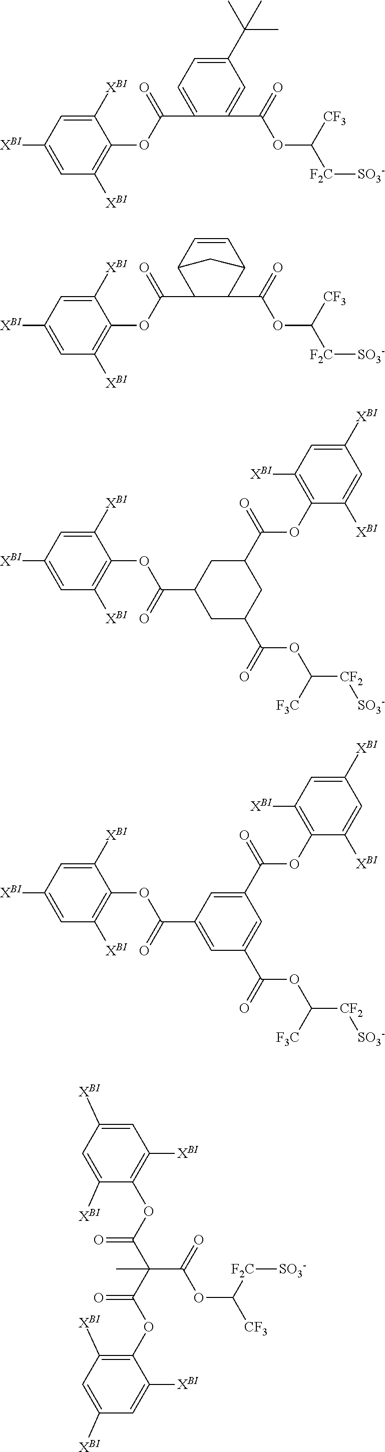

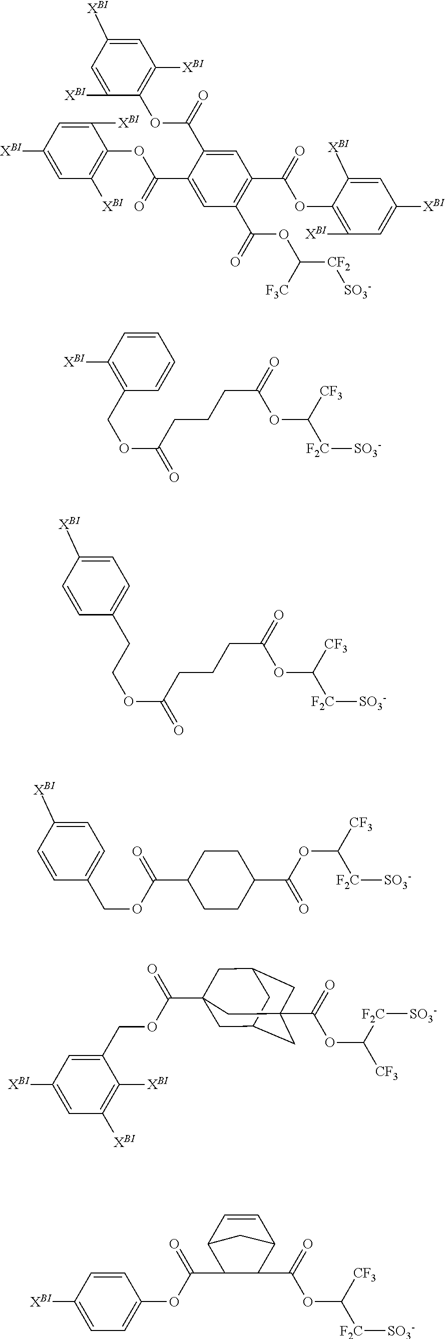

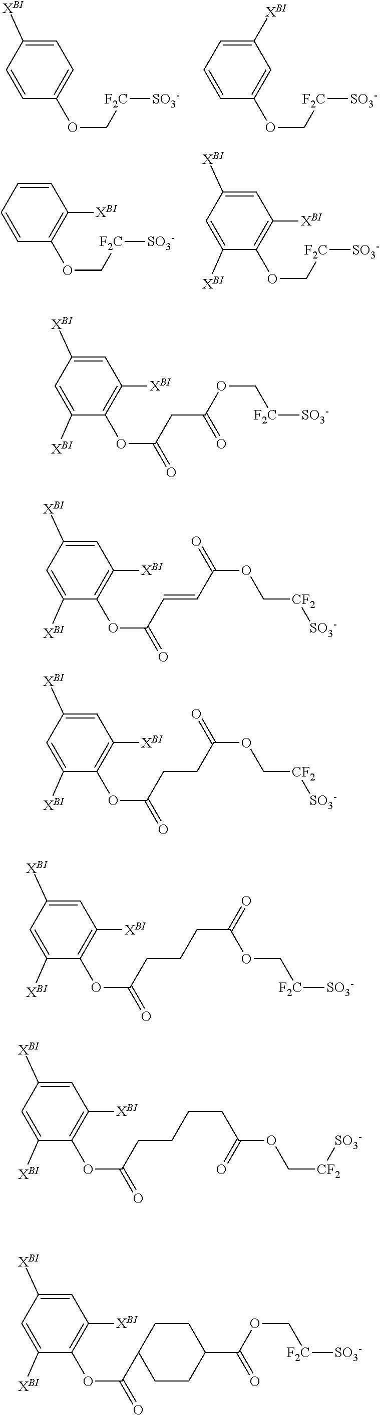

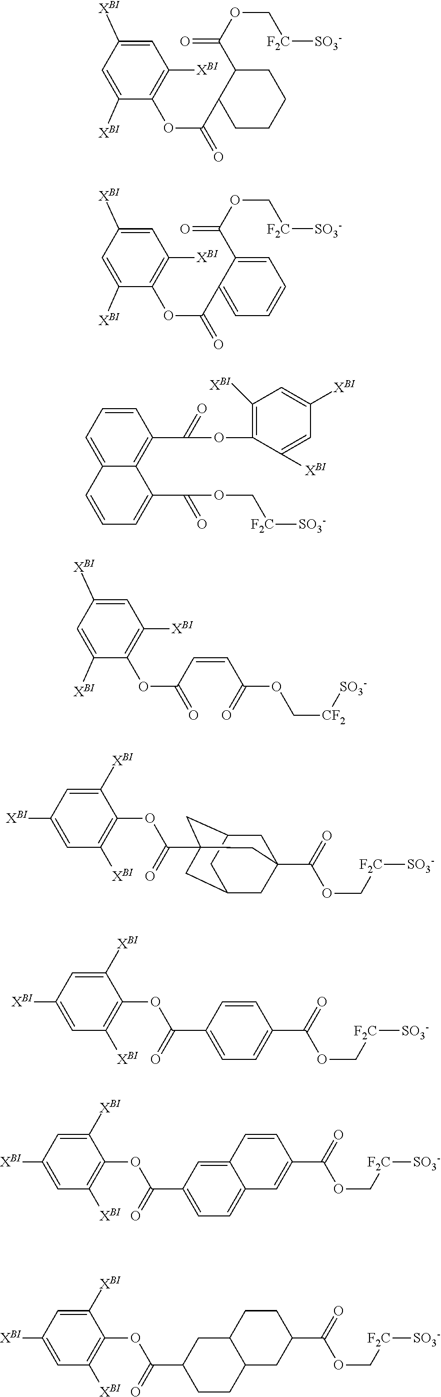

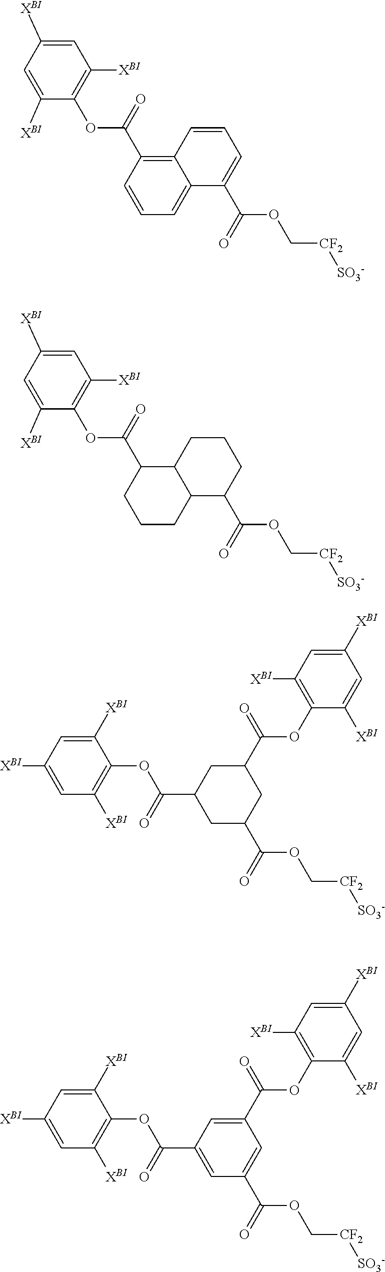

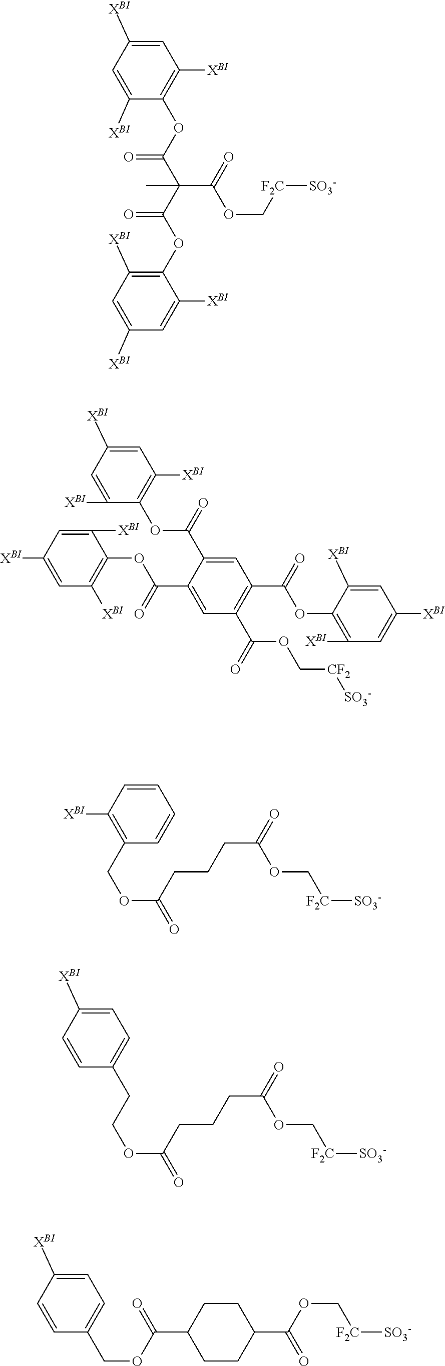

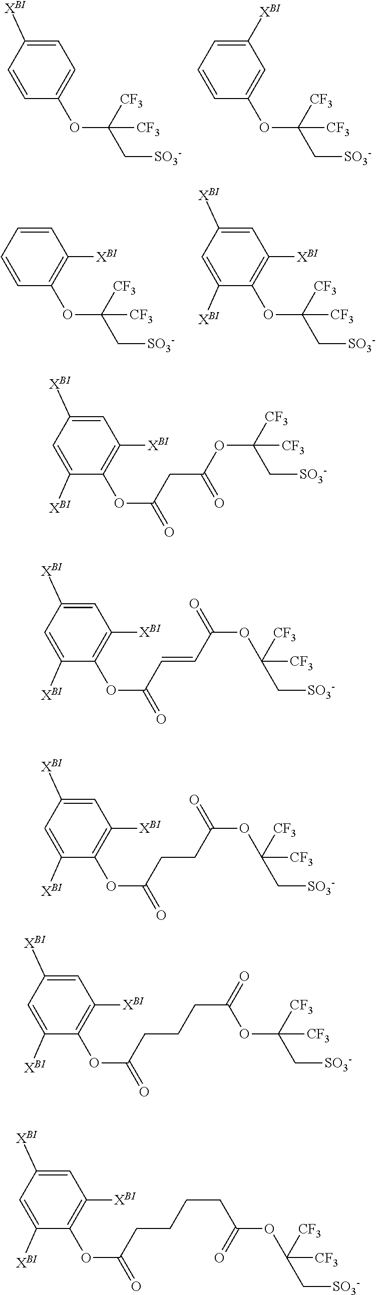

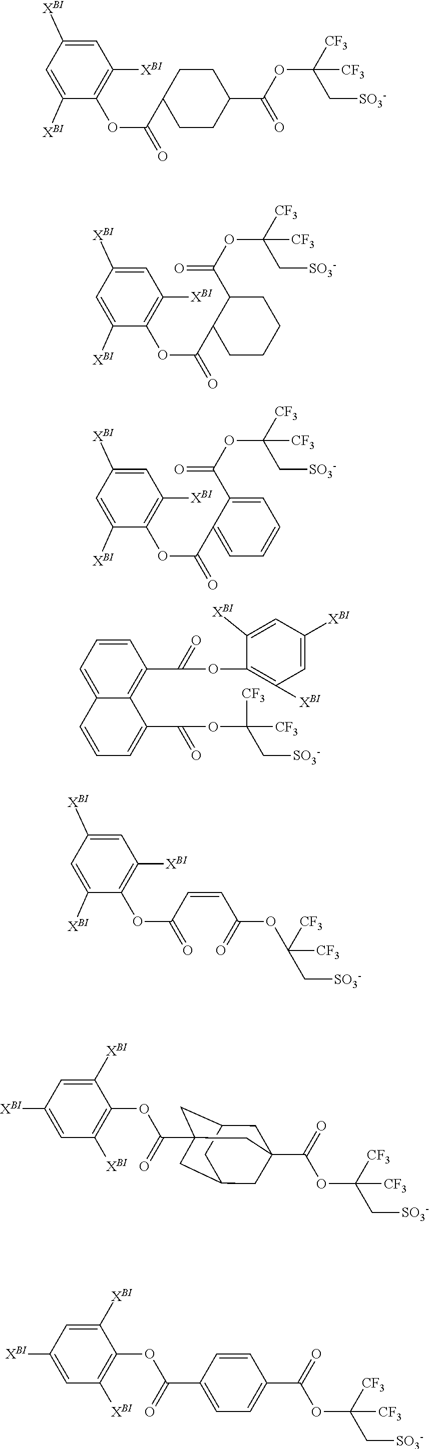

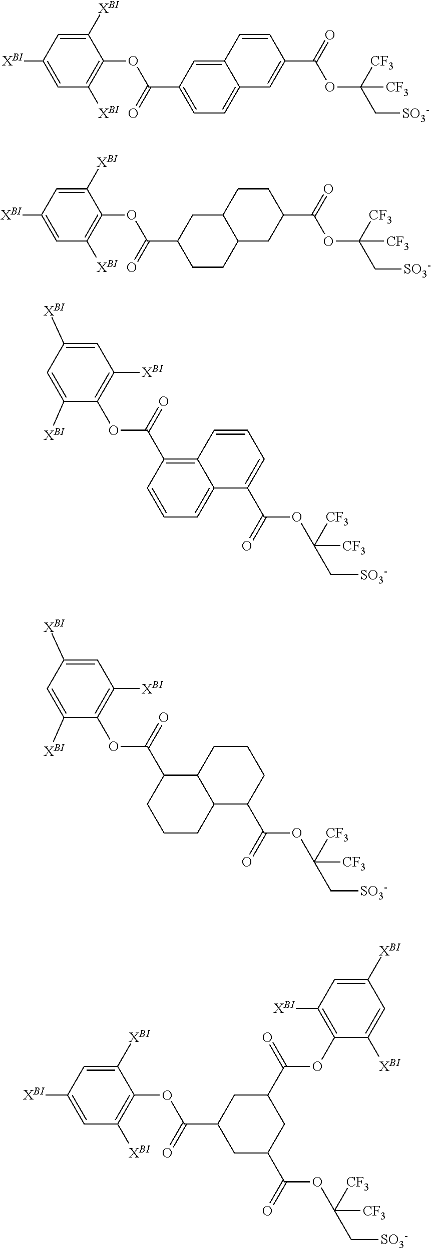

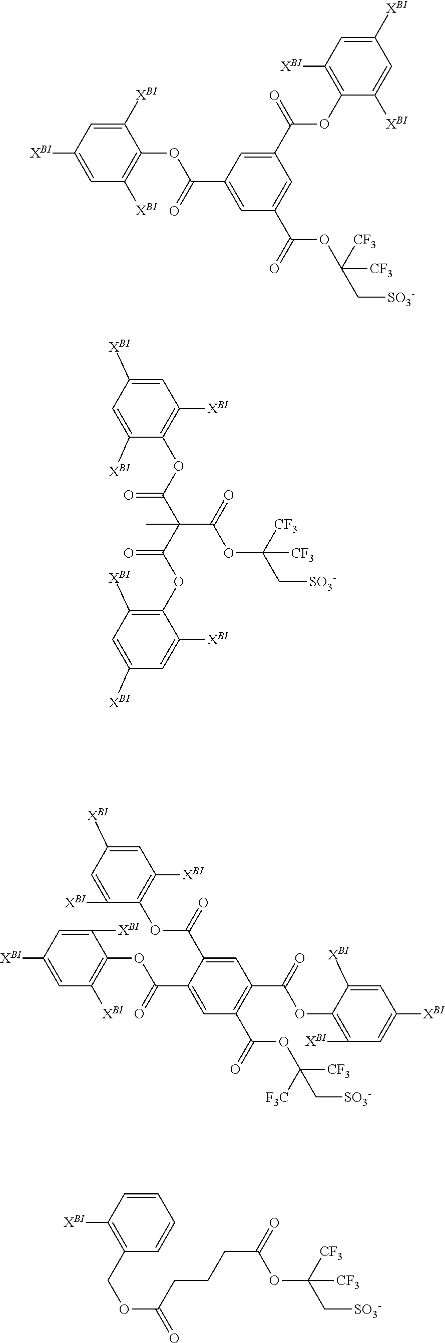

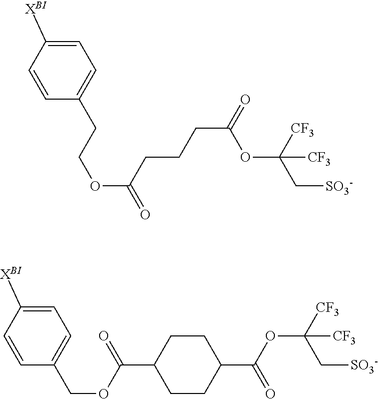

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-70692, anions having a cyclic ether group as described in JP-A 2018-180525 and JP-A 2021-35935, and anions as described in JP-A 2018-92159.

Further useful examples of the non-nucleophilic counter ion include bulky fluorine free benzenesulfonic acid anions as described in JP-A 2006-276759, JP-A 2015 117200, JP-A 2016-65016, and JP-A 2019-202974; fluorine-free benzenesulfonic acid or alkylsulfonic acid anions having an iodized aromatic group bonded thereto as described in JP 6645464.

Also useful are bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-24989.

In formulae (c2) and (c3), d1 and d2 are each independently 0, 1, 2 or 3, preferably 1.

In formula (c4), e1 is 0 or 1. The subscript e2 is 0, 1, 2, 3 or 4. The subscript e3 is 0, 1, 2, 3 or 4. It is noted that e2+e3 is from 0 to 4 when e1=0, e2+e3 is from 0 to 6 when e1=1.

In formulae (c2), (c3) and (c4), L¹ is a single bond, ether bond, ester bond, carbonyl group, sulfonic ester bond, sulfonamide bond, carbonate bond, or carbamate bond. From the aspect of synthesis, an ether bond, ester bond or carbonyl group is preferred, with the ester bond or carbonyl being more preferred.

In formula (c2), Rf¹ and Rf² are each independently fluorine or a C₁-C₆ fluorinated saturated hydrocarbyl group. It is preferred that both Rf¹ and Rf² be fluorine because the generated acid has a higher acid strength. Rf³ and Rf⁴ are each independently hydrogen, fluorine, or a C₁-C₆ fluorinated saturated hydrocarbyl group. It is preferred for solvent solubility that at least one of Rf³ and Rf⁴ be trifluoromethyl.

In formula (c3), Rf⁵ and Rf⁶ are each independently hydrogen, fluorine or a C₁-C₆ fluorinated saturated hydrocarbyl group. It is excluded that all Rf⁵ and Rf⁶ are hydrogen at the same time. It is preferred for solvent solubility that at least one of Rf⁵ and Rf⁶ be trifluoromethyl.

In formula (c4), Rf⁷ is a fluorine, C₁-C₆ fluorinated alkyl group, C₁-C₆ fluorinated alkoxy group or C₁-C₆ fluorinated alkylthio moiety. Rf⁷ is preferably a fluorine, trifluoromethyl, difluoromethyl, trifluoromethoxy, difluoromethoxy, trifluoromethylthio or difluoromethylthio moiety, more preferably a fluorine, trifluoromethyl or trifluoromethoxy moiety. A plurality of Rf⁷ may be identical or different when f is 2, 3 or 4.

In formula (c4), R³³ is halogen exclusive of iodine, or a C₁-C₂₀ hydrocarbyl group which may contain a heteroatom. The hydrocarbyl group may be saturated or unsaturated, and may be straight, branched, or cyclic. Examples thereof are as exemplified for the hydrocarbyl groups R¹ to R⁴ in formula (1), but not limited thereto. A plurality of R³³ may be identical or different when e3 is 2, 3 or 4.

A plurality of R³³ may bond together to form a ring with the carbon atoms to which they are attached when e3 is 2, 3 or 4. Specific examples of the ring formed herein include cyclopropane, cyclobutane, cyclopentane, cyclohexane, norbornane and adamantane rings. In the ring, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the ring may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

Examples of the anion in repeat unit (c2) are shown below, but not limited thereto. In the following formulae, R^{A} is as defined above, and Me is a methyl group.

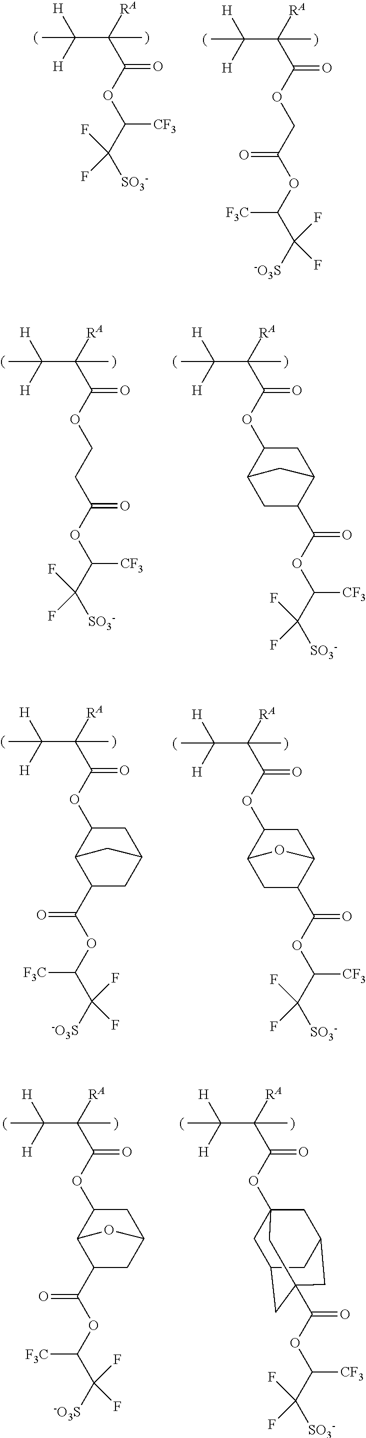

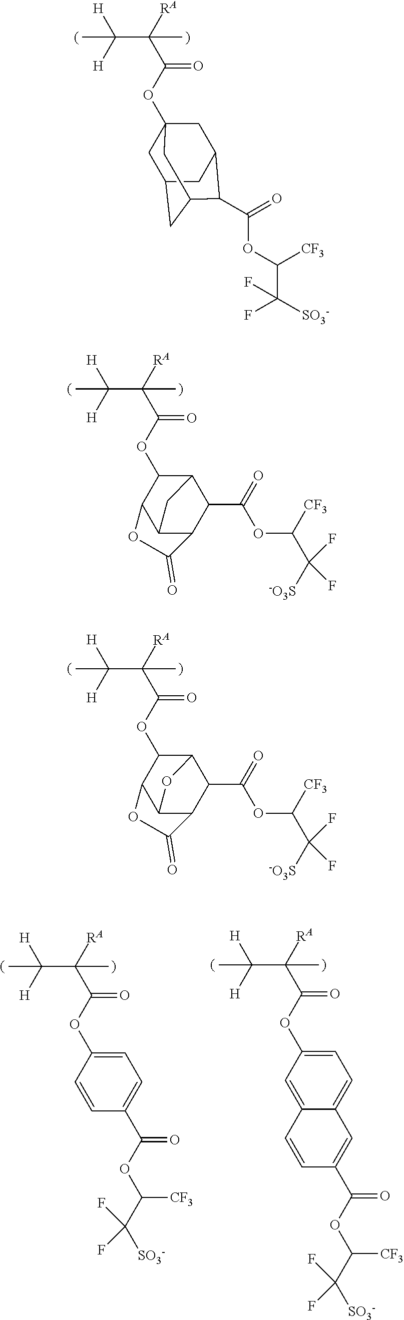

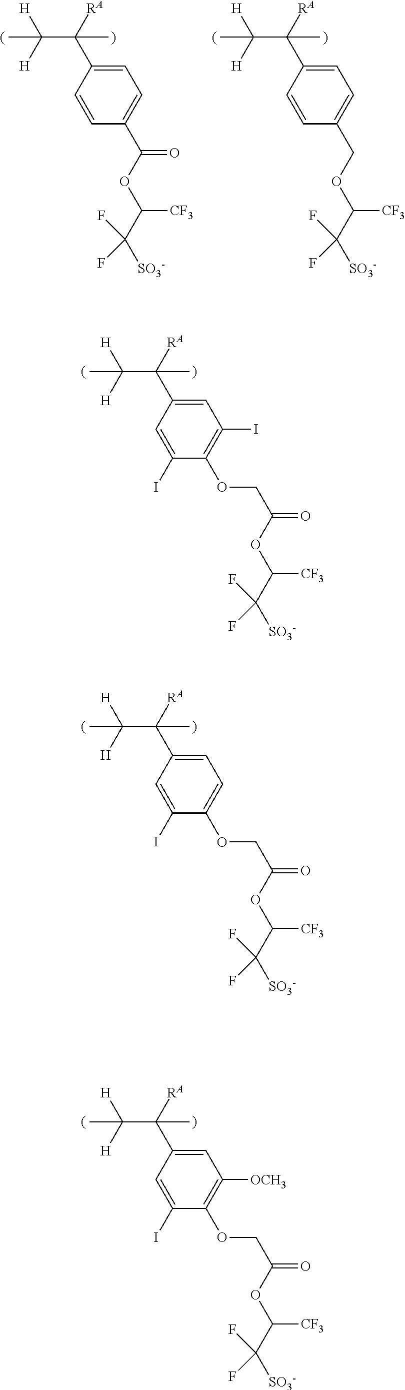

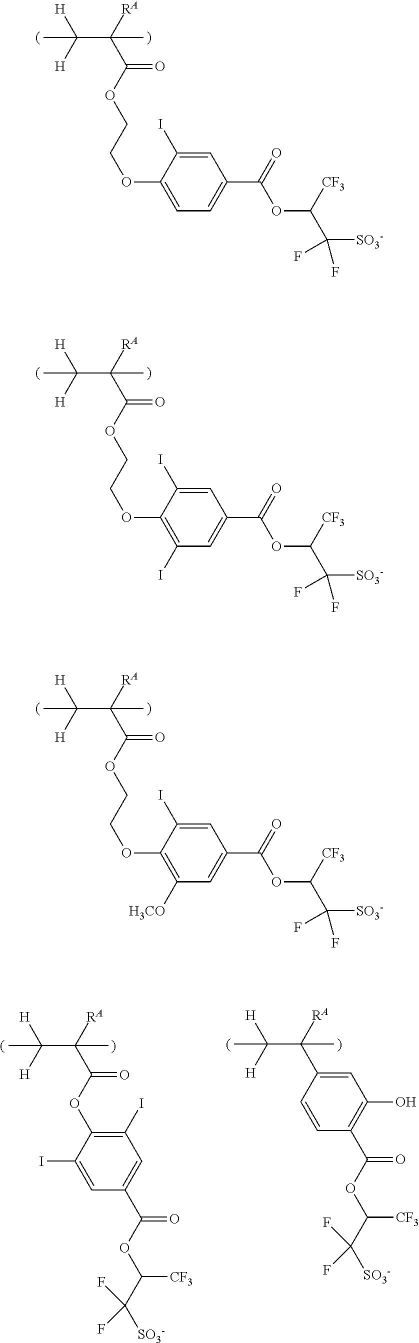

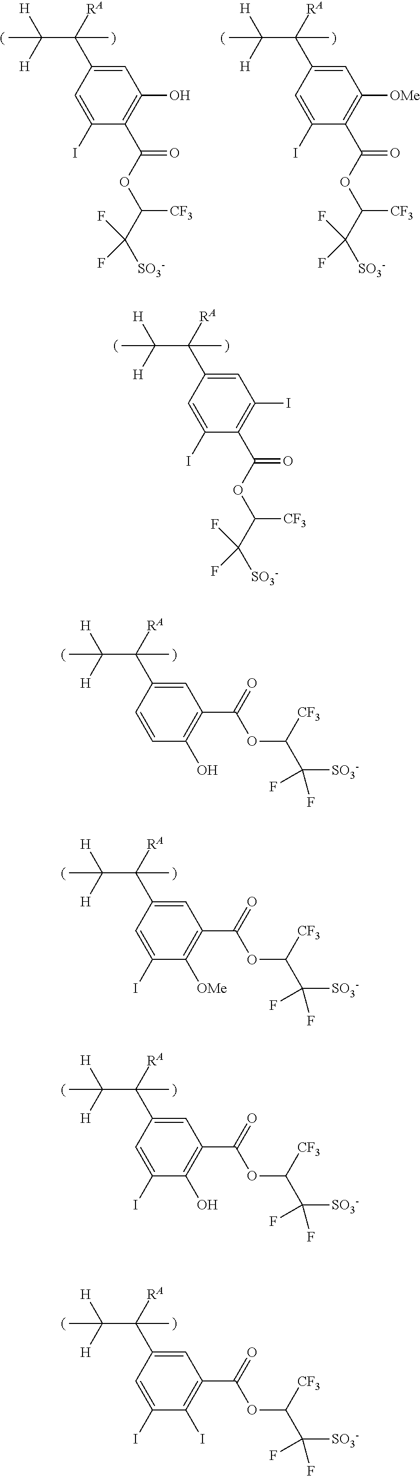

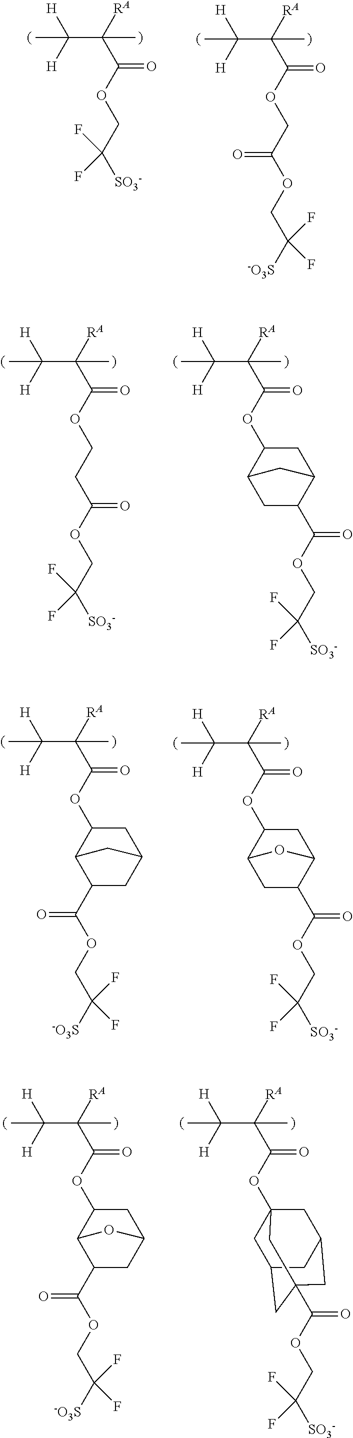

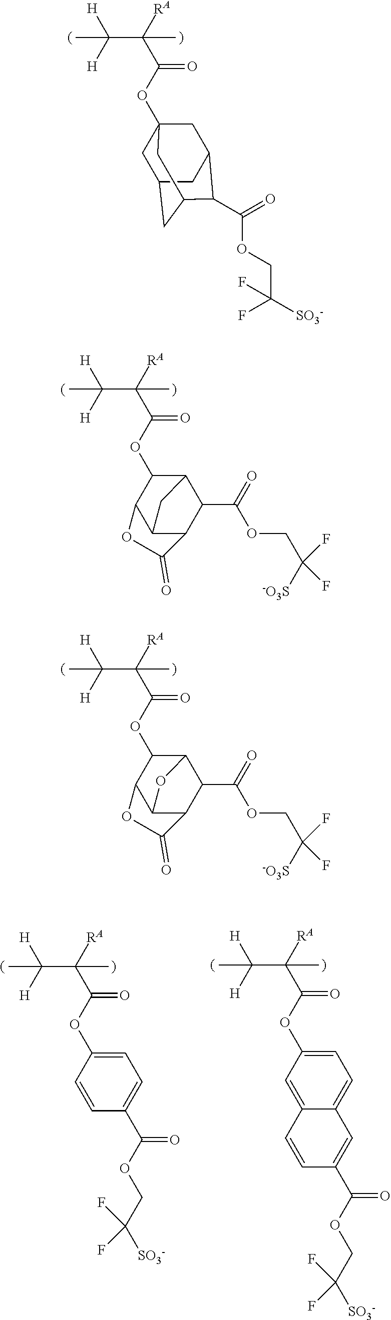

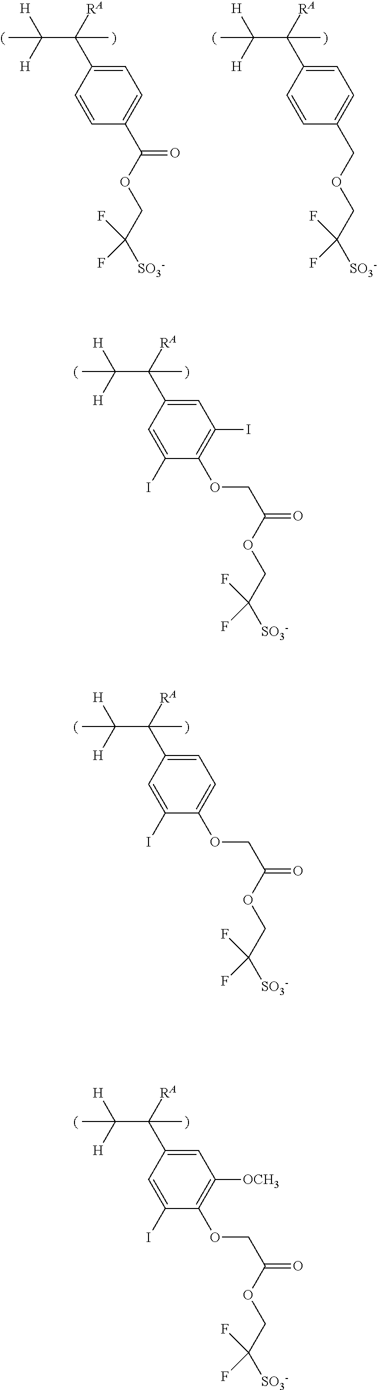

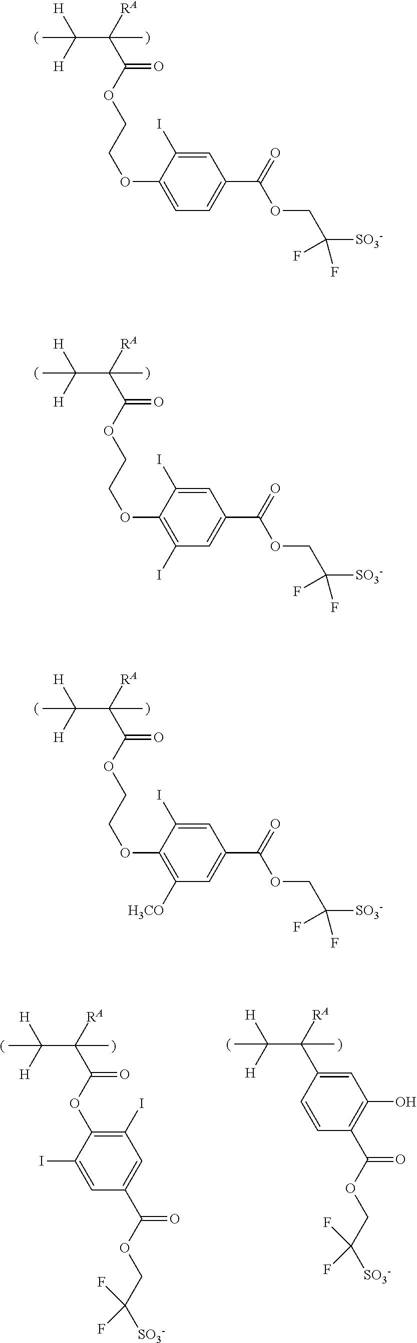

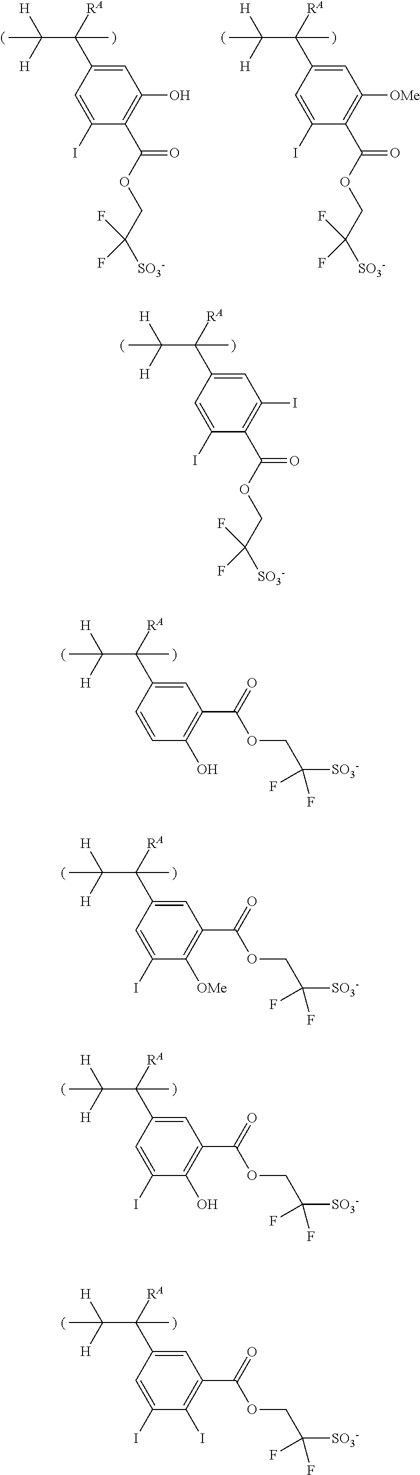

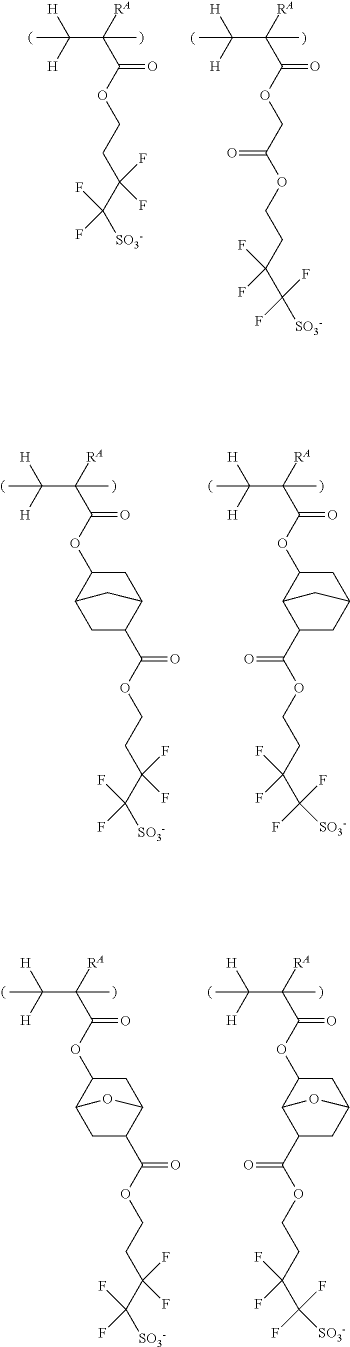

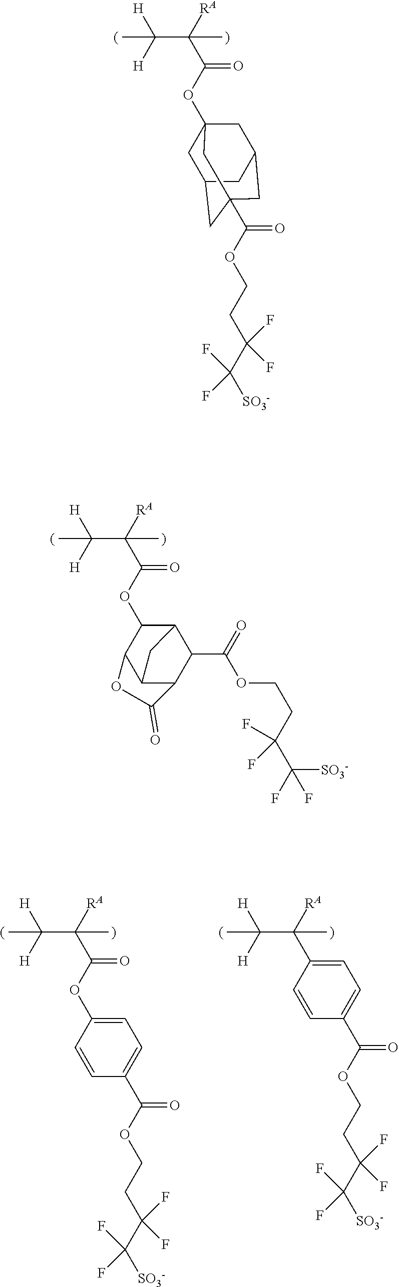

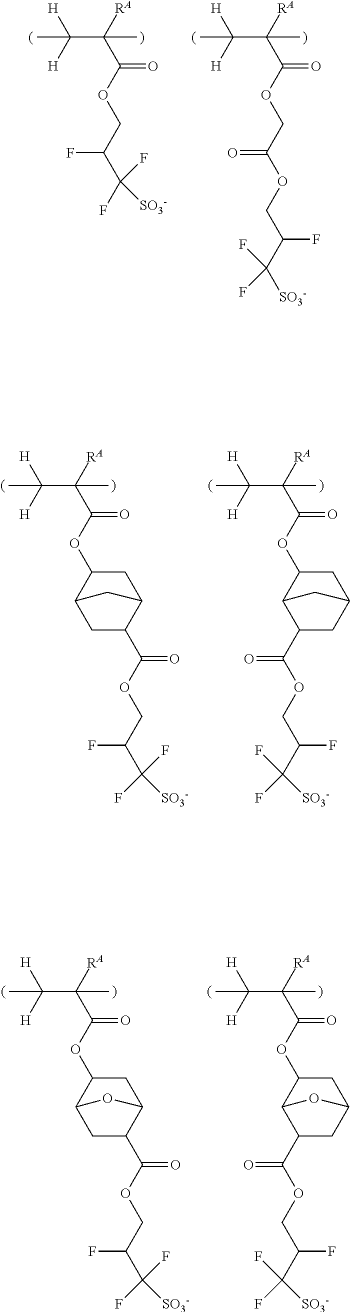

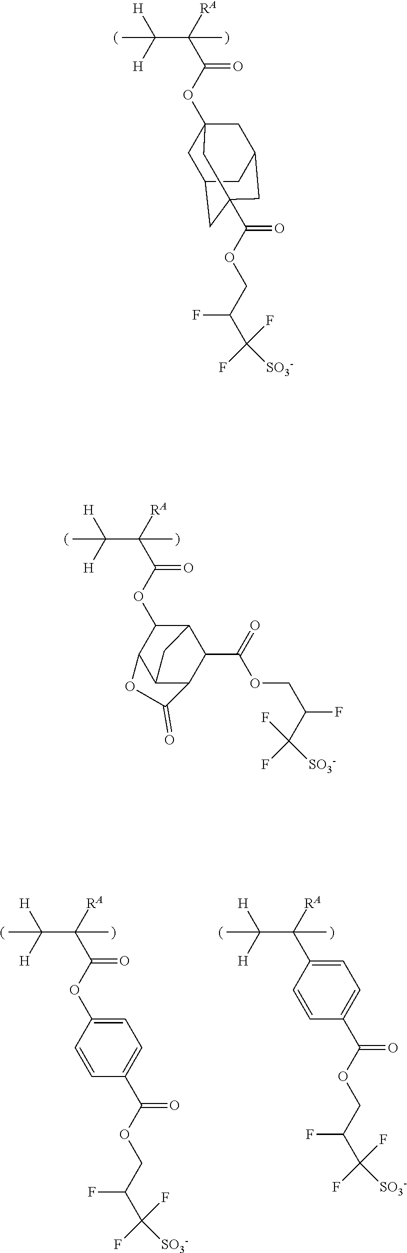

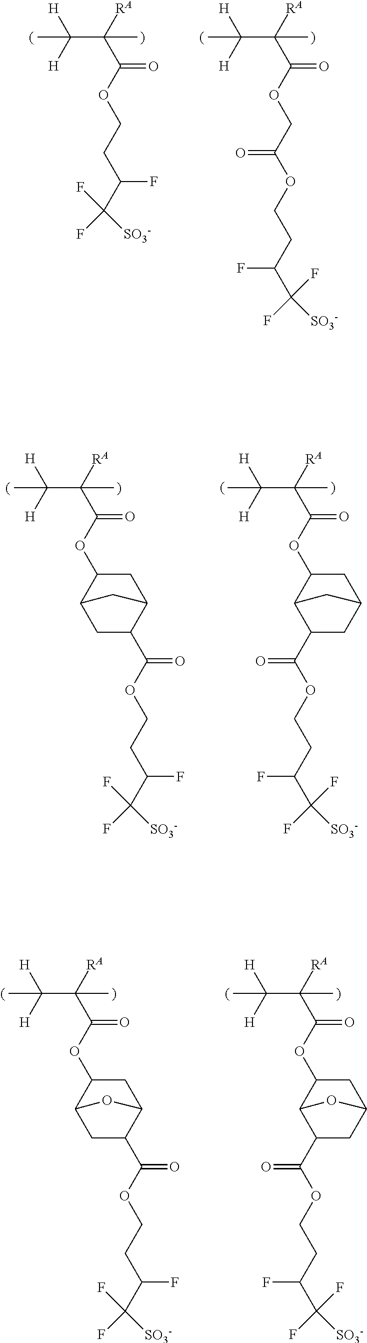

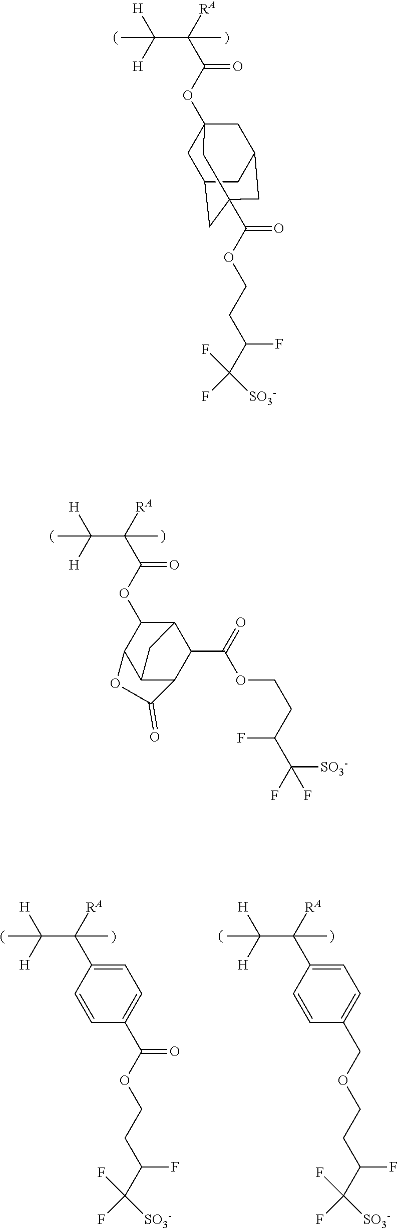

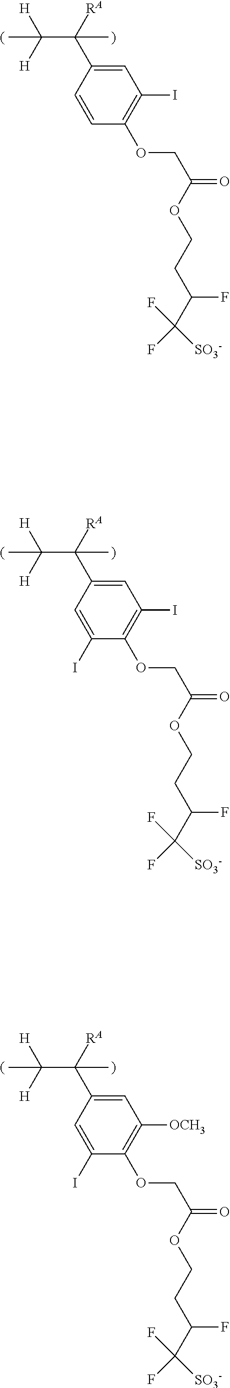

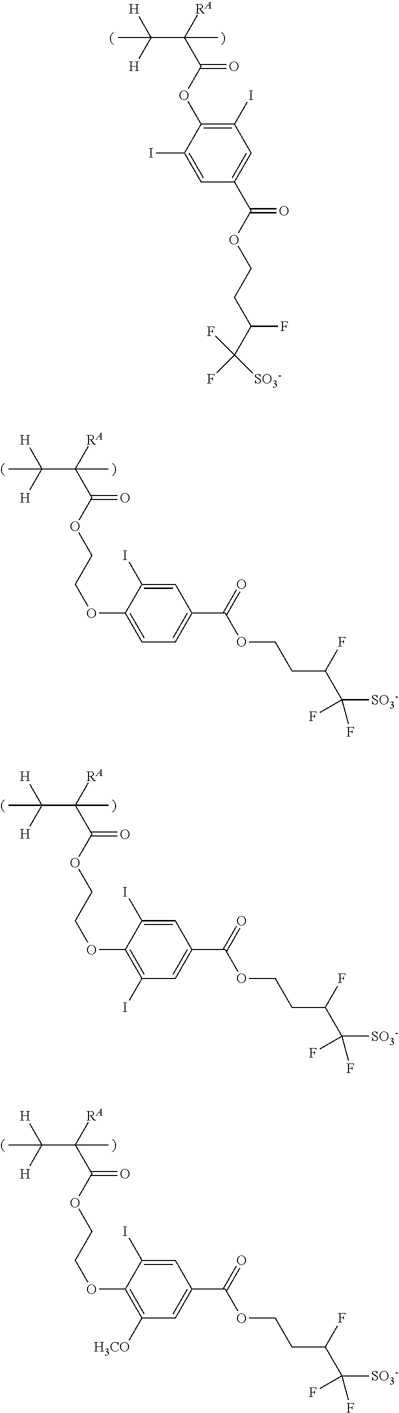

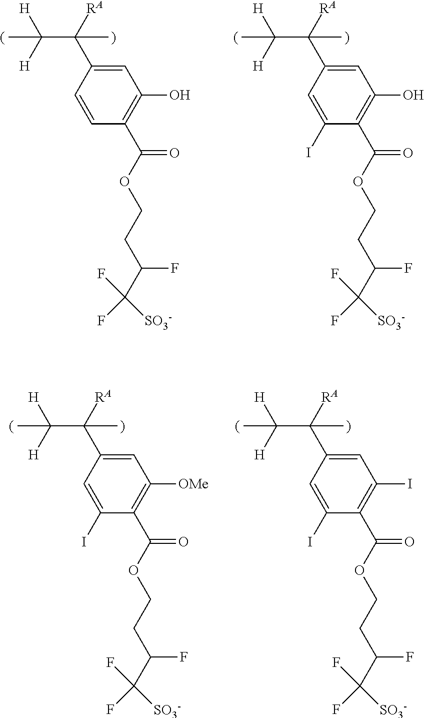

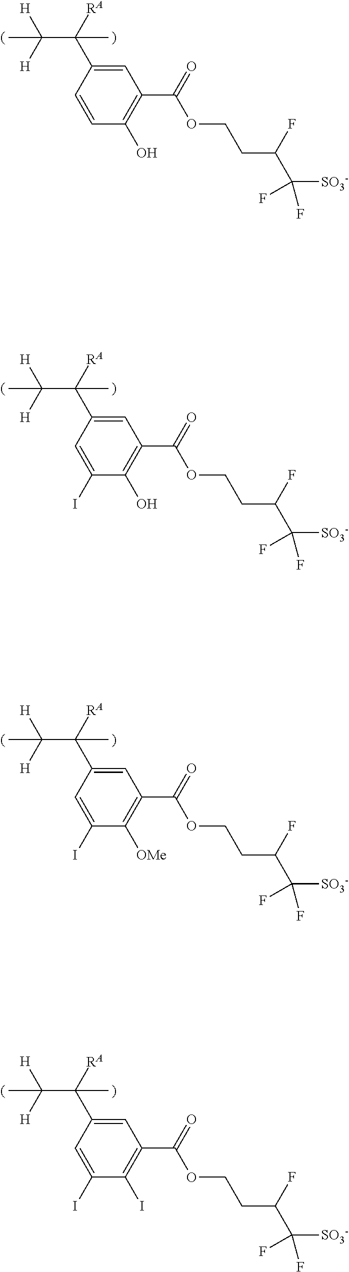

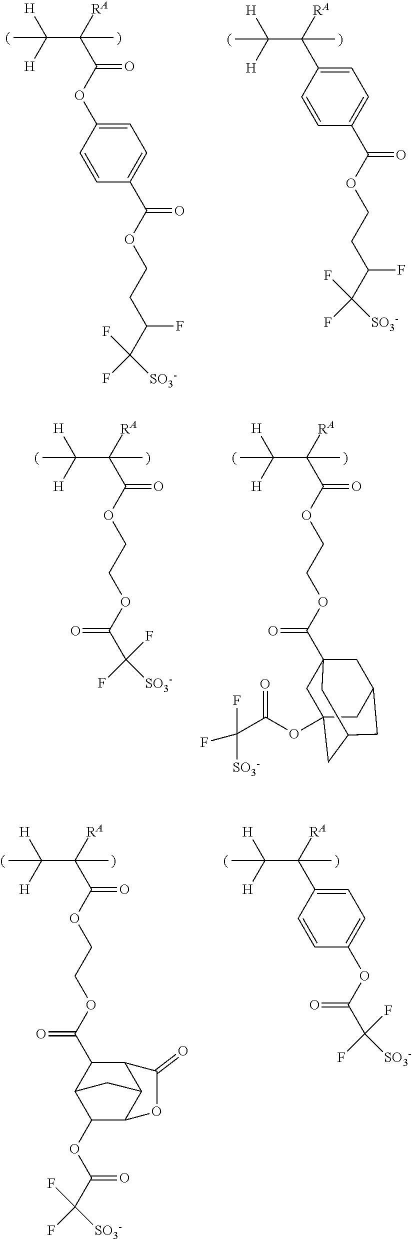

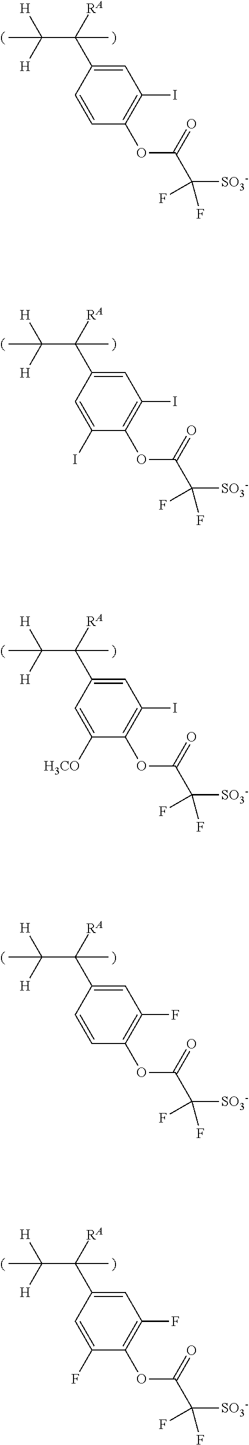

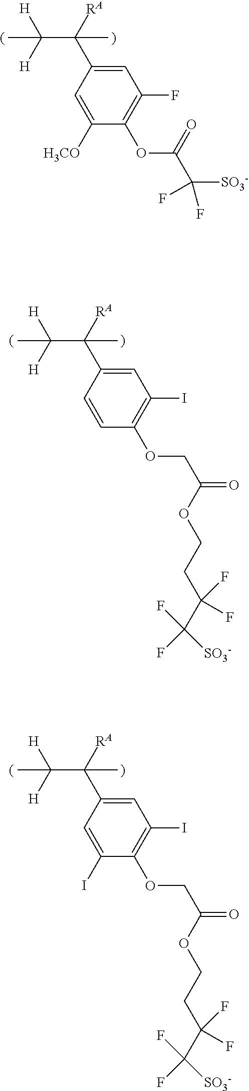

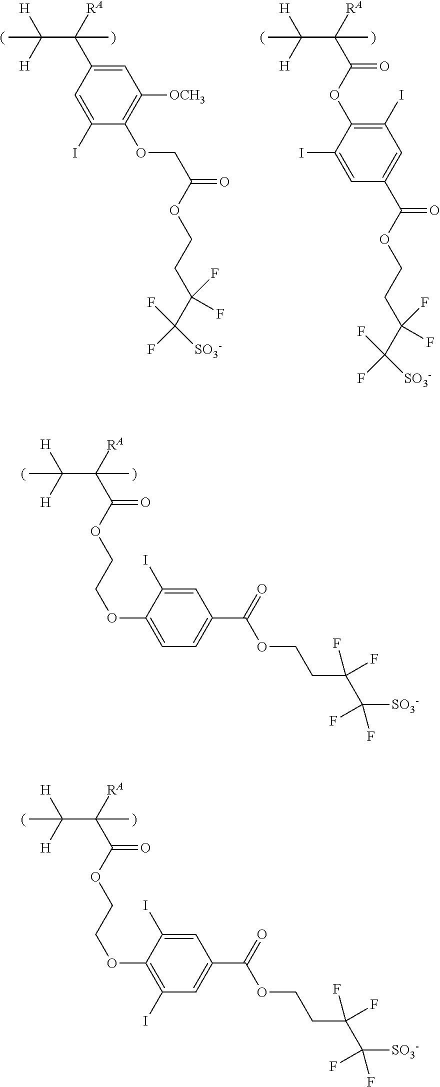

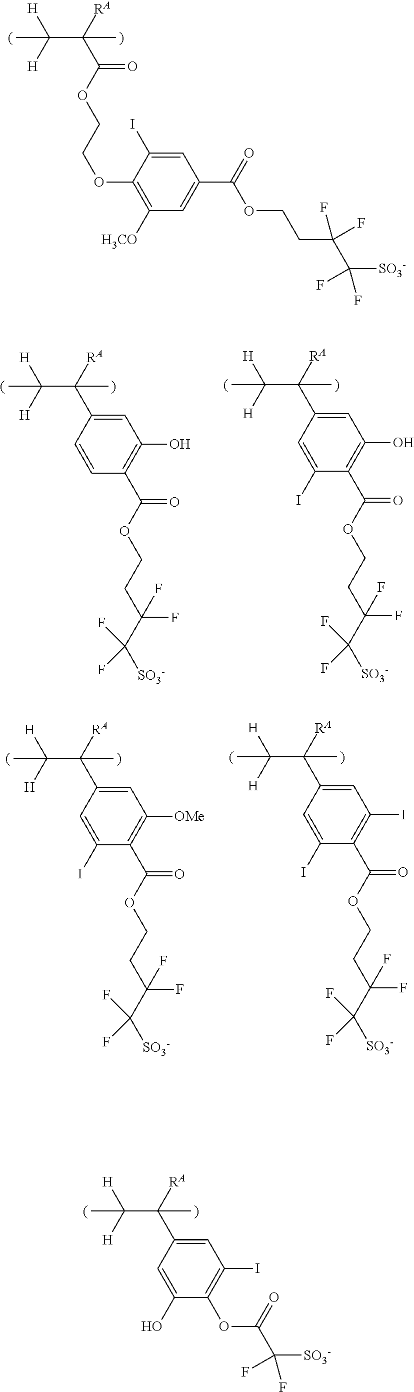

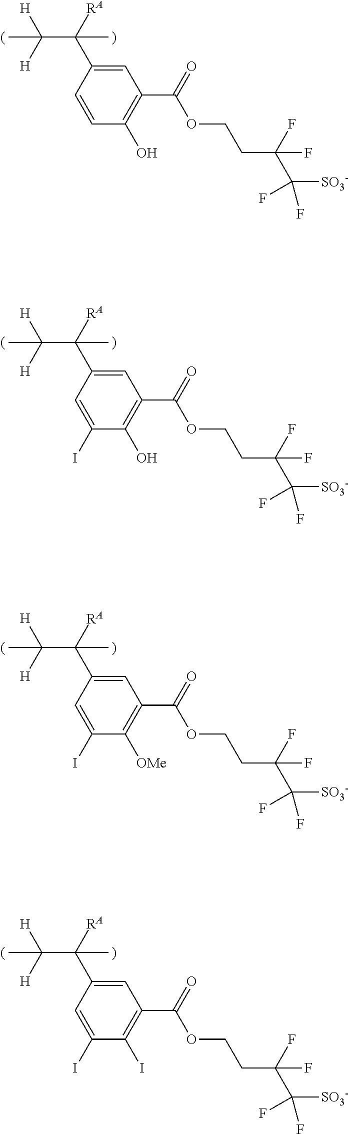

Examples of the anion in repeat unit c3 are shown below, but not limited thereto. Herein R¹ is as defined above.

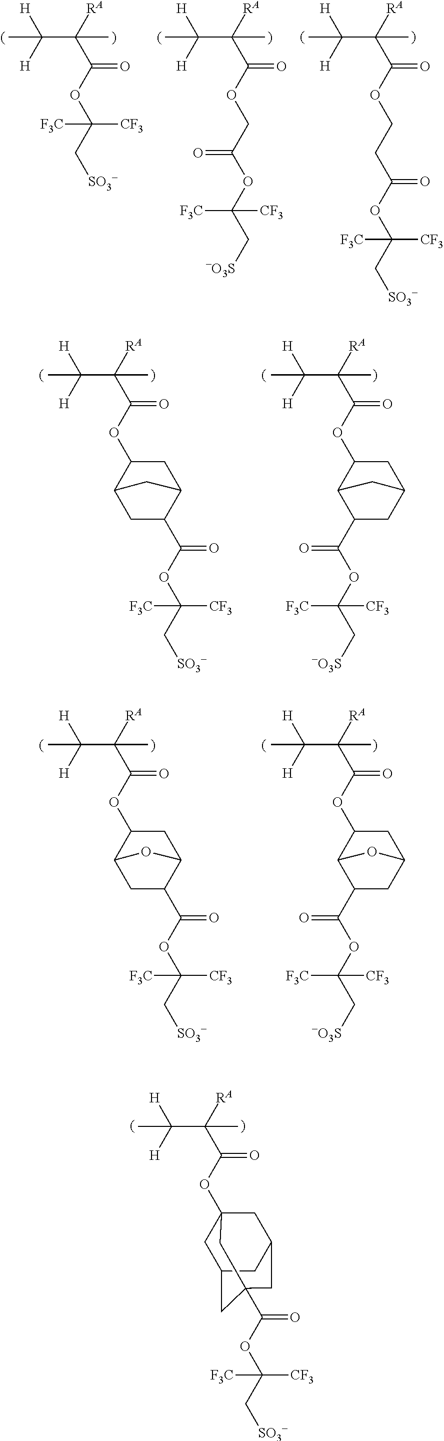

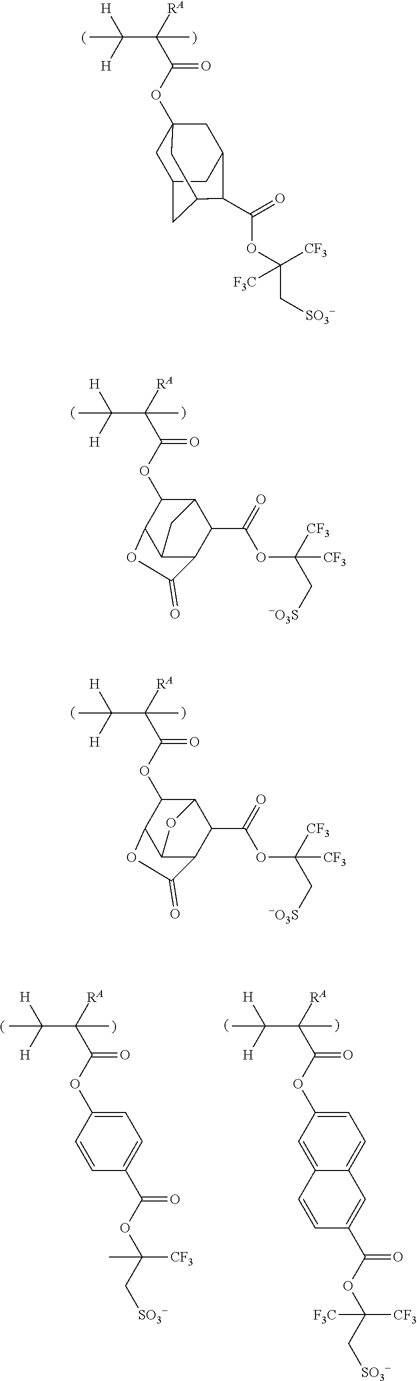

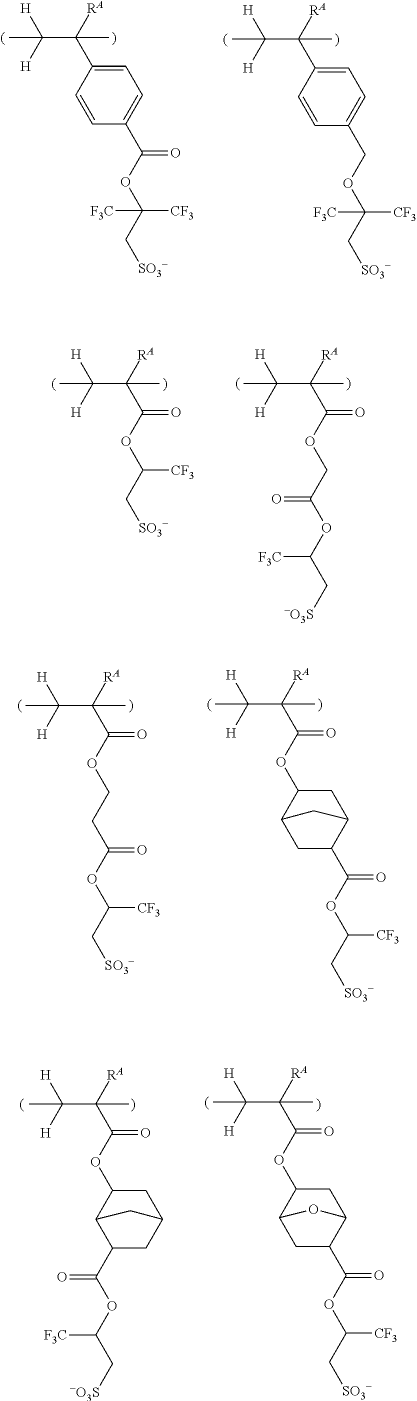

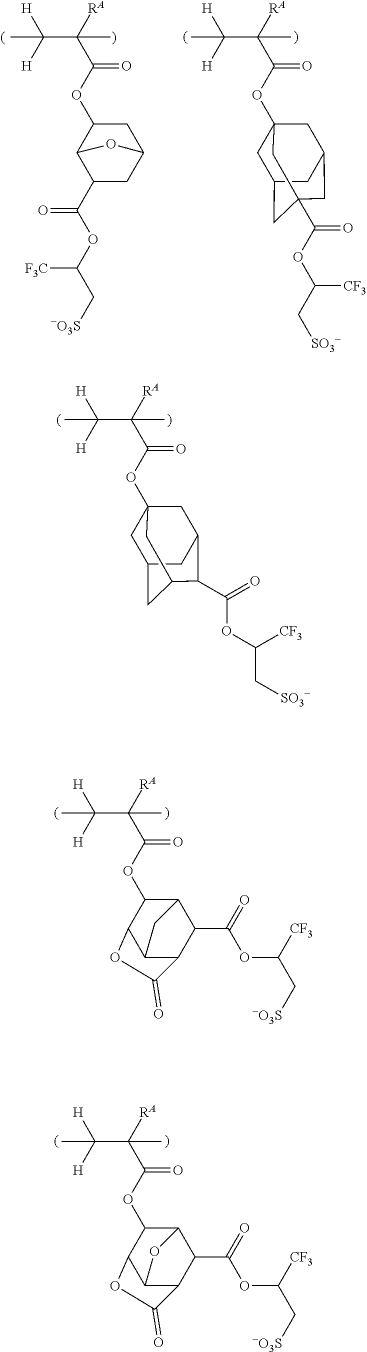

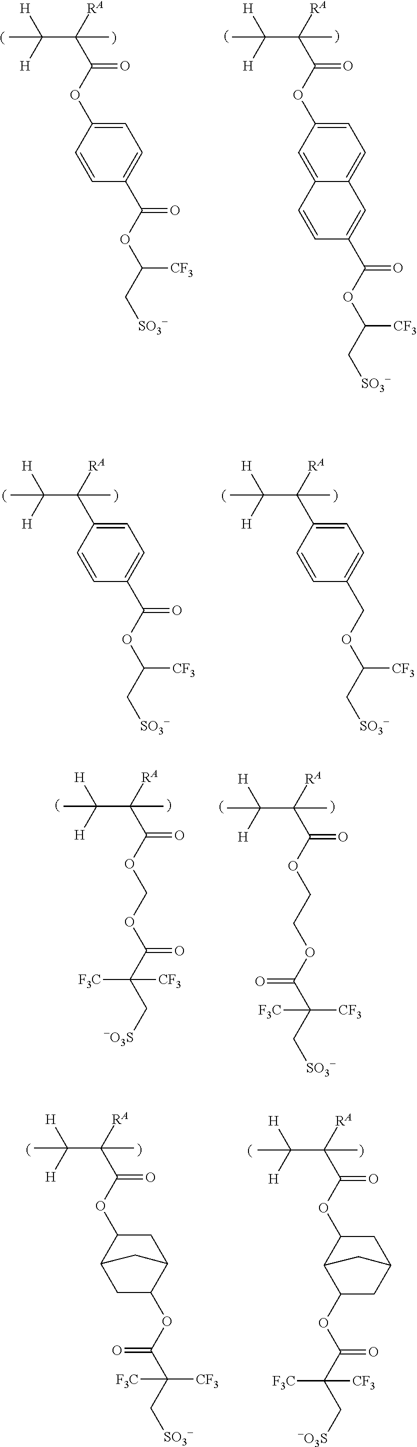

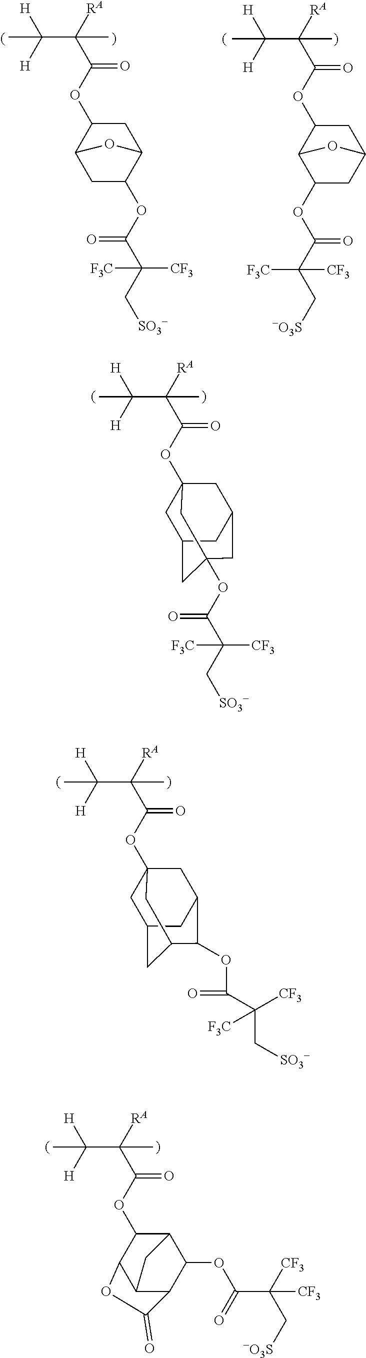

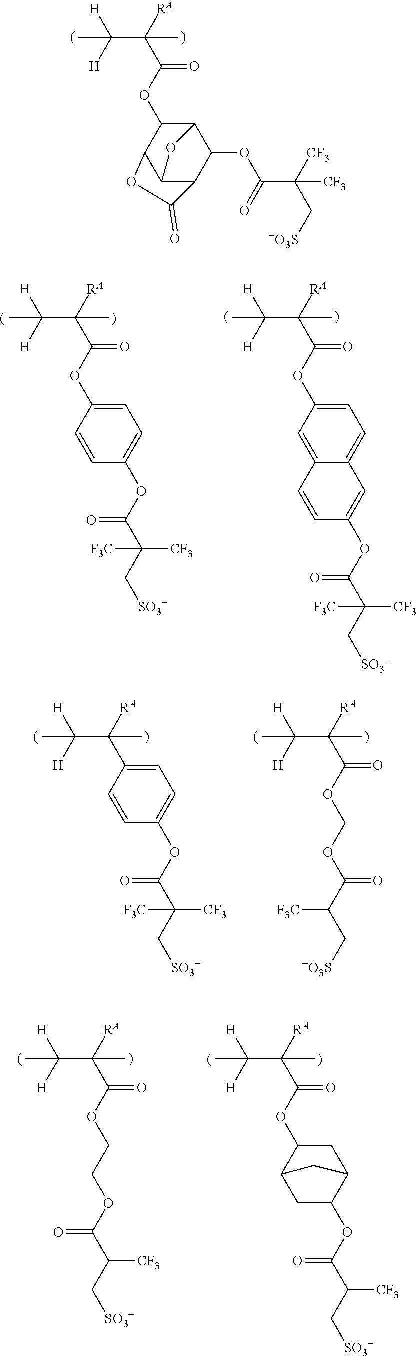

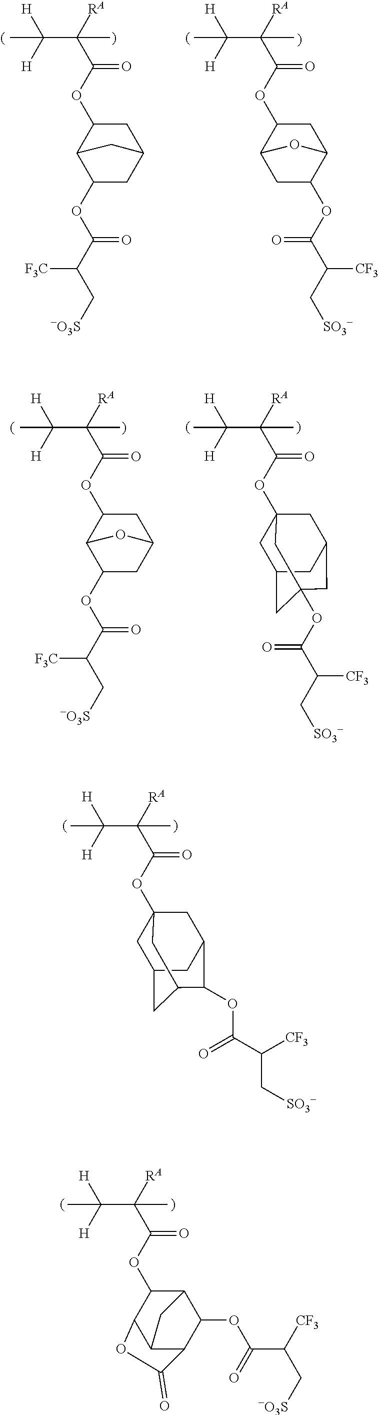

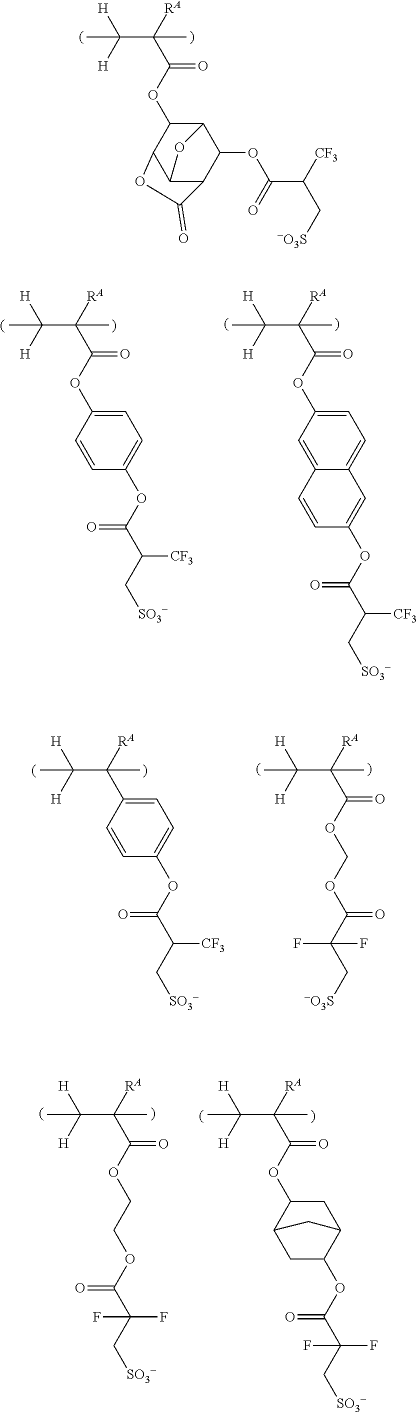

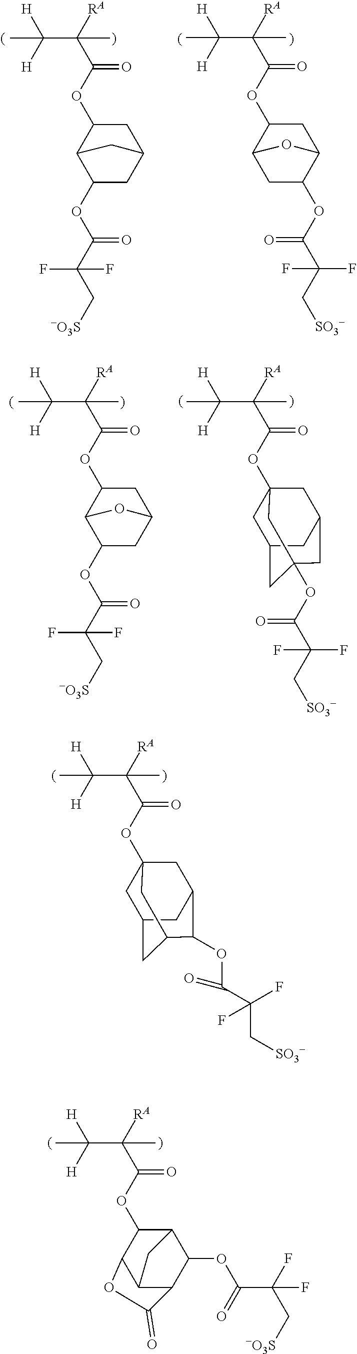

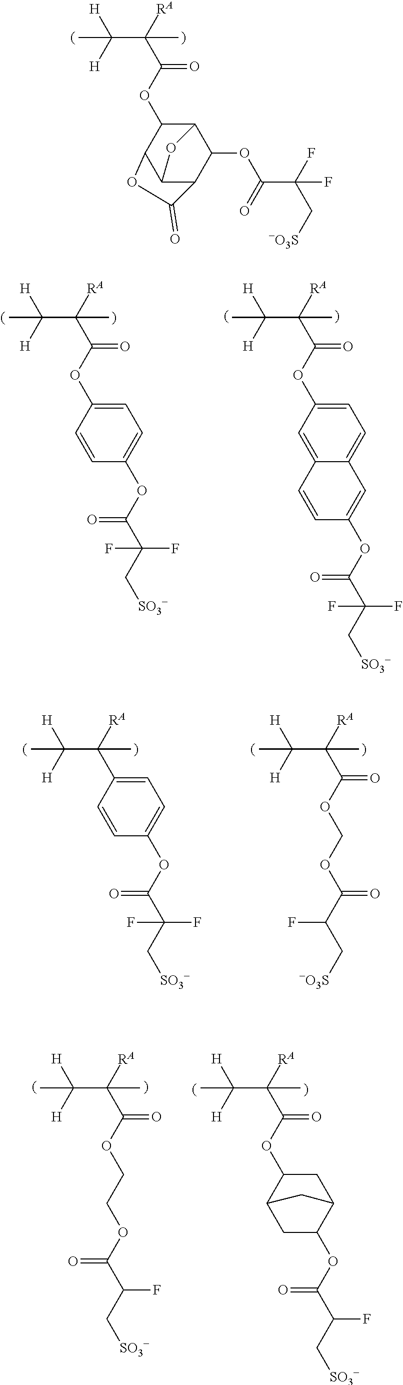

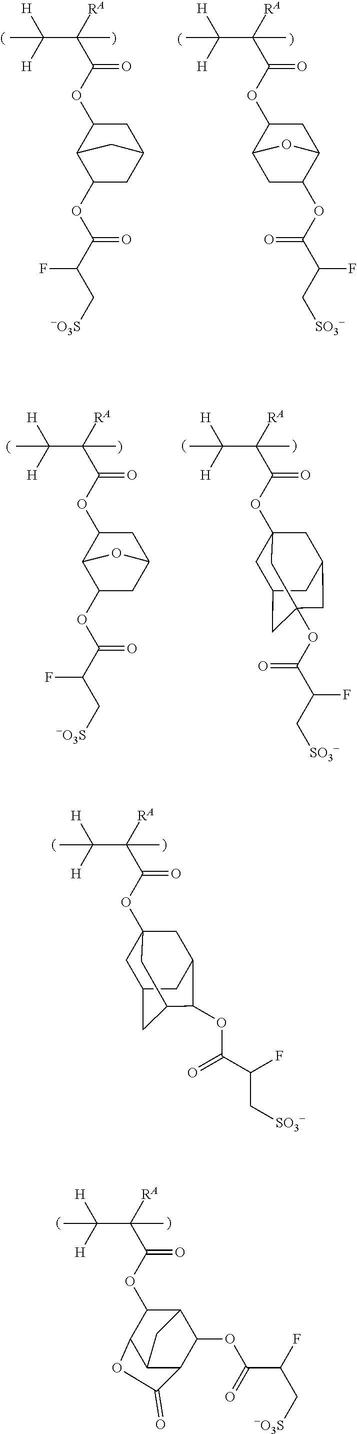

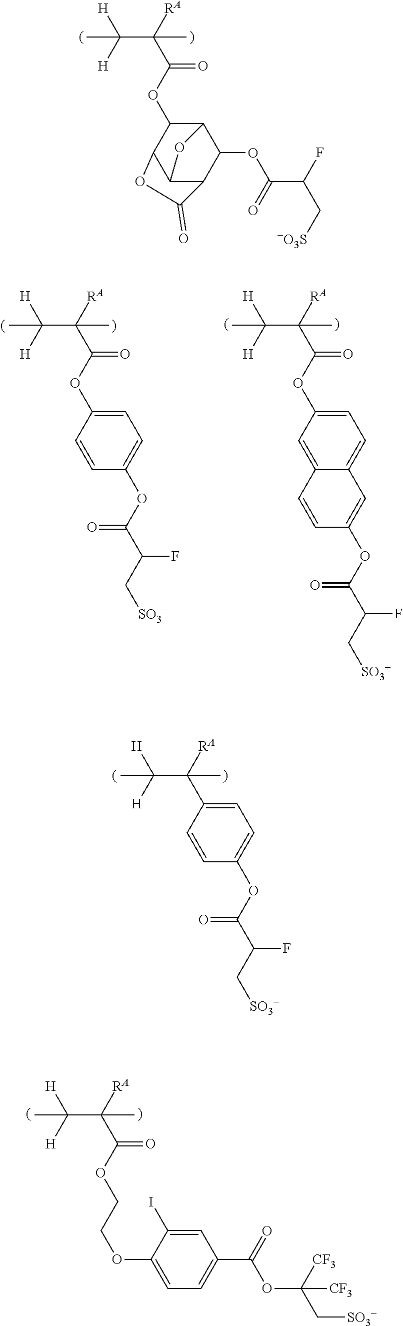

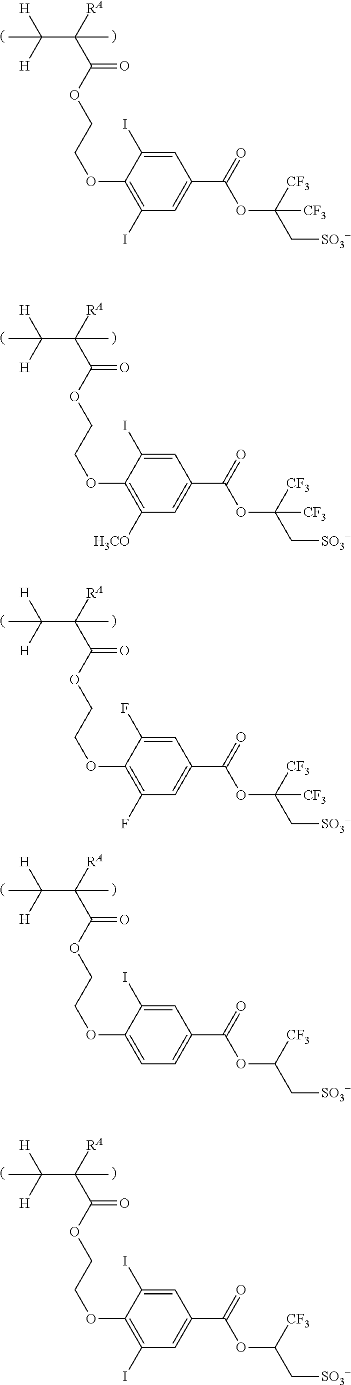

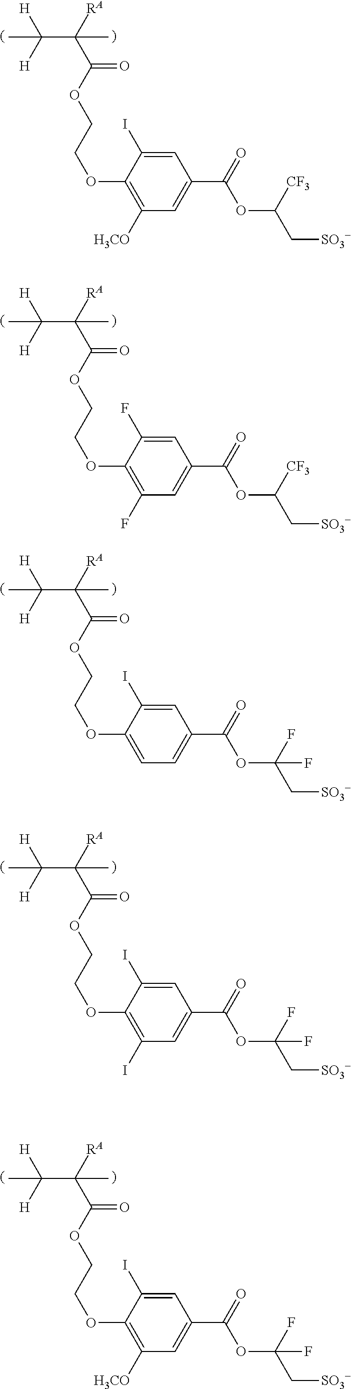

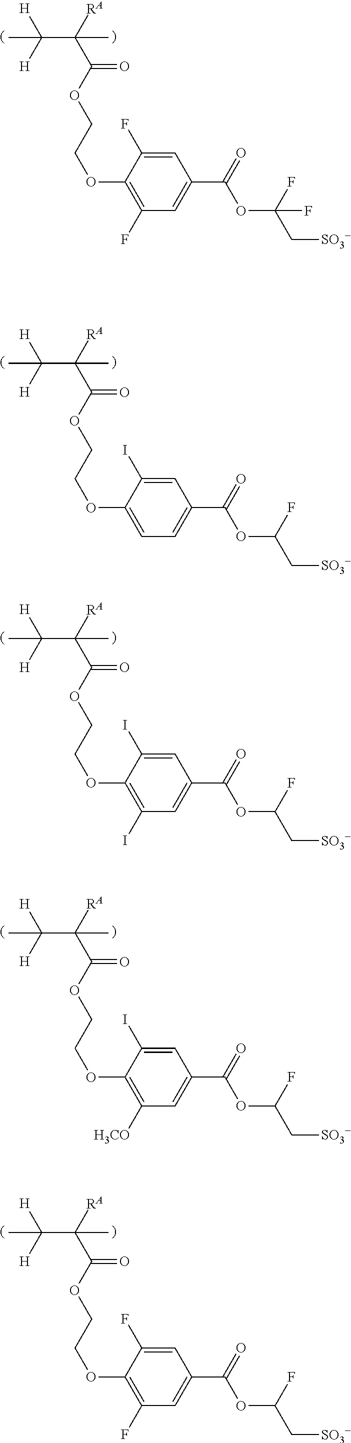

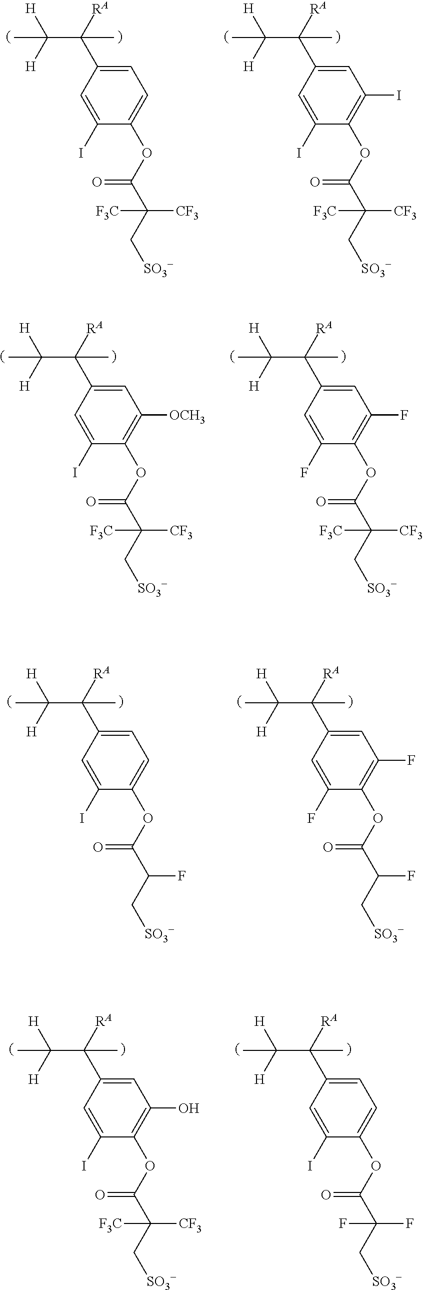

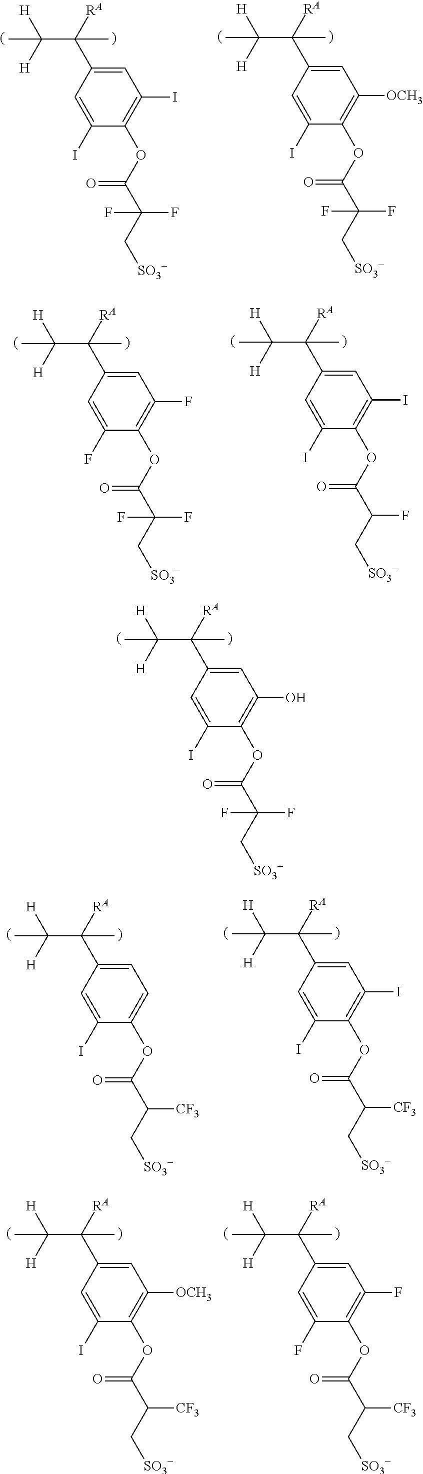

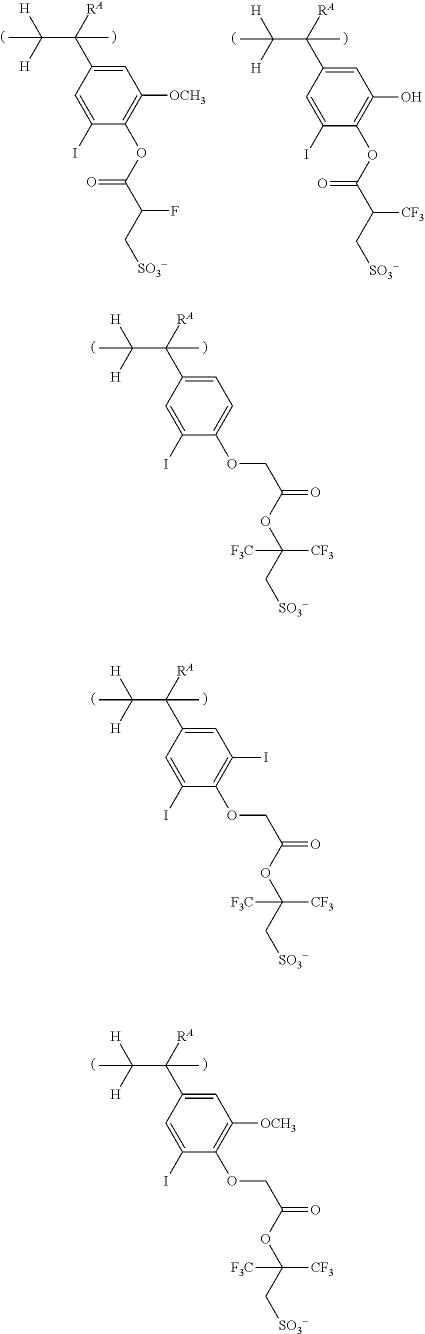

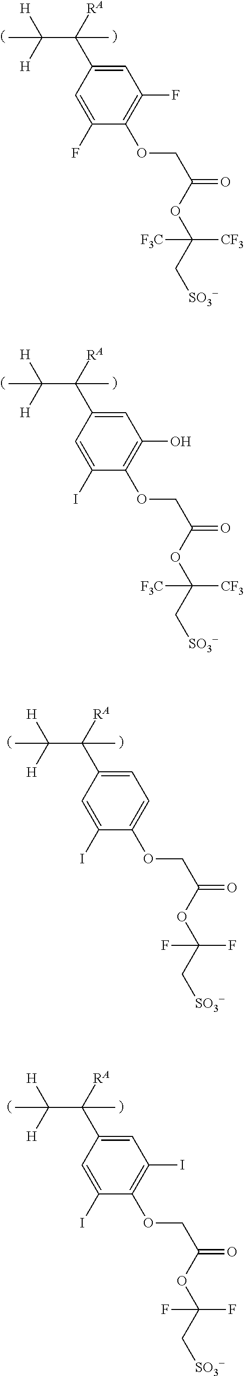

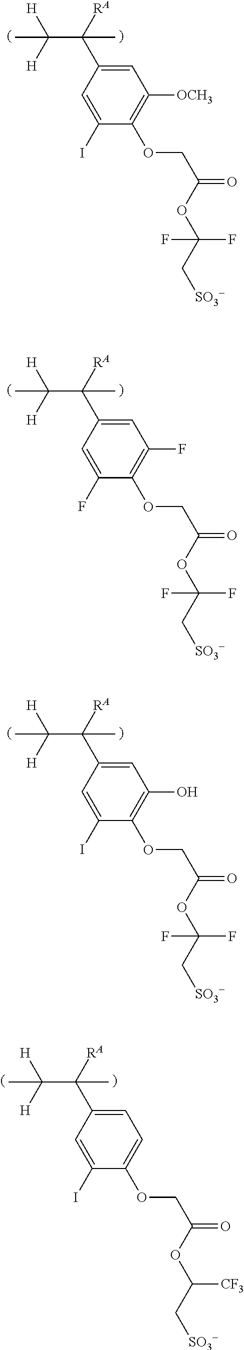

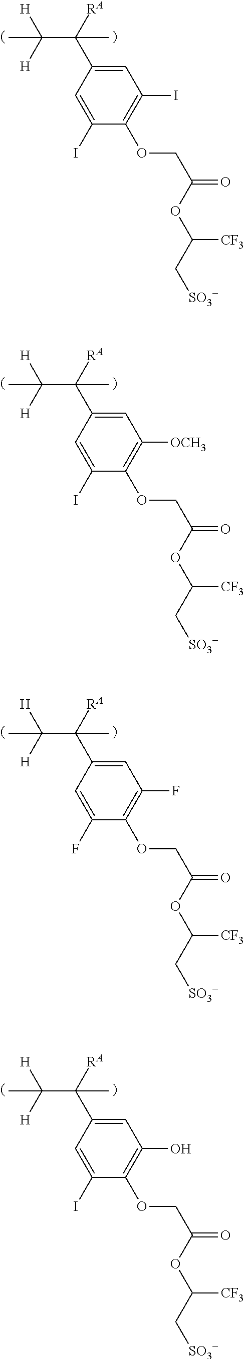

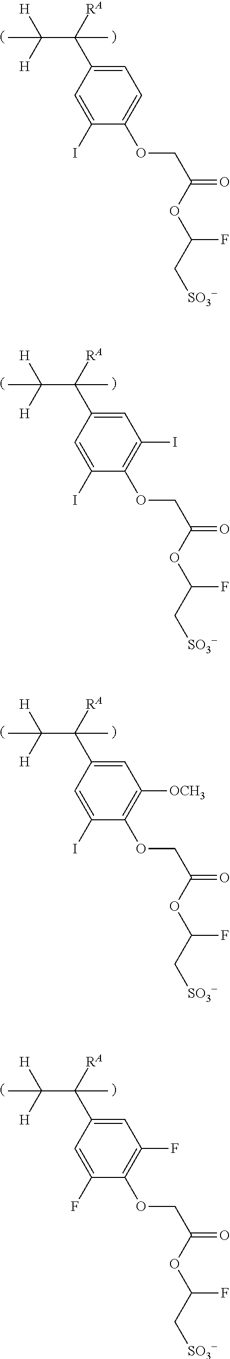

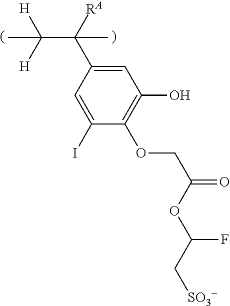

Examples of the anion in repeat unit c4 are shown below, but not limited thereto. Herein R^{A} is as defined above.

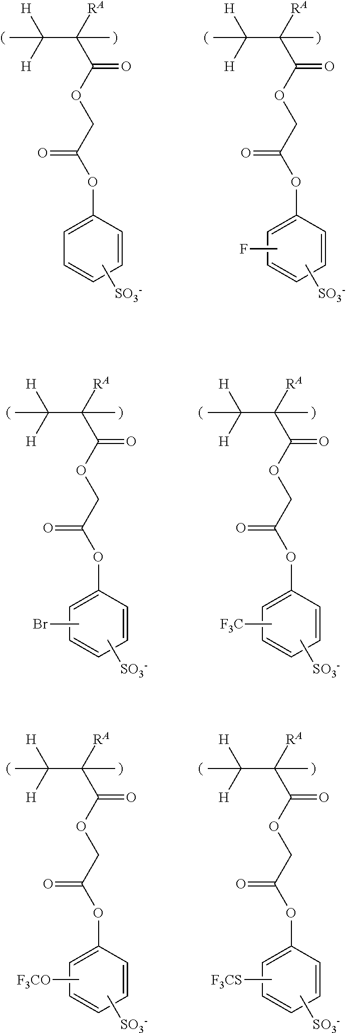

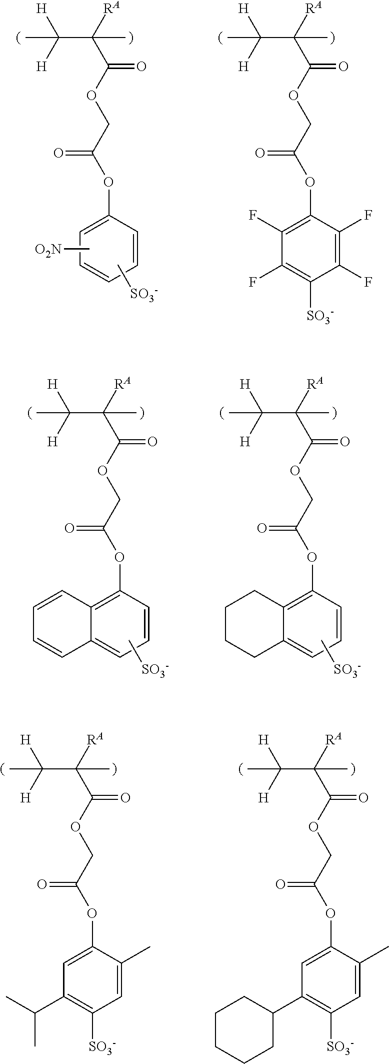

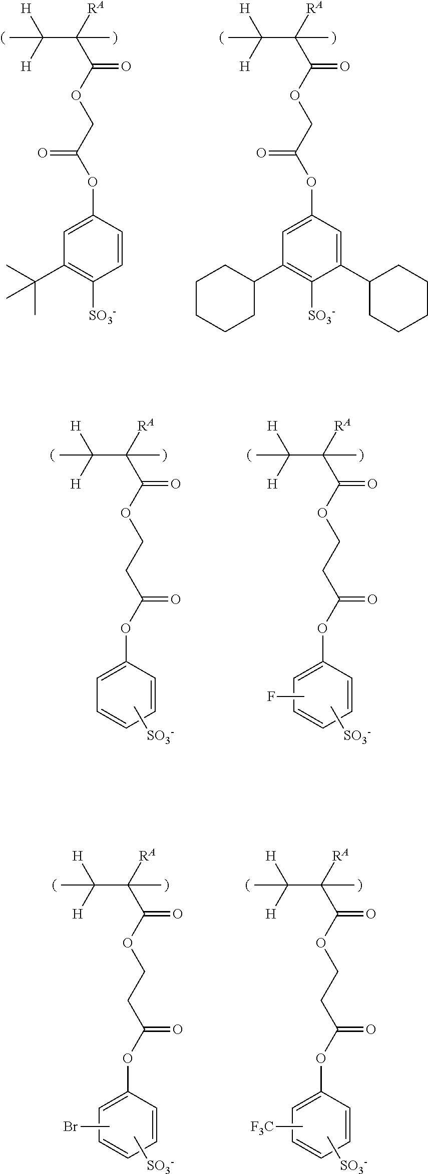

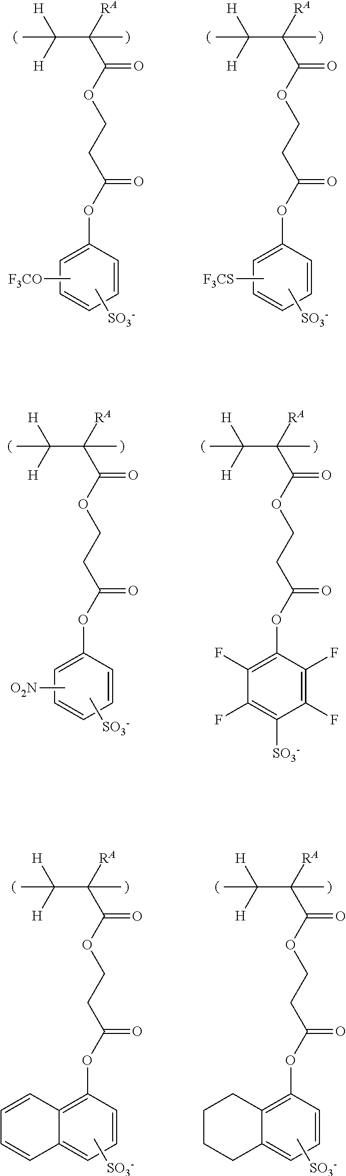

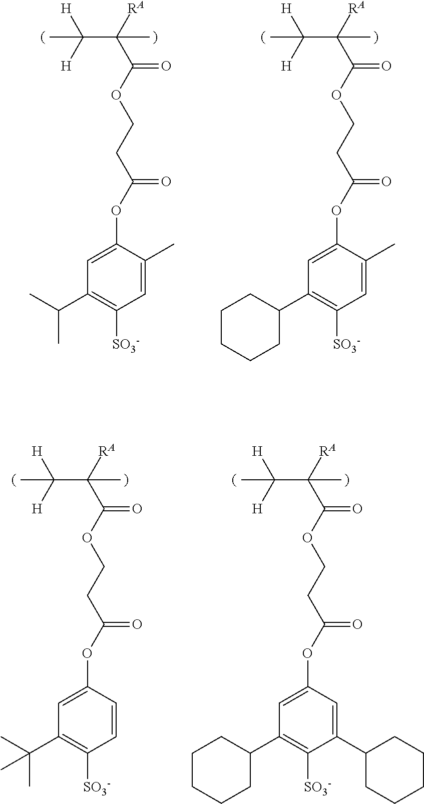

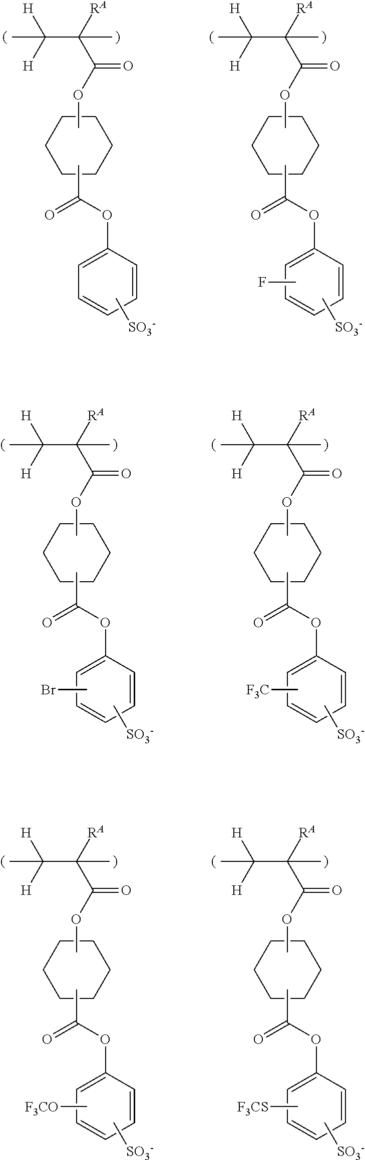

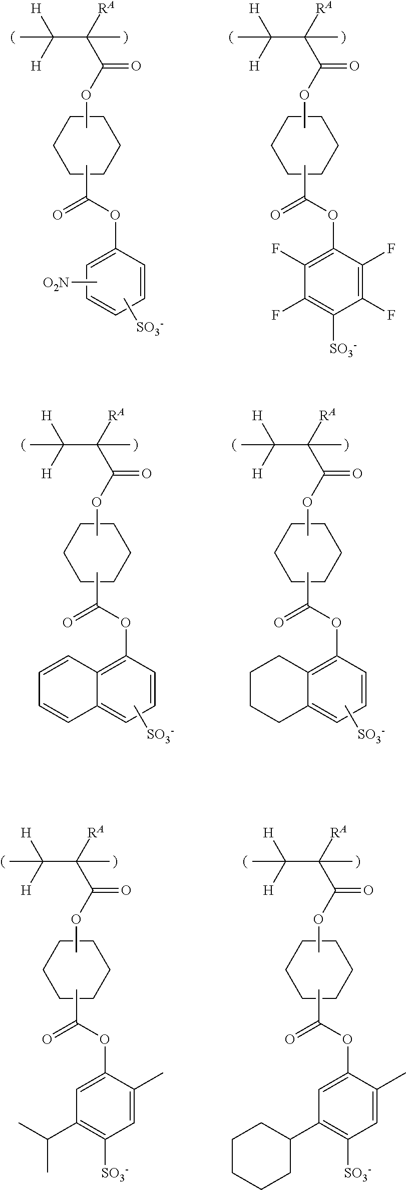

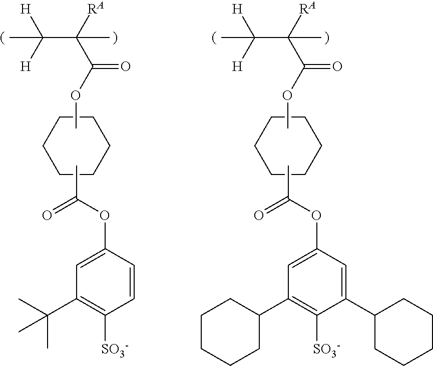

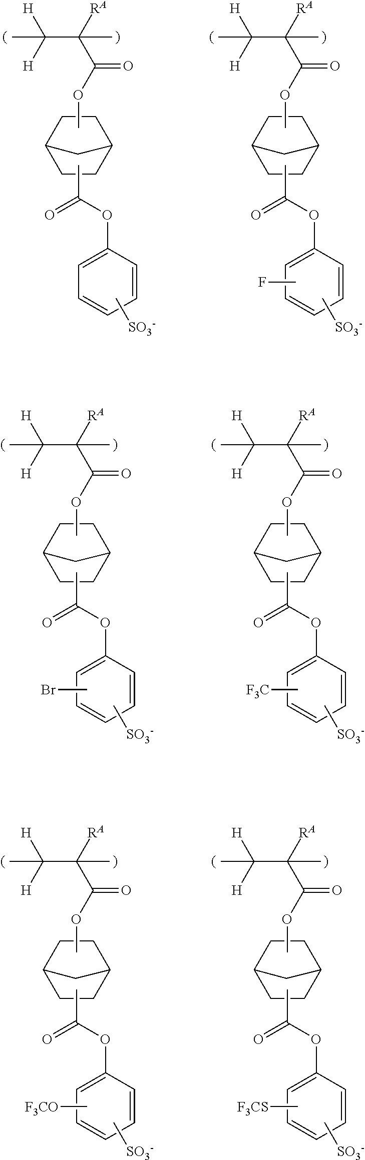

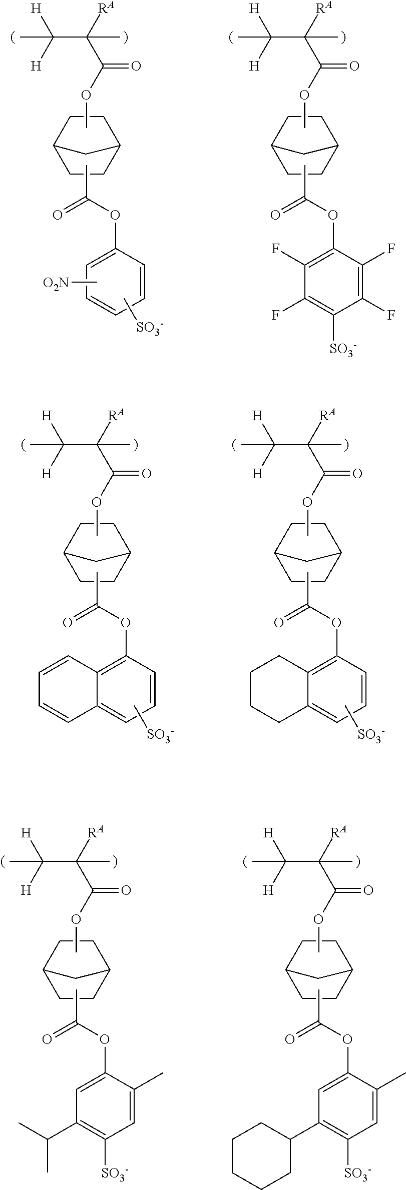

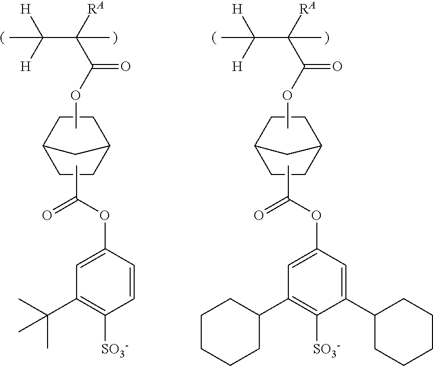

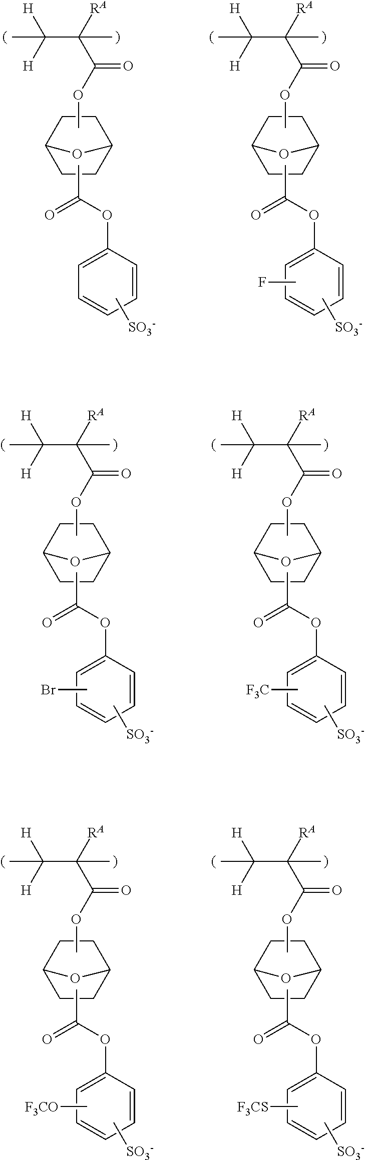

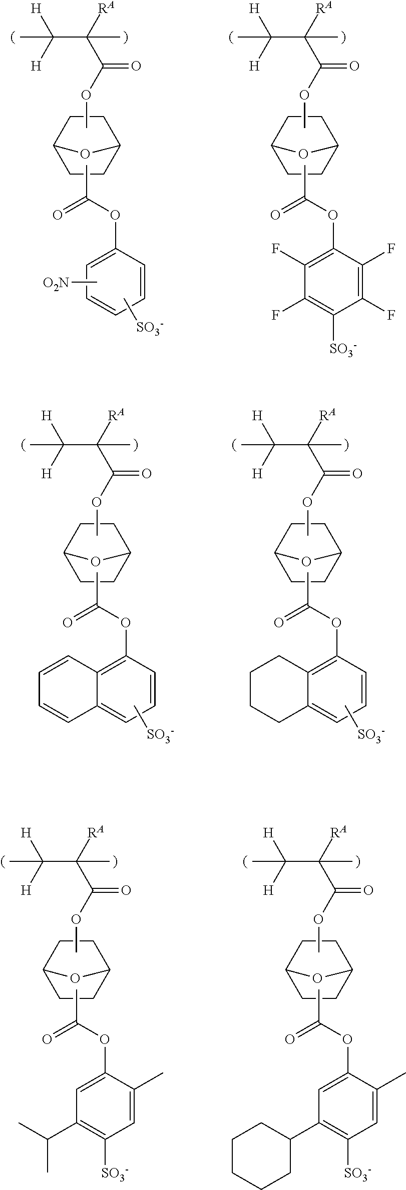

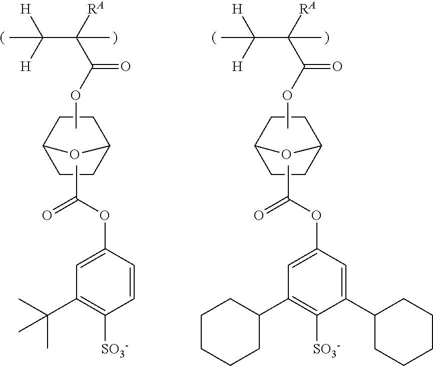

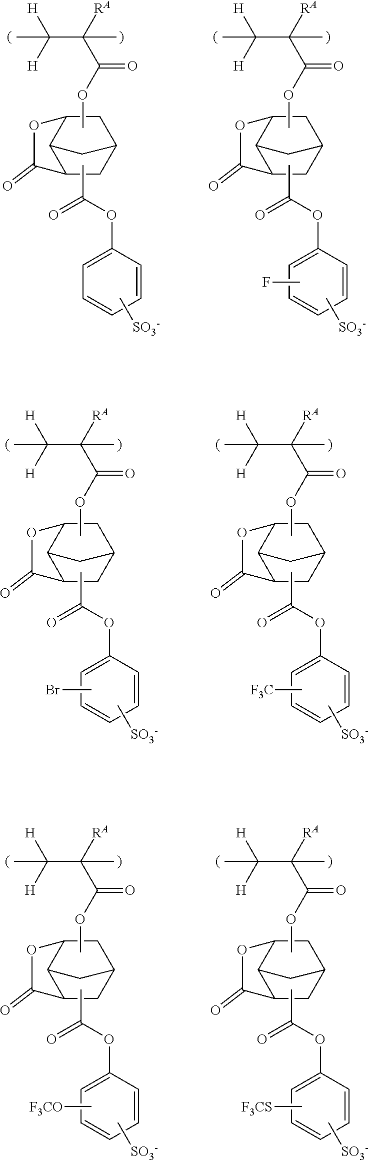

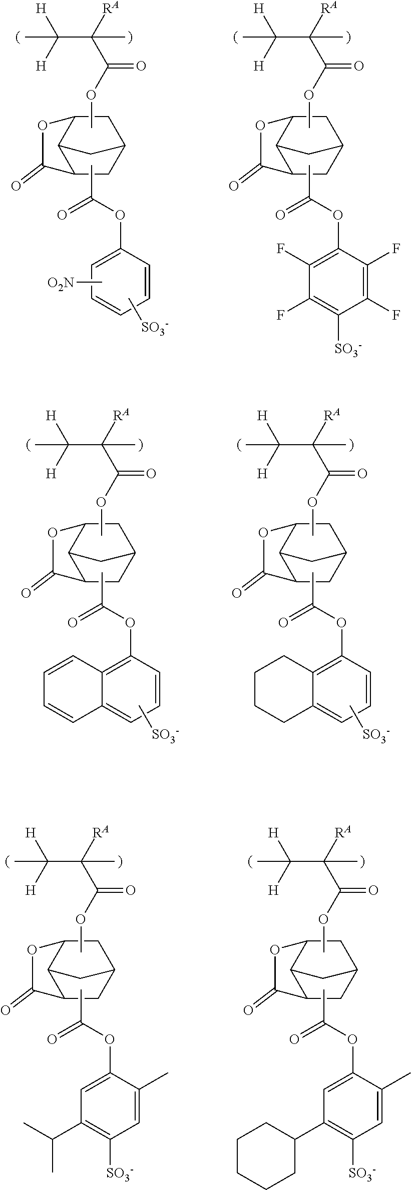

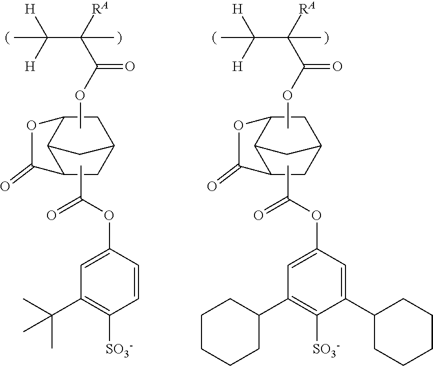

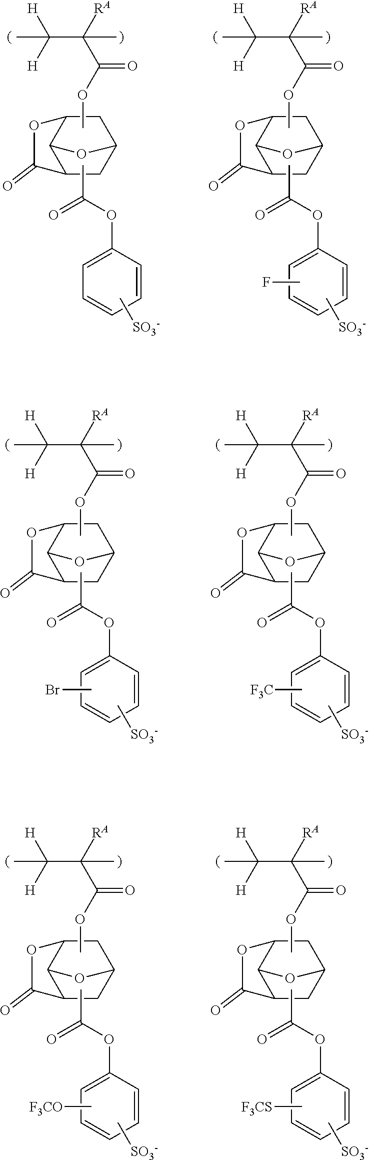

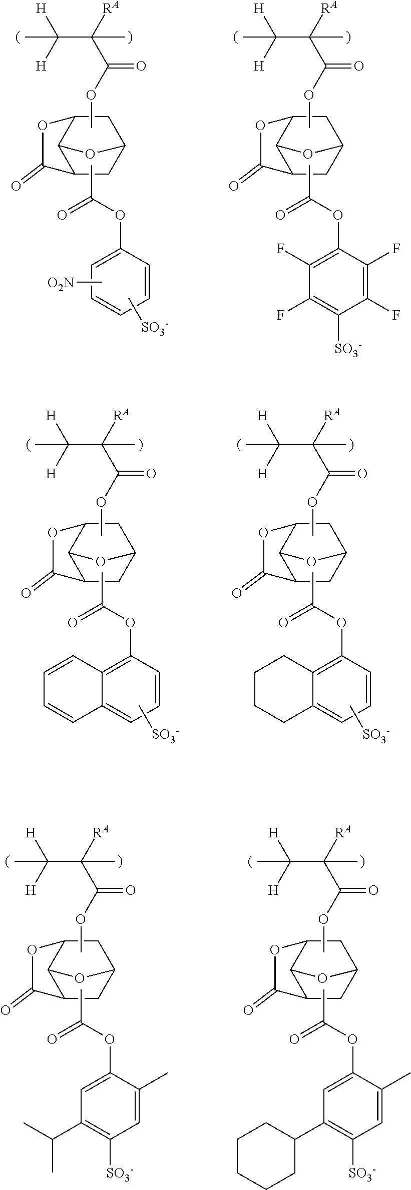

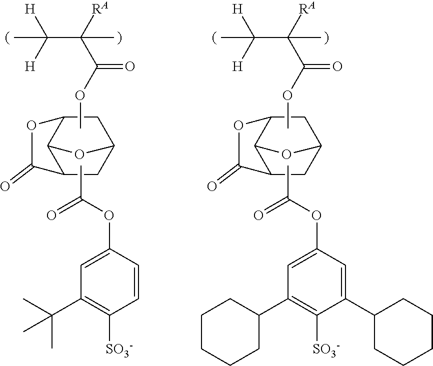

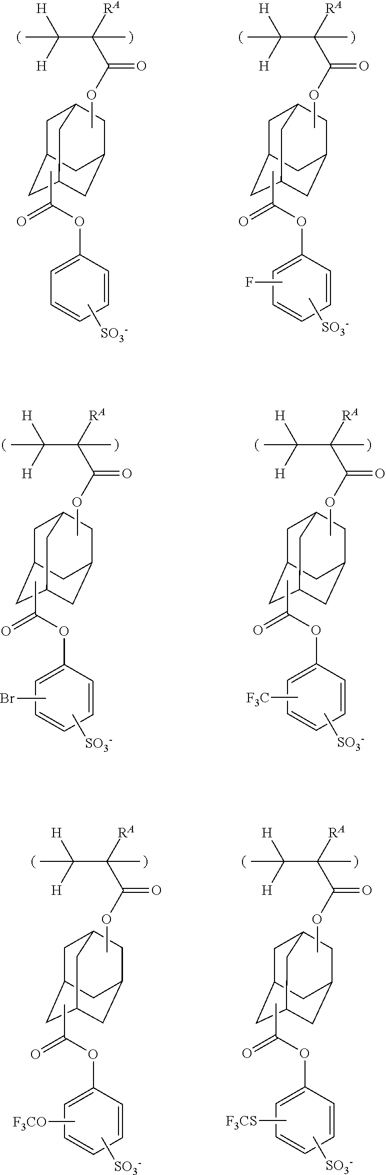

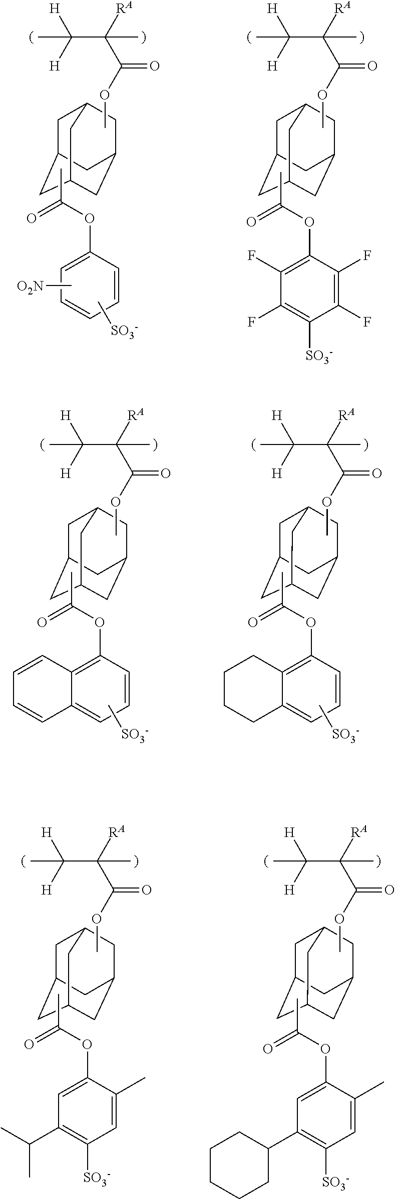

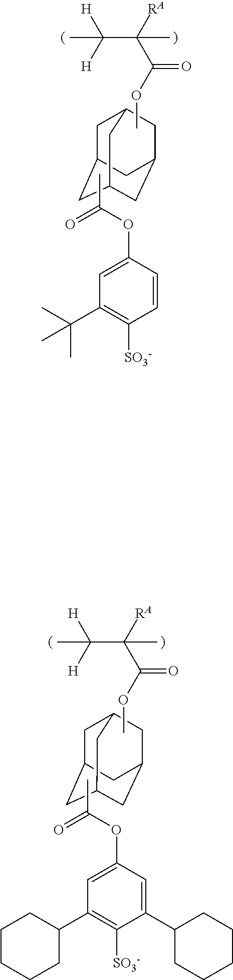

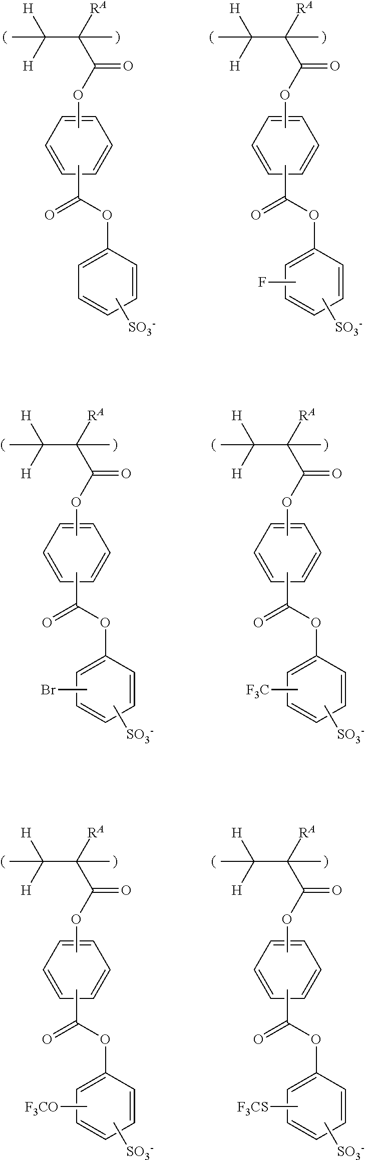

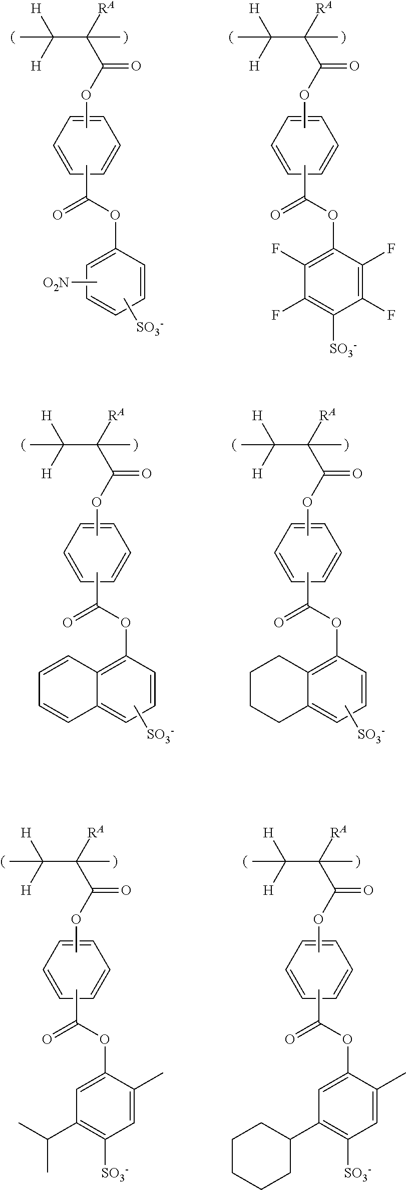

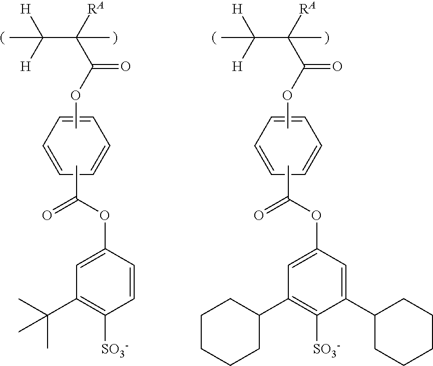

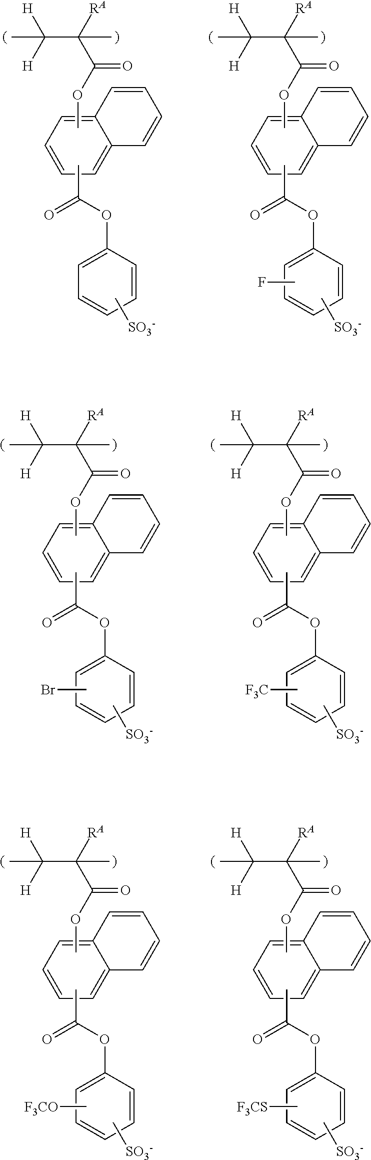

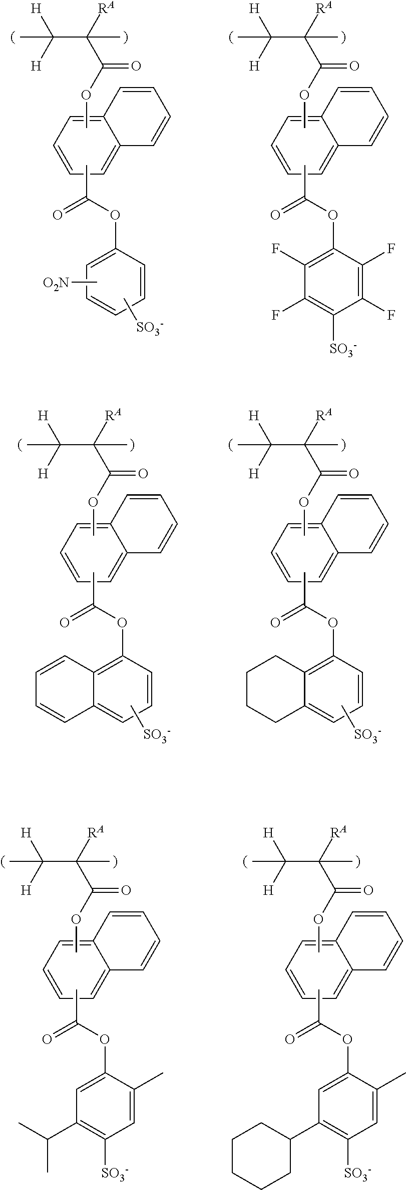

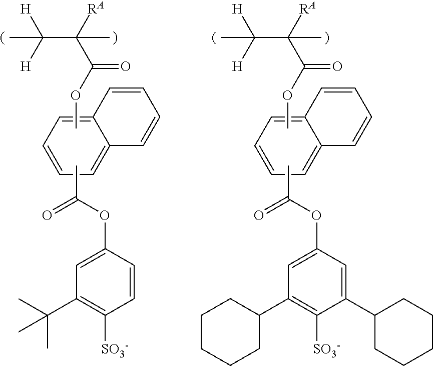

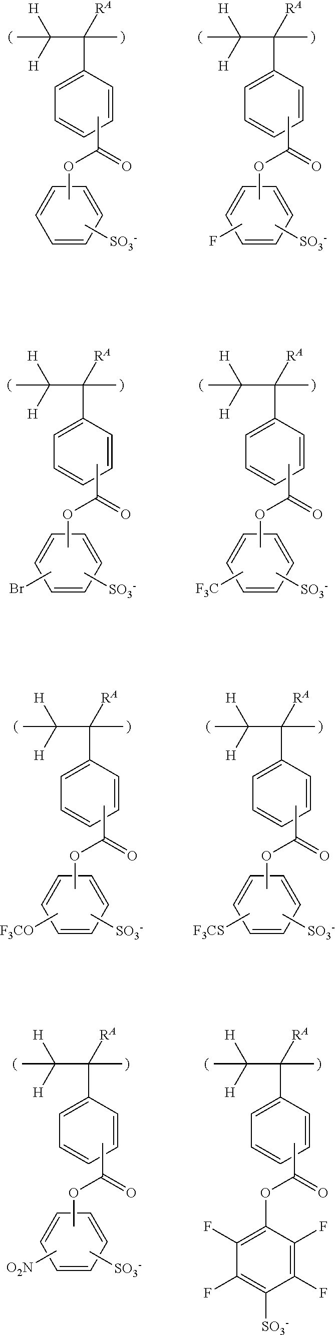

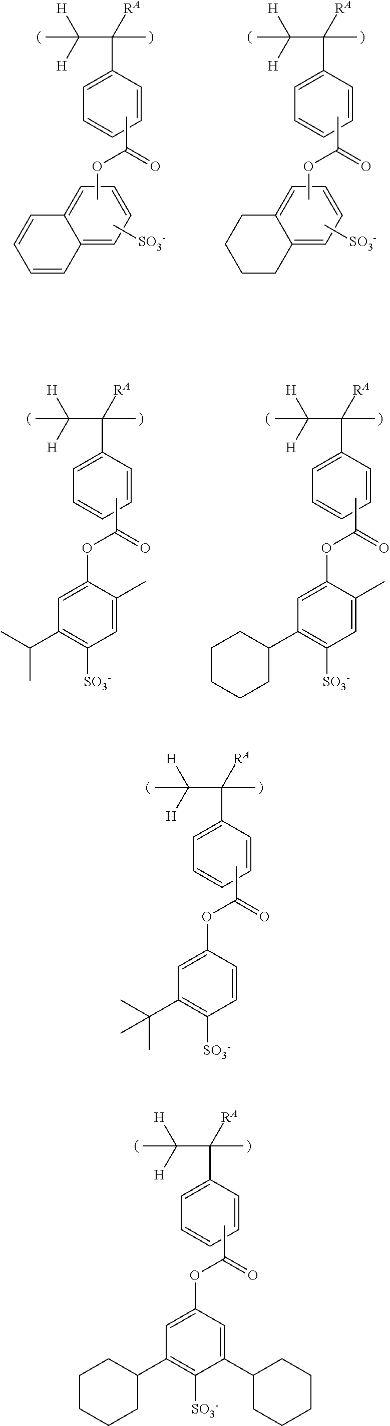

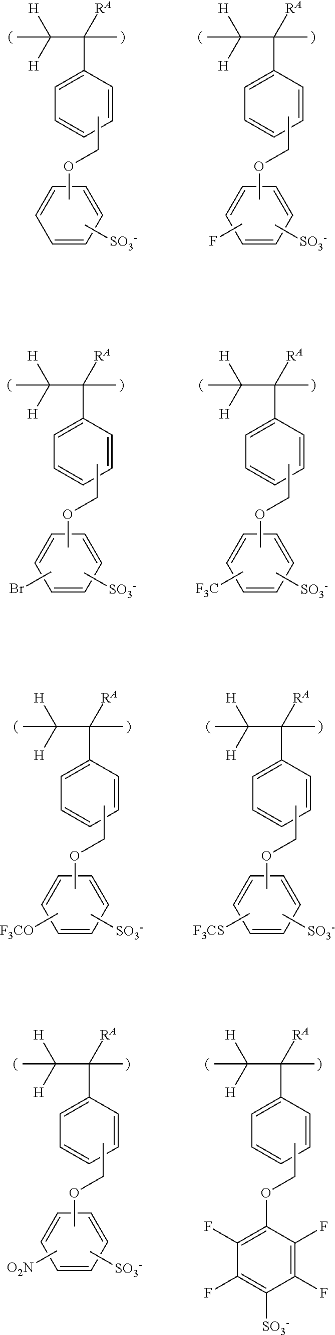

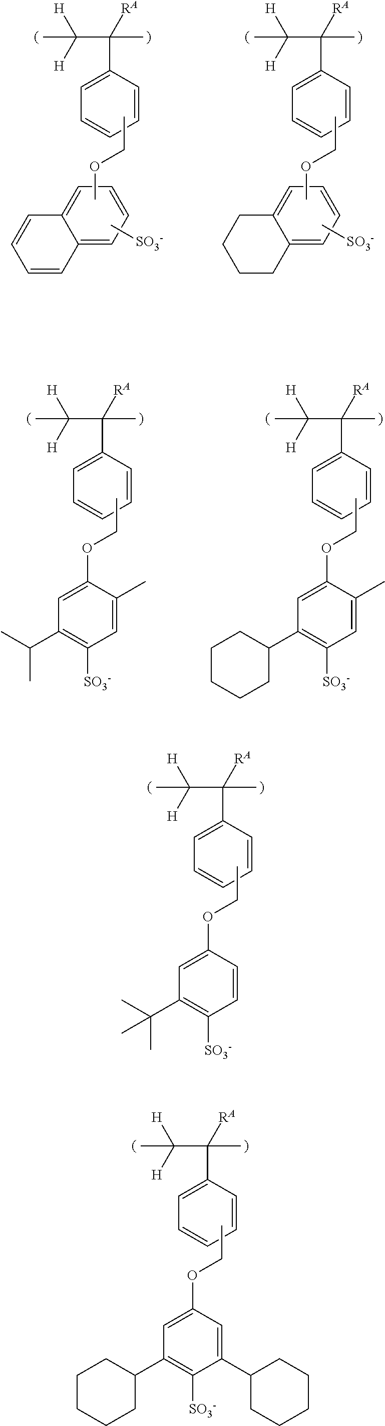

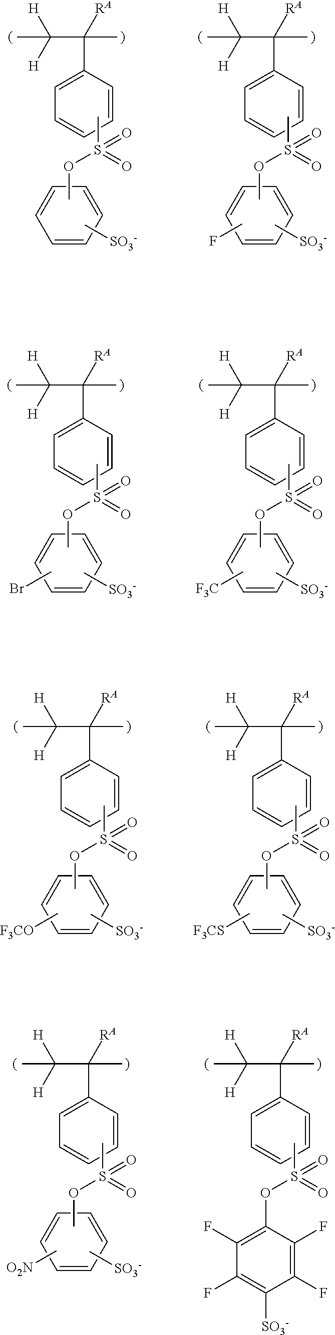

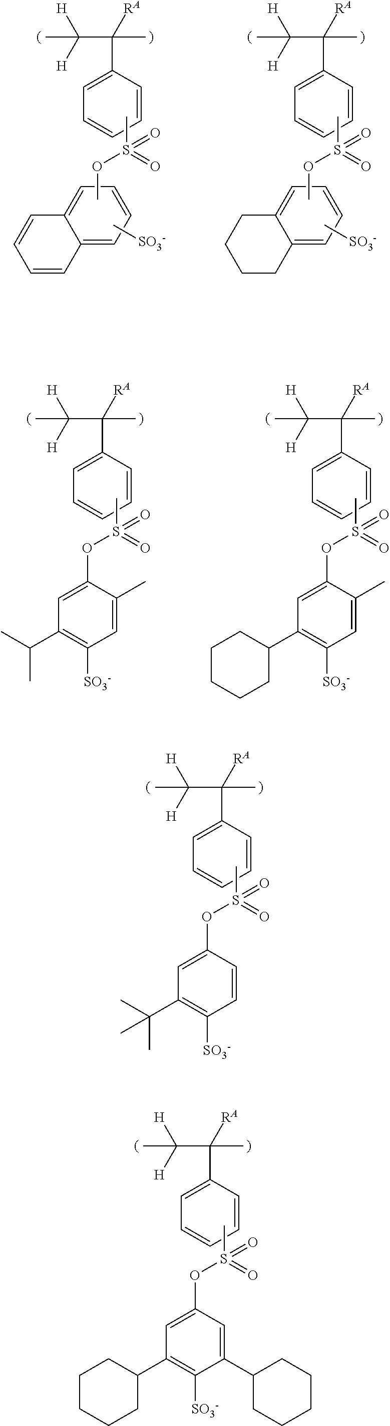

Examples of the anion in repeat unit c5 are shown below, but not limited thereto. Herein R^{A} is as defined above.

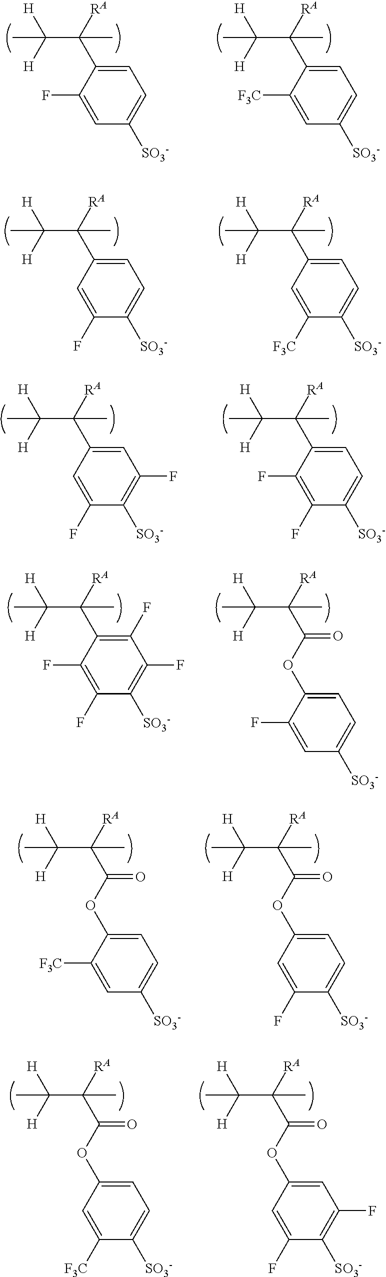

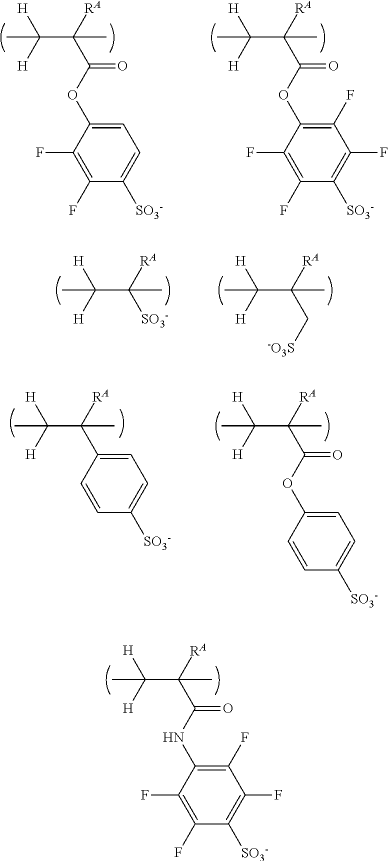

In formulae (c2) to (c5), A⁺ is an onium cation. Suitable onium cations include ammonium, sulfonium and iodonium cations, with the sulfonium and iodonium cations being preferred. Examples of the sulfonium cation are as described in JP-A 2024-003744, [0102]-[0125] and JP-A 2023-169812, [0070]-[0085], and include cations of formula (A2), but are not limited thereto. Examples of the iodonium cation are as described in JP-A 2024-000259, [0181], but not limited thereto. Examples of the ammonium cation are as exemplified for the ammonium cation of formula (am-1) described later, but not limited thereto.

Examples of repeat units (c1) to (c5) include arbitrary combinations of the anion with the cation, both as exemplified above.

Of the repeat units (c1) to (c5), repeat units (c2) to (c5) are preferred from the aspect of controlling acid diffusion. Repeat units (c2), (c4) and (c5) are more preferred from the aspect of the acid strength of generated acid. Repeat units (c2) are most preferred from the aspect of solvent solubility.

The base polymer may further comprise repeat units (d) of a structure having a hydroxy group protected with an acid labile group (hereinafter, also referred to repeat units (d)). The repeat unit (d) is not particularly limited as long as the unit includes one or more structures having a hydroxy group protected with a protective group such that the protective group is decomposed to generate the hydroxy group under the action of acid. Repeat units having the formula (d1) are preferred.

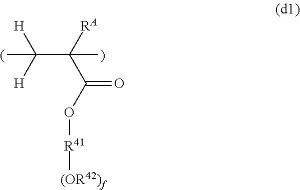

In formula (d1), R^{A} is as defined above. R⁴¹ is a C₁-C₃₀ (e+1)-valent hydrocarbon group which may contain a heteroatom. R⁴² is an acid labile group. The subscript f is 1, 2, 3 or 4.

In formula (d1), the acid labile group R⁴² is deprotected under the action of acid so that a hydroxy group is generated. The structure of R⁴² is not particularly limited. An acetal structure, ketal structure, alkoxycarbonyl group and alkoxymethyl group having the following formula (d2) are preferred, with the alkoxymethyl group having formula (d2) being more preferred.

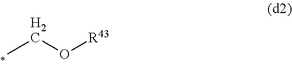

Herein * designated a point of attachment. R⁴³ is a C₁-C₁₅ hydrocarbyl group.

Illustrative examples of the acid labile group R⁴², the alkoxymethyl group having formula (d2), and the repeat units (d) are as exemplified for the repeat units (d) in JP-A 2020-111564 (US 20200223796).

In addition to the foregoing units, the base polymer may further comprise repeat units (e) derived from indene, benzofuran, benzothiophene, acenaphthylene, chromone, coumarin, norbornadiene and derivatives thereof. Examples of the monomer from which repeat units (e) are derived are shown below, but not limited thereto.

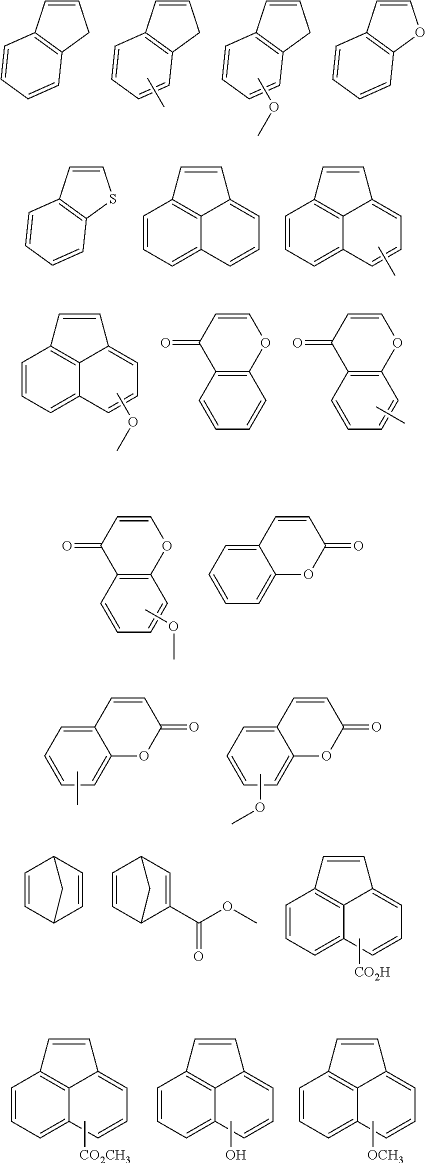

The base polymer may further comprise repeat units (f) derived from indane, vinylpyridine or vinylcarbazole.

In the polymer, repeat units (a1), (a2), (a3), (b1), (b2), (c1), (c2), (c3), (c4), (c5), (d), (e) and (f) are incorporated in a ratio of preferably 0≤a1≤0.8, 0≤a2≤0.8, 0≤a3≤0.6, 0≤b1≤0.8, 0≤b2≤0.5, 0≤c1≤0.4, 0≤c2≤0.4, 0≤c3≤0.4, 0≤c4≤0.4, 0≤c5≤0.4, 0≤d≤0.3, 0≤e≤0.3, and 0≤f≤0.3; more preferably 0≤a1≤0.7, 0≤a2≤0.7, 0≤a3≤0.5, 0≤b1≤0.7, 0≤b2≤0.4, 0≤c1≤0.3, 0≤c2≤0.3, 0≤c3≤0.3, 0≤c4≤0.3, 0≤c5≤0.3, 0≤d≤0.2, 0≤e≤0.2, and 0≤f≤0.2. It is noted that a1+a2+a3+b1+b2+c1+c2+c3+c4+c5+d+e+f≤1.0.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. In the invention, Mw is a value measured by gel permeation chromatography (GPC) with THF or N,N-dimethylformamide (DMF) as a solvent, and calculated as polystyrene.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. A Mw/Mn in the range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Examples of the organic solvent which can be used for polymerization include toluene, benzene, tetrahydrofuran THF, diethyl ether, dioxane, cyclohexane, cyclopentane, methyl ethyl ketone (MEK), propylene glycol monomethyl ether acetate (PGMEA), and γ-butyrolactone (GBL). Examples of the polymerization initiator used herein include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, and more preferably 2 to 12 hours from the viewpoint of production efficiency.

The polymerization initiator may be added to the monomer solution before supply to a reaction vessel, or an initiator solution may be prepared separately from the monomer solution and each solution may be supplied to a reaction vessel independently. Since there is a possibility that the initiator generates a radical in the standby time, by which polymerization reaction takes place to form an ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, hydroxystyrene or hydroxyvinylnaphthalene and other monomers may be dissolved in an organic solvent, a radical polymerization initiator is added thereto, and the mixture is heated for polymerization. Instead, as alternative method, acetoxystyrene or acetoxyvinylnaphthalene may be used and after polymerization, the acetoxy group is deprotected by alkaline hydrolysis, for thereby converting the polymer product to polyhydroxystyrene or hydroxypolyvinylnaphthalene.

For alkaline hydrolysis, a base such as aqueous ammonia or triethylamine may be used. Preferably the reaction temperature is −20° C. to 100° C., more preferably 0° C. to 60° C. The reaction time is 0.2 to 100 hours, more preferably 0.5 to 20 hours.

The amount of each monomer in the monomer solution is to be appropriately set, for example, so as to achieve the foregoing preferred content ratio of the recurring unit.

The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145]. Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtration of an amplified resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (B) may be used alone or as a blend of two or more polymers which differ in compositional ratio, Mw and/or Mw/Mn. Component (B) may also be a blend of the base polymer defined above and a hydrogenated product of ROMP. For the ROMP, reference is made to JP-A 2003-66612.

### (C) Organic Solvent

The chemically amplified resist composition of the invention may comprise an organic solvent as component (C). The organic solvent (C) is not particularly limited as long as the foregoing and other components are soluble therein. Examples of the organic solvent include ketones such as cyclopentanone, cyclohexanone and methyl-2-n-pentyl ketone; alcohols such as 3-methoxybutanol, 3-methyl-3-methoxybutanol, 1-methoxy-2-propanol, 1-ethoxy-2-propanol; keto-alcohols such as DAA, ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as GBL, which may be used alone or in admixture.

Of the foregoing organic solvents, it is recommended to use 1-ethoxy-2-propanol, PGMEA, cyclohexanone, GBL, DAA and mixtures thereof because the base polymer (B) is most soluble therein.

The content of the organic solvent (C) in the chemically amplified resist composition of the invention is preferably 200 to 5,000 parts by weight, more preferably 400 to 3,500 parts by weight per 80 parts by weight of the base polymer (B). The organic solvent (C) may be used alone or in admixture.

### (D) Photoacid Generator

The chemically amplified resist composition of the invention may further comprise (D) a photoacid generator (PAG). The PAG is not particularly limited as long as it is capable of generating an acid having a higher acid strength than the sulfonic acid generated by the quencher of component (A), upon exposure to high-energy radiation.

The preferred PAG is a salt having the formula (2) or (3).

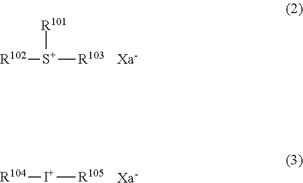

In formula (2), R¹⁰¹ to R¹⁰⁵ are each independently a C₁-C₂₀ hydrocarbyl group which may contain a heteroatom. Any two of R¹⁰¹ and R¹⁰² and R¹⁰³ may bond together to form a ring with a sulfur atom to which they are attached. Examples of the hydrocarbyl group are as exemplified above for the hydrocarbyl groups R¹ to R⁴ in formula (1).

Examples of the cation in the sulfonium salt having formula (2) are as described in JP-A 2024-003744, paragraphs [0102]-[0125] and JP-A 2023-169812, paragraphs [0070]-[0085], and include cations in the sulfonium salts of formula (1). Examples of the cation in the iodonium salt having formula (3) are as described in JP-A 2024-000259, paragraph [0181].

In formulae (2) and (3), Ma⁻ is an anion of a strong acid. Examples of the strong acid anion are any of anions of the formulae (c1-1) to (c1-5).

Photoacid generators (D) having the formula (4) are also preferred.

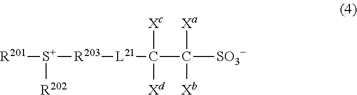

In formula (4), R²⁰¹ to R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹ and R²⁰² and R²⁰³ may bond together to form a ring with a sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, tert-pentyl, n-pentyl, n-hexyl, n-octyl, 2-ethylhexyl, n-nonyl and n-decyl groups; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl and adamantyl groups; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl and anthracenyl groups; and combinations thereof. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl and heptadecane-1,17-diyl groups; C₃-C₃₀ cyclic saturated hydrocarbylene groups such as cyclopentanediyl, cyclohexanediyl, norbornanediyl and adamantanediyl groups; and arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene groups. In the hydrocarbylene group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. The heteroatom is preferably oxygen.

In formula (4), L²¹ is a single bond, ether bond, or C₁-C₂₀ hydrocarbylene group which may contain a heteroatom. The hydrocarbylene group may be saturated or unsaturated and straight, branched or cyclic. Examples of the hydrocarbylene group are as exemplified above for hydrocarbylene group R²⁰³.

In formula (4), X^{a}, X^{b}, X^{c} and X^{d} are each independently hydrogen, fluorine or trifluoromethyl. It is noted that at least one of X^{a}, X^{b}, X^{c} and X^{d} is fluorine or trifluoromethyl.

Preferably, the photoacid generator of formula (4) has the formula (4′).

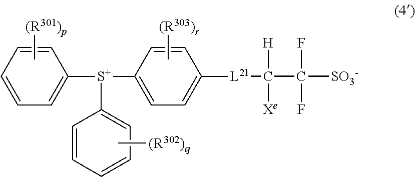

In formula (4′), L²¹ is as defined above. R^{HF} is hydrogen or trifluoromethyl, preferably trifluoromethyl. R³⁰¹, R³⁰² and R³⁰³ are each independently halogen or a C₁-C₂₀ hydrocarbyl group which may contain a heteroatom. The hydrocarbyl group may be saturated or unsaturated, and may be straight, branched, or cyclic. Examples thereof are as exemplified above for the hydrocarbyl group R^{fa1} in formula (c1-1-1). The subscripts p and q are each independently 0, 1, 2, 3, 4 or 5, and the subscript r is 0, 1, 2, 3 or 4.

Examples of the photoacid generator having formula (4) are as exemplified as for the photoacid generator having formula (2) in JP-A 2017-26980.

Of the foregoing photoacid generators, those having an anion of formula (c1-1-1) or (c1-4) are especially preferred because of reduced acid diffusion and high solubility in the resist solvent. Also those having formula (4′) are especially preferred because of extremely reduced acid diffusion.

When used, the PAG (D) is preferably added in an amount of 0.1 to 40 parts, and more preferably 0.5 to 20 parts by weight per 80 parts by weight of the base polymer (B). As long as the amount of the PAG (D) is in the range, good resolution is achievable and the risk of foreign matter being formed after development or during stripping of resist film is avoided. The PAG (D) may be used alone or in admixture.

### (E) Other Quencher

The chemically amplified resist composition of the invention may further comprise (E) a quencher other than component (A) (hereinafter, also referred to as the other quencher).

Onium salts having the formulae (5) and (6) are useful as the other quencher (E).

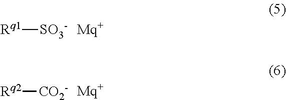

In formula (5), R^{q1} is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In formula (6), R^{q2} is hydrogen, or a C₁-C₄₀ hydrocarbyl group which may contain a heteroatom.

Examples of the C₁-C₄₀ hydrocarbyl group R^{q1} include C₁-C₄₀ alkyls such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl and n-decyl groups; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2.6}]decyl and adamantyl groups; and C₆-C₄₀ aryl groups such as phenyl, naphthyl and anthracenyl groups. In the hydrocarbyl group, some or all hydrogen may be substituted by a moiety containing a heteroatom such as oxygen, sulfur, nitrogen or halogen, and some —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonate ester bond, carbonate bond, lactone ring, sultone ring, carboxylic anhydride (—C(═O)—O—C(═O)—) or haloalkyl moiety.

Examples of the hydrocarbyl group R^{q2} include those exemplified above for R^{q1}, fluorinated saturated hydrocarbyl groups such as trifluoromethyl and trifluoroethyl groups, and fluorinated aryl groups such as pentafluorophenyl and 4-trifluoromethylphenyl groups.

Examples of the anion in the onium salt having formula (5) are shown below, but not limited thereto.

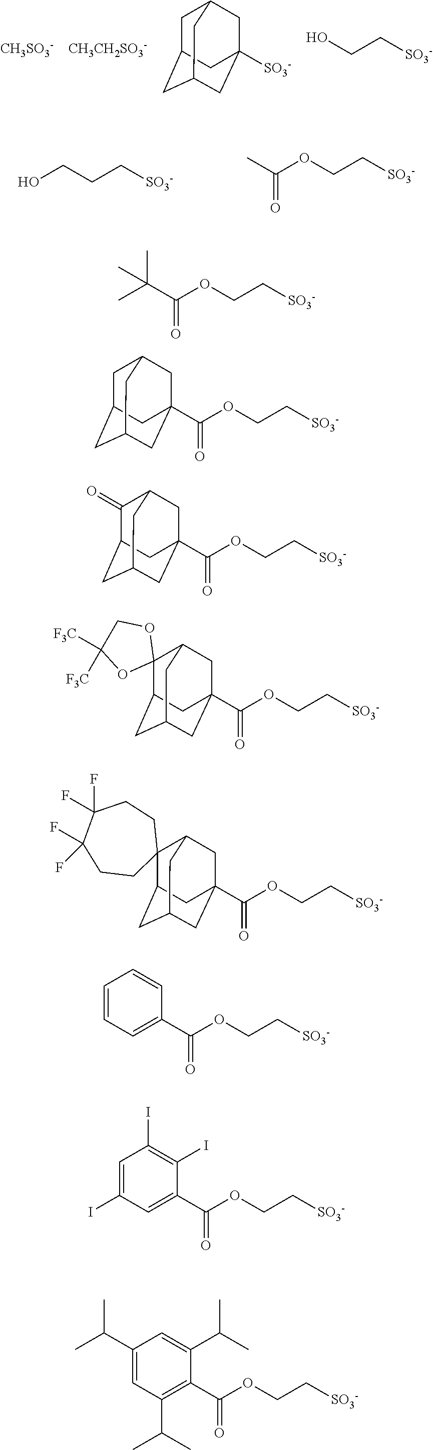

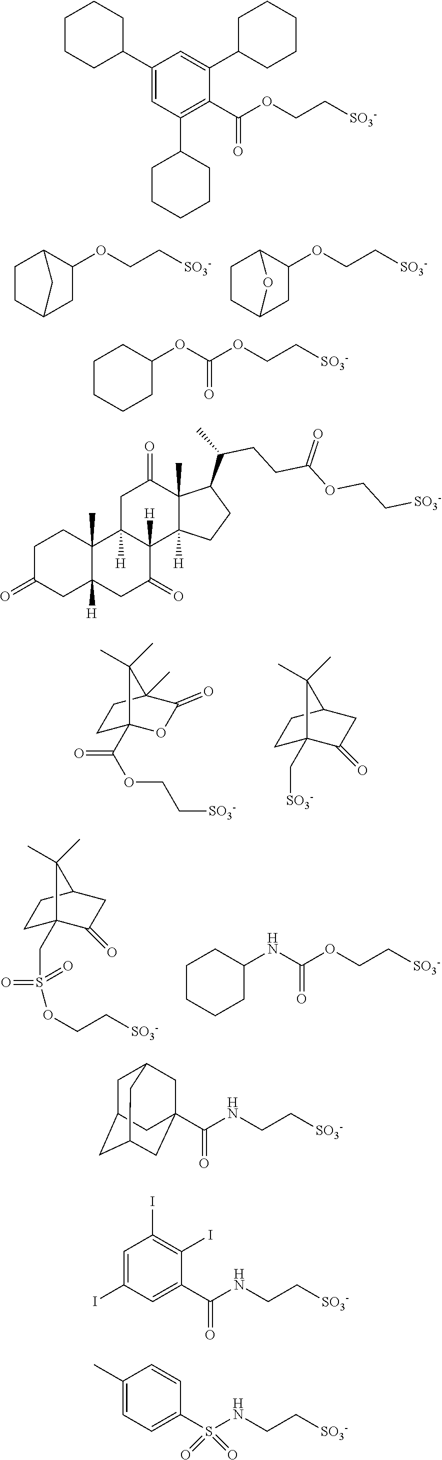

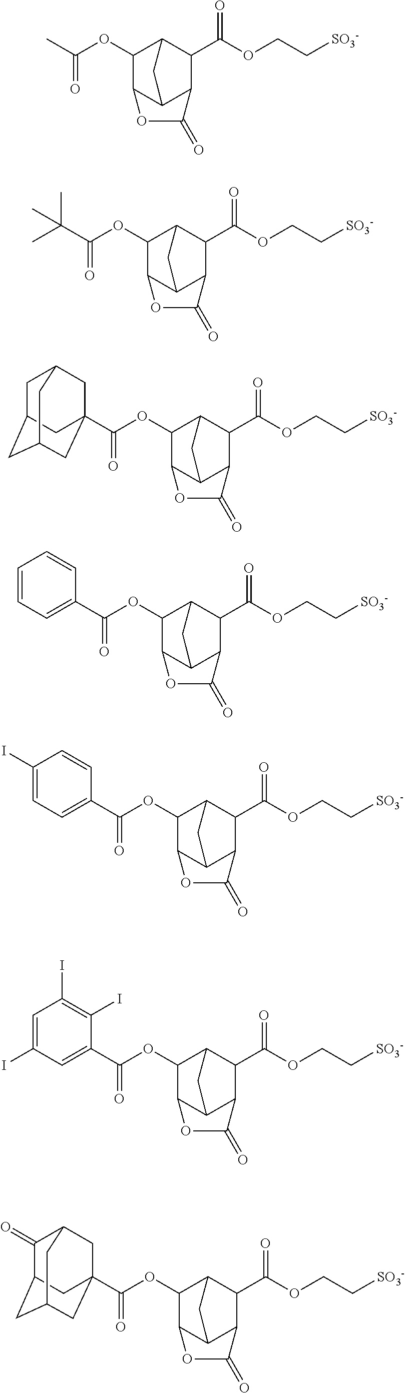

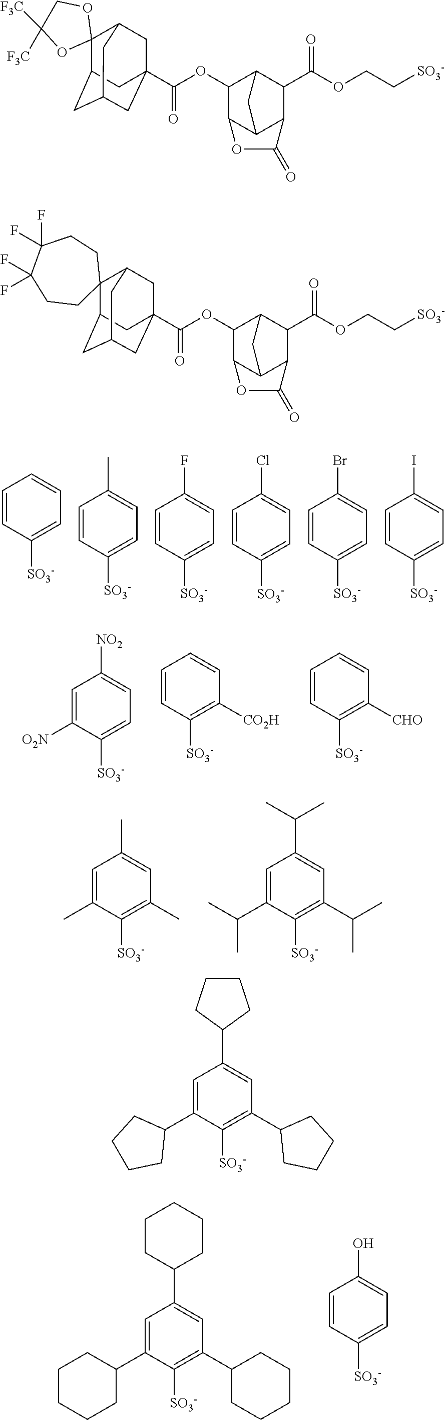

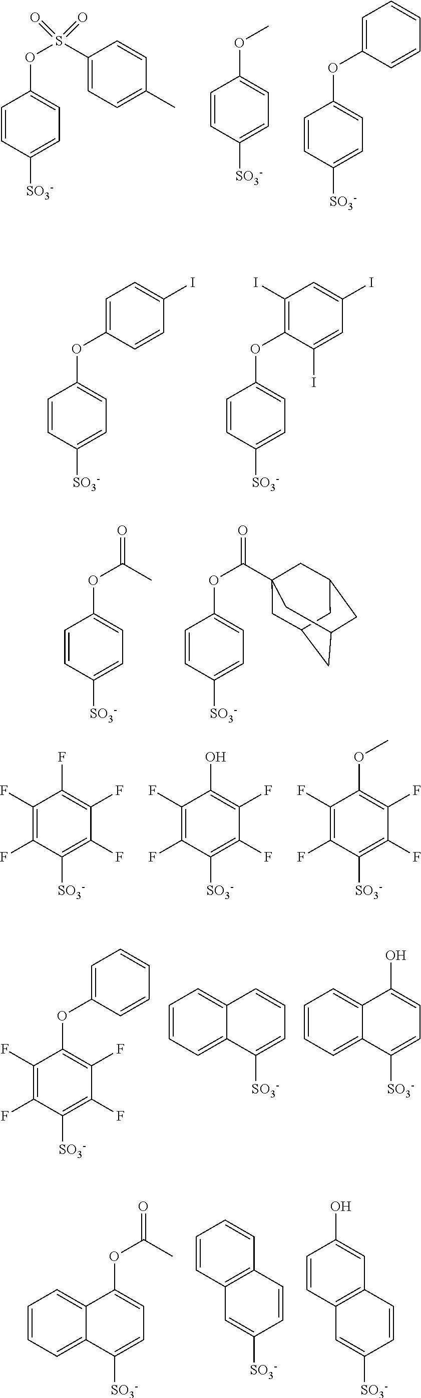

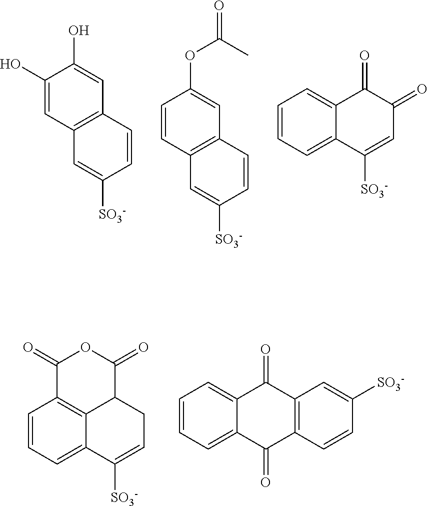

Examples of the anion in the onium salt having formula (6) are shown below, but not limited thereto.

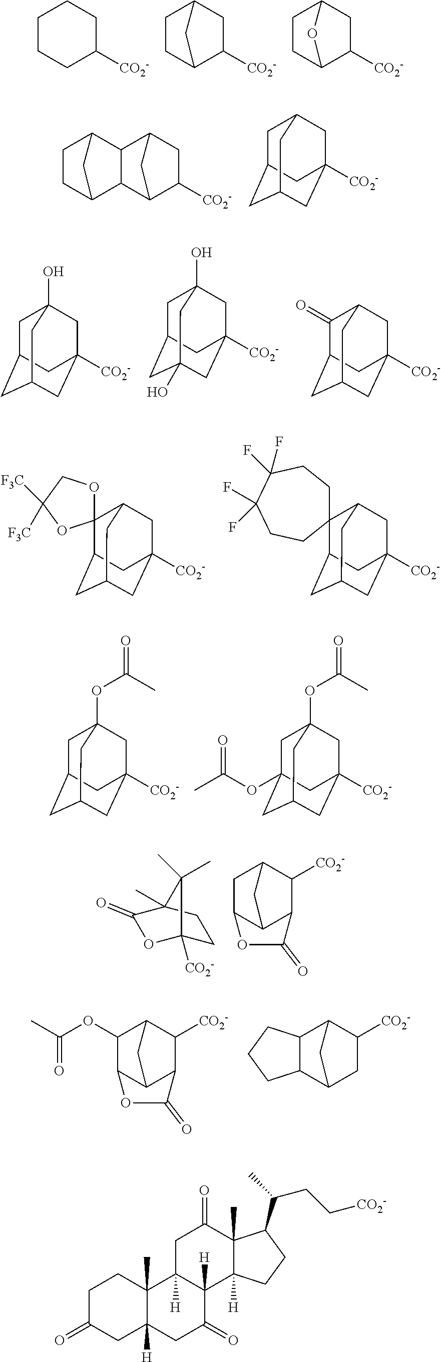

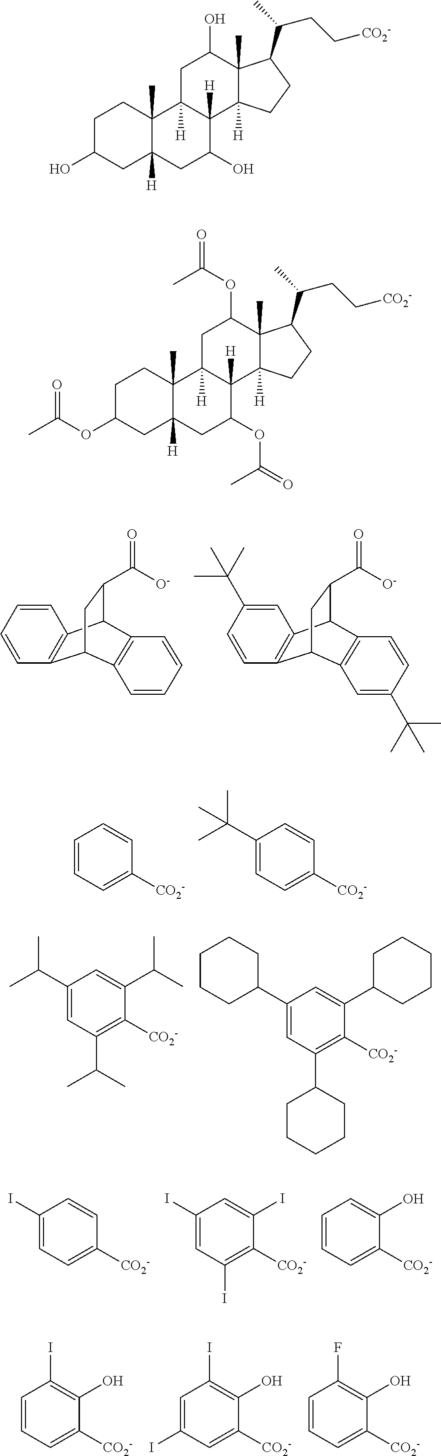

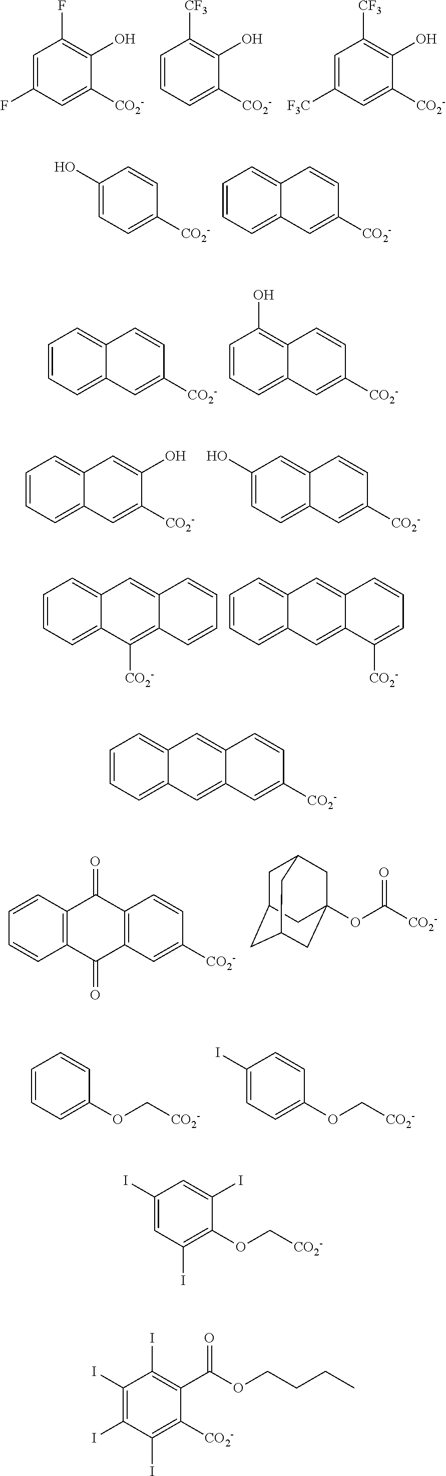

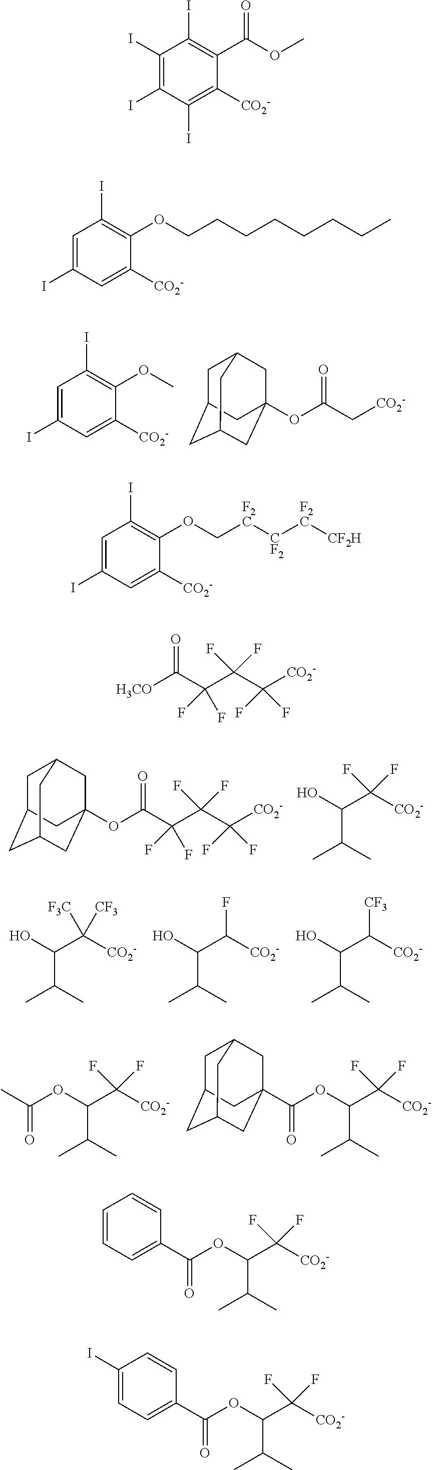

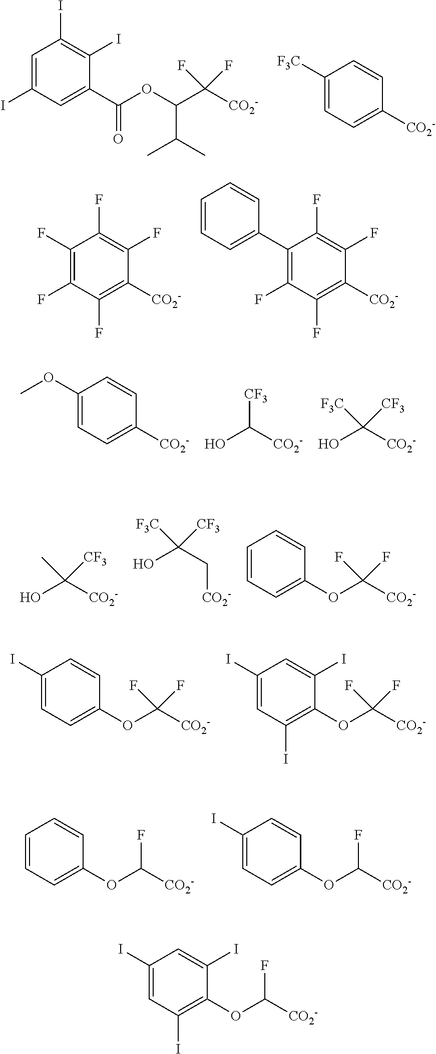

In formulae (5) and (6), Mq⁺ is an onium cation. Preferred examples of the onium cation include sulfonium, iodonium and ammonium cations. Examples of the sulfonium cation are as described in JP-A 2024-003744, paragraphs [0102]-[0125] and JP-A 2023-169812, paragraphs [0070]-[0085], and include cations in the sulfonium salts of formula (1), but are not limited thereto. Examples of the iodonium cation are as described in JP-A 2024-000259, paragraph [0181], but not limited thereto.

Preferably, the ammonium cation has the formula (am-1).

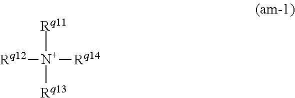

In formula (am-1), R^{q11} to R^{q14} are each independently a C₁-C₄₀ hydrocarbyl group which may contain a heteroatom. Any two of R^{q11} to R^{q14} are optionally bonded to each other to form a ring together with a nitrogen atom to which these groups are bonded. Examples of the hydrocarbyl group are as exemplified above for the hydrocarbyl groups R¹ to R⁴ in formula (1).

Examples of the ammonium cation having formula (am-1) are shown below, but not limited thereto.

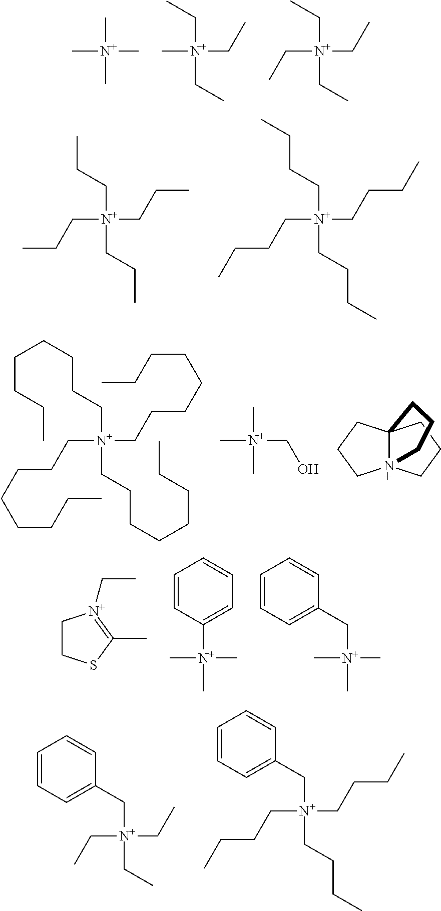

Examples of the onium salt having formula (5) or (6) include arbitrary combinations of anions with cations, both as exemplified above. These onium salts may be readily prepared by ion exchange reaction using any well-known organic chemistry technique. For the ion exchange reaction, reference may be made to JP-A 2007-145797, for example.

The onium salt of formula (5) or (6) functions as a quencher in the chemically amplified resist composition. This is because the counter anion of the onium salt is a conjugated base of a weak acid. As used herein, the weak acid indicates an acidity insufficient to deprotect an acid labile group from an acid labile group-containing unit for the base polymer. The onium salt having formula (5) or (6) functions as a quencher when used in combination with an onium salt type PAG having a conjugated base of a strong acid (typically sulfonic acid) as the counter anion. In a system using a mixture of an onium salt capable of generating a strong acid (typically sulfonic acid) and an onium salt capable of generating a weak acid (typically carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into an acid having a low catalysis, incurring apparent deactivation of the acid for enabling to control acid diffusion.

Also useful as the other quencher (E) are onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755.

If a photoacid generator capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the onium salt having formula (5) or (6) is used as the quencher (E), the amount of the onium salt used is preferably 0.1 to 20 parts by weight, more preferably 0.1 to 10 parts by weight per 80 parts by weight of the base polymer (B). As long as the amount of onium salt type quencher (E) is in the range, a satisfactory resolution is available without a substantial lowering of sensitivity. The onium salt having formula (5) or (6) may be used alone or in admixture.

Nitrogen-containing compounds may also be used as the other quencher (E). Suitable nitrogen-containing compounds include primary, secondary and tertiary amine compounds, specifically amine compounds having a hydroxy group, ether bond, ester bond, lactone ring, cyano group or sulfonate ester bond, as described in JP-A 2008-111103, paragraphs [0146]-[0164](U.S. Pat. No. 7,537,880), and primary or secondary amine compounds protected with a carbamate group, as described in JP 3790649.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the nitrogen-containing compound is used as the other quencher (E), the amount of the nitrogen-containing compound used is preferably 0.001 to 12 parts by weight, more preferably 0.01 to 8 parts by weight per 80 parts by weight of the base polymer (B). The nitrogen-containing compound may be used alone or in admixture.

### (F) Surfactant

The chemically amplified resist composition of the invention may further comprise (F) a surfactant. Preferred are a surfactant which is insoluble or substantially insoluble in water and soluble in alkaline developer, and a surfactant which is insoluble or substantially insoluble in water and alkaline developer. For the surfactant, reference should be made to those compounds described in JP-A 2010-215608 and JP-A 2011-16746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® 5-381, KH-20 and KH-30 (AGC Seimi Chemical Co., Ltd.). Partially fluorinated oxetane ring-opened polymers having the formula (surf-1) are also useful.

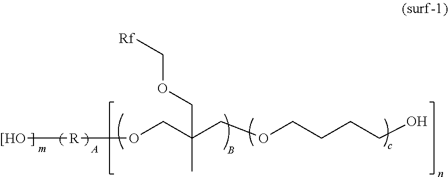

It is provided herein that R, Rf, A, B, C, m, and n are applied to only formula (surf-1), independent of the above descriptions. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

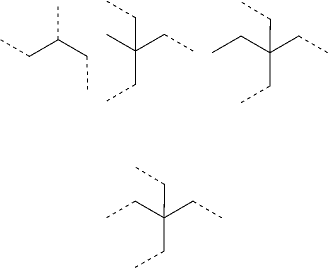

Herein the broken line denotes a valence bond. These formulae are partial structures derived from glycerol, trimethylol ethane, trimethylol propane, and pentaerythritol, respectively.

Of these, 1,4-butylene and 2,2-dimethyl-1,3-propylene are preferred.

Rf is trifluoromethyl group or pentafluoroethyl group, preferably trifluoromethyl group. The subscript m is an integer of 0 to 3, n is an integer of 1 to 4, and the sum of m and n, which represents the valence of R, is an integer of 2 to 4. A is 1. B is an integer of 2 to 25, preferably an integer of 4 to 20. C is an integer of 0 to 10, preferably 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (6A) to (6E).

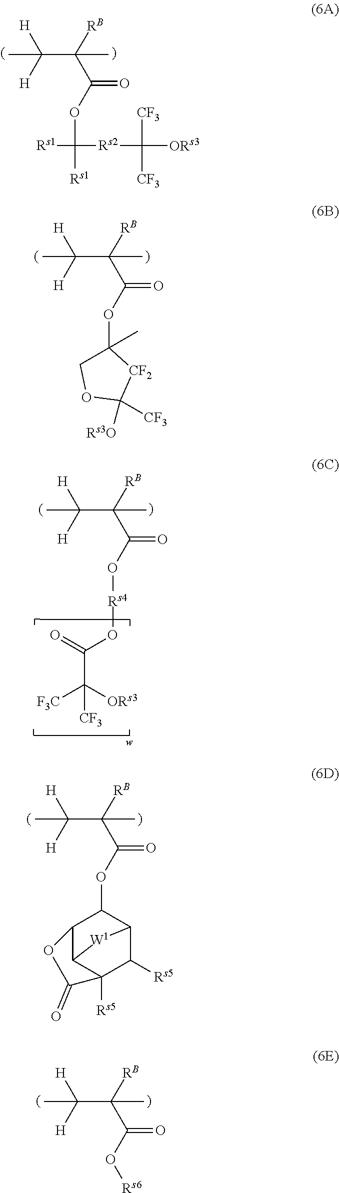

In formulae (6A) to (6E), R^{B} is hydrogen, fluorine, methyl or trifluoromethyl. W¹ is —CH₂—, —CH₂CH₂— or —O—, or two separate —H. R^{s1} is each independently hydrogen or a C₁-C₁₀ hydrocarbyl group. R^{s2} is a single bond or C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, ether bond or carbonyl moiety may intervene in a carbon-carbon bond. R^{s4} is a C₁-C₂₀ (u+1)-valent hydrocarbon or fluorinated hydrocarbon group. The subscript n2 is 1, 2 or 3. R^{s5} is each independently hydrogen or a group: —C(═O)—O—R^{sa}. R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The C₁-C₁₀ hydrocarbyl group R^{s1} may be straight, branched or cyclic and is preferably saturated. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ hydrocarbyl groups are preferred.

The hydrocarbylene group R^{s2} may be straight, branched or cyclic and is preferably saturated. Examples thereof include methylene, ethylene, propylene, butylene and pentylene groups.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group R^{s1} as well as undecyl, dodecyl, tridecyl, tetradecyl, and pentadecyl. Examples of the fluorinated hydrocarbyl group R^{s3} or R^{s6} include the foregoing hydrocarbyl groups in which some or all carbon-bonded hydrogen is substituted by fluorine. In these groups, an ether bond or carbonyl moiety may intervene in a carbon-carbon bond as mentioned above.

Examples of the acid labile group R^{s3} include the groups of formulae (AL-3) to (AL-5), trialkylsilyl groups in which each alkyl group is a C₁-C₆ alkyl group, and C₄-C₂₀ oxoalkyl groups.

The (u+1)-valent hydrocarbon or fluorinated hydrocarbon group R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group R^{sa} is preferably saturated while it may be straight, branched or cyclic. Examples thereof include the foregoing hydrocarbyl groups in which some or all hydrogen is substituted by fluorine. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units of formulae (6A) to (6E) are shown below, but not limited thereto. Herein R^{B} is as defined above.

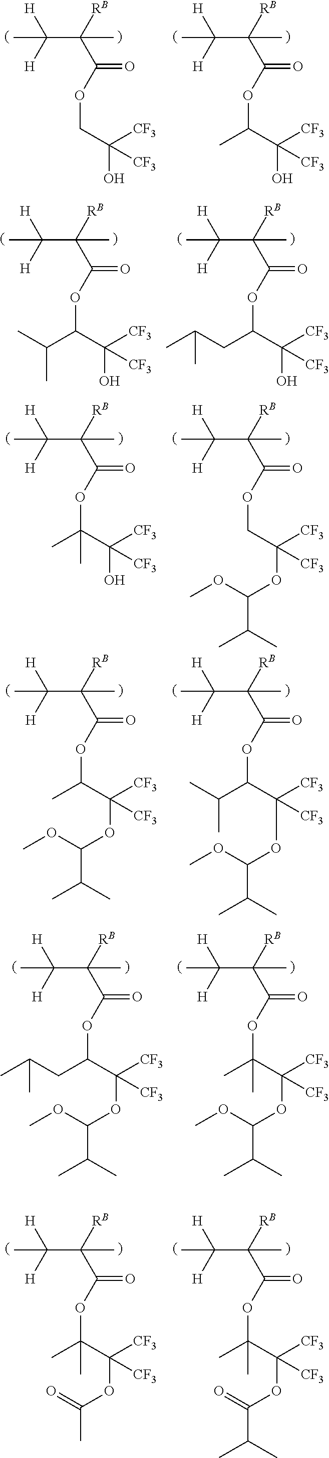

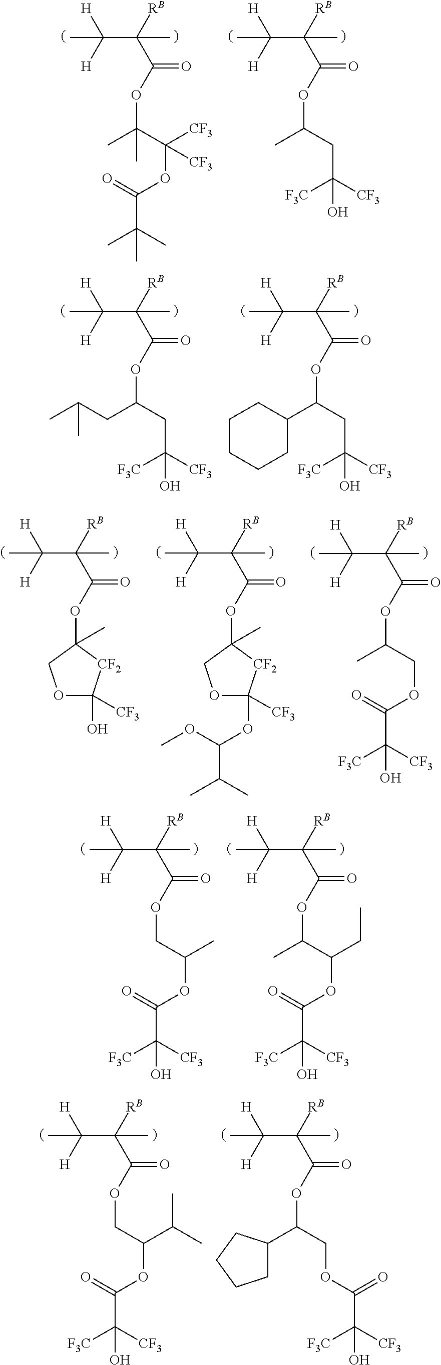

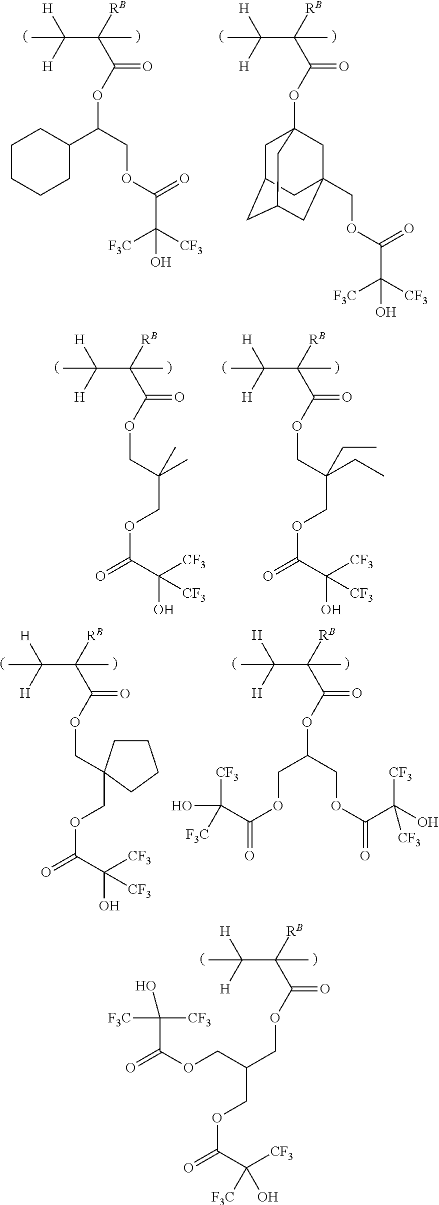

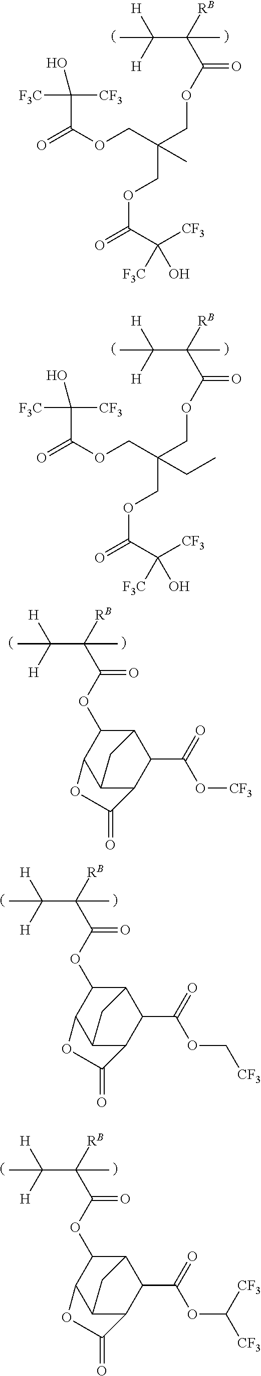

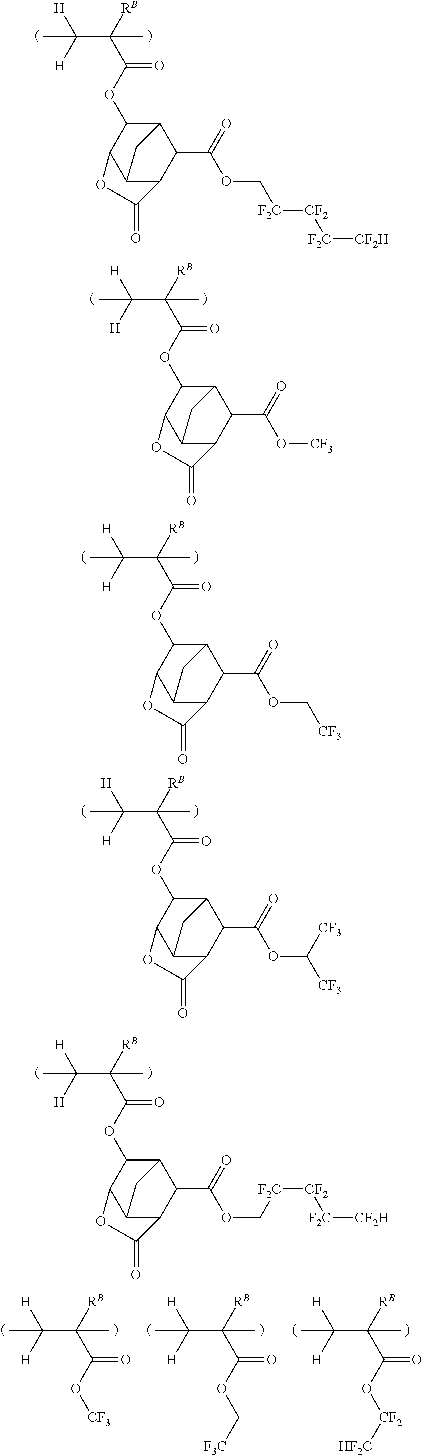

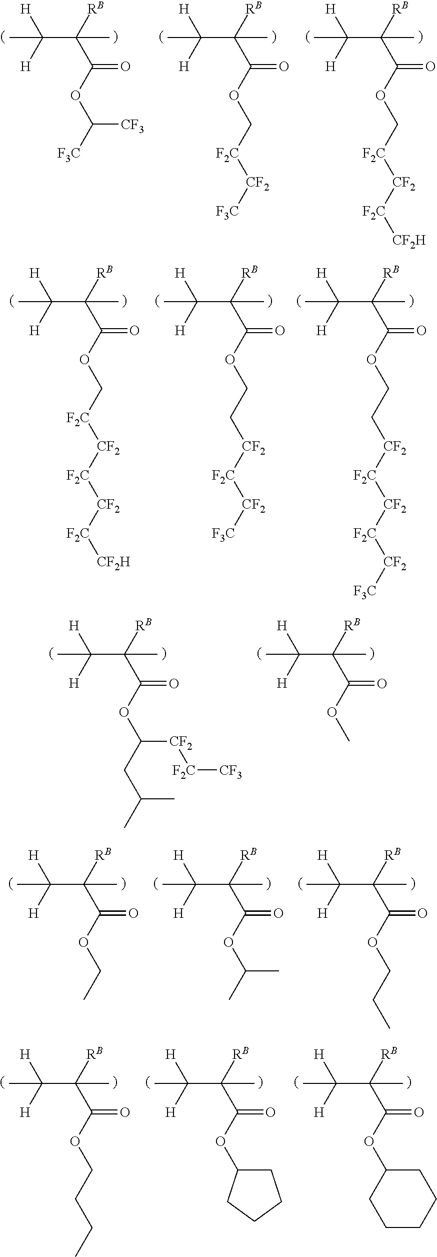

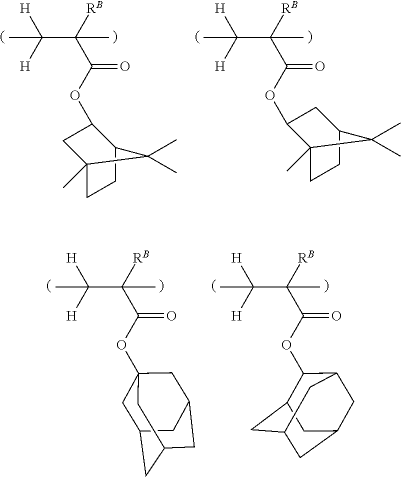

The polymeric surfactant may further contain repeat units other than the repeat units having formulae (6A) to (6E). Typical other repeat units are, for example, those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

Mw of the polymeric surfactant is preferably 1,000 to 500,000, more preferably 3,000 to 100,000. Mw/Mn is preferably 1.0 to 2.0, more preferably 1.0 to 1.6.

The polymeric surfactant may be synthesized, for example, by dissolving an unsaturated bond-containing monomer or monomers, from which repeat units having formulae (6A) to (6E) and optional other repeat units are derived, in an organic solvent, adding a radical initiator, and heating for polymerization. Examples of the suitable organic solvent used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or the polymerization may be followed by protection or partial protection.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the chemically amplified resist composition contains the surfactant (F), the amount of the surfactant (E) used is 0.1 to 50 parts by weight, more preferably 0.5 to 10 parts by weight per 80 parts by weight of the base polymer (B). At least 0.1 part of the surfactant is effective in improving the receding contact angle with water of the resist film at its surface. Up to 50 parts of the surfactant is effective in forming a resist film having a low rate of dissolution in a developer and capable of maintaining the height of a small-size pattern formed therein. The surfactant (F) may be used alone or in admixture.

### (G) Other Components

The chemically amplified resist composition of the invention may further comprise (G) another component, for example, a compound which is decomposed with an acid to generate another acid (i.e., acid amplifier compound), an organic acid derivative, a fluorinated alcohol, and a compound having a Mw of up to 3,000 which changes its solubility in developer under the action of an acid (i.e., dissolution inhibitor). The acid amplifier compound is described in JP-A 2009-269953 and JP-A 2010-215608. The acid amplifier compound is preferably used in an amount of 0 to 5 parts by weight, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative, fluorinated alcohol and dissolution inhibitor, reference should be made to JP-A 2009-269953 and JP-A 2010-215608.

### Process

A further embodiment of the invention is a process of forming a pattern from the chemically amplified resist composition defined above by lithography. The preferred process includes the steps of applying the resist composition onto a substrate to form a resist film thereon, exposing the resist film to high-energy radiation, and developing the exposed resist film in a developer. Any desired steps may be added to the process if necessary.

The substrate used herein may be a substrate for integrated circuitry fabrication, e.g., Si, SiO₂, SiN, SiON, TiN, WSi, BPSG, SOG, organic antireflective film, etc. or a substrate for mask circuitry fabrication, e.g., Cr, CrO, CrON, MoSi₂, SiO₂, etc.

The chemically amplified resist composition is applied by a suitable coating technique such as spin coating. The coating is prebaked on a hot plate preferably at a temperature of 60 to 150° C. for 1 to 10 minutes, more preferably at 80 to 140° C. for 1 to 5 minutes. The resulting resist film preferably has a thickness of preferably 0.05 to 2 μm.

Then the resist film is exposed to a pattern of high-energy radiation, typically KrF or ArF excimer laser, EUV or EB. On use of KrF excimer laser, ArF excimer laser or EUV, the resist film is exposed through a mask having a desired pattern, preferably in a dose of 1 to 200 mJ/cm², more preferably 10 to 100 mJ/cm². On use of EB, a pattern may be written directly or through a mask having the desired pattern, preferably in a dose of 1 to 300 μC/cm², more preferably 10 to 200 μC/cm².

The exposure may be performed by conventional lithography whereas the immersion lithography of holding a liquid having a refractive index of at least 1.0 between the resist film and the projection lens may be employed if desired. In the case of immersion lithography, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

The exposure may be followed by PEB. The resist film may be baked (PEB), for example, on a hotplate preferably at 60 to 150° C. for 1 to 5 minutes, more preferably at 80 to 140° C. for 1 to 3 minutes.

The resist film is developed in a developer in the form of an aqueous alkaline solution for preferably 0.1 to 3 minutes, more preferably 0.5 to 2 minutes by conventional techniques such as dip, puddle and spray techniques. A preferable developer is a 0.1 to 5 wt %, more preferably 2 to 3 wt % aqueous solution of tetramethylammonium hydroxide (TMAH) or another alkali. In this way, the exposed regions are dissolved, and the desired pattern is formed on the substrate.

After the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the inventive pattern forming process, a negative tone development method may also be used. That is, an organic solvent may be used instead of the aqueous alkaline solution as the developer for dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. The organic solvents may be used alone or in admixture.

### EXAMPLES

Synthesis Examples, Examples, and Comparative Examples of the invention are given below by way of illustration and not by way of limitation. The apparatuses used are as follows.
MALDI TOF-MS: S3000 manufactured by JEOL Ltd.

### [1] Synthesis of Onium Salts

### Example 1-1

### Synthesis of Onium Salt SQ-1

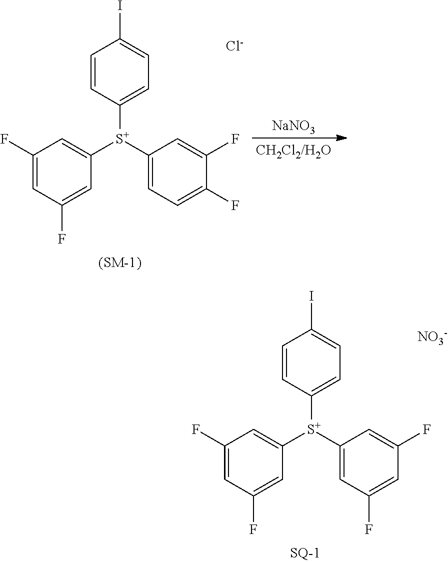

### (1) Synthesis of SQ-1

Under nitrogen atmosphere, 7.5 g of Reactant SM-1, 1.7 g of sodium nitrate, 40 g of methylene chloride, and 10 g of water were added, stirred for 15 minutes. Thereafter, the organic layer was taken out, washed with water, and then concentrated under reduced pressure. To the concentrate, 50 g of methyl isobutyl ketone was added, and water is removed by azeotropic distillation. Diisopropyl ether was then added to induce crystallization, obtaining the target SQ-1 as oily matter (amount 5.7 g, yield 73%).

### MALDI TOF-MS:

POSITIVE M⁺461 (corresponding to C₁₈H₁₀F₄S⁺)
NEGATIVE M⁻62 (corresponding to NO₃⁻)

### Examples 1-2 to 1-9

### Synthesis of Onium Salts SQ-2 to SQ-10

Onium Salt SQ-2 to SQ-10 of the following formulae were synthesized using the corresponding reactants and well-known organic chemistry reaction.

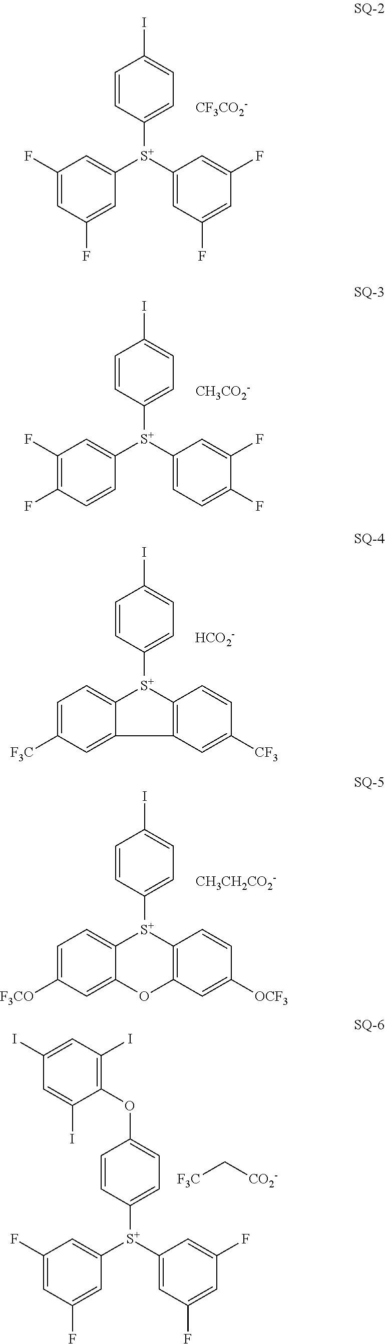

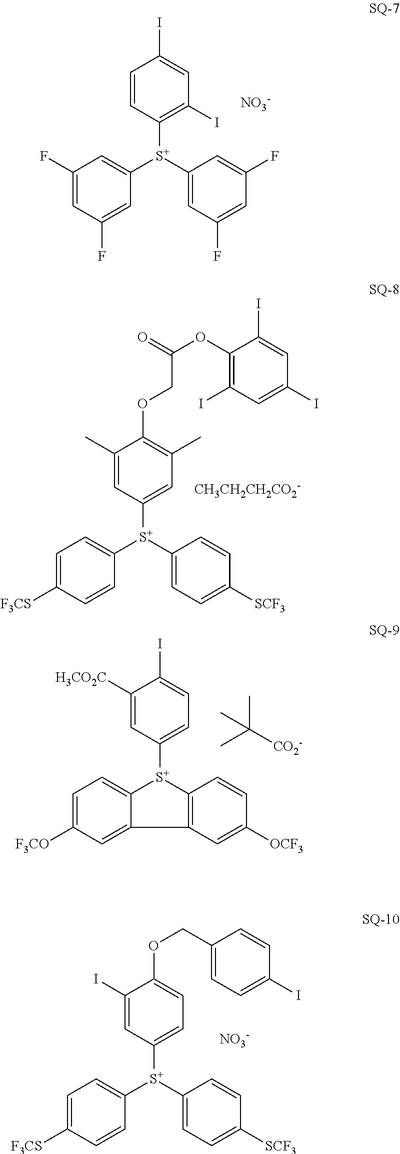

### [2] Synthesis of Base Polymer

### Synthesis Example

### Synthesis of Base Polymers (P-1 to P-5)

Base polymers P-1 to P-5 were synthesized by combining monomers, performing copolymerization reaction in MEK solvent, pouring the reaction solution to hexane for precipitation, washing the solid precipitate with hexane, isolation and drying. The base polymer was analyzed for composition by ¹H-NMR spectroscopy and for Mw and Mw/Mn by GPC versus polystyrene standards using DMF solvent.

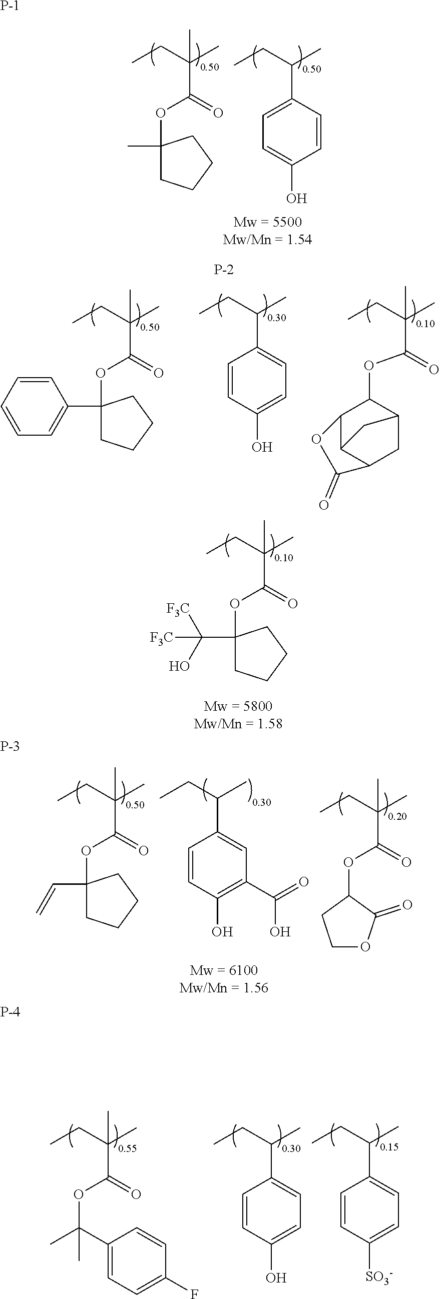

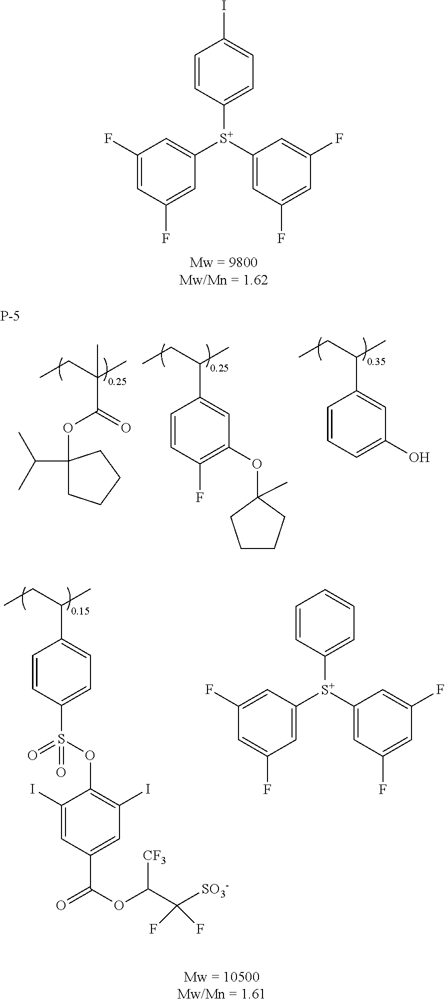

### [3] Preparation of Chemically Amplified Resist Composition

### Examples 2-1 to 2-45 and Comparative Examples 1-1 to 1-25

Chemically amplified resist compositions (R-1 to R-45 and CR-1 to CR-25) in solution form were prepared by dissolving a quencher (SQ-1 to SQ-10) or comparative quencher (SQ-A to SQ-D, AQ-A), photoacid generator (PAG-X and PAG-Y), and base polymer (P-1 to P-5) in a solvent containing 0.01 wt % of surfactant A (OMNOVA Inc.) in accordance with the formulation shown in Tables 1 to 3, and filtering through a Teflon® filter with a pore size of 0.2 μm.

TABLE 1

| | | | | Base | Photoacid | | |
|---|---|---|---|---|---|---|---|
| | | Resist | Quencher | polymer | generator | Solvent 1 | Solvent 2 |
| | | composition | (pbw) | (pbw) | (pbw) | (pbw) | (pbw) |
| Example | 2-1 | R-1 | SQ-1 (8.0) | P-1 (80) | PAG-1 (24) | PGMEA (2200) | DAA (900) |
| | 2-2 | R-2 | SQ-2 (7.8) | P-1 (80) | PAG-1 (24) | PGMEA (2200) | DAA (900) |
| | 2-3 | R-3 | SQ-3 (8.2) | P-1 (80) | PAG-1 (24) | PGMEA (2200) | DAA (900) |
| | 2-4 | R-4 | SQ-4 (8.0) | P-1 (80) | PAG-1 (24) | PGMEA (2200) | DAA (900) |
| | 2-5 | R-5 | SQ-5 (7.8) | P-1 (80) | PAG-1 (24) | PGMEA (2200) | DAA (900) |
| | 2-6 | R-6 | SQ-6 (8.0) | P-1 (80) | PAG-1 (24) | PGMEA (2200) | DAA (900) |
| | 2-7 | R-7 | SQ-7 (7.6) | P-1 (80) | PAG-1 (24) | PGMEA (2200) | DAA (900) |
| | 2-8 | R-8 | SQ-8 (7.8) | P-1 (80) | PAG-1 (26) | PGMEA (2200) | DAA (900) |
| | 2-9 | R-9 | SQ-9 (8.0) | P-1 (80) | PAG-1 (24) | PGMEA (2200) | DAA (900) |
| | 2-10 | R-10 | SQ-10 (8.0) | P-1 (80) | PAG-1 (24) | PGMEA (2200) | DAA (900) |
| | 2-11 | R-11 | SQ-1 (8.0) | P-1 (80) | PAG-2 (26) | PGMEA (2200) | DAA (900) |
| | 2-12 | R-12 | SQ-2 (8.2) | P-1 (80) | PAG-2 (24) | PGMEA (2200) | DAA (900) |
| | 2-13 | R-13 | SQ-3 (7.8) | P-1 (80) | PAG-2 (26) | PGMEA (2200) | DAA (900) |
| | 2-14 | R-14 | SQ-1 (8.0) | P-2 (80) | PAG-1 (24) | PGMEA (2200) | DAA (900) |
| | 2-15 | R-15 | SQ-2 (7.6) | P-2 (80) | PAG-2 (24) | PGMEA (2200) | DAA (900) |
| | 2-16 | R-16 | SQ-3 (8.0) | P-2 (80) | PAG-1 (25) | PGMEA (2200) | DAA (900) |
| | 2-17 | R-17 | SQ-4 (8.0) | P-2 (80) | PAG-2 (24) | PGMEA (2200) | DAA (900) |
| | 2-18 | R-18 | SQ-7 (7.6) | P-2 (80) | PAG-1 (24) | PGMEA (2200) | DAA (900) |
| | 2-19 | R-19 | SQ-1 (8.0) | P-3 (80) | PAG-1 (26) | PGMEA (2200) | DAA (900) |
| | 2-20 | R-20 | SQ-2 (8.2) | P-3 (80) | PAG-1 (24) | PGMEA (2200) | DAA (900) |
| | 2-21 | R-21 | SQ-3 (7.8) | P-3 (80) | PAG-1 (24) | PGMEA (2200) | DAA (900) |
| | 2-22 | R-22 | SQ-5 (8.0) | P-3 (80) | PAG-2 (24) | PGMEA (2200) | DAA (900) |
| | 2-23 | R-23 | SQ-2 (4.0) | P-3 (80) | PAG-1 (24) | PGMEA (2200) | DAA (900) |
| | | | SQ-7 (3.6) | | | | |

TABLE 2

| | | | | Base | Photoacid | | |
|---|---|---|---|---|---|---|---|
| | | Resist | Quencher | polymer | generator | Solvent 1 | Solvent 2 |
| | | composition | (pbw) | (pbw) | (pbw) | (pbw) | (pbw) |
| Example | 2-24 | R-24 | SQ-1 (8.0) | P-4 (80) | — | PGMEA (2200) | DAA (900) |
| | 2-25 | R-25 | SQ-2 (8.0) | P-4 (80) | — | PGMEA (2200) | DAA (900) |
| | 2-26 | R-26 | SQ-3 (7.8) | P-4 (80) | — | PGMEA (2200) | DAA (900) |
| | 2-27 | R-27 | SQ-4 (8.0) | P-4 (80) | — | PGMEA (2200) | DAA (900) |
| | 2-28 | R-28 | SQ-5 (8.2) | P-4 (80) | — | PGMEA (2200) | DAA (900) |
| | 2-29 | R-29 | SQ-6 (8.0) | P-4 (80) | — | PGMEA (2200) | DAA (900) |
| | 2-30 | R-30 | SQ-7 (8.0) | P-4 (80) | — | PGMEA (2200) | DAA (900) |
| | 2-31 | R-31 | SQ-8 (7.8) | P-4 (80) | — | PGMEA (2200) | DAA (900) |
| | 2-32 | R-32 | SQ-9 (8.0) | P-4 (80) | — | PGMEA (2200) | DAA (900) |
| | 2-33 | R-33 | SQ-10 (8.0) | P-4 (80) | — | PGMEA (2200) | DAA (900) |
| | 2-34 | R-34 | SQ-1 (7.8) | P-4 (80) | PAG-1 (10) | PGMEA (2200) | DAA (900) |
| | 2-35 | R-35 | SQ-2 (8.0) | P-4 (80) | PAG-2 (10) | PGMEA (2200) | DAA (900) |
| | 2-36 | R-36 | SQ-1 (7.6) | P-5 (80) | — | PGMEA (2200) | DAA (900) |
| | 2-37 | R-37 | SQ-2 (8.0) | P-5 (80) | — | PGMEA (2200) | DAA (900) |
| | 2-38 | R-38 | SQ-3 (8.0) | P-5 (80) | — | PGMEA (2200) | DAA (900) |
| | 2-39 | R-39 | SQ-4 (7.8) | P-5 (80) | — | PGMEA (2200) | DAA (900) |
| | 2-40 | R-40 | SQ-5 (7.6) | P-5 (80) | — | PGMEA (2200) | DAA (900) |
| | 2-41 | R-41 | SQ-1 (4.0) | P-5 (80) | — | PGMEA (2200) | DAA (900) |
| | | | SQ-3 (3.6) | | | | |
| | 2-42 | R-42 | SQ-7 (8.0) | P-5 (80) | — | PGMEA (2200) | DAA (900) |
| | 2-43 | R-43 | SQ-8 (7.8) | P-5 (80) | — | PGMEA (2200) | DAA (900) |
| | 2-44 | R-44 | SQ-1 (8.0) | P-5 (80) | PAG-1 (10) | PGMEA (2200) | DAA (900) |
| | 2-45 | R-45 | SQ-1 (8.0) | P-5 (80) | PAG-2 (10) | PGMEA (2200) | DAA (900) |

TABLE 3

| | | | | Base | Photoacid | | |
|---|---|---|---|---|---|---|---|
| | | Resist | Quencher | polymer | generator | Solvent 1 | Solvent 2 |
| | | composition | (pbw) | (pbw) | (pbw) | (pbw) | (pbw) |
| Comparative | 1-1 | CR-1 | SQ-A (8.0) | P-1 (80) | PAG-1 (24) | PGMEA (2200) | DAA (900) |
| Example | 1-2 | CR-2 | SQ-B (7.8) | P-1 (80) | PAG-1 (24) | PGMEA (2200) | DAA (900) |
| | 1-3 | CR-3 | SQ-C (8.0) | P-1 (80) | PAG-1 (24) | PGMEA (2200) | DAA (900) |
| | 1-4 | CR-4 | SQ-D (8.2) | P-1 (80) | PAG-1 (24) | PGMEA (2200) | DAA (900) |
| | 1-5 | CR-5 | AQ-A (8.0) | P-1 (80) | PAG-1 (24) | PGMEA (2200) | DAA (900) |
| | 1-6 | CR-6 | SQ-A (8.0) | P-1 (80) | PAG-2 (26) | PGMEA (2200) | DAA (900) |
| | 1-7 | CR-7 | SQ-B (8.2) | P-1 (80) | PAG-2 (24) | PGMEA (2200) | DAA (900) |
| | 1-8 | CR-8 | SQ-A (7.8) | P-2 (80) | PAG-1 (24) | PGMEA (2200) | DAA (900) |
| | 1-9 | CR-9 | SQ-C (8.0) | P-2 (80) | PAG-1 (24) | PGMEA (2200) | DAA (900) |
| | 1-10 | CR-10 | AQ-A (8.0) | P-2 (80) | PAG-2 (26) | PGMEA (2200) | DAA (900) |
| | 1-11 | CR-11 | SQ-A (8.0) | P-3 (80) | PAG-1 (24) | PGMEA (2200) | DAA (900) |
| | 1-12 | CR-12 | SQ-B (4.0) | P-3 (80) | PAG-1 (24) | PGMEA (2200) | DAA (900) |
| | | | AQ-A (3.6) | | | | |
| | 1-13 | CR-13 | SQ-C (8.2) | P-3 (80) | PAG-2 (26) | PGMEA (2200) | DAA (900) |
| | 1-14 | CR-14 | SQ-A (8.0) | P-4 (80) | — | PGMEA (2200) | DAA (900) |
| | 1-15 | CR-15 | SQ-B (8.0) | P-4 (80) | — | PGMEA (2200) | DAA (900) |
| | 1-16 | CR-16 | SQ-C (7.8) | P-4 (80) | — | PGMEA (2200) | DAA (900) |
| | 1-17 | CR-17 | SQ-D (8.0) | P-4 (80) | — | PGMEA (2200) | DAA (900) |
| | 1-18 | CR-18 | AQ-A (8.0) | P-4 (80) | — | PGMEA (2200) | DAA (900) |
| | 1-19 | CR-19 | SQ-A (7.8) | P-4 (80) | PAG-1 (10) | PGMEA (2200) | DAA (900) |
| | 1-20 | CR-20 | SQ-C (8.0) | P-4 (80) | PAG-2 (10) | PGMEA (2200) | DAA (900) |
| | 1-21 | CR-21 | SQ-A (7.8) | P-5 (80) | — | PGMEA (2200) | DAA (900) |
| | 1-22 | CR-22 | SQ-B (8.0) | P-5 (80) | — | PGMEA (2200) | DAA (900) |
| | 1-23 | CR-23 | SQ-C (8.2) | P-5 (80) | — | PGMEA (2200) | DAA (900) |
| | 1-24 | CR-24 | SQ-D (8.0) | P-5 (80) | — | PGMEA (2200) | DAA (900) |
| | 1-25 | CR-25 | SQ-A (4.0) | P-5 (80) | PAG-1 (10) | PGMEA (2200) | DAA (900) |
| | | | SQ-3 (3.6) | | | | |

The solvents, photoacid generators PAG-X and PAG-Y, comparative quenchers SQ-A to SQ-D, AQ-A and surfactant A in Tables 1 to 3 are identified below.

### Solvent:

PGMEA (propylene glycol monomethyl ether acetate)
DAA (diacetone alcohol)
   Acid generator: PAG-X, PAG-Y

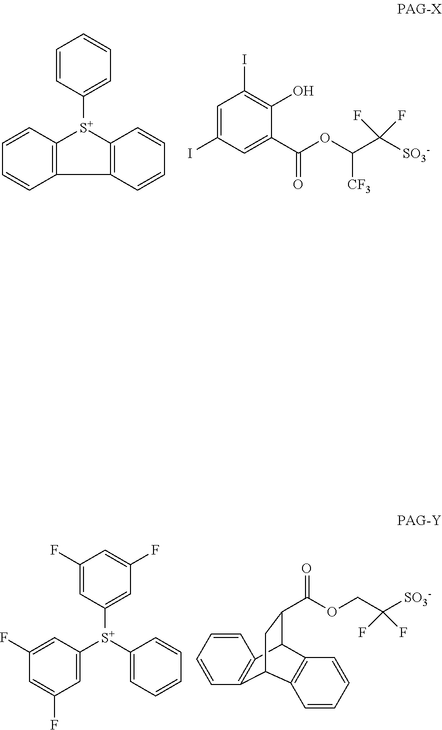

Comparative quencher: SQ-A to SQ-D, AQ-A

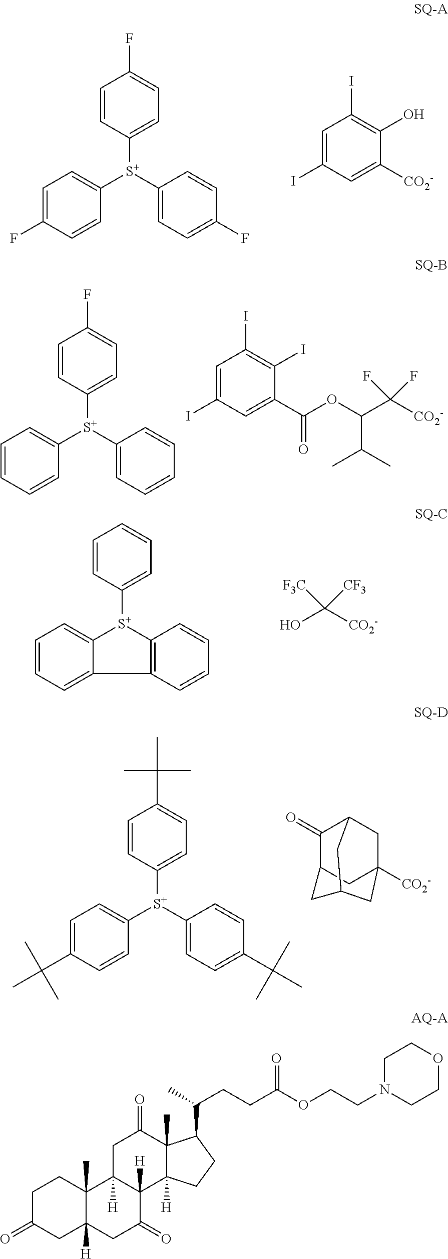

### Surfactant A:

3-methyl-3-(2,2,2-trifluoroethoxymethyl)oxetane/tetrahydrofuran/
2,2-dimethyl-1,3-propanediol copolymer (manufactured by OMNOVA Inc.)

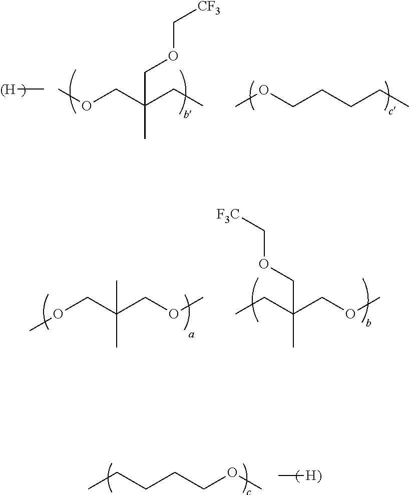

a:(b+b′):(c+c′)=1:4 to 7:0.01 to 1 (molar ratio)
Mw=1,500

### [4] EUV Lithography Test (1)

### Examples 3-1 to 3-45 and Comparative Examples 2-1 to 2-25

Each of the chemically amplified resist compositions (R-1 to R-45, CR-1 to CR-25) shown in Tables 1 to 3 was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3400 manufactured by ASML (NA 0.33, a 0.9/0.6, dipole illumination), the resist film was exposed to EUV through a mask bearing a line-and-space (LS) pattern having a width of 18 nm (on-wafer size) and a pitch of 36 nm while changing the dose at a pitch of 1 mJ/cm² and the focus at a pitch of 0.020 μm. The resist film was baked (PEB) at the temperature shown in Tables 4 and 5 for 60 seconds. This was followed by puddle development in a 2.38 wt % TMAH aqueous solution for seconds, rinsing with a surfactant-containing rinse fluid, and spin drying. A positive LS pattern was obtained.

The LS pattern as developed was observed under CD-SEM (CG6300, Hitachi High-Technologies Corp.) whereupon sensitivity, EL, LWR, depth of focus (DOF), and collapse limit were evaluated by the following methods. The results are shown in Tables 4 to 6.

### [Evaluation of Sensitivity]

The optimum dose Eop (mJ/cm²) which provided an LS pattern with a line width of 18 nm and a pitch of 36 nm was determined as an index of sensitivity. A smaller value indicates a higher sensitivity.

### [Evaluation of EL]

The exposure dose which provided an LS pattern with a space width of 18 nm±10% (i.e., 16.2 to 19.8 nm) was determined. EL (%) is calculated from the exposure doses according to the following equation: A greater value indicates better performance.

$$EL ( \% ) = ( \vert {E}_{1} - {E}_{2} \vert / Eop ) \times 100$$

wherein E₁ is an optimum exposure dose which provides an LS pattern with a line width of 16.2 nm and a pitch of 36 nm,
E₂ is an optimum exposure dose which provides an LS pattern with a line width of 19.8 nm and a pitch of 36 nm, and
Eop is an optimum exposure dose which provides an LS pattern with a line width of 18 nm and a pitch of 36 nm.

### [Evaluation of LWR]

For the LS pattern formed by exposure at the optimum dose Eop, the line width was measured at 10 longitudinally spaced apart points, from which a 3-fold value (3σ) of standard deviation (σ) was determined and reported as LWR. A smaller value of 3a indicates a pattern having small roughness and uniform line width.

### [Evaluation of DOF]

As an index of DOF, a range of focus which provided an LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 4

| | | | PEB | Optimal | | | | Collapse |
|---|---|---|---|---|---|---|---|---|
| | | Resist | temp. | exposure dose | EL | LWR | DOF | limit |
| | | composition | (° C.) | (mJ/cm²) | (%) | (nm) | (nm) | (nm) |
| Example | 3-1 | R-1 | 100 | 33 | 18 | 2.4 | 120 | 10.6 |
| | 3-2 | R-2 | 105 | 34 | 19 | 2.4 | 110 | 10.7 |
| | 3-3 | R-3 | 100 | 33 | 17 | 2.5 | 120 | 10.9 |
| | 3-4 | R-4 | 105 | 34 | 18 | 2.4 | 110 | 11.1 |
| | 3-5 | R-5 | 105 | 33 | 17 | 2.4 | 100 | 11.2 |
| | 3-6 | R-6 | 100 | 35 | 17 | 2.5 | 120 | 11.0 |
| | 3-7 | R-7 | 100 | 34 | 18 | 2.6 | 110 | 10.8 |
| | 3-8 | R-8 | 95 | 35 | 19 | 2.7 | 100 | 11.3 |
| | 3-9 | R-9 | 100 | 34 | 17 | 2.6 | 110 | 11.5 |
| | 3-10 | R-10 | 105 | 34 | 19 | 2.4 | 120 | 11.3 |
| | 3-11 | R-11 | 105 | 34 | 19 | 2.6 | 120 | 11.2 |
| | 3-12 | R-12 | 100 | 35 | 17 | 2.5 | 110 | 11.2 |
| | 3-13 | R-13 | 105 | 33 | 18 | 2.6 | 120 | 11.5 |
| | 3-14 | R-14 | 100 | 35 | 17 | 2.6 | 100 | 11.5 |
| | 3-15 | R-15 | 105 | 34 | 17 | 2.5 | 110 | 11.2 |
| | 3-16 | R-16 | 95 | 34 | 18 | 2.4 | 120 | 11.3 |
| | 3-17 | R-17 | 100 | 34 | 17 | 2.4 | 110 | 10.8 |
| | 3-18 | R-18 | 95 | 35 | 18 | 2.5 | 110 | 11.1 |
| | 3-19 | R-19 | 95 | 33 | 19 | 24 | 120 | 11.4 |
| | 3-20 | R-20 | 100 | 34 | 17 | 2.6 | 110 | 10.9 |
| | 3-21 | R-21 | 100 | 33 | 19 | 2.5 | 110 | 11.0 |
| | 3-22 | R-22 | 100 | 33 | 17 | 2.6 | 120 | 11.2 |
| | 3-23 | R-23 | 95 | 35 | 17 | 2.4 | 110 | 11.3 |

TABLE 5

| | | | PEB | Optimal | | | | Collapse |
|---|---|---|---|---|---|---|---|---|
| | | Resist | temp. | exposure dose | EL | LWR | DOF | limit |
| | | composition | (° C.) | (mJ/cm²) | (%) | (nm) | (nm) | (nm) |
| Example | 3-24 | R-24 | 105 | 33 | 18 | 2.6 | 120 | 11.3 |
| | 3-25 | R-25 | 100 | 35 | 17 | 2.6 | 100 | 11.5 |
| | 3-26 | R-26 | 105 | 35 | 17 | 2.5 | 110 | 11.2 |
| | 3-27 | R-27 | 95 | 34 | 18 | 2.4 | 120 | 11.6 |
| | 3-28 | R-28 | 100 | 34 | 17 | 2.4 | 110 | 11.0 |
| | 3-29 | R-29 | 95 | 35 | 18 | 2.5 | 110 | 11.4 |
| | 3-30 | R-30 | 105 | 33 | 19 | 24 | 120 | 11.5 |
| | 3-31 | R-31 | 100 | 34 | 17 | 2.6 | 110 | 11.4 |
| | 3-32 | R-32 | 105 | 33 | 19 | 2.5 | 110 | 11.2 |
| | 3-33 | R-33 | 100 | 33 | 19 | 2.4 | 110 | 11.2 |
| | 3-34 | R-34 | 100 | 34 | 19 | 2.6 | 120 | 11.2 |
| | 3-35 | R-35 | 95 | 35 | 17 | 2.4 | 110 | 11.3 |
| | 3-36 | R-36 | 105 | 4 | 18 | 2.6 | 120 | 11.3 |
| | 3-37 | R-37 | 100 | 35 | 17 | 2.6 | 100 | 11.0 |
| | 3-38 | R-38 | 105 | 35 | 17 | 2.5 | 110 | 11.2 |
| | 3-39 | R-39 | 100 | 34 | 18 | 2.4 | 120 | 11.0 |
| | 3-40 | R-40 | 100 | 34 | 17 | 2.4 | 110 | 11.8 |
| | 3-41 | R-41 | 105 | 35 | 18 | 2.5 | 110 | 11.4 |
| | 3-42 | R-42 | 95 | 33 | 18 | 2.4 | 120 | 11.5 |
| | 3-43 | R-43 | 105 | 34 | 17 | 2.6 | 110 | 11.4 |
| | 3-44 | R-44 | 100 | 33 | 19 | 2.5 | 110 | 11.2 |
| | 3-45 | R-45 | 105 | 34 | 17 | 2.5 | 110 | 11.0 |

TABLE 6

| | | | PEB | Optimal | | | | Collapse |
|---|---|---|---|---|---|---|---|---|
| | | Resist | temp. | exposure dose | EL | LWR | DOF | limit |
| | | composition | (° C.) | (mJ/cm²) | (%) | (nm) | (nm) | (nm) |
| Comparative | 2-1 | CR-1 | 100 | 36 | 15 | 2.9 | 90 | 12.9 |
| Example | 2-2 | CR-2 | 105 | 37 | 14 | 2.8 | 80 | 12.8 |
| | 2-3 | CR-3 | 100 | 36 | 14 | 2.9 | 80 | 13.1 |
| | 2-4 | CR-4 | 105 | 35 | 14 | 2.7 | 90 | 12.9 |
| | 2-5 | CR-5 | 105 | 41 | 14 | 3.7 | 70 | 13.8 |
| | 2-6 | CR-6 | 100 | 38 | 15 | 2.9 | 90 | 12.6 |
| | 2-7 | CR-7 | 100 | 40 | 13 | 2.8 | 90 | 12.8 |
| | 2-8 | CR-8 | 100 | 38 | 14 | 3.1 | 80 | 12.5 |
| | 2-9 | CR-9 | 95 | 39 | 14 | 3.2 | 90 | 12.7 |
| | 2-10 | CR-10 | 100 | 39 | 14 | 3.0 | 80 | 12.3 |
| | 2-11 | CR-11 | 105 | 40 | 14 | 2.8 | 70 | 12.8 |
| | 2-12 | CR-12 | 95 | 38 | 14 | 2.9 | 80 | 12.6 |
| | 2-13 | CR-13 | 100 | 39 | 15 | 3.1 | 70 | 13.1 |
| | 2-14 | CR-14 | 105 | 40 | 14 | 3.4 | 80 | 13.5 |
| | 2-15 | CR-15 | 100 | 41 | 15 | 2.8 | 80 | 13.2 |
| | 2-16 | CR-16 | 95 | 42 | 14 | 2.9 | 80 | 12.8 |
| | 2-17 | CR-17 | 105 | 39 | 14 | 3.1 | 70 | 12.6 |
| | 2-18 | CR-18 | 100 | 38 | 15 | 3.2 | 80 | 12.9 |
| | 2-19 | CR-19 | 105 | 37 | 14 | 3.1 | 80 | 13.0 |
| | 2-20 | CR-20 | 95 | 38 | 14 | 2.8 | 90 | 13.1 |
| | 2-21 | CR-21 | 100 | 37 | 15 | 2.9 | 90 | 13.2 |
| | 2-22 | CR-22 | 105 | 37 | 14 | 3 | 80 | 13.5 |
| | 2-23 | CR-23 | 100 | 39 | 14 | 3.2 | 80 | 12.8 |
| | 2-24 | CR-24 | 105 | 40 | 15 | 3.1 | 70 | 13.2 |
| | 2-25 | CR-25 | 100 | 39 | 14 | 2.8 | 80 | 13.4 |

It is demonstrated in Tables 4 to 6 that chemically amplified resist compositions comprising a quencher comprising an onium salt within the scope of the invention exhibit a high sensitivity and improved EL, LWR and DOF. The resist composition is also confirmed to have a low collapse resistance value, and resistance to pattern collapse in fine pattern formation. This demonstrates that chemically amplified resist compositions are suitable as materials for EUV lithography.

Japanese Patent Application No. 2024-106248 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 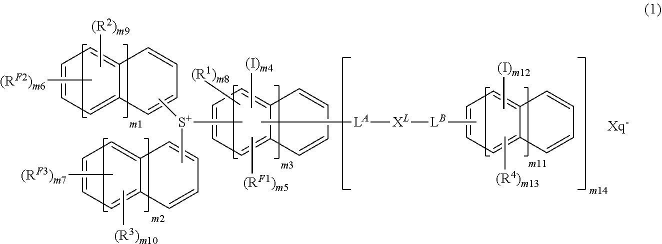
wherein m1 is 0 or 1, m2 is 0 or 1, m3 is 0 or 1, m4 is 0, 1, 2, 3 or 4, m5 is 0, 1, 2, 3 or 4, m6 is 0, 1, 2, 3, 4, 5 or 6, m7 is 0, 1, 2, 3, 4, 5 or 6, m7 is 0, 1, 2, 3, 4, 5 or 6, m8 is 0, 1 or 2, m9 is 0, 1 or 2, m10 is 0, 1 or 2, m11 is 0 or 1, m12 is 0, 1, 2, 3 or 4, m13 is 0, 1 or 2, m14 is 0, 1 or 2, meeting 0≤m6+m9≤4 in case of m1=0 and 0≤m6+m9≤6 in case of m1=1, 0≤m7+m10≤4 in case of m2=0 and 0≤m7+m10≤6 in case of m2=1, 1≤m4+m5+m8+m14≤4 in case of m3=0 and 1≤m4+m5+m8+m14≤6 in case of m3=1, 0≤m12+m13≤4 in case of m11=0 and 0≤m12+m13≤6 in case of m11=1, and m4+m12≥1,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group, a plurality of RF¹ may be identical or different when m5 is 2 or more, a plurality of RF² may be identical or different when m6 is 2 or more, a plurality of RF³ may be identical or different when m7 is 2 or more,
R¹ to R⁴ each are halogen exclusive of iodine and fluorine, nitro group, cyano group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that when m8 is 2, two R¹ may be identical or different and two R¹ may bond together to form a ring with the carbon atoms to which they are attached, when m9 is 2, two R² may be identical or different and two R² may bond together to form a ring with the carbon atoms to which they are attached, when m10 is 2, two R³ may be identical or different and two R³ may bond together to form a ring with the carbon atoms to which they are attached, when m13 is 2, two R⁴ may be identical or different and two R⁴ may bond together to form a ring with the carbon atoms to which they are attached,
the aromatic rings directly bonded to S⁺ in the sulfonium cation may bond together to form a ring with S⁺,
L^{A} and L^{B} are each independently a single bond, ether bond, ester bond, amide bond, sulfonate ester bond, sulfonamide bond, carbonate bond or carbamate bond,
X^{L} is a single or a C₁-C₄₀ hydrocarbylene group which may contain a heteroatom, and
Xq⁻ is an anion, with the proviso that an acid (XqH) whose conjugated base is Xq⁻ has a boiling point of lower than 165° C. and a molecular weight of 150 or less.

2. The onium salt of claim 1, wherein the conjugate acid XqH of the anion Xq⁻ is formic acid, acetic acid, propionic acid, butyric acid, trifluoroacetic acid, 3,3,3-trifluoropropionic acid, pivalic acid or nitric acid.

3. The onium salt of claim 1 which has the formula (1A): 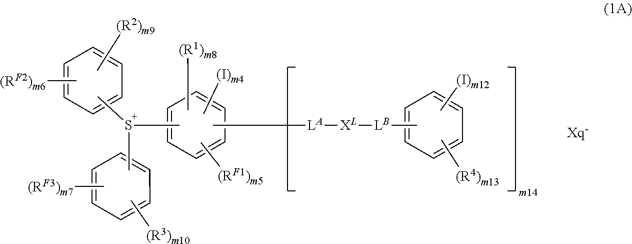
wherein m4 to m10, m12 to m14, R^{F1} to R^{F3}, R¹ to R⁴, L^{A}, L^{B}, X^{L} and Xq⁻ are as defined above.

4. The onium salt of claim 3 which has the formula (1B): 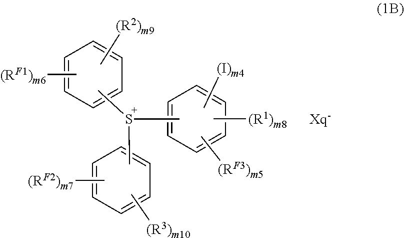
wherein m4 to m10, R^{F1} to R^{F3}, R¹ to R³ and Xq⁻ are as defined above.

5. A quencher in the form of the onium salt of claim 1.

6. A chemically amplified resist composition comprising the quencher of claim 5.

7. The chemically amplified resist composition of claim 6, further comprising a base polymer comprising repeat units of at least one type selected from repeat units having the formulae (a1) and (a2): 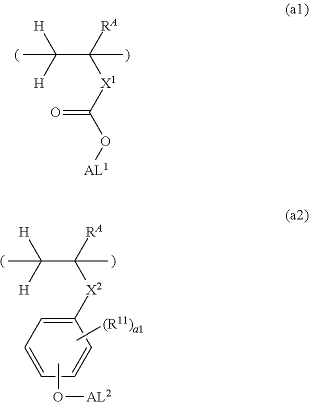
wherein R^{A} is each independently hydrogen, fluorine, methyl or trifluoromethyl,
X¹ is a single bond, phenylene group, naphthylene group, *—C(═O)—O—X¹¹— or *—C(═O)—NH—X¹¹—, the phenylene or naphthylene group may be substituted with a hydroxy moiety, nitro group, cyano group, optionally fluorinated C₁-C₁₀ saturated hydrocarbyl group, optionally fluorinated C₁-C₁₀ saturated hydrocarbyloxy group or halogen, X¹¹ is a C₁-C₁₀ saturated hydrocarbylene group, phenylene group or naphthylene group, the saturated hydrocarbylene group may contain a hydroxy moiety, ether bond, ester bond or lactone ring,
X² is a single bond, *—C(═O)—O— or *—C(═O)—NH—,
* designates a point of attachment to the carbon in the backbone,
R¹¹ is halogen, cyano, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, a C₂-C₂₀ hydrocarbyloxycarbonyl group which may contain a heteroatom, a plurality of R¹¹ may be identical or different when a1 is 2, 3 or 4,
AL¹ and AL² are each independently an acid labile group, and
a1 is 0, 1, 2, 3 or 4.

8. The chemically amplified resist composition of claim 7, wherein the base polymer further comprises repeat units having the formula (a3): 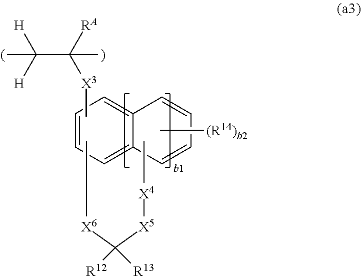
wherein b1 is 0 or 1, and b2 is 0, 1, 2 or 3 in case of b1=0 and 0, 1, 2, 3, 4 or 5 in case of b1=1,
R^{A} is hydrogen, fluorine, methyl or trifluoromethyl,
X³ is a single bond, *—C(═O)—O—, or *—C(═O)—NH—, and * designates a point of attachment to the carbon atom in the backbone,
X⁴ is a single bond, C₁-C₄ aliphatic hydrocarbylene group, carbonyl group, sulfonyl group, or a group obtained by combining the foregoing,
X⁵ and X⁶ are each independently oxygen or sulfur, X⁴ and X⁶ are bonded to vicinal carbon atoms on the aromatic ring,
R¹² and R¹³ are each independently hydrogen, or a C₁-C₂₀ hydrocarbyl group which may contain a heteroatom, R¹² and R¹³ may bond together to form a ring with the carbon atoms to which they are attached,
R¹⁴ is halogen, hydroxy, cyano, nitro, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may be identical or different and a plurality of R¹⁴ may bond together to form a ring with the carbon atoms on the aromatic ring to which they are attached when b2 is 2 or more.

9. The chemically amplified resist composition of claim 7, wherein the base polymer further comprises repeat units of at least one type selected from repeat units having the formulae (b1) and (b2): 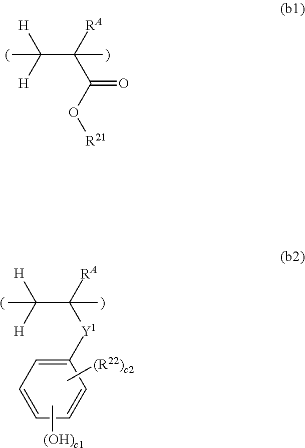
wherein R^{A} is hydrogen, fluorine, methyl or trifluoromethyl,
Y¹ is a single bond or *—C(═O)—O—, * designates a point of attachment to the carbon atom in the backbone,
R²¹ is hydrogen or a C₁-C₂₀ group containing at least one structure selected from among hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—),
R²² is halogen, hydroxy, carboxy, nitro, cyano, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3 or 4,
c1 is 1, 2, 3 or 4, c2 is 0, 1, 2, 3 or 4, and c1+c2 is from 1 to 5.

10. The chemically amplified resist composition of claim 7, wherein the base polymer further comprises repeat units of at least one type selected from repeat units having the formulae (c1), (c2), (c3), (c4) and (c5): 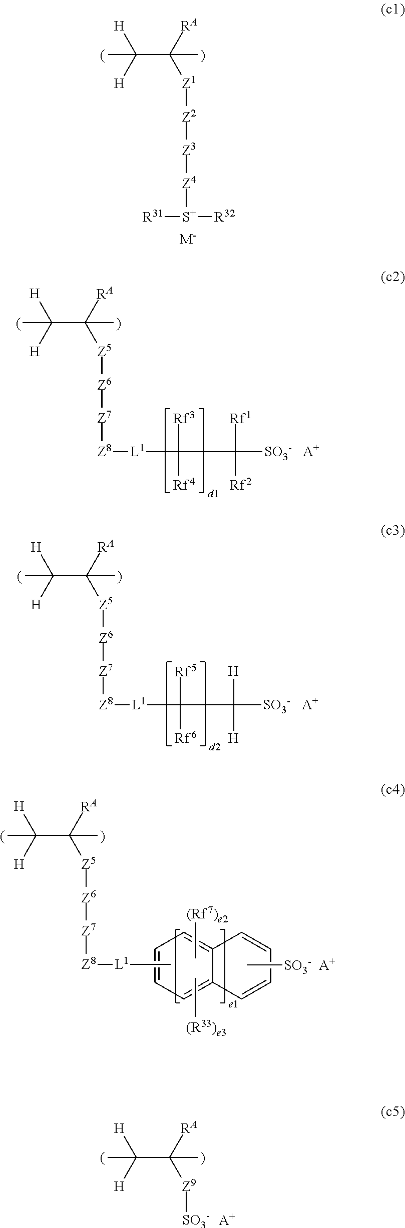
wherein d1 and d2 are each independently 0, 1, 2 or 3, e1 is 0 or 1, e2 is 0, 1, 2, 3 or 4, e3 is 0, 1, 2, 3 or 4, meeting 0≤e2+e3≤4 in case of e1=0 and 0≤e2+e3≤6 in case of e1=1,
R^{A} is each independently hydrogen, fluorine, methyl or trifluoromethyl,
Z¹ is a single bond or optionally substituted phenylene group,
Z² is a single bond, **—C(═O)—O—Z²¹—, **—C(═O)—NH—Z²¹— or **—O—Z²¹—, Z²¹— is a C₁-C₆ aliphatic hydrocarbylene group, phenylene group or a divalent group obtained by combining the foregoing, which may contain halogen, a carbonyl moiety, ester bond, ether bond or hydroxy moiety,
Z³ is a single bond, ether bond, ester bond, amide bond, sulfonate ester bond, sulfonamide bond, carbonate bond or carbamate bond,
Z⁴ is a C₁-C₆ aliphatic hydrocarbylene group, phenylene group or a divalent group obtained by combining the foregoing, which may contain halogen, a carbonyl moiety, ester bond, ether bond or hydroxy moiety,
Z⁵ is each independently a single bond, optionally substituted phenylene group, naphthylene group, or *—C(═O)—O—Z⁵¹, Z⁵¹ is a C₁-C₁₀ aliphatic hydrocarbylene group which may contain halogen, hydroxy moiety, ether bond, ester bond or lactone ring, or phenylene or naphthylene group,
Z⁶ is a single bond, ether bond, ester bond, amide bond, sulfonate ester bond, sulfonamide bond, carbonate bond or carbamate bond,
Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, methylene, ethylene, phenylene, fluorinated phenylene, trifluoromethyl-substituted phenylene, *—C(═O)—O—Z⁹¹—, *—C(═O)—N(H)—Z⁹¹—, or *—O—Z⁹¹—, Z⁹¹ is a C₁-C₆ aliphatic hydrocarbylene group, phenylene group, fluorinated phenylene group or trifluoromethyl-substituted phenylene group, which may contain a carbonyl moiety, ester bond, ether bond or hydroxy moiety,
* designates a point of attachment to the carbon atom in the backbone, ** designates a point of attachment to Z¹, *** designates a point of attachment to Z⁶, **** designates a point of attachment to Z⁷,
L¹ is a single bond, ether bond, ester bond, carbonyl moiety, sulfonate ester bond, sulfonamide bond, carbonate bond or carbamate bond,
Rf¹ and Rf² are each independently fluorine or a C₁-C₆ fluorinated saturated hydrocarbyl group,
Rf³ and Rf⁴ are each independently hydrogen, fluorine, or a C₁-C₆ fluorinated saturated hydrocarbyl group,
Rf⁵ and Rf⁶ are each independently hydrogen, fluorine, or a C₁-C₆ fluorinated saturated hydrocarbyl group, excluding that all Rf⁵ and Rf⁶ are hydrogen at the same time,
Rf⁷ is fluorine, a C₁-C₆ fluorinated alkyl group, a C₁-C₆ fluorinated alkoxy group, a C₁-C₆ fluorinated alkylthio group,
R³¹ and R³² are each independently a C₁-C₂₀ hydrocarbyl group which may contain a heteroatom, R³¹ and R³² may bond together to form a ring with the sulfur atom to which they are attached,
R³³ is halogen exclusive of fluorine, or a C₁-C₂₀ hydrocarbyl group which may contain a heteroatom, a plurality of R³³ may be identical or different and a plurality of R³³ may bond together to form a ring with the carbon atoms to which they are attached when e3 is 2, 3 or 4,
M⁻ is a non-nucleophilic counter ion, and
A⁺ is an onium cation.

11. The chemically amplified resist composition of claim 6, further comprising an organic solvent.

12. The chemically amplified resist composition of claim 6, further comprising a photoacid generator.

13. The chemically amplified resist composition of claim 6, further comprising a quencher other than the quencher.

14. The chemically amplified resist composition of claim 6, further comprising a surfactant.

15. A pattern forming process comprising the steps of applying the chemically amplified resist composition of claim 6 onto a substrate to form a resist film thereon, exposing the resist film to high-energy radiation, and developing the exposed resist film in a developer.

16. The pattern forming process of claim 15, wherein the high-energy radiation is KrF excimer laser, ArF excimer laser, an electron beam, or an extreme ultraviolet ray having a wavelength 3 to 15 nm.
